SEQUENCE LISTING (7 sequences)

SEQ ID NO: 1 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 2 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 3 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 4 - protein, 148 aa, Mus musculus
SSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 5 - protein, 142 aa, Homo sapiens
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRD

SEQ ID NO: 6 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 7 - protein, 133 aa, Artificial Sequence
  Synthetic construct
QVSHRYPRIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

(19) United States
(12) Patent Application Publication
(10) Pub. No.: US 2011/0028688 A1
(43) Pub. Date: Feb. 3, 2011

(54) CRYSTAL STRUCTURE OF OX40L AND OX40L COMPLEXED WITH OX40 RECEPTOR

(75) Inventors: Sarah Hymowitz, San Francisco, CA (US); Deanne Compaan McGuinn, San Francisco, CA (US)

Correspondence Address: MORRISON & FOERSTER LLP, 755 PAGE MILL RD, PALO ALTO, CA, 94304-1018, US

(73) Assignee: GENENTECH, INC., South San Francisco, CA (US)

(21) Appl. No.: 12/305,638

(22) PCT Filed: Jun. 19, 2007
(86) PCT No.: PCT/US2007/071596

§ 371 Date: Dec. 10, 2009

Related U.S. Application Data
(60) Provisional application No. 60/805,433, filed on Jun. 21, 2006.

(51) Int. Cl. C07K 14/435; G06F 17/50; G06F 17/30

(52) U.S. Cl. 530/350; 703/1; 707/769; 707/E17.014

(57) ABSTRACT

The present disclosure provides crystals of and structural coordinates of mOX40L, hOX40L, hOX40 receptor and complexes thereof. The crystals and crystal structures are useful, for example, in the design and synthesis of modulators of mOX40L, hOX40L, and/or hOX40 receptor.

### CROSS REFERENCE TO RELATED APPLICATION

This application is a International Application which claims priority to U.S. Ser. No. 60/805,433, filed Jun. 21, 2006, which application is hereby incorporated by reference.

### BACKGROUND OF THE INVENTION

Many members of the Tumor Necrosis Factor superfamily (TNFSF) and their receptors modulate immune system activity either by triggering apoptosis or, conversely, by acting as growth or survival factors (Locksley et al., Cell, 104:487-501 (2001)). OX40 (also known as TNFRSF4, ACT35, TXGP1L or CD134) along with TNFRSF family members 4-1BB, CD27, CD30, and CD40 are co-stimulatory molecules acting at different stages in T and B cell activation to modulate and control the immune response (Watts, Annu Rev Immunol. 23:23-68 (2005)). The OX40-OX40L pair function relatively late in T cell activation. OX40L (also known as TNFSF4, gp34, TXGP1) is expressed on the surface of antigen presenting cells (APCs) approximately 1 to 3 days after initial antigen encounter and interacts with OX40 expressed on activated T cells (Croft, Cytokine Growth Factor Rev, 14:265-273 (2003)). This interaction results in recruitment of TRAF2 and signals for T cell survival in a survivin dependent manner (Song et al., Immunity, 22:621-631 (2005)). OX40 activity is implicated in the generation of activated TH1 and TH2 cells as well as the maintenance of memory T cell populations (Croft, cited supra; Weinberg et al., J Leukoc Biol, 75:962-972 (2004)).

The cytokine, TSLP, which has a robust association with TH2 pathologies in human disease, has recently been shown to trigger OX40L expression on dendritic cells (Ito et al., J Exp Med, 202:1213-1223 (2005). Additionally, OX40 and OX40L knock-out mice are deficient in TH2 responses suggesting that TH2 polarization is the pathway most influenced by OX40 signaling (Jember et al., J Exp Med, 193:387-392 (2001); Hoshino et al., Eur J Immunol, 33:861-869 (2003)). Consistent with this hypothesis, treatment with anti-OX40L monoclonal antibodies which block interaction with OX40 results in reduced cytokine production, antigen specific IgE levels and lung inflammation in murine models of allergic asthma (Hoshino et al., cited supra). Blocking of OX40-OX40L interaction in vivo has been shown to reduce severity of disease in acute graft vs. host disease, inflammatory bowel disease, and collagen induced arthritis (Weinberg et al., Trends in Immunology, 23:102 (2002)).

Despite their varied biological roles, TNFSF ligands are generally homotrimeric proteins composed of three jelly roll protomers. Each protomer is formed by two (3-sheets which contain strands A′AHCF and B′BGDE. Most TNFSF ligands are type II transmembrane proteins and several, such as FasL, TNF, BAFF, and EDA, have processing sites allowing them to be released from the cell surface to act as soluble factors (Bodmer et al., Trends Biochem Sci, 27:19-26 (2002)). The TNFSF ligands can roughly be divided into three groups based on sequence and structural features: the conventional, the EF-disulfide containing, and the divergent.

The “conventional” TNFSF members include TNF, LT [symbol] , LT [symbol] , Apo2L/TRAIL, TL1A, LIGHT, FasL, RANKL and CD40L. Crystal structures are available for TNF, LTα, Apo21/TRAIL, RANKL and CD40L. These structures and models indicate that these ligands all have relatively long loops connecting the CD, DF and DE strands giving the trimers a characteristic pyramidal shape. The “conventional” ligands all have, in the DE loop, a conserved hydrophobic residue (generally a tyrosine) which has been shown to be energetically important for receptor binding in several of the family members.

The second TNFSF ligand group, the “EF-disulfide” group, includes APRIL, BAFF, TWEAK and EDA. Crystal structures are available for APRIL, BAFF and EDA (Hymowitz et al., Biochemistry, 39:633-640 (2000); Karpusas et al., J Mol Biol, 315:1145-1154 (2002); Wallweber et al., J Mol Biol, 343:283-290 (2004)). These ligands all possess a disulfide connecting the E and F strands and are characterized by shorter CD and EF loops resulting in a more globular shape. Receptor binding by this TNFSF group also differs from the conventional ligand group as they lack the conserved hydrophobic residues in the DE loop. In addition, three (APRIL, BAFF and TWEAK) of the these four ligands have been shown to bind very small atypical TNFRSF members (BR3, TACI, BCMA, and Fn14) (Bodmer et al., cited supra).

The third “divergent” ligand group includes CD27L, CD30L, GITRL, 4-1BBL, and OX40L. These ligands all have sequences that are very divergent from each other and from either the “conventional” or “EF-disulfide” groups. No structural or mutagenesis data is available to validate if or how these ligands assemble into trimers or interact with receptors. OX40L is an example of this group. It is one of the most divergent members of the TNFSF with only ˜10-15% sequence identity to other family members and is also very compact with only ˜132 residues in the entire extra cellular region of human OX40L. In addition to the lack of sequence homology within the TNF-domain, OX40L also has an usually short linker between the extracellular TNF homology domain and the transmembrane helix with no discernable proteolytic site and thus is expected to exist only in a membrane bound state (Baum et al., Embo J, 13:3992-4001 (1994); Godfrey et al., J Exp Med, 180:757-762 (1994)).

In contrast to the globular ligands, TNFRSF receptors are elongated molecules composed of an extracellular domain of ˜40 residue pseudo repeats typically containing 6 cysteines forming 3 disulfide bonds. These modules are termed CRDs (cysteine rich domains) and can be further subdivided into smaller submodules based on the number of cysteines and topology of the cysteine connectivity (Naismith and Sprang, Trends Biochem Sci, 23:74-79 (1998)). A typical CRD is composed of A1 and B2 tandemly linked subdomains. The A1 subdomain contains a single disulfide (the 1-2 disulfide) while the B2 subdomain contains two disulfides which are linked in a 3-5, 4-6 topology. Other subdomain variants exist such as the A2 which contains two disulfides or the B1 which lacks one of the characteristic disulfides. Sequence analysis suggests that, unlike OX40L, OX40 is a relatively conventional member of the TNFRSF. The extracellular ligand binding domain of OX40 is composed of three full CRD and a partial, fourth C-terminal CRD (Godfrey, W. R. et al., cited supra). Both CRD1 and CRD2 have the prototypical A1-B2 linkage while CRD3 is an atypical CRD with the more unusual A1-B1 linkage.

As discussed previously, OX40 is implicated in the generation of TH1 and TH2 cells. The T-helper cell subsets (TH1 and TH2) define 2 pathways of immunity: cell-mediated immunity and humoral immunity. Release profiles of cytokines for TH1 and TH2 subtypes influence selection of effector mechanisms and cytotoxic cells (Mosmann et al., Adv. Immunol., 46:111-147 (1989); Mosmann et al., Immunol. Today, 17:138-146 (1996)). TH1 cells, a functional subset of CD4⁺ cells, are characterized by their ability to boost cell-mediated immunity and produce cytokines including Il-2, interferon-gamma, and lymphotoxin beta (Mosmann et al., 1989, 1996, supra). Il-2 and interferon-gamma secreted by TH1 cells activate macrophages and cytotoxic cells. TH2 cells are also CD4+ cells, but are distinct from TH1 cells. TH2 cells are characterized by their ability to boost humoral immunity, such as antibody production. TH2 cells produce cytokines, including Il-4, Il-5, and Il-10 (Mosmann et al., 1989, 1996, supra). Il-4, Il-5, and Il-10 secreted by TH2 cells increase production of eosinophils and mast cells, as well as enhance production of antibodies, including IgE, and decrease the function of cytotoxic cells (Powrie et al., Immunol. Today, 14:270 (1993)). Overproduction of cytokines produced by either or both of TH1 and TH2 cells impacts a host of medical disorders. For example, overproduction of TH1 cytokines contributes to pathogenesis of various autoimmune disorders, such as multiple sclerosis and rheumatoid arthritis. Overproduction of TH2 cytokines contributes to pathogenesis of allergic disorders.

There remains a need to develop new therapies useful to treat immune system diseases, autoimmune diseases, allergic disorders, and other diseases associated with TNF receptor signaling such as cancer.

### SUMMARY OF THE INVENTION

The present disclosure thus includes a crystalline form and a crystal structure of murine OX40L (mOX40L), a crystalline form and a crystal structure of human OX40L (hOX40L) complexed with a human OX40 receptor (hOX40), and a crystalline form and a crystal structure of mOX40L complexed with a hOX40 receptor. In other aspects, the disclosure provides methods of using the crystal structures and structural coordinates to identify homologous proteins and to design or identify agents that can modulate the function of the mOX40L, hOX40L, hOX40 receptor, and combinations thereof. The present disclosure also includes the three-dimensional configuration of points derived from the structure coordinates of at least a portion of a molecule or molecular complex, as well as structurally equivalent configurations, as described below. The three-dimensional configuration includes points derived from structure coordinates representing the locations of at least one or a plurality of the amino acids defining a binding site on mOX40L for a hOX40 receptor, a binding site on hOX40L for the hOX40 receptor, or a binding site on hOX40 receptor for either the mOX40L and/or hOX40L.

In some embodiments, the three-dimensional configuration includes points derived from structure coordinates representing the locations of the backbone atoms of a plurality of amino acids defining a binding site on mOX40L for the hOX40 receptor, a binding site on hOX40L for the hOX40 receptor, or a binding site on hOX40 receptor for either the mOX40L or hOX40L. Alternatively, the three-dimensional configuration includes points derived from structure coordinates representing the locations of the side chain and the backbone atoms (other than hydrogens) of a plurality of the amino acids defining a binding site on mOX40L for the hOX40 receptor, a binding site on hOX40L for the hOX40 receptor, or a binding site on hOX40 receptor for either the mOX40L or hOX40L.

In some embodiments, a crystal comprising a murine OX40L comprising SEQ ID NO:1, or a fragment of SEQ ID NO:1 comprising the sequence of amino acids 51 to 198 of SEQ ID NO:1 is provided. In some embodiments, a crystal of mOX40L diffracts Xrays to a resolution of 5 Å or better. In some embodiments, a crystal of mOX40L diffracts Xrays to a resolution of at least 1.45 to 2.5 Å or better. Also provided herein is a crystal of a fragment of mOX40L having a space group symmetry of P6₃ and comprising a unit cell having the dimensions of a=b and are about 74 Å, and c is about 48 Å. The unit cell dimensions can vary at least about plus or minus about 0.5 Å. The structural coordinates for mOX40L are provided in Table 8. In some embodiments, a composition comprises a crystal of mOX40L. Compositions and crystals of mOX40L may be a useful way to store, deliver or purify mOX40L.

In some embodiments, a cocrystal comprising a murine OX40L comprising SEQ ID NO:1, or a fragment of SEQ ID NO:1 comprising the sequence of amino acids 51 to 198 of SEQ ID NO:1 and hOX40 receptor comprising an amino acid sequence of SEQ ID NO:2 or a fragment of SEQ ID NO:2 comprising the sequence of amino acids 29 to 170 of SEQ ID NO:2 is provided. In some embodiments, a cocrystal of mOX40L-hOX40 receptor diffracts Xrays to a resolution of 5 Å or better. In some embodiments, a cocrystal of mOX40L-hOX40 receptor diffracts Xrays to a resolution of at least 1.45 to 2.5 Å or better. Also provided herein is a cocrystal of a fragment of mOX40L and a fragment of a hOX40 receptor having a space group symmetry of R32 and comprising a unit cell having the dimensions of a=b and are about 105 Å, and c is about 478 Å. The unit cell dimensions can vary at least about plus or minus about 0.5 Å. The structural coordinates for the cocrystal of mOX40L and hOX40L are provided in Table 9. In some embodiments, a composition comprises a cocrystal of mOX40L and hOX40 receptor. Compositions and crystals of mOX40L and hOX40 receptor may be a useful way to store, deliver or purify mOX40L and/or hOX40 receptor.

In some embodiments, a cocrystal comprising a human OX40L comprising SEQ ID NO:3, or a fragment of SEQ ID NO:3 comprising the sequence of amino acids 51 to 183 of SEQ ID NO:3 and hOX40 receptor comprising an amino acids sequence of SEQ ID NO:2 or a fragment of SEQ ID NO:2 comprising the sequence of amino acids 29 to 170 of SEQ ID NO:2 is provided. In some embodiments, a cocrystal of hOX40L-hOX40 receptor diffracts Xrays to a resolution of 5 Å or better. In some embodiments, a cocrystal of hOX40L-hOX40 receptor diffracts Xrays to a resolution of at least 1.45 to 2.5 Å or better. Also provided herein is a cocrystal of a fragment of hOX40L and a fragment of a hOX40 receptor having a space group symmetry of R32 and comprising a unit cell having the dimensions of a=b and are about 112 Å, and c is about 233 Å. The unit cell dimensions can vary at least about plus or minus about 0.5 Å. The structural coordinates for the cocrystal of hOX40L and hOX40L are provided in Table 10. In some embodiments, a composition comprises a cocrystal of hOX40L and hOX40 receptor. Compositions and crystals of hOX40L and hOX40 receptor may be a useful way to store, deliver or purify hOX40L and/or hOX40 receptor.

In another aspect, the disclosure includes mOX40L polypeptides and polynucleotides encoding the polypeptides. The disclosure includes a polynucleotide encoding a polypeptide and/or a polypeptide having at least 90% sequence identity to the polypeptide comprising the amino acid sequence of amino acids 51-198 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. Another embodiment, comprises a polynucleotide encoding a polypeptide and/or a polypeptide that comprises the amino acid sequence starting at any one of amino acids 51 to amino acid residue 66 and ending at any one of amino acids 180 to 191 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 64 to 190 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 99 to 191 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. The disclosure also includes a crystalline form of each of these polypeptides.

The present disclosure also includes an isolated polypeptide comprising, consisting essentially of, or consisting of a portion or fragment of the mOX40L. In some embodiments, the polypeptide comprises a binding site for hOX40 receptor. In other embodiments, the polypeptide comprises an extracellular domain of mOX40L. In other embodiments, the polypeptide comprises the trimer interface. In some embodiments, the trimer interface includes amino acid residues corresponding to amino acids 99 to 191 of SEQ. ID NO:1. An embodiment of a polypeptide comprises, consists essentially of, or consists of an amino acid residue starting from any one of amino acid residue 51 to amino acid residue 66 and ending at amino acid residue 180 to residue 191 or residues corresponding to those positions in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 51 to 198 in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 64 to 189 or 190 in the polypeptide comprising SEQ ID NO:1, excluding the amino acid sequence of SEQ ID NO:1. In some embodiments, the polypeptide has the ability to bind to hOX40 receptor. The disclosure also includes polynucleotides encoding such polypeptides and/or a hOX40 receptor ligand binding fragment thereof. The disclosure also includes a crystalline form of each of these polypeptides.

The disclosure also includes a polynucleotide encoding a polypeptide and/or a polypeptide having at least 90% sequence identity to the polypeptide comprising the amino acid sequence of amino acids 51 to 183 of SEQ ID NO:3, not including the polypeptide having the amino acid sequence of SEQ ID NO:3 or SEQ ID NO:1, and wherein the polypeptide binds a hOX40 receptor or a hOX40 receptor ligand binding fragment thereof. Another embodiment, comprises a polynucleotide encoding a polypeptide and/or a polypeptide that comprises the amino acid sequence starting at any one of amino acids 51 to amino acid residue 65 and ending at any one of amino acids 180 to 183 of SEQ ID NO:3, not including the polypeptide having the amino acid sequence of SEQ ID NO:3 or SEQ ID NO:1, and wherein the polypeptide binds a hOX40 receptor a hOX40 receptor ligand binding fragment thereof. In some embodiments, a polynucleotide encoding a polypeptide and/or a polypeptide has at least 90% sequence identity to the polypeptide comprising the amino acid sequence of amino acids 98 to 183 of SEQ ID NO:3, not including the polypeptide having the amino acid sequence of SEQ ID NO:3 or SEQ ID NO:1, and wherein the polypeptide binds a hOX40 receptor a hOX40 receptor ligand binding fragment thereof. The disclosure also includes a crystalline form of each of these polypeptides.

In another aspect, the disclosure includes hOX40L polypeptides and polynucleotides encoding the polypeptides. The present disclosure also includes an isolated polypeptide comprising, consisting essentially of, or consisting of a portion or fragment of the hOX40L. In some embodiments, the polypeptide comprises a binding site for hOX40 receptor. In other embodiments, the polypeptide comprises the trimer interface. In other embodiments, the polypeptide comprises an extracellular domain of hOX40L. An embodiment of a polypeptide comprises, consists essentially of, or consists of an amino acid residue starting from any one of amino acid residue 51 to amino acid residue 65 and ending at amino acid residue 180 to residue 183 or residues corresponding to those positions in the polypeptide comprising SEQ ID NO:3, excluding the amino acid sequence of SEQ ID NO:3. An embodiment of a polypeptide comprises, consists essentially of, or consists of an amino acid residues corresponding to amino acid residues 58 to 183 in the polypeptide comprising SEQ ID NO:3, excluding the amino acid sequence of SEQ ID NO:3. An embodiment of a polypeptide comprises, consists essentially of, or consists of an amino acid residues corresponding to amino acid residues 65 to 182 in the polypeptide comprising SEQ ID NO:3, excluding the amino acid sequence of SEQ ID NO:3. An embodiment of a polypeptide comprises, consists essentially of, or consists of an amino acid residues corresponding to amino acid residues 98 to 183 in the polypeptide comprising SEQ ID NO:3, excluding the amino acid sequence of SEQ ID NO:3. In some embodiments, the polypeptide portion has the ability to bind to hOX40 receptor and/or a hOX40 receptor ligand binding fragment thereof. The disclosure also includes polynucleotides encoding such polypeptides. The disclosure also includes a crystalline form of each of these polypeptides.

In another aspect, the disclosure includes hOX40 receptor polypeptides and polynucleotides encoding the polypeptides. The disclosure includes a polynucleotide encoding a polypeptide and/or a polypeptide having at least 90% sequence identity to the polypeptide comprising the amino acid sequence of amino acids 29 to 170 of SEQ ID NO:2, not including the polypeptide having the amino acid sequence of SEQ ID NO:2, and wherein the polypeptide binds hOX40L or a receptor binding fragment thereof. Another embodiment, comprises a polynucleotide encoding a polypeptide and/or a polypeptide that comprises the amino acid sequence starting at any one of amino acids 29 to amino acid residue 42 and ending at any one of amino acids 119 to 170 of SEQ ID NO:2, not including the polypeptide having the amino acid sequence of SEQ ID NO:2, and wherein the polypeptide binds a hOX40L or a receptor binding fragment thereof. In some embodiments, a polynucleotide encoding a polypeptide and/or a polypeptide has at least 90% sequence identity to the polypeptide comprising the amino acid sequence of amino acids 29 to 147 of SEQ ID NO:2, not including the polypeptide having the amino acid sequence of SEQ ID NO:2, and wherein the polypeptide binds a hOX40L or a receptor binding fragment thereof. The disclosure also includes a crystalline form of each of these polypeptides.

In some embodiments, a polynucleotide encoding a polypeptide and/or a polypeptide comprises the amino acid sequence of amino acids 35 to 124 of SEQ ID NO:2, not including the polypeptide having the amino acid sequence of SEQ ID NO:2. In some embodiments, a polynucleotide encoding a polypeptide and/or a polypeptide comprises the amino acid sequence of amino acids 31 to 119 of SEQ ID NO:2, not including the polypeptide having the amino acid sequence of SEQ ID NO:2. The present disclosure also includes an isolated polypeptide comprising, consisting essentially of, or consisting of a portion or fragment of the hOX40 receptor. In some embodiments, the polypeptide comprises a binding site for hOX40L and/or mOX40L. In other embodiments, the polypeptide comprises at least CRD1, CRD2 and CRD3. In other embodiments, the polypeptide comprises an extracellular domain of hOX40 receptor. In some embodiments, the polypeptide binds to hOX40L or a receptor binding fragment thereof. The disclosure also includes a crystalline form of each of these polypeptides.

In some embodiments, a mOX40L binding site for hOX40 receptor comprises, consists essentially of, or consists of at least one amino acid residue corresponding to an amino acid selected from the group consisting of R64, A66, T68, S78, Y80, K81, N82, E83, D99, F111, Q112, H119, R121, N125, P126, S145, L146, A147, F148, K149, D150, L166, Q167, I168, N169, G171, Y182, P185, G187, S188, Y189, H190 of the polypeptide comprising SEQ ID NO:1, and combinations thereof. In some embodiments, a mOX40L binding site for hOX40 receptor comprises, consists essentially of, or consists of at least one amino acid residue corresponding to an amino acid selected from the group consisting of A66, T68, Y80, N82, E83, D99, F111, N125, P126, H119, S145 A147, F148, K149, D150, Q167, I168, N169, G171 Y182, S188, Y189 of the polypeptide comprising SEQ ID NO:1, and combinations thereof. In some embodiments, a mOX40L binding site for hOX40 receptor comprises, consists essentially of, or consists of at least one amino acid residue corresponding to an amino acid selected from the group consisting of A66, Y80, D99, F111, A147, N169, Y182, S188 of the polypeptide comprising SEQ ID NO:1 and combinations thereof. In some embodiments, a mOX40L binding site for hOX40 receptor comprises, consists essentially of, or consists of at least one amino acid residue that has at least about 25% of its surface area buried in crystal structure of mOX40L and hOX40 receptor. The disclosure also includes a crystal comprising a crystal structure defined by the structural coordinates of all or a portion of the binding site of mOX40L for hOX40 receptor, as well as the use of the structural coordinates in the methods disclosed herein.

In some embodiments, a hOX40L binding site for hOX40 receptor comprises, consists essentially of, or consists of at least one amino acid residue corresponding to an amino acid selected from the group consisting of Q65, T67, Q80, E82, D98, Y108, F109, S110, Y119, E123, E124, S142, T144, Y145, K146, D147, D162, H164, V165, N166, G167, F180, V182 of the polypeptide comprising SEQ ID NO:3, and combinations thereof. In some embodiments, a hOX40L binding site for hOX40 receptor comprises, consists essentially of, or consists of at least one amino acid residue corresponding to an amino acid selected from the group consisting of Q65, T67, E82, D98, S110, E123, T144, Y145, K146, D147, D162, H164, V165, N166, G167, F180, V182 of the polypeptide comprising SEQ ID NO:3, and combinations thereof. In some embodiments, a hOX40L binding site for hOX40 receptor comprises, consists essentially of, or consists of at least one amino acid residue corresponding to an amino acid selected from the group consisting of D98, T144, D162, N166, F180 of the polypeptide comprising SEQ ID NO:3, and combinations thereof. In some embodiments, a hOX40L binding site for hOX40 receptor comprises, consists essentially of, or consists of at least one amino acid residue that has at least 25% of its surface area buried in crystal structure of hOX40L and hOX40 receptor. The disclosure also includes a crystal comprising a crystal structure defined by the structural coordinates of all or a portion of the binding site of hOX40L for hOX40 receptor, as well as the use of the structural coordinates in the methods disclosed herein.

In some embodiments, a hOX40 receptor binding site for hOX40L comprises, consists essentially of, or consists of at least one amino acid residue corresponding to an amino acid selected from the group consisting of T35, Y36, P37, S38, E45, M52, V53, S54, R55, R65, F71, V75, S78, K79, P80, K82, P83, C84, T85, W86, C87, N88, L89, Y119, K120, V123, D124 of the polypeptide comprising SEQ ID NO:2, and combinations thereof. In some embodiments, a hOX40 receptor binding site for hOX40L comprises, consists essentially of, or consists of at least one amino acid residue corresponding to an amino acid selected from the group consisting of Y36, P37, S38, E45, V53, R55, S78, K79, P83, T85, W86, C87, N88, Y119, V123 of the polypeptide comprising SEQ ID NO:2, and combinations thereof. In some embodiments, a hOX40 receptor binding site for hOX40L comprises, consists essentially of, or consists of at least one amino acid residue corresponding to an amino acid selected from the group consisting of, V53, R55, S78, K79, C87, N88 of the polypeptide comprising SEQ ID NO:2, and combinations thereof. In some embodiments, a hOX40 receptor binding site for hOX40L comprises, consists essentially of, or consists of at least one amino acid residue that has at least 25% of its surface area buried in crystal structure of hOX40L and hOX40 receptor. The disclosure also includes a crystal comprising a crystal structure defined by the structural coordinates of all or a portion of the binding site of hOX40 receptor for hOX40L, as well as the use of the structural coordinates in the methods disclosed herein.

In some embodiments, a hOX40 receptor binding site for mOX40L comprises, consists essentially of, or consists of at least one amino acid residue corresponding to an amino acid selected from the group consisting of L29, H30, C31, G33, T35, Y36, P37, S38, E45, M52, V53, S54, R55, F71, V75, S78, K79, P80, C81, P83, C84, T85, W86, C87, N88, R90, Y119, K120, V123 of the polypeptide comprising SEQ ID NO:2, and combinations thereof. In some embodiments, a hOX40 receptor binding site for mOX40L comprises, consists essentially of, or consists of at least one amino acid residue corresponding to an amino acid selected from the group consisting of C31, G33, T35, Y36, P37, S38, E45, M52, V53, S54, R55, F71, S78, K79, P80, P83, C84, W86, N88, Y119 of the polypeptide comprising SEQ ID NO:2, and combinations thereof. In some embodiments, a hOX40 receptor binding site for mOX40L comprises, consists essentially of, or consists of at least one amino acid residue corresponding to an amino acid selected from the group consisting of C31, Y36, P37, M52, R55, and P80 of the polypeptide comprising SEQ ID NO:2, and combinations thereof. In some embodiments, a hOX40 receptor site for mOX40L comprises, consists essentially of, or consists of at least one amino acid residue that has at least 25% of its surface area buried in crystal structure of mOX40L and hOX40 receptor. The disclosure also includes a crystal comprising a crystal structure defined by the structural coordinates of all or a portion of the binding site of hOX40 receptor for mOX40L, as well as the use of the structural coordinates in the methods disclosed herein.

In some embodiments, a hOX40 receptor binding site for FIV comprises, consists essentially of, or consists of at least one amino acid corresponding to an amino acid selected from the group consisting of R58, S59, N61, H44, V63 of the polypeptide comprising the amino acid sequence of SEQ ID NO:2, and combinations thereof.

Another aspect of the disclosure includes a three-dimensional configuration of points wherein at least a portion of the points are derived from structure coordinates of Table 8 or 9 representing locations of the backbone atoms of amino acids defining the mOX40L binding site for hOX40 receptor. Other embodiments include a three-dimensional configuration of points wherein at least a portion of the points are derived from structure coordinates of Table 10 representing locations of the backbone atoms of amino acids defining the hOX40L binding site for hOX40 receptor. Other embodiments include a three-dimensional configuration of points wherein at least a portion of the points are derived from structure coordinates of Table 10 representing locations of the backbone atoms of amino acids defining the binding site on the hOX40 receptor for hOX40L. Other embodiments include a three-dimensional configuration of points wherein at least a portion of the points are derived from structure coordinates of Table 9 representing locations of the backbone atoms of amino acids defining the binding site on the hOX40 receptor for mOX40L. The three-dimensional configuration of points of can be displayed as a holographic image, a stereodiagram, a model, or a computer-displayed image of at least a portion of the points derived from structure coordinates listed in Tables 8, 9, and/or 10.

Also provided herein is a machine-readable data storage medium comprising a data storage material encoded with machine-readable data, wherein a machine programmed with instructions for using such data displays a graphical three-dimensional representation of at least one molecule or molecular complex comprising at least a portion of a mOX40L binding site for hOX40 receptor, hOX40L binding site for hOX40 receptor, hOX40 receptor binding site for mOX40L, hOX40 receptor binding site for hOX40L, or the binding site defined by a set of points having a root mean square deviation of less than about 0.05 Å from points representing the atoms of the amino acids as represented by the structure coordinates listed in Tables 8, 9, or 10. In some embodiments, the data comprises all or a portion of the diffraction data and/or structural coordinates of the crystals as described herein. In some embodiments, the invention includes a machine readable data storage medium comprising data storage material encoded with a first set of machine readable data which is combined with a second set of machine readable data using a machine programmed with instructions for using the first and second sets of data, and which determines at least a portion of the structure coordinates corresponding to the second set of data, wherein the first set of data comprises a Fourier transform of at least a portion of the structural coordinates of Tables 8, 9, and/or 10, and wherein the second set of data comprises an X-ray diffraction pattern of a molecule or molecular complex for which the three dimensional structure is unknown or incompletely known.

Likewise, the disclosure also includes the scalable three-dimensional configuration of points derived from structure coordinates of molecules or molecular complexes that are structurally homologous to mOX40L, hOX40L, hOX40 receptor or combinations thereof as well as structurally equivalent configurations. Structurally homologous molecules or molecular complexes are defined below. Advantageously, structurally homologous molecules can be identified using all or a portion of the structure coordinates of the Tables 8, 9, and/or 10 according to a method of the disclosure.

Another aspect of the invention provides several different methods. In some embodiments, a computer-assisted method for identifying an agent that modulates mOX40L or hOX40L signaling comprising (a) providing a computer modeling application with a set of structure coordinates of any one of Tables 8, 9, and/or 10 defining at least a portion of a mOX40L binding site, hOX40L binding site, hOX40 receptor binding site for mOX40L, hOX40 receptor binding site for hOX40L, or the binding site defined by a set of points having a root mean square deviation of less than about 0.7 Å from points representing the atoms of the amino acids as represented by the structure coordinates listed in any one of Tables 8, 9, and/or 10; (b) providing the computer modeling application with a set of structure coordinates for a test agent; and (c) modeling the structure of (a) complexed with (b) to determine if the test agent associates with a binding site is provided. The methods of the disclosure further comprise testing the test agent in an assay for binding and/or modulating activity.

Another embodiment includes a computer-assisted method for designing an agent that binds the mOX40L binding site, hOX40L binding site, hOX40 receptor binding site for mOX40L, hOX40 receptor binding site for hOX40L, or the binding site defined by a set of points having a root mean square deviation of less than about 0.7 Å from points representing the atoms of the amino acids as represented by the structure coordinates listed in Tables 8, 9, and/or 10 comprising (a) providing a computer modeling application with a set of structural coordinates of any one of Tables 8, 9, and/or 10 defining at least a portion of the mOX40L binding site, hOX40L binding site, hOX40 receptor binding site for mOX40L, hOX40 receptor binding site for hOX40L, or the binding site defined by a set of points having a root mean square deviation of less than about 0.7 Å from points representing the atoms of the amino acids as represented by the structure coordinates listed in of any one of Tables 8, 9, and/or 10; and (b) modeling the structural coordinates of (a) to identify an agent that contacts at least one amino acid residue in one of the binding site. The method of the disclosure further comprises testing the agent in an assay for binding to mOX40L, hOX40L, hOX40 receptor binding site for mOX40L, and/or hOX40 receptor binding site for hOX40L.

Another embodiment includes a method of identifying a molecule that mimics mOX40L, hOX40L, or hOX40 receptor comprising a) searching a molecular structure database with all or a portion of the structural coordinates of any one of Tables 8, 9, and/or 10; and selecting a molecule from the database that mimics the structural coordinates of the mOX40L, hOX40L, or an hOX40 receptor. The method of the disclosure further comprises testing the molecule in an assay, for example, binding to mOX40L, hOX40L, hOX40 receptor binding site for mOX40L, and/or hOX40 receptor binding site for hOX40L. Another embodiment includes a method of identifying agents that are antagonists or agonists of mOX40L, hOX40L, or an hOX40 receptor comprising a) applying at least a portion of the crystallography coordinates of any one of Tables 8, 9, and/or 10 to a computer algorithm that generates a 3 dimensional model of mOX40L, hOX40L, an hOX40 receptor or combinations thereof suitable for designing molecules that are antagonists or agonists; and b) searching a molecular structure database to identify potential antagonists or agonists of mOX40L, hOX40L, hOX40 receptor or combinations thereof. The methods of the disclosure further comprise testing the agent in an assay for inhibiting or enhancing the activity of mOX40L, hOX40L, hOX40 receptor binding site for mOX40L, and/or hOX40 receptor binding site for hOX40L.

Another embodiment includes a method of assessing agents that are antagonists or agonists of mOX40L, hOX40L, or hOX40 receptor, or combinations thereof, comprising: a) contacting a candidate antagonist or agonist with mOX40L, hOX40L, or hOX40 receptor, or combinations thereof, and selecting the antagonist or agonist that modulates the activity of mOX40L, hOX40L, or hOX40 receptor, or combinations thereof; b) identifying the structure of the selected antagonist or agonist and obtaining the structural coordinates of the selected antagonist or agonist's; c) applying at least a portion of the crystallography coordinates of Tables 8, 9, and/or 10 to a computer algorithm that generates a 3 dimensional model of mOX40L, hOX40L, or hOX40 receptor, or combinations thereof, suitable for designing molecules that are antagonists or agonists to the coordinates of the selected antagonist or agonist; and d) designing a modified antagonist or agonist of the selected antagonist or agonist by performing a fitting operation between the structural coordinates for the selected antagonist or agonist and at least a portion of the structural coordinates of Tables 8, 9, and/or 10. The method of the disclosure further comprises testing the antagonist or agonist in an assay for inhibiting or enhancing the activity of mOX40L, hOX40L, hOX40 receptor binding site for mOX40L, and/or hOX40 receptor binding site for hOX40L.

A further embodiment includes a method for evaluating the ability of a chemical agent to associate with a molecule or molecular complex comprising at least one amino acid residue in mOX40L binding site, hOX40L binding site, hOX40 receptor binding site for mOX40L, hOX40 receptor binding site for hOX40L, or the binding site defined by a set of points having a root mean square deviation of less than about 0.7 Å from points representing the atoms of the amino acids as represented by the structure coordinates listed in Tables 8, 9, and/or 10, said method comprising employing computational means to perform a fitting operation between the chemical agent and the structure coordinates of the amino acid residues of the binding site; and analyzing the results of the fitting operation and selecting those chemical agents that associate with the amino acid residue as defined by favorable polar, nonpolar, electrostatic, shape complementarity, or combinations thereof after conformational adjustments to the binding site. The method of the disclosure further comprises testing the chemical agent in an assay, for example, binding to mOX40L, hOX40L, hOX40 receptor binding site for mOX40L, and/or hOX40 receptor binding site for hOX40L.

### BRIEF DESCRIPTION OF THE DRAWINGS

FIG. 1: The hOX40L trimer differs from other TNFSF members. a) The hOX40L trimers are each rendered as ribbon. Some of the residues along the trimer axis are identified. b) Ribbon rendering of the LT trimer (pdb code: 1tnr) labeled similarly to a.). Note the close packing of the protomers along the trimer axis. c.) LT protomer (gray) is superimposed on one of the hOX40L protomers showing that hOX40L has shorter strands but that the protomer structure is conserved. Approximate hOX40L trimer axis is shown as an arrow. d.) LT trimer (gray) superimposed on hOX40L (black) showing that the trimer assemblies differ. Dashed Black arrow, approximate LT trimer axis. Dark Gray arrow, hOX40L trimer axis.

FIG. 2: The hOX40-hOX40L complex. hOX40L is shown as a molecular surface. The three protomers forming the trimer are shown as light gray, and dark gray. One copy of hOX40 receptor is shown as a tube with CRD1, CRD2, CRD3, and the vestigial CRD4. Disulfide bonds in OX40 receptor are rendered as sticks with the sulfur atoms shown as spheres. The N(29) and C-termini (168) of the receptor are labeled. The other two copies of OX40 receptor are shown with transparent molecular surfaces. There are 7 residues missing from the N-terminus of the ligand which form a linker connecting to the ligand transmembrane domain. These residues are disordered in the electron density and are likely flexible. The complex is oriented such that the membranes of the ligand and receptor containing cells would be at the top and bottom of the figure respectively.

FIG. 3: Structure-based sequence alignment of representatives from the “divergent” (hOX40L), “conventional” (LT; 11% sequence identity with hOX40L) and “EF-disulfide” (BAFF; 10% sequence identity with hOX40L) TNFSF sub-families. Residues forming β-strands are underlined. hOX40L strands are also shown as arrows above the sequences and labeled. Some of the residues which bury at least 50% of accessible surface area and at least 10 A² upon binding receptor are shadowed in gray. hOX40L residues which resulted in at least a 10-fold decrease in IC50 are N166 and F180. OX40L residues which resulted in a 3-fold or greater decrease in IC50 include Q80, E123, T144, D162, N166 and F180. In the LT sequence, the hydrophobic residue (Y108) in the DE loop is conserved in the “conventional” ligands.

FIG. 4: Sequence alignment of CRDs containing B1 modules. a.) Sequence alignments of B1 modules lacking the 4-6 disulfide. The A and B modules are labeled. TNFR1CRD3 is included as a reference of a conventional A1, B2 containing CRD which has been structurally characterized. Cysteine residues are underlined, and the connectivity is indicated by lines above the sequences. b.) Sequence alignment of CRDs containing B1 modules lacking the 3-5 disulfide (the “B2-like” B1 modules). Sequences of TNFR1 CRD2, DR5 CRD2, and OX40 CRD2 are included as examples conventional A1, B2 containing CRD which have been structurally characterized. The aromatic/small residue pair replacing the 3-5 disulfide in the B2-like B1 modules is highlighted in bold.

FIG. 5: Open book view of the hOX40L-hOX40 interface. a.) hOX40L and one receptor are rendered as molecular surfaces. Some of the residues in the interface are shown by % of accessible surface area buried upon complex formation (1-25%, light gray; 25-50%, darker gray; 50-75%, darker gray; 75-100%, black; See Table 6 for residue identification). Potential glyycosylation sites are also shown; N90, N114, N152, and N157. Ligand residues N90 and N114 were mutated to Asp to remove potential glycoylation sites; other glycosylation sites include N152, and N157. Some of the residues of interest are labeled. Underlined residues are identical in murine and human OX40L. b.) hOX40L is rendered as in a.) but with residues with a greater than 10-fold increase or 5-fold increase in IC50 when mutated to alanine colored black and dark gray respectively. Residues which had less than a 5-fold change in IC50 when mutated to alanine have a white background (E123 and T144). c.) shows hOX40 receptor; residues shown with a black background have 75% or greater surface area buried, dark gray have 50% or greater surface area buried. (See Table 6)

### DETAILED DESCRIPTION OF THE PREFERRED EMBODIMENT

### Definitions

The following definitions are used herein, unless otherwise described:

The term “OX40 ligand” or “OX40L”, as used herein, refers, unless specifically or contextually indicated otherwise, to any (whether native or synthetic) OX40L polypeptide that is capable of binding to OX40 receptor and/or activating the OX40 receptor under conditions that permit such process to occur. The term “wild type OX40L sequence” generally refers to an amino acid sequence found in naturally occurring OX40L and includes naturally occurring truncated or secreted forms, variant forms (e.g. alternatively spliced forms) and naturally occurring allelic variants. An example of a wild-type murine OX40L is a polypeptide comprising an amino acid sequence of SEQ ID NO:1 in Table 3. An example of a wild-type murine OX40L is a polypeptide comprising an amino acid sequence of amino acids 51-198 of SEQ ID NO:1 in Table 3b. An example of a wild-type human OX40L is a polypeptide comprising an amino acid sequence of SEQ ID NO:3 in Table 5. An example of a wild-type human OX40L is a polypeptide comprising an amino acid sequence of amino acids 51 to 183 of SEQ ID NO:3 in Table 5b. The sequence numbering of mOX40L or hOX40L begins with the methionine shown as the first amino acid in Table 3 or Table 5, respectively.

“OX40L polypeptides” may also include polypeptides that have a different sequence than a reference polypeptide. Polypeptides can have substitutions, additions or deletions. In some embodiments, the reference polypeptide is a OX40L polypeptide comprising SEQ ID NO:1, comprising SEQ ID NO:3, or fragments thereof. In some embodiments, “non-naturally” occurring variant polypeptides are those prepared synthetically or recombinantly with substitutions, deletions or additions as compared to a naturally occurring sequence. In some embodiments, a variant has at least 80% amino acid sequence identity with the amino acid sequence of SEQ ID NO:1, SEQ ID NO:3, or fragments thereof. In some embodiments, the polypeptides have the biological activity of binding to the hOX40 receptor and/or activating it. In other embodiments, the polypeptide can bind to the hOX40 receptor, but not activate it. Ordinarily, a OX40L polypeptide will have at least 80% sequence identity, more preferably will have at least 81% sequence identity, more preferably will have at least 82% sequence identity, more preferably will have at least 83% sequence identity, more preferably will have at least 84% sequence identity; more preferably will have at least 85% sequence identity, more preferably will have at least 86% sequence identity, more preferably will have at least 87% sequence identity, more preferably will have at least 88% sequence identity, more preferably will have at least 89% sequence identity, more preferably will have at least 90% sequence identity, more preferably will have at least 91% sequence identity, more preferably will have at least 92% sequence identity, more preferably will have at least 93% sequence identity, more preferably will have at least 94% sequence identity, more preferably will have at least 95% sequence identity, more preferably will have at least 96% sequence identity, more preferably will have at least 96% sequence identity, more preferably will have at least 97% sequence identity, more preferably will have at least 98% sequence identity, more preferably will have at least 99% sequence identity with a OX40L polypeptide comprising an amino acid sequence comprising SEQ ID NO:1, comprising SEQ ID NO:3, or fragments thereof. Preferably, the variant polypeptides bind to the hOX40 receptor. Fragments include polypeptides comprising the amino acid sequence of amino acids 51-198 of SEQ ID NO:1 (Table 3b) or comprising the amino acids sequence of amino acids 51-183 of SEQ ID NO:3 (Table 5b). In specific embodiments, a human OX40L polypeptide comprises at least one amino acid substitution such as N90D, N114D, F180A, N166A, Q80A, D162A, T144A, E123A or mixtures thereof.

The term “OX40 receptor”, as used herein, refers to any (whether native or synthetic) OX40 receptor polypeptide that is capable of binding to an OX40L. The term “wild-type OX40 receptor” generally refers to a polypeptide comprising an amino acid sequence found in a naturally occurring receptors and includes naturally occurring truncated or secreted forms, variant forms (e.g. alternatively spliced forms) and naturally occurring allelic variants. An embodiment of the human OX40 receptor has an amino acid sequence of SEQ ID NO:2 shown in Table 4. An embodiment of the human OX40 receptor has an amino acid sequence of amino acid residues of 29 to 170 of SEQ ID NO:2 shown in Table 4b. The sequence numbering of hOX40 receptor begins with the methionine shown as the first amino acid as shown in Table 4.

“OX40 receptor” also refers to a polypeptide that has a different sequence than a reference polypeptide. In some embodiments, the reference polypeptide is an OX40 receptor comprising SEQ ID NO:2. In some embodiments, “non-naturally” occurring variants include those polypeptides that have substitutions, additions or deletions as compared to a wild-type or naturally occurring sequence. In some embodiments, the polypeptide can bind to the OX40L. Ordinarily, a OX40 receptor polypeptide will have at least 80% sequence identity, more preferably will have at least 81% sequence identity, more preferably will have at least 82% sequence identity, more preferably will have at least 83% sequence identity, more preferably will have at least 84% sequence identity; more preferably will have at least 85% sequence identity, more preferably will have at least 86% sequence identity, more preferably will have at least 87% sequence identity, more preferably will have at least 88% sequence identity, more preferably will have at least 89% sequence identity, more preferably will have at least 90% sequence identity, more preferably will have at least 91% sequence identity, more preferably will have at least 92% sequence identity, more preferably will have at least 93% sequence identity, more preferably will have at least 94% sequence identity, more preferably will have at least 95% sequence identity, more preferably will have at least 96% sequence identity, more preferably will have at least 96% sequence identity, more preferably will have at least 97% sequence identity, more preferably will have at least 98% sequence identity, more preferably will have at least 99% sequence identity with a OX40 receptor polypeptide comprising an amino acid sequence comprising SEQ ID NO:2 or a fragment thereof.

The term “binding site,” as used herein, refers to a region of a molecule or molecular complex that, as a result of its shape, distribution of electrostatic charge, presentation of hydrogen-bond acceptors or hydrogen-bond donors, and/or distribution of nonpolar regions, favorably associates with a ligand. Thus, a binding site may include or consist of features such as cavities, surfaces, or interfaces between domains. Ligands that may associate with a binding site include, but are not limited to, cofactors, substrates, receptors, agonists, and antagonists. The term binding site includes a functional binding site and/or a structural binding site. A structural binding site can include “in contact” amino acid residues as determined from examination of a three-dimensional structure. “Contact” can be determined using Van der Waals radii of atoms or by proximity sufficient to exclude solvent, typically water, from the space between the ligand and the molecule or molecular complex. In some embodiments, a OX40L residue in contact with OX40 receptor or other substrate or inhibitor is a residue that has one atom within about 5 Å of a hOX40 receptor residue. Alternatively, “in contact” residue may be those that have a loss of solvent accessible surface area of at least about 25% to 100%, more preferably at least about 50% to 100%, more preferably about 75% to 100% and/or at least about 10 angstroms squared of surface area is lost. In some embodiments, loss of solvent accessible surface determinations can vary plus or minus 5%. Loss of solvent accessible surface can be determined by the method of Lee & Richards (J Mol Biol, 55(3):379-400 (1971)) and similar algorithms known to those skilled in the art, for instance as found in the SOLV module from C. Broger of F. Hoffman-La Roche in Basel Switzerland.

Some of the “in contact” amino acid residues, if substituted with another amino acid type, may not cause any change in a biochemical assay, a cell-based assay, or an in vivo assay used to define a functional binding site but may contribute to the formation of a three dimensional structure. A functional binding site includes amino acid residues that are identified as binding site residues based upon loss or gain of function, for example, loss of binding to ligand upon mutation of the residue. In some embodiments, the amino acid residues of a functional binding site are a subset of the amino acid residues of the structural binding site.

The term “hOX40L binding site” refers to a region of a human OX40L that can favorably associate with a hOX40 receptor.

The term “mOX40L binding site” refers to a region of a murine OX40L that can favorably associate with a hOX40 receptor.

The term “hOX40 receptor binding site” refers to a region of a human OX40 receptor that can favorably associate with a OX40L, such as mOX40L and/or hOX40L.

A structurally equivalent ligand binding site is defined by a root mean square deviation from the structure coordinates of the backbone atoms of the amino acids that make up a binding sites as described herein of at most about 0.70 Å, preferably about 0.5 Å.

“Crystal” as used herein, refers to one form of a solid state of matter in which atoms are arranged in a pattern that repeats periodically in three-dimensions, typically forming a lattice.

“Complementary or complement” as used herein, means the fit or relationship between two molecules that permits interaction, including for example, space, charge, three-dimensional configuration, and the like.

The term “corresponding” or “corresponds” refers to an amino acid residue or amino acid sequence that is found at the same position or positions in a sequence when the amino acid position or sequences are aligned with a reference sequence to maximize sequence identity. In some embodiments, the reference sequence is a fragment of the mOX40L having a sequence of amino acids residues 51-198 of SEQ ID NO:1. In some embodiments, the reference sequence is a fragment of the hOX40 receptor having a sequence of amino acids residues 29-170 of SEQ ID NO:2. In some embodiments, the reference sequence is a fragment of the hOX40L having a sequence of amino acids residues 51-183 of SEQ ID NO:3. It will be appreciated that when the amino acid position or sequence is aligned with the reference sequence the numbering of the amino acids may differ from that of the reference sequence.

“Heavy atom derivative”, as used herein, means a derivative produced by chemically modifying a crystal with a heavy atom such as Hg, Au, or a halogen.

“Structural homolog” of OX40L or hOX40 receptor as used herein refers to a protein that contains one or more amino acid substitutions, deletions, additions, or rearrangements with respect to the amino acid sequence of OX40L or hOX40 receptor, but that, when folded into its native conformation, exhibits or is reasonably expected to exhibit at least a portion of the tertiary (three-dimensional) structure of the OX40L or hOX40 receptor.

Tertiary structure can be probed, measured, or confirmed by known analytic or diagnostic methods, for example, X-ray, NMR, circular dichroism, a panel of monoclonal antibodies that recognize OX40L, hOX40 receptor or complexes thereof and like techniques. For example, structurally homologous molecules can have substitutions, deletions or additions of one or more contiguous or noncontiguous amino acids, such as a loop or a domain. Structurally homologous molecules also include “modified” OX40L or hOX40 receptor molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and like modifications.

“Ligand”, as used herein, refers to an agent or compound that associates with a binding site on a molecule, for example, OX40L binding sites for hOX40 receptor, and may be an antagonist or agonist of OX40L activity. Ligands include molecules that mimic OX40L binding to hOX40 receptor and in some embodiments, are not capable of activating hOX40 receptor.

“Compound” refers to molecule that associates with the hOX40 receptor, mOX40L, hOX40L, or complexes thereof or a pharmaceutically acceptable salt, ester, amide, prodrug, isomer, or metabolite, thereof “Pharmaceutically acceptable salt” refers to a formulation of a compound that does not compromise the biological activity and properties of the compound. Pharmaceutical salts can be obtained by reacting a binding-active compound of the disclosure with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. “Prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transport across a cell membrane where water solubility is detrimental to mobility but which then is metabolically hydrolyzed to the carboxylic acid, the active entity, once inside the cell where water solubility is beneficial. A further example of a prodrug might be a short peptide (polyaminoacid) bonded to an acid group wherein the peptide is metabolized to yield the active moiety.

“Molecular complex”, as used herein, refers to a combination of bound substrate or ligand with polypeptide, such as OX40L bound to hOX40 receptor, or a ligand bound to an hOX40 receptor and/or OX40L.

“Machine-readable data storage medium”, as used herein, means a data storage material encoded with machine-readable data, wherein a machine programmed with instructions for using such data and is capable of displaying data in the desired format, for example, a graphical three-dimensional representation of molecules or molecular complexes.

“Scalable,” as used herein, means the increasing or decreasing of distances between coordinates (configuration of points) by a scalar factor while keeping the angles essentially the same.

“Space group symmetry”, as used herein, means the whole symmetry of the crystal that combines the translational symmetry of a crystalline lattice with the point group symmetry. A space group is designated by a capital letter identifying the lattice type (P, A, F, etc.) followed by the point group symbol in which the rotation and reflection elements are extended to include screw axes and glide planes. Note that the point group symmetry for a given space group can be determined by removing the cell centering symbol of the space group and replacing all screw axes by similar rotation axes and replacing all glide planes with mirror planes. The point group symmetry for a space group describes the true symmetry of its reciprocal lattice.

“Unit cell”, as used herein, means the atoms in a crystal that are arranged in a regular repeating pattern, in which the smallest repeating unit is called the unit cell. The entire structure can be reconstructed from knowledge of the unit cell, which is characterized by three lengths (a, b and c) and three angles (α, β and γ). The quantities a and b are the lengths of the sides of the base of the cell and γ is the angle between these two sides. The quantity c is the height of the unit cell. In some embodiments, the unit cell lengths can vary plus or minus 0.5 Å. The angles α and β describe the angles between the base and the vertical sides of the unit cell.

“X-ray diffraction pattern” means the pattern obtained from X-ray scattering of the periodic assembly of molecules or atoms in a crystal. X-ray crystallography is a technique that exploits the fact that X-rays are diffracted by crystals. X-rays have the proper wavelength (in the Ångstrom (Å) range, approximately 10⁻⁸ cm) to be scattered by the electron cloud of an atom of comparable size. Based on the diffraction pattern obtained from X-ray scattering of the periodic assembly of molecules or atoms in the crystal, the electron density can be reconstructed. Additional phase information can be extracted either from the diffraction data or from supplementing diffraction experiments to complete the reconstruction (the phase problem in crystallography). A model is then progressively built into the experimental electron density, refined against the data to produce an accurate molecular structure.

X-ray structure coordinates define a unique configuration of points in space. Those of skill in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a configuration of points can be defined by increasing or decreasing the distances between coordinates by a scalar factor, while keeping the angles essentially the same.

“Crystal structure” generally refers to the three-dimensional or lattice spacing arrangement of repeating atomic or molecular units in a crystalline material. The crystal structure of a crystalline material can be determined by X-ray crystallographic methods, see for example, “Principles of Protein X-Ray Crystallography,” by Jan Drenth, Springer Advanced Texts in Chemistry, Springer Verlag; 2nd ed., February 1999, ISBN: 0387985875, and “Introduction to Macromolecular Crystallography,” by Alexander McPherson, Wiley-Liss, Oct. 18, 2002, ISBN: 0471251224.

### MODES FOR CARRYING OUT THE INVENTION

The present disclosure thus includes a crystalline form and a crystal structure of each of a murine OX40L (mOX40L) and a mOX40L or a hOX40L complexed with a hOX40 receptor. In other aspects, the disclosure provides methods of using the crystal structures and structural coordinates to identify homologous proteins and to design or identify agents that can modulate the function of the OX40L, hOX40 receptor and/or complexes thereof. The present disclosure also includes the three-dimensional configuration of points derived from the structure coordinates of at least a portion of a OX40L molecule, hOX40 receptor or molecular complexes thereof, as well as structurally equivalent configurations, as described below. The three-dimensional configuration includes points derived from structure coordinates representing the locations of one or more of a plurality of the amino acids defining the mOX40L binding site when it is not bound to hOX40 receptor, mOX40L binding site when it is bound to hOX40 receptor, hOX40L binding site when it is bound to hOX40 receptor, hOX40 receptor binding site for hOX40L or mOX40L.

In some embodiments, the three-dimensional configuration includes points derived from structure coordinates representing the locations of the backbone atoms of a plurality of amino acids defining the defining the mOX40L binding site when it is not bound to hOX40 receptor, mOX40L binding site when it is bound to hOX40 receptor, hOX40L binding site when it is bound to hOX40 receptor, and hOX40 receptor binding site for hOX40L or mOX40L. Alternatively, the three-dimensional configuration includes points derived from structure coordinates representing the locations of the side chain and the backbone atoms (other than hydrogens) of a plurality of the amino acids defining the defining the mOX40L binding site when it is not bound to hOX40 receptor, mOX40L binding site when it is bound to hOX40 receptor, hOX40L binding site when it is bound to hOX40 receptor, and hOX40 receptor binding site for hOX40L or mOX40L.

Likewise, the disclosure also includes the scalable three-dimensional configuration of points derived from structure coordinates of molecules or molecular complexes that are structurally homologous to OX40L, hOX40 receptor, or complexes thereof as well as structurally equivalent configurations. Structurally homologous molecules or molecular complexes are defined below. Advantageously, structurally homologous molecules can be identified using the structure coordinates of the OX40L, hOX40 receptor, or complexes thereof according to a method of the disclosure.

The configurations of points in space derived from structure coordinates according to the disclosure can be visualized as, for example, a holographic image, a stereodiagram, a model, or a computer-displayed image, and the disclosure thus includes such images, diagrams or models.

The crystal structure and structural coordinates can be used in methods, for example, for obtaining structural information of a related molecule, and for identifying and designing agents that modulate activity or binding of OX40L, hOX40 receptor, or complexes thereof.

The coordinates of mOX40L are provided in Table 8. The coordinates of mOX40L cocrystallized with hOX40 receptor are provided in Table 9. The coordinates of hOX40L cocrystallized with hOX40 receptor are provided in Table 10.

1. OX40L and hOX40 Receptor Polypeptides, Polynucleotides and Variants Thereof.

The present disclosure includes OX40L polypeptides and hOX40 receptor polypeptide.

Native or wild-type OX40L are those polypeptides that have a sequence of a polypeptide obtained from nature. Native or wild-type polypeptides include naturally occurring variants, secreted or truncated forms. An embodiment of wild type murine OX40L comprises a sequence of SEQ ID NO:1 shown in Table 3. An embodiment of wild type human OX40L comprises a sequence of SEQ ID NO:3 shown in Table 5.

In some embodiments, a OX40L includes several domains including A, A″, B′, B, C, D, E, F, G, and H β sheet strands that form jelly roll β sandwich monomer sheets as follows: A′AHCF and B′BGDE. The OX40L forms a flower like trimer with an interface formed by a layer of generally hydrophobic residues along the C strand, F strand, and C terminal extension (amino acid residues 175-183 in hOX40L and residues 178-191 in mOX40L). The monomers are splayed out and form an angle of about 45° with respect to the trimer axis. The trimer interface is compact with about 2600 Å accessible surface area buried.

In another aspect, the disclosure includes mOX40L polypeptides and polynucleotides encoding the polypeptides. The disclosure includes a polynucleotide encoding a polypeptide and/or a polypeptide having at least 90% sequence identity to the polypeptide comprising the amino acid sequence of amino acids 51-198 of SEQ ID NO:1, not including the polypeptide having the amino acid sequence of SEQ ID NO:1 or SEQ ID NO:3, and wherein the polypeptide a hOX40 receptor or a hOX40 receptor ligand binding fragment thereof. Another embodiment, comprises a polynucleotide encoding a polypeptide and/or a polypeptide that comprises the amino acid sequence starting at any one of amino acids 51 to amino acid residue 66 and ending at any one of amino acids 180 to 191 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 64 to 190 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 99 to 191 of SEQ ID NO:1, not including the polypeptide having the amino acid sequence of SEQ ID NO:1 or SEQ ID NO:3, and wherein the polypeptide binds a hOX40 receptor or a hOX40 receptor ligand binding fragment thereof. The disclosure also includes a crystalline form of each of these polypeptides.

The present disclosure also includes an isolated polypeptide comprising, consisting essentially of, or consisting of a portion or fragment of the mOX40L. In some embodiments, the polypeptide comprises a binding site for hOX40 receptor. In some embodiments, the polypeptide comprises an extracellular domain of mOX40L. In other embodiments, the polypeptide comprises the trimer interface. In some embodiments, the trimer interface includes that amino acid residues corresponding to amino acids 99 to 191 of SEQ. ID NO:1. An embodiment of a polypeptide comprises, consists essentially of, or consists of an amino acid residue starting from any one of amino acid residue 51 to amino acid residue 66 and ending at amino acid residue 180 to residue 191 or residues corresponding to those positions in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 51 to 198 in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 66 to 190 in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 64 to 190 in the polypeptide comprising SEQ ID NO:1, excluding the amino acid sequence of SEQ ID NO:1 excluding the amino acid sequence of SEQ ID NO:1. An embodiment of a polypeptide comprises, consists essentially of, or consists of an amino acid residues corresponding to amino acid residues 66 to 189 in the polypeptide comprising SEQ ID NO:1, excluding the amino acid sequence of SEQ ID NO:1. An embodiment of a polypeptide comprises, consists essentially of, or consists of an amino acid residues corresponding to amino acid residues 99 to 191 in the polypeptide comprising SEQ ID NO:1, excluding the amino acid sequence of SEQ ID NO:1. In some embodiments, the polypeptide portion has the ability to bind to hOX40 receptor or a hOX40 receptor ligand binding fragment thereof. The disclosure also includes polynucleotides encoding such polypeptides. The disclosure includes a crystalline form of each of these polypeptides, as well as one structural coordinate and use all or a portion of structural coordinates in the methods described herein.

The disclosure also includes a polynucleotide encoding a polypeptide and/or a polypeptide having at least 90% sequence identity to the polypeptide comprising the amino acid sequence of amino acids 51 to 183 of SEQ ID NO:3, not including the polypeptide having the amino acid sequence of SEQ ID NO:3 or SEQ ID NO:1, and wherein the polypeptide binds a hOX40 receptor or a hOX40 receptor ligand binding fragment thereof. Another embodiment, comprises a polynucleotide encoding a polypeptide and/or a polypeptide that comprises the amino acid sequence starting at any one of amino acids 51 to amino acid residue 65 and ending at any one of amino acids 180 to 183 of SEQ ID NO:3, not including the polypeptide having the amino acid sequence of SEQ ID NO:3 or SEQ ID NO:1, and wherein the polypeptide binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 98 to 183 of SEQ ID NO:3, not including the polypeptide having the amino acid sequence of SEQ ID NO:3 or SEQ ID NO:1, and wherein the polypeptide ity binds a hOX40 receptor or a hOX40 receptor ligand binding fragment thereof. In some embodiments, a polynucleotide encoding a polypeptide and/or a polypeptide has at least 90% sequence identity to the polypeptide comprising the amino acid sequence of amino acids 65 to 182 of SEQ ID NO:3, not including the polypeptide having the amino acid sequence of SEQ ID NO:3 or SEQ ID NO:1, and wherein the polypeptide binds a hOX40 receptor or a hOX40 receptor ligand binding fragment thereof. The disclosure also includes a crystalline form of each of these polypeptides.

In another aspect, the disclosure includes hOX40L polypeptides and polynucleotides encoding the polypeptides. The present disclosure also includes an isolated polypeptide comprising, consisting essentially of, or consisting of a portion or fragment of the hOX40L. In some embodiments, the polypeptide comprises a binding site for hOX40 receptor. In some embodiments, the polypeptide comprises an extracellular domain of hOX40L. In other embodiments, the polypeptide comprises the trimer interface. In some embodiments, the trimer interface includes amino acid residues corresponding to amino acids 98 to 182 or 183 of the amino acid sequence of SEQ ID NO:3. An embodiment of a polypeptide comprises, consists essentially of, or consists of an amino acid residue starting from any one of amino acid residue 51 to amino acid residue 65 and ending at amino acid residue 180 to residue 183 or residues corresponding to those positions in the polypeptide comprising SEQ ED NO:3, excluding the amino acid sequence of SEQ ID NO:3. An embodiment of a polypeptide comprises, consists essentially of, or consists of an amino acid residues corresponding to amino acid residues 58 to 183 in the polypeptide comprising SEQ ID NO:3, excluding the amino acid sequence of SEQ ID NO:3. An embodiment of a polypeptide comprises, consists essentially of, or consists of an amino acid residues corresponding to amino acid residues 65 to 182 in the polypeptide comprising SEQ ID NO:3, excluding the amino acid sequence of SEQ ID NO:3. An embodiment of a polypeptide comprises, consists essentially of, or consists of an amino acid residues corresponding to amino acid residues 98 to 183 in the polypeptide comprising SEQ ED NO:3, excluding the amino acid sequence of SEQ ID NO:3. In some embodiments, the polypeptide portion has the ability to bind to hOX40 receptor and/or hOX40 receptor ligand binding fragment thereof. The disclosure also includes polynucleotides encoding such polypeptides. The disclosure also includes a crystalline form of each of these polypeptides, as well as the structural coordinates and use of all or portion of the coordinates in the methods described herein.

The present disclosure also includes OX40L polypeptides that have amino acid substitutions, deletions, and additions. Amino acid substitutions can be made for example to replace cysteines and eliminate formation of disulfide bonds. Amino acid substitutions can also be made to change proteolytic cleavage sites or eliminate glycosylation sites. Other variants can be made at the OX40L binding site for hOX40 receptor. In other embodiments, the OX40L polypeptides bind hOX40 receptor with the same or higher affinity than a wild type OX40L. In specific embodiments, a human OX40L polypeptide comprises at least one amino acid substitution such as N90D, N114D, F180A, N166A, Q80A, D162A, T144A, E123A or mixtures thereof.

Native or wild type hOX40 receptors are those polypeptides that have a sequence of a polypeptide obtained from nature. A specific embodiment of a human OX40 receptor comprises a sequence of SEQ ID NO:2 as shown in Table 4.

Wild type OX40 receptors are integral cell surface proteins that are composed of three full CRDs and a partial C-terminal CRD which form a contiguous structure. Superposition of the three independent copies of hOX40 (two in the mOX40L-hOX40 asymmetric unit and one in the hOX40L-OX40 asymmetric unit) reveals that CRD1 and CRD2 form a rigid unit while there is some rotational freedom between the subdomains of CRD3 and the partial CRD4. CRD3 in hOX40 differs more than CRD1 and CRD2 from its counterpart in TNFR1 or DR5 as it is smaller and lacks one of the canonical disulfides. CRD3 contains an A1, B1 module rather than the A1, B2 architecture of CRD1 and 2. In the case of OX40, the disulfide formed by the fourth and sixth cysteines in CRD3 (the 4-6 disulfide) is missing. This missing disulfide in hOX40 CRD3 is not replaced by either hydrogen bonds or hydrophobic interactions but rather the entire module is smaller due to shortening and re-arrangement of intervening loops. This re-arrangement causes the backbone of the remaining 3-5 disulfide to be in an anti-parallel orientation rather than the parallel orientation seen in B2 modules. A long disulfide containing loop follows the B1 module. The connectivity and structure of this loop are compatible with it being an A1 module of a vestigial CRD4.

The present disclosure also includes a polypeptide comprising, consisting essentially of, or consisting of a portion or fragment of hOX40 receptor. In some embodiments, the polypeptide comprises an extracellular domain of hOX40L receptor. In other embodiments, the polypeptide comprises a binding site for mOX40L and/or hOX40L. Preferably, the polypeptide comprises one or more of the CRD domains, preferably the CRD1, CRD2, and/or CRD3 domains. In some embodiments, CRD1 comprises amino acids 29-65; CRD2 comprises amino acids 66-108; CRD3 comprises amino acids 109-147; of the amino acid sequence of SEQ ID NO:2. In some embodiments, CRD1 includes at least residues 31-64 including Cys 31, 42, 43, 56, 46 and 64; CRD2 includes at least residues 67-107 including Cys 67, Cys 81, Cys 84, Cys 99, Cys 87 and Cys 107; CRD3 includes at least 109-141 including cys 109, cys 125, cys 128, and cys 141; and CRD4 includes at least residues 147-166 including cys 147 and cys 166 of the amino acid sequence of SEQ ID NO:2.

In another aspect, the disclosure includes hOX40 receptor polypeptides and polynucleotides encoding the polypeptides. The disclosure includes a polynucleotide encoding a polypeptide and/or a polypeptide having at least 90% sequence identity to the polypeptide comprising the amino acid sequence of amino acids 29 to 170 of SEQ ID NO:2, not including the polypeptide having the amino acid sequence of SEQ ID NO:2, and wherein the polypeptide hOX40L, mOX40L, or receptor binding fragment thereof. Another embodiment, comprises a polynucleotide encoding a polypeptide and/or a polypeptide that comprises the amino acid sequence starting at any one of amino acids 29 to amino acid residue 36 and ending at any one of amino acids 119 to 170 of SEQ ID NO:2, not including the polypeptide having the amino acid sequence of SEQ ID NO:2, and wherein the polypeptide binds a hOX40L, mOX40L, or receptor binding fragment thereof. In some embodiments, a polynucleotide encoding a polypeptide and/or a polypeptide has at least 90% sequence identity to the polypeptide comprising the amino acid sequence of amino acids 29 to 147 of SEQ ID NO:2, not including the polypeptide having the amino acid sequence of SEQ ID NO:2, and wherein the polypeptide binds a hOX40L, mOX40L, or receptor binding fragment thereof. The disclosure also includes a crystalline form of each of these polypeptides.

An embodiment of a polypeptide comprises a polypeptide starting at any one of amino acid residues 29 to amino acid residue 36 and ending at any one of amino acid residues 147 to amino acid residue 170 of SEQ ID NO:2, not including the amino acid sequence of SEQ ID NO:2. An embodiment of a polypeptide comprises a polypeptide starting at any one of amino acid residues 31 to amino acid residue 36 and ending at any one of amino acid residues 147 to amino acid residue 170 of SEQ ID NO:2, not including the amino acid sequence of SEQ ID NO:2. An embodiment of a polypeptide comprises a polypeptide starting at any one of amino acid residues 31 to amino acid residue 36 and ending at any one of amino acid residues 119 to amino acid residue 170 of SEQ ID NO:2, not including the amino acid sequence of SEQ ID NO:2. In some embodiments, a polypeptide comprises the amino acid sequence of amino acids 35 to 124 of SEQ ID NO:2, not including the polypeptide having the amino acid sequence of SEQ ID NO:2. In some embodiments, a polypeptide comprises the amino acid sequence of amino acids 31 to 119 of SEQ ID NO:2, not including the polypeptide having the amino acid sequence of SEQ ID NO:2. The fragment preferably comprises a binding site for mOX40L and/or hOX40L. The disclosure also includes polynucleotides encoding these polypeptides. The disclosure also includes a crystalline form of each of these polypeptides, as well as all or a portion of the structural coordinates for use in the methods described herein.

The present disclosure also include hOX40 receptor polypeptides that have amino acid substitutions, deletions, and additions. Amino acid substitutions can be made for example to replace cysteines and eliminate formation of disulfide bonds. Amino acid substitutions can also be made to change proteolytic cleavage sites or eliminate glycosylation sites. Other variants can be made at the amino acid residue or residues that bind to hOX40L and/or mOX40L as identified herein.

### Fusion Proteins

OX40L and/or OX40 receptor polypeptides, structural homologs, or portions thereof, may be fused to a heterologous polypeptide or compound. The heterologous polypeptide is a polypeptide that has a different function than that of the OX40L and/or OX40 receptor. Examples of heterologous polypeptide include polypeptides that may act as carriers, may extend half life, may act as epitope tags, provide for secretion, may provide ways to detect or purify the fusion protein. Heterologous polypeptides include KLH, albumin, salvage receptor binding epitopes, immunoglobulin constant regions, and peptide tags. In a specific embodiment, the polypeptides may be fused to the signal peptide of the 67 kd envelope glycoprotein of AcNPV baculovirus to provide for secretion from cells. Peptide tags useful for detection or purification include FLAG, gD protein, polyhistidine tags, hemagluthinin from influenza virus, T7 tag, S tag, Strep tag, chloramiphenicol acetyl transferase, biotin, glutathione-S transferase, green fluorescent protein and maltose binding protein. Compounds that can be combined with the OX40L, OX40 receptors, variants or structural homolog or portions thereof, include radioactive labels, protecting groups, and carbohydrate or lipid moieties.

### Polynucleotides, Vectors and Host Cells

OX40L, OX40 receptors, variants or fragments thereof can be prepared by introducing appropriate nucleotide changes into DNA encoding OX40L or OX40 receptor, or by synthesis of the desired polypeptide variants.

Polynucleotide sequences encoding mOX40L, hOX40L and hOX40 receptor are known to those of skill in the art and can be obtained from sources such as Genbank and the like. For example, a polynucleotide sequence encoding mOX40L can be found at accession number U12763(gI:551080); an amino acid sequence for mOX40L can be found at accession number P43488; a polynucleotide sequence encoding hOX40L can be found at accession number D90224(gI:219665); an amino acid sequence for hOX40L can be found at accession number P23510; a polynucleotide sequence encoding hOX40 receptor can be found at accession number X65962(gI:472957); and an amino acid sequence for hOX40 receptor can be found at accession number P42489.

Polynucleotide sequences encoding the polypeptides described herein can be obtained using standard recombinant techniques. Desired polynucleotide sequences may be isolated and sequenced from appropriate source cells. Alternatively, polynucleotides can be synthesized using nucleotide synthesizer or PCR techniques. Once obtained, sequences encoding the polypeptides or variant polypeptides are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences, which are derived from a species compatible with the host cell are used in connection with these hosts. The vector ordinarily carries a replication site, as well as marking sequences, which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins.

In addition, phage vectors containing replicon and control sequences that are compatible with the host microorganism can be used as transforming vectors in connection with these hosts. For example, bacteriophage such as γGEM.TM.-11 may be utilized in making a recombinant vector which can be used to transform susceptible host cells such as E. coli LE392.

Either constitutive or inducible promoters can be used in the present invention, in accordance with the needs of a particular situation, which can be ascertained by one skilled in the art. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of the target genes. However, heterologous promoters are preferred, as they generally permit greater transcription and higher yields of expressed target gene as compared to the native target polypeptide promoter.

Promoters suitable for use with prokaryotic hosts include the PhoA promoter, the β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the polypeptides or variant polypeptides (Siebenlist et al., Cell, 20: 269 (1980)) using linkers or adaptors to supply any required restriction sites.

In embodiments, each cistron within a recombinant vector comprises a secretion signal sequence component that directs translocation of the expressed polypeptides across a membrane. In general, the signal sequence may be a component of the vector, or it may be a part of the polypeptide encoding DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

Prokaryotic host cells suitable for expressing polypeptides include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. Preferably, gram-negative cells are used. Preferably the host cell should secrete minimal amounts of proteolytic enzymes, and additional protease inhibitors may desirably be incorporated in the cell culture.

Besides prokaryotic host cells, eukaryotic host cell systems are also well established in the art. Examples of invertebrate cells include insect cells such as Drosophila S2 and Spodoptera Sf9, as well as plants and plant cells. Examples of useful mammalian host cell lines include Chinese hamster ovary (CHO) and COS cells. More specific examples include monkey kidney CV1 line transformed by SV40 (COS-7, ATCC CRL 1651); Chinese hamster ovary cells/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); High Five cells (derived from Trichopulsia ni cell line; High Five cells are available from Invitrogen) and mouse mammary tumor (MMT 060562, ATCC CCL51).

In a specific embodiment, a baculovirus transfer vector is utilized. Several such transfer vectors are known to those of skill in the art and commercially available. For example, BD Pharmingen has several vectors using sequences from AcPNV baculovirus.

### Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the polypeptides of the invention are grown in media known in the art and suitable for culture of the selected host cells. Examples of suitable media include luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector, protein expression is induced under conditions suitable for the activation of the promoter. For example, if a PhoA promoter is used for controlling transcription, the transformed host cells may be cultured in a phosphate-limiting medium for induction. A variety of other inducers may be used, according to the vector construct employed, as is known in the art.

Eukaryotic host cells are cultured under conditions suitable for expression of the OX40L and/or OX40 receptor polypeptides. The host cells used to produce the polypeptides may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in one or more of Ham et al., Meth. Enz., 58:44 (1979), Barnes et al., Anal. Biochem., 102: 255 (1980), U.S. Pat. No. 4,767,704, U.S. Pat. No. 4,657,866, U.S. Pat. No. 4,927,762, U.S. Pat. No. 4,560,655, or U.S. Pat. No. 5,122,469, WO 90/103430, WO 87/00195, and U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES™), nucleotides (such as adenosine and thymidine), antibiotics (such as GENTAMYCIN™), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptides described herein expressed in a host cell may be secreted and/or recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported or secreted into the culture media and isolated there from. Cells may be removed from the culture and the culture supernatant filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Polypeptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

2. Crystals and Crystal Structures

The present disclosure provides a crystalline form of and a crystal structure of the mOX40L, and a crystalline form of and the crystal structures of the hOX40 receptor cocrystallized with either mOX40L or hOX40L. The crystals are formed by contacting a mixture of purified OX40L and/or fragment thereof and/or an hOX40 receptor and/or fragment thereof with a precipitant in a buffer. In some embodiments, the precipitant is about 8-10% polyethylene glycol 20,000. In other embodiments, the precipitant is about 1-2M ammonium sulfate.

mOX40L can be purified and crystallized. In a specific embodiment, m OX40L is a fragment comprising the amino acid sequence of S51 to L198 of SEQ ID NO:1 (Table 3b). The crystals of m mOX40L can be diffracted to about 1.45-2.5 Å resolution (Table 1). The crystals belong in space group P6₃ with a=b and are about 74 Å, and c about 48 Å. Unit cell volume and molecular weight suggested 1 protomer in the asymetric unit. When the crystals are resolubilized, the mOX40L has at least one biological activity. Crystals can be combined with a carrier to form a composition. Crystal of mOX40L may also be a useful way to store, concentrate or deliver mOX40L. Constituent amino acids in mOX40L have a set of structural coordinates as provided in Table 8. NAG indicates carbohydrate residues.

The cocrystals of mOX40L and hOX40 receptor diffracted to about 2.00-2.07 Å resolution (Table 1) and the biologically relevant complex contains 3 ligands forming a trimer and 3 receptors. The structural coordinates for the 2 ligand/receptor pairs in the mOX40L-hOX40 structure are part of two different complexes. In a specific embodiment, mOX40L is a fragment comprising residues S51 to L198 of SEQ ID NO:1 (Table 3b) and hOX40 receptor is a fragment comprising the amino acid sequence of 29-170 of SEQ ID NO:2 (Table 4b).

In a specific embodiment, the structure of mOX40L and hOX40 receptor was solved by molecular replacement with the program AMORE (NAVAZA 1994) using the crystal structure of mOX40L alone as search model. The crystals belonged to space group R32 with cell parameters of: a=b and are about 105 Å, and c is about 478 Å. Crystals of the complex can be combined with a carrier to form a composition. Crystals may also be a useful way to store, concentrate or deliver a of mOX40L and/or hOX40 receptor. Constituent amino acids in mOX40L and/or hOX40 receptor complex have a set of structural coordinates as set forth in Table 9. The coordinates of two molecules of each of mOX40L and hOX40 receptor are provided. The structural coordinates for the 2 ligand/receptor pairs in the mOX40L-hOX40 structure are part of two different complexes. The structural coordinates from each of the molecules as shown in Table 9 may be utilized.

The crystals of hOX40L and hOX40 receptor diffracted to about 2.40-2.49 Å resolution (Table 1) and have one (1) full complex containing one trimeric ligand and 1 receptor in the crystallographic asymmetric unit. Crystallographic three-fold symmetry generates the biologically relevant trimers. In a specific embodiment, hOX40L is a fragment comprising residues 51-183 of SEQ ID NO:3 (Table 5b) and hOX40 receptor is a fragment comprising the amino acid sequence of 29-170 of SEQ ID NO:2 (Table 4b). In some embodiments, hOX40L is a fragment comprising residues 51-183 of SEQ ID NO:3 (Table 5b) with an amino acid substitution N90D and N114D.

In a specific embodiment, the structure of hOX40L and hOX40 receptor was solved by molecular replacement with the program AMORE (NAVAZA 1994) using the crystal structure of mOX40L alone as search model. The crystals belonged to space group R32 with cell parameters of: a=b and are about 112 Å, c is about 233 Å. Crystals of the complex can be combined with a carrier to form a composition. Crystals may also be a useful way to store, concentrate or deliver a complex of hOX40L and/or hOX40 receptor. Constituent amino acids in hOX40L and hOX40 receptor complex have a set of structural coordinates as set forth in Table 10.

The term “structure coordinates” refers to Cartesian coordinates derived from mathematical equations related to the patterns obtained on diffraction of a monochromatic beam of X-rays by the atoms (scattering centers) of a mOX40L, mOX40L-hOX40 receptor complex, or hOX40L-hOX40 receptor in crystal form. The diffraction data are used to calculate an electron density map of the repeating unit of the crystal. The electron density maps are then used to establish the positions of the individual atoms of the mOX40L, mOX40L-hOX40 receptor complex, or hOX40L-hOX40 receptor complex.

Slight variations in structure coordinates can be generated by mathematically manipulating the mOX40L, mOX40L-hOX40 receptor complex, or hOX40L-hOX40 receptor complex structure coordinates. For example, the structure coordinates as set forth in Tables 8, 9 and/or 10 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, deletions, and combinations thereof, of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the mOX40L, mOX40L-OX40 receptor complex, or hOX40L-hOX40 receptor complex would not be expected to significantly alter the nature of chemical entities such as ligands that could associate with a binding site or other structural features of mOX40L, hOX40L, or hOX40 receptor or complexes thereof. In this context, the phrase “associating with” refers to a condition of proximity between a ligand, or portions thereof, and a mOX40L, hOX40L, hOX40 receptor molecule or portions thereof. The association may be non-covalent, wherein the juxtaposition is energetically favored by hydrogen bonding, van der Waals forces, and/or electrostatic interactions, or it may be covalent.

In addition, all or a portion of the structural coordinates provided in any of Tables 8, 9, and/or 10 can be utilized to generate nmr assignments. The X-ray coordinates for OX40L-OX40 can be used to assist in determining NMR structures of OX40L, OX40, or complexes thereof with other entities. Briefly, chemical shift information may be obtained from NMR determined chemical shifts (using HSQC, TOCSY, NOESY and other experiments) or may be calculated from the coordinates of OX40, OX40L, or the OX40-OX40L complex using computer programs such as SHIFTS or other custom programs. Comparison of the chemical shifts of free and bound molecules can be used to map binding sites of protein or small molecule ligands on either OX40, OX40L, or the OX40-OX40L complex. In addition, an expected pattern of through-space proton-proton interactions (NOESY crosspeaks) can be generated from the X-ray coordinated and compared to those measured in an NMR spectra (NOESY) using NMR structure determination programs such as ATNOS, ARIA, CNS, CCPN or other custom programs. This information may then be used to refine the structures of complexes between OX40, OX40L, OX40-OX40L and other entities.

3. Structurally Equivalent Crystal Structures

Various computational analyses can be used to determine whether a molecule or portions of the molecule defining structure features are “structurally equivalent,” defined in terms of its three-dimensional structure, to all or part of a mOX40L, mOX40L-hOX40 receptor complex, or hOX40L-hOX40 receptor complex. Such analyses may be carried out in current software applications, such as the Molecular Similarity application of QUANTA (Molecular Simulations Inc., San Diego, Calif.), Version 4.1, and as described in the accompanying User's Guide.

The Molecular Similarity application permits comparisons between different structures, different conformations of the same structure, and different parts of the same structure. A procedure used in Molecular Similarity to compare structures comprises: 1) loading the structures to be compared; 2) defining the atom equivalences in these structures; 3) performing a fitting operation; and 4) analyzing the results.

One structure is identified as the target (i.e., the fixed structure); all remaining structures are working structures (i.e., moving structures). Since atom equivalency within QUANTA is defined by user input, for the purpose of this disclosure equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in Angstroms, is reported by QUANTA.

Structurally equivalent crystal structures have portions of the two molecules that are substantially identical, within an acceptable margin of error. The margin of error can be calculated by methods known to those of skill in the art. In some embodiments, any molecule or molecular complex or any portion thereof, that has a root mean square deviation of conserved residue backbone atoms (N, Cα, C, O) of less than about 0.70 Å, preferably 0.5 Å. For example, structurally equivalent molecules or molecular complexes are those that are defined by the entire set of structure coordinates listed in Tables 8, 9, and/or 10± a root mean square deviation from the conserved backbone atoms of those amino acids of not more than 0.70 Å, preferably 0.5 Å. The term “root mean square deviation” means the square root of the arithmetic mean of the squares of the deviations. It is a way to express the deviation or variation from a trend or object. For purposes of this disclosure, the “root mean square deviation” defines the variation in the backbone of a protein from the backbone of mOX40L, mOX40L-hOX40 receptor complex, or mOX40L-hOX40 receptor complex (as defined by the structure coordinates of the complex as described herein) or a defining structural feature thereof.

4. Structurally Homologous Molecules, Molecular Complexes, and Crystal Structures

Structure coordinates can be used to aid in obtaining structural information about another crystallized molecule or molecular complex. The method of the disclosure allows determination of at least a portion of the three-dimensional structure of molecules or molecular complexes that contain one or more structural features that are similar to structural features of at least a portion of the mOX40L, mOX40L-hOX40 receptor complex, or hOX40L-hOX40 receptor complex. These molecules are referred to herein as “structurally homologous” to mOX40L, mOX40L-hOX40 receptor complex, or hOX40L-hOX40 receptor complex. Similar structural features can include, for example, regions of amino acid identity, conserved active site or binding site motifs, and similarly arranged secondary structural elements.

Structural elements in OX40L include several domains including A, A″, B', B, C, D, E, F, G, and H β sheet strands that form jelly roll β sandwich monomer sheets as follows: A′AHCF and B′BGDE. The OX40L forms a flower like trimer with interface formed by a layer of generally hydrophobic residues along the C strand, F strand, and C terminal extension (amino acid residues 175-183 in hOX40L and residues 178-191 in mOX40L). The monomers are splayed out and form an angle of about 45° with respect to the trimer axis. The trimer interface is compact with about 2600 Å accessible surface area buried.

In some embodiments, a portion of the three dimensional structure refers to structural domains of the OX40L, including the A, A″, B′, B, C, D, E, F, G, H β sheets or the loops connecting the β sheets including amino acid residues corresponding to Q80, E82, D98, S110, Y119, E123, E124, T144, Y145, K146, D147, D162, H164, V165, F180, L183 of the amino acid sequence of SEQ ID NO:3 or combinations thereof. In some embodiments, the structural domains include A and/or H β sheets, and/or amino acids linking β sheets A″ to B′, B to C, C to D, D to E, F to G, G to H, or combinations thereof. In other embodiments, the structural domains refer to the amino acids found at the interface of the trimer including amino acids corresponding to those found at positions Q175, L102, L138, or mixtures thereof. The trimer interface may comprise amino acid residues 98 to 183 of the amino acid sequence of SEQ ID NO:3, or may comprise amino acid residues 99 to 191 of the amino acid sequence of SEQ ID NO:1. In some embodiments, the structural element is the mOX40L and/or hOX40L binding site for hOX40 receptor.

Structural elements in hOX40 receptor include three full CRDs and a partial C-terminal CRD which form a contiguous structure. CRD1 and CRD2 form a rigid unit while there is some rotational freedom between the subdomains of CRD3 and the partial CRD4. CRD3 in hOX40 differs more than CRD1 and CRD2 from its counterpart in TNFR1 or DR5 as it is smaller and lacks one of the canonical disulfides. CRD3 contains an A1, B1 module rather than the A1, B2 architecture of CRD1 and 2. In the case of OX40, the disulfide formed by the fourth and sixth cysteines in CRD3 (the 4-6 disulfide) is missing. This missing disulfide in hOX40 CRD3 is not replaced by either hydrogen bonds or hydrophobic interactions, but rather the entire module smaller due to shortening and re-arrangement of intervening loops. This re-arrangement causes the backbone of the remaining 3-5 disulfide to be in an anti-parallel orientation rather than the parallel orientation seen in B2 modules. A long disulfide containing loop follows the B1 module. The connectivity and structure of this loop are compatible with it being an A1 module of a vestigial CRD4.

In some embodiments, a portion of the three dimensional structure refers to the domains of hOX40 receptor including CRD1, CRD2, CRD3, CRD4, or combinations thereof. In some embodiments, CRD1 comprises amino acids 29-65; CRD2 comprises amino acids 66-108; CRD3 comprises amino acids 109-147; of the amino acid sequence of SEQ ID NO:2. In some embodiments, CRD1 includes at least residues 31-64 including Cys 31, 42, 43, 56, 46 and 64; CRD2 includes at least residues 67-107 including Cys 67, Cys 81, Cys 84, Cys 99, Cys 87 and Cys 107; CRD3 includes at least 109-141 including cys 109, cys 125, cys 128, and cys 141; and CRD4 includes at least residues 147-166 including cys 147 and cys 166 of the amino acid sequence of SEQ ID NO:2. In some embodiments, the structural domain is that of the CRD1, CRD2 or CDRD3. In some embodiments, the structural feature is the binding site on hOX40 receptor for hOX40L or mOX40L. In some embodiments, structurally homologous molecules do not include the CRD2 domain of DR5, or the CRD3 domain of hGITR, hRANK and/or hEDAR.

Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Two amino acid sequences are compared using the BLAST program, version 2.0.9, of the BLAST 2 search algorithm, as described by Tatusova et al. (56), and available at http:www.ncbi.nlm.nih.gov/BLAST/. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.”

In some embodiments, a structurally homologous molecule is a protein that has an amino acid sequence having at least 80% identity with a wild type or recombinant amino acid sequence of mOX40L, hOX40 receptor, or hOX40L having a sequence of SEQ ID NO:1, SEQ ID NO:2, or SEQ ID NO:3, of fragments thereof as described herein respectively. More preferably, a protein that is structurally homologous to mOX40L, hOX40 receptor, or hOX40L includes at least one contiguous stretch of at least 25, 50, or even up to 100 amino acids that have at least 80% amino acid sequence identity with the analogous portion of the wild type or recombinant mOX40L, hOX40 receptor, or hOX40L. Methods for generating structural information about the structurally homologous molecule or molecular complex are well known and include, for example, molecular replacement techniques.

Therefore, in another embodiment this disclosure provides a method of utilizing molecular replacement to obtain structural information about a molecule or molecular complex whose structure is unknown comprising:

(a) generating an X-ray diffraction pattern from a crystallized molecule or molecular complex of unknown or incompletely known structure; and

(b) applying at least a portion of the structural coordinates of mOX40L, mOX40L-hOX40 receptor complex, or hOX40L-hOX40 receptor complex to the X-ray diffraction pattern to generate a three-dimensional electron density map of the molecule or molecular complex whose structure is unknown or incompletely known.

By using molecular replacement, all or part of the structure coordinates of mOX40L, mOX40L-hOX40 receptor complex, or hOX40L-hOX40 receptor complex as provided by this disclosure can be used to determine the unsolved structure of a crystallized molecule or molecular complex more quickly and efficiently than attempting to determine such information ab initio.

Molecular replacement can provide an accurate estimation of the phases for an unknown or incompletely known structure. Phases are one factor in equations that are used to solve crystal structures, and this factor cannot be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a structurally homologous portion has been solved, molecular replacement using the known structure provide a useful estimate of the phases for the unknown or incompletely known structure.

Thus, this method involves generating a preliminary model of a molecule or molecular complex whose structure coordinates are unknown, by orienting and positioning the relevant portion of the mOX40L, mOX40L-hOX40 receptor complex, or hOX40L-hOX40 receptor complex within the unit cell of the crystal of the unknown molecule or molecular complex. This orientation or positioning is conducted so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure. This map, in turn, can be subjected to established and well-known model building and structure refinement techniques to provide a final, accurate structure of the unknown crystallized molecule or molecular complex (see for example, Lattman, Methods in Enzymology, 115:55-77 (1985)).

Structural information about a portion of any crystallized molecule or molecular complex that is sufficiently structurally homologous to a portion of mOX40L, mOX40L-hOX40 receptor complex, or hOX40L-hOX40 receptor complex can be solved by this method. In addition to a molecule that shares one or more structural features with the OX40L, such as the β sandwich monomer sheets and/or trimer interface, and/or OX40 receptor CRD domains described above, a molecule that has similar bioactivity, such as the same catalytic activity, substrate specificity or ligand binding activity as mOX40L, mOX40L-hOX40 receptor complex, or hOX40L-hOX40 receptor complex, may also be sufficiently structurally homologous to a portion of the mOX40L, mOX40L-hOX40 receptor complex, or hOX40L-hOX40 receptor complex to permit use of the structure coordinates of described herein to solve its crystal structure or identify structural features that are similar to those identified in the mOX40L, mOX40L-hOX40 receptor complex, or hOX40L-hOX40 receptor complex. It will be appreciated that amino acid residues in the structurally homologous molecule identified as corresponding to the OX40L or hOX40 receptor structural feature may have different amino acid numbering.

In one embodiment of the disclosure, the method of molecular replacement is utilized to obtain structural information about a molecule or molecular complex, wherein the molecule or molecular complex is homologous to at least one mOX40L, mOX40L-hOX40 receptor complex, or hOX40L-hOX40 receptor complex structural feature or homolog. In the context of the present disclosure, a “structural homolog” of the mOX40L, mOX40L-hOX40 receptor complex, or hOX40L-hOX40 receptor complex is a protein that contains one or more amino acid substitutions, deletions, additions, or rearrangements with respect to the amino acid sequence of mOX40L, mOX40L-hOX40 receptor complex, or hOX40L-hOX40 receptor complex, but that, when folded into its native conformation, exhibits or is reasonably expected to exhibit at least a portion of the tertiary (three-dimensional) structure of at least a portion of mOX40L, mOX40L-hOX40 receptor complex, or hOX40L-hOX40 receptor complex.

A heavy atom derivative of mOX40L, mOX40L-hOX40 receptor complex, or hOX40L-hOX40 receptor complex is a homolog. The term “heavy atom derivative” refers to derivatives of mOX40L, mOX40L-hOX40 receptor complex, or hOX40L-hOX40 receptor complex by chemically modifying a crystal of mOX40L, mOX40L-hOX40 receptor complex, or hOX40L-hOX40 receptor complex. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the protein (Blundell, et al., 1976, Protein Crystallography, Academic Press, San Diego, Calif.).

An isotopically labeled polypeptide of mOX40L, hOX40 receptor complex, or hOX40L complex is a derivative. Isotopic labels include ¹³C, ¹⁵N, ²H, ³H, ³¹P, ²³Na, ¹⁴N, and/or ¹⁹F.

mOX40L, hOX40 receptor, or hOX40L polypeptides may be prepared, for example, by expression of cDNA previously altered in its coding sequence by oligonucleotide-directed mutagenesis as described herein. Polypeptides may also be generated by site-specific incorporation of unnatural amino acids into mOX40L, hOX40 receptor, or hOX40L polypeptides using known biosynthetic methods (Noren, et al., Science, 244:182-88 (1989)). hOX40L polypeptides with amino acid substitutions have been described herein.

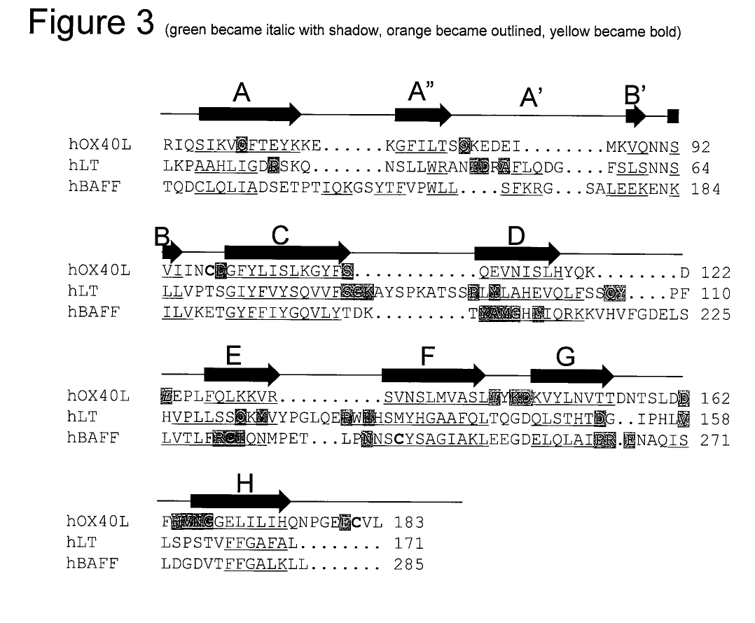

For example, structurally homologous molecules can contain deletions or additions of one or more contiguous or noncontiguous amino acids, such as a loop or a domain. For example, domains and loops of hOX40L are shown in FIG. 3 and described herein. Structurally homologous molecules also include “modified” mOX40L, hOX40 receptor, or hOX40L polypeptides molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and like modifications. It will be appreciated that amino acid residues in the structurally homologous molecule identified as corresponding to mOX40L, hOX40 receptor, or hOX40L polypeptides or other structural feature may have different amino acid numbering.

All of the complexes referred to above may be studied using well-known X-ray diffraction techniques and may be refined versus 1.5-3.5 Å resolution X-ray data to an R-factor of about 0.30 or less using computer software, such as X-PLOR (Yale University, distributed by Molecular Simulations, Inc.) (see, for example, Blundell, et al. 1976. Protein Crystallography, Academic Press, San Diego, Calif., and Methods in Enzymology, Vol. 114 & 115, H. W. Wyckoff et al., eds., Academic Press (1985)). This information may thus be used to optimize known mOX40L, hOX40 receptor, or hOX40L modulators, and more importantly, to design new mOX40L, hOX40 receptor, or hOX40L modulators.

In addition, all or a portion of the structural coordinates may be utilized to make nmr assignments of the residues of all or a portion of mOX40L, hOX40L, and/or hOX40 receptor. The X-ray coordinates for OX40L-OX40 can be used to assist in determining NMR structures of OX40L, OX40, or complexes thereof, with other entities. Briefly, chemical shift information may be obtained from NMR determined chemical shifts (using HSQC, TOCSY, NOESY and other experiments) or may be calculated from the coordinates of OX40, OX40L, or the OX40-OX40L complex using computer programs such as SHIFTS or other custom programs. Comparison of the chemical shifts of free and bound molecules can be used to map binding sites of protein or small molecule ligands on either OX40, OX40L, or the OX40-OX40L complex. In addition, an expected pattern of through-space proton-proton interactions (NOESY crosspeaks) can be generated from the X-ray coordinated and compared to those measured in an NMR spectra (NOESY) using NMR structure determination programs such as ATNOS, ARIA, CNS, CCPN or other custom programs. This information may then be used to refine the structures of complexes between OX40, OX40L, OX40-OX40L and other entities.

The disclosure also includes the unique three-dimensional configuration defined by a set of points defined by the structure coordinates for a molecule or molecular complex structurally homologous to mOX40L, hOX40 receptor, or hOX40L polypeptides as determined using the method of the present disclosure, structurally equivalent configurations, configuration of homologous sequences or structures and magnetic storage media including such sets of structure coordinates.

5. Homology Modeling

Using homology modeling, a computer model of a homolog of a mOX40L, hOX40 receptor, or hOX40L polypeptides or complexes thereof can be built or refined without crystallizing the homolog. First, a preliminary model of the homolog is created by sequence alignment with mOX40L, hOX40 receptor, or hOX40L polypeptides, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. If the homolog has been crystallized, the final homology model can be used to solve the crystal structure of the homolog by molecular replacement, as described above. Next, the preliminary model is subjected to energy minimization to yield an energy-minimized model. The energy-minimized model may contain regions where stereochemistry restraints are violated, in which case such regions are remodeled to obtain a final homology model. The homology model is positioned according to the results of molecular replacement, and subjected to further refinement including molecular dynamics calculations.

6. Methods for Identification of Modulators of mOX40L, hOX40L, and/or OX40 Receptor

Potent and selective ligands that modulate activity (antagonists and agonists) of mOX40L, hOX40 receptor, or hOX40L polypeptides are identified using the three-dimensional model of the binding site on mOX40L for the hOX40 receptor, or on the binding site on the hOX40L for the hOX40 receptor, or the binding site on hOX40 receptor for mOX40L or hOX40L and/or other structural features produced using all or a portion of the coordinates of Tables 8, 9 and/or Table 10. Using these models, ligands that associate with such a binding site with or without a ligand or inhibitor are identified, and the result of the interactions is modeled. In some embodiments, agents identified as candidate molecules for modulating the activity of mOX40L, hOX40L, and/or hOX40 receptor can be screened against known bioassays. For example, the ability of an agent to inhibit the binding of mOX40L and/or hOX40L to hOX40 receptor can be measured using assays known in the art. Elisa may be used to determine whether agent inhibits of binding of OX40 to OX40L, or vice versa. Bioassays for OX40L antagonist: detect inhibition of OX40-induced NFKβ signaling, detect inhibition of T cell proliferation using MLR assay. These assays are described in co-owned co-pending U.S. Ser. No. 60/751,377 (filed Dec. 16, 2005) and WO2006/029879.

Using the modeling information and the assays described, one can identify agents that possess mOX40L, hOX40L, and/or hOX40 receptor modulating properties.

The methods of the disclosure also include methods of identifying molecules that mimic binding of mOX40L and/or hOX40L to hOX40 receptor, but do not activate the receptor, or mimic binding of hOX40 receptor to mOX40L and/or hOX40L, but do not activate the OX40 ligand. These molecules can be identified using the three-dimensional model of mOX40L, hOX40L-hOX40 receptor complexes, and/or mOX40L-OX40 receptor complexes using all or a portion of the coordinates of Tables 8, 9, and/or 10. The methods of the disclosure further comprise testing the test agent in an assay for binding and/or modulating activity.

In another embodiment, a candidate modulator can be identified using a biological assay such as binding to hOX40 receptor or receptor activation, or binding to mOX40L or hOX40L. The candidate modulator can then serve as a model to design similar agents and/or to modify the candidate modulator for example, to improve characteristics such as binding to mOX40L, hOX40L, OX40 receptor or complexes thereof. Design or modification of candidate modulators can be accomplished using the crystal structure coordinates and available software.

Binding Site and Other Structural Features

Applicants' disclosure provides information inter alia about the shape and structure of the binding site of mOX40L for the hOX40 receptor, hOX40L for the hOX40 receptor, and/or OX40 receptor for mOX40L and/or hOX40L. Binding sites are of significant utility in fields such as drug discovery. The association of ligands or substrates with the binding sites of their corresponding receptors or enzymes is the basis of many biological mechanisms of action. Similarly, many drugs exert their biological effects through association with the binding sites of receptors and enzymes. Such associations may occur with all or any part of the binding site. An understanding of such associations helps lead to the design of drugs having more favorable associations with their target, and thus improved biological effects. Therefore, this information is valuable in designing potential modulators of mOX40L, hOX40L, and/or hOX40 receptor binding sites, as discussed in more detail below.

In some embodiments, a hOX40L binding site for hOX40 receptor comprises, consists essentially of, or consists of at least one amino acid residue corresponding to an amino acid residue selected form the group consisting of Q65, T67, Q80, E82, D98, Y108, F109, S110, Y119, E123, E124, S142, T144, Y145, K146, D147, D162, H164, V165, N166, G167, F180, and V182 of the polypeptide comprising SEQ ID NO:3, and combinations thereof. In some embodiments, a hOX40L binding site for hOX40 receptor comprises, consists essentially of, or consists of at least one amino acid residue corresponding to an amino acid residue selected from the group consisting of Q65, T67, E82, D98, 5110, E123, T144, Y145, K146, D147, D162, H164, V165, N166, G167, F180, and V182 of the polypeptide comprising SEQ ID NO:3, and combinations thereof. In some embodiments, a hOX40L binding site for hOX40 receptor comprises, consists essentially of, or consists of at least one amino acid residue corresponding to an amino acid selected from the group consisting of D98, T144, D162, N166, and F180 of the polypeptide comprising SEQ ID NO:3, and combinations thereof. In some embodiments, a hOX40L functional binding site for hOX40 receptor comprises, consists essentially of, or consists of at least one amino acid residue corresponding to an amino acid residue selected from the group consisting of Q80, D162, T144, E123, N166 and F180 of the polypeptide comprising SEQ ID NO:3, and combinations thereof. In some embodiments, a hOX40L binding site for the hOX40 receptor includes 1, 2, 3, 4, 5, 6, 7, 8, 9, 10, 11, 12, 13, 14, 15, 16, 17, 18, 19, 20, 21, 22, 23 or all of the identified amino acids or combinations thereof. The disclosure includes all or a portion of the crystal structure coordinates for at least one amino acid residue of a hOX40L binding site for hOX40 receptor, such as provided in Table 10.

In some embodiments, a mOX40L binding site for hOX40 receptor comprises, consists essentially of, or consists of at least one amino acid residue corresponding to an amino acid residue selected from the group consisting of R64, A66, T68, S78, Y80, K81, N82, E83, D99, F111, Q112, H119, R121, N125, P126, S145, L146, A147, F148, K149, D150, L166, Q167, I168, N169, G171, Y182, P185, G187, 5188, Y189, and H190 of the polypeptide comprising SEQ ID NO:1, and combinations thereof. In some embodiments, a mOX40L binding site for hOX40 receptor comprises, consists essentially of, or consists of at least one amino acid residue corresponding to an amino acid residue selected from the group consisting of A66, T68, Y80, N82, E83, D99, F111, N125, P126, H119, 5145 A147, F148, K149, D150, Q167, I168, N169, G171 Y182, S188, and Y189 of the polypeptide comprising SEQ ID NO:1, and combinations thereof. In some embodiments, a mOX40L binding site for hOX40 receptor comprises, consists essentially of, or consists of at least one amino acid residue corresponding to an amino acid residue selected from the group consisting of A66, Y80, D99, F111, A147, N169, Y182, and S188 of the polypeptide comprising SEQ ID NO:1, and combinations thereof. In some embodiments, a mOX40L binding site for the hOX40 receptor includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or all of the identified amino acids, or combinations thereof. The disclosure includes all or a portion of the crystal structure coordinates for at least one amino acid residue of a mOX40L binding site for hOX40 receptor, such as provided in Tables 8 or 9.

In some embodiments, a hOX40 receptor binding site for hOX40L comprises, consists essentially of, or consists of at least one amino acid residue corresponding to an amino acid selected from the group consisting of T35, Y36, P37, S38, E45, M52, V53, S54, R55, R65, F71, V75, S78, K79, P80, K82, P83, C84, T85, W86, C87, N88, L89, Y119, K120, V123, and D124 of the polypeptide comprising SEQ ID NO:2, and combinations thereof. In some embodiments, a hOX40 receptor binding site for hOX40L comprises, consists essentially of, or consists of at least one amino acid residue corresponding to an amino acid selected from the group consisting of Y36, P37, S38, E45, V53, R55, S78, K79, P83, T85, W86, C87, N88, Y119, and V123 of the polypeptide comprising SEQ ID NO:2, and combinations thereof. In some embodiments, a hOX40 receptor binding site for hOX40L comprises, consists essentially of, or consists of at least one amino acid residue corresponding to an amino acid selected from the group consisting of V53, R55, S78, K79, C87, and N88 of the polypeptide comprising SEQ ID NO:2, and combinations thereof. In some embodiments, a hOX40 receptor binding site for the hOX40L includes 1, 2, 3, 4, 5, 6, 7, 8, 9, 10, 11, 12, 13, 14, 15, 16, 17, 18, 19, 20, 21, 22, 23, 24, 25, 26, 27, or all of the identified amino acids or combinations thereof. The disclosure includes all or a portion of the crystal structure coordinates for at least one amino acid residue of a hOX40 receptor binding site for hOX40L, such as provided in Table 10.

In some embodiments, a hOX40 receptor binding site for mOX40L comprises, consists essentially of, or consists of at least one amino acid residue corresponding to an amino acid selected from the group consisting of L29, H30, C31, G33, T35, Y36, P37, S38, E45, M52, V53, S54, R55, F71, V75, S78, K79, P80, C81, P83, C84, T85, W86, C87, N88, R90, Y119, K120, and V123 of the polypeptide comprising SEQ ID NO:2, and combinations thereof. In some embodiments, a hOX40 receptor binding site for mOX40L comprises, consists essentially of, or consists of at least one amino acid residue corresponding to an amino acid selected from the group consisting of C31, G33, T35, Y36, P37, S38, E45, M52, V53, S54, R55, F71, S78, K79, P80, P83, C84, W86, N88, and Y119 of the polypeptide comprising SEQ ID NO:2, and combinations thereof. In some embodiments, a hOX40 receptor binding site for mOX40L comprises, consists essentially of, or consists of at least one amino acid residue corresponding to an amino acid selected from the group consisting of C31, Y36, P37, M52, R55, and P80 of the polypeptide comprising SEQ ID NO:2, and combinations thereof. In some embodiments, a hOX40 receptor binding site for the mOX40L includes 1, 2, 3, 4, 5, 6, 7, 8, 9, 10, 11, 12, 13, 14, 15, 16, 17, 18, 19, 20, 21, 22, 23, 24, 25, 26, 27, 28, 29, or all of the identified amino acids or combinations thereof. The disclosure includes all or a portion of the crystal structure coordinates for at least one amino acid residue of a hOX40 receptor binding site for mOX40L, such as provided in Table 9.

In some embodiments, a hOX40 receptor binding site for FIV comprises, consists essentially of, or consists of at least one amino acid corresponding to an amino acid selected from the group consisting of R58, S59, N61, H44, V63 of the polypeptide comprising the amino acid sequence of SEQ ID NO:3, and combinations thereof.

Other structural features include the interface of hOX40L or mOX40L trimers. In some embodiments the interface comprises at least one amino acid or combinations thereof, in a position of OX40L corresponding to L102, L138, or Q175 of the polypeptide comprising the amino acid sequence of SEQ ID NO:3. In some embodiments, the trimer interface includes one or more amino acid residues corresponding to amino acids 98 to 182 or 183 of the amino acid sequence of SEQ ID NO:3. In some embodiments, the trimer interface includes one or more amino acid residues corresponding to amino acids 99 to 191 of SEQ. ID NO:1. In other embodiments, the interface comprises at least one amino acid corresponding to an amino acid selected from the group consisting of D98, G99, F100, Y101, L102, I103, S104, L105, K106, G107, Y108, F109, S110, S134, V135, N136, S137, L138, M139, V140, A141, S142, L143, Q175, N176, P177, G178, E179, F180, C181, V182, L183 of the polypeptide comprising the amino acid sequence of SEQ ID NO:3, and combinations thereof. In some embodiments, a OX40L trimer interface includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the identified amino acids, or combinations thereof. The disclosure includes all or a portion of the crystal structure coordinates for at least one amino acid residue of a hOX40L or mOX40L trimer interface.

Rational Drug Design

Computational techniques can be used to screen, identify, select, design ligands, and combinations thereof, capable of associating with mOX40L, hOX40L, and/or hOX40 receptor or structurally homologous molecules. Candidate modulators of mOX40L, hOX40L, and/or hOX40 receptor may be identified using functional assays, such as binding assays, and novel modulators designed based on the structure of the candidate molecules so identified. Knowledge of the structure coordinates for mOX40L, hOX40L, and/or hOX40 receptor or complexes thereof permits, for example, the design, the identification of synthetic compounds, and like processes, and the design, the identification of other molecules and like processes, that have a shape complementary to the conformation of the mOX40L, hOX40L, and/or hOX40 receptor binding sites. The identification or design of modulators can be de novo or based on a known TNF inhibitor.

In particular, computational techniques can be used to identify or design ligands, such as agonists and/or antagonists, that associate with a mOX40L, hOX40L, and/or hOX40 receptor binding site. Antagonists may bind to or interfere with all or a portion of an active site of mOX40L, hOX40L, and/or hOX40 receptor, and can be competitive, non-competitive, or uncompetitive inhibitors. Once identified and screened for biological activity, these agonists, antagonists, and combinations thereof, may be used therapeutically and/or prophylactically, for example, to block mOX40L, hOX40L, and/or hOX40 receptor activity and thus prevent the onset and/or further progression of diseases associated with mOX40L, hOX40L, and/or hOX40 receptor activity. Structure-activity data for analogues of ligands that bind to or interfere with mOX40L, hOX40L, and/or hOX40 receptor binding sites can also be obtained computationally.

In some embodiments, agonists or antagonists can be designed to include components that preserve and/or strengthen the interactions. For example, such antagonists would include components that are able to associate, for example, hydrogen bond with one or more of Q80, D162, E163, and/or N166, or interact with hydrophobic residues T144 and/or F180 of the hOX40L. In some embodiments, the inhibitor would be designed to be more specific for hOX40L rather than mOX40L by minimizing interactions with one or more residues corresponding to A66, Y80, a147, Y182, 5188, and/or F111 of the mOX40L. Such antagonists or agonists may also include components that are able to interact, for example, with hydrophobic residues A66, A188, and/or F111 of the mOX40L.

In some embodiments, for mOX40L or hOX40L, antagonist or agonist molecules are designed or selected that can associate with at least one or all amino acid residues that comprise, consist essentially of, or consist of at least one amino acid residue corresponding to an amino acid residue in the monomer interface, or mixtures thereof. In some embodiments, the trimer interface includes amino acid residues corresponding to amino acids 98 to 182 or 183 of the amino acid sequence of SEQ ID NO:3. In some embodiments, the trimer interface includes amino acid residues corresponding to amino acids 99 to 191 of SEQ. ID NO:1. In some embodiments, the interface comprises at least one amino acid, or combinations thereof, in a position of OX40L corresponding to D98, G99, F100, Y101, L102, I103, S104, L105, K106, G107, Y108, F109, S110, S134, V135, N136, S137, L138, M139, V140, A141, 5142, L143, Q175, N176, P177, G178, E179, F180, C181, V182, or L183 of SEQ ID NO:3. In some embodiments the agonist or antagonist that can bind at the monomer interface can fit into a groove of about 2600 Angstroms.

In other embodiments, another criteria that may be utilized in the design of modulators is whether the modulator can fit into the binding site on hOX40 receptor. In some embodiments, such antagonists or agonists would include components that are able to associate, for example, hydrogen bond with one or more of R55, K79, N88, C87, or combinations thereof, or associate with one or more of Y36, V53, S78, P83, W86, or combinations thereof, of the hOX40 receptor. The volume of the binding site hOX40 receptor for hOX40L is about 2600 cubic angstroms, split about equally between the hOX40L and hOX40 receptor. The volume of the binding site hOX40 receptor for mOX40L is about 2600 cubic angstroms, split about equally between the mOX40L and hOX40 receptor. In some embodiments, the volume of the identified or designed modulator may be 2600 angstroms or less. The volume of the cavity can be determined by using a program like GRASP to calculate the volume of those atoms.

In some embodiments, the portion of the mOX40L, hOX40L, or hOX40 receptor molecule that binds at the hOX40 receptor binding site or the hOX40L binding site can be used in the initial design of other inhibitors or modulators.

For example, the present disclosure also includes a polypeptide comprising, consisting essentially of, or consisting of a portion or fragment of the OX40L. An embodiment of a polypeptide fragment comprises, consists essentially of, or consists of an amino acid residue starting from any one of amino acid residue 51 to amino acid residue 65 and ending at amino acid residue 180 to residue 183 or residues corresponding to those positions in the polypeptide comprising SEQ ID NO:3. An embodiment of a polypeptide fragment comprises, consists essentially of, or consists of an amino acid residue starting from any one of amino acid residue 51 to amino acid residue 66 and ending at amino acid residue 185 to residue 198 or residues corresponding to those positions in the polypeptide comprising SEQ ID NO:1. In some embodiments, the polypeptide portion has the ability to bind to hOX40 receptor. Fragments of the hOX40L or mOX40L that include binding site residues or trimer interface residues may be utilized in the design of peptidomimetics. As discussed previously, it may be desirable in some embodiments to design a modulator having moieties that can hydrogen bond with V53, W86, or C87 of the receptor.

The present disclosure also includes a polypeptide comprising, consisting essentially of, or consisting of a portion or fragment of a hOX40 receptor. Preferably, the fragment comprises one or more of the CRD domains, preferably the CRD1, CRD2, and/or CRD3 domains. An embodiment of a polypeptide fragment comprises a fragment starting at an amino acid residue 29 to amino acid residue 36 and ending at any one of amino acid residues 147 to amino acid residue 170 of SEQ ID NO:2. The fragment preferably retains the ability to bind to OX40L. As discussed previously, it may be desirable in some embodiments to retain amino acids or other moieties that can hydrogen bond with Q80 or N166, or interact with F180 of the hOX40L. In some embodiments, a modulator may be about 2600 angstroms or less.

In addition, the designed modulator molecules may also be modeled with other known members of TNFRSF or TNFSFL in order to identify features that would enhance the specificity of the modulator for mOX40L, hOX40L, or hOX40 receptor. For example, features may be selected that interact with the smaller trimer interface of hOX40L or that interact with CRD1 residues of hOX40 receptor (such as V53, R55, or Y36).

Other lead compounds that may be utilized to design antagonists or agonists of mOX40L, hOX40L, or hOX40 receptor include already identified classes of small molecule that inhibit TNFRSF or TNFSF. These inhibitors include 6,7-dimethyl-3-[(methyl {2-[methyl({1-[3-(trifluoromethyl)phenyl]-1h-indol-3-yl}methyl)amino]ethyl}amino)methyl]-4h-chromen-4-one and 5-(3-morpholin-4-yl-propyl)-2-(3-nitro-phenyl)-4-thioxo-4,5-dihydro-1-thia-3b,5-diaza-cyclopenta[a]pentalen-6-one.

Several databases are available to search and identify compounds that may bind to and/or inhibit mOX40L, hOX40L, or hOX40 receptor. These databases include include ACD (Molecular Designs Limited), NCI (National Cancer Institute), CCDC (Cambridge Crystaleography Data Center) and DOCK (University of California, San Francisco).

Data stored in a machine-readable storage medium that is capable of displaying a graphical three-dimensional representation of the structure of mOX40L, hOX40L, or hOX40 receptor or a structurally homologous molecule or molecular complex, as identified herein, or portions thereof, may thus be advantageously used for drug discovery. The structure coordinates of the ligand are used to generate a three-dimensional image that can be computationally fit to the three-dimensional image of mOX40L, hOX40L, or hOX40 receptor or a structurally homologous molecule. The three-dimensional molecular structure encoded by the data in the data storage medium can then be computationally evaluated for its ability to associate with ligands. When the molecular structures encoded by the data is displayed in a graphical three-dimensional representation on a computer screen, the protein structure can also be visually inspected for potential association with ligands. The methods of the disclosure further comprise testing the test agent in an assay for binding and/or modulating activity.

One embodiment of the method of drug design involves evaluating the potential association of a candidate ligand with mOX40L, hOX40L, or hOX40 receptor or a structurally homologous molecule or homologous complex, particularly with at least one amino acid residue in a binding site the mOX40L, hOX40L, or hOX40 receptor or a portion of the binding site. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the selected ligand and a ligand binding site or a subsite of the ligand binding site of the molecule or molecular complex; and (b) analyzing the results of the fitting operation to quantify the association between the ligand and the ligand binding site. Optionally, the method further comprises analyzing the ability of the selected ligand to interact with amino acids in the mOX40L, hOX40L, or hOX40 receptor binding site and/or subsite. The method may also further comprise optimizing the fit of the ligand for the binding site of mOX40L, hOX40L, or hOX40 receptor as compared to other TNFRSF or TNFSFL members. Optionally, the selected ligand can be synthesized, cocrystallized with mOX40L, hOX40L, or hOX40 receptor, and further modifications to selected ligand can be made to enhance inhibitory activity or fit in the binding pocket. Other structural features of the mOX40L, hOX40L, or hOX40 receptor such as the monomer interface can also be analyzed in the same manner. The methods of the disclosure further comprise testing the test agentor ligand in an assay for binding and/or modulating activity.

In another embodiment, the method of drug design involves computer-assisted design of ligand that associates with mOX40L, hOX40L, or hOX40 receptor, its homologs, or portions thereof. Ligands can be designed in a step-wise fashion, one fragment at a time, or may be designed as a whole or de novo. Ligands can be designed based on the structure of molecules that can modulate at least one biological function of mOX40L, hOX40L, or hOX40 receptor. In addition, the inhibitors can be modeled on other known inhibitors of TNFRSF or TNFSFL.

In some embodiments, to be a viable drug candidate, the ligand identified or designed according to the method must be capable of structurally associating with at least part of a mOX40L, hOX40L, or hOX40 receptor binding site, and must be able, sterically and energetically, to assume a conformation that allows it to associate with the mOX40L, hOX40L, or hOX40 receptor binding site. Non-covalent molecular interactions important in this association include hydrogen bonding, van der Waals interactions, hydrophobic interactions, and/or electrostatic interactions. Conformational considerations include the overall three-dimensional structure and orientation of the ligand in relation to the ligand binding site, and the spacing between various functional groups of a ligand that directly interact with the mOX40L, hOX40L, or hOX40 receptor binding site or homologs thereof.

Optionally, the potential binding of a ligand to a mOX40L, hOX40L, or hOX40 receptor binding site is analyzed using computer modeling techniques prior to the actual synthesis and testing of the ligand. If these computational experiments suggest insufficient interaction and association between it and the mOX40L, hOX40L, or hOX40 receptor binding site, testing of the ligand is obviated. However, if computer modeling indicates a strong interaction, the molecule may then be synthesized and tested for its ability to bind to or interfere with a mOX40L, hOX40L, or hOX40 receptor binding site. Assays to determine if a compound actually modulates mOX40L, hOX40L, or hOX40 receptor activity can also be performed and are well known in the art.

Several methods can be used to screen ligands or fragments for the ability to associate with a mOX40L, hOX40L, or hOX40 receptor binding site. This process may begin by visual inspection of, for example, a mOX40L, hOX40L, or hOX40 receptor binding site on the computer screen based on the mOX40L, hOX40L, or hOX40 receptor or complexes thereof structure coordinates or other coordinates which define a similar shape generated from the machine-readable storage medium. Selected ligands may then be positioned in a variety of orientations, or docked, within the binding site. Docking may be accomplished using software such as QUANTA and SYBYL, followed by energy minimization and molecular dynamics with standard molecular mechanics force fields, such as CHARMM and AMBER.

Specialized computer programs may also assist in the process of selecting ligands. Examples include GRID (Hubbard, Nature Struct. Biol., 6:711-4 (1999)); MCSS (Miranker et al., Proteins, 11:29-34 (1991)) available from Molecular Simulations, San Diego, Calif.; AUTODOCK (Goodsell et al., Proteins, 8:195-202 (1990)) available from Scripps Research Institute, La Jolla, Calif.; and DOCK (Kuntz et al., J. Mol. Biol., 161:269-88 (1982)) available from University of California, San Francisco, Calif.; Glide (Halgren et al. J. Med. Clin. 47:1750; Flexx, J. Mol. Biol., 261:470 (1996)) and KM (Abagyan et al., J. Mol. Biol., 235:983 (1999)).

There are many ligand design methods including, without limitation, LUDI (Bohm, J. Comput. Aided Mol. Design, 6:61-78 (1992)) available from Molecular Simulations Inc., San Diego, Calif.; LEGEND (Nishibata et al., J. Med. Chem., 36:2921-8 (1993)) available from Molecular Simulations Inc., San Diego, Calif.; LeapFrog, available from Tripos Associates, St. Louis, Mo.; and SPROUT (Gillet et al., J. Comput. Aided Mol. Design, 7:127-53 (1993)) available from the University of Leeds, UK.

Useful programs to aid in searching databases to select ligands include, but are not limited to, CAVEAT (In Molecular Recognition in Chemical and Biological Problems, Royal Chem. Soc. 78:82-196 (1989), 3D Database Systems search as MACCS-3D (J. Med. Clin., 35:2145 (1992), HOOK (available from Molecular Simulateous, Burlington, Mass.) and CLIX (Lawrence et al. Proteins, 12:3141 (1992)).

Databases include ACD (Molecular Designs Limited), NCI (National Cancer Institute), CCDC (Cambridge Crystaleography Data Center) and DOCK (University of California, San Francisco).

Once a compound has been designed or selected by the above methods, the efficiency with which that ligand may bind to or interfere with a mOX40L, hOX40L, or hOX40 receptor binding site may be tested and optimized by computational evaluation. For example, an effective binding site ligand should preferably demonstrate a relatively small difference in energy between its bound and free states (i.e., a small deformation energy of binding). mOX40L, hOX40L, or hOX40 receptor binding site ligands may interact with the binding site in more than one conformation that is similar in overall binding energy. In those cases, the deformation energy of binding is taken to be the difference between the free energy of the ligand and the average energy of the conformations observed when the ligand binds to the protein. A mOX40L, hOX40L, or hOX40 receptor binding site ligand can also preferably be designed with an IC50 0.1 to about 100 nM, more preferably about 10 to 100 nM, more preferably about 50 to 100 nM.

A ligand designed or selected as binding to or interfering with a mOX40L, hOX40L, or hOX40 receptor binding site may be further computationally optimized so that in its bound state it would preferably lack repulsive electrostatic interaction with the receptor or its ligand and with the surrounding water molecules. Such non-complementary electrostatic interactions include repulsive charge-charge, dipole-dipole, and charge-dipole interactions.

Specific computer software is available to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco,); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs can be implemented, for instance, using a Silicon Graphics workstation, such as an Indigo2 with IMPACT graphics. Other hardware systems and software packages will be known to those skilled in the art.

Another approach encompassed by this disclosure is the computational screening of small molecule databases for ligands or compounds that can bind in whole, or in part, to a mOX40L, hOX40L, or hOX40 receptor. In this screening, the quality of fit of such ligands to the binding site may be judged either by shape complementarity or by estimated interaction energy (Meng et al. J. Comp. Chem., 13:505-24 (1992)). In addition, these small molecule databases can be screened for the ability to interact with the amino acids in the mOX40L, hOX40L, or hOX40 receptor binding site as identified herein.

A compound that is identified or designed as a result of any of these methods can be obtained (or synthesized) and tested for its biological activity, for example, binding and/or inhibition of mOX40L, hOX40L, or hOX40 receptor activity. Any high throughput assay may be utilized, including ELISA, competition assays, array based assays.

A method comprises applying at least a portion of the crystallography coordinates of Tables 8, 9, and/or 10 to a computer algorithm that generates a three-dimensional model of mOX40L, hOX40L, or hOX40 receptor suitable for designing molecules that are antagonists or agonists and searching a molecular structure database to identify potential antagonists or agonists. In some embodiments, a portion of the structural coordinates of Tables 8, 9, and/or 10 that define a structural feature, for example, all or a portion of a binding site for an inhibitor on mOX40L, hOX40L, or hOX40 receptor may be utilized. The method may further comprise synthesizing or obtaining the agonist or antagonist and contacting the agonist or antagonist with the mOX40L, hOX40L, or hOX40 receptor and selecting the antagonist or agonist that modulates the mOX40L, hOX40L, or hOX40 receptor activity compared to a control without the agonist or antagonists and/or selecting the antagonist or agonist that binds to the mOX40L, hOX40L, or hOX40 receptor.

7. Machine-Readable Storage Media

Transformation of the structure coordinates for all or a portion of mOX40L, hOX40L, or hOX40 receptor, or one of its ligand binding sites, or structurally homologous molecules as defined below, or for the structural equivalents of any of these molecules or molecular complexes as defined above, into three-dimensional graphical representations of the molecule or complex can be conveniently achieved through the use of commercially-available software.

The disclosure thus further provides a machine-readable storage medium including a data storage material encoded with machine-readable data wherein a machine programmed with instructions for using said data displays an amino acid sequence, a nucleotide sequence and/or a graphical three-dimensional representation of any of the molecule or molecular complexes of this disclosure that have been described above. In a preferred embodiment, the machine-readable data storage medium includes a data storage material encoded with machine-readable data wherein a machine programmed with instructions for using the abovementioned data displays a graphical three-dimensional representation of a molecule or molecular complex including all or any parts of an mOX40L, hOX40L, or hOX40 receptor. In another preferred embodiment, the machine-readable data storage medium includes a data storage material encoded with machine readable data wherein a machine programmed with instructions for using the data displays a graphical three-dimensional representation of a molecule or molecular complex±a root mean square deviation from the atoms of the amino acids of not more than 0.05 Å.

In an alternative embodiment, the machine-readable data storage medium includes a data storage material encoded with a first set of machine readable data which includes the Fourier transform of structure coordinates, and wherein a machine programmed with instructions for using the data is combined with a second set of machine readable data including the X-ray diffraction pattern of a molecule or molecular complex to determine at least a portion of the structure coordinates corresponding to the second set of machine readable data.

Another aspect of the disclosure provides systems, particularly computer based systems, which contain sequence, structure, and/or diffraction data described using such systems are designed to do structure determination of OX40L and/or OX40 receptors or at least one structural feature thereof. A skilled artisan can access this information to model or design a related molecule, structural feature, mimetic or ligand thereof using available software as described herein.

For example, a system for reading a data storage medium may include a computer based system including a central processing unit (“CPU”), a working memory which may be, for example, RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, track balls, touch pads, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this disclosure may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this disclosure using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this disclosure. Such programs are discussed in reference to the computational methods of drug discovery as described herein. References to components of the hardware system are included as appropriate throughout the following description of the data storage medium.

Machine-readable storage devices useful in the present disclosure include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

8. Therapeutic Use

mOX40L, hOX40L, or hOX40 receptor modulator compounds obtained by methods of the invention are useful in a variety of therapeutic settings. For example, mOX40L, hOX40L, or hOX40 receptor antagonists designed or identified using the crystal structure of mOX40L, hOX40L, or hOX40 receptor complexes can be used to treat disorders or conditions, where inhibition or prevention of mOX40L, hOX40L, or hOX40 receptor binding or activity is indicated. Such conditions include conditions associated with TH1 and/or TH2 cells including autoimmune diseases and allergic disorders.

Likewise, mOX40L, hOX40L, or hOX40 receptor agonists designed or identified using the crystal structure of the mOX40L, hOX40L, or hOX40 receptor complex can be used to treat disorders or conditions, where induction or stimulation of mOX40L, hOX40L, or hOX40 receptor is indicated, for example, in the treatment of cancer or tumors.

In one aspect, the disclosure provides use of a modulator in the preparation of a medicament for the therapeutic and/or prophylactic treatment of a disorder, such as an immune disorder. In some embodiments the modulator is an antagonist of OX40L and/or hOX40 receptor. In some embodiments, the disorder is an autoimmune disorder. In some embodiments, the disorder is asthma, atopic dermatitis, allergic rhinitis, inflammatory bowel disease, multiple sclerosis, and/or systemic lupus erythematosus. In some embodiments, the disorder is a disease associated with virus, bacteria or other infectious agent. See US 2005/0069548 A1. Disorder can be graft-verses-host disease or transplant rejection.

In some embodiments, the disorder is arthritis (acute and chronic, rheumatoid arthritis including juvenile-onset rheumatoid arthritis and stages such as rheumatoid synovitis, gout or gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), autoimmune lymphoproliferative disease, inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, hives, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, gastrointestinal inflammation, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, graft-versus-host disease, angioedema such as hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN(RPGN), proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, food allergies, drug allergies, insect allergies, rare allergic disorders such as mastocytosis, allergic reaction, eczema including allergic or atopic eczema, asteatotic eczema, dyshidrotic eczema, and vesicular palmoplantar eczema, asthma such as asthma bronchiale, bronchial asthma, auto-immune asthma allergic asthma, and pediatric asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, SLE, such as cutaneous SLE or subacute cutaneous SLE, neonatal lupus syndrome (NLE), and lupus erythematosus disseminatus, juvenile onset (Type I) diabetes mellitus, including pediatric IDDM, adult onset diabetes mellitus (Type II diabetes), autoimmune diabetes, idiopathic diabetes insipidus, diabetic retinopathy, diabetic nephropathy, diabetic colitis, diabetic large-artery disorder, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia(s), cytopenias such as pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, motoneuritis, allergic neuritis, Behçet's disease/syndrome, Castleman's syndrome, Goodpasture's syndrome, Reynaud's syndrome, Sjögren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury due to an autoimmune condition, preeclampsia, an immune complex disorder such as immune complex nephritis, antibody-mediated nephritis, neuroinflammatory disorders, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia including, for example, idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes, for example, type I (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant-cell hepatitis, chronic active hepatitis or autoimmune chronic active hepatitis, pneumonitis such as lymphoid interstitial pneumonitis (LIP), bronchiolitis obliterans (non-transplant) vs NSIP, Guillain-Barré syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia such as mixed cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, fibrosing mediastinitis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis (systemic inflammatory response syndrome (SIRS)), endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, conjunctivitis, such as vernal catarrh, keratoconjunctivitis sicca, and epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders (cerebral vascular insufficiency) such as arteriosclerotic encephalopathy and arteriosclerotic retinopathy, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, lymphofollicular thymitis, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndromes, including polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, allergic sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, spondyloarthropathies, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism such as chronic arthrorheumatism, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, and endometriosis. Other examples, which in some cases encompass those listed above, include but are not limited to autoimmune rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

For example, Multiple Sclerosis (MS) is a disorder of the central nervous system that affects the brain and spinal cord. Common signs and symptoms of MS include paresthesias in one or more extremities, in the trunk, or on one side of the face; weakness or clumsiness of a leg or hand; or visual disturbances (such as partial blindness and pain in one eye), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances (Berkow et al. (ed.), 1999, Merck Manual of Diagnosis and Therapy: 17th Ed). Current treatments for MS include corticosteroids, beta interferons (Betaferon, Avonex, Rebif), glatiramer acetate (Copaxone), methotrexate, azathioprine, cyclophosphamide, cladribine, baclofen, tizanidine, amitriptyline, carbamazepine (Berkow et al. (ed.), 1999, supra).

Rheumatoid arthritis (RA) is a chronic autoimmune disorder characterized by synovitis of joints that typically affects small and large joints, leading to their progressive destruction (Berkow et al. (ed.), 1999, supra). Symptoms of RA can include stiffness, tenderness, synovial thickening, flexion contractures, visceral nodules, vasculitis causing leg ulcers or mononeuritis multiplex, pleural or pericardial effusions, and fever (Berkow et al. (ed.), 1999, supra).

Current treatments for RA include non-steroidal anti-inflammatory drugs (including salicylates), gold compounds, methotrexate, hydroxychloroquine, sulfasalazine, penicillamine, corticosteroids, and cytotoxic or immunosuppressive drugs. (Berkow et al. (ed.), 1999, Merck Manual of Diagnosis and Therapy: 17th Ed.).

In one aspect, the invention provides use of an agonist of OX40L and/or receptor in the preparation of a medicament for the therapeutic and/or prophylactic treatment of a disorder in which enhancement of immune function is beneficial, such as cancer. Immune function is enhanced by activating OX40 (in presence or absence of antigen-specific immune stimulation), including enhanced anti-tumor immunity. Increasing the immune response by providing molecules which engage the OX-40 receptor, e.g. during T-cell priming, can markedly increase the resistance of an animal to disease, by boosting T-cell recognition of antigens presented by infectious agents, such as bacteria and viruses, as well as tumor cells. Accordingly, the present disclosure provides among other things the use of an OX-40 receptor binding agent as designed or identified herein, or of a nucleic acid encoding an OX-40 receptor binding agent (if the agent is a polypeptide or can be incorporated into a polypeptide), in the manufacture of a pharmaceutical composition for enhancing immune response against an antigen in a mammal, which is either a tumour antigen, or an antigen for which the composition is administered so as to present the OX-40 receptor binding agent to T-cells of the mammal during or shortly after priming of the T-cells by the antigen.

According to a further aspect of the invention a nucleic acid which encodes an OX-40 receptor binding agent identified as described herein that is localised on the surface of a cell, along with tumor cells from a mammal, can be used in the manufacture of a pharmaceutical composition for stimulating the immune response of a mammal to a tumor in the mammal by (a) removing tumor cells from the mammal; (b) attenuating the removed tumor cells; (c) introducing the nucleic acid into the attenuated tumor cells; and (d) administering the thus-treated attenuated tumor cells containing the nucleic acid molecule to the mammal. The OX-40 receptor binding agent in this aspect can be OX-40L or a hOX40 receptor binding fragment thereof, as well as other molecules designed or identified by the methods described herein. The tumor cells can be attenuated prior to or after introducing the nucleic acid molecule.

In an alternative manner of carrying out the invention, a nucleic acid which encodes an OX-40 receptor binding agent as designed or identified herein (if the agent is a polypeptide or can be incorporated into a polypeptide) that is localised on the surface of a cell can be used, along with T-cells from a mammal, in the manufacture of a pharmaceutical composition for enhancing the immune response of a mammal to an antigen, by removing T-cells from the mammal, incubating the removed T-cells ex vivo with an OX-40 receptor binding agent, and returning the thus-treated T-cells to the mammal. Again, the mammal may have a tumor, and the antigen can be a tumor antigen.

More generally, an OX-40 receptor binding agent or a nucleic acid encoding an OX-40 receptor binding agent as identified or designed herein can be used in the manufacture of a pharmaceutical for enhancing immune response against a tumor in a mammal by increasing the amount of OX-40 receptor binding agent at the tumor site. All types of tumor are potentially amenable to treatment by this approach including, for example, carcinoma of the breast, lung, pancreas, ovary, kidney, colon and bladder, as well as melanomas and sarcomas. Nucleic acid molecules encoding a OX-40 receptor binding agent (if the agent is a polypeptide or can be incorporated into a polypeptide) are incorporated into a vector suitable for expression of the OX-40 receptor binding agent in tumor cells. Suitable vectors include plasmid, cosmid and viral vectors, such as retroviruses, adenoviruses and herpesviruses. Because of the high efficiency with which viral vectors infect mammalian cells, viral vectors are expected to offer advantages over other vector types. In addition to a nucleic acid molecule encoding an OX-40 receptor binding agent, other nucleic acid molecules may also be introduced into the vector to further enhance the immunogenic effect. By way of example, such other nucleic acid molecules include nucleic acids encoding MHC class II proteins (including .alpha. and .beta. subunits), and other co-stimulatory molecules, such as B7.1 and B7.2. If desired, a nucleic acid molecule encoding a selectable marker may also be introduced into the vector, such that those tumor cells successfully transformed with the vector can be readily selected.

All publications, patents, and patent documents are incorporated by reference herein, as though individually incorporated by reference. The disclosure has been described with reference to various specific and preferred embodiments and techniques. However, it should be understood that many variations and modifications can be made while remaining within the spirit and scope of the disclosure.

### Example 1

Analysis of the crystal structure of mOX40L and cocrystals of hOX40L with mOX40 and hOX40L was conducted in order to determine how these members of the TNF family interact with one another. The crystal structure provides information useful in the design of potential modulators.

### Materials and Methods:

### Protein Expression:

DNA encoding residues 51-198, 51-183, and 29-170 of murine OX40L (mOX40L), human OX40L (hOX40L), and human OX40 (hOX40) respectively were cloned into a pET 15b expression vector by PCR then subcloned into the baculovirus transfer vector pAcGP67-B (BD Pharmingen) and used for transfection and subsequent viral amplification. To decrease heterogeneity of hOX40L due to glycosylation, residues 90 and 114 were mutated to aspartic acid by site directed mutagenesis (QuickChange mutagenesis kit, Stratagene). Asn to Asp substitutions at positions 152 and 157 severely decreased expression levels and were therefore not pursued.

The resulting viral stocks were used for protein expression in High Five cells (available from Invitrogen) Following growth at 27° C. for 3 days, cell culture media was harvested by centrifugation, treated with nickel chloride, calcium chloride, and Tris buffer, pH 8.0, then filtered prior to passage over a Ni-NTA column to isolate recombinant proteins. His-Tags were removed by thrombin cleavage overnight and the proteins were subsequently further purified by size exclusion chromatography on an S-75 or S-200 column prior to concentration. An N-terminal amino acid sequence of GSHM that was a part of the viral vector remained present on the polypeptides. Complexes of mOX40L-hOX40 and hOX40L-hOX40 were further purified by size exclusion chromatography. Final buffer conditions were 100 mM NaCl, 20 mM Tris, pH 8.0 for mOX40, and 100 mM NaCl, 20 mM Tris, pH 8.2 for mOX40-OX40 and hOX40-OX40. All proteins sequences were verified by mass spectrometry and N-terminal sequencing.

### Crystallization and Data Collection:

Crystals of mOX40L grew by vapor diffusion after approximately 2 weeks in sitting drops containing 1 uL protein and 1 ul well solution consisting of 0.1 M NaAcetate pH 4.5, 2.0 M Ammonium Sulfate at 19° C. Crystals of mOX40L-hOX40 and hOX40L-hOX40 were grown by the same procedure but with well solution of 0.1 M NaCl, 0.1 M Bis Tris pH 6.5, 1.4 M Ammonium Sulfate and 8% PEG 20,000, 0.1 M MES pH 6.5, respectively. Prior to data collections, mOX40L, mOX40L-hOX40, and hOX40L-hOX40 were immersed in artificial mother liquor consisting of the well solution with water replaced by either 20% glycerol (mOX40L) or 20-25% ethylene glycol (mOX40L-hOX40, hOX40L-hOX40). For phasing, mOX40L crystals were soaked for 60 s in cryo-protectant solution supplemented with 1.2 M NaBr prior to flash-cooling in liquid nitrogen. A four wavelength Br MAD experiment was collected at beam line 5.0.2 at ALS. Subsequently a 1.45 Å native data set was collected at beamline F1 at CHESS (Cornell High Energy Synchroton Source). Heavy atom location and phase refinement of the mOX40L structure was performed using the program SHARP. (Statistical Heavy Atom Refinement and Phasing available at Center for Structural Biology Core website at Yale University.) Native data sets for mOX40L-hOX40 and hOX40L-hOX40 were collected at beamlines 19ID at ADP (Advanced Photon Source Argonne National Laboratory) and by MXpress at ESRF (European Synchroton Radiation Facility). All data sets were processed using the HKL package (Otwinowsk and Minor, 1997).

The structures of mOX40L-hOX40 and hOX40L-hOX40 were solved by molecular replacement with the program AMoRe (Navaza, 2001) using the refined mOX40L and mOX40L-OX40 structures respectively as search models. Refinement of all structures was done using the program REFMAC (Winn et al., 2003). See Table 1 for crystallographic statistics.

### Results

### Structure of OX40L

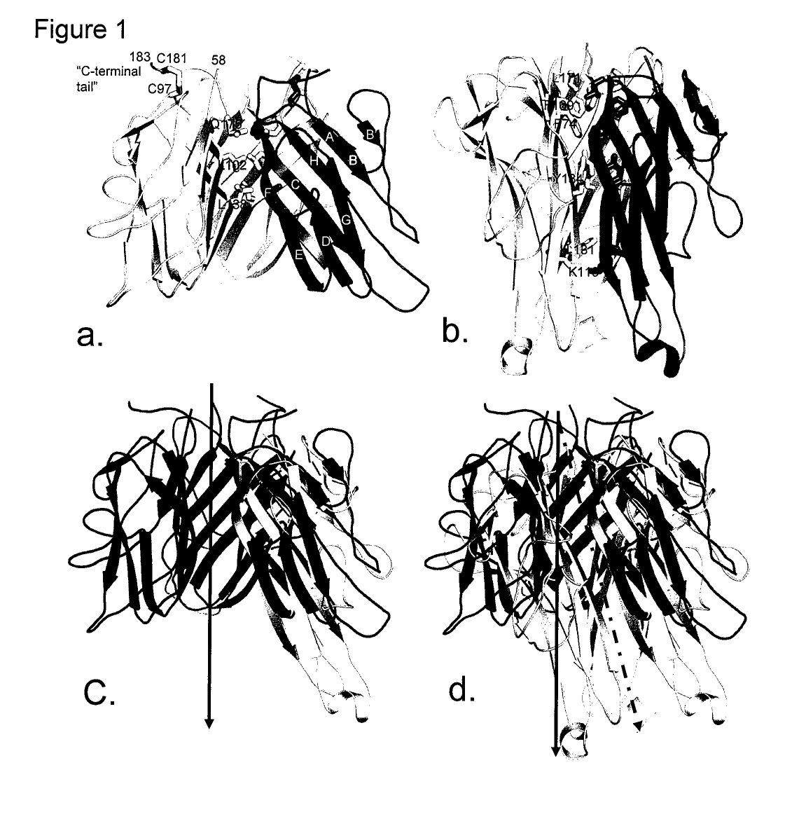

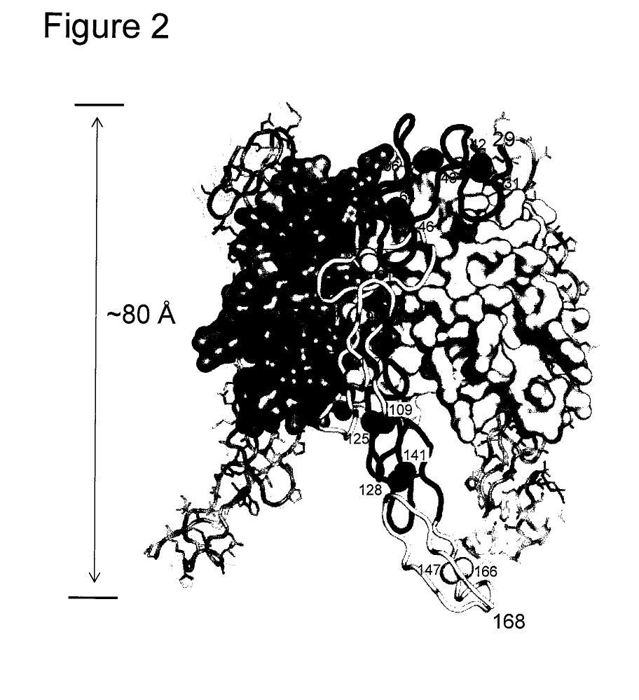

The structure of mOX40L was solved using MAD phasing with NaBr soaked crystals (Table 1). This approach was used after attempts at using molecular replacement to solve either the mOX40L or receptor complexes failed due to the low sequence homology and structural similarity between OX40L and available structures of members of the TNFSF. The refined 1.45 Å mOX40L structure was then used to solve the structures of the hOX40-mOX40L and hOX40-hOX40L complexes by molecular replacement (Table 1). These structures show that the OX40L protomer is brick shaped and packs together to form flower-like trimers. These trimers lack the pyramidal shape typical of more conventional TNFSF members such as Apo2L/TRAIL, TNF, or LT (FIG. 1). However, despite the differences in the appearance and assembly of the ligand, hOX40 binds at the monomer-monomer interface as seen in other structures of multi-domain TNFRSF-ligand complexes (Bodmer et al., Trends Biochem Sci 27:19-26 (2002)) (FIG. 2).

Human and murine OX40L are less similar to each other than many other TNFSF orthologs with only ˜40% sequence identity, yet they share the same distinctive features. Both murine and human OX40L are very compact making OX40L more representative of smaller members of the TNFSF such as GITRL and CD27L. The OX40L TNF homology domain is followed by a C-terminal extension including residues 175-183 in hOX40L and residues 178-191 in mOX40L (FIG. 1). In hOX40L the entire extension is ordered and interacts with the BC loop both by backbone-hydrogen bonds as well as via a conserved disulfide between residues 181 and 97. A second disulfide in murine OX40L contacts the AA′ loop to the GH loop and is not conserved in hOX40L. In both human and murine OX40L, the strands and loops comprising the jelly-roll B-sandwich monomer sheets (A′AHCF and B′BGDE) are significantly shorter than in other TNFSF members such as TNF or Apo2L/TRAIL. In addition to the compact TNF homology domain, hOX40L has a six residue connection to the N-terminal transmembrane domain. These residues are expected to form a short unstructured linker and are not visible in the electron density.

The trimeric packing of TNFSF members is generally very well conserved even among the more distant family members such as BAFF and EDA. Unexpectedly, the structures of human and murine OX40L show that the protomers assemble differently into a trimer than in other structurally characterized members of the TNFSF (FIG. 1). In OX40L, the monomers are splayed out and form an angle of ˜45° with respect to the trimer axis which differs from other TNFL by ˜15° rotation of the monomer. For most TNFSF members, as exemplified by LT, the angle between the AHCF sheet and the trimer axis is 25-30°. This difference in trimer assembly correlates with the lack of sequence conservation between OX40L and other members of the TNFSF (FIG. 3) and accounts for the difficulty in generating high quality homology models of the OX40L trimer.

Another unusual aspect of the OX40L trimer is that human OX40L and murine OX40L have much smaller trimer interfaces than other structurally characterized TNF ligands (FIG. 1). The hOX40L trimer interface is particularly compact with only ˜2600 Å² of accessible surface area buried upon trimer formation in comparison to 12,000 Å² or 5-fold more buried by BAFF and LT (BAFF, LT both ˜12000 Å; hOX40L ˜2600 Å; mOX40L, ˜4000 Å). An additional striking difference between the OX40L and other TNFSF is the absence in OX40L of the characteristic “tiles” of alternating aromatic or hydrophobic residues along the trimer axis first seen in the structure of TNF (Eck and Sprang, 1989; Jones et al., 1992). Instead, the trimer interface of murine and human OX40L is formed by a very short layer of generally hydrophobic residues from the C-strand (L102), F-strand (L138), and the C-terminal tail (Q175). In murine OX40L, the corresponding residues are 1103, T141 and V175. In addition, L178 from the murine OX40L C-terminal tail also contributes to the trimer interface.

Structure of hOX40 Receptor

In contrast to the distinctive structure of OX40L, hOX40 is a relatively conventional multi-domain TNFR. It is composed of three full CRDs and a partial C-terminal CRD which form a contiguous structure. Superposition of the three independent copies of hOX40 (two in the mOX40L-hOX40 asymmetric unit and one in the hOX40L-hOX40 asymmetric unit) reveals that CRD1 and CRD2 form a rigid unit while there is some rotational freedom between the subdomains of CRD3 and the partial CRD4. The first and second CRD have the same disulfide connectivity as the corresponding domains of TNFR1, and in the terminology of Naismith et al are composed of A1-B2 modules (Naismith and Sprang, cited supra). As in other TNFR such as DR5, CRD2 is the best conserved structurally with only a 1 residue deletion with respect to TNFR1 and rmsd of 0.9 Å² on all equivalent C-α when superimposed on TNFR1CRD2 (residues 55-97).

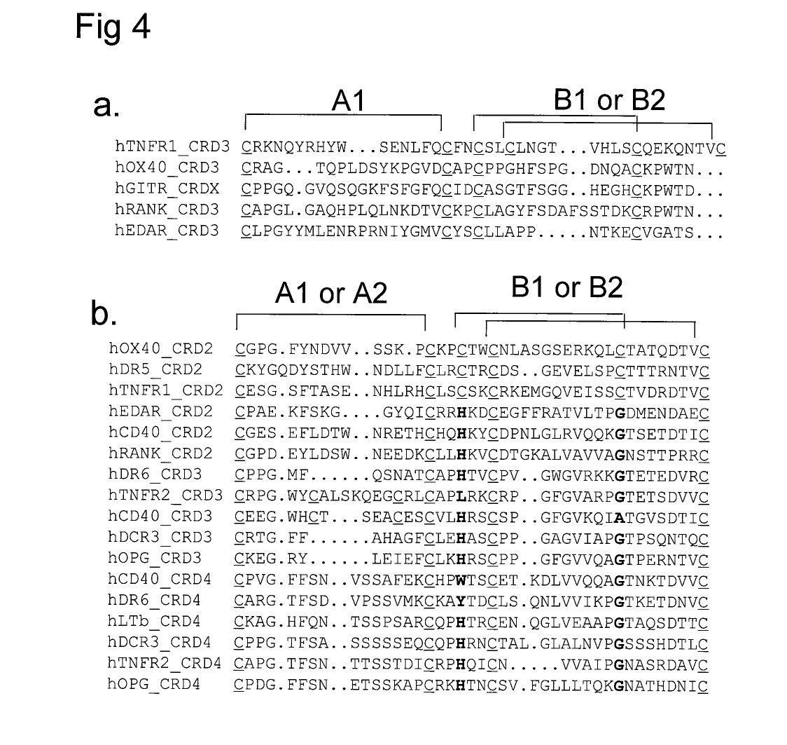

CRD3 in hOX40 differs more than CRD1 and CRD2 from its counterpart in TNFR1 or DR5 as it is smaller and lacks one of the canonical disulfides (FIG. 4). CRD3 contains an A1, B1 module rather than the A1, B2 architecture of CRD1 and 2. This structure of hOX40 CRD3 contains the first experimentally determined B1 subdomain. As discussed above, B1 modules are defined in part by their lack of a conserved disulfide (Naismith and Sprang, cited supra). In the case of hOX40, the disulfide formed by the fourth and sixth cysteines in CRD3 (the 4-6 disulfide) is missing. This missing disulfide in hOX40 CRD3 is not replaced by either hydrogen bonds or hydrophobic interactions but rather the entire module is smaller due to shortening and re-arrangement of intervening loops. This re-arrangement causes the backbone of the remaining 3-5 disulfide to be in an anti-parallel orientation rather than the parallel orientation seen in B2 modules. A long disulfide containing loop follows the B1 module. The connectivity and structure of this loop are compatible with it being an A1 module of a vestigial CRD4.

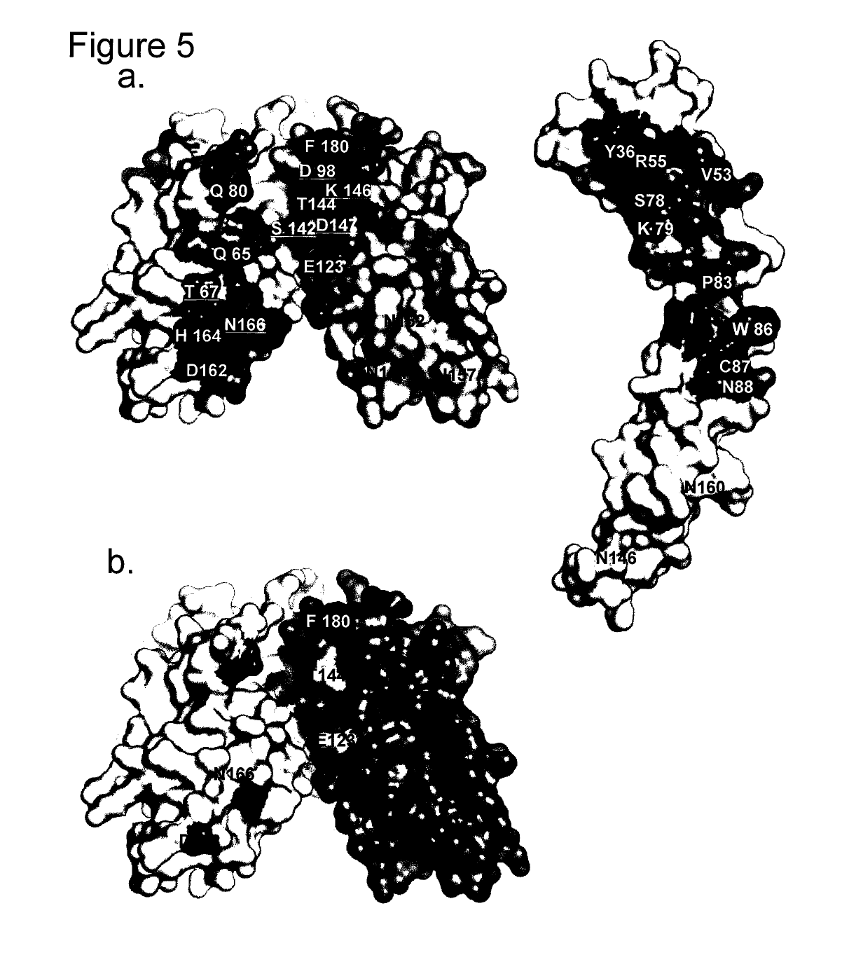

B1 modules have been detected in the sequences of a number of other TNFRSF including OPG, RANK, TNFR2, LTβ-R, HVEM, DcR3, CD30, 4-1BB, GITR, EDAR and RELT. Examining the sequences of these B1 modules along with the structure of the OX40 B1 modules indicates that the B1 modules form two distinct groups based on their size and on which disulfide pair is lacking: a smaller groups consisting of OX40-like B1 modules which lack the 4-6 disulfide and a larger groups of B-1 modules that are more B2-like and lack the 3-5 disulfide (FIG. 5). Like OX40, GITR, EDAR (CRD3) and possibly RANK (CRD3) all possess the smaller B1 module lacking the 4-6 disulfide. The second, more B2-like group is defined by having similar sequence and cysteine spacing as B2-modules. In this group, the 3-5 disulfide is replaced by an aromatic/small amino acid pair, most frequently histidine and glycine. Receptors EDAR (CRD2), CD40, DR6, DcR3, LTβ-R, TNFR2, and RANK (CRD2) are all predicted to possess 1 or more of these larger, more B2-like modules.

The hOX40-OX40L Complex

Three copies of hOX40 receptor bind to the trimeric ligand to form the hOX40-OX40L complex. Each copy of hOX40 binds at a monomer-monomer interface on OX40L forming an extensive interface (2232 Å² hOX40-hOX40L; 2605 Å² hOX40-mOX40L) (FIG. 2, 5). This interface is larger than that seen in either the TNFR1-LT (Banner et al., Cell, 73:431-445 (1993)) or DR5-Apo21/TRAIL (Hymowitz et al., Mol Cell, 4:563-571 (1999); Mongkolsapaya et al., Nat Struct Biol, 6:1048-1053 (1999)) complexes and is split equally between each monomer (473 Å² for the F180 containing “right” monomer; 640 Å² for the N 166 containing “left” monomer”). Unexpectedly, hOX40 uses residues from CRD1 (e.g. T35, Y36, P37, S38, E45, V53, and R55) (355 Å²), CRD2 (e.g. S78, K79, P83, T85, W86, C87, and N88) (497 Å²) and CRD3 (e.g. Y119, V123, and D124)(167 Å²) to bind hOX40L.

In contrast, in the structures of TNFR1-LT and DR5-Apo2L, only residues from CRD2 and CRD3 are used with both CRDs making approximately equal contributions in binding ligand. OX40 uses the same general portions of CRD2 (the A1 loop and immediately following residues) and CRD3 (primarily the A1 loop) as used by TNFR1 or DR5 but makes additional contacts using CRD1. The ligand portion of this interface is even more discontinuous. Thirty one hOX40L residues from eleven different secondary structure elements including the unusual C-terminal tail contact receptor.

Overall, the OX40L-hOX40 complex structure spans ˜80 Å (FIG. 2). This distance, in conjunction with an ˜40 amino acid linker between the vestigial CRD4 and the OX40 transmembrane domain, suggests that the complex could connect cells whose surfaces are ˜100-150 Å apart. This distance is compatible with the dimensions of other ligand-receptor complexes which extend between antigen presenting cells and T cells such as the MHC-TCR complex and the B7-CD28 costimulatory complex (Schwartz et al., cited supra).

Comparison of the free mOX40L structure and the mOX40L-hOX40 complex indicate that there are no significant alterations in ligand conformation that are attributable to receptor binding. Two loops (the AA′ loop and residues 79-85 in the A′B′ loop) are significantly rearranged in the free vs. bound ligand structure; however, both of these loops in the free structure are involved in crystal packing contacts which likely drives the alterations in conformation.

Comparison of the human and murine complex structures helps explain why, despite their lack of high sequence similarity, both mOX40L and hOX40L bind hOX40 (FIG. 5). In general the murine and human OX40L surfaces are very distinct; of the 44 residues which bury any surface area in the mOX40L-hOX40 complex, only 26 are also involved in the hOX40L-OX40 interface. (See Tables 6 and 7) Of those twenty-six shared residues, only eleven are identical between mOX40L and hOX40L. The identical residues account for only ˜360 A² of the ligand interface (the total ligand buried surface area is ˜1200 A2). Six of the unshared residues are located in the C-terminal tail of mOX40L which is significantly longer in mOX40L than in hOX40L. Nonetheless, two critically important residues, F180 and N166, are conserved between human and murine OX40L and make similar contributions to binding receptor. Mutational analysis has identified both residues as being crucial for receptor affinity (see Example 2).

### Discussion

Both the structural and mutagenesis data show that the OX40-OX40L interface is not a contiguous patch with a single “hot-spot” but instead is distributed over the surface of hOX40L in at least two general areas. Mutagenesis data indicates that residues on both sides of the monomer-monomer interface are important for high affinity binding, and, further, that contacts made by three hOX40 CRD contribute to the binding affinity. The importance of F180 which is located in the hOX40L C-terminal tail indicates that this feature which is also present in murine and cyno OX40L sequences, is not just a structural oddity but that it has an important role in hOX40L function (FIG. 5b). Despite the extensive interface between hOX40 and hOX40L, the affinity of monomeric hOX40 for hOX40L was found to be ˜150 nM (Table II) which is in good agreement with values reported in the literature measured using other techniques (Al-Shamkhani et al., J Biol Chem, 272:5275-5282 (1997)), but is somewhat lower than had been reported for other TNF-multi-domain TNFR interactions However, since in vivo OX40L is expected to be membrane bound, other phenomena such as local clustering may lead to increased apparent affinity.

Two unusual features distinguish the hOX40-OX40L complex from other TNFSF-TNFRSF complexes: the unusual “open” OX40L trimer interface and the extensive contacts made by hOX40 CRD1 to OX40L (FIGS. 1,5). These features expand the TNFSF-TNFRSF repertoire of interactions. Until now, TNFSF ligand-multi CRD receptor interactions have all been interpreted using the rubric of the DR5-Apo2L and TNFR1-LT interactions which implied that only CRD2 and CRD3 were likely to contact ligand. In light of the OX40 complex structures reported here, it is possible that CRD1 plays a more important role in other TNFSF interactions than previously thought. For example, some disease causing mutations in EDA (Ectodysplasin) map to surface residues away from the areas where CRD2 and CRD3 from either EDAR or XEDAR are expected to bind (Hymowitz et al., Structure, 11:1513-1520 (2003)). Interestingly, these changes (A356D, R357P in the EDA BC loop and D298H, G299S in the EDA FG loop) map to areas of the EDA surface analogous to where OX40L contacts OX40 CRD1, indicating that EDAR and XEDAR (the receptors respectively for the EDA-A1 and EDA-A2 splice variants) may also use CRD1 to contact ligand.

The structures of the OX40L-hOX40 receptor complexes may shed new light on findings involving other OX40 interactions. For instance, Feline immunodeficiency virus, FIV, has recently been shown to require feline OX40 as a co-receptor for entry into T cells (de Parseval et al., Proc Natl Acad Sci USA, 101:13044-13049 (2004); Shimojima et al., Science, 303:1192-1195 (2004)). Elders et al have mapped the residues crucial for FIV interactions with feline OX40 to the tip of CRD1. Alteration of five residues in hOX40 CRD1 (H44S, R58G, S59D, N61D, V63K) is sufficient to confer FIV binding equivalent to that of feline OX40 (de Parseval et al., Nat Struct Mol Biol, 12:60-66 (2005)). These residues are distal from the receptor cell membrane and are not involved in binding OX40L consistent with biochemical data that soluble feline OX40L does not complete with FIV for binding feline OX40. However, the ability of feline OX40L to affect binding of FIV to feline OX40 may be different in vivo where OX40L is membrane-bound rather than soluble. The hOX40-hOX40L complex shows that the residues in OX40 CRD1 involved in binding FIV are likely to be very close to the ligand cell membrane (FIG. 2). The presence of the ligand cell membrane may sterically hinder FIV binding to OX40 in vivo.

In summary, the structure of the hOX40-OX40L complex shows that the ligand itself as well as the complex are more divergent that had been expected from sequence analysis. The OX40L trimer differs from that of other known TNFSF and distant homologs in its assembly while hOX40 receptor makes more extensive contacts including novel interactions mediated by CRD1 and the OX40L C-terminal tail that have not been seen in other TNFRSF members.

### Example 2

Mutations were made in the hOX40L to determine the effect of the mutations on binding to the hOX40 receptor. Several residues that were identified in the binding site in the hOX40L were substituted with alanine in order to determine the effect of these substitutions on the ability of the mutants to compete with wild-type ligand for binding to the receptor.

### Materials and Methods

### Alanine Scanning Mutagenesis

Single alanine mutations were made in the background of the hOX40L double glycosylation mutant at residues Q65, Q80, E123, T144, K146, D147, D162, H164, N166 and F180 using Quickchange site directed mutagenesis (Stratagene). Mutations at Q65, K146, D147, and H164 resulted in significantly decreased expression levels and were not purified or characterized. The remaining six alanine mutants were expressed and purified from insect cells as described above. Mutations were verified by DNA sequencing and mass spectrometry. Final buffer conditions were 150 mM NaCl, 20 mM Tris, pH 8.0.

### Competition ELISA Binding Experiments:

hOX40 was dialyzed into PBS and biotinylated using a 4 fold molar excess of biotin-NHS-LC. Serial dilutions of biotinylated hOX40 were then tested for binding to a plate coated with mOX40L. The dilution which gave approximately 50% saturating signal was used in the solution binding assay in which the biotinylated hOX40 was incubated with increasing concentrations of each hOX40L alanine mutant for 1 hour. The solutions were then transferred to mOX40L coated plates for 15 minutes to capture unbound receptor. Biotinylated hOX40 was detected with TMB substrate and the absorbance at 450 nM was measured. IC50 was calculated as the concentration of hOX40L alanine mutant in solution-binding stage that inhibited 50% of the receptor from binding to immobilized mOX40L.

### Results

### Functional Characterization of the OX40L-OX40 Complex

In order to determine which portions of the hOX40L surface contribute to receptor affinity, ten residues (Q65, Q80, E123, T144, K146, D147, D162, H164, N166 and F180) distributed over the surface were chosen for mutation to alanine in the context of the N90D, N114D double glycosylation mutant. Of these residues, four (Q65, K146, D147, and H164) resulted in severely diminished expression likely due to folding or secretion defects. The remaining six mutant proteins were shown by size exclusion chromatography to be properly assembled trimers.

The IC50 of these six mutants to compete for hOX40 was assessed in a competition ELISA assay (see Table II, FIG. 5b). Two of these substitutions, F180A in the C-terminal extension and N166A in the GH loop strand both resulted in undetectable binding for hOX40. Substitutions at Q80 (AA′ loop) and D162 (GH loop) had more moderate effects while the affinity of proteins with mutations at T144 (F strand) and E123 (DE loop) were essentially unchanged. The four residues which do affect ligand affinity for receptor do not form a compact region but instead are dispersed over the hOX40-hOX40L interface. The two 1 residues F180 and N166 are diagonally at opposite ends of the monomer-monomer interface. F180 interacts with a hydrophobic region on hOX40 CRD1 while N166 forms hydrogen bonds to the backbone of hOX40 residues W86 and C87 at the juncture of CRD2 and 3. F180 and N166 are conserved in murine OX40L (Y182 and N169) and make similar interactions in the hOX40-mOX40L complex. Q80, like F180, interacts with CRD1 but forms hydrogen bonds to the backbone of V53 rather than making hydrophobic contacts. D162 makes van der waals contacts near to where N166 binds. The murine equivalents of Q80 and D162 (N82 and H165) do not make similar interactions.

### Discussion

This mutational analysis shows that the binding energy in the hOX40-hOX40L interface is not concentrated in one location but is spread out to at least two areas, similar to what was seen for Apo2L/TRAIL interacting with DRS. However, the OX40-OX40L complex differs from the complexes formed by conventional TNFSF members with regard to the distribution of binding energy and the role of the ligand DE loop. In OX40L, unlike in the Apo2L/TRAIL or LT-receptor complexes, there is no significant hydrophobic contact between the DE loop and the A1 loop of CRD2. The one residue within the ligand DE loop which does extensively contact receptor, E123, does not contribute energetically to binding.

TABLE 1
Crystallographic Statistics

| Phasing mOX40L | Br Peak | Br High | Br Inflec | Br Low |
|---|---|---|---|---|
| Space Group | P6₃ | | | |
| Unit Cell (Å) | a = 75.0 c = 47.6 | | | |
| Wavelength (Å) | 0.9199 | 0.9050 | 0.9202 | 1.5418 |
| Max. Resolution (Å) | 2.0 | 2.2 | 2.0 | 2.6 |
| Completeness (%)^{a} | 99.8 (100) | 99.8 (100) | 99.8 (100) | 99.8 (100) |
| R_{sym}^{a,b} | 0.065 (0.42) | 0.067 (0.45) | 0.047 (0.33) | 0.045 (0.20) |
| Reflections measured^{c} | 91256 | 68648 | 91337 | 42450 |
| Reflections unique^{c} | 10438 | 7858 | 10452 | 4990 |
| Resolution for Phasing | 2.8 | 2.8 | 2.8 | 2.8 |
| Phasing power iso | 0.0 | 1.7 | 0.97 | 1.7 |
| Phasing power anom | 1.13 | 1.3 | 0.23 | 0.62 |
| FOM acentric, centric | 0.66, 0.62 | | | |

| | mOX40L | mOX40L-hOX40 | hOX40L-hOX40 |
|---|---|---|---|
| Data Collection | | | |
| Space Group | P6₃ | R32 | R32 |
| Resolution (Å)^{a} | 30-1.45 (1.50-1.45) | 30-2.0 (2.07-2.00) | 30-2.4 (2.49-2.40) |
| Unit cell constants (Å) | a = 74.5 c = 49.0 | a = 104.7 c = 478.2 | a = 111.9 c = 233.2 |
| R_{sym}^{a, b} | 0.068 (0.466) | 0.069 (0.272) | 0.055 (0.505) |
| No. observations | 388,637 | 312,989 | 135,176 |
| Unique reflections | 27,220 | 68,124 | 22,255 |
| Completeness (%)^{a} | 100 (100) | 98.6 (96.4) | 99.8 (100) |
| Asymmetric Unit | 1 ligand protomer | 2 ligand:receptor | 1 ligand:receptor |
| | | protomers | protomer |
| Refinement | | | |
| Resolution (Å) | 30-1.45 | 30-2.0 | 30-2.4 |
| Final R^{d}, R_{free} (%) | 16.5, 19.2 | 22.1, 24.7 | 20.8, 25.1 |
| No. solvent atoms | 108 | 181 | 87 |
| Rmsd bonds, angles (Å, °) | 0.007, 1.3 | 0.007, 1.0 | 0.007, 1.1 |
| Ramachandran Plot(%)^{e} | 90.4 8.7; 0.9; 0 | 92.1; 7.1; 0.9; 0 | 90.4 8.7; 0.9; 0 |

^{a}Numbers in parentheses refer to the highest resolution shell.
^{b}R_{sym} = Σ|I − <I>|/ΣI. <I> is the average intensity of symmetry related observations of a unique reflection.
^{c}Bijvoet reflections are kept separate in the Br statistics
^{d}R = Σ|F_{O} − F_{C}|/ΣF_{O}
^{e}Percentage of residues in the most favored, additionally allowed, generously allowed, and disallowed regions of a Ramachandran plot.

TABLE II
Effect of alanine mutations on hOX40L-hOX40 interactions in a
competition ELISA.

| | | Fold-change in IC50 |
|---|---|---|
| Protein | IC50 (nM) | (Mutant/D90D, N114D) |
| WT | 139 ± 43 | 2.2 |
| N90D, N114D | 62 ± 2 | 1.0 |
| F180A | N.D. | N.D. |
| N166A | N.D. | N.D. |
| Q80A | 537 ± 111 | 8.6 |
| D162A | 425 ± 73 | 6.9 |
| T144A | 139 ± 14 | 2.2 |
| E123A | 186 ± 5 | 3.0 |

IC50s are the average of three independent measurements.
All mutations are in the N90D, N114D background.
Fold-change values are relative to the IC50 of hOX40L containing the double mutation (N90D, N114D). Only values >3-fold different from the reference are considered significant.
N.D.: no detectable competition.

TABLE 3
mOX40L (SEQ ID NO: 1)

megegvqpld enlengsrpr fkwkktlrlv vsgikgagml
lcfiyvelql ssspakdppi qrlrgavtrc edgqlfissy
kneyqtmevq nnsvvikcdg lyiiylkgsf fqevkidlhf
redhnpisip mlndgrrivf tvvaslafkd kvyltvnapd
tlcehlqind gelivvqltp gycapegsyh stvnqvpl

TABLE 3b
(SEQ ID NO: 4)

SSSPAKDPPIQRLRGAVTRCEDGQLFISSYKNEYQTMEVQNNSVVIKC
51 64 98
DGLYIIYLKGSFFQEVKIDLHFREDHNPISIPMLNDGRRIVFTVVASLAFK
99
DKVYLTVNAPDTLCEHLQINDGELIVVQLTPGYCAPEGSYHSTVNQVPL
153 154 185 198

TABLE 4
hOX40 (SEQ ID NO: 2)

MCVGARRLGRGPCAALLLLGLGLSTVTGLHCVGDTYPSNDRCCHECRPG
NGMVSRCSRSQNTVCRPCGPGFYNDVVSSKPCKPCTWCNLRSGSERKQL
CTATQDTVCRCRAGTQPLDSYKPGVDCAPCPPGHFSPGDNQACKPWTNC
TLAGKHTLQPASNSSDAICEDRDPPATQPQETQGPPARPITVQPTEAWP
RTSQGPSTRPVEVPGGRAVAAILGLGLVLGLLGPLAILLALYLLRRDQR
LPPDAHKPPGGGSFRTPIQEEQADAHSTLAKI

TABLE 4b
(SEQ ID NO: 5)

LHCVGDTYPSNDRCCHECRPGNGMVSRCSRSQNTVCRPCGPGFYNDVV
29 76
SSKPCKPCTWCNLRSGSERKQLCTATQDTVCRCRAGTQPLDSYK
77 120
PGVDCAPCPPGHFSPGDNQACKPWTNCTLAGKHTLQPASNSSDAICEDRD
121 170

TABLE 5
hOX40L (SEQ ID NO: 3)

MERVQPLEENVGNAARPRFERNKLLLVASVIQGLGLLLCFTYICLHFS
ALQVSHRYPRIQSIKVQFTEYKKEKGFILTSQKEDEIMKVQNNSVIIN
CDGFYLISLKGYFSQEVNISLHYQKDEEPLFQLKKVRSVNSLMVASLT
YKDKVYLNVTTDNTSLDDFHVNGGELILIHQNPGEFCVL

TABLE 5b
(SEQ ID NO: 6)

QVSHRYPRIQSIKVQFTEYKKEKGFILTSQKEDEIMKVQNNSVIINCDGFY
51 58 101
LISLKGYFSQEVNISLHYQKDEEPLFQLKKVRSVNSLMVASLTYKDKVYLN
102
VTTDNTSLDDFHVNGGELILIHQNPGEFCVL
155 156 183

TABLE 5c
(SEQ ID NO: 7)

QVSHRYPRIQSIKVQFTEYKKEKGFILTSQKEDEIMKVQDNSVIINCDGFY
51 58 101
LISLKGYFSQEVDISLHYQKDEEPLFQLKKVRSVNSLMVASLTYKDKVYLN
102
VTTDNTSLDDFHVNGGELILIHQNPGEFCVL
155 156 183

TABLE 6

| Amino Acid | angstroms² | % of surface area buried |
|---|---|---|
| hOX40Ligand: | | |
| ASP A 98 | 28.00 | 70.00% of 40.00 |
| TYR A 119 | 18.00 | 33.96% of 53.00 |
| GLN A 120 | 2.00 | 2.94% of 68.00 |
| GLU A 123 | 63.00 | 66.32% of 95.00 |
| GLU A 124 | 30.00 | 22.39% of 134.00 |
| LYS A 130 | 19.00 | 13.48% of 141.00 |
| SER A 142 | 13.00 | 28.89% of 45.00 |
| THR A 144 | 51.00 | 76.12% of 67.00 |
| TYR A 145 | 42.00 | 46.15% of 91.00 |
| LYS A 146 | 91.00 | 60.67% of 150.00 |
| ASP A 147 | 4.00 | 66.67% of 6.00 |
| PHE A 180 | 85.00 | 72.03% of 118.00 |
| CYS A 181 | 1.00 | 2.13% of 47.00 |
| VAL A 182 | 26.00 | 48.15% of 54.00 |
| LYS A1063 | 14.00 | 17.72% of 79.00 |
| GLN A1065 | 56.00 | 61.54% of 91.00 |
| THR A1067 | 43.00 | 47.78% of 90.00 |
| THR A1078 | 9.00 | 13.85% of 65.00 |
| GLN A1080 | 53.00 | 43.44% of 122.00 |
| GLU A1082 | 63.00 | 49.22% of 128.00 |
| ASP A1083 | 28.00 | 19.05% of 147.00 |
| TYR A1108 | 31.00 | 29.25% of 106.00 |
| PHE A1109 | 2.00 | 40.00% of 5.00 |
| SER A1110 | 30.00 | 58.82% of 51.00 |
| ARG A1133 | 29.00 | 21.80% of 133.00 |
| ASP A1162 | 83.00 | 74.11% of 112.00 |
| HIS A1164 | 64.00 | 68.09% of 94.00 |
| VAL A1165 | 5.00 | 62.50% of 8.00 |
| ASN A1166 | 99.00 | 90.83% of 109.00 |
| GLY A1167 | 29.00 | 54.72% of 53.00 |
| GLY A1168 | 2.00 | 20.00% of 10.00 |
| CHAIN A DIFF- | 1113.0 | (7.55% of 14751.0 total |
| AREA: | | AREA for this chain) |
| hOX40Receptor: | | |
| GLY B 33 | 8.00 | 11.59% of 69.00 |
| THR B 35 | 12.00 | 44.44% of 27.00 |
| TYR B 36 | 22.00 | 95.65% of 23.00 |
| PRO B 37 | 62.00 | 68.89% of 90.00 |
| SER B 38 | 14.00 | 51.85% of 27.00 |
| GLU B 45 | 27.00 | 58.70% of 46.00 |
| MET B 52 | 11.00 | 35.48% of 31.00 |
| VAL B 53 | 62.00 | 75.61% of 82.00 |
| SER B 54 | 19.00 | 39.58% of 48.00 |
| ARG B 55 | 83.00 | 72.17% of 115.00 |
| ARG B 65 | 35.00 | 22.44% of 156.00 |
| PHE B 71 | 37.00 | 43.53% of 85.00 |
| ASP B 74 | 3.00 | 4.00% of 75.00 |
| VAL B 75 | 17.00 | 26.56% of 64.00 |
| SER B 78 | 24.00 | 82.76% of 29.00 |
| LYS B 79 | 98.00 | 72.59% of 135.00 |
| PRO B 80 | 22.00 | 23.40% of 94.00 |
| CYS B 81 | 6.00 | 20.00% of 30.00 |
| LYS B 82 | 25.00 | 32.47% of 77.00 |
| PRO B 83 | 47.00 | 65.28% of 72.00 |
| CYS B 84 | 5.00 | 20.83% of 24.00 |
| THR B 85 | 4.00 | 50.00% of 8.00 |
| TRP B 86 | 103.00 | 52.02% of 198.00 |
| CYS B 87 | 13.00 | 72.22% of 18.00 |
| ASN B 88 | 57.00 | 86.36% of 66.00 |
| LEU B 89 | 32.00 | 26.45% of 121.00 |
| ARG B 90 | 4.00 | 1.79% of 223.00 |
| ASP B 117 | 20.00 | 12.50% of 160.00 |
| TYR B 119 | 97.00 | 56.40% of 172.00 |
| LYS B 120 | 24.00 | 30.38% of 79.00 |
| PRO B 121 | 3.00 | 3.12% of 96.00 |
| VAL B 123 | 6.00 | 60.00% of 10.00 |
| ASP B 124 | 17.00 | 41.46% of 41.00 |
| ASP B 168 | 0.00 | 0.00% of 160.00 |
| CHAIN B DIFF- | 1019.0 | (11.05% of 9218.0 total |
| AREA: | | AREA for this chain) |
| TOTAL | 2132.0 | (8.89% of 23969.0 total |
| DIFF-AREA: | | AREA over all chains) |

TABLE 7

| Amino Acid | angstroms² | % Buried Surface Area |
|---|---|---|
| MOX40L | | |
| ASP A 99 | 19.00 | 86.36% of 22.00 |
| HIS A 119 | 13.00 | 65.00% of 20.00 |
| PHE A 120 | 4.00 | 20.00% of 20.00 |
| ARG A 121 | 22.00 | 27.50% of 80.00 |
| GLU A 122 | 12.00 | 9.52% of 126.00 |
| ASP A 123 | 24.00 | 20.00% of 120.00 |
| HIS A 124 | 8.00 | 14.29% of 56.00 |
| ASN A 125 | 69.00 | 51.49% of 134.00 |
| PRO A 126 | 24.00 | 47.06% of 51.00 |
| SER A 145 | 22.00 | 46.81% of 47.00 |
| LEU A 146 | 2.00 | 40.00% of 5.00 |
| ALA A 147 | 32.00 | 84.21% of 38.00 |
| PHE A 148 | 25.00 | 48.08% of 52.00 |
| LYS A 149 | 51.00 | 61.45% of 83.00 |
| ASP A 150 | 14.00 | 66.67% of 21.00 |
| TYR A 153 | 1.00 | 3.23% of 31.00 |
| TYR A 182 | 88.00 | 77.88% of 113.00 |
| ALA A 184 | 1.00 | 20.00% of 5.00 |
| PRO A 185 | 18.00 | 21.18% of 85.00 |
| GLY A 187 | 15.00 | 20.55% of 73.00 |
| SER A 188 | 68.00 | 91.89% of 74.00 |
| TYR A 189 | 53.00 | 55.21% of 96.00 |
| HIS A 190 | 47.00 | 28.83% of 163.00 |
| SER A 191 | 18.00 | 11.92% of 151.00 |
| ARG A1064 | 16.00 | 29.63% of 54.00 |
| ALA A1066 | 15.00 | 100.00% of 15.00 |
| THR A1068 | 54.00 | 63.53% of 85.00 |
| ARG A1069 | 6.00 | 5.88% of 102.00 |
| SER A1078 | 19.00 | 43.18% of 44.00 |
| TYR A1080 | 71.00 | 78.89% of 90.00 |
| LYS A1081 | 47.00 | 26.55% of 177.00 |
| ASN A1082 | 55.00 | 68.75% of 80.00 |
| GLU A1083 | 70.00 | 46.36% of 151.00 |
| PHE A1111 | 111.00 | 86.72% of 128.00 |
| GLN A1112 | 22.00 | 31.43% of 70.00 |
| LEU A1166 | 2.00 | 25.00% of 8.00 |
| GLN A1167 | 52.00 | 48.60% of 107.00 |
| ILE A1168 | 5.00 | 62.50% of 8.00 |
| ASN A1169 | 68.00 | 82.93% of 82.00 |
| ASP A1170 | 2.00 | 2.06% of 97.00 |
| GLY A1171 | 7.00 | 53.85% of 13.00 |
| CHAIN A DIFF- | 1272.0 | (8.69% of 14633.0 total |
| AREA: | | AREA for this chain) |
| HOX40 receptor | | |
| LEU B 29 | 28.00 | 20.44% of 137.00 |
| HIS B 30 | 62.00 | 38.51% of 161.00 |
| CYS B 31 | 28.00 | 96.55% of 29.00 |
| VAL B 32 | 19.00 | 17.59% of 108.00 |
| GLY B 33 | 38.00 | 55.88% of 68.00 |
| THR B 35 | 15.00 | 46.88% of 32.00 |
| TYR B 36 | 17.00 | 100.00% of 17.00 |
| PRO B 37 | 91.00 | 100.00% of 91.00 |
| SER B 38 | 14.00 | 50.00% of 28.00 |
| GLU B 45 | 28.00 | 57.14% of 49.00 |
| MET B 52 | 13.00 | 86.67% of 15.00 |
| VAL B 53 | 64.00 | 65.31% of 98.00 |
| SER B 54 | 21.00 | 60.00% of 35.00 |
| ARG B 55 | 109.00 | 91.60% of 119.00 |
| ARG B 65 | 12.00 | 7.27% of 165.00 |
| GLY B 70 | 3.00 | 6.98% of 43.00 |
| PHE B 71 | 47.00 | 51.65% of 91.00 |
| VAL B 75 | 19.00 | 24.36% of 78.00 |
| VAL B 76 | 15.00 | 28.30% of 53.00 |
| SER B 77 | 1.00 | 6.67% of 15.00 |
| SER B 78 | 18.00 | 51.43% of 35.00 |
| LYS B 79 | 96.00 | 64.00% of 150.00 |
| PRO B 80 | 71.00 | 74.74% of 95.00 |
| CYS B 81 | 11.00 | 42.31% of 26.00 |
| LYS B 82 | 1.00 | 1.25% of 80.00 |
| PRO B 83 | 54.00 | 62.07% of 87.00 |
| CYS B 84 | 13.00 | 48.15% of 27.00 |
| THR B 85 | 13.00 | 36.11% of 36.00 |
| TRP B 86 | 136.00 | 62.67% of 217.00 |
| CYS B 87 | 12.00 | 44.44% of 27.00 |
| ASN B 88 | 34.00 | 54.84% of 62.00 |
| LEU B 89 | 3.00 | 2.50% of 120.00 |
| ARG B 90 | 73.00 | 31.74% of 230.00 |
| ASP B 117 | 13.00 | 16.46% of 79.00 |
| TYR B 119 | 106.00 | 53.54% of 198.00 |
| LYS B 120 | 23.00 | 29.49% of 78.00 |
| VAL B 123 | 7.00 | 43.75% of 16.00 |
| ASP B 124 | 5.00 | 7.04% of 71.00 |
| CYS B 166 | 0.00 | 0.00% of 75.00 |
| CHAIN B DIFF- | 1333.0 | (14.29% of 9328.0 total |
| AREA: | | AREA for this chain) |
| TOTAL | 2605.0 | (10.87% of 23961.0 total |
| DIFF-AREA: | | AREA over all chains) |

TABLE 8

| | | |
|---|---|---|
| HEADER | ---- | XX-XXX-XX xxxx |
| COMPND | --- | |

| | | | |
|---|---|---|---|
| REMARK | 3 | | |
| REMARK | 3 | REFINEMENT. | |
| REMARK | 3 | PROGRAM | : REFMAC 5.2.0005 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) : | 1.45 |
| REMARK | 3 | RESOLUTION RANGE LOW | (ANGSTROMS) : | 30.00 |
| REMARK | 3 | DATA CUTOFF | (SIGMA(F)) : | NONE |
| REMARK | 3 | COMPLETENESS FOR RANGE | (%) : | 97.62 |
| REMARK | 3 | NUMBER OF REFLECTIONS | : | 23933 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.16765 |
| REMARK | 3 | R VALUE | (WORKING SET) | : 0.16505 |

| REMARK | 3 | FREE R VALUE | | : 0.19220 |
|---|---|---|---|---|
| REMARK | 3 | FREE R VALUE TEST SET SIZE | (%) | : 9.9 |

| | | | |
|---|---|---|---|
| REMARK | 3 | FREE R VALUE TEST SET COUNT | : 2635 |
| REMARK | 3 | | |
| REMARK | 3 | FIT IN THE HIGHEST RESOLUTION BIN. | |

| REMARK | 3 | TOTAL NUMBER OF BINS USED | | : 25 |
|---|---|---|---|---|
| REMARK | 3 | BIN RESOLUTION RANGE HIGH | | : 1.450 |
| REMARK | 3 | BIN RESOLUTION RANGE LOW | | : 1.480 |
| REMARK | 3 | REFLECTION IN BIN | (WORKING SET) | : 1466 |
| REMARK | 3 | BIN COMPLETENESS | (WORKING + TEST) (%) | : 100.00 |

| | | | | |
|---|---|---|---|---|
| REMARK | 3 | BIN R VALUE | (WORKING SET) | : 0.159 |

| | | | |
|---|---|---|---|
| REMARK | 3 | BIN FREE R VALUE SET COUNT | : 164 |
| REMARK | 3 | BIN FREE R VALUE | : 0.226 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS : 1152 | | |
| REMARK | 3 | | | |
| REMARK | 3 | B VALUES. | | |
| REMARK | 3 | FROM WILSON PLOT | (A**2) | : NULL |

| | | | | |
|---|---|---|---|---|
| REMARK | 3 | MEAN B VALUE | (OVERALL, A**2) | : 17.786 |

| REMARK | 3 | OVERALL ANISOTROPIC B VALUE. | |
|---|---|---|---|
| REMARK | 3 | B11 (A**2) : | −0.20 |
| REMARK | 3 | B22 (A**2) : | −0.20 |
| REMARK | 3 | B33 (A**2) : | 0.30 |
| REMARK | 3 | B12 (A**2) : | −0.10 |
| REMARK | 3 | B13 (A**2) : | 0.00 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.073 |
| REMARK | 3 | ESU BASED ON FREE R VALUE | (A): | 0.064 |
| REMARK | 3 | ESU BASED ON MAXIMUM LIKELIHOOD | (A): | 0.035 |
| REMARK | 3 | ESU FOR B VALUES BASED ON MAXIMUM LIKELIHOOD | (A**2): | 1.935 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.969 |
| REMARK | 3 | CORRELATION COEFFICIENT FO-FC FREE | : 0.962 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 1072; | 0.007; | 0.022 |
| REMARK | 3 | BOND LENGTHS OTHERS | (A): | 985; | 0.001; | 0.020 |
| REMARK | 3 | BOND ANGLES REFINED ATOMS | (DEGREES): | 1461; | 1.275; | 1.981 |
| REMARK | 3 | BOND ANGLES OTHERS | (DEGREES): | 2288; | 0.753; | 3.000 |
| REMARK | 3 | TORSION ANGLES, PERIOD 1 | (DEGREES): | 127; | 7.387; | 5.000 |
| REMARK | 3 | TORSION ANGLES, PERIOD 2 | (DEGREES): | 49; | 31.155; | 24.490 |
| REMARK | 3 | TORSION ANGLES, PERIOD 3 | (DEGREES): | 182; | 12.473; | 15.000 |
| REMARK | 3 | TORSION ANGLES, PERIOD 4 | (DEGREES): | 6; | 16.943; | 15.000 |
| REMARK | 3 | CHIRAL-CENTER RESTRAINTS | (A**3): | 174; | 0.085; | 0.200 |
| REMARK | 3 | GENERAL PLANES REFINED ATOMS | (A): | 1149; | 0.004; | 0.020 |
| REMARK | 3 | GENERAL PLANES OTHERS | (A): | 205; | 0.001; | 0.020 |
| REMARK | 3 | NON-BONDED CONTACTS REFINED ATOMS | (A): | 152; | 0.189; | 0.200 |
| REMARK | 3 | NON-BONDED CONTACTS OTHERS | (A): | 983; | 0.180; | 0.200 |
| REMARK | 3 | NON-BONDED TORSION REFINED ATOMS | (A): | 522; | 0.171; | 0.200 |
| REMARK | 3 | NON-BONDED TORSION OTHERS | (A): | 663; | 0.079; | 0.200 |
| REMARK | 3 | H-BOND (X...Y) REFINED ATOMS | (A): | 76; | 0.171; | 0.200 |
| REMARK | 3 | H-BOND (X...Y) OTHERS | (A): | 1; | 0.038; | 0.200 |
| REMARK | 3 | SYMMETRY VDW REFINED ATOMS | (A): | 14; | 0.203; | 0.200 |
| REMARK | 3 | SYMMETRY VDW OTHERS | (A): | 46; | 0.256; | 0.200 |
| REMARK | 3 | SYMMETRY H-BOND REFINED ATOMS | (A): | 17; | 0.207; | 0.200 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 838; | 2.794; | 2.500 |
| REMARK | 3 | MAIN-CHAIN BOND OTHER ATOMS | (A**2): | 258; | 1.059; | 2.500 |
| REMARK | 3 | MAIN-CHAIN ANGLE REFINED ATOMS | (A**2): | 1061; | 3.295; | 5.000 |
| REMARK | 3 | SIDE-CHAIN BOND REFINED ATOMS | (A**2): | 486; | 3.115; | 2.500 |
| REMARK | 3 | SIDE-CHAIN ANGLE REFINED ATOMS | (A**2): | 400; | 3.953; | 5.000 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | 2436; | 1.822; | 3.000 |
| REMARK | 3 | SPHERICITY; FREE ATOMS | (A**2): | 103; | 7.292; | 3.000 |
| REMARK | 3 | SPHERICITY; BONDED ATOMS | (A**2): | 2034; | 2.679; | 3.000 |
| REMARK | 3 | | | | | |
| REMARK | 3 | NCS RESTRAINTS STATISTICS | | | | |
| REMARK | 3 | NUMBER OF NCS GROUPS : NULL | | | | |
| REMARK | 3 | | | | | |
| REMARK | 3 | | | | | |
| REMARK | 3 | TLS DETAILS | | | | |
| REMARK | 3 | NUMBER OF TLS GROUPS : 1 | | | | |

| REMARK | 3 | ATOM RECORD CONTAINS RESIDUAL B FACTORS ONLY | | | |
|---|---|---|---|---|---|
| REMARK | 3 | | | | |
| REMARK | 3 | TLS GROUP : 1 | | | |
| REMARK | 3 | NUMBER OF COMPONENTS GROUP : 1 | | | |
| REMARK | 3 | COMPONENTS | C SSSEQI | TO | C SSSEQI |
| REMARK | 3 | RESIDUE RANGE: | F 58 | | F 201 |

| | | | | | |
|---|---|---|---|---|---|
| REMARK | 3 | ORIGIN FOR THE GROUP (A): | −8.9097 | 54.0568 | 32.7820 |

| REMARK | 3 | T TENSOR | | | | | |
|---|---|---|---|---|---|---|---|
| REMARK | 3 | T11: | −0.0281 | T22: | −0.0606 | | |
| REMARK | 3 | T33: | −0.0220 | T12: | 0.0006 | | |
| REMARK | 3 | T13: | −0.0113 | T23: | −0.0169 | | |
| REMARK | 3 | L TENSOR | | | | | |
| REMARK | 3 | L11: | 1.2563 | L22: | 1.8668 | | |
| REMARK | 3 | L33: | 1.6017 | L12: | 0.1382 | | |
| REMARK | 3 | L13: | 0.0472 | L23: | 1.0084 | | |
| REMARK | 3 | S TENSOR | | | | | |
| REMARK | 3 | S11: | 0.0001 | S12: | −0.0610 | S13: | 0.1595 |
| REMARK | 3 | S21: | 0.0766 | S22: | −0.0924 | S23: | 0.1510 |
| REMARK | 3 | S31: | −0.0310 | S32: | −0.1332 | S33: | 0.0923 |
| REMARK | 3 | | | | | | |
| REMARK | 3 | | | | | | |

| REMARK | 3 | BULK SOLVENT MODELLING. | |
|---|---|---|---|
| REMARK | 3 | METHOD USED : MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |
| REMARK | 3 | | |

| REMARK | 3 | OTHER REFINEMENT REMARKS: | | | | | |
|---|---|---|---|---|---|---|---|
| REMARK | 3 | HYDROGENS HAVE BEEN ADDED IN THE RIDING POSITIONS | | | | | |
| REMARK | 3 | | | | | | |
| SSBOND | 1 | CYS | F | 70 | CYS | F | 163 |
| SSBOND | 2 | CYS | F | 98 | CYS | F | 183 |

| | | | | | | | | | | |
|---|---|---|---|---|---|---|---|---|---|---|
| LINK | C1 | NAG | F | 200 | 1.439 | ND2 | ASN | F | 91 | NAG-ASN |

| | | | | | |
|---|---|---|---|---|---|
| MODRES | NAG | F | 200 | NAG-b-D | RENAME |

| | | | | | | | |
|---|---|---|---|---|---|---|---|
| CRYST1 | 74.149 | 74.149 | 48.815 | 90.00 | 90.00 | 120.00 | P 63 |

| | | | | |
|---|---|---|---|---|
| SCALE1 | 0.013486 | 0.007786 | 0.000000 | 0.00000 |
| SCALE2 | 0.000000 | 0.015573 | 0.000000 | 0.00000 |
| SCALE3 | 0.000000 | 0.000000 | 0.020486 | 0.00000 |

| | | | | | | | | | | | | |
|---|---|---|---|---|---|---|---|---|---|---|---|---|
| ATOM | 1 | N | PRO | F | 58 | −8.814 | 50.033 | 10.354 | 1.00 | 34.33 | | N |
| ANISOU | 1 | N | PRO | F | 58 | 4296 | 4377 | 4371 | −79 | −109 | −33 | N |
| ATOM | 2 | CA | PRO | F | 58 | −8.301 | 50.948 | 11.372 | 1.00 | 31.11 | | C |
| ANISOU | 2 | CA | PRO | F | 58 | 3802 | 4128 | 3890 | −50 | −120 | 59 | C |
| ATOM | 4 | CB | PRO | F | 58 | −9.448 | 51.015 | 12.391 | 1.00 | 33.84 | | C |
| ANISOU | 4 | CB | PRO | F | 58 | 4179 | 4514 | 4162 | 4 | −40 | −25 | C |
| ATOM | 7 | CG | PRO | F | 58 | −10.672 | 50.737 | 11.585 | 1.00 | 35.43 | | C |
| ANISOU | 7 | CG | PRO | F | 58 | 4357 | 4583 | 4521 | −38 | −82 | −6 | C |
| ATOM | 10 | CD | PRO | F | 58 | −10.245 | 49.736 | 10.549 | 1.00 | 36.08 | | C |
| ANISOU | 10 | CD | PRO | F | 58 | 4457 | 4653 | 4598 | −9 | −14 | 3 | C |
| ATOM | 13 | C | PRO | F | 58 | −7.016 | 50.459 | 12.046 | 1.00 | 28.59 | | C |
| ANISOU | 13 | C | PRO | F | 58 | 3738 | 3757 | 3365 | −123 | −55 | 22 | C |
| ATOM | 14 | O | PRO | F | 58 | −6.696 | 49.268 | 11.996 | 1.00 | 31.80 | | O |
| ANISOU | 14 | O | PRO | F | 58 | 4229 | 4050 | 3803 | −258 | 60 | −12 | O |
| ATOM | 17 | N | PRO | F | 59 | −6.284 | 51.377 | 12.689 | 1.00 | 24.78 | | N |
| ANISOU | 17 | N | PRO | F | 59 | 3234 | 3293 | 2889 | −77 | 41 | 234 | N |
| ATOM | 18 | CA | PRO | F | 59 | −5.044 | 51.002 | 13.358 | 1.00 | 22.83 | | C |
| ANISOU | 18 | CA | PRO | F | 59 | 3021 | 3041 | 2610 | −45 | −5 | 142 | C |
| ATOM | 20 | CB | PRO | F | 59 | −4.402 | 52.353 | 13.664 | 1.00 | 23.26 | | C |
| ANISOU | 20 | CB | PRO | F | 59 | 3076 | 2918 | 2841 | −13 | 78 | 135 | C |
| ATOM | 23 | CG | PRO | F | 59 | −5.545 | 53.283 | 13.800 | 1.00 | 25.77 | | C |
| ANISOU | 23 | CG | PRO | F | 59 | 3249 | 3407 | 3134 | −20 | 152 | −11 | C |
| ATOM | 26 | CD | PRO | F | 59 | −6.588 | 52.809 | 12.849 | 1.00 | 25.39 | | C |
| ANISOU | 26 | CD | PRO | F | 59 | 3168 | 3378 | 3100 | 29 | 162 | 98 | C |
| ATOM | 29 | C | PRO | F | 59 | −5.311 | 50.248 | 14.657 | 1.00 | 20.93 | | C |
| ANISOU | 29 | C | PRO | F | 59 | 2487 | 3005 | 2458 | −43 | 1 | 84 | C |
| ATOM | 30 | O | PRO | F | 59 | −6.440 | 50.240 | 15.146 | 1.00 | 21.96 | | O |
| ANISOU | 30 | O | PRO | F | 59 | 2459 | 3131 | 2750 | −69 | −106 | 127 | O |
| ATOM | 31 | N | ILE | F | 60 | −4.277 | 49.624 | 15.214 | 1.00 | 18.99 | | N |
| ANISOU | 31 | N | ILE | F | 60 | 2512 | 2563 | 2138 | 85 | 86 | 13 | N |
| ATOM | 32 | CA | ILE | F | 60 | −4.388 | 49.070 | 16.564 | 1.00 | 17.63 | | C |
| ANISOU | 32 | CA | ILE | F | 60 | 2205 | 2380 | 2113 | −87 | −2 | 27 | C |
| ATOM | 34 | CB | ILE | F | 60 | −3.033 | 48.528 | 17.092 | 1.00 | 18.26 | | C |
| ANISOU | 34 | CB | ILE | F | 60 | 2239 | 2427 | 2271 | −66 | −40 | −33 | C |
| ATOM | 36 | CG1 | ILE | F | 60 | −2.600 | 47.300 | 16.276 | 1.00 | 19.82 | | C |
| ANISOU | 36 | CG1 | ILE | F | 60 | 2234 | 2618 | 2679 | −73 | 92 | 50 | C |
| ATOM | 39 | CD1 | ILE | F | 60 | −1.163 | 46.887 | 16.504 | 1.00 | 21.47 | | C |
| ANISOU | 39 | CD1 | ILE | F | 60 | 2570 | 2920 | 2665 | 121 | −69 | 5 | C |
| ATOM | 43 | CG2 | ILE | F | 60 | −3.128 | 48.169 | 18.578 | 1.00 | 19.79 | | C |
| ANISOU | 43 | CG2 | ILE | F | 60 | 2229 | 2736 | 2551 | 54 | −203 | 101 | C |
| ATOM | 47 | C | ILE | F | 60 | −4.936 | 50.167 | 17.473 | 1.00 | 16.83 | | C |
| ANISOU | 47 | C | ILE | F | 60 | 2034 | 2219 | 2139 | −47 | −67 | 190 | C |
| ATOM | 48 | O | ILE | F | 60 | −4.466 | 51.307 | 17.464 | 1.00 | 16.48 | | O |
| ANISOU | 48 | O | ILE | F | 60 | 1992 | 2292 | 1976 | −175 | −121 | 63 | O |
| ATOM | 50 | N | GLN | F | 61 | −5.944 | 49.821 | 18.259 | 1.00 | 15.12 | | N |
| ANISOU | 50 | N | GLN | F | 61 | 1765 | 2046 | 1932 | −152 | −140 | −13 | N |
| ATOM | 51 | CA | GLN | F | 61 | −6.622 | 50.801 | 19.088 | 1.00 | 14.99 | | C |
| ANISOU | 51 | CA | GLN | F | 61 | 1933 | 1818 | 1942 | −43 | −92 | 144 | C |
| ATOM | 53 | CB | GLN | F | 61 | −8.043 | 50.334 | 19.367 | 1.00 | 15.53 | | C |
| ANISOU | 53 | CB | GLN | F | 61 | 1899 | 1959 | 2040 | −93 | −33 | −47 | C |
| ATOM | 56 | CG | GLN | F | 61 | −8.874 | 50.182 | 18.102 | 1.00 | 16.12 | | C |
| ANISOU | 56 | CG | GLN | F | 61 | 1858 | 2092 | 2172 | −110 | −95 | −7 | C |
| ATOM | 59 | CD | GLN | F | 61 | −9.270 | 51.515 | 17.503 | 1.00 | 16.91 | | C |
| ANISOU | 59 | CD | GLN | F | 61 | 2061 | 2320 | 2041 | −8 | −173 | 13 | C |
| ATOM | 60 | OE1 | GLN | F | 61 | −10.091 | 52.231 | 18.072 | 1.00 | 18.23 | | O |
| ANISOU | 60 | OE1 | GLN | F | 61 | 2087 | 2270 | 2566 | 31 | −82 | 78 | O |
| ATOM | 61 | NE2 | GLN | F | 61 | −8.679 | 51.861 | 16.365 | 1.00 | 17.40 | | N |
| ANISOU | 61 | NE2 | GLN | F | 61 | 1820 | 2470 | 2318 | 8 | −39 | 270 | N |
| ATOM | 64 | C | GLN | F | 61 | −5.864 | 51.014 | 20.381 | 1.00 | 15.33 | | C |
| ANISOU | 64 | C | GLN | F | 61 | 1920 | 1806 | 2098 | −106 | 31 | 48 | C |
| ATOM | 65 | O | GLN | F | 61 | −5.596 | 50.062 | 21.102 | 1.00 | 16.88 | | O |
| ANISOU | 65 | O | GLN | F | 61 | 2408 | 2024 | 1981 | −145 | −413 | 21 | O |
| ATOM | 67 | N | ARG | F | 62 | −5.496 | 52.261 | 20.657 | 1.00 | 12.95 | | N |
| ANISOU | 67 | N | ARG | F | 62 | 1542 | 1660 | 1718 | 60 | −20 | 29 | N |
| ATOM | 68 | CA | ARG | F | 62 | −4.871 | 52.582 | 21.931 | 1.00 | 14.89 | | C |
| ANISOU | 68 | CA | ARG | F | 62 | 1667 | 1891 | 2099 | 87 | −80 | −155 | C |
| ATOM | 70 | CB | ARG | F | 62 | −3.357 | 52.308 | 21.931 | 1.00 | 16.73 | | C |
| ANISOU | 70 | CB | ARG | F | 62 | 1869 | 2191 | 2295 | 182 | −69 | −88 | C |
| ATOM | 73 | CG | ARG | F | 62 | −2.504 | 53.254 | 21.113 | 1.00 | 18.96 | | C |
| ANISOU | 73 | CG | ARG | F | 62 | 2050 | 2603 | 2549 | 68 | −54 | −76 | C |
| ATOM | 76 | CD | ARG | F | 62 | −2.517 | 52.895 | 19.635 | 1.00 | 20.68 | | C |
| ANISOU | 76 | CD | ARG | F | 62 | 2307 | 2804 | 2744 | 44 | −65 | −9 | C |
| ATOM | 79 | NE | ARG | F | 62 | −1.282 | 53.303 | 18.976 | 1.00 | 19.35 | | N |
| ANISOU | 79 | NE | ARG | F | 62 | 2390 | 2415 | 2544 | 399 | −117 | 155 | N |
| ATOM | 81 | CZ | ARG | F | 62 | −0.973 | 53.011 | 17.718 | 1.00 | 19.21 | | C |
| ANISOU | 81 | CZ | ARG | F | 62 | 2558 | 2294 | 2445 | 286 | 22 | 147 | C |
| ATOM | 82 | NH1 | ARG | F | 62 | −1.782 | 52.283 | 16.964 | 1.00 | 18.86 | | N |
| ANISOU | 82 | NH1 | ARG | F | 62 | 2168 | 2446 | 2550 | −88 | 65 | 89 | N |
| ATOM | 85 | NH2 | ARG | F | 62 | 0.176 | 53.426 | 17.217 | 1.00 | 21.10 | | N |
| ANISOU | 85 | NH2 | ARG | F | 62 | 2316 | 2781 | 2918 | −64 | 8 | 118 | N |
| ATOM | 88 | C | ARG | F | 62 | −5.129 | 53.999 | 22.368 | 1.00 | 13.29 | | C |
| ANISOU | 88 | C | ARG | F | 62 | 1674 | 1637 | 1737 | −77 | −71 | −64 | C |
| ATOM | 89 | O | ARG | F | 62 | −5.370 | 54.904 | 21.557 | 1.00 | 13.99 | | O |
| ANISOU | 89 | O | ARG | F | 62 | 1759 | 1734 | 1820 | 27 | −44 | 77 | O |
| ATOM | 91 | N | LEU | F | 63 | −5.053 | 54.174 | 23.675 | 1.00 | 15.59 | | N |
| ANISOU | 91 | N | LEU | F | 63 | 2245 | 1874 | 1803 | 69 | −78 | 175 | N |
| ATOM | 92 | CA | LEU | F | 63 | −5.120 | 55.475 | 24.284 | 1.00 | 15.28 | | C |
| ANISOU | 92 | CA | LEU | F | 63 | 1992 | 1862 | 1949 | 30 | −48 | 1 | C |
| ATOM | 94 | CB | LEU | F | 63 | −6.566 | 55.818 | 24.626 | 1.00 | 18.62 | | C |
| ANISOU | 94 | CB | LEU | F | 63 | 2357 | 2269 | 2448 | 109 | −120 | −124 | C |
| ATOM | 97 | CG | LEU | F | 63 | −6.689 | 57.230 | 25.208 | 1.00 | 23.64 | | C |
| ANISOU | 97 | CG | LEU | F | 63 | 3058 | 2953 | 2970 | 192 | 97 | −351 | C |
| ATOM | 99 | CD1 | LEU | F | 63 | −7.681 | 58.054 | 24.427 | 1.00 | 26.60 | | C |
| ANISOU | 99 | CD1 | LEU | F | 63 | 3629 | 3223 | 3254 | 193 | 158 | −95 | C |
| ATOM | 103 | CD2 | LEU | F | 63 | −6.969 | 57.208 | 26.706 | 1.00 | 24.00 | | C |
| ANISOU | 103 | CD2 | LEU | F | 63 | 2990 | 3066 | 3062 | −117 | 115 | −103 | C |
| ATOM | 107 | C | LEU | F | 63 | −4.268 | 55.452 | 25.542 | 1.00 | 15.13 | | C |
| ANISOU | 107 | C | LEU | F | 63 | 2123 | 1763 | 1861 | −53 | −74 | −42 | C |
| ATOM | 108 | O | LEU | F | 63 | −4.352 | 54.512 | 26.329 | 1.00 | 16.78 | | O |
| ANISOU | 108 | O | LEU | F | 63 | 2495 | 1960 | 1919 | −75 | −28 | −36 | O |
| ATOM | 110 | N | ARG | F | 64 | −3.458 | 56.495 | 25.735 | 1.00 | 14.74 | | N |
| ANISOU | 110 | N | ARG | F | 64 | 1891 | 1800 | 1910 | −64 | −39 | −104 | N |
| ATOM | 111 | CA | ARG | F | 64 | −2.756 | 56.693 | 27.004 | 1.00 | 15.20 | | C |
| ANISOU | 111 | CA | ARG | F | 64 | 1834 | 2026 | 1915 | −58 | −18 | 84 | C |
| ATOM | 113 | CB | ARG | F | 64 | −1.241 | 56.637 | 26.843 | 1.00 | 15.87 | | C |
| ANISOU | 113 | CB | ARG | F | 64 | 1910 | 2066 | 2055 | −55 | 129 | −23 | C |
| ATOM | 116 | CG | ARG | F | 64 | −0.478 | 56.847 | 28.166 | 1.00 | 17.00 | | C |
| ANISOU | 116 | CG | ARG | F | 64 | 1931 | 2237 | 2291 | 32 | 86 | −80 | C |
| ATOM | 119 | CD | ARG | F | 64 | 0.947 | 56.326 | 28.083 | 1.00 | 18.05 | | C |
| ANISOU | 119 | CD | ARG | F | 64 | 2150 | 2349 | 2356 | 89 | 25 | 77 | C |
| ATOM | 122 | NE | ARG | F | 64 | 1.701 | 57.179 | 27.181 | 1.00 | 17.85 | | N |
| ANISOU | 122 | NE | ARG | F | 64 | 2207 | 2343 | 2229 | 198 | −73 | 181 | N |
| ATOM | 124 | CZ | ARG | F | 64 | 2.462 | 58.210 | 27.535 | 1.00 | 15.63 | | C |
| ANISOU | 124 | CZ | ARG | F | 64 | 1710 | 2087 | 2140 | 350 | −94 | 90 | C |
| ATOM | 125 | NH1 | ARG | F | 64 | 2.651 | 58.547 | 28.802 | 1.00 | 18.66 | | N |
| ANISOU | 125 | NH1 | ARG | F | 64 | 2652 | 2136 | 2301 | −4 | −1 | 151 | N |
| ATOM | 128 | NH2 | ARG | F | 64 | 3.066 | 58.906 | 26.593 | 1.00 | 16.50 | | N |
| ANISOU | 128 | NH2 | ARG | F | 64 | 1955 | 2149 | 2162 | 161 | −65 | 170 | N |
| ATOM | 131 | C | ARG | F | 64 | −3.194 | 58.044 | 27.521 | 1.00 | 15.62 | | C |
| ANISOU | 131 | C | ARG | F | 64 | 1719 | 2141 | 2073 | −42 | 26 | 92 | C |
| ATOM | 132 | O | ARG | F | 64 | −2.877 | 59.066 | 26.922 | 1.00 | 17.48 | | O |
| ANISOU | 132 | O | ARG | F | 64 | 2304 | 1899 | 2438 | 13 | 217 | −21 | O |
| ATOM | 134 | N | GLY | F | 65 | −3.961 | 58.038 | 28.603 | 1.00 | 16.07 | | N |
| ANISOU | 134 | N | GLY | F | 65 | 2012 | 1927 | 2165 | 18 | 85 | 16 | N |
| ATOM | 135 | CA | GLY | F | 65 | −4.540 | 59.258 | 29.140 | 1.00 | 16.98 | | C |
| ANISOU | 135 | CA | GLY | F | 65 | 2195 | 2107 | 2148 | 196 | −36 | −87 | C |
| ATOM | 138 | C | GLY | F | 65 | −3.985 | 59.627 | 30.494 | 1.00 | 17.09 | | C |
| ANISOU | 138 | C | GLY | F | 65 | 2240 | 2142 | 2111 | 181 | 133 | −67 | C |
| ATOM | 139 | O | GLY | F | 65 | −3.574 | 58.766 | 31.266 | 1.00 | 17.37 | | O |
| ANISOU | 139 | O | GLY | F | 65 | 2560 | 2022 | 2015 | 124 | −155 | −52 | O |
| ATOM | 141 | N | ALA | F | 66 | −4.007 | 60.916 | 30.790 | 1.00 | 16.38 | | N |
| ANISOU | 141 | N | ALA | F | 66 | 2177 | 2051 | 1996 | 87 | 230 | −53 | N |
| ATOM | 142 | CA | ALA | F | 66 | −3.642 | 61.399 | 32.106 | 1.00 | 16.35 | | C |
| ANISOU | 142 | CA | ALA | F | 66 | 2001 | 2182 | 2029 | −114 | 170 | 2 | C |
| ATOM | 144 | CB | ALA | F | 66 | −3.226 | 62.850 | 32.038 | 1.00 | 17.14 | | C |
| ANISOU | 144 | CB | ALA | F | 66 | 2346 | 2093 | 2073 | −81 | 163 | −105 | C |
| ATOM | 148 | C | ALA | F | 66 | −4.859 | 61.239 | 32.997 | 1.00 | 17.26 | | C |
| ANISOU | 148 | C | ALA | F | 66 | 2236 | 2288 | 2032 | 93 | 261 | −127 | C |
| ATOM | 149 | O | ALA | F | 66 | −5.966 | 61.570 | 32.593 | 1.00 | 17.71 | | O |
| ANISOU | 149 | O | ALA | F | 66 | 2152 | 2556 | 2018 | 18 | 280 | 209 | O |
| ATOM | 151 | N | VAL | F | 67 | −4.646 | 60.733 | 34.203 | 1.00 | 16.06 | | N |
| ANISOU | 151 | N | VAL | F | 67 | 1769 | 2251 | 2080 | 158 | 187 | 52 | N |
| ATOM | 152 | CA | VAL | F | 67 | −5.707 | 60.630 | 35.195 | 1.00 | 15.78 | | C |
| ANISOU | 152 | CA | VAL | F | 67 | 2086 | 2182 | 1726 | 42 | 186 | −8 | C |
| ATOM | 154 | CB | VAL | F | 67 | −5.726 | 59.239 | 35.865 | 1.00 | 16.98 | | C |
| ANISOU | 154 | CB | VAL | F | 67 | 2154 | 2172 | 2125 | −41 | −26 | −86 | C |
| ATOM | 156 | CG1 | VAL | F | 67 | −6.832 | 59.156 | 36.887 | 1.00 | 18.14 | | C |
| ANISOU | 156 | CG1 | VAL | F | 67 | 2361 | 2302 | 2226 | −43 | 66 | −126 | C |
| ATOM | 160 | CG2 | VAL | F | 67 | −5.895 | 58.155 | 34.811 | 1.00 | 17.87 | | C |
| ANISOU | 160 | CG2 | VAL | F | 67 | 2158 | 2127 | 2504 | −67 | 174 | −53 | C |
| ATOM | 164 | C | VAL | F | 67 | −5.400 | 61.748 | 36.175 | 1.00 | 16.24 | | C |
| ANISOU | 164 | C | VAL | F | 67 | 2154 | 2080 | 1936 | 117 | 163 | 28 | C |
| ATOM | 165 | O | VAL | F | 67 | −4.469 | 61.650 | 36.961 | 1.00 | 16.41 | | O |
| ANISOU | 165 | O | VAL | F | 67 | 2050 | 2120 | 2064 | 87 | 174 | −73 | O |
| ATOM | 167 | N | THR | F | 68 | −6.176 | 62.829 | 36.088 | 1.00 | 16.02 | | N |
| ANISOU | 167 | N | THR | F | 68 | 1925 | 2093 | 2066 | 247 | 49 | −18 | N |
| ATOM | 168 | CA | THR | F | 68 | −5.739 | 64.134 | 36.582 | 1.00 | 17.34 | | C |
| ANISOU | 168 | CA | THR | F | 68 | 2233 | 2270 | 2083 | 99 | 117 | −51 | C |
| ATOM | 170 | CB | THR | F | 68 | −6.168 | 65.235 | 35.590 | 1.00 | 19.08 | | C |
| ANISOU | 170 | CB | THR | F | 68 | 2463 | 2314 | 2472 | 34 | 252 | 125 | C |
| ATOM | 172 | OG1 | THR | F | 68 | −7.584 | 65.174 | 35.377 | 1.00 | 18.27 | | O |
| ANISOU | 172 | OG1 | THR | F | 68 | 2409 | 2511 | 2021 | 41 | 22 | −6 | O |
| ATOM | 174 | CG2 | THR | F | 68 | −5.461 | 65.048 | 34.255 | 1.00 | 20.34 | | C |
| ANISOU | 174 | CG2 | THR | F | 68 | 2835 | 2534 | 2359 | −37 | 214 | −35 | C |
| ATOM | 178 | C | THR | F | 68 | −6.254 | 64.528 | 37.962 | 1.00 | 16.44 | | C |
| ANISOU | 178 | C | THR | F | 68 | 2136 | 2135 | 1975 | −7 | 74 | −63 | C |
| ATOM | 179 | O | THR | F | 68 | −5.555 | 65.214 | 38.718 | 1.00 | 17.65 | | O |
| ANISOU | 179 | O | THR | F | 68 | 1910 | 2641 | 2154 | −131 | −23 | −317 | O |
| ATOM | 181 | N | ARG | F | 69 | −7.485 | 64.131 | 38.263 | 1.00 | 15.75 | | N |
| ANISOU | 181 | N | ARG | F | 69 | 1971 | 2115 | 1895 | −74 | 54 | 7 | N |
| ATOM | 182 | CA | ARG | F | 69 | −8.193 | 64.572 | 39.461 | 1.00 | 15.68 | | C |
| ANISOU | 182 | CA | ARG | F | 69 | 2067 | 1965 | 1925 | −250 | −64 | −40 | C |
| ATOM | 184 | CB | ARG | F | 69 | −8.497 | 66.070 | 39.383 | 1.00 | 15.93 | | C |
| ANISOU | 184 | CB | ARG | F | 69 | 1999 | 2108 | 1944 | 29 | 177 | −171 | C |
| ATOM | 187 | CG | ARG | F | 69 | −9.393 | 66.483 | 38.214 | 1.00 | 18.29 | | C |
| ANISOU | 187 | CG | ARG | F | 69 | 2342 | 2234 | 2373 | 154 | −19 | −107 | C |
| ATOM | 190 | CD | ARG | F | 69 | −9.420 | 68.004 | 38.018 | 1.00 | 22.32 | | C |
| ANISOU | 190 | CD | ARG | F | 69 | 3045 | 2524 | 2910 | 133 | 25 | −63 | C |
| ATOM | 193 | NE | ARG | F | 69 | −8.227 | 68.496 | 37.313 | 1.00 | 28.14 | | N |
| ANISOU | 193 | NE | ARG | F | 69 | 3503 | 3635 | 3555 | −28 | 25 | 108 | N |
| ATOM | 195 | CZ | ARG | F | 69 | −7.138 | 69.021 | 37.881 | 1.00 | 28.71 | | C |
| ANISOU | 195 | CZ | ARG | F | 69 | 3662 | 3658 | 3586 | −84 | 18 | 95 | C |
| ATOM | 196 | NH1 | ARG | F | 69 | −7.042 | 69.172 | 39.201 | 1.00 | 28.01 | | N |
| ANISOU | 196 | NH1 | ARG | F | 69 | 3677 | 3453 | 3511 | −56 | 55 | 23 | N |
| ATOM | 199 | NH2 | ARG | F | 69 | −6.128 | 69.422 | 37.112 | 1.00 | 28.22 | | N |
| ANISOU | 199 | NH2 | ARG | F | 69 | 3348 | 3869 | 3505 | −70 | 98 | 25 | N |
| ATOM | 202 | C | ARG | F | 69 | −9.492 | 63.789 | 39.613 | 1.00 | 12.99 | | C |
| ANISOU | 202 | C | ARG | F | 69 | 1701 | 1699 | 1535 | −101 | −57 | −50 | C |
| ATOM | 203 | O | ARG | F | 69 | −9.860 | 63.008 | 38.737 | 1.00 | 14.41 | | O |
| ANISOU | 203 | O | ARG | F | 69 | 2035 | 1960 | 1478 | −249 | 39 | −192 | O |
| ATOM | 205 | N | CYS | F | 70 | −10.176 | 64.011 | 40.733 | 1.00 | 13.36 | | N |
| ANISOU | 205 | N | CYS | F | 70 | 1817 | 1707 | 1552 | −60 | 190 | −136 | N |
| ATOM | 206 | CA | CYS | F | 70 | −11.494 | 63.447 | 40.992 | 1.00 | 13.78 | | C |
| ANISOU | 206 | CA | CYS | F | 70 | 1946 | 1716 | 1572 | −176 | −136 | −180 | C |
| ATOM | 208 | CB | CYS | F | 70 | −11.444 | 62.572 | 42.246 | 1.00 | 14.09 | | C |
| ANISOU | 208 | CB | CYS | F | 70 | 1787 | 1762 | 1804 | −172 | −5 | 36 | C |
| ATOM | 211 | SG | CYS | F | 70 | −12.995 | 61.712 | 42.674 | 1.00 | 17.48 | | S |
| ANISOU | 211 | SG | CYS | F | 70 | 2245 | 2059 | 2335 | −262 | −32 | 254 | S |
| ATOM | 213 | C | CYS | F | 70 | −12.499 | 64.579 | 41.171 | 1.00 | 15.06 | | C |
| ANISOU | 213 | C | CYS | F | 70 | 1820 | 2084 | 1816 | −239 | −5 | 85 | C |
| ATOM | 214 | O | CYS | F | 70 | −12.214 | 65.552 | 41.860 | 1.00 | 13.74 | | O |
| ANISOU | 214 | O | CYS | F | 70 | 1983 | 1859 | 1377 | −275 | 56 | −6 | O |
| ATOM | 216 | N | GLU | F | 71 | −13.655 | 64.460 | 40.526 | 1.00 | 15.36 | | N |
| ANISOU | 216 | N | GLU | F | 71 | 2187 | 1974 | 1675 | −193 | −182 | −93 | N |
| ATOM | 217 | CA | GLU | F | 71 | −14.777 | 65.355 | 40.781 | 1.00 | 18.85 | | C |
| ANISOU | 217 | CA | GLU | F | 71 | 2403 | 2470 | 2286 | −61 | −15 | −67 | C |
| ATOM | 219 | CB | GLU | F | 71 | −14.849 | 66.473 | 39.750 | 1.00 | 21.60 | | C |
| ANISOU | 219 | CB | GLU | F | 71 | 2970 | 2684 | 2552 | 56 | 43 | −46 | C |
| ATOM | 222 | CG | GLU | F | 71 | −15.948 | 67.477 | 40.060 | 1.00 | 23.17 | | C |
| ANISOU | 222 | CG | GLU | F | 71 | 3033 | 2864 | 2905 | 89 | −77 | 38 | C |
| ATOM | 225 | CD | GLU | F | 71 | −16.050 | 68.564 | 39.024 | 1.00 | 25.93 | | C |
| ANISOU | 225 | CD | GLU | F | 71 | 3423 | 3124 | 3302 | 54 | −11 | 165 | C |
| ATOM | 226 | OE1 | GLU | F | 71 | −15.053 | 69.276 | 38.839 | 1.00 | 31.24 | | O |
| ANISOU | 226 | OE1 | GLU | F | 71 | 3853 | 4035 | 3982 | −171 | 95 | 245 | O |
| ATOM | 227 | OE2 | GLU | F | 71 | −17.125 | 68.715 | 38.407 | 1.00 | 30.01 | | O |
| ANISOU | 227 | OE2 | GLU | F | 71 | 3803 | 3904 | 3694 | −158 | −266 | 191 | O |
| ATOM | 228 | C | GLU | F | 71 | −16.073 | 64.565 | 40.751 | 1.00 | 18.90 | | C |
| ANISOU | 228 | C | GLU | F | 71 | 2421 | 2490 | 2267 | −37 | −71 | −80 | C |
| ATOM | 229 | O | GLU | F | 71 | −16.279 | 63.758 | 39.850 | 1.00 | 17.04 | | O |
| ANISOU | 229 | O | GLU | F | 71 | 2369 | 2127 | 1979 | −61 | −254 | −46 | O |
| ATOM | 231 | N | ASP | F | 72 | −16.935 | 64.815 | 41.738 | 1.00 | 18.71 | | N |
| ANISOU | 231 | N | ASP | F | 72 | 2362 | 2360 | 2384 | 75 | −20 | −60 | N |
| ATOM | 232 | CA | ASP | F | 72 | −18.229 | 64.142 | 41.848 | 1.00 | 18.22 | | C |
| ANISOU | 232 | CA | ASP | F | 72 | 2248 | 2391 | 2282 | 55 | 50 | −59 | C |
| ATOM | 234 | CB | ASP | F | 72 | −19.200 | 64.689 | 40.793 | 1.00 | 22.83 | | C |
| ANISOU | 234 | CB | ASP | F | 72 | 2878 | 2958 | 2839 | 231 | −50 | 62 | C |
| ATOM | 237 | CG | ASP | F | 72 | −19.453 | 66.174 | 40.947 | 1.00 | 27.66 | | C |
| ANISOU | 237 | CG | ASP | F | 72 | 3556 | 3426 | 3525 | 182 | −63 | 85 | C |
| ATOM | 238 | OD1 | ASP | F | 72 | −19.647 | 66.632 | 42.091 | 1.00 | 27.57 | | O |
| ANISOU | 238 | OD1 | ASP | F | 72 | 3646 | 3324 | 3503 | 417 | −127 | 0 | O |
| ATOM | 239 | OD2 | ASP | F | 72 | −19.459 | 66.885 | 39.921 | 1.00 | 33.79 | | O |
| ANISOU | 239 | OD2 | ASP | F | 72 | 4439 | 4194 | 4204 | 150 | −42 | 366 | O |
| ATOM | 240 | C | ASP | F | 72 | −18.122 | 62.616 | 41.733 | 1.00 | 18.15 | | C |
| ANISOU | 240 | C | ASP | F | 72 | 2402 | 2371 | 2120 | 34 | −50 | 52 | C |
| ATOM | 241 | O | ASP | F | 72 | −18.972 | 61.969 | 41.127 | 1.00 | 19.94 | | O |
| ANISOU | 241 | O | ASP | F | 72 | 2504 | 2551 | 2520 | 46 | −139 | 8 | O |
| ATOM | 243 | N | GLY | F | 73 | −17.073 | 62.047 | 42.320 | 1.00 | 15.68 | | N |
| ANISOU | 243 | N | GLY | F | 73 | 1913 | 2080 | 1965 | −80 | 16 | −156 | N |
| ATOM | 244 | CA | GLY | F | 73 | −16.909 | 60.601 | 42.363 | 1.00 | 15.91 | | C |
| ANISOU | 244 | CA | GLY | F | 73 | 2081 | 2132 | 1830 | −85 | 39 | −30 | C |
| ATOM | 247 | C | GLY | F | 73 | −16.345 | 59.957 | 41.108 | 1.00 | 16.51 | | C |
| ANISOU | 247 | C | GLY | F | 73 | 2323 | 2096 | 1851 | −52 | 33 | 68 | C |
| ATOM | 248 | O | GLY | F | 73 | −16.293 | 58.738 | 41.031 | 1.00 | 17.61 | | O |
| ANISOU | 248 | O | GLY | F | 73 | 2596 | 2063 | 2031 | −80 | 145 | 8 | O |
| ATOM | 250 | N | GLN | F | 74 | −15.919 | 60.764 | 40.136 | 1.00 | 15.15 | | N |
| ANISOU | 250 | N | GLN | F | 74 | 1950 | 2010 | 1796 | 56 | 5 | −76 | N |
| ATOM | 251 | CA | GLN | F | 74 | −15.341 | 60.238 | 38.905 | 1.00 | 14.99 | | C |
| ANISOU | 251 | CA | GLN | F | 74 | 1804 | 1931 | 1958 | −88 | 45 | 47 | C |
| ATOM | 253 | CB | GLN | F | 74 | −16.200 | 60.605 | 37.692 | 1.00 | 16.14 | | C |
| ANISOU | 253 | CB | GLN | F | 74 | 1978 | 2257 | 1897 | −64 | 0 | −24 | C |
| ATOM | 256 | CG | GLN | F | 74 | −15.687 | 59.981 | 36.395 | 1.00 | 17.47 | | C |
| ANISOU | 256 | CG | GLN | F | 74 | 2310 | 2346 | 1980 | −97 | −49 | −49 | C |
| ATOM | 259 | CD | GLN | F | 74 | −16.622 | 60.159 | 35.217 | 1.00 | 19.11 | | C |
| ANISOU | 259 | CD | GLN | F | 74 | 2266 | 2692 | 2302 | 100 | −50 | 8 | C |
| ATOM | 260 | OE1 | GLN | F | 74 | −17.592 | 60.923 | 35.269 | 1.00 | 22.97 | | O |
| ANISOU | 260 | OE1 | GLN | F | 74 | 2743 | 3171 | 2811 | 545 | 12 | 51 | O |
| ATOM | 261 | NE2 | GLN | F | 74 | −16.324 | 59.460 | 34.137 | 1.00 | 19.36 | | N |
| ANISOU | 261 | NE2 | GLN | F | 74 | 2670 | 2499 | 2186 | −89 | −190 | −262 | N |
| ATOM | 264 | C | GLN | F | 74 | −13.936 | 60.793 | 38.719 | 1.00 | 13.87 | | C |
| ANISOU | 264 | C | GLN | F | 74 | 1754 | 1856 | 1660 | −66 | 85 | −16 | C |
| ATOM | 265 | O | GLN | F | 74 | −13.695 | 61.984 | 38.925 | 1.00 | 15.30 | | O |
| ANISOU | 265 | O | GLN | F | 74 | 2035 | 1887 | 1890 | 13 | 120 | −58 | O |
| ATOM | 267 | N | LEU | F | 75 | −13.014 | 59.921 | 38.324 | 1.00 | 14.21 | | N |
| ANISOU | 267 | N | LEU | F | 75 | 1850 | 1783 | 1764 | −36 | −17 | −94 | N |
| ATOM | 268 | CA | LEU | F | 75 | −11.676 | 60.362 | 37.957 | 1.00 | 13.72 | | C |
| ANISOU | 268 | CA | LEU | F | 75 | 1720 | 1821 | 1673 | −199 | 47 | −70 | C |
| ATOM | 270 | CB | LEU | F | 75 | −10.636 | 59.247 | 38.069 | 1.00 | 15.19 | | C |
| ANISOU | 270 | CB | LEU | F | 75 | 1795 | 1828 | 2146 | −344 | −107 | 18 | C |
| ATOM | 273 | CG | LEU | F | 75 | −10.460 | 58.632 | 39.456 | 1.00 | 17.99 | | C |
| ANISOU | 273 | CG | LEU | F | 75 | 2276 | 2333 | 2225 | −216 | 55 | 126 | C |
| ATOM | 275 | CD1 | LEU | F | 75 | −9.285 | 57.694 | 39.498 | 1.00 | 18.25 | | C |
| ANISOU | 275 | CD1 | LEU | F | 75 | 2453 | 2203 | 2276 | −154 | −93 | 84 | C |
| ATOM | 279 | CD2 | LEU | F | 75 | −10.270 | 59.693 | 40.500 | 1.00 | 20.52 | | C |
| ANISOU | 279 | CD2 | LEU | F | 75 | 2803 | 2410 | 2583 | 54 | −94 | 40 | C |
| ATOM | 283 | C | LEU | F | 75 | −11.705 | 60.914 | 36.552 | 1.00 | 14.36 | | C |
| ANISOU | 283 | C | LEU | F | 75 | 1787 | 1845 | 1821 | −164 | 26 | −71 | C |
| ATOM | 284 | O | LEU | F | 75 | −12.272 | 60.311 | 35.645 | 1.00 | 17.12 | | O |
| ANISOU | 284 | O | LEU | F | 75 | 2202 | 2555 | 1746 | −562 | −69 | −366 | O |
| ATOM | 286 | N | PHE | F | 76 | −11.087 | 62.072 | 36.381 | 1.00 | 13.68 | | N |
| ANISOU | 286 | N | PHE | F | 76 | 1751 | 1688 | 1755 | −195 | 77 | −90 | N |
| ATOM | 287 | CA | PHE | F | 76 | −10.998 | 62.699 | 35.074 | 1.00 | 13.76 | | C |
| ANISOU | 287 | CA | PHE | F | 76 | 1601 | 1934 | 1691 | −96 | −38 | 33 | C |
| ATOM | 289 | CB | PHE | F | 76 | −10.774 | 64.208 | 35.220 | 1.00 | 18.02 | | C |
| ANISOU | 289 | CB | PHE | F | 76 | 2350 | 2272 | 2224 | −76 | −22 | 19 | C |
| ATOM | 292 | CG | PHE | F | 76 | −12.027 | 64.986 | 35.534 | 1.00 | 19.08 | | C |
| ANISOU | 292 | CG | PHE | F | 76 | 2493 | 2519 | 2237 | −78 | −4 | −85 | C |
| ATOM | 293 | CD1 | PHE | F | 76 | −13.123 | 64.387 | 36.150 | 1.00 | 19.74 | | C |
| ANISOU | 293 | CD1 | PHE | F | 76 | 2571 | 2557 | 2371 | 96 | 217 | −131 | C |
| ATOM | 295 | CE1 | PHE | F | 76 | −14.269 | 65.124 | 36.437 | 1.00 | 20.29 | | C |
| ANISOU | 295 | CE1 | PHE | F | 76 | 2514 | 2723 | 2471 | 33 | 6 | −75 | C |
| ATOM | 297 | CZ | PHE | F | 76 | −14.333 | 66.461 | 36.106 | 1.00 | 20.84 | | C |
| ANISOU | 297 | CZ | PHE | F | 76 | 2584 | 2783 | 2549 | 180 | 152 | −9 | C |
| ATOM | 299 | CE2 | PHE | F | 76 | −13.251 | 67.075 | 35.505 | 1.00 | 21.35 | | C |
| ANISOU | 299 | CE2 | PHE | F | 76 | 2701 | 2604 | 2807 | 117 | 79 | 164 | C |
| ATOM | 301 | CD2 | PHE | F | 76 | −12.104 | 66.335 | 35.219 | 1.00 | 22.26 | | C |
| ANISOU | 301 | CD2 | PHE | F | 76 | 2860 | 2808 | 2787 | 114 | 142 | 59 | C |
| ATOM | 303 | C | PHE | F | 76 | −9.877 | 62.062 | 34.267 | 1.00 | 15.24 | | C |
| ANISOU | 303 | C | PHE | F | 76 | 1830 | 2062 | 1898 | 26 | −84 | −101 | C |
| ATOM | 304 | O | PHE | F | 76 | −8.825 | 61.708 | 34.806 | 1.00 | 17.61 | | O |
| ANISOU | 304 | O | PHE | F | 76 | 2122 | 2710 | 1858 | 159 | 5 | −165 | O |
| ATOM | 306 | N | ILE | F | 77 | −10.128 | 61.905 | 32.973 | 1.00 | 15.50 | | N |
| ANISOU | 306 | N | ILE | F | 77 | 1866 | 2287 | 1737 | 84 | 40 | −32 | N |
| ATOM | 307 | CA | ILE | F | 77 | −9.175 | 61.315 | 32.048 | 1.00 | 15.38 | | C |
| ANISOU | 307 | CA | ILE | F | 77 | 1907 | 2203 | 1732 | 99 | −7 | −43 | C |
| ATOM | 309 | CB | ILE | F | 77 | −9.667 | 59.966 | 31.493 | 1.00 | 16.87 | | C |
| ANISOU | 309 | CB | ILE | F | 77 | 2093 | 2212 | 2105 | 64 | 230 | −87 | C |
| ATOM | 311 | CG1 | ILE | F | 77 | −9.947 | 58.986 | 32.640 | 1.00 | 18.70 | | C |
| ANISOU | 311 | CG1 | ILE | F | 77 | 2324 | 2295 | 2483 | 9 | 155 | 63 | C |
| ATOM | 314 | CD1 | ILE | F | 77 | −10.637 | 57.701 | 32.201 | 1.00 | 20.04 | | C |
| ANISOU | 314 | CD1 | ILE | F | 77 | 2607 | 2412 | 2594 | 74 | 161 | −13 | C |
| ATOM | 318 | CG2 | ILE | F | 77 | −8.636 | 59.387 | 30.506 | 1.00 | 18.52 | | C |
| ANISOU | 318 | CG2 | ILE | F | 77 | 2369 | 2575 | 2092 | 109 | 158 | −223 | C |
| ATOM | 322 | C | ILE | F | 77 | −8.991 | 62.281 | 30.897 | 1.00 | 18.04 | | C |
| ANISOU | 322 | C | ILE | F | 77 | 2493 | 2304 | 2056 | 69 | 166 | −77 | C |
| ATOM | 323 | O | ILE | F | 77 | −9.970 | 62.691 | 30.271 | 1.00 | 19.54 | | O |
| ANISOU | 323 | O | ILE | F | 77 | 2448 | 2878 | 2095 | −47 | 284 | 11 | O |
| ATOM | 325 | N | SER | F | 78 | −7.734 | 62.620 | 30.634 | 1.00 | 18.39 | | N |
| ANISOU | 325 | N | SER | F | 78 | 2444 | 2418 | 2123 | 151 | 127 | −31 | N |
| ATOM | 326 | CA | SER | F | 78 | −7.343 | 63.575 | 29.602 | 1.00 | 19.51 | | C |
| ANISOU | 326 | CA | SER | F | 78 | 2669 | 2490 | 2253 | 204 | 263 | 77 | C |
| ATOM | 328 | CB | SER | F | 78 | −6.552 | 64.719 | 30.249 | 1.00 | 22.44 | | C |
| ANISOU | 328 | CB | SER | F | 78 | 3169 | 2579 | 2778 | 23 | 230 | 90 | C |
| ATOM | 331 | OG | SER | F | 78 | −5.839 | 65.487 | 29.293 | 1.00 | 24.20 | | O |
| ANISOU | 331 | OG | SER | F | 78 | 3196 | 3167 | 2832 | −76 | 494 | −224 | O |
| ATOM | 333 | C | SER | F | 78 | −6.473 | 62.879 | 28.557 | 1.00 | 19.75 | | C |
| ANISOU | 333 | C | SER | F | 78 | 2696 | 2414 | 2392 | 339 | 251 | 125 | C |
| ATOM | 334 | O | SER | F | 78 | −5.622 | 62.068 | 28.902 | 1.00 | 21.81 | | O |
| ANISOU | 334 | O | SER | F | 78 | 2929 | 3126 | 2233 | 580 | 189 | 325 | O |
| ATOM | 336 | N | SER | F | 79 | −6.669 | 63.206 | 27.284 | 1.00 | 21.32 | | N |
| ANISOU | 336 | N | SER | F | 79 | 2696 | 2833 | 2569 | 192 | 215 | −1 | N |
| ATOM | 337 | CA | SER | F | 79 | −5.804 | 62.674 | 26.231 | 1.00 | 20.00 | | C |
| ANISOU | 337 | CA | SER | F | 79 | 2538 | 2708 | 2352 | 62 | 81 | −83 | C |
| ATOM | 339 | CB | SER | F | 79 | −6.546 | 62.644 | 24.889 | 1.00 | 23.79 | | C |
| ANISOU | 339 | CB | SER | F | 79 | 3073 | 3405 | 2561 | 87 | −9 | 59 | C |
| ATOM | 342 | OG | SER | F | 79 | −6.843 | 63.956 | 24.451 | 1.00 | 27.88 | | O |
| ANISOU | 342 | OG | SER | F | 79 | 3530 | 3628 | 3434 | −34 | −159 | −38 | O |
| ATOM | 344 | C | SER | F | 79 | −4.481 | 63.451 | 26.087 | 1.00 | 19.51 | | C |
| ANISOU | 344 | C | SER | F | 79 | 2348 | 2765 | 2298 | 68 | 87 | 47 | C |
| ATOM | 345 | O | SER | F | 79 | −3.704 | 63.159 | 25.176 | 1.00 | 18.96 | | O |
| ANISOU | 345 | O | SER | F | 79 | 2417 | 2839 | 1945 | 403 | −28 | 186 | O |
| ATOM | 347 | N | TYR | F | 80 | −4.221 | 64.411 | 26.986 | 1.00 | 18.72 | | N |
| ANISOU | 347 | N | TYR | F | 80 | 2280 | 2620 | 2212 | 93 | 232 | −9 | N |
| ATOM | 348 | CA | TYR | F | 80 | −2.985 | 65.209 | 26.973 | 1.00 | 19.32 | | C |
| ANISOU | 348 | CA | TYR | F | 80 | 2396 | 2480 | 2464 | 65 | 39 | −53 | C |
| ATOM | 350 | CB | TYR | F | 80 | −3.150 | 66.523 | 27.766 | 1.00 | 22.20 | | C |
| ANISOU | 350 | CB | TYR | F | 80 | 2920 | 2838 | 2677 | −34 | 61 | −72 | C |
| ATOM | 353 | CG | TYR | F | 80 | −1.922 | 67.434 | 27.727 | 1.00 | 23.19 | | C |
| ANISOU | 353 | CG | TYR | F | 80 | 2930 | 3124 | 2754 | −42 | 28 | 13 | C |
| ATOM | 354 | CD1 | TYR | F | 80 | −1.541 | 68.083 | 26.550 | 1.00 | 23.18 | | C |
| ANISOU | 354 | CD1 | TYR | F | 80 | 2967 | 2935 | 2903 | −35 | 46 | 68 | C |
| ATOM | 356 | CE1 | TYR | F | 80 | −0.417 | 68.903 | 26.511 | 1.00 | 22.74 | | C |
| ANISOU | 356 | CE1 | TYR | F | 80 | 2952 | 3004 | 2683 | 0 | −2 | 51 | C |
| ATOM | 358 | CZ | TYR | F | 80 | 0.332 | 69.089 | 27.657 | 1.00 | 23.56 | | C |
| ANISOU | 358 | CZ | TYR | F | 80 | 2995 | 3027 | 2928 | −43 | −4 | 46 | C |
| ATOM | 359 | OH | TYR | F | 80 | 1.446 | 69.895 | 27.633 | 1.00 | 23.19 | | O |
| ANISOU | 359 | OH | TYR | F | 80 | 2812 | 3278 | 2719 | −2 | 8 | 16 | O |
| ATOM | 361 | CE2 | TYR | F | 80 | −0.029 | 68.459 | 28.837 | 1.00 | 24.58 | | C |
| ANISOU | 361 | CE2 | TYR | F | 80 | 3093 | 3357 | 2887 | −94 | 20 | 39 | C |
| ATOM | 363 | CD2 | TYR | F | 80 | −1.147 | 67.643 | 28.867 | 1.00 | 24.38 | | C |
| ANISOU | 363 | CD2 | TYR | F | 80 | 3111 | 3155 | 2996 | −111 | −38 | 2 | C |
| ATOM | 365 | C | TYR | F | 80 | −1.793 | 64.412 | 27.493 | 1.00 | 20.18 | | C |
| ANISOU | 365 | C | TYR | F | 80 | 2404 | 2771 | 2493 | 119 | 40 | −80 | C |
| ATOM | 366 | O | TYR | F | 80 | −1.374 | 64.542 | 28.651 | 1.00 | 22.67 | | O |
| ANISOU | 366 | O | TYR | F | 80 | 2784 | 3163 | 2666 | 267 | 103 | −224 | O |
| ATOM | 368 | N | LYS | F | 81 | −1.264 | 63.577 | 26.611 | 1.00 | 18.78 | | N |
| ANISOU | 368 | N | LYS | F | 81 | 2140 | 2723 | 2273 | 20 | 15 | −74 | N |
| ATOM | 369 | CA | LYS | F | 81 | −0.049 | 62.807 | 26.843 | 1.00 | 17.48 | | C |
| ANISOU | 369 | CA | LYS | F | 81 | 2080 | 2476 | 2085 | 103 | −48 | −8 | C |
| ATOM | 371 | CB | LYS | F | 81 | −0.382 | 61.370 | 27.258 | 1.00 | 20.98 | | C |
| ANISOU | 371 | CB | LYS | F | 81 | 2645 | 2543 | 2781 | −31 | 61 | −3 | C |
| ATOM | 374 | CG | LYS | F | 81 | −0.846 | 61.200 | 28.699 | 1.00 | 22.98 | | C |
| ANISOU | 374 | CG | LYS | F | 81 | 2883 | 2871 | 2976 | −113 | 80 | 78 | C |
| ATOM | 377 | CD | LYS | F | 81 | 0.219 | 61.676 | 29.673 | 1.00 | 27.09 | | C |
| ANISOU | 377 | CD | LYS | F | 81 | 3314 | 3700 | 3279 | 33 | −17 | 30 | C |
| ATOM | 380 | CE | LYS | F | 81 | 0.115 | 61.023 | 31.013 | 1.00 | 28.43 | | C |
| ANISOU | 380 | CE | LYS | F | 81 | 3449 | 3820 | 3530 | −54 | −30 | −22 | C |
| ATOM | 383 | NZ | LYS | F | 81 | 1.183 | 61.575 | 31.887 | 1.00 | 29.24 | | N |
| ANISOU | 383 | NZ | LYS | F | 81 | 3437 | 4206 | 3465 | −105 | −267 | −69 | N |
| ATOM | 387 | C | LYS | F | 81 | 0.719 | 62.769 | 25.531 | 1.00 | 17.08 | | C |
| ANISOU | 387 | C | LYS | F | 81 | 1825 | 2641 | 2021 | 158 | 79 | 227 | C |
| ATOM | 388 | O | LYS | F | 81 | 0.126 | 62.916 | 24.449 | 1.00 | 16.54 | | O |
| ANISOU | 388 | O | LYS | F | 81 | 1697 | 2529 | 2057 | 323 | 45 | 202 | O |
| ATOM | 390 | N | ASN | F | 82 | 2.026 | 62.536 | 25.609 | 1.00 | 15.86 | | N |
| ANISOU | 390 | N | ASN | F | 82 | 1644 | 2454 | 1925 | 187 | −144 | 79 | N |
| ATOM | 391 | CA | ASN | F | 82 | 2.833 | 62.369 | 24.396 | 1.00 | 14.60 | | C |
| ANISOU | 391 | CA | ASN | F | 82 | 1677 | 2094 | 1774 | 21 | −153 | 153 | C |
| ATOM | 393 | CB | ASN | F | 82 | 4.308 | 62.690 | 24.648 | 1.00 | 14.67 | | C |
| ANISOU | 393 | CB | ASN | F | 82 | 1742 | 2087 | 1745 | 197 | −393 | 154 | C |
| ATOM | 396 | CG | ASN | F | 82 | 4.548 | 64.168 | 24.801 | 1.00 | 14.07 | | C |
| ANISOU | 396 | CG | ASN | F | 82 | 1536 | 2129 | 1680 | 39 | −39 | 124 | C |
| ATOM | 397 | OD1 | ASN | F | 82 | 4.588 | 64.903 | 23.817 | 1.00 | 13.53 | | O |
| ANISOU | 397 | OD1 | ASN | F | 82 | 1480 | 2247 | 1412 | 383 | −301 | 261 | O |
| ATOM | 398 | ND2 | ASN | F | 82 | 4.687 | 64.623 | 26.039 | 1.00 | 17.71 | | N |
| ANISOU | 398 | ND2 | ASN | F | 82 | 2226 | 2562 | 1938 | 302 | −513 | −93 | N |
| ATOM | 401 | C | ASN | F | 82 | 2.657 | 60.972 | 23.832 | 1.00 | 16.29 | | C |
| ANISOU | 401 | C | ASN | F | 82 | 1919 | 2245 | 2024 | 181 | −59 | −10 | C |
| ATOM | 402 | O | ASN | F | 82 | 3.543 | 60.120 | 23.922 | 1.00 | 16.92 | | O |
| ANISOU | 402 | O | ASN | F | 82 | 1894 | 2273 | 2262 | 331 | −140 | 248 | O |
| ATOM | 404 | N | GLU | F | 83 | 1.482 | 60.748 | 23.254 | 1.00 | 15.38 | | N |
| ANISOU | 404 | N | GLU | F | 83 | 1768 | 2167 | 1908 | 166 | −117 | 103 | N |
| ATOM | 405 | CA | GLU | F | 83 | 1.122 | 59.460 | 22.708 | 1.00 | 15.56 | | C |
| ANISOU | 405 | CA | GLU | F | 83 | 1769 | 2174 | 1966 | 66 | −121 | 159 | C |
| ATOM | 407 | CB | GLU | F | 83 | 0.323 | 58.645 | 23.722 | 1.00 | 15.82 | | C |
| ANISOU | 407 | CB | GLU | F | 83 | 1762 | 2423 | 1824 | 11 | −26 | 114 | C |
| ATOM | 410 | CG | GLU | F | 83 | 0.182 | 57.173 | 23.361 | 1.00 | 17.01 | | C |
| ANISOU | 410 | CG | GLU | F | 83 | 2051 | 2399 | 2013 | 71 | 105 | 193 | C |
| ATOM | 413 | CD | GLU | F | 83 | 1.495 | 56.439 | 23.465 | 1.00 | 17.47 | | C |
| ANISOU | 413 | CD | GLU | F | 83 | 2138 | 2219 | 2277 | 143 | −323 | 82 | C |
| ATOM | 414 | OE1 | GLU | F | 83 | 2.253 | 56.725 | 24.420 | 1.00 | 18.03 | | O |
| ANISOU | 414 | OE1 | GLU | F | 83 | 2337 | 2471 | 2041 | 238 | −378 | −102 | O |
| ATOM | 415 | OE2 | GLU | F | 83 | 1.785 | 55.599 | 22.597 | 1.00 | 21.32 | | O |
| ANISOU | 415 | OE2 | GLU | F | 83 | 2568 | 2753 | 2778 | 419 | −326 | 36 | O |
| ATOM | 416 | C | GLU | F | 83 | 0.269 | 59.677 | 21.484 | 1.00 | 15.38 | | C |
| ANISOU | 416 | C | GLU | F | 83 | 1878 | 2137 | 1828 | 94 | −136 | 122 | C |
| ATOM | 417 | O | GLU | F | 83 | −0.584 | 60.562 | 21.469 | 1.00 | 16.73 | | O |
| ANISOU | 417 | O | GLU | F | 83 | 1909 | 2141 | 2305 | 138 | −208 | 141 | O |
| ATOM | 419 | N | TYR | F | 84 | 0.492 | 58.859 | 20.465 | 1.00 | 16.41 | | N |
| ANISOU | 419 | N | TYR | F | 84 | 1992 | 2357 | 1886 | 189 | −52 | 50 | N |
| ATOM | 420 | CA | TYR | F | 84 | −0.396 | 58.829 | 19.316 | 1.00 | 16.91 | | C |
| ANISOU | 420 | CA | TYR | F | 84 | 2060 | 2246 | 2116 | 27 | −164 | 28 | C |
| ATOM | 422 | CB | TYR | F | 84 | 0.291 | 58.152 | 18.129 | 1.00 | 23.05 | | C |
| ANISOU | 422 | CB | TYR | F | 84 | 2813 | 3058 | 2886 | 75 | 85 | −45 | C |
| ATOM | 425 | CG | TYR | F | 84 | −0.627 | 57.874 | 16.965 | 1.00 | 23.84 | | C |
| ANISOU | 425 | CG | TYR | F | 84 | 3023 | 3196 | 2837 | 34 | −37 | −50 | C |
| ATOM | 426 | CD1 | TYR | F | 84 | −1.450 | 58.867 | 16.445 | 1.00 | 27.48 | | C |
| ANISOU | 426 | CD1 | TYR | F | 84 | 3409 | 3684 | 3347 | 156 | −183 | −91 | C |
| ATOM | 428 | CE1 | TYR | F | 84 | −2.291 | 58.610 | 15.373 | 1.00 | 27.55 | | C |
| ANISOU | 428 | CE1 | TYR | F | 84 | 3358 | 3611 | 3499 | 100 | −175 | −223 | C |
| ATOM | 430 | CZ | TYR | F | 84 | −2.308 | 57.354 | 14.808 | 1.00 | 26.98 | | C |
| ANISOU | 430 | CZ | TYR | F | 84 | 3366 | 3519 | 3363 | 33 | −200 | −66 | C |
| ATOM | 431 | OH | TYR | F | 84 | −3.139 | 57.106 | 13.738 | 1.00 | 28.72 | | O |
| ANISOU | 431 | OH | TYR | F | 84 | 3429 | 3907 | 3574 | 39 | −251 | −342 | O |
| ATOM | 433 | CE2 | TYR | F | 84 | −1.494 | 56.356 | 15.300 | 1.00 | 26.53 | | C |
| ANISOU | 433 | CE2 | TYR | F | 84 | 3240 | 3581 | 3259 | 6 | −63 | −132 | C |
| ATOM | 435 | CD2 | TYR | F | 84 | −0.662 | 56.619 | 16.374 | 1.00 | 25.57 | | C |
| ANISOU | 435 | CD2 | TYR | F | 84 | 3263 | 3294 | 3157 | −7 | −101 | −66 | C |
| ATOM | 437 | C | TYR | F | 84 | −1.656 | 58.074 | 19.738 | 1.00 | 15.81 | | C |
| ANISOU | 437 | C | TYR | F | 84 | 1949 | 1984 | 2074 | 64 | −174 | 148 | C |
| ATOM | 438 | O | TYR | F | 84 | −1.608 | 56.865 | 19.994 | 1.00 | 17.73 | | O |
| ANISOU | 438 | O | TYR | F | 84 | 2004 | 2264 | 2466 | −68 | −63 | 359 | O |
| ATOM | 440 | N | GLN | F | 85 | −2.762 | 58.808 | 19.859 | 1.00 | 15.47 | | N |
| ANISOU | 440 | N | GLN | F | 85 | 1774 | 1942 | 2161 | −21 | −77 | 213 | N |
| ATOM | 441 | CA | GLN | F | 85 | −4.028 | 58.250 | 20.328 | 1.00 | 15.86 | | C |
| ANISOU | 441 | CA | GLN | F | 85 | 2106 | 2081 | 1836 | −6 | −99 | 167 | C |
| ATOM | 443 | CB | GLN | F | 85 | −4.837 | 59.281 | 21.134 | 1.00 | 15.93 | | C |
| ANISOU | 443 | CB | GLN | F | 85 | 2074 | 1871 | 2107 | 89 | −153 | 298 | C |
| ATOM | 446 | CG | GLN | F | 85 | −4.051 | 60.140 | 22.111 | 1.00 | 15.25 | | C |
| ANISOU | 446 | CG | GLN | F | 85 | 1960 | 2030 | 1801 | 110 | 65 | 134 | C |
| ATOM | 449 | CD | GLN | F | 85 | −3.591 | 59.417 | 23.358 | 1.00 | 15.31 | | C |
| ANISOU | 449 | CD | GLN | F | 85 | 1764 | 1913 | 2139 | 12 | −47 | 106 | C |
| ATOM | 450 | OE1 | GLN | F | 85 | −3.505 | 58.186 | 23.407 | 1.00 | 16.95 | | O |
| ANISOU | 450 | OE1 | GLN | F | 85 | 2286 | 2083 | 2068 | −90 | 125 | 169 | O |
| ATOM | 451 | NE2 | GLN | F | 85 | −3.265 | 60.195 | 24.379 | 1.00 | 17.03 | | N |
| ANISOU | 451 | NE2 | GLN | F | 85 | 1993 | 2400 | 2076 | −76 | −9 | −152 | N |
| ATOM | 454 | C | GLN | F | 85 | −4.845 | 57.810 | 19.131 | 1.00 | 17.47 | | C |
| ANISOU | 454 | C | GLN | F | 85 | 2162 | 2167 | 2309 | −5 | −256 | 65 | C |
| ATOM | 455 | O | GLN | F | 85 | −4.950 | 58.540 | 18.143 | 1.00 | 19.66 | | O |
| ANISOU | 455 | O | GLN | F | 85 | 2443 | 2432 | 2593 | −138 | −275 | 189 | O |
| ATOM | 457 | N | THR | F | 86 | −5.419 | 56.616 | 19.211 | 1.00 | 16.44 | | N |
| ANISOU | 457 | N | THR | F | 86 | 2000 | 2212 | 2030 | 79 | −137 | 114 | N |
| ATOM | 458 | CA | THR | F | 86 | −6.337 | 56.154 | 18.169 | 1.00 | 15.71 | | C |
| ANISOU | 458 | CA | THR | F | 86 | 1971 | 2054 | 1942 | −95 | −200 | 96 | C |
| ATOM | 460 | CB | THR | F | 86 | −5.783 | 54.921 | 17.406 | 1.00 | 16.05 | | C |
| ANISOU | 460 | CB | THR | F | 86 | 1867 | 2178 | 2052 | −29 | −91 | 117 | C |
| ATOM | 462 | OG1 | THR | F | 86 | −5.603 | 53.827 | 18.311 | 1.00 | 18.47 | | O |
| ANISOU | 462 | OG1 | THR | F | 86 | 2363 | 2318 | 2337 | 126 | −141 | 276 | O |
| ATOM | 464 | CG2 | THR | F | 86 | −4.446 | 55.264 | 16.738 | 1.00 | 18.52 | | C |
| ANISOU | 464 | CG2 | THR | F | 86 | 2213 | 2378 | 2445 | 122 | 110 | 80 | C |
| ATOM | 468 | C | THR | F | 86 | −7.744 | 55.882 | 18.709 | 1.00 | 17.57 | | C |
| ANISOU | 468 | C | THR | F | 86 | 2272 | 2184 | 2218 | −117 | 150 | 120 | C |
| ATOM | 469 | O | THR | F | 86 | −8.589 | 55.374 | 17.978 | 1.00 | 20.26 | | O |
| ANISOU | 469 | O | THR | F | 86 | 2405 | 2889 | 2401 | −372 | 25 | 1 | O |
| ATOM | 471 | N | MET | F | 87 | −7.986 | 56.209 | 19.980 | 1.00 | 18.29 | | N |
| ANISOU | 471 | N | MET | F | 87 | 2433 | 2204 | 2313 | −139 | −166 | −130 | N |
| ATOM | 472 | CA | MET | F | 87 | −9.334 | 56.211 | 20.557 | 1.00 | 18.67 | | C |
| ANISOU | 472 | CA | MET | F | 87 | 2318 | 2348 | 2427 | −105 | 7 | −41 | C |
| ATOM | 474 | CB | MET | F | 87 | −9.472 | 55.133 | 21.641 | 1.00 | 20.05 | | C |
| ANISOU | 474 | CB | MET | F | 87 | 2651 | 2550 | 2416 | −151 | 18 | −24 | C |
| ATOM | 477 | CG | MET | F | 87 | −9.790 | 53.746 | 21.076 | 1.00 | 22.62 | | C |
| ANISOU | 477 | CG | MET | F | 87 | 3160 | 2630 | 2805 | −39 | −21 | −62 | C |
| ATOM | 480 | SD | MET | F | 87 | −9.998 | 52.458 | 22.327 | 1.00 | 22.54 | | S |
| ANISOU | 480 | SD | MET | F | 87 | 2591 | 2900 | 3072 | −207 | −164 | −72 | S |
| ATOM | 481 | CE | MET | F | 87 | −8.344 | 52.498 | 22.842 | 1.00 | 13.96 | | C |
| ANISOU | 481 | CE | MET | F | 87 | 1483 | 1664 | 2154 | 65 | 89 | −285 | C |
| ATOM | 485 | C | MET | F | 87 | −9.654 | 57.583 | 21.151 | 1.00 | 20.47 | | C |
| ANISOU | 485 | C | MET | F | 87 | 2614 | 2627 | 2536 | −168 | 27 | −100 | C |
| ATOM | 486 | O | MET | F | 87 | −8.753 | 58.341 | 21.507 | 1.00 | 21.71 | | O |
| ANISOU | 486 | O | MET | F | 87 | 2614 | 2715 | 2917 | −288 | −223 | −253 | O |
| ATOM | 488 | N | GLU | F | 88 | −10.945 | 57.890 | 21.253 | 1.00 | 19.52 | | N |
| ANISOU | 488 | N | GLU | F | 88 | 2516 | 2492 | 2408 | −37 | 204 | −68 | N |
| ATOM | 489 | CA | GLU | F | 88 | −11.405 | 59.157 | 21.811 | 1.00 | 21.86 | | C |
| ANISOU | 489 | CA | GLU | F | 88 | 2867 | 2754 | 2683 | −54 | 157 | −61 | C |
| ATOM | 491 | CB | GLU | F | 88 | −12.669 | 59.621 | 21.094 | 1.00 | 24.93 | | C |
| ANISOU | 491 | CB | GLU | F | 88 | 3198 | 3254 | 3018 | 85 | 33 | 86 | C |
| ATOM | 494 | CG | GLU | F | 88 | −12.485 | 59.874 | 19.615 | 1.00 | 28.91 | | C |
| ANISOU | 494 | CG | GLU | F | 88 | 3786 | 3774 | 3423 | −39 | 13 | 18 | C |
| ATOM | 497 | CD | GLU | F | 88 | −13.706 | 60.513 | 18.993 | 1.00 | 31.58 | | C |
| ANISOU | 497 | CD | GLU | F | 88 | 3955 | 4099 | 3941 | 91 | −49 | 65 | C |
| ATOM | 498 | OE1 | GLU | F | 88 | −14.832 | 60.213 | 19.448 | 1.00 | 37.75 | | O |
| ANISOU | 498 | OE1 | GLU | F | 88 | 4454 | 4946 | 4942 | −20 | 184 | −25 | O |
| ATOM | 499 | OE2 | GLU | F | 88 | −13.543 | 61.314 | 18.048 | 1.00 | 36.75 | | O |
| ANISOU | 499 | OE2 | GLU | F | 88 | 4712 | 4622 | 4629 | −39 | 219 | 194 | O |
| ATOM | 500 | C | GLU | F | 88 | −11.726 | 59.044 | 23.292 | 1.00 | 18.59 | | C |
| ANISOU | 500 | C | GLU | F | 88 | 2460 | 2321 | 2281 | −72 | 140 | −33 | C |
| ATOM | 501 | O | GLU | F | 88 | −12.140 | 57.991 | 23.775 | 1.00 | 17.20 | | O |
| ANISOU | 501 | O | GLU | F | 88 | 2405 | 2141 | 1987 | −262 | −124 | −117 | O |
| ATOM | 503 | N | VAL | F | 89 | −11.543 | 60.149 | 24.005 | 1.00 | 19.38 | | N |
| ANISOU | 503 | N | VAL | F | 89 | 2513 | 2304 | 2545 | −113 | 126 | −83 | N |
| ATOM | 504 | CA | VAL | F | 89 | −12.087 | 60.314 | 25.344 | 1.00 | 20.00 | | C |
| ANISOU | 504 | CA | VAL | F | 89 | 2726 | 2343 | 2529 | −75 | 4 | 32 | C |
| ATOM | 506 | CB | VAL | F | 89 | −11.063 | 60.936 | 26.307 | 1.00 | 22.30 | | C |
| ANISOU | 506 | CB | VAL | F | 89 | 2833 | 2940 | 2698 | −151 | −21 | −105 | C |
| ATOM | 508 | CG1 | VAL | F | 89 | −11.701 | 61.204 | 27.673 | 1.00 | 24.53 | | C |
| ANISOU | 508 | CG1 | VAL | F | 89 | 3212 | 3157 | 2949 | −81 | 93 | −197 | C |
| ATOM | 512 | CG2 | VAL | F | 89 | −9.863 | 60.035 | 26.441 | 1.00 | 22.17 | | C |
| ANISOU | 512 | CG2 | VAL | F | 89 | 2965 | 2822 | 2635 | −98 | −201 | 8 | C |
| ATOM | 516 | C | VAL | F | 89 | −13.292 | 61.245 | 25.245 | 1.00 | 19.55 | | C |
| ANISOU | 516 | C | VAL | F | 89 | 2781 | 2366 | 2280 | 18 | −185 | 54 | C |
| ATOM | 517 | O | VAL | F | 89 | −13.207 | 62.333 | 24.661 | 1.00 | 22.14 | | O |
| ANISOU | 517 | O | VAL | F | 89 | 3262 | 2352 | 2796 | −17 | 272 | 179 | O |
| ATOM | 519 | N | GLN | F | 90 | −14.410 | 60.806 | 25.809 | 1.00 | 19.88 | | N |
| ANISOU | 519 | N | GLN | F | 90 | 2759 | 2193 | 2600 | 86 | −78 | −35 | N |
| ATOM | 520 | CA | GLN | F | 90 | −15.640 | 61.583 | 25.830 | 1.00 | 21.47 | | C |
| ANISOU | 520 | CA | GLN | F | 90 | 2758 | 2664 | 2736 | 91 | −7 | −8 | C |
| ATOM | 522 | CB | GLN | F | 90 | −16.658 | 60.993 | 24.840 | 1.00 | 22.51 | | C |
| ANISOU | 522 | CB | GLN | F | 90 | 2881 | 2821 | 2848 | 105 | −179 | 35 | C |
| ATOM | 525 | CG | GLN | F | 90 | −18.024 | 61.681 | 24.807 | 1.00 | 25.97 | | C |
| ANISOU | 525 | CG | GLN | F | 90 | 3188 | 3409 | 3270 | 21 | −43 | −3 | C |
| ATOM | 528 | CD | GLN | F | 90 | −19.057 | 60.928 | 23.964 | 1.00 | 28.52 | | C |
| ANISOU | 528 | CD | GLN | F | 90 | 3466 | 3654 | 3714 | −12 | −126 | −55 | C |
| ATOM | 529 | OE1 | GLN | F | 90 | −18.905 | 59.736 | 23.668 | 1.00 | 32.21 | | O |
| ANISOU | 529 | OE1 | GLN | F | 90 | 4140 | 3775 | 4322 | 136 | −69 | −140 | O |
| ATOM | 530 | NE2 | GLN | F | 90 | −20.122 | 61.625 | 23.584 | 1.00 | 32.15 | | N |
| ANISOU | 530 | NE2 | GLN | F | 90 | 3892 | 4274 | 4049 | 109 | −78 | 122 | N |
| ATOM | 533 | C | GLN | F | 90 | −16.182 | 61.545 | 27.253 | 1.00 | 18.25 | | C |
| ANISOU | 533 | C | GLN | F | 90 | 2270 | 2270 | 2391 | 19 | −254 | −103 | C |
| ATOM | 534 | O | GLN | F | 90 | −16.403 | 60.470 | 27.808 | 1.00 | 20.71 | | O |
| ANISOU | 534 | O | GLN | F | 90 | 2677 | 2473 | 2718 | −106 | −322 | −183 | O |
| ATOM | 536 | N | ASN | F | 91 | −16.380 | 62.720 | 27.845 | 1.00 | 21.03 | | N |
| ANISOU | 536 | N | ASN | F | 91 | 2701 | 2600 | 2689 | 163 | −227 | −5 | N |
| ATOM | 537 | CA | ASN | F | 91 | −16.901 | 62.822 | 29.201 | 1.00 | 21.23 | | C |
| ANISOU | 537 | CA | ASN | F | 91 | 2707 | 2676 | 2684 | 120 | −63 | −15 | C |
| ATOM | 539 | CB | ASN | F | 91 | −18.395 | 62.477 | 29.216 | 1.00 | 24.57 | | C |
| ANISOU | 539 | CB | ASN | F | 91 | 2982 | 3370 | 2984 | 17 | 15 | −30 | C |
| ATOM | 542 | CG | ASN | F | 91 | −19.132 | 63.080 | 30.409 | 1.00 | 25.14 | | C |
| ANISOU | 542 | CG | ASN | F | 91 | 3083 | 3293 | 3176 | 140 | 45 | −139 | C |
| ATOM | 543 | OD1 | ASN | F | 91 | −18.542 | 63.775 | 31.240 | 1.00 | 27.90 | | O |
| ANISOU | 543 | OD1 | ASN | F | 91 | 3371 | 3642 | 3586 | 341 | −68 | −261 | O |
| ATOM | 544 | ND2 | ASN | F | 91 | −20.437 | 62.821 | 30.483 | 1.00 | 28.13 | | N |
| ANISOU | 544 | ND2 | ASN | F | 91 | 3286 | 3837 | 3564 | 13 | 55 | −27 | N |
| ATOM | 547 | C | ASN | F | 91 | −16.132 | 61.924 | 30.169 | 1.00 | 17.84 | | C |
| ANISOU | 547 | C | ASN | F | 91 | 2336 | 2256 | 2186 | −79 | −3 | −189 | C |
| ATOM | 548 | O | ASN | F | 91 | −16.724 | 61.172 | 30.941 | 1.00 | 20.54 | | O |
| ANISOU | 548 | O | ASN | F | 91 | 2431 | 2792 | 2578 | −149 | 31 | −80 | O |
| ATOM | 550 | N | ASN | F | 92 | −14.803 | 61.994 | 30.091 | 1.00 | 16.95 | | N |
| ANISOU | 550 | N | ASN | F | 92 | 2254 | 1942 | 2244 | 61 | 109 | −10 | N |
| ATOM | 551 | CA | ASN | F | 92 | −13.917 | 61.260 | 30.994 | 1.00 | 16.49 | | C |
| ANISOU | 551 | CA | ASN | F | 92 | 2169 | 2043 | 2050 | 1 | 45 | −83 | C |
| ATOM | 553 | CB | ASN | F | 92 | −14.091 | 61.767 | 32.427 | 1.00 | 16.52 | | C |
| ANISOU | 553 | CB | ASN | F | 92 | 2177 | 1994 | 2105 | 73 | −19 | −74 | C |
| ATOM | 556 | CG | ASN | F | 92 | −13.666 | 63.204 | 32.565 | 1.00 | 16.31 | | C |
| ANISOU | 556 | CG | ASN | F | 92 | 2100 | 2034 | 2060 | −61 | −32 | −249 | C |
| ATOM | 557 | OD1 | ASN | F | 92 | −12.530 | 63.541 | 32.248 | 1.00 | 15.71 | | O |
| ANISOU | 557 | OD1 | ASN | F | 92 | 2113 | 1962 | 1893 | −248 | −117 | −166 | O |
| ATOM | 558 | ND2 | ASN | F | 92 | −14.584 | 64.070 | 33.001 | 1.00 | 18.63 | | N |
| ANISOU | 558 | ND2 | ASN | F | 92 | 2325 | 2284 | 2467 | 157 | 72 | −302 | N |
| ATOM | 561 | C | ASN | F | 92 | −14.051 | 59.736 | 30.915 | 1.00 | 16.96 | | C |
| ANISOU | 561 | C | ASN | F | 92 | 2471 | 1982 | 1991 | 43 | 44 | −157 | C |
| ATOM | 562 | O | ASN | F | 92 | −13.731 | 59.020 | 31.867 | 1.00 | 16.93 | | O |
| ANISOU | 562 | O | ASN | F | 92 | 2433 | 1937 | 2061 | −66 | −44 | −59 | O |
| ATOM | 564 | N | SER | F | 93 | −14.492 | 59.255 | 29.753 | 1.00 | 16.02 | | N |
| ANISOU | 564 | N | SER | F | 93 | 2430 | 1981 | 1673 | −129 | 31 | 4 | N |
| ATOM | 565 | CA | SER | F | 93 | −14.522 | 57.828 | 29.444 | 1.00 | 17.15 | | C |
| ANISOU | 565 | CA | SER | F | 93 | 2450 | 1941 | 2125 | 8 | 80 | −121 | C |
| ATOM | 567 | CB | ASER | F | 93 | −15.960 | 57.328 | 29.295 | 0.60 | 18.24 | | C |
| ANISOU | 567 | CB | ASER | F | 93 | 2542 | 2171 | 2217 | 81 | 121 | −117 | C |
| ATOM | 568 | CB | BSER | F | 93 | −15.959 | 57.320 | 29.349 | 0.40 | 18.45 | | C |
| ANISOU | 568 | CB | BSER | F | 93 | 2538 | 2132 | 2339 | −7 | 69 | −130 | C |
| ATOM | 573 | OG | ASER | F | 93 | −16.713 | 57.503 | 30.481 | 0.60 | 21.09 | | O |
| ANISOU | 573 | OG | ASER | F | 93 | 2523 | 3186 | 2302 | 195 | 329 | 313 | O |
| ATOM | 574 | OG | BSER | F | 93 | −16.589 | 57.777 | 28.167 | 0.40 | 20.44 | | O |
| ANISOU | 574 | OG | BSER | F | 93 | 2601 | 2582 | 2580 | −179 | −2 | 22 | O |
| ATOM | 577 | C | SER | F | 93 | −13.798 | 57.571 | 28.132 | 1.00 | 16.44 | | C |
| ANISOU | 577 | C | SER | F | 93 | 2313 | 1758 | 2172 | −111 | 40 | −99 | C |
| ATOM | 578 | O | SER | F | 93 | −13.916 | 58.351 | 27.182 | 1.00 | 17.56 | | O |
| ANISOU | 578 | O | SER | F | 93 | 2498 | 1968 | 2203 | 114 | 164 | −177 | O |
| ATOM | 580 | N | VAL | F | 94 | −13.091 | 56.448 | 28.067 | 1.00 | 14.99 | | N |
| ANISOU | 580 | N | VAL | F | 94 | 2056 | 1645 | 1993 | −160 | 10 | −40 | N |
| ATOM | 581 | CA | VAL | F | 94 | −12.469 | 56.017 | 26.827 | 1.00 | 15.91 | | C |
| ANISOU | 581 | CA | VAL | F | 94 | 2028 | 1953 | 2062 | −232 | 32 | −205 | C |
| ATOM | 583 | CB | VAL | F | 94 | −11.262 | 55.102 | 27.072 | 1.00 | 17.06 | | C |
| ANISOU | 583 | CB | VAL | F | 94 | 2166 | 2203 | 2112 | −153 | −41 | −121 | C |
| ATOM | 585 | CG1 | VAL | F | 94 | −10.612 | 54.710 | 25.754 | 1.00 | 17.02 | | C |
| ANISOU | 585 | CG1 | VAL | F | 94 | 2376 | 2055 | 2034 | −93 | −148 | −170 | C |
| ATOM | 589 | CG2 | VAL | F | 94 | −10.239 | 55.793 | 27.967 | 1.00 | 17.60 | | C |
| ANISOU | 589 | CG2 | VAL | F | 94 | 2096 | 2239 | 2352 | 11 | −58 | −239 | C |
| ATOM | 593 | C | VAL | F | 94 | −13.539 | 55.278 | 26.029 | 1.00 | 16.36 | | C |
| ANISOU | 593 | C | VAL | F | 94 | 2165 | 2098 | 1950 | −249 | −120 | −94 | C |
| ATOM | 594 | O | VAL | F | 94 | −14.149 | 54.330 | 26.527 | 1.00 | 15.62 | | O |
| ANISOU | 594 | O | VAL | F | 94 | 2223 | 1965 | 1747 | −276 | −85 | −170 | O |
| ATOM | 596 | N | VAL | F | 95 | −13.748 | 55.718 | 24.791 | 1.00 | 16.33 | | N |
| ANISOU | 596 | N | VAL | F | 95 | 2369 | 1956 | 1878 | −329 | −109 | −126 | N |
| ATOM | 597 | CA | VAL | F | 95 | −14.816 | 55.218 | 23.935 | 1.00 | 16.51 | | C |
| ANISOU | 597 | CA | VAL | F | 95 | 2102 | 2065 | 2106 | 61 | −154 | −32 | C |
| ATOM | 599 | CB | VAL | F | 95 | −15.352 | 56.361 | 23.035 | 1.00 | 17.65 | | C |
| ANISOU | 599 | CB | VAL | F | 95 | 2305 | 2180 | 2218 | 96 | −161 | −110 | C |
| ATOM | 601 | CG1 | VAL | F | 95 | −16.417 | 55.857 | 22.077 | 1.00 | 18.66 | | C |
| ANISOU | 601 | CG1 | VAL | F | 95 | 2410 | 2469 | 2210 | 2 | −191 | −121 | C |
| ATOM | 605 | CG2 | VAL | F | 95 | −15.870 | 57.502 | 23.901 | 1.00 | 18.00 | | C |
| ANISOU | 605 | CG2 | VAL | F | 95 | 2447 | 2025 | 2367 | −9 | −72 | −62 | C |
| ATOM | 609 | C | VAL | F | 95 | −14.293 | 54.088 | 23.062 | 1.00 | 15.55 | | C |
| ANISOU | 609 | C | VAL | F | 95 | 2053 | 1869 | 1985 | −156 | −165 | −66 | C |
| ATOM | 610 | O | VAL | F | 95 | −13.435 | 54.302 | 22.218 | 1.00 | 16.77 | | O |
| ANISOU | 610 | O | VAL | F | 95 | 2378 | 1847 | 2146 | −445 | 252 | −348 | O |
| ATOM | 612 | N | ILE | F | 96 | −14.809 | 52.881 | 23.264 | 1.00 | 14.81 | | N |
| ANISOU | 612 | N | ILE | F | 96 | 1952 | 1828 | 1845 | −73 | 227 | −59 | N |
| ATOM | 613 | CA | ILE | F | 96 | −14.377 | 51.738 | 22.468 | 1.00 | 13.75 | | C |
| ANISOU | 613 | CA | ILE | F | 96 | 1847 | 1715 | 1661 | −96 | −56 | 55 | C |
| ATOM | 615 | CB | ILE | F | 96 | −14.582 | 50.406 | 23.225 | 1.00 | 15.36 | | C |
| ANISOU | 615 | CB | ILE | F | 96 | 2036 | 1723 | 2075 | −87 | −43 | 60 | C |
| ATOM | 617 | CG1 | ILE | F | 96 | −13.895 | 50.448 | 24.599 | 1.00 | 15.78 | | C |
| ANISOU | 617 | CG1 | ILE | F | 96 | 1916 | 1970 | 2108 | 32 | −19 | 229 | C |
| ATOM | 620 | CD1 | ILE | F | 96 | −12.444 | 50.845 | 24.546 | 1.00 | 17.68 | | C |
| ANISOU | 620 | CD1 | ILE | F | 96 | 2286 | 2274 | 2157 | −165 | −93 | 0 | C |
| ATOM | 624 | CG2 | ILE | F | 96 | −14.074 | 49.224 | 22.388 | 1.00 | 14.61 | | C |
| ANISOU | 624 | CG2 | ILE | F | 96 | 1881 | 1746 | 1922 | −48 | 7 | −109 | C |
| ATOM | 628 | C | ILE | F | 96 | −15.133 | 51.713 | 21.146 | 1.00 | 13.88 | | C |
| ANISOU | 628 | C | ILE | F | 96 | 1748 | 1688 | 1837 | 58 | −71 | −69 | C |
| ATOM | 629 | O | ILE | F | 96 | −16.361 | 51.779 | 21.116 | 1.00 | 14.74 | | O |
| ANISOU | 629 | O | ILE | F | 96 | 1844 | 1854 | 1900 | 9 | 77 | 148 | O |
| ATOM | 631 | N | LYS | F | 97 | −14.384 | 51.615 | 20.052 | 1.00 | 15.75 | | N |
| ANISOU | 631 | N | LYS | F | 97 | 1996 | 1969 | 2019 | 39 | −6 | −55 | N |
| ATOM | 632 | CA | LYS | F | 97 | −14.975 | 51.525 | 18.721 | 1.00 | 17.05 | | C |
| ANISOU | 632 | CA | LYS | F | 97 | 2270 | 2133 | 2075 | 63 | 18 | −46 | C |
| ATOM | 634 | CB | LYS | F | 97 | −14.407 | 52.625 | 17.820 | 1.00 | 22.27 | | C |
| ANISOU | 634 | CB | LYS | F | 97 | 3079 | 2686 | 2696 | −63 | −28 | 151 | C |
| ATOM | 637 | CG | LYS | F | 97 | −14.872 | 54.028 | 18.198 | 1.00 | 24.55 | | C |
| ANISOU | 637 | CG | LYS | F | 97 | 3224 | 3124 | 2979 | 31 | 17 | −54 | C |
| ATOM | 640 | CD | LYS | F | 97 | −16.359 | 54.235 | 17.898 | 1.00 | 27.18 | | C |
| ANISOU | 640 | CD | LYS | F | 97 | 3459 | 3456 | 3410 | 107 | −17 | 39 | C |
| ATOM | 643 | CE | LYS | F | 97 | −16.651 | 55.611 | 17.308 | 1.00 | 28.29 | | C |
| ANISOU | 643 | CE | LYS | F | 97 | 3641 | 3385 | 3722 | 65 | 18 | 52 | C |
| ATOM | 646 | NZ | LYS | F | 97 | −17.668 | 55.520 | 16.220 | 1.00 | 30.77 | | N |
| ANISOU | 646 | NZ | LYS | F | 97 | 3987 | 3860 | 3843 | −107 | −62 | −1 | N |
| ATOM | 650 | C | LYS | F | 97 | −14.785 | 50.158 | 18.073 | 1.00 | 15.63 | | C |
| ANISOU | 650 | C | LYS | F | 97 | 1924 | 1975 | 2039 | −74 | −189 | −162 | C |
| ATOM | 651 | O | LYS | F | 97 | −15.608 | 49.755 | 17.258 | 1.00 | 16.53 | | O |
| ANISOU | 651 | O | LYS | F | 97 | 1886 | 2344 | 2048 | 102 | −110 | −255 | O |
| ATOM | 653 | N | CYS | F | 98 | −13.719 | 49.452 | 18.447 | 1.00 | 14.49 | | N |
| ANISOU | 653 | N | CYS | F | 98 | 1987 | 1772 | 1745 | −55 | −163 | −110 | N |
| ATOM | 654 | CA | CYS | F | 98 | −13.404 | 48.148 | 17.881 | 1.00 | 14.80 | | C |
| ANISOU | 654 | CA | CYS | F | 98 | 2097 | 1790 | 1735 | −105 | −129 | 18 | C |
| ATOM | 656 | CB | CYS | F | 98 | −12.044 | 48.175 | 17.191 | 1.00 | 15.14 | | C |
| ANISOU | 656 | CB | CYS | F | 98 | 2099 | 1864 | 1789 | −153 | −44 | −208 | C |
| ATOM | 659 | SG | CYS | F | 98 | −11.823 | 49.453 | 15.946 | 1.00 | 18.60 | | S |
| ANISOU | 659 | SG | CYS | F | 98 | 2388 | 2251 | 2428 | −324 | −20 | 100 | S |
| ATOM | 661 | C | CYS | F | 98 | −13.377 | 47.053 | 18.937 | 1.00 | 14.64 | | C |
| ANISOU | 661 | C | CYS | F | 98 | 1961 | 1815 | 1786 | 75 | −10 | −1 | C |
| ATOM | 662 | O | CYS | F | 98 | −12.811 | 47.221 | 20.012 | 1.00 | 15.41 | | O |
| ANISOU | 662 | O | CYS | F | 98 | 1894 | 2019 | 1940 | −69 | −316 | −91 | O |
| ATOM | 664 | N | ASP | F | 99 | −13.964 | 45.912 | 18.604 | 1.00 | 13.84 | | N |
| ANISOU | 664 | N | ASP | F | 99 | 1812 | 1665 | 1780 | −38 | −3 | 83 | N |
| ATOM | 665 | CA | ASP | F | 99 | −13.924 | 44.754 | 19.487 | 1.00 | 14.62 | | C |
| ANISOU | 665 | CA | ASP | F | 99 | 1909 | 1845 | 1801 | 104 | 78 | 123 | C |
| ATOM | 667 | CB | ASP | F | 99 | −14.752 | 43.601 | 18.924 | 1.00 | 16.18 | | C |
| ANISOU | 667 | CB | ASP | F | 99 | 1985 | 2031 | 2128 | −134 | −71 | 178 | C |
| ATOM | 670 | CG | ASP | F | 99 | −16.241 | 43.904 | 18.809 | 1.00 | 18.10 | | C |
| ANISOU | 670 | CG | ASP | F | 99 | 2294 | 2242 | 2341 | 96 | −159 | 88 | C |
| ATOM | 671 | OD1 | ASP | F | 99 | −16.714 | 45.016 | 19.147 | 1.00 | 19.14 | | O |
| ANISOU | 671 | OD1 | ASP | F | 99 | 2294 | 2629 | 2349 | 96 | −88 | 19 | O |
| ATOM | 672 | OD2 | ASP | F | 99 | −16.940 | 42.981 | 18.353 | 1.00 | 22.71 | | O |
| ANISOU | 672 | OD2 | ASP | F | 99 | 2685 | 2573 | 3370 | −268 | −34 | 33 | O |
| ATOM | 673 | C | ASP | F | 99 | −12.493 | 44.250 | 19.624 | 1.00 | 14.33 | | C |
| ANISOU | 673 | C | ASP | F | 99 | 1921 | 1758 | 1763 | 96 | 0 | 16 | C |
| ATOM | 674 | O | ASP | F | 99 | −11.685 | 44.360 | 18.704 | 1.00 | 14.84 | | O |
| ANISOU | 674 | O | ASP | F | 99 | 1722 | 1999 | 1916 | 47 | −8 | 334 | O |
| ATOM | 676 | N | GLY | F | 100 | −12.199 | 43.645 | 20.760 | 1.00 | 14.54 | | N |
| ANISOU | 676 | N | GLY | F | 100 | 1679 | 2016 | 1828 | 84 | 51 | 298 | N |
| ATOM | 677 | CA | GLY | F | 100 | −10.937 | 42.952 | 20.933 | 1.00 | 15.13 | | C |
| ANISOU | 677 | CA | GLY | F | 100 | 1885 | 1970 | 1894 | 0 | −81 | −69 | C |
| ATOM | 680 | C | GLY | F | 100 | −10.671 | 42.639 | 22.386 | 1.00 | 12.39 | | C |
| ANISOU | 680 | C | GLY | F | 100 | 1685 | 1606 | 1414 | −70 | 12 | 6 | C |
| ATOM | 681 | O | GLY | F | 100 | −11.386 | 43.120 | 23.289 | 1.00 | 14.68 | | O |
| ANISOU | 681 | O | GLY | F | 100 | 1689 | 1972 | 1916 | 118 | 98 | −14 | O |
| ATOM | 683 | N | LEU | F | 101 | −9.650 | 41.820 | 22.618 | 1.00 | 13.67 | | N |
| ANISOU | 683 | N | LEU | F | 101 | 1637 | 1873 | 1683 | 5 | 67 | −99 | N |
| ATOM | 684 | CA | LEU | F | 101 | −9.042 | 41.757 | 23.940 | 1.00 | 14.27 | | C |
| ANISOU | 684 | CA | LEU | F | 101 | 1788 | 1828 | 1803 | 82 | 28 | 20 | C |
| ATOM | 686 | CB | LEU | F | 101 | −8.216 | 40.492 | 24.125 | 1.00 | 14.74 | | C |
| ANISOU | 686 | CB | LEU | F | 101 | 1709 | 1969 | 1922 | 275 | 9 | 66 | C |
| ATOM | 689 | CG | LEU | F | 101 | −9.027 | 39.196 | 24.128 | 1.00 | 15.85 | | C |
| ANISOU | 689 | CG | LEU | F | 101 | 2027 | 2016 | 1980 | 91 | −36 | 188 | C |
| ATOM | 691 | CD1 | LEU | F | 101 | −8.103 | 37.990 | 24.102 | 1.00 | 18.28 | | C |
| ANISOU | 691 | CD1 | LEU | F | 101 | 2583 | 2219 | 2143 | 86 | 425 | −106 | C |
| ATOM | 695 | CD2 | LEU | F | 101 | −9.940 | 39.126 | 25.332 | 1.00 | 15.70 | | C |
| ANISOU | 695 | CD2 | LEU | F | 101 | 1981 | 1968 | 2014 | 43 | −16 | 102 | C |
| ATOM | 699 | C | LEU | F | 101 | −8.157 | 42.984 | 24.075 | 1.00 | 13.65 | | C |
| ANISOU | 699 | C | LEU | F | 101 | 1707 | 1668 | 1810 | 163 | −27 | −2 | C |
| ATOM | 700 | O | LEU | F | 101 | −7.434 | 43.331 | 23.136 | 1.00 | 15.01 | | O |
| ANISOU | 700 | O | LEU | F | 101 | 1809 | 2024 | 1870 | −77 | 112 | −11 | O |
| ATOM | 702 | N | TYR | F | 102 | −8.285 | 43.680 | 25.199 | 1.00 | 13.98 | | N |
| ANISOU | 702 | N | TYR | F | 102 | 1674 | 1806 | 1829 | 65 | 141 | −26 | N |
| ATOM | 703 | CA | TYR | F | 102 | −7.477 | 44.854 | 25.493 | 1.00 | 13.82 | | C |
| ANISOU | 703 | CA | TYR | F | 102 | 1666 | 1783 | 1802 | −42 | −14 | −114 | C |
| ATOM | 705 | CB | TYR | F | 102 | −8.351 | 46.093 | 25.606 | 1.00 | 14.12 | | C |
| ANISOU | 705 | CB | TYR | F | 102 | 1784 | 1789 | 1789 | −19 | 21 | 8 | C |
| ATOM | 708 | CG | TYR | F | 102 | −8.928 | 46.674 | 24.331 | 1.00 | 13.72 | | C |
| ANISOU | 708 | CG | TYR | F | 102 | 1686 | 1790 | 1737 | 93 | 69 | 23 | C |
| ATOM | 709 | CD1 | TYR | F | 102 | −9.950 | 46.035 | 23.638 | 1.00 | 13.32 | | C |
| ANISOU | 709 | CD1 | TYR | F | 102 | 1720 | 1636 | 1704 | −37 | 54 | 49 | C |
| ATOM | 711 | CE1 | TYR | F | 102 | −10.501 | 46.596 | 22.478 | 1.00 | 14.16 | | C |
| ANISOU | 711 | CE1 | TYR | F | 102 | 1746 | 1764 | 1867 | 6 | −204 | −70 | C |
| ATOM | 713 | CZ | TYR | F | 102 | −10.035 | 47.824 | 22.019 | 1.00 | 14.34 | | C |
| ANISOU | 713 | CZ | TYR | F | 102 | 1774 | 1847 | 1826 | 169 | 24 | 91 | C |
| ATOM | 714 | OH | TYR | F | 102 | −10.558 | 48.425 | 20.894 | 1.00 | 14.98 | | O |
| ANISOU | 714 | OH | TYR | F | 102 | 1942 | 1850 | 1899 | 77 | −23 | −59 | O |
| ATOM | 716 | CE2 | TYR | F | 102 | −9.040 | 48.479 | 22.708 | 1.00 | 14.53 | | C |
| ANISOU | 716 | CE2 | TYR | F | 102 | 1815 | 1879 | 1824 | −18 | 174 | 60 | C |
| ATOM | 718 | CD2 | TYR | F | 102 | −8.482 | 47.900 | 23.846 | 1.00 | 14.28 | | C |
| ANISOU | 718 | CD2 | TYR | F | 102 | 1721 | 1696 | 2009 | −33 | −22 | 66 | C |
| ATOM | 720 | C | TYR | F | 102 | −6.790 | 44.700 | 26.845 | 1.00 | 14.44 | | C |
| ANISOU | 720 | C | TYR | F | 102 | 1796 | 1810 | 1879 | 39 | 12 | −62 | C |
| ATOM | 721 | O | TYR | F | 102 | −7.348 | 44.110 | 27.776 | 1.00 | 15.36 | | O |
| ANISOU | 721 | O | TYR | F | 102 | 1894 | 2068 | 1872 | −91 | −168 | −18 | O |
| ATOM | 723 | N | ILE | F | 103 | −5.589 | 45.254 | 26.960 | 1.00 | 14.20 | | N |
| ANISOU | 723 | N | ILE | F | 103 | 1693 | 1908 | 1791 | −185 | −9 | −14 | N |
| ATOM | 724 | CA | ILE | F | 103 | −5.059 | 45.559 | 28.291 | 1.00 | 15.09 | | C |
| ANISOU | 724 | CA | ILE | F | 103 | 1841 | 1971 | 1921 | −143 | 74 | −102 | C |
| ATOM | 726 | CB | AILE | F | 103 | −3.530 | 45.684 | 28.256 | 0.65 | 16.66 | | C |
| ANISOU | 726 | CB | AILE | F | 103 | 1812 | 2514 | 2001 | −143 | 193 | −9 | C |
| ATOM | 727 | CB | BILE | F | 103 | −3.508 | 45.360 | 28.490 | 0.35 | 20.13 | | C |
| ANISOU | 727 | CB | BILE | F | 103 | 2308 | 2627 | 2714 | −158 | 120 | −46 | C |
| ATOM | 730 | CG1 | AILE | F | 103 | −2.894 | 44.334 | 27.998 | 0.65 | 16.80 | | C |
| ANISOU | 730 | CG1 | AILE | F | 103 | 2232 | 2201 | 1950 | −128 | 134 | −102 | C |
| ATOM | 731 | CG1 | BILE | F | 103 | −2.690 | 45.768 | 27.271 | 0.35 | 22.31 | | C |
| ANISOU | 731 | CG1 | BILE | F | 103 | 2726 | 3013 | 2738 | 25 | 53 | −59 | C |
| ATOM | 736 | CD1 | AILE | F | 103 | −1.391 | 44.385 | 27.825 | 0.65 | 16.49 | | C |
| ANISOU | 736 | CD1 | AILE | F | 103 | 2201 | 2119 | 1943 | −76 | −77 | −66 | C |
| ATOM | 737 | CD1 | BILE | F | 103 | −1.413 | 44.979 | 27.058 | 0.35 | 22.10 | | C |
| ANISOU | 737 | CD1 | BILE | F | 103 | 2616 | 2909 | 2870 | 9 | 111 | −20 | C |
| ATOM | 744 | CG2 | AILE | F | 103 | −2.994 | 46.303 | 29.568 | 0.65 | 17.36 | | C |
| ANISOU | 744 | CG2 | AILE | F | 103 | 1852 | 2710 | 2031 | −227 | −11 | −408 | C |
| ATOM | 745 | CG2 | BILE | F | 103 | −3.217 | 43.921 | 28.919 | 0.35 | 22.16 | | C |
| ANISOU | 745 | CG2 | BILE | F | 103 | 2610 | 2979 | 2830 | −6 | 14 | −20 | C |
| ATOM | 752 | C | ILE | F | 103 | −5.570 | 46.907 | 28.744 | 1.00 | 14.96 | | C |
| ANISOU | 752 | C | ILE | F | 103 | 1976 | 1823 | 1884 | −34 | −85 | −26 | C |
| ATOM | 753 | O | ILE | F | 103 | −5.661 | 47.845 | 27.956 | 1.00 | 16.30 | | O |
| ANISOU | 753 | O | ILE | F | 103 | 2584 | 1787 | 1822 | −214 | 46 | −57 | O |
| ATOM | 755 | N | ILE | F | 104 | −5.932 | 46.969 | 30.023 | 1.00 | 16.20 | | N |
| ANISOU | 755 | N | ILE | F | 104 | 2179 | 1947 | 2027 | 44 | 106 | 9 | N |
| ATOM | 756 | CA | ILE | F | 104 | −6.277 | 48.209 | 30.699 | 1.00 | 16.16 | | C |
| ANISOU | 756 | CA | ILE | F | 104 | 2362 | 1853 | 1925 | −94 | −71 | −94 | C |
| ATOM | 758 | CB | ILE | F | 104 | −7.738 | 48.245 | 31.198 | 1.00 | 16.78 | | C |
| ANISOU | 758 | CB | ILE | F | 104 | 2407 | 1962 | 2007 | −57 | −59 | 45 | C |
| ATOM | 760 | CG1 | ILE | F | 104 | −8.705 | 48.016 | 30.025 | 1.00 | 17.49 | | C |
| ANISOU | 760 | CG1 | ILE | F | 104 | 2344 | 2100 | 2200 | −43 | −140 | −84 | C |
| ATOM | 763 | CD1 | ILE | F | 104 | −10.147 | 47.800 | 30.441 | 1.00 | 18.65 | | C |
| ANISOU | 763 | CD1 | ILE | F | 104 | 2435 | 2157 | 2492 | −84 | −124 | −209 | C |
| ATOM | 767 | CG2 | ILE | F | 104 | −8.037 | 49.586 | 31.885 | 1.00 | 18.46 | | C |
| ANISOU | 767 | CG2 | ILE | F | 104 | 2622 | 2135 | 2254 | 16 | 59 | −10 | C |
| ATOM | 771 | C | ILE | F | 104 | −5.314 | 48.280 | 31.874 | 1.00 | 16.48 | | C |
| ANISOU | 771 | C | ILE | F | 104 | 2422 | 1921 | 1918 | −111 | −232 | −72 | C |
| ATOM | 772 | O | ILE | F | 104 | −5.273 | 47.374 | 32.704 | 1.00 | 18.15 | | O |
| ANISOU | 772 | O | ILE | F | 104 | 2912 | 2072 | 1911 | −226 | −177 | 87 | O |
| ATOM | 774 | N | TYR | F | 105 | −4.541 | 49.357 | 31.921 | 1.00 | 16.16 | | N |
| ANISOU | 774 | N | TYR | F | 105 | 2296 | 1835 | 2009 | −138 | −141 | −26 | N |
| ATOM | 775 | CA | TYR | F | 105 | −3.461 | 49.531 | 32.876 | 1.00 | 15.69 | | C |
| ANISOU | 775 | CA | TYR | F | 105 | 2187 | 1873 | 1900 | 14 | −73 | −34 | C |
| ATOM | 777 | CB | TYR | F | 105 | −2.135 | 49.460 | 32.147 | 1.00 | 17.36 | | C |
| ANISOU | 777 | CB | TYR | F | 105 | 2312 | 2170 | 2113 | 78 | −30 | −152 | C |
| ATOM | 780 | CG | TYR | F | 105 | −0.922 | 49.945 | 32.892 | 1.00 | 17.41 | | C |
| ANISOU | 780 | CG | TYR | F | 105 | 2344 | 2344 | 1926 | 146 | 137 | −232 | C |
| ATOM | 781 | CD1 | TYR | F | 105 | −0.224 | 49.104 | 33.741 | 1.00 | 17.51 | | C |
| ANISOU | 781 | CD1 | TYR | F | 105 | 2308 | 2323 | 2021 | 131 | 378 | −223 | C |
| ATOM | 783 | CE1 | TYR | F | 105 | 0.912 | 49.538 | 34.412 | 1.00 | 16.94 | | C |
| ANISOU | 783 | CE1 | TYR | F | 105 | 2033 | 2492 | 1908 | 131 | 369 | −62 | C |
| ATOM | 785 | CZ | TYR | F | 105 | 1.376 | 50.814 | 34.203 | 1.00 | 16.07 | | C |
| ANISOU | 785 | CZ | TYR | F | 105 | 2123 | 2185 | 1796 | 136 | 251 | −215 | C |
| ATOM | 786 | OH | TYR | F | 105 | 2.507 | 51.259 | 34.837 | 1.00 | 19.21 | | O |
| ANISOU | 786 | OH | TYR | F | 105 | 2224 | 2726 | 2349 | 145 | 160 | 45 | O |
| ATOM | 788 | CE2 | TYR | F | 105 | 0.692 | 51.675 | 33.355 | 1.00 | 17.52 | | C |
| ANISOU | 788 | CE2 | TYR | F | 105 | 2391 | 2142 | 2124 | 97 | −46 | −109 | C |
| ATOM | 790 | CD2 | TYR | F | 105 | −0.444 | 51.235 | 32.707 | 1.00 | 17.94 | | C |
| ANISOU | 790 | CD2 | TYR | F | 105 | 2291 | 2275 | 2250 | 86 | 192 | −280 | C |
| ATOM | 792 | C | TYR | F | 105 | −3.627 | 50.883 | 33.541 | 1.00 | 15.73 | | C |
| ANISOU | 792 | C | TYR | F | 105 | 2114 | 1863 | 1998 | −21 | 137 | −35 | C |
| ATOM | 793 | O | TYR | F | 105 | −3.825 | 51.891 | 32.863 | 1.00 | 15.44 | | O |
| ANISOU | 793 | O | TYR | F | 105 | 2219 | 1843 | 1803 | 103 | −1 | −10 | O |
| ATOM | 795 | N | LEU | F | 106 | −3.557 | 50.893 | 34.869 | 1.00 | 14.76 | | N |
| ANISOU | 795 | N | LEU | F | 106 | 2184 | 1700 | 1724 | −42 | −40 | −63 | N |
| ATOM | 796 | CA | LEU | F | 106 | −3.658 | 52.130 | 35.637 | 1.00 | 14.47 | | C |
| ANISOU | 796 | CA | LEU | F | 106 | 2007 | 1763 | 1727 | −21 | 30 | −65 | C |
| ATOM | 798 | CB | LEU | F | 106 | −4.920 | 52.141 | 36.507 | 1.00 | 15.78 | | C |
| ANISOU | 798 | CB | LEU | F | 106 | 2106 | 1947 | 1942 | −79 | 112 | 34 | C |
| ATOM | 801 | CG | LEU | F | 106 | −5.138 | 53.402 | 37.359 | 1.00 | 16.76 | | C |
| ANISOU | 801 | CG | LEU | F | 106 | 2018 | 2394 | 1956 | 52 | 81 | −109 | C |
| ATOM | 803 | CD1 | LEU | F | 106 | −5.375 | 54.626 | 36.499 | 1.00 | 18.79 | | C |
| ANISOU | 803 | CD1 | LEU | F | 106 | 2287 | 2381 | 2470 | 129 | 149 | −148 | C |
| ATOM | 807 | CD2 | LEU | F | 106 | −6.298 | 53.214 | 38.317 | 1.00 | 18.21 | | C |
| ANISOU | 807 | CD2 | LEU | F | 106 | 2094 | 2737 | 2087 | 115 | 149 | 144 | C |
| ATOM | 811 | C | LEU | F | 106 | −2.442 | 52.239 | 36.529 | 1.00 | 13.95 | | C |
| ANISOU | 811 | C | LEU | F | 106 | 1807 | 1826 | 1665 | 11 | 88 | −138 | C |
| ATOM | 812 | O | LEU | F | 106 | −2.086 | 51.284 | 37.214 | 1.00 | 14.71 | | O |
| ANISOU | 812 | O | LEU | F | 106 | 1858 | 1801 | 1929 | 176 | 1 | −63 | O |
| ATOM | 814 | N | LYS | F | 107 | −1.829 | 53.417 | 36.518 | 1.00 | 15.12 | | N |
| ANISOU | 814 | N | LYS | F | 107 | 1987 | 1830 | 1927 | 67 | 98 | −41 | N |
| ATOM | 815 | CA | LYS | F | 107 | −0.709 | 53.736 | 37.393 | 1.00 | 16.76 | | C |
| ANISOU | 815 | CA | LYS | F | 107 | 2285 | 1881 | 2201 | 129 | −144 | −206 | C |
| ATOM | 817 | CB | LYS | F | 107 | 0.600 | 53.741 | 36.583 | 1.00 | 22.43 | | C |
| ANISOU | 817 | CB | LYS | F | 107 | 2744 | 3101 | 2675 | 120 | 29 | −400 | C |
| ATOM | 820 | CG | LYS | F | 107 | 1.791 | 54.437 | 37.245 | 1.00 | 27.94 | | C |
| ANISOU | 820 | CG | LYS | F | 107 | 3176 | 3806 | 3633 | 45 | −32 | −218 | C |
| ATOM | 823 | CD | LYS | F | 107 | 3.025 | 54.394 | 36.361 | 1.00 | 29.70 | | C |
| ANISOU | 823 | CD | LYS | F | 107 | 3408 | 4042 | 3831 | −60 | 105 | −44 | C |
| ATOM | 826 | CE | LYS | F | 107 | 4.190 | 55.140 | 36.995 | 1.00 | 32.52 | | C |
| ANISOU | 826 | CE | LYS | F | 107 | 3751 | 4374 | 4228 | −162 | 66 | −14 | C |
| ATOM | 829 | NZ | LYS | F | 107 | 5.238 | 55.507 | 35.997 | 1.00 | 35.68 | | N |
| ANISOU | 829 | NZ | LYS | F | 107 | 4136 | 4786 | 4633 | −114 | 286 | 84 | N |
| ATOM | 833 | C | LYS | F | 107 | −0.951 | 55.106 | 38.002 | 1.00 | 15.25 | | C |
| ANISOU | 833 | C | LYS | F | 107 | 1937 | 1992 | 1863 | 25 | −49 | −84 | C |
| ATOM | 834 | O | LYS | F | 107 | −1.480 | 56.001 | 37.341 | 1.00 | 16.52 | | O |
| ANISOU | 834 | O | LYS | F | 107 | 2277 | 2046 | 1953 | 93 | −171 | −186 | O |
| ATOM | 836 | N | GLY | F | 108 | −0.551 | 55.275 | 39.254 | 1.00 | 15.84 | | N |
| ANISOU | 836 | N | GLY | F | 108 | 2138 | 1836 | 2044 | 205 | 26 | −188 | N |
| ATOM | 837 | CA | GLY | F | 108 | −0.525 | 56.604 | 39.835 | 1.00 | 17.05 | | C |
| ANISOU | 837 | CA | GLY | F | 108 | 2342 | 1989 | 2145 | 176 | −225 | −150 | C |
| ATOM | 840 | C | GLY | F | 108 | −0.120 | 56.573 | 41.279 | 1.00 | 15.99 | | C |
| ANISOU | 840 | C | GLY | F | 108 | 2090 | 1904 | 2080 | 96 | −283 | −48 | C |
| ATOM | 841 | O | GLY | F | 108 | −0.146 | 55.517 | 41.905 | 1.00 | 16.11 | | O |
| ANISOU | 841 | O | GLY | F | 108 | 2223 | 1925 | 1971 | 79 | 18 | −64 | O |
| ATOM | 843 | N | SER | F | 109 | 0.285 | 57.740 | 41.774 | 1.00 | 16.46 | | N |
| ANISOU | 843 | N | SER | F | 109 | 2193 | 1852 | 2208 | 100 | −67 | −76 | N |
| ATOM | 844 | CA | SER | F | 109 | 0.602 | 57.933 | 43.169 | 1.00 | 16.17 | | C |
| ANISOU | 844 | CA | SER | F | 109 | 2141 | 1917 | 2082 | 208 | 113 | −123 | C |
| ATOM | 846 | CB | SER | F | 109 | 1.926 | 58.678 | 43.335 | 1.00 | 19.13 | | C |
| ANISOU | 846 | CB | SER | F | 109 | 2283 | 2451 | 2532 | 14 | 76 | −89 | C |
| ATOM | 849 | OG | SER | F | 109 | 3.013 | 57.861 | 42.947 | 1.00 | 24.47 | | O |
| ANISOU | 849 | OG | SER | F | 109 | 2703 | 3217 | 3375 | 242 | 137 | 114 | O |
| ATOM | 851 | C | SER | F | 109 | −0.559 | 58.746 | 43.718 | 1.00 | 16.11 | | C |
| ANISOU | 851 | C | SER | F | 109 | 2078 | 1619 | 2422 | 154 | 208 | −164 | C |
| ATOM | 852 | O | SER | F | 109 | −0.820 | 59.859 | 43.274 | 1.00 | 17.46 | | O |
| ANISOU | 852 | O | SER | F | 109 | 2260 | 1772 | 2603 | 176 | 269 | 68 | O |
| ATOM | 854 | N | PHE | F | 110 | −1.306 | 58.128 | 44.615 | 1.00 | 19.35 | | N |
| ANISOU | 854 | N | PHE | F | 110 | 2598 | 2128 | 2624 | 294 | 602 | 61 | N |
| ATOM | 855 | CA | PHE | F | 110 | −2.567 | 58.672 | 45.091 | 1.00 | 20.30 | | C |
| ANISOU | 855 | CA | PHE | F | 110 | 2498 | 2378 | 2836 | 322 | 238 | 1 | C |
| ATOM | 857 | CB | PHE | F | 110 | −3.706 | 57.649 | 44.930 | 1.00 | 21.11 | | C |
| ANISOU | 857 | CB | PHE | F | 110 | 2688 | 2450 | 2880 | −36 | 193 | 19 | C |
| ATOM | 860 | CG | PHE | F | 110 | −4.078 | 57.364 | 43.495 | 1.00 | 23.82 | | C |
| ANISOU | 860 | CG | PHE | F | 110 | 2912 | 2961 | 3176 | −155 | 161 | 313 | C |
| ATOM | 861 | CD1 | PHE | F | 110 | −3.275 | 56.561 | 42.708 | 1.00 | 21.47 | | C |
| ANISOU | 861 | CD1 | PHE | F | 110 | 2723 | 2365 | 3066 | −196 | 0 | 42 | C |
| ATOM | 863 | CE1 | PHE | F | 110 | −3.599 | 56.310 | 41.385 | 1.00 | 23.75 | | C |
| ANISOU | 863 | CE1 | PHE | F | 110 | 2941 | 2822 | 3259 | −79 | −17 | 0 | C |
| ATOM | 865 | CZ | PHE | F | 110 | −4.759 | 56.839 | 40.843 | 1.00 | 23.58 | | C |
| ANISOU | 865 | CZ | PHE | F | 110 | 3021 | 2755 | 3181 | −45 | −27 | 6 | C |
| ATOM | 867 | CE2 | PHE | F | 110 | −5.579 | 57.625 | 41.624 | 1.00 | 23.62 | | C |
| ANISOU | 867 | CE2 | PHE | F | 110 | 2961 | 2895 | 3119 | −42 | 85 | 52 | C |
| ATOM | 869 | CD2 | PHE | F | 110 | −5.243 | 57.884 | 42.942 | 1.00 | 21.82 | | C |
| ANISOU | 869 | CD2 | PHE | F | 110 | 2720 | 2667 | 2901 | −195 | −59 | 9 | C |
| ATOM | 871 | C | PHE | F | 110 | −2.337 | 58.995 | 46.546 | 1.00 | 20.38 | | C |
| ANISOU | 871 | C | PHE | F | 110 | 2811 | 2258 | 2673 | 185 | 215 | −269 | C |
| ATOM | 872 | O | PHE | F | 110 | −1.917 | 58.137 | 47.310 | 1.00 | 24.51 | | O |
| ANISOU | 872 | O | PHE | F | 110 | 2919 | 3076 | 3316 | 673 | 369 | −90 | O |
| ATOM | 874 | N | PHE | F | 111 | −2.606 | 60.238 | 46.937 | 1.00 | 21.82 | | N |
| ANISOU | 874 | N | PHE | F | 111 | 2857 | 2287 | 3145 | 175 | 208 | −65 | N |
| ATOM | 875 | CA | PHE | F | 111 | −2.237 | 60.718 | 48.272 | 1.00 | 20.62 | | C |
| ANISOU | 875 | CA | PHE | F | 111 | 2578 | 2571 | 2682 | 10 | 100 | −25 | C |
| ATOM | 877 | CB | PHE | F | 111 | −1.686 | 62.150 | 48.182 | 1.00 | 20.86 | | C |
| ANISOU | 877 | CB | PHE | F | 111 | 2580 | 2522 | 2821 | 13 | 96 | 109 | C |
| ATOM | 880 | CG | PHE | F | 111 | −0.361 | 62.210 | 47.512 | 1.00 | 18.38 | | C |
| ANISOU | 880 | CG | PHE | F | 111 | 2274 | 1883 | 2824 | −71 | −12 | −63 | C |
| ATOM | 881 | CD1 | PHE | F | 111 | −0.271 | 62.102 | 46.131 | 1.00 | 20.61 | | C |
| ANISOU | 881 | CD1 | PHE | F | 111 | 2345 | 2618 | 2866 | −140 | 21 | 188 | C |
| ATOM | 883 | CE1 | PHE | F | 111 | 0.954 | 62.104 | 45.500 | 1.00 | 21.81 | | C |
| ANISOU | 883 | CE1 | PHE | F | 111 | 2716 | 2883 | 2687 | −94 | 94 | 188 | C |
| ATOM | 885 | CZ | PHE | F | 111 | 2.112 | 62.194 | 46.249 | 1.00 | 20.99 | | C |
| ANISOU | 885 | CZ | PHE | F | 111 | 2527 | 2637 | 2809 | −166 | 2 | 142 | C |
| ATOM | 887 | CE2 | PHE | F | 111 | 2.036 | 62.280 | 47.629 | 1.00 | 20.15 | | C |
| ANISOU | 887 | CE2 | PHE | F | 111 | 2513 | 2501 | 2639 | 89 | 73 | 267 | C |
| ATOM | 889 | CD2 | PHE | F | 111 | 0.805 | 62.278 | 48.256 | 1.00 | 18.82 | | C |
| ANISOU | 889 | CD2 | PHE | F | 111 | 2432 | 2176 | 2542 | −161 | −141 | 56 | C |
| ATOM | 891 | C | PHE | F | 111 | −3.350 | 60.583 | 49.318 | 1.00 | 21.22 | | C |
| ANISOU | 891 | C | PHE | F | 111 | 2654 | 2449 | 2960 | −3 | 228 | 19 | C |
| ATOM | 892 | O | PHE | F | 111 | −3.267 | 61.148 | 50.405 | 1.00 | 19.63 | | O |
| ANISOU | 892 | O | PHE | F | 111 | 2495 | 2107 | 2854 | 170 | 331 | 129 | O |
| ATOM | 894 | N | GLN | F | 112 | −4.389 | 59.829 | 48.972 | 1.00 | 21.77 | | N |
| ANISOU | 894 | N | GLN | F | 112 | 2871 | 2548 | 2850 | 21 | 190 | 31 | N |
| ATOM | 895 | CA | GLN | F | 112 | −5.268 | 59.221 | 49.968 | 1.00 | 23.59 | | C |
| ANISOU | 895 | CA | GLN | F | 112 | 2986 | 2807 | 3171 | −118 | 157 | 18 | C |
| ATOM | 897 | CB | GLN | F | 112 | −6.672 | 59.816 | 49.903 | 1.00 | 24.19 | | C |
| ANISOU | 897 | CB | GLN | F | 112 | 2973 | 3039 | 3177 | −96 | 22 | 29 | C |
| ATOM | 900 | CG | GLN | F | 112 | −6.737 | 61.346 | 49.907 | 1.00 | 23.90 | | C |
| ANISOU | 900 | CG | GLN | F | 112 | 2972 | 2968 | 3140 | 27 | 29 | 83 | C |
| ATOM | 903 | CD | GLN | F | 112 | −8.088 | 61.879 | 49.431 | 1.00 | 24.82 | | C |
| ANISOU | 903 | CD | GLN | F | 112 | 3013 | 3133 | 3284 | 22 | 4 | 4 | C |
| ATOM | 904 | OE1 | GLN | F | 112 | −8.906 | 61.130 | 48.888 | 1.00 | 25.85 | | O |
| ANISOU | 904 | OE1 | GLN | F | 112 | 2917 | 3242 | 3660 | 224 | 76 | 41 | O |
| ATOM | 905 | NE2 | GLN | F | 112 | −8.321 | 63.180 | 49.625 | 1.00 | 22.82 | | N |
| ANISOU | 905 | NE2 | GLN | F | 112 | 2520 | 2974 | 3176 | 126 | 198 | 27 | N |
| ATOM | 908 | C | GLN | F | 112 | −5.323 | 57.731 | 49.638 | 1.00 | 24.59 | | C |
| ANISOU | 908 | C | GLN | F | 112 | 3230 | 3000 | 3110 | −180 | −30 | −143 | C |
| ATOM | 909 | O | GLN | F | 112 | −5.178 | 57.359 | 48.478 | 1.00 | 25.87 | | O |
| ANISOU | 909 | O | GLN | F | 112 | 3487 | 2687 | 3655 | −427 | 486 | −360 | O |
| ATOM | 911 | N | GLU | F | 113 | −5.491 | 56.887 | 50.654 | 1.00 | 25.73 | | N |
| ANISOU | 911 | N | GLU | F | 113 | 3346 | 2993 | 3437 | −262 | 64 | −136 | N |
| ATOM | 912 | CA | GLU | F | 113 | −5.797 | 55.481 | 50.417 | 1.00 | 24.69 | | C |
| ANISOU | 912 | CA | GLU | F | 113 | 3077 | 3072 | 3231 | −44 | 69 | −61 | C |
| ATOM | 914 | CB | GLU | F | 113 | −5.799 | 54.688 | 51.719 | 1.00 | 27.35 | | C |
| ANISOU | 914 | CB | GLU | F | 113 | 3470 | 3414 | 3506 | 61 | 61 | 39 | C |
| ATOM | 917 | CG | GLU | F | 113 | −5.972 | 53.190 | 51.517 | 1.00 | 30.11 | | C |
| ANISOU | 917 | CG | GLU | F | 113 | 3849 | 3638 | 3951 | −60 | 60 | 28 | C |
| ATOM | 920 | CD | GLU | F | 113 | −4.943 | 52.370 | 52.277 | 1.00 | 34.01 | | C |
| ANISOU | 920 | CD | GLU | F | 113 | 4259 | 4325 | 4338 | 70 | −46 | 92 | C |
| ATOM | 921 | OE1 | GLU | F | 113 | −4.770 | 52.599 | 53.493 | 1.00 | 36.98 | | O |
| ANISOU | 921 | OE1 | GLU | F | 113 | 4650 | 4858 | 4540 | 40 | 55 | 28 | O |
| ATOM | 922 | OE2 | GLU | F | 113 | −4.306 | 51.494 | 51.651 | 1.00 | 38.16 | | O |
| ANISOU | 922 | OE2 | GLU | F | 113 | 4708 | 4736 | 5055 | 92 | 57 | 24 | O |
| ATOM | 923 | C | GLU | F | 113 | −7.163 | 55.469 | 49.762 | 1.00 | 22.34 | | C |
| ANISOU | 923 | C | GLU | F | 113 | 2979 | 2933 | 2576 | 14 | 55 | 1 | C |
| ATOM | 924 | O | GLU | F | 113 | −8.122 | 56.003 | 50.317 | 1.00 | 23.49 | | O |
| ANISOU | 924 | O | GLU | F | 113 | 2809 | 2973 | 3141 | 28 | −170 | −196 | O |
| ATOM | 926 | N | VAL | F | 114 | −7.239 | 54.891 | 48.567 | 1.00 | 21.67 | | N |
| ANISOU | 926 | N | VAL | F | 114 | 2753 | 2686 | 2792 | −2 | −45 | −73 | N |
| ATOM | 927 | CA | VAL | F | 114 | −8.395 | 55.079 | 47.695 | 1.00 | 22.38 | | C |
| ANISOU | 927 | CA | VAL | F | 114 | 2752 | 2747 | 3004 | −28 | 10 | 0 | C |
| ATOM | 929 | CB | VAL | F | 114 | −8.045 | 55.883 | 46.403 | 1.00 | 22.75 | | C |
| ANISOU | 929 | CB | VAL | F | 114 | 2860 | 2826 | 2956 | −53 | −58 | −29 | C |
| ATOM | 931 | CG1 | VAL | F | 114 | −7.823 | 57.350 | 46.731 | 1.00 | 25.86 | | C |
| ANISOU | 931 | CG1 | VAL | F | 114 | 3385 | 3036 | 3401 | 18 | 43 | 55 | C |
| ATOM | 935 | CG2 | VAL | F | 114 | −6.832 | 55.295 | 45.684 | 1.00 | 23.38 | | C |
| ANISOU | 935 | CG2 | VAL | F | 114 | 2938 | 2944 | 2998 | −53 | 61 | 107 | C |
| ATOM | 939 | C | VAL | F | 114 | −9.012 | 53.758 | 47.298 | 1.00 | 21.58 | | C |
| ANISOU | 939 | C | VAL | F | 114 | 2688 | 2588 | 2922 | 5 | −1 | −118 | C |
| ATOM | 940 | O | VAL | F | 114 | −8.313 | 52.746 | 47.153 | 1.00 | 22.59 | | O |
| ANISOU | 940 | O | VAL | F | 114 | 2493 | 2867 | 3219 | 33 | −66 | −358 | O |
| ATOM | 942 | N | LYS | F | 115 | −10.332 | 53.782 | 47.142 | 1.00 | 21.32 | | N |
| ANISOU | 942 | N | LYS | F | 115 | 2650 | 2594 | 2855 | 15 | −198 | 76 | N |
| ATOM | 943 | CA | LYS | F | 115 | −11.089 | 52.668 | 46.592 | 1.00 | 20.88 | | C |
| ANISOU | 943 | CA | LYS | F | 115 | 2716 | 2659 | 2558 | −13 | −175 | 32 | C |
| ATOM | 945 | CB | LYS | F | 115 | −12.249 | 52.295 | 47.517 | 1.00 | 23.20 | | C |
| ANISOU | 945 | CB | LYS | F | 115 | 2874 | 2982 | 2959 | −41 | −54 | −21 | C |
| ATOM | 948 | CG | LYS | F | 115 | −11.598 | 51.652 | 48.816 | 0.00 | 30.00 | | C |
| ANISOU | 948 | CG | LYS | F | 115 | 3799 | 3799 | 3799 | 0 | 0 | 0 | C |
| ATOM | 951 | CD | LYS | F | 115 | −11.630 | 50.122 | 48.747 | 0.00 | 30.00 | | C |
| ANISOU | 951 | CD | LYS | F | 115 | 3799 | 3799 | 3799 | 0 | 0 | 0 | C |
| ATOM | 954 | CE | LYS | F | 115 | −11.035 | 49.514 | 50.021 | 0.00 | 30.00 | | C |
| ANISOU | 954 | CE | LYS | F | 115 | 3799 | 3799 | 3799 | 0 | 0 | 0 | C |
| ATOM | 957 | NZ | LYS | F | 115 | −11.067 | 48.012 | 49.953 | 0.00 | 30.00 | | N |
| ANISOU | 957 | NZ | LYS | F | 115 | 3799 | 3799 | 3799 | 0 | 0 | 0 | N |
| ATOM | 961 | C | LYS | F | 115 | −11.607 | 53.108 | 45.229 | 1.00 | 20.11 | | C |
| ANISOU | 961 | C | LYS | F | 115 | 2481 | 2559 | 2598 | −94 | −61 | 189 | C |
| ATOM | 962 | O | LYS | F | 115 | −12.642 | 53.778 | 45.141 | 1.00 | 19.68 | | O |
| ANISOU | 962 | O | LYS | F | 115 | 2584 | 2925 | 1968 | 23 | −78 | 367 | O |
| ATOM | 964 | N | ILE | F | 116 | −10.860 | 52.753 | 44.186 | 1.00 | 18.52 | | N |
| ANISOU | 964 | N | ILE | F | 116 | 2327 | 2320 | 2389 | −170 | −361 | −63 | N |
| ATOM | 965 | CA | ILE | F | 116 | −11.212 | 53.095 | 42.810 | 1.00 | 18.46 | | C |
| ANISOU | 965 | CA | ILE | F | 116 | 2291 | 2407 | 2312 | −29 | −18 | −8 | C |
| ATOM | 967 | CB | ILE | F | 116 | −9.973 | 53.528 | 41.997 | 1.00 | 20.49 | | C |
| ANISOU | 967 | CB | ILE | F | 116 | 2485 | 2595 | 2703 | −34 | 84 | 136 | C |
| ATOM | 969 | CG1 | ILE | F | 116 | −9.371 | 54.798 | 42.603 | 1.00 | 22.40 | | C |
| ANISOU | 969 | CG1 | ILE | F | 116 | 2575 | 2872 | 3062 | −87 | −3 | 68 | C |
| ATOM | 972 | CD1 | ILE | F | 116 | −8.009 | 55.162 | 42.049 | 1.00 | 23.39 | | C |
| ANISOU | 972 | CD1 | ILE | F | 116 | 2615 | 3043 | 3226 | −114 | −95 | 162 | C |
| ATOM | 976 | CG2 | ILE | F | 116 | −10.335 | 53.769 | 40.537 | 1.00 | 21.13 | | C |
| ANISOU | 976 | CG2 | ILE | F | 116 | 2523 | 2673 | 2829 | −99 | −41 | 78 | C |
| ATOM | 980 | C | ILE | F | 116 | −11.834 | 51.879 | 42.131 | 1.00 | 17.28 | | C |
| ANISOU | 980 | C | ILE | F | 116 | 2137 | 2099 | 2326 | 6 | −129 | −37 | C |
| ATOM | 981 | O | ILE | F | 116 | −11.321 | 50.767 | 42.257 | 1.00 | 19.11 | | O |
| ANISOU | 981 | O | ILE | F | 116 | 2409 | 2049 | 2802 | −88 | −241 | −74 | O |
| ATOM | 983 | N | ASP | F | 117 | −12.923 | 52.108 | 41.397 | 1.00 | 15.31 | | N |
| ANISOU | 983 | N | ASP | F | 117 | 1992 | 1784 | 2040 | 29 | −145 | 75 | N |
| ATOM | 984 | CA | ASP | F | 117 | −13.569 | 51.077 | 40.589 | 1.00 | 15.89 | | C |
| ANISOU | 984 | CA | ASP | F | 117 | 2136 | 1999 | 1902 | −20 | −90 | 13 | C |
| ATOM | 986 | CB | ASP | F | 117 | −15.079 | 51.070 | 40.835 | 1.00 | 17.20 | | C |
| ANISOU | 986 | CB | ASP | F | 117 | 2144 | 2302 | 2086 | −79 | −46 | −35 | C |
| ATOM | 989 | CG | ASP | F | 117 | −15.482 | 50.312 | 42.095 | 1.00 | 21.32 | | C |
| ANISOU | 989 | CG | ASP | F | 117 | 2466 | 3081 | 2553 | 40 | 152 | 256 | C |
| ATOM | 990 | OD1 | ASP | F | 117 | −14.647 | 49.603 | 42.687 | 1.00 | 23.05 | | O |
| ANISOU | 990 | OD1 | ASP | F | 117 | 2763 | 3069 | 2924 | 402 | 437 | 273 | O |
| ATOM | 991 | OD2 | ASP | F | 117 | −16.663 | 50.425 | 42.498 | 1.00 | 27.77 | | O |
| ANISOU | 991 | OD2 | ASP | F | 117 | 3006 | 4046 | 3499 | 180 | 230 | 362 | O |
| ATOM | 992 | C | ASP | F | 117 | −13.319 | 51.339 | 39.109 | 1.00 | 14.80 | | C |
| ANISOU | 992 | C | ASP | F | 117 | 1929 | 1753 | 1939 | 14 | −135 | 31 | C |
| ATOM | 993 | O | ASP | F | 117 | −13.548 | 52.452 | 38.622 | 1.00 | 15.53 | | O |
| ANISOU | 993 | O | ASP | F | 117 | 2138 | 1675 | 2087 | 101 | −165 | −131 | O |
| ATOM | 995 | N | LEU | F | 118 | −12.862 | 50.303 | 38.407 | 1.00 | 15.66 | | N |
| ANISOU | 995 | N | LEU | F | 118 | 2097 | 1943 | 1909 | −5 | −126 | −167 | N |
| ATOM | 996 | CA | LEU | F | 118 | −12.700 | 50.326 | 36.956 | 1.00 | 15.55 | | C |
| ANISOU | 996 | CA | LEU | F | 118 | 1987 | 2109 | 1811 | −113 | −106 | −127 | C |
| ATOM | 998 | CB | LEU | F | 118 | −11.482 | 49.498 | 36.529 | 1.00 | 16.89 | | C |
| ANISOU | 998 | CB | LEU | F | 118 | 2118 | 2084 | 2215 | −111 | −218 | −72 | C |
| ATOM | 1001 | CG | LEU | F | 118 | −11.319 | 49.235 | 35.026 | 1.00 | 19.16 | | C |
| ANISOU | 1001 | CG | LEU | F | 118 | 2518 | 2323 | 2435 | 92 | −52 | −212 | C |
| ATOM | 1003 | CD1 | LEU | F | 118 | −11.073 | 50.519 | 34.280 | 1.00 | 19.19 | | C |
| ANISOU | 1003 | CD1 | LEU | F | 118 | 2715 | 2304 | 2270 | 158 | −99 | −252 | C |
| ATOM | 1007 | CD2 | LEU | F | 118 | −10.201 | 48.240 | 34.755 | 1.00 | 19.16 | | C |
| ANISOU | 1007 | CD2 | LEU | F | 118 | 2220 | 2707 | 2353 | 211 | −10 | −60 | C |
| ATOM | 1011 | C | LEU | F | 118 | −13.951 | 49.757 | 36.283 | 1.00 | 15.63 | | C |
| ANISOU | 1011 | C | LEU | F | 118 | 1968 | 1960 | 2011 | −120 | −49 | −113 | C |
| ATOM | 1012 | O | LEU | F | 118 | −14.297 | 48.588 | 36.479 | 1.00 | 16.62 | | O |
| ANISOU | 1012 | O | LEU | F | 118 | 2220 | 1845 | 2246 | −232 | −181 | 232 | O |
| ATOM | 1014 | N | HIS | F | 119 | −14.616 | 50.607 | 35.508 | 1.00 | 15.26 | | N |
| ANISOU | 1014 | N | HIS | F | 119 | 2024 | 1826 | 1947 | −160 | −52 | −164 | N |
| ATOM | 1015 | CA | HIS | F | 119 | −15.784 | 50.241 | 34.718 | 1.00 | 16.07 | | C |
| ANISOU | 1015 | CA | HIS | F | 119 | 2081 | 1929 | 2095 | −81 | −61 | −156 | C |
| ATOM | 1017 | CB | HIS | F | 119 | −16.842 | 51.344 | 34.790 | 1.00 | 16.77 | | C |
| ANISOU | 1017 | CB | HIS | F | 119 | 2040 | 2250 | 2080 | −77 | 3 | 0 | C |
| ATOM | 1020 | CG | HIS | F | 119 | −17.264 | 51.694 | 36.183 | 1.00 | 16.91 | | C |
| ANISOU | 1020 | CG | HIS | F | 119 | 2273 | 2108 | 2043 | −105 | 3 | 28 | C |
| ATOM | 1021 | ND1 | HIS | F | 119 | −18.506 | 51.376 | 36.686 | 1.00 | 21.55 | | N |
| ANISOU | 1021 | ND1 | HIS | F | 119 | 2668 | 2928 | 2592 | 102 | −23 | 2 | N |
| ATOM | 1023 | CE1 | HIS | F | 119 | −18.603 | 51.827 | 37.926 | 1.00 | 21.15 | | C |
| ANISOU | 1023 | CE1 | HIS | F | 119 | 2528 | 3078 | 2429 | −31 | 276 | −46 | C |
| ATOM | 1025 | NE2 | HIS | F | 119 | −17.471 | 52.429 | 38.242 | 1.00 | 20.97 | | N |
| ANISOU | 1025 | NE2 | HIS | F | 119 | 2593 | 3000 | 2372 | 209 | 138 | −123 | N |
| ATOM | 1027 | CD2 | HIS | F | 119 | −16.614 | 52.356 | 37.170 | 1.00 | 18.10 | | C |
| ANISOU | 1027 | CD2 | HIS | F | 119 | 2158 | 2478 | 2242 | 3 | 13 | −108 | C |
| ATOM | 1029 | C | HIS | F | 119 | −15.335 | 50.070 | 33.276 | 1.00 | 17.52 | | C |
| ANISOU | 1029 | C | HIS | F | 119 | 2346 | 2075 | 2236 | −184 | 86 | −131 | C |
| ATOM | 1030 | O | HIS | F | 119 | −14.670 | 50.942 | 32.724 | 1.00 | 18.05 | | O |
| ANISOU | 1030 | O | HIS | F | 119 | 2689 | 2148 | 2020 | −327 | −50 | −196 | O |
| ATOM | 1032 | N | PHE | F | 120 | −15.694 | 48.943 | 32.670 | 1.00 | 17.51 | | N |
| ANISOU | 1032 | N | PHE | F | 120 | 2367 | 2155 | 2129 | −341 | −152 | −127 | N |
| ATOM | 1033 | CA | PHE | F | 120 | −15.238 | 48.637 | 31.315 | 1.00 | 19.20 | | C |
| ANISOU | 1033 | CA | PHE | F | 120 | 2464 | 2367 | 2462 | −184 | 23 | −217 | C |
| ATOM | 1035 | CB | PHE | F | 120 | −14.000 | 47.720 | 31.354 | 1.00 | 21.14 | | C |
| ANISOU | 1035 | CB | PHE | F | 120 | 2663 | 2786 | 2582 | 50 | 1 | −352 | C |
| ATOM | 1038 | CG | PHE | F | 120 | −14.225 | 46.397 | 32.044 | 1.00 | 21.25 | | C |
| ANISOU | 1038 | CG | PHE | F | 120 | 2765 | 2764 | 2543 | 251 | −76 | −256 | C |
| ATOM | 1039 | CD1 | PHE | F | 120 | −14.313 | 46.315 | 33.431 | 1.00 | 25.44 | | C |
| ANISOU | 1039 | CD1 | PHE | F | 120 | 3731 | 2975 | 2958 | 127 | 54 | −59 | C |
| ATOM | 1041 | CE1 | PHE | F | 120 | −14.516 | 45.093 | 34.067 | 1.00 | 26.15 | | C |
| ANISOU | 1041 | CE1 | PHE | F | 120 | 3690 | 3282 | 2963 | 138 | −36 | −53 | C |
| ATOM | 1043 | CZ | PHE | F | 120 | −14.620 | 43.935 | 33.321 | 1.00 | 24.99 | | C |
| ANISOU | 1043 | CZ | PHE | F | 120 | 3397 | 3010 | 3088 | 57 | −14 | 49 | C |
| ATOM | 1045 | CE2 | PHE | F | 120 | −14.517 | 43.999 | 31.940 | 1.00 | 24.52 | | C |
| ANISOU | 1045 | CE2 | PHE | F | 120 | 3259 | 3027 | 3028 | 185 | −70 | −138 | C |
| ATOM | 1047 | CD2 | PHE | F | 120 | −14.313 | 45.222 | 31.310 | 1.00 | 21.18 | | C |
| ANISOU | 1047 | CD2 | PHE | F | 120 | 2475 | 2657 | 2912 | −108 | 137 | −348 | C |
| ATOM | 1049 | C | PHE | F | 120 | −16.336 | 48.075 | 30.408 | 1.00 | 18.39 | | C |
| ANISOU | 1049 | C | PHE | F | 120 | 2541 | 1987 | 2459 | −113 | 66 | −161 | C |
| ATOM | 1050 | O | PHE | F | 120 | −16.254 | 48.192 | 29.187 | 1.00 | 19.63 | | O |
| ANISOU | 1050 | O | PHE | F | 120 | 3020 | 2039 | 2400 | −209 | −51 | −333 | O |
| ATOM | 1052 | N | ARG | F | 121 | −17.368 | 47.483 | 30.998 | 1.00 | 18.34 | | N |
| ANISOU | 1052 | N | ARG | F | 121 | 2116 | 2292 | 2559 | −143 | 32 | −97 | N |
| ATOM | 1053 | CA | ARG | F | 121 | −18.519 | 47.009 | 30.228 | 1.00 | 17.91 | | C |
| ANISOU | 1053 | CA | ARG | F | 121 | 2367 | 2081 | 2354 | −64 | −153 | −106 | C |
| ATOM | 1055 | CB | ARG | F | 121 | −18.202 | 45.699 | 29.480 | 1.00 | 19.34 | | C |
| ANISOU | 1055 | CB | ARG | F | 121 | 2489 | 2421 | 2438 | −145 | −171 | −47 | C |
| ATOM | 1058 | CG | ARG | F | 121 | −17.665 | 44.578 | 30.347 | 1.00 | 19.62 | | C |
| ANISOU | 1058 | CG | ARG | F | 121 | 2477 | 2414 | 2561 | −1 | −91 | −101 | C |
| ATOM | 1061 | CD | ARG | F | 121 | −17.146 | 43.411 | 29.508 | 1.00 | 18.19 | | C |
| ANISOU | 1061 | CD | ARG | F | 121 | 2371 | 2093 | 2445 | −53 | −277 | 9 | C |
| ATOM | 1064 | NE | ARG | F | 121 | −18.241 | 42.661 | 28.903 | 1.00 | 16.81 | | N |
| ANISOU | 1064 | NE | ARG | F | 121 | 2278 | 1934 | 2172 | 42 | −388 | 208 | N |
| ATOM | 1066 | CZ | ARG | F | 121 | −18.125 | 41.443 | 28.380 | 1.00 | 16.78 | | C |
| ANISOU | 1066 | CZ | ARG | F | 121 | 2128 | 2200 | 2046 | 73 | −115 | 55 | C |
| ATOM | 1067 | NH1 | ARG | F | 121 | −16.955 | 40.825 | 28.312 | 1.00 | 17.38 | | N |
| ANISOU | 1067 | NH1 | ARG | F | 121 | 2363 | 2172 | 2065 | 200 | −84 | 4 | N |
| ATOM | 1070 | NH2 | ARG | F | 121 | −19.203 | 40.857 | 27.893 | 1.00 | 16.24 | | N |
| ANISOU | 1070 | NH2 | ARG | F | 121 | 2206 | 2001 | 1964 | −66 | −256 | 132 | N |
| ATOM | 1073 | C | ARG | F | 121 | −19.714 | 46.860 | 31.166 | 1.00 | 19.17 | | C |
| ANISOU | 1073 | C | ARG | F | 121 | 2496 | 2394 | 2393 | −53 | −40 | −94 | C |
| ATOM | 1074 | O | ARG | F | 121 | −19.580 | 46.369 | 32.293 | 1.00 | 21.56 | | O |
| ANISOU | 1074 | O | ARG | F | 121 | 2659 | 2966 | 2566 | −121 | 45 | 30 | O |
| ATOM | 1076 | N | GLU | F | 122 | −20.881 | 47.297 | 30.697 | 1.00 | 20.01 | | N |
| ANISOU | 1076 | N | GLU | F | 122 | 2556 | 2785 | 2261 | 167 | 0 | −246 | N |
| ATOM | 1077 | CA | GLU | F | 122 | −22.066 | 47.418 | 31.558 | 1.00 | 22.57 | | C |
| ANISOU | 1077 | CA | GLU | F | 122 | 2795 | 3001 | 2777 | 47 | 194 | −95 | C |
| ATOM | 1079 | CB | GLU | F | 122 | −23.162 | 48.219 | 30.848 | 1.00 | 28.28 | | C |
| ANISOU | 1079 | CB | GLU | F | 122 | 3478 | 3763 | 3504 | 157 | −3 | 53 | C |
| ATOM | 1082 | CG | GLU | F | 122 | −22.812 | 49.678 | 30.634 | 1.00 | 31.86 | | C |
| ANISOU | 1082 | CG | GLU | F | 122 | 4042 | 4060 | 4003 | 4 | 30 | 71 | C |
| ATOM | 1085 | CD | GLU | F | 122 | −23.787 | 50.504 | 29.946 | 0.00 | 30.00 | | C |
| ANISOU | 1085 | CD | GLU | F | 122 | 3799 | 3799 | 3799 | 0 | 0 | 0 | C |
| ATOM | 1086 | OE1 | GLU | F | 122 | −23.672 | 51.731 | 29.866 | 0.00 | 30.00 | | O |
| ANISOU | 1086 | OE1 | GLU | F | 122 | 3799 | 3799 | 3799 | 0 | 0 | 0 | O |
| ATOM | 1087 | OE2 | GLU | F | 122 | −24.764 | 49.913 | 29.473 | 0.00 | 30.00 | | O |
| ANISOU | 1087 | OE2 | GLU | F | 122 | 3799 | 3799 | 3799 | 0 | 0 | 0 | O |
| ATOM | 1088 | C | GLU | F | 122 | −22.639 | 46.086 | 32.040 | 1.00 | 21.47 | | C |
| ANISOU | 1088 | C | GLU | F | 122 | 2819 | 2759 | 2579 | 28 | 158 | −180 | C |
| ATOM | 1089 | O | GLU | F | 122 | −23.363 | 46.048 | 33.038 | 1.00 | 23.19 | | O |
| ANISOU | 1089 | O | GLU | F | 122 | 2826 | 3107 | 2877 | 70 | 392 | −151 | O |
| ATOM | 1091 | N | ASP | F | 123 | −22.324 | 45.004 | 31.328 | 1.00 | 20.73 | | N |
| ANISOU | 1091 | N | ASP | F | 123 | 2699 | 2711 | 2466 | 69 | 123 | −210 | N |
| ATOM | 1092 | CA | ASP | F | 123 | −22.806 | 43.669 | 31.684 | 1.00 | 21.72 | | C |
| ANISOU | 1092 | CA | ASP | F | 123 | 2711 | 2895 | 2645 | −83 | −30 | −89 | C |
| ATOM | 1094 | CB | ASP | F | 123 | −23.075 | 42.851 | 30.415 | 1.00 | 19.64 | | C |
| ANISOU | 1094 | CB | ASP | F | 123 | 2516 | 2535 | 2411 | −111 | −38 | −124 | C |
| ATOM | 1097 | CG | ASP | F | 123 | −21.823 | 42.590 | 29.593 | 1.00 | 19.69 | | C |
| ANISOU | 1097 | CG | ASP | F | 123 | 2423 | 2508 | 2550 | −178 | −189 | −128 | C |
| ATOM | 1098 | OD1 | ASP | F | 123 | −20.828 | 43.344 | 29.722 | 1.00 | 19.73 | | O |
| ANISOU | 1098 | OD1 | ASP | F | 123 | 2087 | 2711 | 2698 | −161 | −271 | −264 | O |
| ATOM | 1099 | OD2 | ASP | F | 123 | −21.850 | 41.626 | 28.803 | 1.00 | 20.37 | | O |
| ANISOU | 1099 | OD2 | ASP | F | 123 | 2411 | 2743 | 2586 | −31 | −196 | −280 | O |
| ATOM | 1100 | C | ASP | F | 123 | −21.894 | 42.883 | 32.643 | 1.00 | 21.81 | | C |
| ANISOU | 1100 | C | ASP | F | 123 | 2791 | 2886 | 2608 | −123 | −108 | −87 | C |
| ATOM | 1101 | O | ASP | F | 123 | −22.178 | 41.733 | 32.961 | 1.00 | 23.76 | | O |
| ANISOU | 1101 | O | ASP | F | 123 | 2868 | 3169 | 2991 | −93 | −14 | 40 | O |
| ATOM | 1103 | N | HIS | F | 124 | −20.807 | 43.501 | 33.101 | 1.00 | 21.27 | | N |
| ANISOU | 1103 | N | HIS | F | 124 | 2558 | 2862 | 2660 | −224 | 74 | 54 | N |
| ATOM | 1104 | CA | HIS | F | 124 | −19.962 | 42.915 | 34.148 | 1.00 | 22.87 | | C |
| ANISOU | 1104 | CA | HIS | F | 124 | 3027 | 2956 | 2705 | −100 | −122 | −28 | C |
| ATOM | 1106 | CB | HIS | F | 124 | −18.590 | 42.508 | 33.595 | 1.00 | 23.88 | | C |
| ANISOU | 1106 | CB | HIS | F | 124 | 3077 | 3087 | 2909 | −141 | −93 | 23 | C |
| ATOM | 1109 | CG | HIS | F | 124 | −18.605 | 41.243 | 32.790 | 1.00 | 25.03 | | C |
| ANISOU | 1109 | CG | HIS | F | 124 | 3328 | 3149 | 3031 | 65 | −22 | 27 | C |
| ATOM | 1110 | ND1 | HIS | F | 124 | −17.926 | 40.106 | 33.174 | 1.00 | 27.36 | | N |
| ANISOU | 1110 | ND1 | HIS | F | 124 | 3480 | 3510 | 3404 | 165 | −131 | −266 | N |
| ATOM | 1112 | CE1 | HIS | F | 124 | −18.114 | 39.155 | 32.276 | 1.00 | 26.40 | | C |
| ANISOU | 1112 | CE1 | HIS | F | 124 | 3096 | 3487 | 3447 | 128 | −72 | −214 | C |
| ATOM | 1114 | NE2 | HIS | F | 124 | −18.889 | 39.635 | 31.320 | 1.00 | 25.12 | | N |
| ANISOU | 1114 | NE2 | HIS | F | 124 | 3345 | 3196 | 3001 | 52 | 18 | −55 | N |
| ATOM | 1116 | CD2 | HIS | F | 124 | −19.210 | 40.938 | 31.619 | 1.00 | 26.08 | | C |
| ANISOU | 1116 | CD2 | HIS | F | 124 | 3473 | 3272 | 3162 | 160 | −104 | −68 | C |
| ATOM | 1118 | C | HIS | F | 124 | −19.787 | 43.924 | 35.286 | 1.00 | 24.15 | | C |
| ANISOU | 1118 | C | HIS | F | 124 | 3325 | 3049 | 2801 | −82 | −142 | −26 | C |
| ATOM | 1119 | O | HIS | F | 124 | −19.883 | 45.134 | 35.077 | 1.00 | 24.44 | | O |
| ANISOU | 1119 | O | HIS | F | 124 | 3541 | 3043 | 2701 | 18 | −195 | −219 | O |
| ATOM | 1121 | N | ASN | F | 125 | −19.532 | 43.423 | 36.490 | 1.00 | 22.84 | | N |
| ANISOU | 1121 | N | ASN | F | 125 | 3015 | 3057 | 2606 | −458 | −90 | 78 | N |
| ATOM | 1122 | CA | ASN | F | 125 | −19.267 | 44.307 | 37.630 | 1.00 | 23.22 | | C |
| ANISOU | 1122 | CA | ASN | F | 125 | 3101 | 2938 | 2783 | −129 | −63 | −70 | C |
| ATOM | 1124 | CB | ASN | F | 125 | −19.168 | 43.511 | 38.932 | 1.00 | 25.12 | | C |
| ANISOU | 1124 | CB | ASN | F | 125 | 3275 | 3155 | 3111 | −151 | −127 | 12 | C |
| ATOM | 1127 | CG | ASN | F | 125 | −20.496 | 42.955 | 39.380 | 1.00 | 27.67 | | C |
| ANISOU | 1127 | CG | ASN | F | 125 | 3473 | 3577 | 3462 | −21 | 77 | 88 | C |
| ATOM | 1128 | OD1 | ASN | F | 125 | −21.547 | 43.556 | 39.154 | 1.00 | 29.94 | | O |
| ANISOU | 1128 | OD1 | ASN | F | 125 | 3587 | 3936 | 3851 | −15 | 68 | 263 | O |
| ATOM | 1129 | ND2 | ASN | F | 125 | −20.458 | 41.801 | 40.031 | 1.00 | 29.44 | | N |
| ANISOU | 1129 | ND2 | ASN | F | 125 | 3812 | 3642 | 3730 | 30 | −23 | 134 | N |
| ATOM | 1132 | C | ASN | F | 125 | −17.970 | 45.082 | 37.425 | 1.00 | 21.41 | | C |
| ANISOU | 1132 | C | ASN | F | 125 | 3061 | 2603 | 2469 | −88 | 11 | 59 | C |
| ATOM | 1133 | O | ASN | F | 125 | −17.053 | 44.584 | 36.768 | 1.00 | 22.41 | | O |
| ANISOU | 1133 | O | ASN | F | 125 | 3000 | 2525 | 2991 | −157 | −114 | −172 | O |
| ATOM | 1135 | N | PRO | F | 126 | −17.884 | 46.304 | 37.976 | 1.00 | 21.91 | | N |
| ANISOU | 1135 | N | PRO | F | 126 | 2880 | 2956 | 2486 | −150 | −204 | 99 | N |
| ATOM | 1136 | CA | PRO | F | 126 | −16.601 | 46.987 | 37.932 | 1.00 | 21.55 | | C |
| ANISOU | 1136 | CA | PRO | F | 126 | 2696 | 2855 | 2637 | −22 | −41 | 42 | C |
| ATOM | 1138 | CB | PRO | F | 126 | −16.904 | 48.364 | 38.530 | 1.00 | 21.93 | | C |
| ANISOU | 1138 | CB | PRO | F | 126 | 2831 | 2772 | 2727 | −75 | −137 | −48 | C |
| ATOM | 1141 | CG | PRO | F | 126 | −18.115 | 48.171 | 39.349 | 1.00 | 22.95 | | C |
| ANISOU | 1141 | CG | PRO | F | 126 | 3148 | 2703 | 2866 | −36 | −16 | −112 | C |
| ATOM | 1144 | CD | PRO | F | 126 | −18.912 | 47.105 | 38.661 | 1.00 | 22.46 | | C |
| ANISOU | 1144 | CD | PRO | F | 126 | 2737 | 2938 | 2857 | −104 | −154 | −31 | C |
| ATOM | 1147 | C | PRO | F | 126 | −15.575 | 46.243 | 38.770 | 1.00 | 20.61 | | C |
| ANISOU | 1147 | C | PRO | F | 126 | 2640 | 2781 | 2407 | −120 | 0 | −5 | C |
| ATOM | 1148 | O | PRO | F | 126 | −15.939 | 45.511 | 39.697 | 1.00 | 22.12 | | O |
| ANISOU | 1148 | O | PRO | F | 126 | 2580 | 2948 | 2877 | −346 | −335 | 344 | O |
| ATOM | 1149 | N | ILE | F | 127 | −14.307 | 46.403 | 38.416 | 1.00 | 19.44 | | N |
| ANISOU | 1149 | N | ILE | F | 127 | 2446 | 2507 | 2433 | −97 | 14 | −59 | N |
| ATOM | 1150 | CA | ILE | F | 127 | −13.220 | 45.797 | 39.162 | 1.00 | 19.46 | | C |
| ANISOU | 1150 | CA | ILE | F | 127 | 2515 | 2509 | 2367 | −54 | −78 | −29 | C |
| ATOM | 1152 | CB | ILE | F | 127 | −12.063 | 45.354 | 38.224 | 1.00 | 21.19 | | C |
| ANISOU | 1152 | CB | ILE | F | 127 | 2635 | 2805 | 2610 | −37 | 93 | −54 | C |
| ATOM | 1154 | CG1 | ILE | F | 127 | −12.529 | 44.220 | 37.302 | 1.00 | 24.20 | | C |
| ANISOU | 1154 | CG1 | ILE | F | 127 | 2990 | 3222 | 2982 | −5 | 66 | −98 | C |
| ATOM | 1157 | CD1 | ILE | F | 127 | −11.485 | 43.788 | 36.261 | 1.00 | 23.78 | | C |
| ANISOU | 1157 | CD1 | ILE | F | 127 | 2902 | 3129 | 3001 | −4 | 40 | −158 | C |
| ATOM | 1161 | CG2 | ILE | F | 127 | −10.843 | 44.918 | 39.037 | 1.00 | 23.11 | | C |
| ANISOU | 1161 | CG2 | ILE | F | 127 | 2827 | 2905 | 3046 | 82 | 16 | 26 | C |
| ATOM | 1165 | C | ILE | F | 127 | −12.718 | 46.815 | 40.177 | 1.00 | 17.96 | | C |
| ANISOU | 1165 | C | ILE | F | 127 | 2360 | 2095 | 2366 | −145 | 35 | 26 | C |
| ATOM | 1166 | O | ILE | F | 127 | −12.353 | 47.932 | 39.825 | 1.00 | 19.11 | | O |
| ANISOU | 1166 | O | ILE | F | 127 | 2439 | 2158 | 2661 | −414 | −114 | 76 | O |
| ATOM | 1168 | N | SER | F | 128 | −12.710 | 46.422 | 41.443 | 1.00 | 17.99 | | N |
| ANISOU | 1168 | N | SER | F | 128 | 2182 | 2285 | 2366 | −70 | −99 | 166 | N |
| ATOM | 1169 | CA | SER | F | 128 | −12.121 | 47.242 | 42.496 | 1.00 | 17.94 | | C |
| ANISOU | 1169 | CA | SER | F | 128 | 2311 | 2011 | 2494 | −2 | 45 | −98 | C |
| ATOM | 1171 | CB | ASER | F | 128 | −12.573 | 46.740 | 43.871 | 0.50 | 18.69 | | C |
| ANISOU | 1171 | CB | ASER | F | 128 | 2311 | 2400 | 2386 | 53 | 7 | −53 | C |
| ATOM | 1172 | CB | BSER | F | 128 | −12.578 | 46.769 | 43.874 | 0.50 | 18.83 | | C |
| ANISOU | 1172 | CB | BSER | F | 128 | 2336 | 2384 | 2432 | 19 | −2 | −51 | C |
| ATOM | 1177 | OG | ASER | F | 128 | −12.022 | 47.508 | 44.928 | 0.50 | 20.18 | | O |
| ANISOU | 1177 | OG | ASER | F | 128 | 2515 | 2689 | 2464 | 86 | −11 | −31 | O |
| ATOM | 1178 | OG | BSER | F | 128 | −13.976 | 46.946 | 44.025 | 0.50 | 20.88 | | O |
| ANISOU | 1178 | OG | BSER | F | 128 | 2440 | 2648 | 2845 | 17 | 31 | 35 | O |
| ATOM | 1181 | C | SER | F | 128 | −10.602 | 47.167 | 42.379 | 1.00 | 18.74 | | C |
| ANISOU | 1181 | C | SER | F | 128 | 2255 | 2284 | 2582 | 25 | 3 | 16 | C |
| ATOM | 1182 | O | SER | F | 128 | −10.024 | 46.095 | 42.417 | 1.00 | 19.81 | | O |
| ANISOU | 1182 | O | SER | F | 128 | 1992 | 2165 | 3369 | 119 | −168 | −172 | O |
| ATOM | 1184 | N | ILE | F | 129 | −9.960 | 48.313 | 42.215 | 1.00 | 17.19 | | N |
| ANISOU | 1184 | N | ILE | F | 129 | 1914 | 2080 | 2535 | 60 | −12 | −89 | N |
| ATOM | 1185 | CA | ILE | F | 129 | −8.524 | 48.365 | 41.959 | 1.00 | 17.26 | | C |
| ANISOU | 1185 | CA | ILE | F | 129 | 2171 | 2227 | 2157 | 32 | 100 | −10 | C |
| ATOM | 1187 | CB | AILE | F | 129 | −8.150 | 49.628 | 41.150 | 0.40 | 18.33 | | C |
| ANISOU | 1187 | CB | AILE | F | 129 | 2259 | 2280 | 2422 | −29 | 61 | −28 | C |
| ATOM | 1188 | CB | BILE | F | 129 | −8.144 | 49.613 | 41.154 | 0.60 | 18.29 | | C |
| ANISOU | 1188 | CB | BILE | F | 129 | 2249 | 2264 | 2435 | −55 | 54 | −45 | C |
| ATOM | 1191 | CG1 | AILE | F | 129 | −9.034 | 49.747 | 39.903 | 0.40 | 18.91 | | C |
| ANISOU | 1191 | CG1 | AILE | F | 129 | 2310 | 2451 | 2421 | −32 | 43 | −15 | C |
| ATOM | 1192 | CG1 | BILE | F | 129 | −8.806 | 49.545 | 39.777 | 0.60 | 18.63 | | C |
| ANISOU | 1192 | CG1 | BILE | F | 129 | 2317 | 2326 | 2434 | −66 | −45 | 2 | C |
| ATOM | 1197 | CD1 | AILE | F | 129 | −8.915 | 48.587 | 38.936 | 0.40 | 19.61 | | C |
| ANISOU | 1197 | CD1 | AILE | F | 129 | 2370 | 2597 | 2484 | −18 | −15 | 1 | C |
| ATOM | 1198 | CD1 | BILE | F | 129 | −8.632 | 50.803 | 38.955 | 0.60 | 17.90 | | C |
| ANISOU | 1198 | CD1 | BILE | F | 129 | 2211 | 2415 | 2174 | −140 | −71 | 134 | C |
| ATOM | 1205 | CG2 | AILE | F | 129 | −6.670 | 49.607 | 40.763 | 0.40 | 18.88 | | C |
| ANISOU | 1205 | CG2 | AILE | F | 129 | 2249 | 2306 | 2618 | −67 | 21 | −12 | C |
| ATOM | 1206 | CG2 | BILE | F | 129 | −6.619 | 49.728 | 41.029 | 0.60 | 19.18 | | C |
| ANISOU | 1206 | CG2 | BILE | F | 129 | 2258 | 2371 | 2656 | −143 | −7 | −59 | C |
| ATOM | 1213 | C | ILE | F | 129 | −7.757 | 48.353 | 43.277 | 1.00 | 18.10 | | C |
| ANISOU | 1213 | C | ILE | F | 129 | 2183 | 2428 | 2264 | 10 | 57 | 138 | C |
| ATOM | 1214 | O | ILE | F | 129 | −7.954 | 49.233 | 44.100 | 1.00 | 19.43 | | O |
| ANISOU | 1214 | O | ILE | F | 129 | 2047 | 2761 | 2574 | 118 | −108 | −239 | O |
| ATOM | 1216 | N | PRO | F | 130 | −6.864 | 47.365 | 43.465 | 1.00 | 18.06 | | N |
| ANISOU | 1216 | N | PRO | F | 130 | 2218 | 2307 | 2336 | −54 | 67 | 94 | N |
| ATOM | 1217 | CA | PRO | F | 130 | −6.051 | 47.316 | 44.671 | 1.00 | 18.76 | | C |
| ANISOU | 1217 | CA | PRO | F | 130 | 2258 | 2406 | 2464 | 17 | 76 | −6 | C |
| ATOM | 1219 | CB | PRO | F | 130 | −5.493 | 45.893 | 44.645 | 1.00 | 20.84 | | C |
| ANISOU | 1219 | CB | PRO | F | 130 | 2459 | 2485 | 2970 | 59 | 19 | 151 | C |
| ATOM | 1222 | CG | PRO | F | 130 | −5.365 | 45.600 | 43.199 | 1.00 | 21.00 | | C |
| ANISOU | 1222 | CG | PRO | F | 130 | 2496 | 2379 | 3103 | 13 | 6 | 36 | C |
| ATOM | 1225 | CD | PRO | F | 130 | −6.542 | 46.252 | 42.554 | 1.00 | 19.45 | | C |
| ANISOU | 1225 | CD | PRO | F | 130 | 2378 | 2335 | 2676 | −98 | 45 | 17 | C |
| ATOM | 1228 | C | PRO | F | 130 | −4.912 | 48.321 | 44.611 | 1.00 | 17.79 | | C |
| ANISOU | 1228 | C | PRO | F | 130 | 2246 | 2512 | 1999 | −80 | −93 | 84 | C |
| ATOM | 1229 | O | PRO | F | 130 | −4.418 | 48.628 | 43.528 | 1.00 | 19.43 | | O |
| ANISOU | 1229 | O | PRO | F | 130 | 2320 | 2582 | 2481 | −37 | 98 | −58 | O |
| ATOM | 1230 | N | MET | F | 131 | −4.511 | 48.832 | 45.769 | 1.00 | 18.34 | | N |
| ANISOU | 1230 | N | MET | F | 131 | 2210 | 2273 | 2485 | −35 | 4 | −27 | N |
| ATOM | 1231 | CA | MET | F | 131 | −3.293 | 49.614 | 45.864 | 1.00 | 19.95 | | C |
| ANISOU | 1231 | CA | MET | F | 131 | 2297 | 2492 | 2789 | 90 | −61 | 123 | C |
| ATOM | 1233 | CB | MET | F | 131 | −3.405 | 50.640 | 46.980 | 1.00 | 19.87 | | C |
| ANISOU | 1233 | CB | MET | F | 131 | 2308 | 2650 | 2588 | −4 | −44 | 186 | C |
| ATOM | 1236 | CG | MET | F | 131 | −4.586 | 51.565 | 46.820 | 1.00 | 22.06 | | C |
| ANISOU | 1236 | CG | MET | F | 131 | 2583 | 2697 | 3101 | 89 | 36 | −90 | C |
| ATOM | 1239 | SD | MET | F | 131 | −4.418 | 53.013 | 47.851 | 1.00 | 21.20 | | S |
| ANISOU | 1239 | SD | MET | F | 131 | 2225 | 2683 | 3145 | 316 | 64 | −214 | S |
| ATOM | 1240 | CE | MET | F | 131 | −3.384 | 54.057 | 46.844 | 1.00 | 19.42 | | C |
| ANISOU | 1240 | CE | MET | F | 131 | 2415 | 2386 | 2577 | 108 | −56 | −48 | C |
| ATOM | 1244 | C | MET | F | 131 | −2.133 | 48.680 | 46.143 | 1.00 | 20.50 | | C |
| ANISOU | 1244 | C | MET | F | 131 | 2291 | 2517 | 2979 | 125 | 49 | 279 | C |
| ATOM | 1245 | O | MET | F | 131 | −2.332 | 47.550 | 46.586 | 1.00 | 20.30 | | O |
| ANISOU | 1245 | O | MET | F | 131 | 1867 | 2450 | 3395 | 178 | 251 | 343 | O |
| ATOM | 1247 | N | LEU | F | 132 | −0.924 | 49.153 | 45.865 | 1.00 | 19.46 | | N |
| ANISOU | 1247 | N | LEU | F | 132 | 2255 | 2353 | 2784 | 144 | −75 | 382 | N |
| ATOM | 1248 | CA | LEU | F | 132 | 0.277 | 48.450 | 46.293 | 1.00 | 20.50 | | C |
| ANISOU | 1248 | CA | LEU | F | 132 | 2360 | 2631 | 2796 | 121 | 76 | 240 | C |
| ATOM | 1250 | CB | LEU | F | 132 | 1.523 | 49.067 | 45.646 | 1.00 | 21.67 | | C |
| ANISOU | 1250 | CB | LEU | F | 132 | 2537 | 2820 | 2873 | 56 | 145 | 118 | C |
| ATOM | 1253 | CG | LEU | F | 132 | 1.559 | 49.057 | 44.106 | 1.00 | 21.85 | | C |
| ANISOU | 1253 | CG | LEU | F | 132 | 2624 | 2805 | 2871 | 9 | 124 | −71 | C |
| ATOM | 1255 | CD1 | LEU | F | 132 | 2.896 | 49.578 | 43.596 | 1.00 | 22.50 | | C |
| ANISOU | 1255 | CD1 | LEU | F | 132 | 2703 | 2764 | 3080 | 91 | 125 | −103 | C |
| ATOM | 1259 | CD2 | LEU | F | 132 | 1.272 | 47.678 | 43.516 | 1.00 | 23.34 | | C |
| ANISOU | 1259 | CD2 | LEU | F | 132 | 2869 | 2950 | 3049 | 73 | 267 | −45 | C |
| ATOM | 1263 | C | LEU | F | 132 | 0.348 | 48.510 | 47.821 | 1.00 | 21.98 | | C |
| ANISOU | 1263 | C | LEU | F | 132 | 2672 | 2777 | 2899 | 104 | 52 | 86 | C |
| ATOM | 1264 | O | LEU | F | 132 | −0.370 | 49.288 | 48.461 | 1.00 | 21.59 | | O |
| ANISOU | 1264 | O | LEU | F | 132 | 2392 | 3083 | 2727 | 202 | 126 | 162 | O |
| ATOM | 1266 | N | ASN | F | 133 | 1.213 | 47.692 | 48.408 | 1.00 | 23.94 | | N |
| ANISOU | 1266 | N | ASN | F | 133 | 2854 | 3087 | 3154 | 53 | −8 | 251 | N |
| ATOM | 1267 | CA | ASN | F | 133 | 1.246 | 47.543 | 49.867 | 1.00 | 24.54 | | C |
| ANISOU | 1267 | CA | ASN | F | 133 | 3069 | 3208 | 3046 | 94 | 17 | 184 | C |
| ATOM | 1269 | CB | ASN | F | 133 | 2.174 | 46.393 | 50.272 | 1.00 | 27.81 | | C |
| ANISOU | 1269 | CB | ASN | F | 133 | 3420 | 3430 | 3714 | 144 | 41 | 164 | C |
| ATOM | 1272 | CG | ASN | F | 133 | 1.595 | 45.030 | 49.930 | 1.00 | 30.45 | | C |
| ANISOU | 1272 | CG | ASN | F | 133 | 3811 | 3794 | 3963 | 9 | −36 | 61 | C |
| ATOM | 1273 | OD1 | ASN | F | 133 | 0.442 | 44.919 | 49.506 | 1.00 | 33.10 | | O |
| ANISOU | 1273 | OD1 | ASN | F | 133 | 4003 | 4133 | 4440 | 58 | −36 | 190 | O |
| ATOM | 1274 | ND2 | ASN | F | 133 | 2.391 | 43.983 | 50.118 | 1.00 | 32.68 | | N |
| ANISOU | 1274 | ND2 | ASN | F | 133 | 3964 | 4080 | 4372 | 116 | −13 | 76 | N |
| ATOM | 1277 | C | ASN | F | 133 | 1.601 | 48.808 | 50.650 | 1.00 | 25.14 | | C |
| ANISOU | 1277 | C | ASN | F | 133 | 3146 | 3378 | 3027 | 46 | −83 | 110 | C |
| ATOM | 1278 | O | ASN | F | 133 | 1.295 | 48.898 | 51.840 | 1.00 | 27.95 | | O |
| ANISOU | 1278 | O | ASN | F | 133 | 3543 | 3777 | 3298 | 106 | 62 | 207 | O |
| ATOM | 1280 | N | ASP | F | 134 | 2.219 | 49.789 | 49.990 | 1.00 | 24.82 | | N |
| ANISOU | 1280 | N | ASP | F | 134 | 3105 | 3339 | 2986 | 46 | −174 | 160 | N |
| ATOM | 1281 | CA | ASP | F | 134 | 2.525 | 51.066 | 50.638 | 1.00 | 25.10 | | C |
| ANISOU | 1281 | CA | ASP | F | 134 | 3108 | 3239 | 3189 | 77 | −3 | 120 | C |
| ATOM | 1283 | CB | ASP | F | 134 | 3.639 | 51.832 | 49.890 | 1.00 | 27.25 | | C |
| ANISOU | 1283 | CB | ASP | F | 134 | 3337 | 3501 | 3515 | 54 | 54 | 219 | C |
| ATOM | 1286 | CG | ASP | F | 134 | 3.238 | 52.297 | 48.487 | 1.00 | 28.42 | | C |
| ANISOU | 1286 | CG | ASP | F | 134 | 3391 | 3737 | 3668 | −36 | −39 | 144 | C |
| ATOM | 1287 | OD1 | ASP | F | 134 | 2.086 | 52.091 | 48.044 | 1.00 | 25.50 | | O |
| ANISOU | 1287 | OD1 | ASP | F | 134 | 3193 | 3236 | 3260 | 83 | −171 | 182 | O |
| ATOM | 1288 | OD2 | ASP | F | 134 | 4.113 | 52.892 | 47.822 | 1.00 | 30.05 | | O |
| ANISOU | 1288 | OD2 | ASP | F | 134 | 3528 | 3997 | 3893 | −29 | −54 | 443 | O |
| ATOM | 1289 | C | ASP | F | 134 | 1.297 | 51.961 | 50.878 | 1.00 | 24.77 | | C |
| ANISOU | 1289 | C | ASP | F | 134 | 3113 | 3208 | 3088 | 34 | −43 | 58 | C |
| ATOM | 1290 | O | ASP | F | 134 | 1.382 | 52.933 | 51.628 | 1.00 | 26.84 | | O |
| ANISOU | 1290 | O | ASP | F | 134 | 3540 | 3310 | 3345 | −126 | −16 | 54 | O |
| ATOM | 1292 | N | GLY | F | 135 | 0.166 | 51.630 | 50.254 | 1.00 | 23.76 | | N |
| ANISOU | 1292 | N | GLY | F | 135 | 3056 | 3110 | 2859 | 127 | −54 | −71 | N |
| ATOM | 1293 | CA | GLY | F | 135 | −1.070 | 52.400 | 50.420 | 1.00 | 22.21 | | C |
| ANISOU | 1293 | CA | GLY | F | 135 | 2933 | 2970 | 2535 | 49 | 13 | −73 | C |
| ATOM | 1296 | C | GLY | F | 135 | −1.039 | 53.756 | 49.735 | 1.00 | 22.44 | | C |
| ANISOU | 1296 | C | GLY | F | 135 | 2803 | 2862 | 2861 | −2 | 98 | −176 | C |
| ATOM | 1297 | O | GLY | F | 135 | −1.869 | 54.620 | 50.027 | 1.00 | 22.98 | | O |
| ANISOU | 1297 | O | GLY | F | 135 | 2823 | 2760 | 3148 | 12 | 227 | −198 | O |
| ATOM | 1299 | N | ARG | F | 136 | −0.094 | 53.934 | 48.813 | 1.00 | 20.80 | | N |
| ANISOU | 1299 | N | ARG | F | 136 | 2657 | 2716 | 2528 | −83 | 97 | −109 | N |
| ATOM | 1300 | CA | ARG | F | 136 | 0.134 | 55.219 | 48.150 | 1.00 | 21.72 | | C |
| ANISOU | 1300 | CA | ARG | F | 136 | 2848 | 2766 | 2639 | 137 | 126 | −53 | C |
| ATOM | 1302 | CB | ARG | F | 136 | 1.454 | 55.825 | 48.633 | 1.00 | 23.16 | | C |
| ANISOU | 1302 | CB | ARG | F | 136 | 2971 | 2827 | 3001 | 11 | 31 | −41 | C |
| ATOM | 1305 | CG | ARG | F | 136 | 1.591 | 55.896 | 50.151 | 1.00 | 24.12 | | C |
| ANISOU | 1305 | CG | ARG | F | 136 | 3078 | 2994 | 3092 | 7 | −16 | −145 | C |
| ATOM | 1308 | CD | ARG | F | 136 | 2.729 | 56.800 | 50.606 | 1.00 | 25.79 | | C |
| ANISOU | 1308 | CD | ARG | F | 136 | 3154 | 3192 | 3453 | 3 | −61 | −21 | C |
| ATOM | 1311 | NE | ARG | F | 136 | 2.747 | 56.926 | 52.062 | 1.00 | 27.74 | | N |
| ANISOU | 1311 | NE | ARG | F | 136 | 3539 | 3464 | 3537 | −5 | 1 | −53 | N |
| ATOM | 1313 | CZ | ARG | F | 136 | 3.567 | 57.718 | 52.754 | 1.00 | 30.25 | | C |
| ANISOU | 1313 | CZ | ARG | F | 136 | 3804 | 3796 | 3892 | −29 | −14 | −24 | C |
| ATOM | 1314 | NH1 | ARG | F | 136 | 4.464 | 58.478 | 52.132 | 1.00 | 31.93 | | N |
| ANISOU | 1314 | NH1 | ARG | F | 136 | 4022 | 3995 | 4112 | −43 | 82 | 92 | N |
| ATOM | 1317 | NH2 | ARG | F | 136 | 3.491 | 57.752 | 54.082 | 1.00 | 30.74 | | N |
| ANISOU | 1317 | NH2 | ARG | F | 136 | 3831 | 3888 | 3957 | 9 | 1 | 66 | N |
| ATOM | 1320 | C | ARG | F | 136 | 0.172 | 55.125 | 46.626 | 1.00 | 19.97 | | C |
| ANISOU | 1320 | C | ARG | F | 136 | 2738 | 2422 | 2427 | 379 | 163 | −103 | C |
| ATOM | 1321 | O | ARG | F | 136 | −0.018 | 56.123 | 45.946 | 1.00 | 25.10 | | O |
| ANISOU | 1321 | O | ARG | F | 136 | 3861 | 2732 | 2943 | 877 | 359 | −127 | O |
| ATOM | 1323 | N | ARG | F | 137 | 0.448 | 53.942 | 46.088 | 1.00 | 17.28 | | N |
| ANISOU | 1323 | N | ARG | F | 137 | 2172 | 2035 | 2355 | 144 | −97 | −162 | N |
| ATOM | 1324 | CA | ARG | F | 137 | 0.568 | 53.768 | 44.654 | 1.00 | 17.46 | | C |
| ANISOU | 1324 | CA | ARG | F | 137 | 2136 | 2179 | 2315 | 183 | −53 | 5 | C |
| ATOM | 1326 | CB | ARG | F | 137 | 1.992 | 53.351 | 44.279 | 1.00 | 18.11 | | C |
| ANISOU | 1326 | CB | ARG | F | 137 | 2237 | 2243 | 2401 | 94 | 69 | −86 | C |
| ATOM | 1329 | CG | ARG | F | 137 | 3.034 | 54.405 | 44.588 | 1.00 | 19.93 | | C |
| ANISOU | 1329 | CG | ARG | F | 137 | 2338 | 2487 | 2747 | −36 | 71 | −163 | C |
| ATOM | 1332 | CD | ARG | F | 137 | 4.396 | 54.015 | 44.052 | 1.00 | 23.05 | | C |
| ANISOU | 1332 | CD | ARG | F | 137 | 2642 | 2960 | 3154 | 22 | 109 | −15 | C |
| ATOM | 1335 | NE | ARG | F | 137 | 4.895 | 52.793 | 44.680 | 1.00 | 25.64 | | N |
| ANISOU | 1335 | NE | ARG | F | 137 | 2918 | 3279 | 3545 | 68 | 130 | −50 | N |
| ATOM | 1337 | CZ | ARG | F | 137 | 5.604 | 51.849 | 44.058 | 1.00 | 25.30 | | C |
| ANISOU | 1337 | CZ | ARG | F | 137 | 3209 | 2990 | 3413 | 19 | 53 | −61 | C |
| ATOM | 1338 | NH1 | ARG | F | 137 | 5.895 | 51.950 | 42.763 | 1.00 | 25.69 | | N |
| ANISOU | 1338 | NH1 | ARG | F | 137 | 3124 | 2992 | 3645 | 270 | 388 | −47 | N |
| ATOM | 1341 | NH2 | ARG | F | 137 | 6.002 | 50.787 | 44.743 | 1.00 | 27.37 | | N |
| ANISOU | 1341 | NH2 | ARG | F | 137 | 3164 | 3347 | 3888 | 120 | 210 | −5 | N |
| ATOM | 1344 | C | ARG | F | 137 | −0.395 | 52.714 | 44.139 | 1.00 | 15.06 | | C |
| ANISOU | 1344 | C | ARG | F | 137 | 1802 | 1925 | 1994 | 80 | 124 | −6 | C |
| ATOM | 1345 | O | ARG | F | 137 | −0.763 | 51.783 | 44.850 | 1.00 | 15.24 | | O |
| ANISOU | 1345 | O | ARG | F | 137 | 1768 | 2087 | 1933 | 221 | 152 | 69 | O |
| ATOM | 1347 | N | ILE | F | 138 | −0.782 | 52.885 | 42.882 | 1.00 | 14.86 | | N |
| ANISOU | 1347 | N | ILE | F | 138 | 1987 | 1888 | 1769 | 27 | −13 | −54 | N |
| ATOM | 1348 | CA | ILE | F | 138 | −1.470 | 51.863 | 42.102 | 1.00 | 15.16 | | C |
| ANISOU | 1348 | CA | ILE | F | 138 | 1815 | 1885 | 2058 | 22 | −2 | 82 | C |
| ATOM | 1350 | CB | ILE | F | 138 | −2.869 | 52.348 | 41.656 | 1.00 | 15.14 | | C |
| ANISOU | 1350 | CB | ILE | F | 138 | 1862 | 1910 | 1977 | 66 | 49 | 103 | C |
| ATOM | 1352 | CG1 | ILE | F | 138 | −3.757 | 52.532 | 42.896 | 1.00 | 16.80 | | C |
| ANISOU | 1352 | CG1 | ILE | F | 138 | 2284 | 2313 | 1784 | 104 | 79 | 261 | C |
| ATOM | 1355 | CD1 | ILE | F | 138 | −5.143 | 53.056 | 42.628 | 1.00 | 17.39 | | C |
| ANISOU | 1355 | CD1 | ILE | F | 138 | 2039 | 2233 | 2332 | 9 | 89 | 173 | C |
| ATOM | 1359 | CG2 | ILE | F | 138 | −3.484 | 51.390 | 40.629 | 1.00 | 16.48 | | C |
| ANISOU | 1359 | CG2 | ILE | F | 138 | 1829 | 2141 | 2288 | 1 | 62 | −12 | C |
| ATOM | 1363 | C | ILE | F | 138 | −0.618 | 51.595 | 40.874 | 1.00 | 15.24 | | C |
| ANISOU | 1363 | C | ILE | F | 138 | 1761 | 1888 | 2139 | 69 | 133 | −104 | C |
| ATOM | 1364 | O | ILE | F | 138 | −0.147 | 52.531 | 40.234 | 1.00 | 15.00 | | O |
| ANISOU | 1364 | O | ILE | F | 138 | 1931 | 1749 | 2019 | 145 | 59 | −26 | O |
| ATOM | 1366 | N | VAL | F | 139 | −0.391 | 50.315 | 40.583 | 1.00 | 15.93 | | N |
| ANISOU | 1366 | N | VAL | F | 139 | 1992 | 1837 | 2223 | 216 | −54 | 116 | N |
| ATOM | 1367 | CA | VAL | F | 139 | 0.166 | 49.865 | 39.302 | 1.00 | 16.50 | | C |
| ANISOU | 1367 | CA | VAL | F | 139 | 2025 | 1952 | 2289 | −1 | −104 | 16 | C |
| ATOM | 1369 | CB | VAL | F | 139 | 1.700 | 49.603 | 39.357 | 1.00 | 17.84 | | C |
| ANISOU | 1369 | CB | VAL | F | 139 | 2105 | 2322 | 2351 | 217 | −16 | 28 | C |
| ATOM | 1371 | CG1 | VAL | F | 139 | 2.220 | 49.226 | 37.968 | 1.00 | 19.59 | | C |
| ANISOU | 1371 | CG1 | VAL | F | 139 | 2227 | 2691 | 2526 | 319 | 187 | −62 | C |
| ATOM | 1375 | CG2 | VAL | F | 139 | 2.462 | 50.800 | 39.898 | 1.00 | 18.51 | | C |
| ANISOU | 1375 | CG2 | VAL | F | 139 | 2230 | 2380 | 2423 | 40 | −13 | 113 | C |
| ATOM | 1379 | C | VAL | F | 139 | −0.549 | 48.550 | 39.000 | 1.00 | 16.60 | | C |
| ANISOU | 1379 | C | VAL | F | 139 | 2030 | 2037 | 2239 | −104 | −169 | −33 | C |
| ATOM | 1380 | O | VAL | F | 139 | −0.175 | 47.499 | 39.529 | 1.00 | 21.65 | | O |
| ANISOU | 1380 | O | VAL | F | 139 | 2841 | 2294 | 3088 | 55 | −413 | 96 | O |
| ATOM | 1382 | N | PHE | F | 140 | −1.583 | 48.619 | 38.167 | 1.00 | 15.70 | | N |
| ANISOU | 1382 | N | PHE | F | 140 | 2139 | 1757 | 2068 | 135 | −92 | −51 | N |
| ATOM | 1383 | CA | PHE | F | 140 | −2.560 | 47.534 | 38.053 | 1.00 | 16.70 | | C |
| ANISOU | 1383 | CA | PHE | F | 140 | 2182 | 1995 | 2169 | 3 | −89 | 6 | C |
| ATOM | 1385 | CB | PHE | F | 140 | −3.846 | 47.938 | 38.770 | 1.00 | 20.87 | | C |
| ANISOU | 1385 | CB | PHE | F | 140 | 2707 | 2552 | 2668 | −62 | 128 | −187 | C |
| ATOM | 1388 | CG | PHE | F | 140 | −4.820 | 46.812 | 38.954 | 1.00 | 20.87 | | C |
| ANISOU | 1388 | CG | PHE | F | 140 | 2600 | 2932 | 2397 | −39 | 369 | −398 | C |
| ATOM | 1389 | CD1 | PHE | F | 140 | −4.481 | 45.702 | 39.703 | 1.00 | 23.86 | | C |
| ANISOU | 1389 | CD1 | PHE | F | 140 | 2897 | 2676 | 3490 | −282 | 206 | −255 | C |
| ATOM | 1391 | CE1 | PHE | F | 140 | −5.393 | 44.652 | 39.880 | 1.00 | 24.36 | | C |
| ANISOU | 1391 | CE1 | PHE | F | 140 | 3010 | 2695 | 3548 | −172 | 125 | −41 | C |
| ATOM | 1393 | CZ | PHE | F | 140 | −6.655 | 44.746 | 39.321 | 1.00 | 22.92 | | C |
| ANISOU | 1393 | CZ | PHE | F | 140 | 2876 | 2734 | 3095 | −227 | 145 | −299 | C |
| ATOM | 1395 | CE2 | PHE | F | 140 | −7.004 | 45.853 | 38.584 | 1.00 | 23.95 | | C |
| ANISOU | 1395 | CE2 | PHE | F | 140 | 2937 | 3032 | 3129 | −223 | 207 | −348 | C |
| ATOM | 1397 | CD2 | PHE | F | 140 | −6.086 | 46.885 | 38.405 | 1.00 | 22.59 | | C |
| ANISOU | 1397 | CD2 | PHE | F | 140 | 2953 | 2795 | 2835 | −111 | 200 | −374 | C |
| ATOM | 1399 | C | PHE | F | 140 | −2.865 | 47.241 | 36.596 | 1.00 | 15.17 | | C |
| ANISOU | 1399 | C | PHE | F | 140 | 1902 | 1969 | 1891 | 71 | −74 | 0 | C |
| ATOM | 1400 | O | PHE | F | 140 | −3.092 | 48.158 | 35.814 | 1.00 | 16.11 | | O |
| ANISOU | 1400 | O | PHE | F | 140 | 2310 | 1792 | 2017 | 15 | −57 | −13 | O |
| ATOM | 1402 | N | THR | F | 141 | −2.879 | 45.958 | 36.245 | 1.00 | 14.78 | | N |
| ANISOU | 1402 | N | THR | F | 141 | 1945 | 1722 | 1948 | −16 | 81 | −95 | N |
| ATOM | 1403 | CA | THR | F | 141 | −3.123 | 45.500 | 34.883 | 1.00 | 15.54 | | C |
| ANISOU | 1403 | CA | THR | F | 141 | 1987 | 1936 | 1979 | −49 | 1 | 112 | C |
| ATOM | 1405 | CB | ATHR | F | 141 | −1.877 | 44.777 | 34.342 | 0.65 | 18.27 | | C |
| ANISOU | 1405 | CB | ATHR | F | 141 | 2209 | 2356 | 2376 | −37 | 50 | −138 | C |
| ATOM | 1406 | CB | BTHR | F | 141 | −1.904 | 44.765 | 34.264 | 0.35 | 18.01 | | C |
| ANISOU | 1406 | CB | BTHR | F | 141 | 2183 | 2342 | 2315 | −27 | 18 | −67 | C |
| ATOM | 1409 | OG1 | ATHR | F | 141 | −0.728 | 45.615 | 34.521 | 0.65 | 17.99 | | O |
| ANISOU | 1409 | OG1 | ATHR | F | 141 | 2050 | 2371 | 2412 | 40 | 358 | −179 | O |
| ATOM | 1410 | OG1 | BTHR | F | 141 | −1.481 | 43.698 | 35.120 | 0.35 | 18.84 | | O |
| ANISOU | 1410 | OG1 | BTHR | F | 141 | 2254 | 2661 | 2241 | −22 | −68 | −41 | O |
| ATOM | 1413 | CG2 | ATHR | F | 141 | −2.044 | 44.431 | 32.871 | 0.65 | 18.25 | | C |
| ANISOU | 1413 | CG2 | ATHR | F | 141 | 2280 | 2334 | 2321 | −79 | 113 | −267 | C |
| ATOM | 1414 | CG2 | BTHR | F | 141 | −0.756 | 45.713 | 34.039 | 0.35 | 18.09 | | C |
| ANISOU | 1414 | CG2 | BTHR | F | 141 | 2277 | 2238 | 2355 | −7 | 83 | −30 | C |
| ATOM | 1421 | C | THR | F | 141 | −4.309 | 44.546 | 34.840 | 1.00 | 14.50 | | C |
| ANISOU | 1421 | C | THR | F | 141 | 1884 | 1745 | 1881 | 34 | −19 | −149 | C |
| ATOM | 1422 | O | THR | F | 141 | −4.378 | 43.579 | 35.613 | 1.00 | 15.83 | | O |
| ANISOU | 1422 | O | THR | F | 141 | 2124 | 1891 | 1996 | −69 | −24 | 89 | O |
| ATOM | 1424 | N | VAL | F | 142 | −5.212 | 44.823 | 33.909 | 1.00 | 15.64 | | N |
| ANISOU | 1424 | N | VAL | F | 142 | 2108 | 1947 | 1886 | −16 | 26 | 15 | N |
| ATOM | 1425 | CA | VAL | F | 142 | −6.358 | 43.978 | 33.614 | 1.00 | 16.86 | | C |
| ANISOU | 1425 | CA | VAL | F | 142 | 2202 | 2125 | 2079 | −69 | −28 | 51 | C |
| ATOM | 1427 | CB | VAL | F | 142 | −7.671 | 44.752 | 33.940 | 1.00 | 21.78 | | C |
| ANISOU | 1427 | CB | VAL | F | 142 | 2611 | 3217 | 2444 | 1 | 392 | −179 | C |
| ATOM | 1429 | CG1 | VAL | F | 142 | −8.903 | 44.082 | 33.354 | 1.00 | 24.73 | | C |
| ANISOU | 1429 | CG1 | VAL | F | 142 | 2892 | 3569 | 2933 | 227 | 185 | −42 | C |
| ATOM | 1433 | CG2 | VAL | F | 142 | −7.807 | 44.922 | 35.457 | 1.00 | 21.96 | | C |
| ANISOU | 1433 | CG2 | VAL | F | 142 | 2688 | 3282 | 2371 | −131 | 99 | −202 | C |
| ATOM | 1437 | C | VAL | F | 142 | −6.308 | 43.639 | 32.126 | 1.00 | 15.68 | | C |
| ANISOU | 1437 | C | VAL | F | 142 | 1920 | 1989 | 2046 | −65 | −126 | 9 | C |
| ATOM | 1438 | O | VAL | F | 142 | −5.833 | 44.435 | 31.323 | 1.00 | 16.32 | | O |
| ANISOU | 1438 | O | VAL | F | 142 | 2183 | 2059 | 1958 | −278 | 120 | −16 | O |
| ATOM | 1440 | N | VAL | F | 143 | −6.767 | 42.445 | 31.769 | 1.00 | 14.19 | | N |
| ANISOU | 1440 | N | VAL | F | 143 | 1604 | 1827 | 1959 | −27 | −81 | 188 | N |
| ATOM | 1441 | CA | VAL | F | 143 | −7.064 | 42.112 | 30.388 | 1.00 | 14.51 | | C |
| ANISOU | 1441 | CA | VAL | F | 143 | 1682 | 1711 | 2120 | −174 | 44 | −22 | C |
| ATOM | 1443 | CB | VAL | F | 143 | −6.285 | 40.885 | 29.895 | 1.00 | 16.39 | | C |
| ANISOU | 1443 | CB | VAL | F | 143 | 1862 | 1909 | 2454 | −11 | 0 | 108 | C |
| ATOM | 1445 | CG1 | VAL | F | 143 | −6.573 | 40.641 | 28.409 | 1.00 | 17.26 | | C |
| ANISOU | 1445 | CG1 | VAL | F | 143 | 2054 | 1975 | 2528 | 85 | 259 | −91 | C |
| ATOM | 1449 | CG2 | VAL | F | 143 | −4.790 | 41.079 | 30.144 | 1.00 | 18.30 | | C |
| ANISOU | 1449 | CG2 | VAL | F | 143 | 2052 | 2188 | 2711 | 84 | 103 | 398 | C |
| ATOM | 1453 | C | VAL | F | 143 | −8.559 | 41.863 | 30.302 | 1.00 | 14.41 | | C |
| ANISOU | 1453 | C | VAL | F | 143 | 1728 | 1782 | 1965 | 116 | 130 | 56 | C |
| ATOM | 1454 | O | VAL | F | 143 | −9.122 | 41.164 | 31.142 | 1.00 | 14.67 | | O |
| ANISOU | 1454 | O | VAL | F | 143 | 1650 | 1968 | 1954 | 105 | −41 | 335 | O |
| ATOM | 1456 | N | ALA | F | 144 | −9.197 | 42.459 | 29.304 | 1.00 | 14.30 | | N |
| ANISOU | 1456 | N | ALA | F | 144 | 1581 | 2000 | 1849 | −258 | 33 | 7 | N |
| ATOM | 1457 | CA | ALA | F | 144 | −10.644 | 42.392 | 29.196 | 1.00 | 14.71 | | C |
| ANISOU | 1457 | CA | ALA | F | 144 | 1677 | 2018 | 1891 | −89 | −96 | 0 | C |
| ATOM | 1459 | CB | ALA | F | 144 | −11.279 | 43.580 | 29.883 | 1.00 | 17.42 | | C |
| ANISOU | 1459 | CB | ALA | F | 144 | 1848 | 2627 | 2141 | 263 | −176 | −199 | C |
| ATOM | 1463 | C | ALA | F | 144 | −11.088 | 42.333 | 27.756 | 1.00 | 14.29 | | C |
| ANISOU | 1463 | C | ALA | F | 144 | 1699 | 1913 | 1815 | 63 | −72 | 164 | C |
| ATOM | 1464 | O | ALA | F | 144 | −10.357 | 42.738 | 26.855 | 1.00 | 14.98 | | O |
| ANISOU | 1464 | O | ALA | F | 144 | 1857 | 2155 | 1678 | −332 | 5 | 43 | O |
| ATOM | 1466 | N | SER | F | 145 | −12.300 | 41.829 | 27.563 | 1.00 | 14.33 | | N |
| ANISOU | 1466 | N | SER | F | 145 | 1738 | 1834 | 1871 | 153 | −55 | 95 | N |
| ATOM | 1467 | CA | SER | F | 145 | −12.905 | 41.711 | 26.247 | 1.00 | 13.12 | | C |
| ANISOU | 1467 | CA | SER | F | 145 | 1566 | 1818 | 1599 | 93 | 55 | 12 | C |
| ATOM | 1469 | CB | SER | F | 145 | −13.643 | 40.374 | 26.146 | 1.00 | 14.72 | | C |
| ANISOU | 1469 | CB | SER | F | 145 | 1784 | 1935 | 1871 | 34 | −116 | 184 | C |
| ATOM | 1472 | OG | SER | F | 145 | −14.234 | 40.187 | 24.877 | 1.00 | 15.44 | | O |
| ANISOU | 1472 | OG | SER | F | 145 | 2071 | 1912 | 1882 | −60 | −257 | 138 | O |
| ATOM | 1474 | C | SER | F | 145 | −13.870 | 42.874 | 26.053 | 1.00 | 13.64 | | C |
| ANISOU | 1474 | C | SER | F | 145 | 1869 | 1741 | 1570 | 49 | −41 | −59 | C |
| ATOM | 1475 | O | SER | F | 145 | −14.926 | 42.915 | 26.670 | 1.00 | 15.00 | | O |
| ANISOU | 1475 | O | SER | F | 145 | 1792 | 1900 | 2004 | 136 | −72 | 293 | O |
| ATOM | 1477 | N | LEU | F | 146 | −13.491 | 43.809 | 25.188 | 1.00 | 13.65 | | N |
| ANISOU | 1477 | N | LEU | F | 146 | 1629 | 1688 | 1868 | 87 | 180 | 84 | N |
| ATOM | 1478 | CA | LEU | F | 146 | −14.249 | 45.029 | 24.989 | 1.00 | 12.77 | | C |
| ANISOU | 1478 | CA | LEU | F | 146 | 1593 | 1696 | 1561 | 102 | −66 | 9 | C |
| ATOM | 1480 | CB | LEU | F | 146 | −13.355 | 46.264 | 25.132 | 1.00 | 13.54 | | C |
| ANISOU | 1480 | CB | LEU | F | 146 | 1786 | 1624 | 1733 | 175 | −207 | −13 | C |
| ATOM | 1483 | CG | LEU | F | 146 | −12.538 | 46.359 | 26.429 | 1.00 | 14.27 | | C |
| ANISOU | 1483 | CG | LEU | F | 146 | 1684 | 1857 | 1881 | 136 | −235 | 47 | C |
| ATOM | 1485 | CD1 | LEU | F | 146 | −11.796 | 47.662 | 26.454 | 1.00 | 16.12 | | C |
| ANISOU | 1485 | CD1 | LEU | F | 146 | 2009 | 2116 | 1997 | −182 | 1 | −259 | C |
| ATOM | 1489 | CD2 | LEU | F | 146 | −13.405 | 46.243 | 27.662 | 1.00 | 16.12 | | C |
| ANISOU | 1489 | CD2 | LEU | F | 146 | 2010 | 2106 | 2007 | 84 | −173 | 32 | C |
| ATOM | 1493 | C | LEU | F | 146 | −14.911 | 45.015 | 23.619 | 1.00 | 11.76 | | C |
| ANISOU | 1493 | C | LEU | F | 146 | 1605 | 1484 | 1379 | 82 | −97 | 128 | C |
| ATOM | 1494 | O | LEU | F | 146 | −14.483 | 44.296 | 22.708 | 1.00 | 13.59 | | O |
| ANISOU | 1494 | O | LEU | F | 146 | 1777 | 1813 | 1573 | 229 | −46 | 23 | O |
| ATOM | 1496 | N | ALA | F | 147 | −15.966 | 45.811 | 23.496 | 1.00 | 12.27 | | N |
| ANISOU | 1496 | N | ALA | F | 147 | 1728 | 1551 | 1382 | 46 | −21 | 131 | N |
| ATOM | 1497 | CA | ALA | F | 147 | −16.751 | 45.868 | 22.273 | 1.00 | 12.45 | | C |
| ANISOU | 1497 | CA | ALA | F | 147 | 1658 | 1540 | 1531 | 89 | −130 | −37 | C |
| ATOM | 1499 | CB | ALA | F | 147 | −17.961 | 44.958 | 22.375 | 1.00 | 14.04 | | C |
| ANISOU | 1499 | CB | ALA | F | 147 | 1903 | 1898 | 1532 | 4 | −320 | 131 | C |
| ATOM | 1503 | C | ALA | F | 147 | −17.204 | 47.288 | 22.018 | 1.00 | 13.71 | | C |
| ANISOU | 1503 | C | ALA | F | 147 | 1922 | 1649 | 1638 | 77 | −57 | 121 | C |
| ATOM | 1504 | O | ALA | F | 147 | −17.252 | 48.119 | 22.930 | 1.00 | 13.17 | | O |
| ANISOU | 1504 | O | ALA | F | 147 | 2001 | 1366 | 1636 | 151 | −225 | 131 | O |
| ATOM | 1506 | N | PHE | F | 148 | −17.540 | 47.554 | 20.763 | 1.00 | 15.34 | | N |
| ANISOU | 1506 | N | PHE | F | 148 | 2287 | 1854 | 1687 | 42 | −122 | 57 | N |
| ATOM | 1507 | CA | PHE | F | 148 | −18.155 | 48.816 | 20.378 | 1.00 | 14.72 | | C |
| ANISOU | 1507 | CA | PHE | F | 148 | 2143 | 1621 | 1828 | −27 | −159 | 168 | C |
| ATOM | 1509 | CB | PHE | F | 148 | −18.754 | 48.676 | 18.976 | 1.00 | 18.27 | | C |
| ANISOU | 1509 | CB | PHE | F | 148 | 2409 | 2345 | 2185 | −63 | −164 | 160 | C |
| ATOM | 1512 | CG | PHE | F | 148 | −19.523 | 49.885 | 18.500 | 1.00 | 18.22 | | C |
| ANISOU | 1512 | CG | PHE | F | 148 | 2352 | 2215 | 2354 | −15 | −100 | 297 | C |
| ATOM | 1513 | CD1 | PHE | F | 148 | −18.985 | 51.166 | 18.602 | 1.00 | 19.45 | | C |
| ANISOU | 1513 | CD1 | PHE | F | 148 | 2628 | 2239 | 2523 | 93 | −85 | 225 | C |
| ATOM | 1515 | CE1 | PHE | F | 148 | −19.696 | 52.272 | 18.154 | 1.00 | 20.71 | | C |
| ANISOU | 1515 | CE1 | PHE | F | 148 | 2676 | 2495 | 2698 | 3 | −39 | 201 | C |
| ATOM | 1517 | CZ | PHE | F | 148 | −20.940 | 52.103 | 17.594 | 1.00 | 20.99 | | C |
| ANISOU | 1517 | CZ | PHE | F | 148 | 2766 | 2479 | 2729 | −16 | −87 | 201 | C |
| ATOM | 1519 | CE2 | PHE | F | 148 | −21.482 | 50.830 | 17.473 | 1.00 | 19.63 | | C |
| ANISOU | 1519 | CE2 | PHE | F | 148 | 2405 | 2577 | 2475 | 30 | −273 | 259 | C |
| ATOM | 1521 | CD2 | PHE | F | 148 | −20.775 | 49.732 | 17.919 | 1.00 | 20.60 | | C |
| ANISOU | 1521 | CD2 | PHE | F | 148 | 2659 | 2513 | 2654 | −142 | −23 | 135 | C |
| ATOM | 1523 | C | PHE | F | 148 | −19.224 | 49.216 | 21.385 | 1.00 | 15.21 | | C |
| ANISOU | 1523 | C | PHE | F | 148 | 1955 | 1934 | 1891 | 141 | −261 | 334 | C |
| ATOM | 1524 | O | PHE | F | 148 | −20.058 | 48.397 | 21.775 | 1.00 | 14.51 | | O |
| ANISOU | 1524 | O | PHE | F | 148 | 2027 | 1665 | 1820 | −73 | −400 | 118 | O |
| ATOM | 1526 | N | LYS | F | 149 | −19.140 | 50.475 | 21.823 | 1.00 | 16.65 | | N |
| ANISOU | 1526 | N | LYS | F | 149 | 2472 | 1908 | 1943 | −84 | −160 | −23 | N |
| ATOM | 1527 | CA | LYS | F | 149 | −20.077 | 51.130 | 22.756 | 1.00 | 18.14 | | C |
| ANISOU | 1527 | CA | LYS | F | 149 | 2651 | 2070 | 2169 | 144 | 19 | −20 | C |
| ATOM | 1529 | CB | LYS | F | 149 | −21.542 | 50.729 | 22.530 | 1.00 | 19.99 | | C |
| ANISOU | 1529 | CB | LYS | F | 149 | 2782 | 2384 | 2430 | 162 | −16 | −177 | C |
| ATOM | 1532 | CG | LYS | F | 149 | −22.063 | 51.128 | 21.155 | 1.00 | 23.10 | | C |
| ANISOU | 1532 | CG | LYS | F | 149 | 3158 | 2833 | 2786 | 136 | −79 | −18 | C |
| ATOM | 1535 | CD | LYS | F | 149 | −23.566 | 51.042 | 21.094 | 1.00 | 24.33 | | C |
| ANISOU | 1535 | CD | LYS | F | 149 | 3169 | 3101 | 2972 | 94 | −1 | −38 | C |
| ATOM | 1538 | CE | LYS | F | 149 | −24.111 | 51.559 | 19.774 | 1.00 | 25.06 | | C |
| ANISOU | 1538 | CE | LYS | F | 149 | 3276 | 3269 | 2978 | −29 | −104 | −14 | C |
| ATOM | 1541 | NZ | LYS | F | 149 | −25.591 | 51.506 | 19.775 | 1.00 | 26.84 | | N |
| ANISOU | 1541 | NZ | LYS | F | 149 | 3385 | 3562 | 3250 | 131 | −42 | 65 | N |
| ATOM | 1545 | C | LYS | F | 149 | −19.690 | 51.009 | 24.230 | 1.00 | 16.78 | | C |
| ANISOU | 1545 | C | LYS | F | 149 | 2139 | 2052 | 2184 | 139 | −99 | 0 | C |
| ATOM | 1546 | O | LYS | F | 149 | −20.267 | 51.696 | 25.084 | 1.00 | 19.16 | | O |
| ANISOU | 1546 | O | LYS | F | 149 | 2633 | 2405 | 2241 | 135 | −290 | −364 | O |
| ATOM | 1548 | N | ASP | F | 150 | −18.700 | 50.167 | 24.527 | 1.00 | 14.59 | | N |
| ANISOU | 1548 | N | ASP | F | 150 | 2024 | 1760 | 1757 | 41 | −97 | −203 | N |
| ATOM | 1549 | CA | ASP | F | 150 | −18.148 | 50.100 | 25.871 | 1.00 | 14.96 | | C |
| ANISOU | 1549 | CA | ASP | F | 150 | 2029 | 1749 | 1904 | −96 | −54 | −115 | C |
| ATOM | 1551 | CB | ASP | F | 150 | −17.105 | 48.985 | 26.004 | 1.00 | 14.34 | | C |
| ANISOU | 1551 | CB | ASP | F | 150 | 1849 | 1777 | 1823 | −96 | −123 | −82 | C |
| ATOM | 1554 | CG | ASP | F | 150 | −17.710 | 47.589 | 26.119 | 1.00 | 14.19 | | C |
| ANISOU | 1554 | CG | ASP | F | 150 | 1777 | 1800 | 1813 | 101 | 63 | −103 | C |
| ATOM | 1555 | OD1 | ASP | F | 150 | −18.947 | 47.432 | 26.300 | 1.00 | 15.63 | | O |
| ANISOU | 1555 | OD1 | ASP | F | 150 | 2038 | 1933 | 1965 | −135 | −2 | −160 | O |
| ATOM | 1556 | OD2 | ASP | F | 150 | −16.915 | 46.618 | 26.035 | 1.00 | 15.23 | | O |
| ANISOU | 1556 | OD2 | ASP | F | 150 | 2036 | 2027 | 1723 | 177 | −21 | −250 | O |
| ATOM | 1557 | C | ASP | F | 150 | −17.480 | 51.442 | 26.179 | 1.00 | 14.31 | | C |
| ANISOU | 1557 | C | ASP | F | 150 | 2084 | 1499 | 1851 | −154 | 267 | 19 | C |
| ATOM | 1558 | O | ASP | F | 150 | −16.922 | 52.085 | 25.288 | 1.00 | 15.78 | | O |
| ANISOU | 1558 | O | ASP | F | 150 | 2283 | 1751 | 1959 | −302 | 131 | −245 | O |
| ATOM | 1560 | N | LYS | F | 151 | −17.551 | 51.838 | 27.446 | 1.00 | 15.98 | | N |
| ANISOU | 1560 | N | LYS | F | 151 | 2167 | 1924 | 1981 | −323 | −14 | −130 | N |
| ATOM | 1561 | CA | LYS | F | 151 | −16.925 | 53.057 | 27.950 | 1.00 | 16.95 | | C |
| ANISOU | 1561 | CA | LYS | F | 151 | 2251 | 2027 | 2160 | −277 | −11 | −185 | C |
| ATOM | 1563 | CB | LYS | F | 151 | −17.975 | 54.052 | 28.439 | 1.00 | 21.98 | | C |
| ANISOU | 1563 | CB | LYS | F | 151 | 2958 | 2424 | 2968 | −142 | 59 | −222 | C |
| ATOM | 1566 | CG | LYS | F | 151 | −19.023 | 54.433 | 27.423 | 1.00 | 26.42 | | C |
| ANISOU | 1566 | CG | LYS | F | 151 | 3483 | 3123 | 3430 | 17 | 16 | −147 | C |
| ATOM | 1569 | CD | LYS | F | 151 | −18.438 | 55.159 | 26.236 | 1.00 | 27.37 | | C |
| ANISOU | 1569 | CD | LYS | F | 151 | 3482 | 3342 | 3573 | −86 | 13 | −64 | C |
| ATOM | 1572 | CE | LYS | F | 151 | −19.526 | 55.875 | 25.451 | 1.00 | 29.75 | | C |
| ANISOU | 1572 | CE | LYS | F | 151 | 3941 | 3676 | 3686 | −84 | −57 | 113 | C |
| ATOM | 1575 | NZ | LYS | F | 151 | −19.113 | 56.133 | 24.045 | 1.00 | 33.11 | | N |
| ANISOU | 1575 | NZ | LYS | F | 151 | 4424 | 4189 | 3966 | −82 | 165 | 53 | N |
| ATOM | 1579 | C | LYS | F | 151 | −16.044 | 52.646 | 29.118 | 1.00 | 16.77 | | C |
| ANISOU | 1579 | C | LYS | F | 151 | 2090 | 2122 | 2159 | −129 | 10 | −277 | C |
| ATOM | 1580 | O | LYS | F | 151 | −16.522 | 52.036 | 30.079 | 1.00 | 20.57 | | O |
| ANISOU | 1580 | O | LYS | F | 151 | 2455 | 2875 | 2482 | −388 | −4 | −75 | O |
| ATOM | 1582 | N | VAL | F | 152 | −14.757 | 52.961 | 29.037 | 1.00 | 14.98 | | N |
| ANISOU | 1582 | N | VAL | F | 152 | 1942 | 1868 | 1878 | −113 | −55 | −144 | N |
| ATOM | 1583 | CA | VAL | F | 152 | −13.842 | 52.651 | 30.123 | 1.00 | 16.07 | | C |
| ANISOU | 1583 | CA | VAL | F | 152 | 2182 | 2084 | 1839 | −107 | −110 | −193 | C |
| ATOM | 1585 | CB | VAL | F | 152 | −12.464 | 52.195 | 29.612 | 1.00 | 15.99 | | C |
| ANISOU | 1585 | CB | VAL | F | 152 | 2210 | 2014 | 1850 | −50 | −34 | −43 | C |
| ATOM | 1587 | CG1 | VAL | F | 152 | −11.540 | 51.877 | 30.784 | 1.00 | 17.64 | | C |
| ANISOU | 1587 | CG1 | VAL | F | 152 | 2311 | 2240 | 2151 | 34 | −6 | −186 | C |
| ATOM | 1591 | CG2 | VAL | F | 152 | −12.616 | 50.982 | 28.704 | 1.00 | 17.84 | | C |
| ANISOU | 1591 | CG2 | VAL | F | 152 | 2263 | 2450 | 2063 | 139 | −138 | −137 | C |
| ATOM | 1595 | C | VAL | F | 152 | −13.713 | 53.907 | 30.976 | 1.00 | 15.13 | | C |
| ANISOU | 1595 | C | VAL | F | 152 | 2073 | 1854 | 1822 | −109 | −11 | −80 | C |
| ATOM | 1596 | O | VAL | F | 152 | −13.307 | 54.956 | 30.487 | 1.00 | 14.98 | | O |
| ANISOU | 1596 | O | VAL | F | 152 | 2138 | 1834 | 1718 | −109 | 164 | −234 | O |
| ATOM | 1598 | N | TYR | F | 153 | −14.072 | 53.803 | 32.251 | 1.00 | 15.68 | | N |
| ANISOU | 1598 | N | TYR | F | 153 | 2222 | 1825 | 1908 | −217 | −96 | −279 | N |
| ATOM | 1599 | CA | TYR | F | 153 | −13.977 | 54.948 | 33.148 | 1.00 | 15.00 | | C |
| ANISOU | 1599 | CA | TYR | F | 153 | 1923 | 1963 | 1809 | −189 | −28 | −244 | C |
| ATOM | 1601 | CB | TYR | F | 153 | −15.224 | 55.827 | 33.030 | 1.00 | 15.97 | | C |
| ANISOU | 1601 | CB | TYR | F | 153 | 2187 | 1997 | 1880 | −38 | −26 | −214 | C |
| ATOM | 1604 | CG | TYR | F | 153 | −16.523 | 55.180 | 33.432 | 1.00 | 17.16 | | C |
| ANISOU | 1604 | CG | TYR | F | 153 | 2142 | 2249 | 2127 | −15 | −108 | −15 | C |
| ATOM | 1605 | CD1 | TYR | F | 153 | −17.254 | 54.422 | 32.522 | 1.00 | 17.94 | | C |
| ANISOU | 1605 | CD1 | TYR | F | 153 | 2168 | 2350 | 2296 | −125 | −165 | −112 | C |
| ATOM | 1607 | CE1 | TYR | F | 153 | −18.462 | 53.838 | 32.879 | 1.00 | 17.94 | | C |
| ANISOU | 1607 | CE1 | TYR | F | 153 | 2240 | 2410 | 2165 | −143 | −136 | −23 | C |
| ATOM | 1609 | CZ | TYR | F | 153 | −18.960 | 54.012 | 34.156 | 1.00 | 17.93 | | C |
| ANISOU | 1609 | CZ | TYR | F | 153 | 2030 | 2449 | 2332 | −210 | 65 | 187 | C |
| ATOM | 1610 | OH | TYR | F | 153 | −20.164 | 53.423 | 34.503 | 1.00 | 22.57 | | O |
| ANISOU | 1610 | OH | TYR | F | 153 | 2340 | 3155 | 3078 | −259 | −13 | 304 | O |
| ATOM | 1612 | CE2 | TYR | F | 153 | −18.256 | 54.766 | 35.076 | 1.00 | 19.28 | | C |
| ANISOU | 1612 | CE2 | TYR | F | 153 | 2295 | 2660 | 2369 | −82 | 89 | 99 | C |
| ATOM | 1614 | CD2 | TYR | F | 153 | −17.050 | 55.355 | 34.707 | 1.00 | 18.05 | | C |
| ANISOU | 1614 | CD2 | TYR | F | 153 | 2171 | 2410 | 2277 | −78 | −108 | −51 | C |
| ATOM | 1616 | C | TYR | F | 153 | −13.709 | 54.504 | 34.576 | 1.00 | 14.39 | | C |
| ANISOU | 1616 | C | TYR | F | 153 | 1933 | 1688 | 1845 | −71 | −34 | −233 | C |
| ATOM | 1617 | O | TYR | F | 153 | −13.726 | 53.311 | 34.885 | 1.00 | 15.30 | | O |
| ANISOU | 1617 | O | TYR | F | 153 | 2191 | 1828 | 1792 | −15 | −89 | −292 | O |
| ATOM | 1619 | N | LEU | F | 154 | −13.438 | 55.481 | 35.438 | 1.00 | 14.62 | | N |
| ANISOU | 1619 | N | LEU | F | 154 | 2173 | 1661 | 1720 | −72 | −83 | −254 | N |
| ATOM | 1620 | CA | LEU | F | 154 | −12.935 | 55.216 | 36.776 | 1.00 | 14.16 | | C |
| ANISOU | 1620 | CA | LEU | F | 154 | 1897 | 1632 | 1850 | −30 | −92 | −291 | C |
| ATOM | 1622 | CB | LEU | F | 154 | −11.477 | 55.654 | 36.868 | 1.00 | 15.77 | | C |
| ANISOU | 1622 | CB | LEU | F | 154 | 1941 | 1976 | 2072 | −125 | 55 | −20 | C |
| ATOM | 1625 | CG | LEU | F | 154 | −10.485 | 54.926 | 35.958 | 1.00 | 16.54 | | C |
| ANISOU | 1625 | CG | LEU | F | 154 | 1997 | 2029 | 2257 | −100 | −32 | −190 | C |
| ATOM | 1627 | CD1 | LEU | F | 154 | −9.188 | 55.718 | 35.786 | 1.00 | 17.99 | | C |
| ANISOU | 1627 | CD1 | LEU | F | 154 | 2064 | 2485 | 2286 | −21 | 148 | −184 | C |
| ATOM | 1631 | CD2 | LEU | F | 154 | −10.199 | 53.546 | 36.527 | 1.00 | 17.90 | | C |
| ANISOU | 1631 | CD2 | LEU | F | 154 | 2185 | 2087 | 2529 | −48 | 31 | −47 | C |
| ATOM | 1635 | C | LEU | F | 154 | −13.763 | 56.015 | 37.753 | 1.00 | 14.32 | | C |
| ANISOU | 1635 | C | LEU | F | 154 | 1692 | 1931 | 1816 | −90 | 84 | −183 | C |
| ATOM | 1636 | O | LEU | F | 154 | −13.955 | 57.211 | 37.551 | 1.00 | 14.79 | | O |
| ANISOU | 1636 | O | LEU | F | 154 | 1969 | 1804 | 1844 | −70 | −1 | −94 | O |
| ATOM | 1638 | N | THR | F | 155 | −14.266 | 55.361 | 38.795 | 1.00 | 14.61 | | N |
| ANISOU | 1638 | N | THR | F | 155 | 2023 | 1773 | 1755 | 118 | −23 | −203 | N |
| ATOM | 1639 | CA | THR | F | 155 | −14.992 | 56.064 | 39.853 | 1.00 | 16.14 | | C |
| ANISOU | 1639 | CA | THR | F | 155 | 2162 | 1980 | 1990 | 192 | 142 | −103 | C |
| ATOM | 1641 | CB | THR | F | 155 | −16.467 | 55.639 | 39.928 | 1.00 | 17.47 | | C |
| ANISOU | 1641 | CB | THR | F | 155 | 2155 | 2311 | 2173 | 165 | 88 | −150 | C |
| ATOM | 1643 | OG1 | THR | F | 155 | −16.559 | 54.235 | 40.186 | 1.00 | 18.42 | | O |
| ANISOU | 1643 | OG1 | THR | F | 155 | 2302 | 2289 | 2405 | 6 | −13 | −154 | O |
| ATOM | 1645 | CG2 | THR | F | 155 | −17.190 | 55.960 | 38.624 | 1.00 | 20.08 | | C |
| ANISOU | 1645 | CG2 | THR | F | 155 | 2356 | 2854 | 2417 | 138 | −41 | −6 | C |
| ATOM | 1649 | C | THR | F | 155 | −14.332 | 55.835 | 41.200 | 1.00 | 17.71 | | C |
| ANISOU | 1649 | C | THR | F | 155 | 2334 | 2206 | 2188 | 191 | 95 | −78 | C |
| ATOM | 1650 | O | THR | F | 155 | −13.578 | 54.886 | 41.383 | 1.00 | 17.14 | | O |
| ANISOU | 1650 | O | THR | F | 155 | 2510 | 2028 | 1973 | 258 | −227 | −336 | O |
| ATOM | 1652 | N | VAL | F | 156 | −14.639 | 56.726 | 42.134 | 1.00 | 17.18 | | N |
| ANISOU | 1652 | N | VAL | F | 156 | 2513 | 2066 | 1947 | 58 | −64 | −194 | N |
| ATOM | 1653 | CA | VAL | F | 156 | −14.067 | 56.706 | 43.471 | 1.00 | 20.01 | | C |
| ANISOU | 1653 | CA | VAL | F | 156 | 2818 | 2483 | 2300 | 94 | −1 | −69 | C |
| ATOM | 1655 | CB | VAL | F | 156 | −13.322 | 58.034 | 43.781 | 1.00 | 21.87 | | C |
| ANISOU | 1655 | CB | VAL | F | 156 | 2835 | 2825 | 2647 | −44 | 70 | −64 | C |
| ATOM | 1657 | CG1 | VAL | F | 156 | −12.735 | 58.014 | 45.188 | 1.00 | 23.10 | | C |
| ANISOU | 1657 | CG1 | VAL | F | 156 | 3024 | 2856 | 2895 | −38 | −19 | −6 | C |
| ATOM | 1661 | CG2 | VAL | F | 156 | −12.225 | 58.278 | 42.753 | 1.00 | 22.73 | | C |
| ANISOU | 1661 | CG2 | VAL | F | 156 | 2923 | 2989 | 2723 | 77 | 135 | −5 | C |
| ATOM | 1665 | C | VAL | F | 156 | −15.214 | 56.513 | 44.445 | 1.00 | 22.61 | | C |
| ANISOU | 1665 | C | VAL | F | 156 | 3012 | 2820 | 2758 | 72 | 3 | 25 | C |
| ATOM | 1666 | O | VAL | F | 156 | −16.205 | 57.239 | 44.390 | 1.00 | 20.94 | | O |
| ANISOU | 1666 | O | VAL | F | 156 | 2737 | 2941 | 2275 | 214 | 308 | −190 | O |
| ATOM | 1668 | N | ASN | F | 157 | −15.093 | 55.506 | 45.305 | 1.00 | 23.72 | | N |
| ANISOU | 1668 | N | ASN | F | 157 | 3244 | 2895 | 2871 | −71 | −7 | −5 | N |
| ATOM | 1669 | CA | ASN | F | 157 | −16.070 | 55.275 | 46.347 | 1.00 | 23.81 | | C |
| ANISOU | 1669 | CA | ASN | F | 157 | 3171 | 2906 | 2968 | −98 | −16 | 13 | C |
| ATOM | 1671 | CB | ASN | F | 157 | −16.312 | 53.773 | 46.542 | 1.00 | 27.76 | | C |
| ANISOU | 1671 | CB | ASN | F | 157 | 3612 | 3265 | 3669 | −12 | −48 | 62 | C |
| ATOM | 1674 | CG | ASN | F | 157 | −17.725 | 53.465 | 47.014 | 1.00 | 31.77 | | C |
| ANISOU | 1674 | CG | ASN | F | 157 | 3978 | 3960 | 4131 | −61 | 20 | 65 | C |
| ATOM | 1675 | OD1 | ASN | F | 157 | −18.299 | 54.199 | 47.821 | 1.00 | 34.50 | | O |
| ANISOU | 1675 | OD1 | ASN | F | 157 | 4386 | 4267 | 4453 | 0 | 95 | 27 | O |
| ATOM | 1676 | ND2 | ASN | F | 157 | −18.294 | 52.379 | 46.501 | 1.00 | 34.88 | | N |
| ANISOU | 1676 | ND2 | ASN | F | 157 | 4636 | 4068 | 4549 | 9 | −88 | 0 | N |
| ATOM | 1679 | C | ASN | F | 157 | −15.543 | 55.942 | 47.614 | 1.00 | 23.27 | | C |
| ANISOU | 1679 | C | ASN | F | 157 | 3216 | 2884 | 2742 | 26 | −12 | −50 | C |
| ATOM | 1680 | O | ASN | F | 157 | −14.744 | 55.360 | 48.352 | 1.00 | 25.94 | | O |
| ANISOU | 1680 | O | ASN | F | 157 | 3555 | 3123 | 3178 | 132 | −145 | −42 | O |
| ATOM | 1682 | N | ALA | F | 158 | −15.959 | 57.186 | 47.828 | 1.00 | 21.24 | | N |
| ANISOU | 1682 | N | ALA | F | 158 | 2834 | 2610 | 2624 | −101 | −82 | 139 | N |
| ATOM | 1683 | CA | ALA | F | 158 | −15.507 | 57.972 | 48.975 | 1.00 | 20.22 | | C |
| ANISOU | 1683 | CA | ALA | F | 158 | 2540 | 2532 | 2611 | −86 | 35 | 34 | C |
| ATOM | 1685 | CB | ALA | F | 158 | −14.069 | 58.432 | 48.774 | 1.00 | 21.35 | | C |
| ANISOU | 1685 | CB | ALA | F | 158 | 2738 | 2656 | 2718 | 18 | 4 | 48 | C |
| ATOM | 1689 | C | ALA | F | 158 | −16.406 | 59.185 | 49.180 | 1.00 | 19.68 | | C |
| ANISOU | 1689 | C | ALA | F | 158 | 2372 | 2430 | 2674 | −258 | 28 | 190 | C |
| ATOM | 1690 | O | ALA | F | 158 | −17.081 | 59.621 | 48.244 | 1.00 | 19.41 | | O |
| ANISOU | 1690 | O | ALA | F | 158 | 2248 | 2481 | 2647 | −372 | 31 | 311 | O |
| ATOM | 1692 | N | PRO | F | 159 | −16.415 | 59.740 | 50.405 | 1.00 | 19.80 | | N |
| ANISOU | 1692 | N | PRO | F | 159 | 2532 | 2408 | 2581 | −213 | −14 | 106 | N |
| ATOM | 1693 | CA | PRO | F | 159 | −17.074 | 61.025 | 50.630 | 1.00 | 20.70 | | C |
| ANISOU | 1693 | CA | PRO | F | 159 | 2432 | 2673 | 2758 | −9 | 95 | 80 | C |
| ATOM | 1695 | CB | PRO | F | 159 | −16.655 | 61.394 | 52.056 | 1.00 | 21.14 | | C |
| ANISOU | 1695 | CB | PRO | F | 159 | 2693 | 2630 | 2706 | −60 | −5 | 37 | C |
| ATOM | 1698 | CG | PRO | F | 159 | −16.342 | 60.097 | 52.712 | 1.00 | 21.88 | | C |
| ANISOU | 1698 | CG | PRO | F | 159 | 2847 | 2708 | 2755 | −10 | −62 | 7 | C |
| ATOM | 1701 | CD | PRO | F | 159 | −15.824 | 59.192 | 51.639 | 1.00 | 21.49 | | C |
| ANISOU | 1701 | CD | PRO | F | 159 | 2755 | 2675 | 2733 | −79 | −90 | 57 | C |
| ATOM | 1704 | C | PRO | F | 159 | −16.564 | 62.062 | 49.637 | 1.00 | 20.20 | | C |
| ANISOU | 1704 | C | PRO | F | 159 | 2347 | 2509 | 2820 | −60 | 171 | 59 | C |
| ATOM | 1705 | O | PRO | F | 159 | −15.376 | 62.051 | 49.294 | 1.00 | 19.76 | | O |
| ANISOU | 1705 | O | PRO | F | 159 | 2100 | 2556 | 2851 | −136 | 298 | 61 | O |
| ATOM | 1706 | N | ASP | F | 160 | −17.436 | 62.945 | 49.164 | 1.00 | 23.30 | | N |
| ANISOU | 1706 | N | ASP | F | 160 | 2724 | 3078 | 3052 | −104 | 81 | 183 | N |
| ATOM | 1707 | CA | ASP | F | 160 | −17.041 | 63.831 | 48.071 | 1.00 | 23.19 | | C |
| ANISOU | 1707 | CA | ASP | F | 160 | 2839 | 2985 | 2984 | −120 | −29 | 168 | C |
| ATOM | 1709 | CB | ASP | F | 160 | −18.227 | 64.595 | 47.485 | 1.00 | 26.81 | | C |
| ANISOU | 1709 | CB | ASP | F | 160 | 3329 | 3469 | 3385 | −50 | −109 | 199 | C |
| ATOM | 1712 | CG | ASP | F | 160 | −17.985 | 65.002 | 46.042 | 1.00 | 27.83 | | C |
| ANISOU | 1712 | CG | ASP | F | 160 | 3508 | 3637 | 3427 | −13 | −54 | 173 | C |
| ATOM | 1713 | OD1 | ASP | F | 160 | −17.697 | 64.115 | 45.206 | 1.00 | 31.31 | | O |
| ANISOU | 1713 | OD1 | ASP | F | 160 | 3877 | 4357 | 3663 | −60 | 90 | 195 | O |
| ATOM | 1714 | OD2 | ASP | F | 160 | −18.077 | 66.207 | 45.745 | 1.00 | 31.16 | | O |
| ANISOU | 1714 | OD2 | ASP | F | 160 | 4105 | 3836 | 3898 | −271 | −113 | 171 | O |
| ATOM | 1715 | C | ASP | F | 160 | −15.915 | 64.793 | 48.453 | 1.00 | 20.36 | | C |
| ANISOU | 1715 | C | ASP | F | 160 | 2656 | 2601 | 2479 | −51 | −18 | 34 | C |
| ATOM | 1716 | O | ASP | F | 160 | −15.123 | 65.167 | 47.597 | 1.00 | 19.54 | | O |
| ANISOU | 1716 | O | ASP | F | 160 | 2178 | 2879 | 2366 | −29 | 148 | 359 | O |
| ATOM | 1718 | N | THR | F | 161 | −15.817 | 65.177 | 49.723 | 1.00 | 19.03 | | N |
| ANISOU | 1718 | N | THR | F | 161 | 2359 | 2502 | 2367 | −39 | 109 | 261 | N |
| ATOM | 1719 | CA | THR | F | 161 | −14.706 | 66.017 | 50.162 | 1.00 | 18.22 | | C |
| ANISOU | 1719 | CA | THR | F | 161 | 2314 | 2310 | 2299 | 72 | 115 | 133 | C |
| ATOM | 1721 | CB | THR | F | 161 | −14.777 | 66.306 | 51.666 | 1.00 | 18.72 | | C |
| ANISOU | 1721 | CB | THR | F | 161 | 2406 | 2438 | 2268 | 42 | 100 | −2 | C |
| ATOM | 1723 | OG1 | THR | F | 161 | −16.040 | 66.906 | 51.971 | 1.00 | 22.22 | | O |
| ANISOU | 1723 | OG1 | THR | F | 161 | 2621 | 3053 | 2767 | 249 | 200 | 172 | O |
| ATOM | 1725 | CG2 | THR | F | 161 | −13.655 | 67.248 | 52.094 | 1.00 | 19.20 | | C |
| ANISOU | 1725 | CG2 | THR | F | 161 | 2429 | 2516 | 2349 | 11 | 50 | 57 | C |
| ATOM | 1729 | C | THR | F | 161 | −13.364 | 65.359 | 49.847 | 1.00 | 16.95 | | C |
| ANISOU | 1729 | C | THR | F | 161 | 2020 | 2066 | 2353 | −39 | 142 | 286 | C |
| ATOM | 1730 | O | THR | F | 161 | −12.447 | 66.008 | 49.339 | 1.00 | 13.90 | | O |
| ANISOU | 1730 | O | THR | F | 161 | 1604 | 1620 | 2057 | −200 | −65 | 450 | O |
| ATOM | 1732 | N | LEU | F | 162 | −13.252 | 64.067 | 50.141 | 1.00 | 15.21 | | N |
| ANISOU | 1732 | N | LEU | F | 162 | 1861 | 1915 | 2000 | −90 | 262 | 254 | N |
| ATOM | 1733 | CA | LEU | F | 162 | −12.021 | 63.342 | 49.847 | 1.00 | 16.41 | | C |
| ANISOU | 1733 | CA | LEU | F | 162 | 2005 | 2037 | 2192 | −45 | 25 | 127 | C |
| ATOM | 1735 | CB | LEU | F | 162 | −12.078 | 61.912 | 50.392 | 1.00 | 18.52 | | C |
| ANISOU | 1735 | CB | LEU | F | 162 | 2341 | 2134 | 2561 | −51 | −16 | 124 | C |
| ATOM | 1738 | CG | LEU | F | 162 | −12.179 | 61.779 | 51.917 | 1.00 | 23.62 | | C |
| ANISOU | 1738 | CG | LEU | F | 162 | 2934 | 2981 | 3057 | 30 | 33 | 74 | C |
| ATOM | 1740 | CD1 | LEU | F | 162 | −12.185 | 60.307 | 52.320 | 1.00 | 26.31 | | C |
| ANISOU | 1740 | CD1 | LEU | F | 162 | 3262 | 3221 | 3514 | 1 | 12 | 237 | C |
| ATOM | 1744 | CD2 | LEU | F | 162 | −11.051 | 62.520 | 52.632 | 1.00 | 25.26 | | C |
| ANISOU | 1744 | CD2 | LEU | F | 162 | 3127 | 3138 | 3330 | −3 | −58 | 128 | C |
| ATOM | 1748 | C | LEU | F | 162 | −11.765 | 63.338 | 48.345 | 1.00 | 14.91 | | C |
| ANISOU | 1748 | C | LEU | F | 162 | 1716 | 1965 | 1983 | −43 | −171 | 414 | C |
| ATOM | 1749 | O | LEU | F | 162 | −10.637 | 63.550 | 47.908 | 1.00 | 15.58 | | O |
| ANISOU | 1749 | O | LEU | F | 162 | 1511 | 2353 | 2052 | −43 | −47 | 570 | O |
| ATOM | 1751 | N | CYS | F | 163 | −12.814 | 63.124 | 47.555 | 1.00 | 14.01 | | N |
| ANISOU | 1751 | N | CYS | F | 163 | 1815 | 1514 | 1993 | −105 | −61 | 244 | N |
| ATOM | 1752 | CA | CYS | F | 163 | −12.685 | 63.153 | 46.100 | 1.00 | 13.84 | | C |
| ANISOU | 1752 | CA | CYS | F | 163 | 1847 | 1627 | 1785 | 0 | 31 | 19 | C |
| ATOM | 1754 | CB | CYS | F | 163 | −14.008 | 62.795 | 45.417 | 1.00 | 15.00 | | C |
| ANISOU | 1754 | CB | CYS | F | 163 | 1885 | 1604 | 2209 | −238 | 56 | 335 | C |
| ATOM | 1757 | SG | CYS | F | 163 | −14.100 | 63.166 | 43.631 | 1.00 | 19.15 | | S |
| ANISOU | 1757 | SG | CYS | F | 163 | 2439 | 2478 | 2358 | −273 | −194 | 179 | S |
| ATOM | 1759 | C | CYS | F | 163 | −12.168 | 64.493 | 45.592 | 1.00 | 13.67 | | C |
| ANISOU | 1759 | C | CYS | F | 163 | 1735 | 1808 | 1650 | 64 | 65 | 35 | C |
| ATOM | 1760 | O | CYS | F | 163 | −11.195 | 64.537 | 44.840 | 1.00 | 11.70 | | O |
| ANISOU | 1760 | O | CYS | F | 163 | 1551 | 1539 | 1353 | −94 | 158 | 165 | O |
| ATOM | 1762 | N | GLU | F | 164 | −12.792 | 65.586 | 46.024 | 1.00 | 11.33 | | N |
| ANISOU | 1762 | N | GLU | F | 164 | 1200 | 1402 | 1701 | −73 | −35 | 117 | N |
| ATOM | 1763 | CA | GLU | F | 164 | −12.432 | 66.907 | 45.497 | 1.00 | 13.80 | | C |
| ANISOU | 1763 | CA | GLU | F | 164 | 1618 | 1729 | 1895 | −2 | 0 | 115 | C |
| ATOM | 1765 | CB | GLU | F | 164 | −13.418 | 67.978 | 45.966 | 1.00 | 14.55 | | C |
| ANISOU | 1765 | CB | GLU | F | 164 | 1559 | 1827 | 2142 | 45 | −163 | 130 | C |
| ATOM | 1768 | CG | GLU | F | 164 | −14.897 | 67.690 | 45.658 | 1.00 | 18.73 | | C |
| ANISOU | 1768 | CG | GLU | F | 164 | 1945 | 2605 | 2566 | 58 | −126 | 143 | C |
| ATOM | 1771 | CD | GLU | F | 164 | −15.208 | 67.549 | 44.188 | 1.00 | 20.53 | | C |
| ANISOU | 1771 | CD | GLU | F | 164 | 2346 | 2719 | 2735 | 188 | −14 | 128 | C |
| ATOM | 1772 | OE1 | GLU | F | 164 | −14.486 | 68.140 | 43.362 | 1.00 | 24.39 | | O |
| ANISOU | 1772 | OE1 | GLU | F | 164 | 2497 | 3728 | 3041 | 223 | −51 | 460 | O |
| ATOM | 1773 | OE2 | GLU | F | 164 | −16.195 | 66.851 | 43.861 | 1.00 | 20.87 | | O |
| ANISOU | 1773 | OE2 | GLU | F | 164 | 2599 | 2872 | 2460 | 182 | −161 | −161 | O |
| ATOM | 1774 | C | GLU | F | 164 | −11.004 | 67.303 | 45.862 | 1.00 | 13.08 | | C |
| ANISOU | 1774 | C | GLU | F | 164 | 1606 | 1632 | 1732 | 43 | −68 | 59 | C |
| ATOM | 1775 | O | GLU | F | 164 | −10.359 | 68.040 | 45.119 | 1.00 | 12.00 | | O |
| ANISOU | 1775 | O | GLU | F | 164 | 1514 | 1878 | 1164 | −6 | −181 | 80 | O |
| ATOM | 1777 | N | HIS | F | 165 | −10.513 | 66.806 | 46.996 | 1.00 | 11.47 | | N |
| ANISOU | 1777 | N | HIS | F | 165 | 1305 | 1392 | 1660 | −97 | 137 | 328 | N |
| ATOM | 1778 | CA | HIS | F | 165 | −9.159 | 67.113 | 47.467 | 1.00 | 12.32 | | C |
| ANISOU | 1778 | CA | HIS | F | 165 | 1426 | 1566 | 1686 | 47 | −91 | 258 | C |
| ATOM | 1780 | CB | HIS | F | 165 | −9.138 | 67.188 | 48.989 | 1.00 | 12.12 | | C |
| ANISOU | 1780 | CB | HIS | F | 165 | 1504 | 1367 | 1733 | 0 | 21 | 381 | C |
| ATOM | 1783 | CG | HIS | F | 165 | −9.621 | 68.495 | 49.513 | 1.00 | 9.75 | | C |
| ANISOU | 1783 | CG | HIS | F | 165 | 1249 | 1295 | 1160 | 171 | 65 | 315 | C |
| ATOM | 1784 | ND1 | HIS | F | 165 | −10.939 | 68.731 | 49.830 | 1.00 | 12.58 | | N |
| ANISOU | 1784 | ND1 | HIS | F | 165 | 1758 | 1386 | 1634 | −117 | −123 | 753 | N |
| ATOM | 1786 | CE1 | HIS | F | 165 | −11.063 | 69.980 | 50.245 | 1.00 | 7.88 | | C |
| ANISOU | 1786 | CE1 | HIS | F | 165 | 1105 | 874 | 1013 | 36 | −124 | 324 | C |
| ATOM | 1788 | NE2 | HIS | F | 165 | −9.878 | 70.558 | 50.203 | 1.00 | 12.14 | | N |
| ANISOU | 1788 | NE2 | HIS | F | 165 | 1793 | 1496 | 1321 | 347 | 99 | 647 | N |
| ATOM | 1790 | CD2 | HIS | F | 165 | −8.959 | 69.653 | 49.743 | 1.00 | 7.82 | | C |
| ANISOU | 1790 | CD2 | HIS | F | 165 | 657 | 1264 | 1047 | −419 | −75 | 559 | C |
| ATOM | 1792 | C | HIS | F | 165 | −8.082 | 66.139 | 46.995 | 1.00 | 13.20 | | C |
| ANISOU | 1792 | C | HIS | F | 165 | 1591 | 1760 | 1663 | 49 | −130 | 58 | C |
| ATOM | 1793 | O | HIS | F | 165 | −6.903 | 66.417 | 47.165 | 1.00 | 15.07 | | O |
| ANISOU | 1793 | O | HIS | F | 165 | 1652 | 1878 | 2195 | 166 | −183 | −1 | O |
| ATOM | 1795 | N | LEU | F | 166 | −8.481 | 65.029 | 46.374 | 1.00 | 11.52 | | N |
| ANISOU | 1795 | N | LEU | F | 166 | 1211 | 1497 | 1668 | 91 | 230 | 121 | N |
| ATOM | 1796 | CA | LEU | F | 166 | −7.541 | 63.974 | 46.016 | 1.00 | 12.78 | | C |
| ANISOU | 1796 | CA | LEU | F | 166 | 1597 | 1666 | 1590 | 138 | 52 | 110 | C |
| ATOM | 1798 | CB | LEU | F | 166 | −8.290 | 62.804 | 45.382 | 1.00 | 14.44 | | C |
| ANISOU | 1798 | CB | LEU | F | 166 | 1777 | 1764 | 1946 | 120 | 71 | −13 | C |
| ATOM | 1801 | CG | LEU | F | 166 | −7.469 | 61.588 | 44.947 | 1.00 | 15.07 | | C |
| ANISOU | 1801 | CG | LEU | F | 166 | 1990 | 1874 | 1860 | 173 | 122 | 33 | C |
| ATOM | 1803 | CD1 | LEU | F | 166 | −6.897 | 60.873 | 46.154 | 1.00 | 16.42 | | C |
| ANISOU | 1803 | CD1 | LEU | F | 166 | 2374 | 1484 | 2379 | −11 | −60 | 6 | C |
| ATOM | 1807 | CD2 | LEU | F | 166 | −8.340 | 60.652 | 44.141 | 1.00 | 17.07 | | C |
| ANISOU | 1807 | CD2 | LEU | F | 166 | 2248 | 1863 | 2375 | 135 | 24 | −45 | C |
| ATOM | 1811 | C | LEU | F | 166 | −6.477 | 64.472 | 45.049 | 1.00 | 13.67 | | C |
| ANISOU | 1811 | C | LEU | F | 166 | 1790 | 1544 | 1857 | −65 | −29 | 122 | C |
| ATOM | 1812 | O | LEU | F | 166 | −6.800 | 64.994 | 43.987 | 1.00 | 15.26 | | O |
| ANISOU | 1812 | O | LEU | F | 166 | 1777 | 2154 | 1864 | 46 | 27 | 137 | O |
| ATOM | 1814 | N | GLN | F | 167 | −5.212 | 64.310 | 45.441 | 1.00 | 13.19 | | N |
| ANISOU | 1814 | N | GLN | F | 167 | 1565 | 1676 | 1771 | 190 | 89 | 208 | N |
| ATOM | 1815 | CA | GLN | F | 167 | −4.076 | 64.572 | 44.569 | 1.00 | 13.66 | | C |
| ANISOU | 1815 | CA | GLN | F | 167 | 1752 | 1765 | 1672 | 65 | 111 | 123 | C |
| ATOM | 1817 | CB | GLN | F | 167 | −2.905 | 65.188 | 45.344 | 1.00 | 14.94 | | C |
| ANISOU | 1817 | CB | GLN | F | 167 | 1755 | 1930 | 1991 | −50 | 205 | 41 | C |
| ATOM | 1820 | CG | GLN | F | 167 | −1.640 | 65.423 | 44.507 | 1.00 | 15.01 | | C |
| ANISOU | 1820 | CG | GLN | F | 167 | 1914 | 1795 | 1991 | −63 | 127 | 136 | C |
| ATOM | 1823 | CD | GLN | F | 167 | −1.854 | 66.416 | 43.368 | 1.00 | 17.40 | | C |
| ANISOU | 1823 | CD | GLN | F | 167 | 2242 | 2237 | 2129 | −51 | −20 | 58 | C |
| ATOM | 1824 | OE1 | GLN | F | 167 | −2.736 | 67.269 | 43.431 | 1.00 | 20.15 | | O |
| ANISOU | 1824 | OE1 | GLN | F | 167 | 2162 | 2526 | 2969 | −178 | −175 | 569 | O |
| ATOM | 1825 | NE2 | GLN | F | 167 | −1.028 | 66.317 | 42.335 | 1.00 | 19.20 | | N |
| ANISOU | 1825 | NE2 | GLN | F | 167 | 2496 | 2550 | 2249 | −151 | 203 | −22 | N |
| ATOM | 1828 | C | GLN | F | 167 | −3.627 | 63.267 | 43.927 | 1.00 | 13.05 | | C |
| ANISOU | 1828 | C | GLN | F | 167 | 1625 | 1516 | 1817 | 153 | 124 | 120 | C |
| ATOM | 1829 | O | GLN | F | 167 | −3.421 | 62.269 | 44.624 | 1.00 | 13.76 | | O |
| ANISOU | 1829 | O | GLN | F | 167 | 1721 | 1772 | 1736 | 260 | 85 | 260 | O |
| ATOM | 1831 | N | ILE | F | 168 | −3.468 | 63.291 | 42.607 | 1.00 | 14.87 | | N |
| ANISOU | 1831 | N | ILE | F | 168 | 1836 | 1688 | 2124 | 55 | 121 | 175 | N |
| ATOM | 1832 | CA | ILE | F | 168 | −2.938 | 62.170 | 41.853 | 1.00 | 14.23 | | C |
| ANISOU | 1832 | CA | ILE | F | 168 | 1936 | 1779 | 1689 | −47 | 176 | 99 | C |
| ATOM | 1834 | CB | ILE | F | 168 | −3.972 | 61.644 | 40.840 | 1.00 | 15.94 | | C |
| ANISOU | 1834 | CB | ILE | F | 168 | 2066 | 2020 | 1969 | 0 | 136 | 39 | C |
| ATOM | 1836 | CG1 | ILE | F | 168 | −5.324 | 61.437 | 41.534 | 1.00 | 16.59 | | C |
| ANISOU | 1836 | CG1 | ILE | F | 168 | 1944 | 2172 | 2186 | −260 | 41 | 47 | C |
| ATOM | 1839 | CD1 | ILE | F | 168 | −6.452 | 61.063 | 40.606 | 1.00 | 18.38 | | C |
| ANISOU | 1839 | CD1 | ILE | F | 168 | 2182 | 2492 | 2309 | −183 | 45 | 51 | C |
| ATOM | 1843 | CG2 | ILE | F | 168 | −3.478 | 60.345 | 40.195 | 1.00 | 17.40 | | C |
| ANISOU | 1843 | CG2 | ILE | F | 168 | 2314 | 2265 | 2031 | −177 | 266 | −129 | C |
| ATOM | 1847 | C | ILE | F | 168 | −1.694 | 62.638 | 41.110 | 1.00 | 15.01 | | C |
| ANISOU | 1847 | C | ILE | F | 168 | 1943 | 1926 | 1833 | −57 | 118 | −13 | C |
| ATOM | 1848 | O | ILE | F | 168 | −1.783 | 63.526 | 40.258 | 1.00 | 16.28 | | O |
| ANISOU | 1848 | O | ILE | F | 168 | 2220 | 2457 | 1506 | 144 | 387 | 94 | O |
| ATOM | 1850 | N | ASN | F | 169 | −0.545 | 62.066 | 41.464 | 1.00 | 15.48 | | N |
| ANISOU | 1850 | N | ASN | F | 169 | 2082 | 1983 | 1816 | −49 | 235 | 10 | N |
| ATOM | 1851 | CA | ASN | F | 169 | 0.721 | 62.367 | 40.795 | 1.00 | 16.64 | | C |
| ANISOU | 1851 | CA | ASN | F | 169 | 2016 | 2220 | 2084 | 21 | 24 | 152 | C |
| ATOM | 1853 | CB | ASN | F | 169 | 1.901 | 62.353 | 41.779 | 1.00 | 19.99 | | C |
| ANISOU | 1853 | CB | ASN | F | 169 | 2434 | 2650 | 2508 | −129 | −100 | −126 | C |
| ATOM | 1856 | CG | ASN | F | 169 | 1.999 | 63.605 | 42.611 | 1.00 | 23.08 | | C |
| ANISOU | 1856 | CG | ASN | F | 169 | 2933 | 2871 | 2963 | −34 | 71 | 13 | C |
| ATOM | 1857 | OD1 | ASN | F | 169 | 1.207 | 64.538 | 42.473 | 1.00 | 24.26 | | O |
| ANISOU | 1857 | OD1 | ASN | F | 169 | 3101 | 2780 | 3336 | −6 | 286 | −33 | O |
| ATOM | 1858 | ND2 | ASN | F | 169 | 2.993 | 63.632 | 43.485 | 1.00 | 26.89 | | N |
| ANISOU | 1858 | ND2 | ASN | F | 169 | 3415 | 3687 | 3112 | −255 | −114 | 70 | N |
| ATOM | 1861 | C | ASN | F | 169 | 0.990 | 61.329 | 39.730 | 1.00 | 16.40 | | C |
| ANISOU | 1861 | C | ASN | F | 169 | 1896 | 2179 | 2155 | −119 | 23 | 46 | C |
| ATOM | 1862 | O | ASN | F | 169 | 0.919 | 60.136 | 40.009 | 1.00 | 16.84 | | O |
| ANISOU | 1862 | O | ASN | F | 169 | 1903 | 2173 | 2320 | 45 | 78 | −183 | O |
| ATOM | 1864 | N | ASP | F | 170 | 1.316 | 61.796 | 38.529 | 1.00 | 19.57 | | N |
| ANISOU | 1864 | N | ASP | F | 170 | 2391 | 2553 | 2491 | −136 | −12 | −24 | N |
| ATOM | 1865 | CA | ASP | F | 170 | 1.746 | 60.936 | 37.438 | 1.00 | 20.03 | | C |
| ANISOU | 1865 | CA | ASP | F | 170 | 2571 | 2590 | 2447 | −161 | 67 | −40 | C |
| ATOM | 1867 | CB | ASP | F | 170 | 3.135 | 60.353 | 37.744 | 1.00 | 23.81 | | C |
| ANISOU | 1867 | CB | ASP | F | 170 | 2990 | 3061 | 2993 | 81 | 41 | −172 | C |
| ATOM | 1870 | CG | ASP | F | 170 | 4.159 | 61.422 | 38.117 | 1.00 | 31.88 | | C |
| ANISOU | 1870 | CG | ASP | F | 170 | 3865 | 4131 | 4115 | −49 | 33 | 50 | C |
| ATOM | 1871 | OD1 | ASP | F | 170 | 4.171 | 62.495 | 37.476 | 1.00 | 35.54 | | O |
| ANISOU | 1871 | OD1 | ASP | F | 170 | 4337 | 4358 | 4807 | −35 | 238 | 185 | O |
| ATOM | 1872 | OD2 | ASP | F | 170 | 4.961 | 61.190 | 39.048 | 1.00 | 36.73 | | O |
| ANISOU | 1872 | OD2 | ASP | F | 170 | 4486 | 4840 | 4628 | 99 | 4 | 176 | O |
| ATOM | 1873 | C | ASP | F | 170 | 0.722 | 59.831 | 37.167 | 1.00 | 19.47 | | C |
| ANISOU | 1873 | C | ASP | F | 170 | 2331 | 2555 | 2511 | −4 | 63 | 70 | C |
| ATOM | 1874 | O | ASP | F | 170 | 1.079 | 58.688 | 36.901 | 1.00 | 21.30 | | O |
| ANISOU | 1874 | O | ASP | F | 170 | 2575 | 2805 | 2711 | −100 | 227 | −201 | O |
| ATOM | 1876 | N | GLY | F | 171 | −0.556 | 60.184 | 37.251 | 1.00 | 17.25 | | N |
| ANISOU | 1876 | N | GLY | F | 171 | 2153 | 2160 | 2241 | −113 | 139 | −163 | N |
| ATOM | 1877 | CA | GLY | F | 171 | −1.620 | 59.220 | 36.995 | 1.00 | 15.70 | | C |
| ANISOU | 1877 | CA | GLY | F | 171 | 1985 | 2096 | 1884 | 33 | 26 | −160 | C |
| ATOM | 1880 | C | GLY | F | 171 | −1.803 | 58.999 | 35.510 | 1.00 | 14.75 | | C |
| ANISOU | 1880 | C | GLY | F | 171 | 1894 | 1808 | 1902 | −53 | 123 | −6 | C |
| ATOM | 1881 | O | GLY | F | 171 | −1.873 | 59.962 | 34.732 | 1.00 | 15.81 | | O |
| ANISOU | 1881 | O | GLY | F | 171 | 2129 | 1977 | 1900 | 79 | −31 | −53 | O |
| ATOM | 1883 | N | GLU | F | 172 | −1.886 | 57.736 | 35.104 | 1.00 | 14.97 | | N |
| ANISOU | 1883 | N | GLU | F | 172 | 1853 | 1982 | 1851 | −74 | 155 | −53 | N |
| ATOM | 1884 | CA | GLU | F | 172 | −2.197 | 57.450 | 33.718 | 1.00 | 15.61 | | C |
| ANISOU | 1884 | CA | GLU | F | 172 | 1789 | 2071 | 2068 | 20 | 41 | −77 | C |
| ATOM | 1886 | CB | GLU | F | 172 | −0.950 | 57.477 | 32.825 | 1.00 | 21.74 | | C |
| ANISOU | 1886 | CB | GLU | F | 172 | 2547 | 2967 | 2744 | −137 | 192 | −67 | C |
| ATOM | 1889 | CG | GLU | F | 172 | 0.024 | 56.380 | 33.056 | 1.00 | 22.88 | | C |
| ANISOU | 1889 | CG | GLU | F | 172 | 2749 | 2914 | 3029 | −53 | 234 | −86 | C |
| ATOM | 1892 | CD | GLU | F | 172 | 1.198 | 56.398 | 32.071 | 1.00 | 23.85 | | C |
| ANISOU | 1892 | CD | GLU | F | 172 | 2752 | 3107 | 3203 | −35 | 364 | 110 | C |
| ATOM | 1893 | OE1 | GLU | F | 172 | 1.357 | 57.372 | 31.298 | 1.00 | 24.37 | | O |
| ANISOU | 1893 | OE1 | GLU | F | 172 | 2645 | 3619 | 2994 | 480 | 232 | 340 | O |
| ATOM | 1894 | OE2 | GLU | F | 172 | 1.966 | 55.422 | 32.089 | 1.00 | 27.42 | | O |
| ANISOU | 1894 | OE2 | GLU | F | 172 | 3083 | 3424 | 3910 | 188 | 417 | 15 | O |
| ATOM | 1895 | C | GLU | F | 172 | −2.968 | 56.159 | 33.546 | 1.00 | 14.33 | | C |
| ANISOU | 1895 | C | GLU | F | 172 | 1878 | 1719 | 1848 | 114 | 116 | −41 | C |
| ATOM | 1896 | O | GLU | F | 172 | −2.824 | 55.212 | 34.322 | 1.00 | 15.78 | | O |
| ANISOU | 1896 | O | GLU | F | 172 | 2100 | 1903 | 1990 | 139 | −77 | 12 | O |
| ATOM | 1898 | N | LEU | F | 173 | −3.817 | 56.162 | 32.527 | 1.00 | 15.26 | | N |
| ANISOU | 1898 | N | LEU | F | 173 | 2026 | 1804 | 1967 | 102 | −97 | −46 | N |
| ATOM | 1899 | CA | LEU | F | 173 | −4.612 | 55.022 | 32.134 | 1.00 | 15.42 | | C |
| ANISOU | 1899 | CA | LEU | F | 173 | 1840 | 2063 | 1956 | 28 | −15 | −225 | C |
| ATOM | 1901 | CB | LEU | F | 173 | −6.100 | 55.378 | 32.180 | 1.00 | 16.05 | | C |
| ANISOU | 1901 | CB | LEU | F | 173 | 1961 | 2092 | 2044 | 30 | 90 | −148 | C |
| ATOM | 1904 | CG | LEU | F | 173 | −7.060 | 54.258 | 31.756 | 1.00 | 17.75 | | C |
| ANISOU | 1904 | CG | LEU | F | 173 | 2157 | 2431 | 2157 | 79 | −162 | −413 | C |
| ATOM | 1906 | CD1 | LEU | F | 173 | −7.108 | 53.184 | 32.806 | 1.00 | 18.22 | | C |
| ANISOU | 1906 | CD1 | LEU | F | 173 | 1996 | 2105 | 2820 | 83 | 36 | −327 | C |
| ATOM | 1910 | CD2 | LEU | F | 173 | −8.455 | 54.822 | 31.504 | 1.00 | 20.29 | | C |
| ANISOU | 1910 | CD2 | LEU | F | 173 | 2358 | 2773 | 2576 | 144 | −218 | −271 | C |
| ATOM | 1914 | C | LEU | F | 173 | −4.202 | 54.680 | 30.711 | 1.00 | 17.24 | | C |
| ANISOU | 1914 | C | LEU | F | 173 | 2379 | 2024 | 2147 | 6 | 96 | −128 | C |
| ATOM | 1915 | O | LEU | F | 173 | −4.208 | 55.552 | 29.839 | 1.00 | 17.45 | | O |
| ANISOU | 1915 | O | LEU | F | 173 | 2686 | 2094 | 1847 | 213 | 28 | −94 | O |
| ATOM | 1917 | N | ILE | F | 174 | −3.840 | 53.422 | 30.486 | 1.00 | 14.95 | | N |
| ANISOU | 1917 | N | ILE | F | 174 | 1940 | 1807 | 1931 | 87 | 23 | −78 | N |
| ATOM | 1918 | CA | ILE | F | 174 | −3.501 | 52.932 | 29.159 | 1.00 | 15.00 | | C |
| ANISOU | 1918 | CA | ILE | F | 174 | 1848 | 1886 | 1963 | 134 | −3 | 118 | C |
| ATOM | 1920 | CB | ILE | F | 174 | −2.067 | 52.389 | 29.092 | 1.00 | 15.25 | | C |
| ANISOU | 1920 | CB | ILE | F | 174 | 1913 | 2007 | 1874 | 274 | 20 | −61 | C |
| ATOM | 1922 | CG1 | ILE | F | 174 | −1.081 | 53.517 | 29.431 | 1.00 | 17.85 | | C |
| ANISOU | 1922 | CG1 | ILE | F | 174 | 1905 | 2359 | 2516 | 110 | −217 | 213 | C |
| ATOM | 1925 | CD1 | ILE | F | 174 | 0.398 | 53.098 | 29.524 | 1.00 | 18.77 | | C |
| ANISOU | 1925 | CD1 | ILE | F | 174 | 1970 | 2517 | 2644 | 171 | −13 | 85 | C |
| ATOM | 1929 | CG2 | ILE | F | 174 | −1.788 | 51.842 | 27.704 | 1.00 | 18.57 | | C |
| ANISOU | 1929 | CG2 | ILE | F | 174 | 2222 | 2626 | 2205 | 324 | −33 | −210 | C |
| ATOM | 1933 | C | ILE | F | 174 | −4.486 | 51.847 | 28.785 | 1.00 | 14.91 | | C |
| ANISOU | 1933 | C | ILE | F | 174 | 2001 | 1818 | 1844 | 8 | −102 | −40 | C |
| ATOM | 1934 | O | ILE | F | 174 | −4.676 | 50.895 | 29.530 | 1.00 | 16.31 | | O |
| ANISOU | 1934 | O | ILE | F | 174 | 2305 | 2054 | 1836 | −202 | −52 | −41 | O |
| ATOM | 1936 | N | VAL | F | 175 | −5.135 | 52.029 | 27.638 | 1.00 | 14.84 | | N |
| ANISOU | 1936 | N | VAL | F | 175 | 2047 | 1783 | 1807 | −121 | −149 | 61 | N |
| ATOM | 1937 | CA | VAL | F | 175 | −6.032 | 51.041 | 27.067 | 1.00 | 15.25 | | C |
| ANISOU | 1937 | CA | VAL | F | 175 | 1981 | 1876 | 1934 | −193 | 82 | −26 | C |
| ATOM | 1939 | CB | VAL | F | 175 | −7.446 | 51.607 | 26.895 | 1.00 | 15.72 | | C |
| ANISOU | 1939 | CB | VAL | F | 175 | 2027 | 1993 | 1952 | −73 | 37 | 16 | C |
| ATOM | 1941 | CG1 | VAL | F | 175 | −8.373 | 50.549 | 26.339 | 1.00 | 17.65 | | C |
| ANISOU | 1941 | CG1 | VAL | F | 175 | 2272 | 2195 | 2236 | −116 | −74 | −24 | C |
| ATOM | 1945 | CG2 | VAL | F | 175 | −7.975 | 52.128 | 28.229 | 1.00 | 16.50 | | C |
| ANISOU | 1945 | CG2 | VAL | F | 175 | 2157 | 2099 | 2013 | −49 | 103 | −100 | C |
| ATOM | 1949 | C | VAL | F | 175 | −5.455 | 50.660 | 25.708 | 1.00 | 14.19 | | C |
| ANISOU | 1949 | C | VAL | F | 175 | 1742 | 1777 | 1873 | −80 | −8 | −147 | C |
| ATOM | 1950 | O | VAL | F | 175 | −5.279 | 51.524 | 24.854 | 1.00 | 15.82 | | O |
| ANISOU | 1950 | O | VAL | F | 175 | 2386 | 1775 | 1847 | −60 | 55 | 63 | O |
| ATOM | 1952 | N | VAL | F | 176 | −5.117 | 49.389 | 25.519 | 1.00 | 14.60 | | N |
| ANISOU | 1952 | N | VAL | F | 176 | 1944 | 1777 | 1824 | −104 | 9 | −57 | N |
| ATOM | 1953 | CA | VAL | F | 176 | −4.452 | 48.966 | 24.289 | 1.00 | 14.55 | | C |
| ANISOU | 1953 | CA | VAL | F | 176 | 1886 | 1793 | 1846 | −115 | 63 | 27 | C |
| ATOM | 1955 | CB | VAL | F | 176 | −2.912 | 48.993 | 24.431 | 1.00 | 16.92 | | C |
| ANISOU | 1955 | CB | VAL | F | 176 | 2133 | 2255 | 2038 | −54 | −35 | 61 | C |
| ATOM | 1957 | CG1 | VAL | F | 176 | −2.447 | 48.219 | 25.603 | 1.00 | 19.92 | | C |
| ANISOU | 1957 | CG1 | VAL | F | 176 | 2358 | 2567 | 2642 | −121 | 162 | 41 | C |
| ATOM | 1961 | CG2 | VAL | F | 176 | −2.224 | 48.549 | 23.150 | 1.00 | 16.21 | | C |
| ANISOU | 1961 | CG2 | VAL | F | 176 | 1751 | 2303 | 2102 | 41 | −60 | −147 | C |
| ATOM | 1965 | C | VAL | F | 176 | −4.974 | 47.628 | 23.781 | 1.00 | 13.32 | | C |
| ANISOU | 1965 | C | VAL | F | 176 | 1559 | 1698 | 1801 | 144 | 206 | −80 | C |
| ATOM | 1966 | O | VAL | F | 176 | −5.036 | 46.648 | 24.513 | 1.00 | 14.24 | | O |
| ANISOU | 1966 | O | VAL | F | 176 | 1786 | 1820 | 1802 | 37 | −31 | 104 | O |
| ATOM | 1968 | N | GLN | F | 177 | −5.335 | 47.609 | 22.502 | 1.00 | 13.81 | | N |
| ANISOU | 1968 | N | GLN | F | 177 | 1738 | 1686 | 1820 | −24 | 86 | 69 | N |
| ATOM | 1969 | CA | GLN | F | 177 | −5.921 | 46.444 | 21.862 | 1.00 | 13.58 | | C |
| ANISOU | 1969 | CA | GLN | F | 177 | 1720 | 1710 | 1730 | 25 | 38 | −17 | C |
| ATOM | 1971 | CB | GLN | F | 177 | −6.580 | 46.891 | 20.556 | 1.00 | 14.37 | | C |
| ANISOU | 1971 | CB | GLN | F | 177 | 1696 | 1875 | 1887 | −57 | −16 | −115 | C |
| ATOM | 1974 | CG | GLN | F | 177 | −7.376 | 45.845 | 19.828 | 1.00 | 14.39 | | C |
| ANISOU | 1974 | CG | GLN | F | 177 | 1873 | 1726 | 1868 | −135 | −6 | 22 | C |
| ATOM | 1977 | CD | GLN | F | 177 | −7.927 | 46.370 | 18.527 | 1.00 | 14.67 | | C |
| ANISOU | 1977 | CD | GLN | F | 177 | 1847 | 1902 | 1824 | −139 | −88 | −26 | C |
| ATOM | 1978 | OE1 | GLN | F | 177 | −7.303 | 47.228 | 17.875 | 1.00 | 15.37 | | O |
| ANISOU | 1978 | OE1 | GLN | F | 177 | 1984 | 1919 | 1936 | −331 | −8 | 1 | O |
| ATOM | 1979 | NE2 | GLN | F | 177 | −9.089 | 45.848 | 18.118 | 1.00 | 15.69 | | N |
| ANISOU | 1979 | NE2 | GLN | F | 177 | 1737 | 1936 | 2288 | −228 | −15 | −30 | N |
| ATOM | 1982 | C | GLN | F | 177 | −4.851 | 45.393 | 21.581 | 1.00 | 14.62 | | C |
| ANISOU | 1982 | C | GLN | F | 177 | 1773 | 1788 | 1994 | 67 | 43 | 76 | C |
| ATOM | 1983 | O | GLN | F | 177 | −3.795 | 45.718 | 21.034 | 1.00 | 16.34 | | O |
| ANISOU | 1983 | O | GLN | F | 177 | 1974 | 1827 | 2407 | −26 | 359 | 160 | O |
| ATOM | 1985 | N | LEU | F | 178 | −5.141 | 44.144 | 21.943 | 1.00 | 13.42 | | N |
| ANISOU | 1985 | N | LEU | F | 178 | 1639 | 1641 | 1818 | 8 | 185 | −54 | N |
| ATOM | 1986 | CA | LEU | F | 178 | −4.253 | 43.006 | 21.691 | 1.00 | 14.36 | | C |
| ANISOU | 1986 | CA | LEU | F | 178 | 1740 | 1740 | 1974 | 35 | 80 | −42 | C |
| ATOM | 1988 | CB | LEU | F | 178 | −4.153 | 42.127 | 22.931 | 1.00 | 14.43 | | C |
| ANISOU | 1988 | CB | LEU | F | 178 | 1695 | 1784 | 2003 | −78 | −68 | 77 | C |
| ATOM | 1991 | CG | LEU | F | 178 | −3.707 | 42.819 | 24.208 | 1.00 | 14.48 | | C |
| ANISOU | 1991 | CG | LEU | F | 178 | 1898 | 1881 | 1721 | 17 | 89 | 62 | C |
| ATOM | 1993 | CD1 | LEU | F | 178 | −3.728 | 41.823 | 25.330 | 1.00 | 15.93 | | C |
| ANISOU | 1993 | CD1 | LEU | F | 178 | 2077 | 2299 | 1675 | −125 | −105 | 56 | C |
| ATOM | 1997 | CD2 | LEU | F | 178 | −2.330 | 43.430 | 24.054 | 1.00 | 16.96 | | C |
| ANISOU | 1997 | CD2 | LEU | F | 178 | 1926 | 2313 | 2203 | 68 | −64 | 52 | C |
| ATOM | 2001 | C | LEU | F | 178 | −4.693 | 42.117 | 20.541 | 1.00 | 14.89 | | C |
| ANISOU | 2001 | C | LEU | F | 178 | 1882 | 1903 | 1870 | 27 | −147 | 73 | C |
| ATOM | 2002 | O | LEU | F | 178 | −3.865 | 41.431 | 19.948 | 1.00 | 15.70 | | O |
| ANISOU | 2002 | O | LEU | F | 178 | 1852 | 2134 | 1979 | 160 | −42 | −27 | O |
| ATOM | 2004 | N | THR | F | 179 | −5.988 | 42.085 | 20.260 | 1.00 | 13.89 | | N |
| ANISOU | 2004 | N | THR | F | 179 | 1688 | 1680 | 1908 | 148 | −21 | −131 | N |
| ATOM | 2005 | CA | THR | F | 179 | −6.506 | 41.276 | 19.171 | 1.00 | 14.91 | | C |
| ANISOU | 2005 | CA | THR | F | 179 | 1766 | 1880 | 2019 | −38 | −73 | −50 | C |
| ATOM | 2007 | CB | THR | F | 179 | −7.210 | 39.995 | 19.662 | 1.00 | 15.23 | | C |
| ANISOU | 2007 | CB | THR | F | 179 | 1985 | 1906 | 1895 | −35 | 100 | −103 | C |
| ATOM | 2009 | OG1 | THR | F | 179 | −8.342 | 40.340 | 20.465 | 1.00 | 16.72 | | O |
| ANISOU | 2009 | OG1 | THR | F | 179 | 2092 | 2059 | 2201 | −136 | 436 | −110 | O |
| ATOM | 2011 | CG2 | THR | F | 179 | −6.265 | 39.158 | 20.473 | 1.00 | 16.64 | | C |
| ANISOU | 2011 | CG2 | THR | F | 179 | 2385 | 1915 | 2022 | 4 | 42 | −65 | C |
| ATOM | 2015 | C | THR | F | 179 | −7.477 | 42.106 | 18.354 | 1.00 | 15.55 | | C |
| ANISOU | 2015 | C | THR | F | 179 | 1987 | 1949 | 1971 | −162 | −114 | 3 | C |
| ATOM | 2016 | O | THR | F | 179 | −8.160 | 42.964 | 18.896 | 1.00 | 15.86 | | O |
| ANISOU | 2016 | O | THR | F | 179 | 1790 | 1938 | 2298 | −39 | −201 | −56 | O |
| ATOM | 2018 | N | PRO | F | 180 | −7.531 | 41.859 | 17.042 | 1.00 | 17.37 | | N |
| ANISOU | 2018 | N | PRO | F | 180 | 2350 | 2131 | 2117 | 188 | −162 | −16 | N |
| ATOM | 2019 | CA | PRO | F | 180 | −8.405 | 42.622 | 16.168 | 1.00 | 17.80 | | C |
| ANISOU | 2019 | CA | PRO | F | 180 | 2234 | 2294 | 2234 | 159 | −188 | −23 | C |
| ATOM | 2021 | CB | PRO | F | 180 | −7.852 | 42.306 | 14.780 | 1.00 | 20.17 | | C |
| ANISOU | 2021 | CB | PRO | F | 180 | 2376 | 2879 | 2406 | 151 | −192 | 76 | C |
| ATOM | 2024 | CG | PRO | F | 180 | −7.323 | 40.927 | 14.908 | 1.00 | 20.77 | | C |
| ANISOU | 2024 | CG | PRO | F | 180 | 2704 | 2823 | 2362 | 57 | −15 | −138 | C |
| ATOM | 2027 | CD | PRO | F | 180 | −6.740 | 40.864 | 16.295 | 1.00 | 18.87 | | C |
| ANISOU | 2027 | CD | PRO | F | 180 | 2430 | 2596 | 2141 | 182 | 205 | −156 | C |
| ATOM | 2030 | C | PRO | F | 180 | −9.865 | 42.193 | 16.293 | 1.00 | 18.44 | | C |
| ANISOU | 2030 | C | PRO | F | 180 | 2364 | 2337 | 2304 | 129 | −120 | 56 | C |
| ATOM | 2031 | O | PRO | F | 180 | −10.174 | 41.097 | 16.751 | 1.00 | 21.99 | | O |
| ANISOU | 2031 | O | PRO | F | 180 | 2676 | 2735 | 2942 | 287 | −278 | 186 | O |
| ATOM | 2032 | N | GLY | F | 181 | −10.760 | 43.074 | 15.882 | 1.00 | 19.14 | | N |
| ANISOU | 2032 | N | GLY | F | 181 | 2262 | 2461 | 2547 | 189 | 52 | 63 | N |
| ATOM | 2033 | CA | GLY | F | 181 | −12.176 | 42.764 | 15.893 | 1.00 | 17.72 | | C |
| ANISOU | 2033 | CA | GLY | F | 181 | 2283 | 2303 | 2144 | 1 | 92 | −26 | C |
| ATOM | 2036 | C | GLY | F | 181 | −12.944 | 43.744 | 15.046 | 1.00 | 18.20 | | C |
| ANISOU | 2036 | C | GLY | F | 181 | 2174 | 2281 | 2458 | −16 | 99 | 45 | C |
| ATOM | 2037 | O | GLY | F | 181 | −12.377 | 44.659 | 14.452 | 1.00 | 19.48 | | O |
| ANISOU | 2037 | O | GLY | F | 181 | 2159 | 2484 | 2757 | −102 | 21 | 320 | O |
| ATOM | 2039 | N | TYR | F | 182 | −14.247 | 43.542 | 14.994 | 1.00 | 16.77 | | N |
| ANISOU | 2039 | N | TYR | F | 182 | 2066 | 1897 | 2407 | −237 | −17 | −63 | N |
| ATOM | 2040 | CA | TYR | F | 182 | −15.113 | 44.415 | 14.250 | 1.00 | 18.04 | | C |
| ANISOU | 2040 | CA | TYR | F | 182 | 2290 | 2171 | 2390 | −75 | −102 | −123 | C |
| ATOM | 2042 | CB | TYR | F | 182 | −16.559 | 43.957 | 14.407 | 1.00 | 21.95 | | C |
| ANISOU | 2042 | CB | TYR | F | 182 | 2637 | 2756 | 2943 | −170 | 2 | 44 | C |
| ATOM | 2045 | CG | TYR | F | 182 | −17.539 | 44.951 | 13.873 | 1.00 | 22.91 | | C |
| ANISOU | 2045 | CG | TYR | F | 182 | 2773 | 2754 | 3177 | −159 | 90 | 84 | C |
| ATOM | 2046 | CD1 | TYR | F | 182 | −17.898 | 44.940 | 12.534 | 1.00 | 23.56 | | C |
| ANISOU | 2046 | CD1 | TYR | F | 182 | 2741 | 2979 | 3229 | −204 | −10 | 203 | C |
| ATOM | 2048 | CE1 | TYR | F | 182 | −18.793 | 45.863 | 12.028 | 1.00 | 23.27 | | C |
| ANISOU | 2048 | CE1 | TYR | F | 182 | 2824 | 2842 | 3173 | −56 | 51 | 162 | C |
| ATOM | 2050 | CZ | TYR | F | 182 | −19.333 | 46.811 | 12.864 | 1.00 | 22.67 | | C |
| ANISOU | 2050 | CZ | TYR | F | 182 | 2774 | 2857 | 2981 | −168 | 7 | 88 | C |
| ATOM | 2051 | OH | TYR | F | 182 | −20.223 | 47.720 | 12.350 | 1.00 | 24.51 | | O |
| ANISOU | 2051 | OH | TYR | F | 182 | 2952 | 3056 | 3302 | −15 | 188 | 237 | O |
| ATOM | 2053 | CE2 | TYR | F | 182 | −18.983 | 46.850 | 14.205 | 1.00 | 24.49 | | C |
| ANISOU | 2053 | CE2 | TYR | F | 182 | 3017 | 3108 | 3179 | 99 | 89 | 177 | C |
| ATOM | 2055 | CD2 | TYR | F | 182 | −18.089 | 45.922 | 14.700 | 1.00 | 22.68 | | C |
| ANISOU | 2055 | CD2 | TYR | F | 182 | 2677 | 3027 | 2911 | 30 | 28 | 147 | C |
| ATOM | 2057 | C | TYR | F | 182 | −14.969 | 45.849 | 14.751 | 1.00 | 17.50 | | C |
| ANISOU | 2057 | C | TYR | F | 182 | 2436 | 2036 | 2178 | −63 | −147 | −38 | C |
| ATOM | 2058 | O | TYR | F | 182 | −14.967 | 46.075 | 15.957 | 1.00 | 16.98 | | O |
| ANISOU | 2058 | O | TYR | F | 182 | 2363 | 2155 | 1930 | −215 | −220 | 197 | O |
| ATOM | 2060 | N | CYS | F | 183 | −14.855 | 46.802 | 13.825 | 1.00 | 17.83 | | N |
| ANISOU | 2060 | N | CYS | F | 183 | 2392 | 2281 | 2101 | −272 | −120 | −113 | N |
| ATOM | 2061 | CA | CYS | F | 183 | −14.863 | 48.226 | 14.159 | 1.00 | 20.04 | | C |
| ANISOU | 2061 | CA | CYS | F | 183 | 2682 | 2467 | 2465 | −89 | −25 | −173 | C |
| ATOM | 2063 | CB | CYS | F | 183 | −13.664 | 48.942 | 13.551 | 1.00 | 21.00 | | C |
| ANISOU | 2063 | CB | CYS | F | 183 | 2866 | 2599 | 2514 | −233 | 45 | −69 | C |
| ATOM | 2066 | SG | CYS | F | 183 | −12.087 | 48.424 | 14.206 | 1.00 | 24.51 | | S |
| ANISOU | 2066 | SG | CYS | F | 183 | 3195 | 3064 | 3053 | −242 | 88 | 108 | S |
| ATOM | 2068 | C | CYS | F | 183 | −16.117 | 48.875 | 13.626 | 1.00 | 20.52 | | C |
| ANISOU | 2068 | C | CYS | F | 183 | 2751 | 2357 | 2690 | −60 | 4 | 58 | C |
| ATOM | 2069 | O | CYS | F | 183 | −16.496 | 48.636 | 12.481 | 1.00 | 21.28 | | O |
| ANISOU | 2069 | O | CYS | F | 183 | 3001 | 2574 | 2509 | −197 | 56 | 224 | O |
| ATOM | 2071 | N | ALA | F | 184 | −16.746 | 49.707 | 14.452 | 1.00 | 21.80 | | N |
| ANISOU | 2071 | N | ALA | F | 184 | 2904 | 2794 | 2584 | 0 | −97 | −97 | N |
| ATOM | 2072 | CA | ALA | F | 184 | −17.899 | 50.493 | 14.027 | 1.00 | 25.91 | | C |
| ANISOU | 2072 | CA | ALA | F | 184 | 3239 | 3284 | 3319 | 110 | 27 | −52 | C |
| ATOM | 2074 | CB | ALA | F | 184 | −18.520 | 51.209 | 15.221 | 1.00 | 27.04 | | C |
| ANISOU | 2074 | CB | ALA | F | 184 | 3458 | 3397 | 3415 | 148 | 86 | 42 | C |
| ATOM | 2078 | C | ALA | F | 184 | −17.472 | 51.512 | 12.970 | 1.00 | 28.78 | | C |
| ANISOU | 2078 | C | ALA | F | 184 | 3607 | 3650 | 3679 | 9 | 81 | 68 | C |
| ATOM | 2079 | O | ALA | F | 184 | −16.365 | 52.051 | 13.045 | 1.00 | 30.99 | | O |
| ANISOU | 2079 | O | ALA | F | 184 | 3798 | 3987 | 3989 | −121 | −51 | 119 | O |
| ATOM | 2081 | N | PRO | F | 185 | −18.339 | 51.774 | 11.977 | 1.00 | 29.74 | | N |
| ANISOU | 2081 | N | PRO | F | 185 | 3797 | 3733 | 3770 | 16 | 68 | 76 | N |
| ATOM | 2082 | CA | PRO | F | 185 | −18.091 | 52.821 | 10.978 | 1.00 | 30.44 | | C |
| ANISOU | 2082 | CA | PRO | F | 185 | 3945 | 3819 | 3799 | 16 | 88 | 86 | C |
| ATOM | 2084 | CB | PRO | F | 185 | −19.431 | 52.925 | 10.246 | 1.00 | 30.69 | | C |
| ANISOU | 2084 | CB | PRO | F | 185 | 3958 | 3835 | 3865 | −17 | 16 | 30 | C |
| ATOM | 2087 | CG | PRO | F | 185 | −20.040 | 51.589 | 10.385 | 1.00 | 29.98 | | C |
| ANISOU | 2087 | CG | PRO | F | 185 | 3813 | 3743 | 3832 | −4 | 14 | 49 | C |
| ATOM | 2090 | CD | PRO | F | 185 | −19.604 | 51.061 | 11.714 | 1.00 | 29.33 | | C |
| ANISOU | 2090 | CD | PRO | F | 185 | 3753 | 3640 | 3750 | −31 | −3 | 7 | C |
| ATOM | 2093 | C | PRO | F | 185 | −17.717 | 54.178 | 11.580 | 1.00 | 33.44 | | C |
| ANISOU | 2093 | C | PRO | F | 185 | 4416 | 4061 | 4226 | −47 | 54 | −29 | C |
| ATOM | 2094 | O | PRO | F | 185 | −18.101 | 54.484 | 12.710 | 1.00 | 36.58 | | O |
| ANISOU | 2094 | O | PRO | F | 185 | 4864 | 4486 | 4548 | −7 | 186 | −16 | O |
| ATOM | 2095 | C1 | NAG | F | 200 | −21.028 | 63.219 | 31.740 | 1.00 | 30.10 | | C |
| ANISOU | 2095 | C1 | NAG | F | 200 | 3763 | 3900 | 3773 | 79 | 77 | −39 | C |
| ATOM | 2098 | C2 | NAG | F | 200 | −22.434 | 63.530 | 31.236 | 1.00 | 32.17 | | C |
| ANISOU | 2098 | C2 | NAG | F | 200 | 4039 | 4137 | 4045 | 48 | −28 | 35 | C |
| ATOM | 2100 | N2 | NAG | F | 200 | −22.442 | 64.540 | 30.186 | 1.00 | 33.66 | | N |
| ANISOU | 2100 | N2 | NAG | F | 200 | 4279 | 4333 | 4176 | 20 | 2 | 47 | N |
| ATOM | 2102 | C7 | NAG | F | 200 | −22.582 | 64.219 | 28.898 | 1.00 | 34.85 | | C |
| ANISOU | 2102 | C7 | NAG | F | 200 | 4410 | 4563 | 4266 | −52 | 9 | −9 | C |
| ATOM | 2103 | O7 | NAG | F | 200 | −21.706 | 64.433 | 28.060 | 1.00 | 38.15 | | O |
| ANISOU | 2103 | O7 | NAG | F | 200 | 4794 | 5087 | 4611 | −225 | 81 | 70 | O |
| ATOM | 2104 | C8 | NAG | F | 200 | −23.867 | 63.563 | 28.486 | 1.00 | 33.97 | | C |
| ANISOU | 2104 | C8 | NAG | F | 200 | 4326 | 4405 | 4175 | −56 | 9 | −5 | C |
| ATOM | 2108 | C3 | NAG | F | 200 | −23.261 | 63.969 | 32.432 | 1.00 | 32.46 | | C |
| ANISOU | 2108 | C3 | NAG | F | 200 | 4028 | 4205 | 4099 | 63 | 32 | 56 | C |
| ATOM | 2110 | O3 | NAG | F | 200 | −24.561 | 64.337 | 32.019 | 1.00 | 34.73 | | O |
| ANISOU | 2110 | O3 | NAG | F | 200 | 4185 | 4540 | 4470 | 129 | −64 | 148 | O |
| ATOM | 2112 | C4 | NAG | F | 200 | −23.349 | 62.826 | 33.438 | 1.00 | 32.07 | | C |
| ANISOU | 2112 | C4 | NAG | F | 200 | 3989 | 4144 | 4052 | 107 | 7 | 31 | C |
| ATOM | 2114 | O4 | NAG | F | 200 | −23.864 | 63.340 | 34.653 | 1.00 | 32.07 | | O |
| ANISOU | 2114 | O4 | NAG | F | 200 | 4103 | 4211 | 3868 | 68 | −44 | 95 | O |
| ATOM | 2116 | C5 | NAG | F | 200 | −22.001 | 62.134 | 33.704 | 1.00 | 32.25 | | C |
| ANISOU | 2116 | C5 | NAG | F | 200 | 3995 | 4187 | 4069 | 81 | 53 | 83 | C |
| ATOM | 2118 | C6 | NAG | F | 200 | −22.218 | 60.673 | 34.102 | 1.00 | 32.38 | | C |
| ANISOU | 2118 | C6 | NAG | F | 200 | 4000 | 4188 | 4115 | 23 | 8 | 53 | C |
| ATOM | 2121 | O6 | NAG | F | 200 | −22.509 | 60.569 | 35.475 | 1.00 | 35.19 | | O |
| ANISOU | 2121 | O6 | NAG | F | 200 | 4485 | 4698 | 4189 | 15 | −17 | 150 | O |
| ATOM | 2123 | O5 | NAG | F | 200 | −21.100 | 62.103 | 32.604 | 1.00 | 31.54 | | O |
| ANISOU | 2123 | O5 | NAG | F | 200 | 3923 | 4087 | 3971 | 40 | 94 | 65 | O |
| ATOM | 2124 | S | SO4 | F | 201 | 4.476 | 61.749 | 28.616 | 1.00 | 17.31 | | S |
| ANISOU | 2124 | S | SO4 | F | 201 | 2363 | 2131 | 2082 | 336 | −232 | 432 | S |
| ATOM | 2125 | O1 | SO4 | F | 201 | 4.876 | 60.864 | 27.532 | 1.00 | 18.13 | | O |
| ANISOU | 2125 | O1 | SO4 | F | 201 | 2426 | 2211 | 2250 | 545 | −306 | 301 | O |
| ATOM | 2126 | O2 | SO4 | F | 201 | 3.472 | 62.689 | 28.124 | 1.00 | 18.62 | | O |
| ANISOU | 2126 | O2 | SO4 | F | 201 | 2138 | 2646 | 2289 | 671 | −211 | 354 | O |
| ATOM | 2127 | O3 | SO4 | F | 201 | 5.624 | 62.472 | 29.139 | 1.00 | 20.60 | | O |
| ANISOU | 2127 | O3 | SO4 | F | 201 | 2604 | 2640 | 2581 | 66 | −289 | 280 | O |
| ATOM | 2128 | O4 | SO4 | F | 201 | 3.892 | 60.946 | 29.692 | 1.00 | 20.33 | | O |
| ANISOU | 2128 | O4 | SO4 | F | 201 | 3006 | 2470 | 2246 | 80 | −226 | 479 | O |
| ATOM | 2129 | O | HOH | W | 102 | −1.272 | 45.036 | 20.445 | 1.00 | 19.51 | | O |
| ANISOU | 2129 | O | HOH | W | 102 | 2034 | 1797 | 3581 | 92 | 160 | 229 | O |
| ATOM | 2132 | O | HOH | W | 103 | −20.926 | 47.481 | 24.342 | 1.00 | 22.65 | | O |
| ANISOU | 2132 | O | HOH | W | 103 | 2402 | 2974 | 3228 | −266 | −773 | −607 | O |
| ATOM | 2135 | O | HOH | W | 104 | −13.426 | 58.236 | 34.448 | 1.00 | 16.92 | | O |
| ANISOU | 2135 | O | HOH | W | 104 | 2421 | 1730 | 2276 | −314 | −84 | −644 | O |
| ATOM | 2138 | O | HOH | W | 105 | −1.818 | 62.903 | 22.227 | 1.00 | 21.85 | | O |
| ANISOU | 2138 | O | HOH | W | 105 | 2615 | 2679 | 3008 | 247 | −47 | −14 | O |
| ATOM | 2141 | O | HOH | W | 107 | −12.718 | 55.843 | 20.134 | 1.00 | 22.61 | | O |
| ANISOU | 2141 | O | HOH | W | 107 | 2911 | 2872 | 2805 | −112 | −567 | −91 | O |
| ATOM | 2144 | O | HOH | W | 108 | −11.623 | 50.947 | 19.906 | 1.00 | 16.14 | | O |
| ANISOU | 2144 | O | HOH | W | 108 | 2124 | 1789 | 2217 | −38 | −476 | −50 | O |
| ATOM | 2147 | O | HOH | W | 109 | −1.948 | 48.448 | 42.204 | 1.00 | 19.71 | | O |
| ANISOU | 2147 | O | HOH | W | 109 | 2800 | 2090 | 2595 | −30 | −29 | 277 | O |
| ATOM | 2150 | O | HOH | W | 110 | −17.361 | 47.014 | 34.149 | 1.00 | 25.48 | | O |
| ANISOU | 2150 | O | HOH | W | 110 | 3026 | 3201 | 3451 | −999 | −238 | −489 | O |
| ATOM | 2153 | O | HOH | W | 111 | −9.232 | 65.632 | 42.922 | 1.00 | 18.09 | | O |
| ANISOU | 2153 | O | HOH | W | 111 | 2511 | 2094 | 2266 | −142 | 261 | −755 | O |
| ATOM | 2156 | O | HOH | W | 112 | −10.785 | 40.613 | 33.197 | 1.00 | 21.81 | | O |
| ANISOU | 2156 | O | HOH | W | 112 | 2135 | 3346 | 2805 | −89 | 343 | 57 | O |
| ATOM | 2159 | O | HOH | W | 113 | −10.525 | 39.753 | 19.175 | 1.00 | 23.82 | | O |
| ANISOU | 2159 | O | HOH | W | 113 | 2861 | 3205 | 2985 | −233 | −185 | −538 | O |
| ATOM | 2162 | O | HOH | W | 114 | 4.026 | 55.134 | 21.171 | 1.00 | 24.30 | | O |
| ANISOU | 2162 | O | HOH | W | 114 | 3574 | 2486 | 3171 | 352 | 107 | −182 | O |
| ATOM | 2165 | O | HOH | W | 115 | −11.479 | 54.725 | 17.961 | 1.00 | 25.20 | | O |
| ANISOU | 2165 | O | HOH | W | 115 | 3217 | 2943 | 3415 | 184 | −131 | −1 | O |
| ATOM | 2168 | O | HOH | W | 116 | −4.659 | 65.575 | 41.220 | 1.00 | 22.14 | | O |
| ANISOU | 2168 | O | HOH | W | 116 | 3548 | 2433 | 2432 | 52 | −340 | −517 | O |
| ATOM | 2171 | O | HOH | W | 117 | −13.802 | 64.113 | 28.720 | 1.00 | 31.81 | | O |
| ANISOU | 2171 | O | HOH | W | 117 | 4288 | 3636 | 4162 | 159 | −319 | 130 | O |
| ATOM | 2174 | O | HOH | W | 118 | −1.998 | 62.708 | 37.586 | 1.00 | 17.07 | | O |
| ANISOU | 2174 | O | HOH | W | 118 | 2553 | 1889 | 2041 | −188 | −179 | −308 | O |
| ATOM | 2177 | O | HOH | W | 119 | 2.433 | 54.015 | 40.803 | 1.00 | 33.43 | | O |
| ANISOU | 2177 | O | HOH | W | 119 | 4060 | 4162 | 4478 | 91 | 21 | −241 | O |
| ATOM | 2180 | O | HOH | W | 120 | −7.604 | 47.458 | 15.112 | 1.00 | 29.94 | | O |
| ANISOU | 2180 | O | HOH | W | 120 | 4288 | 4167 | 2918 | −721 | 244 | −132 | O |
| ATOM | 2183 | O | HOH | W | 121 | −8.607 | 66.352 | 33.122 | 1.00 | 31.41 | | O |
| ANISOU | 2183 | O | HOH | W | 121 | 3921 | 4032 | 3979 | 66 | −216 | 69 | O |
| ATOM | 2186 | O | HOH | W | 122 | −19.093 | 62.559 | 36.968 | 1.00 | 30.39 | | O |
| ANISOU | 2186 | O | HOH | W | 122 | 3265 | 4238 | 4042 | 459 | −119 | −644 | O |
| ATOM | 2189 | O | HOH | W | 123 | −18.077 | 53.250 | 22.952 | 1.00 | 32.72 | | O |
| ANISOU | 2189 | O | HOH | W | 123 | 3686 | 4336 | 4407 | −18 | −219 | −207 | O |
| ATOM | 2192 | O | HOH | W | 124 | 3.157 | 57.818 | 20.374 | 1.00 | 31.42 | | O |
| ANISOU | 2192 | O | HOH | W | 124 | 3487 | 4465 | 3985 | 385 | 129 | 62 | O |
| ATOM | 2195 | O | HOH | W | 125 | −5.989 | 48.127 | 48.178 | 1.00 | 36.72 | | O |
| ANISOU | 2195 | O | HOH | W | 125 | 5025 | 5169 | 3755 | −106 | 320 | 91 | O |
| ATOM | 2198 | O | HOH | W | 126 | −18.812 | 57.599 | 42.091 | 1.00 | 38.14 | | O |
| ANISOU | 2198 | O | HOH | W | 126 | 4296 | 4971 | 5223 | 183 | 479 | −380 | O |
| ATOM | 2201 | O | HOH | W | 127 | −9.787 | 45.954 | 15.229 | 1.00 | 21.65 | | O |
| ANISOU | 2201 | O | HOH | W | 127 | 3112 | 2894 | 2218 | −72 | −517 | −283 | O |
| ATOM | 2204 | O | HOH | W | 128 | 0.414 | 55.247 | 20.342 | 1.00 | 21.30 | | O |
| ANISOU | 2204 | O | HOH | W | 128 | 2549 | 2697 | 2848 | −88 | 50 | 54 | O |
| ATOM | 2207 | O | HOH | W | 129 | −7.963 | 60.952 | 22.200 | 1.00 | 25.36 | | O |
| ANISOU | 2207 | O | HOH | W | 129 | 3719 | 2190 | 3725 | 156 | −527 | 31 | O |
| ATOM | 2210 | O | HOH | W | 130 | −8.102 | 51.819 | 44.462 | 1.00 | 23.55 | | O |
| ANISOU | 2210 | O | HOH | W | 130 | 3185 | 2475 | 3285 | 39 | 3 | −438 | O |
| ATOM | 2213 | O | HOH | W | 131 | −1.453 | 50.223 | 14.260 | 1.00 | 29.43 | | O |
| ANISOU | 2213 | O | HOH | W | 131 | 3173 | 4383 | 3623 | −157 | 632 | 173 | O |
| ATOM | 2216 | O | HOH | W | 132 | −16.971 | 40.038 | 24.996 | 1.00 | 20.61 | | O |
| ANISOU | 2216 | O | HOH | W | 132 | 2298 | 2606 | 2926 | −216 | −222 | −378 | O |
| ATOM | 2219 | O | HOH | W | 133 | 7.688 | 54.238 | 42.249 | 1.00 | 31.68 | | O |
| ANISOU | 2219 | O | HOH | W | 133 | 4315 | 3951 | 3770 | 568 | 419 | −398 | O |
| ATOM | 2222 | O | HOH | W | 134 | −19.445 | 44.958 | 18.832 | 1.00 | 26.98 | | O |
| ANISOU | 2222 | O | HOH | W | 134 | 3270 | 3364 | 3616 | −241 | −109 | −222 | O |
| ATOM | 2225 | O | HOH | W | 135 | −6.264 | 67.507 | 42.525 | 1.00 | 30.67 | | O |
| ANISOU | 2225 | O | HOH | W | 135 | 3791 | 3729 | 4131 | −13 | −160 | −342 | O |
| ATOM | 2228 | O | HOH | W | 136 | 4.438 | 64.688 | 46.307 | 1.00 | 24.99 | | O |
| ANISOU | 2228 | O | HOH | W | 136 | 3081 | 2587 | 3828 | −243 | 275 | −181 | O |
| ATOM | 2231 | O | HOH | W | 137 | −0.433 | 52.710 | 13.943 | 1.00 | 35.50 | | O |
| ANISOU | 2231 | O | HOH | W | 137 | 4258 | 4565 | 4664 | 247 | 2 | 85 | O |
| ATOM | 2234 | O | HOH | W | 138 | −19.854 | 50.788 | 28.894 | 1.00 | 32.97 | | O |
| ANISOU | 2234 | O | HOH | W | 138 | 4468 | 3827 | 4231 | 123 | 627 | −368 | O |
| ATOM | 2237 | O | HOH | W | 139 | −1.455 | 44.037 | 38.205 | 1.00 | 31.94 | | O |
| ANISOU | 2237 | O | HOH | W | 139 | 4513 | 3796 | 3824 | 158 | −473 | 227 | O |
| ATOM | 2240 | O | HOH | W | 140 | −15.747 | 42.294 | 36.438 | 1.00 | 37.11 | | O |
| ANISOU | 2240 | O | HOH | W | 140 | 4789 | 4384 | 4927 | 52 | −60 | 0 | O |
| ATOM | 2243 | O | HOH | W | 141 | −14.593 | 45.948 | 11.031 | 1.00 | 34.46 | | O |
| ANISOU | 2243 | O | HOH | W | 141 | 4698 | 4941 | 3453 | −36 | −436 | −103 | O |
| ATOM | 2246 | O | HOH | W | 142 | 3.026 | 46.194 | 47.081 | 1.00 | 30.31 | | O |
| ANISOU | 2246 | O | HOH | W | 142 | 4040 | 3696 | 3779 | 396 | 127 | 314 | O |
| ATOM | 2249 | O | HOH | W | 143 | −10.257 | 62.436 | 22.662 | 1.00 | 27.49 | | O |
| ANISOU | 2249 | O | HOH | W | 143 | 4475 | 2149 | 3821 | −156 | 194 | 293 | O |
| ATOM | 2252 | O | HOH | W | 144 | −11.773 | 55.925 | 48.147 | 1.00 | 37.17 | | O |
| ANISOU | 2252 | O | HOH | W | 144 | 5025 | 4647 | 4450 | 163 | −83 | −304 | O |
| ATOM | 2255 | O | HOH | W | 145 | −5.114 | 46.241 | 13.538 | 1.00 | 49.22 | | O |
| ANISOU | 2255 | O | HOH | W | 145 | 6244 | 6477 | 5981 | −67 | −13 | −4 | O |
| ATOM | 2258 | O | HOH | W | 146 | −9.018 | 64.821 | 26.736 | 1.00 | 35.18 | | O |
| ANISOU | 2258 | O | HOH | W | 146 | 4216 | 4204 | 4947 | 226 | 158 | 3 | O |
| ATOM | 2261 | O | HOH | W | 148 | 0.002 | 42.809 | 21.295 | 0.33 | 20.62 | | O |
| ANISOU | 2261 | O | HOH | W | 148 | 2467 | 2467 | 2897 | 0 | 0 | 0 | O |
| ATOM | 2264 | O | HOH | W | 149 | −20.830 | 48.490 | 27.934 | 1.00 | 23.74 | | O |
| ANISOU | 2264 | O | HOH | W | 149 | 2862 | 3179 | 2979 | 95 | 171 | −613 | O |
| ATOM | 2267 | O | HOH | W | 150 | −14.581 | 41.607 | 22.420 | 1.00 | 23.84 | | O |
| ANISOU | 2267 | O | HOH | W | 150 | 3791 | 2062 | 3204 | −201 | −131 | −224 | O |
| ATOM | 2270 | O | HOH | W | 151 | −17.034 | 53.779 | 42.672 | 1.00 | 28.29 | | O |
| ANISOU | 2270 | O | HOH | W | 151 | 3875 | 3593 | 3279 | −242 | 449 | −161 | O |
| ATOM | 2273 | O | HOH | W | 153 | −0.167 | 61.931 | 34.026 | 1.00 | 32.11 | | O |
| ANISOU | 2273 | O | HOH | W | 153 | 4347 | 3624 | 4229 | −743 | −42 | 348 | O |
| ATOM | 2276 | O | HOH | W | 154 | −2.805 | 61.480 | 18.807 | 1.00 | 28.09 | | O |
| ANISOU | 2276 | O | HOH | W | 154 | 4258 | 2391 | 4021 | 85 | 10 | 886 | O |
| ATOM | 2279 | O | HOH | W | 155 | −14.660 | 57.218 | 18.936 | 1.00 | 36.02 | | O |
| ANISOU | 2279 | O | HOH | W | 155 | 4563 | 4355 | 4766 | 342 | −220 | 0 | O |
| ATOM | 2282 | O | HOH | W | 156 | −15.297 | 41.286 | 16.642 | 1.00 | 28.25 | | O |
| ANISOU | 2282 | O | HOH | W | 156 | 3304 | 3297 | 4131 | −545 | −518 | −216 | O |
| ATOM | 2285 | O | HOH | W | 157 | −2.243 | 57.376 | 49.840 | 1.00 | 27.49 | | O |
| ANISOU | 2285 | O | HOH | W | 157 | 3773 | 3402 | 3269 | 111 | −113 | −221 | O |
| ATOM | 2288 | O | HOH | W | 158 | −11.052 | 65.750 | 31.721 | 1.00 | 30.00 | | O |
| ANISOU | 2288 | O | HOH | W | 158 | 4377 | 2979 | 4041 | −36 | −73 | −204 | O |
| ATOM | 2291 | O | HOH | W | 159 | −18.733 | 50.460 | 31.261 | 1.00 | 32.59 | | O |
| ANISOU | 2291 | O | HOH | W | 159 | 3598 | 3681 | 5101 | −233 | 111 | −265 | O |
| ATOM | 2294 | O | HOH | W | 160 | −17.389 | 63.326 | 33.651 | 1.00 | 29.08 | | O |
| ANISOU | 2294 | O | HOH | W | 160 | 3061 | 3483 | 4505 | 731 | 254 | −527 | O |
| ATOM | 2297 | O | HOH | W | 163 | −2.824 | 41.746 | 36.954 | 1.00 | 22.98 | | O |
| ANISOU | 2297 | O | HOH | W | 163 | 3178 | 2376 | 3174 | −19 | 236 | 96 | O |
| ATOM | 2300 | O | HOH | W | 166 | −1.347 | 62.571 | 51.614 | 1.00 | 29.56 | | O |
| ANISOU | 2300 | O | HOH | W | 166 | 3997 | 3431 | 3803 | 224 | 80 | 305 | O |
| ATOM | 2303 | O | HOH | W | 167 | −17.508 | 64.604 | 37.456 | 1.00 | 38.73 | | O |
| ANISOU | 2303 | O | HOH | W | 167 | 5032 | 5154 | 4528 | 35 | 0 | −37 | O |
| ATOM | 2306 | O | HOH | W | 168 | −10.121 | 49.371 | 45.573 | 1.00 | 36.68 | | O |
| ANISOU | 2306 | O | HOH | W | 168 | 4890 | 4692 | 4353 | −254 | 63 | −212 | O |
| ATOM | 2309 | O | HOH | W | 169 | −17.875 | 59.214 | 31.890 | 1.00 | 39.88 | | O |
| ANISOU | 2309 | O | HOH | W | 169 | 5220 | 5114 | 4815 | 12 | −68 | −283 | O |
| ATOM | 2312 | O | HOH | W | 170 | −11.118 | 64.605 | 28.842 | 1.00 | 33.58 | | O |
| ANISOU | 2312 | O | HOH | W | 170 | 4482 | 4039 | 4238 | 135 | −163 | 336 | O |
| ATOM | 2315 | O | HOH | W | 171 | 2.773 | 57.449 | 35.259 | 1.00 | 38.57 | | O |
| ANISOU | 2315 | O | HOH | W | 171 | 4880 | 4868 | 4906 | −151 | 60 | −120 | O |
| ATOM | 2318 | O | HOH | W | 172 | −16.223 | 60.008 | 45.734 | 1.00 | 33.89 | | O |
| ANISOU | 2318 | O | HOH | W | 172 | 4171 | 4323 | 4381 | −442 | 77 | −118 | O |
| ATOM | 2321 | O | HOH | W | 173 | −17.917 | 64.871 | 51.915 | 1.00 | 40.44 | | O |
| ANISOU | 2321 | O | HOH | W | 173 | 4801 | 5476 | 5087 | 121 | 463 | −251 | O |
| ATOM | 2324 | O | HOH | W | 176 | −18.615 | 51.603 | 41.037 | 1.00 | 39.91 | | O |
| ANISOU | 2324 | O | HOH | W | 176 | 4865 | 5447 | 4850 | 9 | 126 | −181 | O |
| ATOM | 2327 | O | HOH | W | 177 | −2.290 | 43.463 | 17.960 | 1.00 | 36.57 | | O |
| ANISOU | 2327 | O | HOH | W | 177 | 5258 | 4396 | 4239 | −273 | 249 | 108 | O |
| ATOM | 2330 | O | HOH | W | 178 | −18.592 | 40.312 | 37.066 | 1.00 | 45.41 | | O |
| ANISOU | 2330 | O | HOH | W | 178 | 5988 | 5497 | 5766 | −270 | 217 | −149 | O |
| ATOM | 2333 | O | HOH | W | 179 | −12.661 | 50.011 | 44.964 | 1.00 | 38.21 | | O |
| ANISOU | 2333 | O | HOH | W | 179 | 5534 | 4642 | 4341 | −9 | 186 | −8 | O |
| ATOM | 2336 | O | HOH | W | 180 | −15.870 | 65.334 | 26.487 | 1.00 | 44.98 | | O |
| ANISOU | 2336 | O | HOH | W | 180 | 5920 | 5605 | 5562 | 238 | −30 | 71 | O |
| ATOM | 2339 | O | HOH | W | 181 | −1.497 | 45.718 | 42.268 | 1.00 | 46.67 | | O |
| ANISOU | 2339 | O | HOH | W | 181 | 6000 | 5242 | 6489 | 133 | −129 | 145 | O |
| ATOM | 2342 | O | HOH | W | 182 | −9.007 | 54.660 | 15.337 | 1.00 | 30.30 | | O |
| ANISOU | 2342 | O | HOH | W | 182 | 4148 | 3741 | 3624 | 312 | −591 | 636 | O |
| ATOM | 2345 | O | HOH | W | 183 | −16.725 | 68.640 | 50.051 | 1.00 | 35.36 | | O |
| ANISOU | 2345 | O | HOH | W | 183 | 4154 | 4188 | 5092 | −368 | 292 | 130 | O |
| ATOM | 2348 | O | HOH | W | 184 | −6.500 | 62.585 | 20.455 | 1.00 | 38.40 | | O |
| ANISOU | 2348 | O | HOH | W | 184 | 5138 | 4120 | 5332 | 162 | −451 | 215 | O |
| ATOM | 2351 | O | HOH | W | 185 | −0.818 | 63.914 | 35.532 | 1.00 | 42.56 | | O |
| ANISOU | 2351 | O | HOH | W | 185 | 5537 | 5392 | 5242 | −378 | 57 | 0 | O |
| ATOM | 2354 | O | HOH | W | 186 | −19.261 | 41.968 | 17.498 | 1.00 | 32.55 | | O |
| ANISOU | 2354 | O | HOH | W | 186 | 4279 | 3955 | 4131 | −433 | −664 | −360 | O |
| ATOM | 2357 | O | HOH | W | 187 | −2.161 | 54.751 | 12.443 | 1.00 | 51.33 | | O |
| ANISOU | 2357 | O | HOH | W | 187 | 6555 | 6548 | 6400 | −207 | 67 | −55 | O |
| ATOM | 2360 | O | HOH | W | 188 | 8.065 | 54.660 | 45.373 | 1.00 | 47.00 | | O |
| ANISOU | 2360 | O | HOH | W | 188 | 5848 | 5869 | 6138 | 67 | −16 | −106 | O |
| ATOM | 2363 | O | HOH | W | 190 | 8.082 | 49.232 | 46.085 | 1.00 | 41.83 | | O |
| ANISOU | 2363 | O | HOH | W | 190 | 5311 | 5032 | 5551 | 175 | −35 | 142 | O |
| ATOM | 2366 | O | HOH | W | 193 | −7.492 | 50.820 | 48.827 | 1.00 | 46.76 | | O |
| ANISOU | 2366 | O | HOH | W | 193 | 6076 | 5927 | 5762 | 53 | 179 | 130 | O |
| ATOM | 2369 | O | HOH | W | 195 | −13.334 | 68.992 | 41.155 | 1.00 | 48.46 | | O |
| ANISOU | 2369 | O | HOH | W | 195 | 6062 | 6228 | 6122 | 21 | 88 | 24 | O |
| ATOM | 2372 | O | HOH | W | 198 | −11.210 | 68.088 | 40.735 | 1.00 | 37.12 | | O |
| ANISOU | 2372 | O | HOH | W | 198 | 5096 | 4237 | 4768 | −23 | 68 | −255 | O |
| ATOM | 2375 | O | HOH | W | 199 | −1.209 | 45.229 | 45.850 | 1.00 | 41.80 | | O |
| ANISOU | 2375 | O | HOH | W | 199 | 5122 | 4864 | 5894 | 40 | 91 | −138 | O |
| ATOM | 2378 | O | HOH | W | 200 | −0.209 | 64.949 | 31.175 | 1.00 | 42.90 | | O |
| ANISOU | 2378 | O | HOH | W | 200 | 5637 | 5446 | 5217 | −258 | −149 | 127 | O |
| ATOM | 2381 | O | HOH | W | 201 | −14.698 | 49.311 | 46.572 | 1.00 | 43.90 | | O |
| ANISOU | 2381 | O | HOH | W | 201 | 5543 | 5559 | 5575 | −94 | −37 | −90 | O |
| ATOM | 2384 | O | HOH | W | 202 | −5.161 | 61.193 | 17.491 | 1.00 | 39.45 | | O |
| ANISOU | 2384 | O | HOH | W | 202 | 5371 | 4486 | 5130 | −174 | −36 | 376 | O |
| ATOM | 2387 | O | HOH | W | 206 | −3.300 | 66.537 | 37.955 | 1.00 | 45.17 | | O |
| ANISOU | 2387 | O | HOH | W | 206 | 5318 | 5743 | 6101 | −155 | 41 | −79 | O |
| ATOM | 2390 | O | HOH | W | 207 | −20.335 | 50.000 | 35.063 | 1.00 | 46.13 | | O |
| ANISOU | 2390 | O | HOH | W | 207 | 5544 | 5707 | 6276 | −234 | −196 | 17 | O |
| ATOM | 2393 | O | HOH | W | 208 | −21.010 | 60.974 | 28.216 | 1.00 | 44.88 | | O |
| ANISOU | 2393 | O | HOH | W | 208 | 5585 | 5720 | 5747 | −87 | −138 | 72 | O |
| ATOM | 2396 | O | HOH | W | 209 | −23.773 | 41.837 | 41.420 | 1.00 | 49.48 | | O |
| ANISOU | 2396 | O | HOH | W | 209 | 6015 | 6280 | 6505 | 1 | −16 | 63 | O |
| ATOM | 2399 | O | HOH | W | 210 | 0.002 | 42.809 | 47.889 | 0.33 | 43.16 | | O |
| ANISOU | 2399 | O | HOH | W | 210 | 5469 | 5469 | 5460 | 0 | 0 | 0 | O |
| ATOM | 2402 | O | HOH | W | 211 | −0.940 | 65.973 | 39.157 | 1.00 | 35.55 | | O |
| ANISOU | 2402 | O | HOH | W | 211 | 5140 | 4032 | 4332 | −152 | −223 | −32 | O |
| ATOM | 2405 | O | HOH | W | 212 | 1.186 | 64.588 | 38.091 | 1.00 | 41.23 | | O |
| ANISOU | 2405 | O | HOH | W | 212 | 5243 | 4868 | 5551 | −229 | 10 | 97 | O |
| ATOM | 2408 | O | HOH | W | 213 | −20.041 | 61.023 | 38.988 | 1.00 | 44.93 | | O |
| ANISOU | 2408 | O | HOH | W | 213 | 5638 | 5829 | 5603 | −30 | −143 | −209 | O |
| ATOM | 2411 | O | HOH | W | 214 | −20.170 | 55.338 | 15.170 | 1.00 | 51.70 | | O |
| ANISOU | 2411 | O | HOH | W | 214 | 6589 | 6588 | 6465 | −21 | −68 | 79 | O |
| ATOM | 2414 | O | HOH | W | 215 | −17.144 | 41.072 | 21.849 | 1.00 | 39.18 | | O |
| ANISOU | 2414 | O | HOH | W | 215 | 5000 | 5091 | 4795 | 74 | 17 | −154 | O |
| ATOM | 2417 | O | HOH | W | 216 | −4.308 | 61.704 | 46.630 | 1.00 | 42.61 | | O |
| ANISOU | 2417 | O | HOH | W | 216 | 5390 | 5469 | 5330 | 2 | 241 | −264 | O |
| ATOM | 2420 | O | HOH | W | 217 | −8.078 | 69.110 | 44.390 | 1.00 | 37.23 | | O |
| ANISOU | 2420 | O | HOH | W | 217 | 4457 | 4659 | 5029 | −81 | 119 | 289 | O |
| ATOM | 2423 | O | HOH | W | 218 | 3.813 | 55.462 | 30.287 | 1.00 | 20.17 | | O |
| ANISOU | 2423 | O | HOH | W | 218 | 3277 | 2333 | 2054 | 957 | 163 | 233 | O |
| ATOM | 2426 | O | HOH | W | 219 | 5.588 | 55.956 | 29.777 | 1.00 | 18.62 | | O |
| ANISOU | 2426 | O | HOH | W | 219 | 2809 | 1528 | 2737 | −241 | −909 | 182 | O |
| ATOM | 2429 | O | HOH | W | 220 | 4.873 | 57.326 | 31.456 | 1.00 | 33.95 | | O |
| ANISOU | 2429 | O | HOH | W | 220 | 3799 | 4714 | 4383 | 1 | −56 | −174 | O |
| ATOM | 2432 | O | HOH | W | 221 | −2.690 | 43.167 | 45.639 | 1.00 | 35.10 | | O |
| ANISOU | 2432 | O | HOH | W | 221 | 4700 | 4210 | 4426 | 39 | −39 | 53 | O |
| ATOM | 2435 | O | HOH | W | 222 | −6.453 | 68.695 | 30.899 | 1.00 | 46.25 | | O |
| ANISOU | 2435 | O | HOH | W | 222 | 6040 | 5876 | 5656 | 167 | 16 | −143 | O |

TABLE 9

| | | |
|---|---|---|
| HEADER | ---- | XX-XXX-XX xxxx |
| COMPND | --- | |

| | | | |
|---|---|---|---|
| REMARK | 3 | | |
| REMARK | 3 | REFINEMENT. | |
| REMARK | 3 | PROGRAM | : REFMAC 5.2.0005 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.00 |
| REMARK | 3 | RESOLUTION RANGE LOW | (ANGSTROMS) | : 30.00 |
| REMARK | 3 | DATA CUTOFF | (SIGMA(F)) | : NONE |

| | | | | |
|---|---|---|---|---|
| REMARK | 3 | COMPLETENESS FOR RANGE | (%) | : 98.82 |
| REMARK | 3 | NUMBER OF REFLECTIONS | | : 60840 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.22320 |
| REMARK | 3 | R VALUE | (WORKING SET) | : 0.22054 |
| REMARK | 3 | FREE R VALUE | | : 0.24713 |

| | | | | |
|---|---|---|---|---|
| REMARK | 3 | FREE R VALUE TEST SET SIZE | (%) | : 10.1 |
| REMARK | 3 | FREE R VALUE TEST SET COUNT | | : 6853 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 25 |
| REMARK | 3 | BIN RESOLUTION RANGE HIGH | | : 2.004 |
| REMARK | 3 | BIN RESOLUTION RANGE LOW | | : 2.045 |
| REMARK | 3 | REFLECTION IN BIN | (WORKING SET) | : 3505 |
| REMARK | 3 | BIN COMPLETENESS | (WORKING + TEST) (%) | : 97.12 |
| REMARK | 3 | BIN R VALUE | (WORKING SET) | : 0.236 |

| | | | |
|---|---|---|---|
| REMARK | 3 | BIN FREE R VALUE SET COUNT | : 374 |
| REMARK | 3 | BIN FREE R VALUE | : 0.293 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 4323 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : 34.815 |

| REMARK | 3 | OVERALL ANISOTROPIC B VALUE. | |
|---|---|---|---|
| REMARK | 3 | B11 (A**2) : | 1.10 |
| REMARK | 3 | B22 (A**2) : | 1.10 |
| REMARK | 3 | B33 (A**2) : | −1.65 |
| REMARK | 3 | B12 (A**2) : | 0.55 |
| REMARK | 3 | B13 (A**2) : | 0.00 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.145 |
| REMARK | 3 | ESU BASED ON FREE R VALUE | (A): | 0.137 |
| REMARK | 3 | ESU BASED ON MAXIMUM LIKELIHOOD | (A): | 0.088 |
| REMARK | 3 | ESU FOR B VALUES BASED ON MAXIMUM LIKELIHOOD | (A**2): | 5.995 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.941 |
| REMARK | 3 | CORRELATION COEFFICIENT FO-FC FREE | : 0.927 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 4259 ; | 0.007 ; | 0.022 |
| REMARK | 3 | BOND LENGTHS OTHERS | (A): | 3663 ; | 0.001 ; | 0.020 |
| REMARK | 3 | BOND ANGLES REFINED ATOMS | (DEGREES): | 5799 ; | 1.041 ; | 1.958 |
| REMARK | 3 | BOND ANGLES OTHERS | (DEGREES): | 8587 ; | 0.692 ; | 3.000 |
| REMARK | 3 | TORSION ANGLES, PERIOD 1 | (DEGREES): | 537 ; | 5.786 ; | 5.000 |
| REMARK | 3 | TORSION ANGLES, PERIOD 2 | (DEGREES): | 192 ; | 35.940 ; | 24.010 |
| REMARK | 3 | TORSION ANGLES, PERIOD 3 | (DEGREES): | 668 ; | 13.371 ; | 15.000 |
| REMARK | 3 | TORSION ANGLES, PERIOD 4 | (DEGREES): | 30 ; | 14.820 ; | 15.000 |
| REMARK | 3 | CHIRAL-CENTER RESTRAINTS | (A**3): | 626 ; | 0.067 ; | 0.200 |

| | | | | | | |
|---|---|---|---|---|---|---|
| REMARK | 3 | GENERAL PLANES REFINED ATOMS | (A): | 4781 ; | 0.003 ; | 0.020 |
| REMARK | 3 | GENERAL PLANES OTHERS | (A): | 825 ; | 0.001 ; | 0.020 |
| REMARK | 3 | NON-BONDED CONTACTS REFINED ATOMS | (A): | 672 ; | 0.189 ; | 0.200 |
| REMARK | 3 | NON-BONDED CONTACTS OTHERS | (A): | 3395 ; | 0.173 ; | 0.200 |
| REMARK | 3 | NON-BONDED TORSION REFINED ATOMS | (A): | 1977 ; | 0.167 ; | 0.200 |
| REMARK | 3 | NON-BONDED TORSION OTHERS | (A): | 2518 ; | 0.078 ; | 0.200 |
| REMARK | 3 | H-BOND (X...Y) REFINED ATOMS | (A): | 168 ; | 0.118 ; | 0.200 |
| REMARK | 3 | SYMMETRY VDW REFINED ATOMS | (A): | 12 ; | 0.110 ; | 0.200 |
| REMARK | 3 | SYMMETRY VDW OTHERS | (A): | 89 ; | 0.173 ; | 0.200 |
| REMARK | 3 | SYMMETRY H-BOND REFINED ATOMS | (A): | 14 ; | 0.120 ; | 0.200 |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 3484 ; | 2.469 ; | 2.500 |
| REMARK | 3 | MAIN-CHAIN BOND OTHER ATOMS | (A**2): | 1087 ; | 0.398 ; | 2.500 |
| REMARK | 3 | MAIN-CHAIN ANGLE REFINED ATOMS | (A**2): | 4384 ; | 3.073 ; | 5.000 |
| REMARK | 3 | SIDE-CHAIN BOND REFINED ATOMS | (A**2): | 1787 ; | 2.335 ; | 2.500 |
| REMARK | 3 | SIDE-CHAIN ANGLE REFINED ATOMS | (A**2): | 1415 ; | 3.346 ; | 5.000 |
| REMARK | 3 | | | | | |
| REMARK | 3 | NCS RESTRAINTS STATISTICS | | | | |
| REMARK | 3 | NUMBER OF NCS GROUPS: NULL | | | | |
| REMARK | 3 | | | | | |
| REMARK | 3 | | | | | |

| REMARK | 3 | TLS DETAILS | | | | | |
|---|---|---|---|---|---|---|---|
| REMARK | 3 | NUMBER OF TLS GROUPS : 8 | | | | | |
| REMARK | 3 | ATOM RECORD CONTAINS RESIDUAL B FACTORS ONLY | | | | | |
| REMARK | 3 | | | | | | |
| REMARK | 3 | TLS GROUP : 1 | | | | | |
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | F | 57 | | F | 191 |

| REMARK | 3 | ORIGIN FOR THE GROUP (A): 43.9142 102.7918 174.8679 | | | | | |
|---|---|---|---|---|---|---|---|
| REMARK | 3 | T TENSOR | | | | | |
| REMARK | 3 | T11: | −0.0988 | T22: | −0.1296 | | |
| REMARK | 3 | T33: | −0.0612 | T12: | 0.0227 | | |
| REMARK | 3 | T13: | 0.0310 | T23: | 0.0020 | | |
| REMARK | 3 | L TENSOR | | | | | |
| REMARK | 3 | L11: | 1.4095 | L22: | 2.0527 | | |
| REMARK | 3 | L33: | 1.6573 | L12: | 0.4989 | | |
| REMARK | 3 | L13: | 0.4289 | L23: | 0.5244 | | |
| REMARK | 3 | S TENSOR | | | | | |
| REMARK | 3 | S11: | 0.0610 | S12: | −0.1507 | S13: | 0.2233 |
| REMARK | 3 | S21: | 0.1403 | S22: | −0.0410 | S23: | 0.1536 |
| REMARK | 3 | S31: | −0.2464 | S32: | −0.2021 | S33: | −0.0199 |
| REMARK | 3 | | | | | | |

| REMARK | 3 | TLS GROUP : 2 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | G | 57 | | G | 191 |

| REMARK | 3 | ORIGIN FOR THE GROUP (A): 14.6102 58.4872 216.4521 | | | | | |
|---|---|---|---|---|---|---|---|
| REMARK | 3 | T TENSOR | | | | | |
| REMARK | 3 | T11: | 0.0055 | T22: | 0.0600 | | |
| REMARK | 3 | T33: | −0.0638 | T12: | 0.0168 | | |
| REMARK | 3 | T13: | −0.0847 | T23: | −0.0274 | | |
| REMARK | 3 | L TENSOR | | | | | |
| REMARK | 3 | L11: | 1.6896 | L22: | 2.6344 | | |
| REMARK | 3 | L33: | 2.0297 | L12: | −0.0786 | | |
| REMARK | 3 | L13: | 0.1509 | L23: | −0.4868 | | |
| REMARK | 3 | S TENSOR | | | | | |
| REMARK | 3 | S11: | 0.0348 | S12: | −0.3010 | S13: | −0.0765 |
| REMARK | 3 | S21: | 0.4486 | S22: | 0.0609 | S23: | −0.3884 |
| REMARK | 3 | S31: | 0.0253 | S32: | 0.3677 | S33: | −0.0957 |
| REMARK | 3 | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | R | 29 | | R | 108 |

| REMARK | 3 | ORIGIN FOR THE GROUP (A): 25.6686 87.7196 169.9556 | | | | | |
|---|---|---|---|---|---|---|---|
| REMARK | 3 | T TENSOR | | | | | |
| REMARK | 3 | T11: | −0.1381 | T22: | −0.0932 | | |
| REMARK | 3 | T33: | −0.0595 | T12: | −0.0493 | | |
| REMARK | 3 | T13: | −0.0193 | T23: | −0.0313 | | |
| REMARK | 3 | L TENSOR | | | | | |
| REMARK | 3 | L11: | 1.5922 | L22: | 0.6020 | | |
| REMARK | 3 | L33: | 5.1978 | L12: | 0.4407 | | |
| REMARK | 3 | L13: | −0.7141 | L23: | −0.8882 | | |
| REMARK | 3 | S TENSOR | | | | | |
| REMARK | 3 | S11: | −0.0233 | S12: | 0.0770 | S13: | 0.1890 |
| REMARK | 3 | S21: | 0.0376 | S22: | −0.0632 | S23: | 0.1629 |
| REMARK | 3 | S31: | −0.1226 | S32: | −0.1850 | S33: | 0.0865 |
| REMARK | 3 | | | | | | |

| REMARK | 3 | TLS GROUP : 4 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | R | 109 | | R | 140 |

| REMARK | 3 | ORIGIN FOR THE GROUP (A): 25.3215 93.6141 196.7325 | | | | | |
|---|---|---|---|---|---|---|---|
| REMARK | 3 | T TENSOR | | | | | |
| REMARK | 3 | T11: | 0.1474 | T22: | 0.1342 | | |
| REMARK | 3 | T33: | 0.0065 | T12: | −0.2101 | | |
| REMARK | 3 | T13: | 0.1620 | T23: | −0.1749 | | |
| REMARK | 3 | L TENSOR | | | | | |
| REMARK | 3 | L11: | 8.3007 | L22: | 4.6110 | | |
| REMARK | 3 | L33: | 6.6462 | L12: | −0.4453 | | |
| REMARK | 3 | L13: | −5.4694 | L23: | 1.1369 | | |
| REMARK | 3 | S TENSOR | | | | | |
| REMARK | 3 | S11: | 0.7103 | S12: | −0.8209 | S13: | 0.9584 |
| REMARK | 3 | S21: | 0.1917 | S22: | −0.0805 | S23: | 0.0379 |
| REMARK | 3 | S31: | −1.0716 | S32: | 0.5838 | S33: | −0.6298 |
| REMARK | 3 | | | | | | |

| REMARK | 3 | TLS GROUP : 5 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | R | 141 | | R | 166 |

| REMARK | 3 | ORIGIN FOR THE GROUP (A): 16.8496 100.8594 213.0094 | | | | | |
|---|---|---|---|---|---|---|---|
| REMARK | 3 | T TENSOR | | | | | |
| REMARK | 3 | T11: | 0.4924 | T22: | 0.2599 | | |
| REMARK | 3 | T33: | 0.1608 | T12: | −0.1013 | | |
| REMARK | 3 | T13: | 0.2358 | T23: | −0.2032 | | |
| REMARK | 3 | L TENSOR | | | | | |
| REMARK | 3 | L11: | 5.4047 | L22: | 9.7294 | | |
| REMARK | 3 | L33: | 22.1208 | L12: | −3.3609 | | |
| REMARK | 3 | L13: | −7.1965 | L23: | 5.4286 | | |
| REMARK | 3 | S TENSOR | | | | | |
| REMARK | 3 | S11: | −0.0919 | S12: | −0.2657 | S13: | 0.4059 |
| REMARK | 3 | S21: | 0.5516 | S22: | 0.4574 | S23: | 0.6014 |
| REMARK | 3 | S31: | −1.2713 | S32: | 0.1665 | S33: | −0.3654 |
| REMARK | 3 | | | | | | |

| REMARK | 3 | TLS GROUP : 6 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | T | 29 | | T | 108 |

| REMARK | 3 | ORIGIN FOR THE GROUP (A): 16.2781 81.5721 211.5454 | | | | | |
|---|---|---|---|---|---|---|---|
| REMARK | 3 | T TENSOR | | | | | |
| REMARK | 3 | T11: | 0.1643 | T22: | 0.0992 | | |
| REMARK | 3 | T33: | 0.0653 | T12: | −0.1703 | | |
| REMARK | 3 | T13: | 0.0085 | T23: | −0.0648 | | |
| REMARK | 3 | L TENSOR | | | | | |
| REMARK | 3 | L11: | 1.5274 | L22: | 0.8567 | | |
| REMARK | 3 | L33: | 8.6339 | L12: | −0.2123 | | |
| REMARK | 3 | L13: | −2.3410 | L23: | −0.0957 | | |
| REMARK | 3 | S TENSOR | | | | | |
| REMARK | 3 | S11: | 0.1399 | S12: | −0.3521 | S13: | 0.1940 |
| REMARK | 3 | S21: | 0.3353 | S22: | −0.0519 | S23: | −0.2094 |
| REMARK | 3 | S31: | −0.7345 | S32: | 0.7190 | S33: | −0.0880 |
| REMARK | 3 | | | | | | |

| REMARK | 3 | TLS GROUP : 7 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | T | 109 | | T | 140 |

| REMARK | 3 | ORIGIN FOR THE GROUP (A): 18.2096 78.1285 240.9816 | | | | | |
|---|---|---|---|---|---|---|---|
| REMARK | 3 | T TENSOR | | | | | |
| REMARK | 3 | T11: | 0.4902 | T22: | 0.3530 | | |
| REMARK | 3 | T33: | 0.0272 | T12: | −0.1840 | | |
| REMARK | 3 | T13: | −0.2380 | T23: | −0.4636 | | |
| REMARK | 3 | L TENSOR | | | | | |
| REMARK | 3 | L11: | 3.2040 | L22: | 4.6424 | | |
| REMARK | 3 | L33: | 38.8550 | L12: | −1.8483 | | |
| REMARK | 3 | L13: | 10.3445 | L23: | −1.5503 | | |
| REMARK | 3 | S TENSOR | | | | | |
| REMARK | 3 | S11: | −0.3552 | S12: | 0.6662 | S13: | −0.7694 |
| REMARK | 3 | S21: | 0.2239 | S22: | 0.7654 | S23: | −0.5121 |
| REMARK | 3 | S31: | 0.7250 | S32: | 2.1475 | S33: | −0.4102 |
| REMARK | 3 | | | | | | |

| REMARK | 3 | TLS GROUP : 8 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | T | 141 | | T | 166 |

| REMARK | 3 | ORIGIN FOR THE GROUP (A): 26.4654 81.7957 256.4784 | | | | | |
|---|---|---|---|---|---|---|---|
| REMARK | 3 | T TENSOR | | | | | |
| REMARK | 3 | T11: | 0.4382 | T22: | 0.4983 | | |
| REMARK | 3 | T33: | 0.2746 | T12: | 0.1032 | | |
| REMARK | 3 | T13: | 0.0217 | T23: | −0.3335 | | |
| REMARK | 3 | L TENSOR | | | | | |
| REMARK | 3 | L11: | 27.7115 | L22: | 8.6242 | | |
| REMARK | 3 | L33: | 32.0119 | L12: | 0.0615 | | |
| REMARK | 3 | L13: | 13.4817 | L23: | 2.4624 | | |
| REMARK | 3 | S TENSOR | | | | | |
| REMARK | 3 | S11: | 0.0981 | S12: | 0.2481 | S13: | −0.3615 |
| REMARK | 3 | S21: | −0.1364 | S22: | 0.4003 | S23: | −0.0823 |
| REMARK | 3 | S31: | 0.2206 | S32: | 1.9882 | S33: | −0.4983 |
| REMARK | 3 | | | | | | |
| REMARK | 3 | | | | | | |

| REMARK | 3 | BULK SOLVENT MODELLING. | | | | | |
|---|---|---|---|---|---|---|---|
| REMARK | 3 | METHOD USED : MASK | | | | | |
| REMARK | 3 | PARAMETERS FOR MASK CALCULATION | | | | | |
| REMARK | 3 | VDW PROBE RADIUS | | | | | : 1.40 |
| REMARK | 3 | ION PROBE RADIUS | | | | | : 0.80 |
| REMARK | 3 | SHRINKAGE RADIUS | | | | | : 0.80 |
| REMARK | 3 | | | | | | |
| REMARK | 3 | OTHER REFINEMENT REMARKS: | | | | | |
| REMARK | 3 | HYDROGENS HAVE BEEN ADDED IN THE RIDING POSITIONS | | | | | |
| REMARK | 3 | | | | | | |
| SSBOND | 1 | CYS | F | 70 | CYS | F | 163 |
| SSBOND | 2 | CYS | F | 98 | CYS | F | 183 |
| SSBOND | 3 | CYS | G | 70 | CYS | G | 163 |
| SSBOND | 4 | CYS | G | 98 | CYS | G | 183 |
| SSBOND | 5 | CYS | R | 31 | CYS | R | 42 |
| SSBOND | 6 | CYS | R | 43 | CYS | R | 56 |
| SSBOND | 7 | CYS | R | 46 | CYS | R | 64 |
| SSBOND | 8 | CYS | R | 67 | CYS | R | 81 |
| SSBOND | 9 | CYS | R | 87 | CYS | R | 107 |
| SSBOND | 10 | CYS | R | 109 | CYS | R | 125 |
| SSBOND | 11 | CYS | R | 128 | CYS | R | 141 |
| SSBOND | 12 | CYS | T | 31 | CYS | T | 42 |
| SSBOND | 13 | CYS | T | 43 | CYS | T | 56 |
| SSBOND | 14 | CYS | T | 46 | CYS | T | 64 |
| SSBOND | 15 | CYS | T | 67 | CYS | T | 81 |
| SSBOND | 16 | CYS | T | 87 | CYS | T | 107 |
| SSBOND | 17 | CYS | T | 109 | CYS | T | 125 |

| | | | |
|---|---|---|---|
| LINK | ALA T 150 | THR T 154 | gap |

| | |
|---|---|
| CRYST1 | 104.729 104.729 478.161 90.00 90.00 120.00 H 3 2 |

| | | | | |
|---|---|---|---|---|
| SCALE1 | 0.009548 | 0.005513 | 0.000000 | 0.00000 |
| SCALE2 | 0.000000 | 0.011026 | 0.000000 | 0.00000 |
| SCALE3 | 0.000000 | 0.000000 | 0.002091 | 0.00000 |

| | | | | | | | | | | | |
|---|---|---|---|---|---|---|---|---|---|---|---|
| ATOM | 1 | N | ASP | F | 57 | 44.153 | 95.412 | 151.972 | 1.00 | 66.84 | N |
| ATOM | 2 | CA | ASP | F | 57 | 44.513 | 96.691 | 151.280 | 1.00 | 62.49 | C |
| ATOM | 4 | CB | ASP | F | 57 | 43.323 | 97.226 | 150.460 | 1.00 | 66.77 | C |
| ATOM | 7 | CG | ASP | F | 57 | 43.491 | 98.680 | 150.054 | 1.00 | 68.45 | C |
| ATOM | 8 | OD1 | ASP | F | 57 | 44.502 | 99.002 | 149.389 | 1.00 | 69.74 | O |
| ATOM | 9 | OD2 | ASP | F | 57 | 42.604 | 99.496 | 150.396 | 1.00 | 69.38 | O |
| ATOM | 10 | C | ASP | F | 57 | 45.060 | 97.750 | 152.255 | 1.00 | 59.71 | C |
| ATOM | 11 | O | ASP | F | 57 | 46.158 | 98.267 | 152.040 | 1.00 | 58.36 | O |
| ATOM | 15 | N | PRO | F | 58 | 44.308 | 98.078 | 153.327 | 1.00 | 55.23 | N |
| ATOM | 16 | CA | PRO | F | 58 | 44.851 | 99.063 | 154.268 | 1.00 | 50.43 | C |
| ATOM | 18 | CB | PRO | F | 58 | 43.685 | 99.305 | 155.240 | 1.00 | 52.41 | C |
| ATOM | 21 | CG | PRO | F | 58 | 42.472 | 98.806 | 154.519 | 1.00 | 55.15 | C |
| ATOM | 24 | CD | PRO | F | 58 | 42.971 | 97.628 | 153.750 | 1.00 | 56.20 | C |
| ATOM | 27 | C | PRO | F | 58 | 46.086 | 98.539 | 155.010 | 1.00 | 42.75 | C |
| ATOM | 28 | O | PRO | F | 58 | 46.320 | 97.332 | 155.035 | 1.00 | 38.16 | O |
| ATOM | 29 | N | PRO | F | 59 | 46.877 | 99.445 | 155.610 | 1.00 | 38.23 | N |
| ATOM | 30 | CA | PRO | F | 59 | 48.073 | 99.007 | 156.317 | 1.00 | 36.33 | C |
| ATOM | 32 | CB | PRO | F | 59 | 48.828 | 100.312 | 156.568 | 1.00 | 37.32 | C |
| ATOM | 35 | CG | PRO | F | 59 | 47.768 | 101.348 | 156.639 | 1.00 | 38.40 | C |
| ATOM | 38 | CD | PRO | F | 59 | 46.688 | 100.902 | 155.689 | 1.00 | 37.89 | C |
| ATOM | 41 | C | PRO | F | 59 | 47.717 | 98.328 | 157.638 | 1.00 | 33.39 | C |
| ATOM | 42 | O | PRO | F | 59 | 46.563 | 98.381 | 158.075 | 1.00 | 30.88 | O |
| ATOM | 43 | N | ILE | F | 60 | 48.705 | 97.699 | 158.264 | 1.00 | 30.85 | N |
| ATOM | 44 | CA | ILE | F | 60 | 48.537 | 97.170 | 159.610 | 1.00 | 30.27 | C |
| ATOM | 46 | CB | ILE | F | 60 | 49.835 | 96.481 | 160.126 | 1.00 | 30.92 | C |
| ATOM | 48 | CG1 | ILE | F | 60 | 50.147 | 95.243 | 159.266 | 1.00 | 33.39 | C |
| ATOM | 51 | CD1 | ILE | F | 60 | 51.526 | 94.640 | 159.499 | 1.00 | 32.58 | C |
| ATOM | 55 | CG2 | ILE | F | 60 | 49.680 | 96.093 | 161.588 | 1.00 | 30.54 | C |
| ATOM | 59 | C | ILE | F | 60 | 48.131 | 98.349 | 160.497 | 1.00 | 28.20 | C |
| ATOM | 60 | O | ILE | F | 60 | 48.730 | 99.429 | 160.437 | 1.00 | 28.16 | O |
| ATOM | 62 | N | GLN | F | 61 | 47.094 | 98.134 | 161.291 | 1.00 | 28.70 | N |
| ATOM | 63 | CA | GLN | F | 61 | 46.498 | 99.182 | 162.107 | 1.00 | 29.25 | C |
| ATOM | 65 | CB | GLN | F | 61 | 45.046 | 98.826 | 162.445 | 1.00 | 28.25 | C |
| ATOM | 68 | CG | GLN | F | 61 | 44.132 | 98.713 | 161.225 | 1.00 | 28.67 | C |
| ATOM | 71 | CD | GLN | F | 61 | 43.855 | 100.050 | 160.579 | 1.00 | 29.94 | C |
| ATOM | 72 | OE1 | GLN | F | 61 | 43.066 | 100.834 | 161.098 | 1.00 | 30.67 | O |
| ATOM | 73 | NE2 | GLN | F | 61 | 44.500 | 100.322 | 159.439 | 1.00 | 30.01 | N |
| ATOM | 76 | C | GLN | F | 61 | 47.318 | 99.369 | 163.374 | 1.00 | 28.57 | C |
| ATOM | 77 | O | GLN | F | 61 | 47.563 | 98.415 | 164.118 | 1.00 | 30.22 | O |
| ATOM | 79 | N | ARG | F | 62 | 47.773 | 100.593 | 163.608 | 1.00 | 28.86 | N |
| ATOM | 80 | CA | ARG | F | 62 | 48.503 | 100.883 | 164.827 | 1.00 | 31.60 | C |
| ATOM | 82 | CB | ARG | F | 62 | 49.966 | 100.474 | 164.729 | 1.00 | 37.58 | C |
| ATOM | 85 | CG | ARG | F | 62 | 50.757 | 101.167 | 163.685 | 1.00 | 37.73 | C |
| ATOM | 88 | CD | ARG | F | 62 | 52.121 | 101.579 | 164.198 | 1.00 | 45.17 | C |
| ATOM | 91 | NE | ARG | F | 62 | 52.091 | 102.810 | 164.981 | 1.00 | 45.40 | N |
| ATOM | 93 | CZ | ARG | F | 62 | 53.165 | 103.490 | 165.380 | 1.00 | 45.42 | C |
| ATOM | 94 | NH1 | ARG | F | 62 | 54.384 | 103.090 | 165.071 | 1.00 | 52.09 | N |
| ATOM | 97 | NH2 | ARG | F | 62 | 53.018 | 104.608 | 166.080 | 1.00 | 47.32 | N |
| ATOM | 100 | C | ARG | F | 62 | 48.416 | 102.314 | 165.287 | 1.00 | 28.16 | C |
| ATOM | 101 | O | ARG | F | 62 | 48.334 | 103.261 | 164.488 | 1.00 | 29.23 | O |
| ATOM | 103 | N | LEU | F | 63 | 48.466 | 102.454 | 166.605 | 1.00 | 31.40 | N |
| ATOM | 104 | CA | LEU | F | 63 | 48.422 | 103.755 | 167.243 | 1.00 | 32.49 | C |
| ATOM | 106 | CB | LEU | F | 63 | 46.992 | 104.144 | 167.607 | 1.00 | 37.07 | C |
| ATOM | 109 | CG | LEU | F | 63 | 46.960 | 105.559 | 168.213 | 1.00 | 38.21 | C |
| ATOM | 111 | CD1 | LEU | F | 63 | 46.062 | 106.465 | 167.432 | 1.00 | 43.35 | C |
| ATOM | 115 | CD2 | LEU | F | 63 | 46.626 | 105.538 | 169.695 | 1.00 | 38.93 | C |
| ATOM | 119 | C | LEU | F | 63 | 49.264 | 103.731 | 168.496 | 1.00 | 29.85 | C |
| ATOM | 120 | O | LEU | F | 63 | 49.171 | 102.813 | 169.292 | 1.00 | 30.53 | O |
| ATOM | 122 | N | ARG | F | 64 | 50.072 | 104.764 | 168.655 | 1.00 | 29.53 | N |
| ATOM | 123 | CA | ARG | F | 64 | 50.780 | 105.013 | 169.891 | 1.00 | 29.90 | C |
| ATOM | 125 | CB | ARG | F | 64 | 52.272 | 104.954 | 169.622 | 1.00 | 28.03 | C |
| ATOM | 128 | CG | ARG | F | 64 | 53.133 | 105.132 | 170.831 | 1.00 | 32.92 | C |
| ATOM | 131 | CD | ARG | F | 64 | 54.559 | 104.905 | 170.420 | 1.00 | 37.46 | C |
| ATOM | 134 | NE | ARG | F | 64 | 55.483 | 105.430 | 171.397 | 1.00 | 44.60 | N |
| ATOM | 136 | CZ | ARG | F | 64 | 56.150 | 106.574 | 171.300 | 1.00 | 38.97 | C |
| ATOM | 137 | NH1 | ARG | F | 64 | 56.060 | 107.371 | 170.242 | 1.00 | 43.86 | N |
| ATOM | 140 | NH2 | ARG | F | 64 | 56.948 | 106.913 | 172.286 | 1.00 | 41.09 | N |
| ATOM | 143 | C | ARG | F | 64 | 50.384 | 106.395 | 170.392 | 1.00 | 29.14 | C |
| ATOM | 144 | O | ARG | F | 64 | 50.368 | 107.358 | 169.619 | 1.00 | 29.42 | O |
| ATOM | 146 | N | GLY | F | 65 | 50.066 | 106.485 | 171.682 | 1.00 | 31.17 | N |
| ATOM | 147 | CA | GLY | F | 65 | 49.743 | 107.761 | 172.320 | 1.00 | 31.68 | C |
| ATOM | 150 | C | GLY | F | 65 | 50.371 | 107.914 | 173.689 | 1.00 | 31.45 | C |
| ATOM | 151 | O | GLY | F | 65 | 50.594 | 106.928 | 174.405 | 1.00 | 33.45 | O |
| ATOM | 153 | N | ALA | F | 66 | 50.676 | 109.154 | 174.052 | 1.00 | 31.26 | N |
| ATOM | 154 | CA | ALA | F | 66 | 51.170 | 109.460 | 175.386 | 1.00 | 30.85 | C |
| ATOM | 156 | CB | ALA | F | 66 | 52.023 | 110.720 | 175.379 | 1.00 | 32.87 | C |
| ATOM | 160 | C | ALA | F | 66 | 49.988 | 109.620 | 176.340 | 1.00 | 33.24 | C |
| ATOM | 161 | O | ALA | F | 66 | 48.892 | 110.015 | 175.936 | 1.00 | 31.23 | O |
| ATOM | 163 | N | VAL | F | 67 | 50.236 | 109.295 | 177.606 | 1.00 | 35.76 | N |
| ATOM | 164 | CA | VAL | F | 67 | 49.267 | 109.460 | 178.688 | 1.00 | 35.90 | C |
| ATOM | 166 | CB | VAL | F | 67 | 48.763 | 108.080 | 179.201 | 1.00 | 34.68 | C |
| ATOM | 168 | CG1 | VAL | F | 67 | 47.646 | 108.253 | 180.228 | 1.00 | 36.61 | C |
| ATOM | 172 | CG2 | VAL | F | 67 | 48.271 | 107.241 | 178.034 | 1.00 | 35.63 | C |
| ATOM | 176 | C | VAL | F | 67 | 49.963 | 110.259 | 179.800 | 1.00 | 35.16 | C |
| ATOM | 177 | O | VAL | F | 67 | 50.901 | 109.777 | 180.440 | 1.00 | 36.77 | O |
| ATOM | 179 | N | THR | F | 68 | 49.541 | 111.504 | 179.980 | 1.00 | 38.98 | N |
| ATOM | 180 | CA | THR | F | 68 | 50.186 | 112.427 | 180.923 | 1.00 | 39.17 | C |
| ATOM | 182 | CB | THR | F | 68 | 50.799 | 113.635 | 180.147 | 1.00 | 43.00 | C |
| ATOM | 184 | OG1 | THR | F | 68 | 51.721 | 113.153 | 179.157 | 1.00 | 46.80 | O |
| ATOM | 186 | CG2 | THR | F | 68 | 51.539 | 114.564 | 181.073 | 1.00 | 44.19 | C |
| ATOM | 190 | C | THR | F | 68 | 49.213 | 112.945 | 182.002 | 1.00 | 39.50 | C |
| ATOM | 191 | O | THR | F | 68 | 49.642 | 113.420 | 183.065 | 1.00 | 37.57 | O |
| ATOM | 193 | N | ARG | F | 69 | 47.913 | 112.846 | 181.732 | 1.00 | 38.51 | N |
| ATOM | 194 | CA | ARG | F | 69 | 46.883 | 113.384 | 182.629 | 1.00 | 38.40 | C |
| ATOM | 196 | CB | ARG | F | 69 | 46.705 | 114.883 | 182.367 | 1.00 | 39.86 | C |
| ATOM | 199 | CG | ARG | F | 69 | 46.580 | 115.250 | 180.894 | 1.00 | 41.83 | C |
| ATOM | 202 | CD | ARG | F | 69 | 46.369 | 116.743 | 180.708 | 1.00 | 44.43 | C |
| ATOM | 205 | NE | ARG | F | 69 | 44.999 | 117.053 | 180.298 | 1.00 | 46.17 | N |
| ATOM | 207 | CZ | ARG | F | 69 | 44.596 | 117.244 | 179.042 | 1.00 | 45.88 | C |
| ATOM | 208 | NH1 | ARG | F | 69 | 45.445 | 117.181 | 178.019 | 1.00 | 46.31 | N |
| ATOM | 211 | NH2 | ARG | F | 69 | 43.321 | 117.517 | 178.808 | 1.00 | 46.73 | N |
| ATOM | 214 | C | ARG | F | 69 | 45.548 | 112.661 | 182.453 | 1.00 | 35.07 | C |
| ATOM | 215 | O | ARG | F | 69 | 45.406 | 111.813 | 181.569 | 1.00 | 35.38 | O |
| ATOM | 217 | N | CYS | F | 70 | 44.589 | 112.995 | 183.316 | 1.00 | 32.36 | N |
| ATOM | 218 | CA | CYS | F | 70 | 43.234 | 112.452 | 183.276 | 1.00 | 35.55 | C |
| ATOM | 220 | CB | CYS | F | 70 | 42.866 | 111.835 | 184.635 | 1.00 | 38.07 | C |
| ATOM | 223 | SG | CYS | F | 70 | 41.159 | 111.188 | 184.842 | 1.00 | 42.72 | S |
| ATOM | 225 | C | CYS | F | 70 | 42.290 | 113.591 | 182.929 | 1.00 | 37.28 | C |
| ATOM | 226 | O | CYS | F | 70 | 42.532 | 114.745 | 183.307 | 1.00 | 33.85 | O |
| ATOM | 228 | N | GLU | F | 71 | 41.221 | 113.264 | 182.211 | 1.00 | 38.37 | N |
| ATOM | 229 | CA | GLU | F | 71 | 40.231 | 114.255 | 181.787 | 1.00 | 40.11 | C |
| ATOM | 231 | CB | GLU | F | 71 | 40.584 | 114.771 | 180.390 | 1.00 | 41.97 | C |
| ATOM | 234 | CG | GLU | F | 71 | 39.619 | 115.809 | 179.833 | 1.00 | 45.06 | C |
| ATOM | 237 | CD | GLU | F | 71 | 40.062 | 116.344 | 178.481 | 1.00 | 47.04 | C |
| ATOM | 238 | OE1 | GLU | F | 71 | 39.818 | 115.668 | 177.458 | 1.00 | 49.11 | O |
| ATOM | 239 | OE2 | GLU | F | 71 | 40.643 | 117.446 | 178.445 | 1.00 | 50.33 | O |
| ATOM | 240 | C | GLU | F | 71 | 38.854 | 113.610 | 181.766 | 1.00 | 37.40 | C |
| ATOM | 241 | O | GLU | F | 71 | 38.656 | 112.614 | 181.076 | 1.00 | 37.09 | O |
| ATOM | 243 | N | ASP | F | 72 | 37.916 | 114.171 | 182.524 | 1.00 | 36.72 | N |
| ATOM | 244 | CA | ASP | F | 72 | 36.539 | 113.671 | 182.579 | 1.00 | 40.16 | C |
| ATOM | 246 | CB | ASP | F | 72 | 35.800 | 113.970 | 181.261 | 1.00 | 45.47 | C |
| ATOM | 249 | CG | ASP | F | 72 | 35.573 | 115.457 | 181.036 | 1.00 | 48.82 | C |
| ATOM | 250 | OD1 | ASP | F | 72 | 34.968 | 116.111 | 181.913 | 1.00 | 50.23 | O |
| ATOM | 251 | OD2 | ASP | F | 72 | 35.984 | 115.969 | 179.969 | 1.00 | 51.56 | O |
| ATOM | 252 | C | ASP | F | 72 | 36.480 | 112.170 | 182.898 | 1.00 | 39.76 | C |
| ATOM | 253 | O | ASP | F | 72 | 35.778 | 111.404 | 182.229 | 1.00 | 37.08 | O |
| ATOM | 255 | N | GLY | F | 73 | 37.230 | 111.761 | 183.921 | 1.00 | 38.04 | N |
| ATOM | 256 | CA | GLY | F | 73 | 37.203 | 110.380 | 184.404 | 1.00 | 35.82 | C |
| ATOM | 259 | C | GLY | F | 73 | 37.906 | 109.374 | 183.518 | 1.00 | 30.45 | C |
| ATOM | 260 | O | GLY | F | 73 | 37.759 | 108.164 | 183.718 | 1.00 | 30.26 | O |
| ATOM | 262 | N | GLN | F | 74 | 38.681 | 109.858 | 182.549 | 1.00 | 32.17 | N |
| ATOM | 263 | CA | GLN | F | 74 | 39.390 | 108.997 | 181.607 | 1.00 | 35.21 | C |
| ATOM | 265 | CB | GLN | F | 74 | 38.754 | 109.061 | 180.216 | 1.00 | 38.25 | C |
| ATOM | 268 | CG | GLN | F | 74 | 37.283 | 108.714 | 180.192 | 1.00 | 39.40 | C |
| ATOM | 271 | CD | GLN | F | 74 | 36.739 | 108.444 | 178.795 | 1.00 | 39.13 | C |
| ATOM | 272 | OE1 | GLN | F | 74 | 35.541 | 108.211 | 178.634 | 1.00 | 42.87 | O |
| ATOM | 273 | NE2 | GLN | F | 74 | 37.607 | 108.468 | 177.786 | 1.00 | 35.70 | N |
| ATOM | 276 | C | GLN | F | 74 | 40.838 | 109.412 | 181.484 | 1.00 | 33.47 | C |
| ATOM | 277 | O | GLN | F | 74 | 41.155 | 110.599 | 181.560 | 1.00 | 37.31 | O |
| ATOM | 279 | N | LEU | F | 75 | 41.718 | 108.434 | 181.299 | 1.00 | 34.11 | N |
| ATOM | 280 | CA | LEU | F | 75 | 43.094 | 108.720 | 180.928 | 1.00 | 35.05 | C |
| ATOM | 282 | CB | LEU | F | 75 | 43.938 | 107.442 | 180.859 | 1.00 | 37.04 | C |
| ATOM | 285 | CG | LEU | F | 75 | 44.143 | 106.655 | 182.167 | 1.00 | 38.44 | C |
| ATOM | 287 | CD1 | LEU | F | 75 | 44.996 | 105.442 | 181.923 | 1.00 | 40.51 | C |
| ATOM | 291 | CD2 | LEU | F | 75 | 44.765 | 107.522 | 183.248 | 1.00 | 41.38 | C |
| ATOM | 295 | C | LEU | F | 75 | 43.051 | 109.428 | 179.576 | 1.00 | 34.62 | C |
| ATOM | 296 | O | LEU | F | 75 | 42.324 | 109.016 | 178.667 | 1.00 | 33.35 | O |
| ATOM | 298 | N | PHE | F | 76 | 43.802 | 110.517 | 179.452 | 1.00 | 36.91 | N |
| ATOM | 299 | CA | PHE | F | 76 | 43.769 | 111.305 | 178.226 | 1.00 | 35.73 | C |
| ATOM | 301 | CB | PHE | F | 76 | 43.981 | 112.795 | 178.510 | 1.00 | 38.02 | C |
| ATOM | 304 | CG | PHE | F | 76 | 43.891 | 113.648 | 177.284 | 1.00 | 36.39 | C |
| ATOM | 305 | CD1 | PHE | F | 76 | 42.654 | 113.991 | 176.756 | 1.00 | 39.22 | C |
| ATOM | 307 | CE1 | PHE | F | 76 | 42.562 | 114.766 | 175.601 | 1.00 | 39.33 | C |
| ATOM | 309 | CZ | PHE | F | 76 | 43.719 | 115.196 | 174.962 | 1.00 | 39.59 | C |
| ATOM | 311 | CE2 | PHE | F | 76 | 44.964 | 114.852 | 175.479 | 1.00 | 39.17 | C |
| ATOM | 313 | CD2 | PHE | F | 76 | 45.043 | 114.077 | 176.631 | 1.00 | 39.39 | C |
| ATOM | 315 | C | PHE | F | 76 | 44.835 | 110.788 | 177.266 | 1.00 | 32.41 | C |
| ATOM | 316 | O | PHE | F | 76 | 46.010 | 110.739 | 177.618 | 1.00 | 32.71 | O |
| ATOM | 318 | N | ILE | F | 77 | 44.417 | 110.422 | 176.055 | 1.00 | 32.77 | N |
| ATOM | 319 | CA | ILE | F | 77 | 45.323 | 109.882 | 175.035 | 1.00 | 33.31 | C |
| ATOM | 321 | CB | ILE | F | 77 | 44.658 | 108.709 | 174.256 | 1.00 | 33.72 | C |
| ATOM | 323 | CG1 | ILE | F | 77 | 44.113 | 107.648 | 175.227 | 1.00 | 33.14 | C |
| ATOM | 326 | CD1 | ILE | F | 77 | 43.069 | 106.717 | 174.615 | 1.00 | 32.80 | C |
| ATOM | 330 | CG2 | ILE | F | 77 | 45.655 | 108.072 | 173.262 | 1.00 | 34.01 | C |
| ATOM | 334 | C | ILE | F | 77 | 45.746 | 110.985 | 174.054 | 1.00 | 33.50 | C |
| ATOM | 335 | O | ILE | F | 77 | 44.920 | 111.490 | 173.297 | 1.00 | 35.38 | O |
| ATOM | 337 | N | SER | F | 78 | 47.023 | 111.375 | 174.075 | 1.00 | 34.73 | N |
| ATOM | 338 | CA | SER | F | 78 | 47.544 | 112.275 | 173.044 | 1.00 | 35.69 | C |
| ATOM | 340 | CB | SER | F | 78 | 48.375 | 113.427 | 173.625 | 1.00 | 40.55 | C |
| ATOM | 343 | OG | SER | F | 78 | 49.463 | 112.965 | 174.389 | 1.00 | 49.39 | O |
| ATOM | 345 | C | SER | F | 78 | 48.326 | 111.478 | 171.997 | 1.00 | 33.95 | C |
| ATOM | 346 | O | SER | F | 78 | 49.422 | 110.966 | 172.253 | 1.00 | 31.82 | O |
| ATOM | 348 | N | SER | F | 79 | 47.724 | 111.372 | 170.821 | 1.00 | 34.55 | N |
| ATOM | 349 | CA | SER | F | 79 | 48.281 | 110.608 | 169.730 | 1.00 | 34.48 | C |
| ATOM | 351 | CB | SER | F | 79 | 47.220 | 110.363 | 168.661 | 1.00 | 38.17 | C |
| ATOM | 354 | OG | SER | F | 79 | 47.759 | 109.625 | 167.576 | 1.00 | 43.21 | O |
| ATOM | 356 | C | SER | F | 79 | 49.444 | 111.374 | 169.128 | 1.00 | 30.73 | C |
| ATOM | 357 | O | SER | F | 79 | 49.337 | 112.561 | 168.854 | 1.00 | 29.63 | O |
| ATOM | 359 | N | TYR | F | 80 | 50.549 | 110.680 | 168.912 | 1.00 | 29.49 | N |
| ATOM | 360 | CA | TYR | F | 80 | 51.695 | 111.272 | 168.234 | 1.00 | 32.34 | C |
| ATOM | 362 | CB | TYR | F | 80 | 52.895 | 110.343 | 168.353 | 1.00 | 31.61 | C |
| ATOM | 365 | CG | TYR | F | 80 | 53.440 | 110.242 | 169.761 | 1.00 | 31.19 | C |
| ATOM | 366 | CD1 | TYR | F | 80 | 54.246 | 111.250 | 170.283 | 1.00 | 34.23 | C |
| ATOM | 368 | CE1 | TYR | F | 80 | 54.753 | 111.171 | 171.564 | 1.00 | 33.04 | C |
| ATOM | 370 | CZ | TYR | F | 80 | 54.469 | 110.074 | 172.347 | 1.00 | 33.50 | C |
| ATOM | 371 | OH | TYR | F | 80 | 54.998 | 110.011 | 173.618 | 1.00 | 34.38 | O |
| ATOM | 373 | CE2 | TYR | F | 80 | 53.672 | 109.047 | 171.854 | 1.00 | 32.19 | C |
| ATOM | 375 | CD2 | TYR | F | 80 | 53.163 | 109.139 | 170.566 | 1.00 | 31.69 | C |
| ATOM | 377 | C | TYR | F | 80 | 51.371 | 111.515 | 166.765 | 1.00 | 31.18 | C |
| ATOM | 378 | O | TYR | F | 80 | 50.632 | 110.749 | 166.153 | 1.00 | 36.84 | O |
| ATOM | 380 | N | LYS | F | 81 | 51.931 | 112.578 | 166.210 | 1.00 | 35.15 | N |
| ATOM | 381 | CA | LYS | F | 81 | 51.785 | 112.887 | 164.794 | 1.00 | 39.56 | C |
| ATOM | 383 | CB | LYS | F | 81 | 51.661 | 114.401 | 164.593 | 1.00 | 49.16 | C |
| ATOM | 386 | CG | LYS | F | 81 | 50.452 | 115.025 | 165.298 | 1.00 | 53.98 | C |
| ATOM | 389 | CD | LYS | F | 81 | 49.136 | 114.668 | 164.601 | 1.00 | 58.66 | C |
| ATOM | 392 | CE | LYS | F | 81 | 47.915 | 115.125 | 165.402 | 1.00 | 58.13 | C |
| ATOM | 395 | NZ | LYS | F | 81 | 47.548 | 114.154 | 166.480 | 1.00 | 58.85 | N |
| ATOM | 399 | C | LYS | F | 81 | 53.007 | 112.338 | 164.070 | 1.00 | 36.05 | C |
| ATOM | 400 | O | LYS | F | 81 | 54.114 | 112.857 | 164.219 | 1.00 | 39.95 | O |
| ATOM | 402 | N | ASN | F | 82 | 52.802 | 111.266 | 163.312 | 1.00 | 32.29 | N |
| ATOM | 403 | CA | ASN | F | 82 | 53.898 | 110.538 | 162.692 | 1.00 | 30.77 | C |
| ATOM | 405 | CB | ASN | F | 82 | 54.628 | 109.685 | 163.768 | 1.00 | 36.31 | C |
| ATOM | 408 | CG | ASN | F | 82 | 55.861 | 108.954 | 163.225 | 1.00 | 38.97 | C |
| ATOM | 409 | OD1 | ASN | F | 82 | 55.764 | 107.801 | 162.828 | 1.00 | 42.48 | O |
| ATOM | 410 | ND2 | ASN | F | 82 | 57.022 | 109.627 | 163.210 | 1.00 | 36.16 | N |
| ATOM | 413 | C | ASN | F | 82 | 53.342 | 109.661 | 161.558 | 1.00 | 28.91 | C |
| ATOM | 414 | O | ASN | F | 82 | 52.192 | 109.183 | 161.620 | 1.00 | 27.08 | O |
| ATOM | 416 | N | GLU | F | 83 | 54.164 | 109.461 | 160.531 | 1.00 | 25.96 | N |
| ATOM | 417 | CA | GLU | F | 83 | 53.823 | 108.613 | 159.380 | 1.00 | 26.59 | C |
| ATOM | 419 | CB | GLU | F | 83 | 55.005 | 108.550 | 158.405 | 1.00 | 24.88 | C |
| ATOM | 422 | CG | GLU | F | 83 | 56.242 | 107.825 | 158.955 | 1.00 | 25.74 | C |
| ATOM | 425 | CD | GLU | F | 83 | 57.423 | 107.866 | 157.998 | 1.00 | 27.29 | C |
| ATOM | 426 | OE1 | GLU | F | 83 | 57.195 | 107.921 | 156.773 | 1.00 | 28.83 | O |
| ATOM | 427 | OE2 | GLU | F | 83 | 58.581 | 107.869 | 158.472 | 1.00 | 26.92 | O |
| ATOM | 428 | C | GLU | F | 83 | 53.411 | 107.184 | 159.735 | 1.00 | 29.86 | C |
| ATOM | 429 | O | GLU | F | 83 | 52.700 | 106.535 | 158.971 | 1.00 | 29.60 | O |
| ATOM | 431 | N | TYR | F | 84 | 53.870 | 106.678 | 160.874 | 1.00 | 26.37 | N |
| ATOM | 432 | CA | TYR | F | 84 | 53.591 | 105.303 | 161.230 | 1.00 | 28.42 | C |
| ATOM | 434 | CB | TYR | F | 84 | 54.711 | 104.749 | 162.118 | 1.00 | 29.04 | C |
| ATOM | 437 | CG | TYR | F | 84 | 56.069 | 104.653 | 161.436 | 1.00 | 24.31 | C |
| ATOM | 438 | CD1 | TYR | F | 84 | 56.197 | 104.177 | 160.134 | 1.00 | 25.74 | C |
| ATOM | 440 | CE1 | TYR | F | 84 | 57.446 | 104.071 | 159.524 | 1.00 | 26.82 | C |
| ATOM | 442 | CZ | TYR | F | 84 | 58.580 | 104.413 | 160.232 | 1.00 | 26.85 | C |
| ATOM | 443 | OH | TYR | F | 84 | 59.828 | 104.323 | 159.646 | 1.00 | 24.57 | O |
| ATOM | 445 | CE2 | TYR | F | 84 | 58.473 | 104.878 | 161.527 | 1.00 | 27.66 | C |
| ATOM | 447 | CD2 | TYR | F | 84 | 57.231 | 104.992 | 162.120 | 1.00 | 30.04 | C |
| ATOM | 449 | C | TYR | F | 84 | 52.227 | 105.102 | 161.891 | 1.00 | 29.64 | C |
| ATOM | 450 | O | TYR | F | 84 | 51.778 | 103.971 | 162.041 | 1.00 | 32.17 | O |
| ATOM | 452 | N | GLN | F | 85 | 51.570 | 106.193 | 162.280 | 1.00 | 26.50 | N |
| ATOM | 453 | CA | GLN | F | 85 | 50.234 | 106.112 | 162.875 | 1.00 | 27.04 | C |
| ATOM | 455 | CB | GLN | F | 85 | 49.885 | 107.436 | 163.564 | 1.00 | 32.00 | C |
| ATOM | 458 | CG | GLN | F | 85 | 50.882 | 107.869 | 164.648 | 1.00 | 32.59 | C |
| ATOM | 461 | CD | GLN | F | 85 | 50.553 | 107.299 | 166.025 | 1.00 | 35.31 | C |
| ATOM | 462 | OE1 | GLN | F | 85 | 50.402 | 106.098 | 166.188 | 1.00 | 32.90 | O |
| ATOM | 463 | NE2 | GLN | F | 85 | 50.443 | 108.170 | 167.016 | 1.00 | 39.36 | N |
| ATOM | 466 | C | GLN | F | 85 | 49.220 | 105.799 | 161.776 | 1.00 | 27.70 | C |
| ATOM | 467 | O | GLN | F | 85 | 49.162 | 106.499 | 160.765 | 1.00 | 28.29 | O |
| ATOM | 469 | N | THR | F | 86 | 48.443 | 104.734 | 161.943 | 1.00 | 30.20 | N |
| ATOM | 470 | CA | THR | F | 86 | 47.437 | 104.359 | 160.942 | 1.00 | 29.63 | C |
| ATOM | 472 | CB | THR | F | 86 | 47.811 | 103.038 | 160.214 | 1.00 | 29.27 | C |
| ATOM | 474 | OG1 | THR | F | 86 | 47.926 | 101.966 | 161.161 | 1.00 | 31.11 | O |
| ATOM | 476 | CG2 | THR | F | 86 | 49.117 | 103.189 | 159.458 | 1.00 | 30.95 | C |
| ATOM | 480 | C | THR | F | 86 | 46.007 | 104.237 | 161.507 | 1.00 | 30.59 | C |
| ATOM | 481 | O | THR | F | 86 | 45.116 | 103.764 | 160.809 | 1.00 | 30.88 | O |
| ATOM | 483 | N | MET | F | 87 | 45.799 | 104.650 | 162.758 | 1.00 | 32.49 | N |
| ATOM | 484 | CA | MET | F | 87 | 44.466 | 104.708 | 163.363 | 1.00 | 33.04 | C |
| ATOM | 486 | CB | MET | F | 87 | 44.309 | 103.665 | 164.469 | 1.00 | 32.57 | C |
| ATOM | 489 | CG | MET | F | 87 | 44.247 | 102.227 | 163.993 | 1.00 | 36.60 | C |
| ATOM | 492 | SD | MET | F | 87 | 43.834 | 101.052 | 165.330 | 1.00 | 43.06 | S |
| ATOM | 493 | CE | MET | F | 87 | 45.373 | 100.909 | 166.015 | 1.00 | 30.49 | C |
| ATOM | 497 | C | MET | F | 87 | 44.239 | 106.095 | 163.944 | 1.00 | 32.91 | C |
| ATOM | 498 | O | MET | F | 87 | 45.198 | 106.818 | 164.209 | 1.00 | 28.95 | O |
| ATOM | 500 | N | GLU | F | 88 | 42.969 | 106.451 | 164.144 | 1.00 | 32.67 | N |
| ATOM | 501 | CA | GLU | F | 88 | 42.588 | 107.750 | 164.707 | 1.00 | 32.31 | C |
| ATOM | 503 | CB | GLU | F | 88 | 41.406 | 108.365 | 163.937 | 1.00 | 39.84 | C |
| ATOM | 506 | CG | GLU | F | 88 | 41.591 | 108.523 | 162.430 | 1.00 | 48.31 | C |
| ATOM | 509 | CD | GLU | F | 88 | 42.645 | 109.550 | 162.040 | 1.00 | 55.04 | C |
| ATOM | 510 | OE1 | GLU | F | 88 | 42.588 | 110.699 | 162.529 | 1.00 | 57.19 | O |
| ATOM | 511 | OE2 | GLU | F | 88 | 43.527 | 109.206 | 161.218 | 1.00 | 60.26 | O |
| ATOM | 512 | C | GLU | F | 88 | 42.176 | 107.640 | 166.170 | 1.00 | 31.08 | C |
| ATOM | 513 | O | GLU | F | 88 | 41.656 | 106.606 | 166.616 | 1.00 | 29.96 | O |
| ATOM | 515 | N | VAL | F | 89 | 42.431 | 108.719 | 166.910 | 1.00 | 29.46 | N |
| ATOM | 516 | CA | VAL | F | 89 | 41.854 | 108.942 | 168.225 | 1.00 | 33.88 | C |
| ATOM | 518 | CB | VAL | F | 89 | 42.908 | 109.452 | 169.232 | 1.00 | 34.18 | C |
| ATOM | 520 | CG1 | VAL | F | 89 | 42.245 | 109.950 | 170.527 | 1.00 | 35.93 | C |
| ATOM | 524 | CG2 | VAL | F | 89 | 43.905 | 108.362 | 169.535 | 1.00 | 34.05 | C |
| ATOM | 528 | C | VAL | F | 89 | 40.744 | 109.978 | 168.063 | 1.00 | 34.40 | C |
| ATOM | 529 | O | VAL | F | 89 | 40.971 | 111.034 | 167.495 | 1.00 | 34.04 | O |
| ATOM | 531 | N | GLN | F | 90 | 39.546 | 109.658 | 168.545 | 1.00 | 33.43 | N |
| ATOM | 532 | CA | GLN | F | 90 | 38.406 | 110.577 | 168.508 | 1.00 | 36.87 | C |
| ATOM | 534 | CB | GLN | F | 90 | 37.469 | 110.213 | 167.354 | 1.00 | 40.50 | C |
| ATOM | 537 | CG | GLN | F | 90 | 38.030 | 110.550 | 165.973 | 1.00 | 47.27 | C |
| ATOM | 540 | CD | GLN | F | 90 | 37.178 | 110.007 | 164.833 | 1.00 | 49.85 | C |
| ATOM | 541 | OE1 | GLN | F | 90 | 35.946 | 110.033 | 164.898 | 1.00 | 56.89 | O |
| ATOM | 542 | NE2 | GLN | F | 90 | 37.835 | 109.510 | 163.780 | 1.00 | 53.34 | N |
| ATOM | 545 | C | GLN | F | 90 | 37.665 | 110.502 | 169.839 | 1.00 | 33.05 | C |
| ATOM | 546 | O | GLN | F | 90 | 37.355 | 109.412 | 170.313 | 1.00 | 33.30 | O |
| ATOM | 548 | N | ASN | F | 91 | 37.400 | 111.654 | 170.445 | 1.00 | 30.67 | N |
| ATOM | 549 | CA | ASN | F | 91 | 36.770 | 111.725 | 171.772 | 1.00 | 34.21 | C |
| ATOM | 551 | CB | ASN | F | 91 | 35.313 | 111.282 | 171.699 | 1.00 | 37.29 | C |
| ATOM | 554 | CG | ASN | F | 91 | 34.498 | 112.139 | 170.763 | 1.00 | 40.36 | C |
| ATOM | 555 | OD1 | ASN | F | 91 | 34.723 | 113.343 | 170.656 | 1.00 | 41.82 | O |
| ATOM | 556 | ND2 | ASN | F | 91 | 33.549 | 111.524 | 170.076 | 1.00 | 44.04 | N |
| ATOM | 559 | C | ASN | F | 91 | 37.511 | 110.904 | 172.825 | 1.00 | 31.42 | C |
| ATOM | 560 | O | ASN | F | 91 | 36.896 | 110.223 | 173.650 | 1.00 | 28.99 | O |
| ATOM | 562 | N | ASN | F | 92 | 38.836 | 110.980 | 172.772 | 1.00 | 31.15 | N |
| ATOM | 563 | CA | ASN | F | 92 | 39.710 | 110.303 | 173.723 | 1.00 | 32.78 | C |
| ATOM | 565 | CB | ASN | F | 92 | 39.471 | 110.839 | 175.139 | 1.00 | 35.47 | C |
| ATOM | 568 | CG | ASN | F | 92 | 40.611 | 110.516 | 176.079 | 1.00 | 36.48 | C |
| ATOM | 569 | OD1 | ASN | F | 92 | 41.754 | 110.399 | 175.650 | 1.00 | 38.20 | O |
| ATOM | 570 | ND2 | ASN | F | 92 | 40.301 | 110.342 | 177.363 | 1.00 | 34.17 | N |
| ATOM | 573 | C | ASN | F | 92 | 39.591 | 108.772 | 173.687 | 1.00 | 33.20 | C |
| ATOM | 574 | O | ASN | F | 92 | 39.771 | 108.098 | 174.703 | 1.00 | 35.43 | O |
| ATOM | 576 | N | SER | F | 93 | 39.309 | 108.233 | 172.504 | 1.00 | 32.41 | N |
| ATOM | 577 | CA | SER | F | 93 | 39.234 | 106.787 | 172.298 | 1.00 | 31.44 | C |
| ATOM | 579 | CB | SER | F | 93 | 37.777 | 106.303 | 172.314 | 1.00 | 34.24 | C |
| ATOM | 582 | OG | SER | F | 93 | 37.075 | 106.750 | 171.172 | 1.00 | 35.99 | O |
| ATOM | 584 | C | SER | F | 93 | 39.905 | 106.446 | 170.972 | 1.00 | 30.94 | C |
| ATOM | 585 | O | SER | F | 93 | 39.880 | 107.242 | 170.040 | 1.00 | 28.84 | O |
| ATOM | 587 | N | VAL | F | 94 | 40.534 | 105.276 | 170.907 | 1.00 | 31.26 | N |
| ATOM | 588 | CA | VAL | F | 94 | 41.138 | 104.796 | 169.675 | 1.00 | 30.70 | C |
| ATOM | 590 | CB | VAL | F | 94 | 42.272 | 103.790 | 169.946 | 1.00 | 30.85 | C |
| ATOM | 592 | CG1 | VAL | F | 94 | 42.922 | 103.349 | 168.633 | 1.00 | 32.42 | C |
| ATOM | 596 | CG2 | VAL | F | 94 | 43.315 | 104.389 | 170.887 | 1.00 | 29.31 | C |
| ATOM | 600 | C | VAL | F | 94 | 40.033 | 104.128 | 168.846 | 1.00 | 32.37 | C |
| ATOM | 601 | O | VAL | F | 94 | 39.396 | 103.187 | 169.314 | 1.00 | 31.63 | O |
| ATOM | 603 | N | VAL | F | 95 | 39.827 | 104.617 | 167.624 | 1.00 | 31.16 | N |
| ATOM | 604 | CA | VAL | F | 95 | 38.732 | 104.160 | 166.766 | 1.00 | 30.98 | C |
| ATOM | 606 | CB | VAL | F | 95 | 38.279 | 105.268 | 165.789 | 1.00 | 32.06 | C |
| ATOM | 608 | CG1 | VAL | F | 95 | 37.092 | 104.800 | 164.940 | 1.00 | 33.64 | C |
| ATOM | 612 | CG2 | VAL | F | 95 | 37.906 | 106.540 | 166.552 | 1.00 | 32.86 | C |
| ATOM | 616 | C | VAL | F | 95 | 39.147 | 102.905 | 165.995 | 1.00 | 31.81 | C |
| ATOM | 617 | O | VAL | F | 95 | 40.138 | 102.906 | 165.253 | 1.00 | 30.06 | O |
| ATOM | 619 | N | ILE | F | 96 | 38.393 | 101.827 | 166.178 | 1.00 | 29.04 | N |
| ATOM | 620 | CA | ILE | F | 96 | 38.710 | 100.575 | 165.507 | 1.00 | 32.14 | C |
| ATOM | 622 | CB | ILE | F | 96 | 38.375 | 99.330 | 166.364 | 1.00 | 29.02 | C |
| ATOM | 624 | CG1 | ILE | F | 96 | 39.087 | 99.389 | 167.722 | 1.00 | 31.90 | C |
| ATOM | 627 | CD1 | ILE | F | 96 | 40.635 | 99.508 | 167.644 | 1.00 | 31.87 | C |
| ATOM | 631 | CG2 | ILE | F | 96 | 38.748 | 98.039 | 165.626 | 1.00 | 29.24 | C |
| ATOM | 635 | C | ILE | F | 96 | 37.951 | 100.556 | 164.187 | 1.00 | 30.25 | C |
| ATOM | 636 | O | ILE | F | 96 | 36.724 | 100.489 | 164.165 | 1.00 | 31.38 | O |
| ATOM | 638 | N | LYS | F | 97 | 38.715 | 100.642 | 163.099 | 1.00 | 30.81 | N |
| ATOM | 639 | CA | LYS | F | 97 | 38.206 | 100.605 | 161.738 | 1.00 | 33.46 | C |
| ATOM | 641 | CB | LYS | F | 97 | 39.077 | 101.510 | 160.850 | 1.00 | 34.21 | C |
| ATOM | 644 | CG | LYS | F | 97 | 38.629 | 101.605 | 159.407 | 1.00 | 37.93 | C |
| ATOM | 647 | CD | LYS | F | 97 | 39.396 | 102.694 | 158.635 | 1.00 | 40.98 | C |
| ATOM | 650 | CE | LYS | F | 97 | 40.757 | 102.204 | 158.140 | 1.00 | 41.89 | C |
| ATOM | 653 | NZ | LYS | F | 97 | 41.608 | 103.331 | 157.631 | 1.00 | 42.34 | N |
| ATOM | 657 | C | LYS | F | 97 | 38.218 | 99.184 | 161.173 | 1.00 | 30.79 | C |
| ATOM | 658 | O | LYS | F | 97 | 37.384 | 98.847 | 160.344 | 1.00 | 29.59 | O |
| ATOM | 660 | N | CYS | F | 98 | 39.188 | 98.375 | 161.594 | 1.00 | 31.57 | N |
| ATOM | 661 | CA | CYS | F | 98 | 39.398 | 97.044 | 161.033 | 1.00 | 29.43 | C |
| ATOM | 663 | CB | CYS | F | 98 | 40.778 | 96.936 | 160.384 | 1.00 | 32.50 | C |
| ATOM | 666 | SG | CYS | F | 98 | 41.146 | 98.189 | 159.146 | 1.00 | 33.96 | S |
| ATOM | 668 | C | CYS | F | 98 | 39.278 | 95.943 | 162.064 | 1.00 | 32.27 | C |
| ATOM | 669 | O | CYS | F | 98 | 39.805 | 96.064 | 163.176 | 1.00 | 33.15 | O |
| ATOM | 671 | N | ASP | F | 99 | 38.615 | 94.854 | 161.678 | 1.00 | 30.84 | N |
| ATOM | 672 | CA | ASP | F | 99 | 38.546 | 93.642 | 162.503 | 1.00 | 27.70 | C |
| ATOM | 674 | CB | ASP | F | 99 | 37.695 | 92.547 | 161.845 | 1.00 | 29.90 | C |
| ATOM | 677 | CG | ASP | F | 99 | 36.210 | 92.837 | 161.832 | 1.00 | 32.65 | C |
| ATOM | 678 | OD1 | ASP | F | 99 | 35.751 | 93.874 | 162.350 | 1.00 | 29.49 | O |
| ATOM | 679 | OD2 | ASP | F | 99 | 35.481 | 91.980 | 161.284 | 1.00 | 29.45 | O |
| ATOM | 680 | C | ASP | F | 99 | 39.949 | 93.074 | 162.631 | 1.00 | 30.45 | C |
| ATOM | 681 | O | ASP | F | 99 | 40.766 | 93.213 | 161.715 | 1.00 | 29.85 | O |
| ATOM | 683 | N | GLY | F | 100 | 40.210 | 92.408 | 163.753 | 1.00 | 32.83 | N |
| ATOM | 684 | CA | GLY | F | 100 | 41.456 | 91.677 | 163.946 | 1.00 | 31.74 | C |
| ATOM | 687 | C | GLY | F | 100 | 41.712 | 91.334 | 165.399 | 1.00 | 30.54 | C |
| ATOM | 688 | O | GLY | F | 100 | 40.989 | 91.784 | 166.288 | 1.00 | 28.92 | O |
| ATOM | 690 | N | LEU | F | 101 | 42.729 | 90.512 | 165.631 | 1.00 | 29.77 | N |
| ATOM | 691 | CA | LEU | F | 101 | 43.342 | 90.401 | 166.958 | 1.00 | 29.75 | C |
| ATOM | 693 | CB | LEU | F | 101 | 44.106 | 89.091 | 167.122 | 1.00 | 31.87 | C |
| ATOM | 696 | CG | LEU | F | 101 | 43.290 | 87.796 | 167.046 | 1.00 | 32.91 | C |
| ATOM | 698 | CD1 | LEU | F | 101 | 44.196 | 86.596 | 167.260 | 1.00 | 33.15 | C |
| ATOM | 702 | CD2 | LEU | F | 101 | 42.166 | 87.823 | 168.085 | 1.00 | 34.94 | C |
| ATOM | 706 | C | LEU | F | 101 | 44.303 | 91.570 | 167.107 | 1.00 | 29.86 | C |
| ATOM | 707 | O | LEU | F | 101 | 45.078 | 91.869 | 166.183 | 1.00 | 29.98 | O |
| ATOM | 709 | N | TYR | F | 102 | 44.224 | 92.235 | 168.253 | 1.00 | 29.08 | N |
| ATOM | 710 | CA | TYR | F | 102 | 45.084 | 93.361 | 168.573 | 1.00 | 29.61 | C |
| ATOM | 712 | CB | TYR | F | 102 | 44.263 | 94.643 | 168.701 | 1.00 | 29.60 | C |
| ATOM | 715 | CG | TYR | F | 102 | 43.726 | 95.259 | 167.423 | 1.00 | 27.77 | C |
| ATOM | 716 | CD1 | TYR | F | 102 | 42.613 | 94.731 | 166.779 | 1.00 | 26.54 | C |
| ATOM | 718 | CE1 | TYR | F | 102 | 42.087 | 95.319 | 165.628 | 1.00 | 27.73 | C |
| ATOM | 720 | CZ | TYR | F | 102 | 42.662 | 96.461 | 165.121 | 1.00 | 30.91 | C |
| ATOM | 721 | OH | TYR | F | 102 | 42.149 | 97.073 | 163.988 | 1.00 | 29.61 | O |
| ATOM | 723 | CE2 | TYR | F | 102 | 43.759 | 97.023 | 165.758 | 1.00 | 29.67 | C |
| ATOM | 725 | CD2 | TYR | F | 102 | 44.280 | 96.422 | 166.906 | 1.00 | 29.10 | C |
| ATOM | 727 | C | TYR | F | 102 | 45.767 | 93.125 | 169.908 | 1.00 | 29.19 | C |
| ATOM | 728 | O | TYR | F | 102 | 45.204 | 92.495 | 170.812 | 1.00 | 29.11 | O |
| ATOM | 730 | N | ILE | F | 103 | 46.976 | 93.655 | 170.034 | 1.00 | 27.81 | N |
| ATOM | 731 | CA | ILE | F | 103 | 47.587 | 93.858 | 171.329 | 1.00 | 30.85 | C |
| ATOM | 733 | CB | ILE | F | 103 | 49.120 | 93.760 | 171.301 | 1.00 | 33.46 | C |
| ATOM | 735 | CG1 | ILE | F | 103 | 49.558 | 92.399 | 170.829 | 1.00 | 37.09 | C |
| ATOM | 738 | CD1 | ILE | F | 103 | 51.082 | 92.262 | 170.725 | 1.00 | 38.79 | C |
| ATOM | 742 | CG2 | ILE | F | 103 | 49.718 | 94.038 | 172.702 | 1.00 | 36.79 | C |
| ATOM | 746 | C | ILE | F | 103 | 47.233 | 95.257 | 171.784 | 1.00 | 31.01 | C |
| ATOM | 747 | O | ILE | F | 103 | 47.336 | 96.221 | 171.014 | 1.00 | 31.53 | O |
| ATOM | 749 | N | ILE | F | 104 | 46.819 | 95.362 | 173.039 | 1.00 | 30.88 | N |
| ATOM | 750 | CA | ILE | F | 104 | 46.702 | 96.650 | 173.717 | 1.00 | 31.77 | C |
| ATOM | 752 | CB | ILE | F | 104 | 45.270 | 96.909 | 174.191 | 1.00 | 34.45 | C |
| ATOM | 754 | CG1 | ILE | F | 104 | 44.320 | 96.848 | 172.991 | 1.00 | 34.38 | C |
| ATOM | 757 | CD1 | ILE | F | 104 | 42.917 | 96.635 | 173.363 | 1.00 | 40.10 | C |
| ATOM | 761 | CG2 | ILE | F | 104 | 45.156 | 98.271 | 174.871 | 1.00 | 35.31 | C |
| ATOM | 765 | C | ILE | F | 104 | 47.717 | 96.642 | 174.870 | 1.00 | 31.64 | C |
| ATOM | 766 | O | ILE | F | 104 | 47.730 | 95.726 | 175.718 | 1.00 | 30.59 | O |
| ATOM | 768 | N | TYR | F | 105 | 48.600 | 97.635 | 174.851 | 1.00 | 28.00 | N |
| ATOM | 769 | CA | TYR | F | 105 | 49.676 | 97.765 | 175.816 | 1.00 | 29.44 | C |
| ATOM | 771 | CB | TYR | F | 105 | 51.045 | 97.681 | 175.133 | 1.00 | 36.38 | C |
| ATOM | 774 | CG | TYR | F | 105 | 52.184 | 97.634 | 176.126 | 1.00 | 34.89 | C |
| ATOM | 775 | CD1 | TYR | F | 105 | 52.406 | 96.488 | 176.858 | 1.00 | 40.95 | C |
| ATOM | 777 | CE1 | TYR | F | 105 | 53.421 | 96.404 | 177.775 | 1.00 | 42.22 | C |
| ATOM | 779 | CZ | TYR | F | 105 | 54.239 | 97.471 | 177.987 | 1.00 | 39.23 | C |
| ATOM | 780 | OH | TYR | F | 105 | 55.245 | 97.309 | 178.921 | 1.00 | 44.74 | O |
| ATOM | 782 | CE2 | TYR | F | 105 | 54.067 | 98.647 | 177.277 | 1.00 | 38.05 | C |
| ATOM | 784 | CD2 | TYR | F | 105 | 53.023 | 98.730 | 176.345 | 1.00 | 41.78 | C |
| ATOM | 786 | C | TYR | F | 105 | 49.561 | 99.114 | 176.517 | 1.00 | 28.35 | C |
| ATOM | 787 | O | TYR | F | 105 | 49.432 | 100.142 | 175.857 | 1.00 | 29.78 | O |
| ATOM | 789 | N | LEU | F | 106 | 49.625 | 99.101 | 177.842 | 1.00 | 29.59 | N |
| ATOM | 790 | CA | LEU | F | 106 | 49.555 | 100.334 | 178.646 | 1.00 | 27.33 | C |
| ATOM | 792 | CB | LEU | F | 106 | 48.207 | 100.437 | 179.373 | 1.00 | 29.18 | C |
| ATOM | 795 | CG | LEU | F | 106 | 47.986 | 101.676 | 180.261 | 1.00 | 30.55 | C |
| ATOM | 797 | CD1 | LEU | F | 106 | 47.974 | 102.952 | 179.432 | 1.00 | 33.47 | C |
| ATOM | 801 | CD2 | LEU | F | 106 | 46.700 | 101.547 | 181.052 | 1.00 | 33.66 | C |
| ATOM | 805 | C | LEU | F | 106 | 50.671 | 100.328 | 179.663 | 1.00 | 24.41 | C |
| ATOM | 806 | O | LEU | F | 106 | 50.889 | 99.327 | 180.344 | 1.00 | 25.05 | O |
| ATOM | 808 | N | LYS | F | 107 | 51.384 | 101.446 | 179.765 | 1.00 | 28.15 | N |
| ATOM | 809 | CA | LYS | F | 107 | 52.314 | 101.645 | 180.852 | 1.00 | 30.42 | C |
| ATOM | 811 | CB | LYS | F | 107 | 53.757 | 101.428 | 180.390 | 1.00 | 36.27 | C |
| ATOM | 814 | CG | LYS | F | 107 | 54.255 | 102.445 | 179.396 | 1.00 | 41.94 | C |
| ATOM | 817 | CD | LYS | F | 107 | 55.678 | 102.135 | 178.947 | 1.00 | 42.96 | C |
| ATOM | 820 | CE | LYS | F | 107 | 56.695 | 102.527 | 179.974 | 1.00 | 46.90 | C |
| ATOM | 823 | NZ | LYS | F | 107 | 58.060 | 102.093 | 179.559 | 1.00 | 50.38 | N |
| ATOM | 827 | C | LYS | F | 107 | 52.135 | 103.043 | 181.411 | 1.00 | 28.87 | C |
| ATOM | 828 | O | LYS | F | 107 | 51.698 | 103.931 | 180.710 | 1.00 | 27.30 | O |
| ATOM | 830 | N | GLY | F | 108 | 52.452 | 103.209 | 182.686 | 1.00 | 30.50 | N |
| ATOM | 831 | CA | GLY | F | 108 | 52.399 | 104.511 | 183.331 | 1.00 | 29.77 | C |
| ATOM | 834 | C | GLY | F | 108 | 52.904 | 104.448 | 184.750 | 1.00 | 26.93 | C |
| ATOM | 835 | O | GLY | F | 108 | 52.855 | 103.393 | 185.388 | 1.00 | 30.37 | O |
| ATOM | 837 | N | SER | F | 109 | 53.399 | 105.581 | 185.240 | 1.00 | 26.48 | N |
| ATOM | 838 | CA | SER | F | 109 | 53.830 | 105.721 | 186.617 | 1.00 | 30.15 | C |
| ATOM | 840 | CB | SER | F | 109 | 55.329 | 106.041 | 186.690 | 1.00 | 33.60 | C |
| ATOM | 843 | OG | SER | F | 109 | 56.089 | 105.046 | 186.017 | 1.00 | 36.48 | O |
| ATOM | 845 | C | SER | F | 109 | 53.006 | 106.824 | 187.274 | 1.00 | 29.90 | C |
| ATOM | 846 | O | SER | F | 109 | 52.790 | 107.892 | 186.690 | 1.00 | 25.72 | O |
| ATOM | 848 | N | PHE | F | 110 | 52.545 | 106.542 | 188.485 | 1.00 | 28.54 | N |
| ATOM | 849 | CA | PHE | F | 110 | 51.640 | 107.405 | 189.217 | 1.00 | 28.31 | C |
| ATOM | 851 | CB | PHE | F | 110 | 50.295 | 106.693 | 189.397 | 1.00 | 31.72 | C |
| ATOM | 854 | CG | PHE | F | 110 | 49.648 | 106.280 | 188.094 | 1.00 | 29.50 | C |
| ATOM | 855 | CD1 | PHE | F | 110 | 50.055 | 105.131 | 187.434 | 1.00 | 33.96 | C |
| ATOM | 857 | CE1 | PHE | F | 110 | 49.475 | 104.754 | 186.234 | 1.00 | 32.61 | C |
| ATOM | 859 | CZ | PHE | F | 110 | 48.467 | 105.528 | 185.687 | 1.00 | 32.93 | C |
| ATOM | 861 | CE2 | PHE | F | 110 | 48.053 | 106.679 | 186.333 | 1.00 | 31.71 | C |
| ATOM | 863 | CD2 | PHE | F | 110 | 48.642 | 107.048 | 187.530 | 1.00 | 32.22 | C |
| ATOM | 865 | C | PHE | F | 110 | 52.284 | 107.709 | 190.560 | 1.00 | 30.21 | C |
| ATOM | 866 | O | PHE | F | 110 | 53.246 | 107.044 | 190.949 | 1.00 | 33.59 | O |
| ATOM | 868 | N | PHE | F | 111 | 51.774 | 108.725 | 191.249 | 1.00 | 28.68 | N |
| ATOM | 869 | CA | PHE | F | 111 | 52.260 | 109.095 | 192.582 | 1.00 | 28.57 | C |
| ATOM | 871 | CB | PHE | F | 111 | 52.461 | 110.607 | 192.691 | 1.00 | 28.27 | C |
| ATOM | 874 | CG | PHE | F | 111 | 53.390 | 111.181 | 191.653 | 1.00 | 28.09 | C |
| ATOM | 875 | CD1 | PHE | F | 111 | 54.613 | 110.580 | 191.376 | 1.00 | 28.65 | C |
| ATOM | 877 | CE1 | PHE | F | 111 | 55.472 | 111.118 | 190.427 | 1.00 | 27.93 | C |
| ATOM | 879 | CZ | PHE | F | 111 | 55.120 | 112.269 | 189.752 | 1.00 | 28.58 | C |
| ATOM | 881 | CE2 | PHE | F | 111 | 53.913 | 112.888 | 190.022 | 1.00 | 29.54 | C |
| ATOM | 883 | CD2 | PHE | F | 111 | 53.053 | 112.344 | 190.973 | 1.00 | 30.24 | C |
| ATOM | 885 | C | PHE | F | 111 | 51.290 | 108.630 | 193.672 | 1.00 | 28.64 | C |
| ATOM | 886 | O | PHE | F | 111 | 51.479 | 108.934 | 194.847 | 1.00 | 26.04 | O |
| ATOM | 888 | N | GLN | F | 112 | 50.258 | 107.896 | 193.273 | 1.00 | 30.69 | N |
| ATOM | 889 | CA | GLN | F | 112 | 49.271 | 107.359 | 194.204 | 1.00 | 34.46 | C |
| ATOM | 891 | CB | GLN | F | 112 | 48.232 | 108.425 | 194.557 | 1.00 | 35.32 | C |
| ATOM | 894 | CG | GLN | F | 112 | 47.366 | 108.852 | 193.384 | 1.00 | 37.97 | C |
| ATOM | 897 | CD | GLN | F | 112 | 46.567 | 110.093 | 193.671 | 1.00 | 39.33 | C |
| ATOM | 898 | OE1 | GLN | F | 112 | 45.601 | 110.058 | 194.434 | 1.00 | 44.84 | O |
| ATOM | 899 | NE2 | GLN | F | 112 | 46.962 | 111.209 | 193.060 | 1.00 | 41.29 | N |
| ATOM | 902 | C | GLN | F | 112 | 48.600 | 106.145 | 193.571 | 1.00 | 35.82 | C |
| ATOM | 903 | O | GLN | F | 112 | 48.712 | 105.925 | 192.357 | 1.00 | 31.47 | O |
| ATOM | 905 | N | GLU | F | 113 | 47.906 | 105.360 | 194.395 | 1.00 | 35.40 | N |
| ATOM | 906 | CA | GLU | F | 113 | 47.185 | 104.185 | 193.911 | 1.00 | 35.49 | C |
| ATOM | 908 | CB | GLU | F | 113 | 46.606 | 103.368 | 195.078 | 1.00 | 41.50 | C |
| ATOM | 911 | CG | GLU | F | 113 | 47.628 | 102.900 | 196.118 | 1.00 | 47.70 | C |
| ATOM | 914 | CD | GLU | F | 113 | 48.126 | 101.485 | 195.882 | 1.00 | 51.05 | C |
| ATOM | 915 | OE1 | GLU | F | 113 | 47.449 | 100.526 | 196.329 | 1.00 | 51.35 | O |
| ATOM | 916 | OE2 | GLU | F | 113 | 49.208 | 101.338 | 195.273 | 1.00 | 50.12 | O |
| ATOM | 917 | C | GLU | F | 113 | 46.048 | 104.646 | 193.009 | 1.00 | 29.04 | C |
| ATOM | 918 | O | GLU | F | 113 | 45.332 | 105.592 | 193.343 | 1.00 | 36.71 | O |
| ATOM | 920 | N | VAL | F | 114 | 45.890 | 103.979 | 191.876 | 1.00 | 35.77 | N |
| ATOM | 921 | CA | VAL | F | 114 | 44.790 | 104.246 | 190.960 | 1.00 | 34.63 | C |
| ATOM | 923 | CB | VAL | F | 114 | 45.247 | 105.048 | 189.699 | 1.00 | 31.03 | C |
| ATOM | 925 | CG1 | VAL | F | 114 | 45.879 | 106.377 | 190.118 | 1.00 | 35.12 | C |
| ATOM | 929 | CG2 | VAL | F | 114 | 46.202 | 104.241 | 188.843 | 1.00 | 32.85 | C |
| ATOM | 933 | C | VAL | F | 114 | 44.163 | 102.936 | 190.523 | 1.00 | 35.55 | C |
| ATOM | 934 | O | VAL | F | 114 | 44.835 | 101.901 | 190.474 | 1.00 | 36.33 | O |
| ATOM | 936 | N | LYS | F | 115 | 42.875 | 102.996 | 190.192 | 1.00 | 34.61 | N |
| ATOM | 937 | CA | LYS | F | 115 | 42.160 | 101.868 | 189.596 | 1.00 | 34.20 | C |
| ATOM | 939 | CB | LYS | F | 115 | 40.909 | 101.533 | 190.414 | 1.00 | 35.12 | C |
| ATOM | 942 | CG | LYS | F | 115 | 41.198 | 101.050 | 191.822 | 1.00 | 37.25 | C |
| ATOM | 945 | CD | LYS | F | 115 | 39.910 | 100.867 | 192.611 | 1.00 | 38.64 | C |
| ATOM | 948 | CE | LYS | F | 115 | 40.140 | 100.121 | 193.916 | 1.00 | 40.07 | C |
| ATOM | 951 | NZ | LYS | F | 115 | 38.844 | 99.810 | 194.597 | 1.00 | 41.65 | N |
| ATOM | 955 | C | LYS | F | 115 | 41.771 | 102.250 | 188.178 | 1.00 | 32.77 | C |
| ATOM | 956 | O | LYS | F | 115 | 40.984 | 103.173 | 187.977 | 1.00 | 33.31 | O |
| ATOM | 958 | N | ILE | F | 116 | 42.341 | 101.555 | 187.198 | 1.00 | 30.22 | N |
| ATOM | 959 | CA | ILE | F | 116 | 42.084 | 101.840 | 185.799 | 1.00 | 31.26 | C |
| ATOM | 961 | CB | ILE | F | 116 | 43.406 | 102.155 | 185.052 | 1.00 | 33.21 | C |
| ATOM | 963 | CG1 | ILE | F | 116 | 44.057 | 103.393 | 185.684 | 1.00 | 37.28 | C |
| ATOM | 966 | CD1 | ILE | F | 116 | 45.430 | 103.728 | 185.182 | 1.00 | 39.77 | C |
| ATOM | 970 | CG2 | ILE | F | 116 | 43.150 | 102.386 | 183.569 | 1.00 | 30.49 | C |
| ATOM | 974 | C | ILE | F | 116 | 41.324 | 100.679 | 185.151 | 1.00 | 30.44 | C |
| ATOM | 975 | O | ILE | F | 116 | 41.585 | 99.505 | 185.441 | 1.00 | 31.68 | O |
| ATOM | 977 | N | ASP | F | 117 | 40.356 | 101.029 | 184.310 | 1.00 | 29.80 | N |
| ATOM | 978 | CA | ASP | F | 117 | 39.560 | 100.061 | 183.556 | 1.00 | 29.58 | C |
| ATOM | 980 | CB | ASP | F | 117 | 38.069 | 100.173 | 183.900 | 1.00 | 32.71 | C |
| ATOM | 983 | CG | ASP | F | 117 | 37.729 | 99.675 | 185.301 | 1.00 | 37.22 | C |
| ATOM | 984 | OD1 | ASP | F | 117 | 38.305 | 98.672 | 185.762 | 1.00 | 35.20 | O |
| ATOM | 985 | OD2 | ASP | F | 117 | 36.844 | 100.288 | 185.946 | 1.00 | 40.61 | O |
| ATOM | 986 | C | ASP | F | 117 | 39.740 | 100.333 | 182.071 | 1.00 | 30.59 | C |
| ATOM | 987 | O | ASP | F | 117 | 39.736 | 101.488 | 181.638 | 1.00 | 29.98 | O |
| ATOM | 989 | N | LEU | F | 118 | 39.886 | 99.254 | 181.303 | 1.00 | 31.13 | N |
| ATOM | 990 | CA | LEU | F | 118 | 40.043 | 99.290 | 179.861 | 1.00 | 30.74 | C |
| ATOM | 992 | CB | LEU | F | 118 | 41.157 | 98.316 | 179.454 | 1.00 | 30.83 | C |
| ATOM | 995 | CG | LEU | F | 118 | 41.392 | 98.050 | 177.967 | 1.00 | 32.17 | C |
| ATOM | 997 | CD1 | LEU | F | 118 | 41.809 | 99.336 | 177.249 | 1.00 | 31.18 | C |
| ATOM | 1001 | CD2 | LEU | F | 118 | 42.452 | 96.974 | 177.802 | 1.00 | 34.09 | C |
| ATOM | 1005 | C | LEU | F | 118 | 38.734 | 98.868 | 179.194 | 1.00 | 31.68 | C |
| ATOM | 1006 | O | LEU | F | 118 | 38.199 | 97.798 | 179.487 | 1.00 | 29.27 | O |
| ATOM | 1008 | N | HIS | F | 119 | 38.244 | 99.710 | 178.288 | 1.00 | 31.23 | N |
| ATOM | 1009 | CA | HIS | F | 119 | 36.951 | 99.525 | 177.619 | 1.00 | 30.48 | C |
| ATOM | 1011 | CB | HIS | F | 119 | 36.047 | 100.749 | 177.815 | 1.00 | 29.71 | C |
| ATOM | 1014 | CG | HIS | F | 119 | 35.796 | 101.126 | 179.241 | 1.00 | 30.89 | C |
| ATOM | 1015 | ND1 | HIS | F | 119 | 34.597 | 100.879 | 179.875 | 1.00 | 31.76 | N |
| ATOM | 1017 | CE1 | HIS | F | 119 | 34.651 | 101.345 | 181.110 | 1.00 | 32.27 | C |
| ATOM | 1019 | NE2 | HIS | F | 119 | 35.833 | 101.906 | 181.294 | 1.00 | 31.34 | N |
| ATOM | 1021 | CD2 | HIS | F | 119 | 36.563 | 101.794 | 180.137 | 1.00 | 31.42 | C |
| ATOM | 1023 | C | HIS | F | 119 | 37.211 | 99.372 | 176.126 | 1.00 | 30.71 | C |
| ATOM | 1024 | O | HIS | F | 119 | 38.079 | 100.054 | 175.577 | 1.00 | 29.95 | O |
| ATOM | 1026 | N | PHE | F | 120 | 36.474 | 98.471 | 175.480 | 1.00 | 33.18 | N |
| ATOM | 1027 | CA | PHE | F | 120 | 36.547 | 98.265 | 174.028 | 1.00 | 33.86 | C |
| ATOM | 1029 | CB | PHE | F | 120 | 36.601 | 96.777 | 173.682 | 1.00 | 36.56 | C |
| ATOM | 1032 | CG | PHE | F | 120 | 37.809 | 96.053 | 174.230 | 1.00 | 36.09 | C |
| ATOM | 1033 | CD1 | PHE | F | 120 | 39.025 | 96.703 | 174.417 | 1.00 | 36.43 | C |
| ATOM | 1035 | CE1 | PHE | F | 120 | 40.120 | 96.017 | 174.912 | 1.00 | 39.05 | C |
| ATOM | 1037 | CZ | PHE | F | 120 | 40.025 | 94.671 | 175.199 | 1.00 | 38.81 | C |
| ATOM | 1039 | CE2 | PHE | F | 120 | 38.835 | 94.006 | 174.995 | 1.00 | 38.01 | C |
| ATOM | 1041 | CD2 | PHE | F | 120 | 37.736 | 94.698 | 174.507 | 1.00 | 38.78 | C |
| ATOM | 1043 | C | PHE | F | 120 | 35.354 | 98.870 | 173.300 | 1.00 | 34.66 | C |
| ATOM | 1044 | O | PHE | F | 120 | 35.360 | 98.970 | 172.077 | 1.00 | 31.76 | O |
| ATOM | 1046 | N | ARG | F | 121 | 34.327 | 99.250 | 174.049 | 1.00 | 35.83 | N |
| ATOM | 1047 | CA | ARG | F | 121 | 33.167 | 99.938 | 173.495 | 1.00 | 39.64 | C |
| ATOM | 1049 | CB | ARG | F | 121 | 32.336 | 99.014 | 172.589 | 1.00 | 39.98 | C |
| ATOM | 1052 | CG | ARG | F | 121 | 31.634 | 99.766 | 171.450 | 1.00 | 41.10 | C |
| ATOM | 1055 | CD | ARG | F | 121 | 31.195 | 98.855 | 170.317 | 1.00 | 44.27 | C |
| ATOM | 1058 | NE | ARG | F | 121 | 30.024 | 98.058 | 170.664 | 1.00 | 49.02 | N |
| ATOM | 1060 | CZ | ARG | F | 121 | 29.283 | 97.379 | 169.789 | 1.00 | 49.79 | C |
| ATOM | 1061 | NH1 | ARG | F | 121 | 29.563 | 97.399 | 168.487 | 1.00 | 49.55 | N |
| ATOM | 1064 | NH2 | ARG | F | 121 | 28.239 | 96.682 | 170.220 | 1.00 | 50.60 | N |
| ATOM | 1067 | C | ARG | F | 121 | 32.339 | 100.433 | 174.674 | 1.00 | 40.52 | C |
| ATOM | 1068 | O | ARG | F | 121 | 32.600 | 100.050 | 175.814 | 1.00 | 38.29 | O |
| ATOM | 1070 | N | GLU | F | 122 | 31.352 | 101.283 | 174.401 | 1.00 | 43.32 | N |
| ATOM | 1071 | CA | GLU | F | 122 | 30.535 | 101.893 | 175.459 | 1.00 | 45.49 | C |
| ATOM | 1073 | CB | GLU | F | 122 | 29.610 | 102.973 | 174.880 | 1.00 | 51.00 | C |
| ATOM | 1076 | CG | GLU | F | 122 | 30.324 | 104.264 | 174.458 | 1.00 | 55.20 | C |
| ATOM | 1079 | CD | GLU | F | 122 | 30.840 | 104.249 | 173.019 | 1.00 | 60.79 | C |
| ATOM | 1080 | OE1 | GLU | F | 122 | 31.196 | 103.164 | 172.491 | 1.00 | 61.68 | O |
| ATOM | 1081 | OE2 | GLU | F | 122 | 30.895 | 105.345 | 172.415 | 1.00 | 63.87 | O |
| ATOM | 1082 | C | GLU | F | 122 | 29.714 | 100.874 | 176.257 | 1.00 | 44.36 | C |
| ATOM | 1083 | O | GLU | F | 122 | 29.465 | 101.073 | 177.447 | 1.00 | 46.14 | O |
| ATOM | 1085 | N | ASP | F | 123 | 29.306 | 99.787 | 175.607 | 1.00 | 43.72 | N |
| ATOM | 1086 | CA | ASP | F | 123 | 28.521 | 98.731 | 176.266 | 1.00 | 46.27 | C |
| ATOM | 1088 | CB | ASP | F | 123 | 27.405 | 98.246 | 175.329 | 1.00 | 48.38 | C |
| ATOM | 1091 | CG | ASP | F | 123 | 27.930 | 97.760 | 173.984 | 1.00 | 49.94 | C |
| ATOM | 1092 | OD1 | ASP | F | 123 | 29.164 | 97.750 | 173.784 | 1.00 | 50.75 | O |
| ATOM | 1093 | OD2 | ASP | F | 123 | 27.102 | 97.387 | 173.126 | 1.00 | 50.56 | O |
| ATOM | 1094 | C | ASP | F | 123 | 29.362 | 97.533 | 176.752 | 1.00 | 47.24 | C |
| ATOM | 1095 | O | ASP | F | 123 | 28.820 | 96.575 | 177.300 | 1.00 | 49.69 | O |
| ATOM | 1097 | N | HIS | F | 124 | 30.678 | 97.597 | 176.545 | 1.00 | 46.31 | N |
| ATOM | 1098 | CA | HIS | F | 124 | 31.600 | 96.517 | 176.913 | 1.00 | 42.87 | C |
| ATOM | 1100 | CB | HIS | F | 124 | 32.914 | 96.716 | 176.133 | 1.00 | 44.77 | C |
| ATOM | 1103 | CG | HIS | F | 124 | 34.019 | 95.779 | 176.517 | 1.00 | 46.09 | C |
| ATOM | 1104 | ND1 | HIS | F | 124 | 35.171 | 96.210 | 177.139 | 1.00 | 44.42 | N |
| ATOM | 1106 | CE1 | HIS | F | 124 | 35.972 | 95.179 | 177.342 | 1.00 | 44.95 | C |
| ATOM | 1108 | NE2 | HIS | F | 124 | 35.379 | 94.094 | 176.878 | 1.00 | 43.97 | N |
| ATOM | 1110 | CD2 | HIS | F | 124 | 34.160 | 94.443 | 176.348 | 1.00 | 46.25 | C |
| ATOM | 1112 | C | HIS | F | 124 | 31.846 | 96.487 | 178.435 | 1.00 | 41.21 | C |
| ATOM | 1113 | O | HIS | F | 124 | 32.034 | 97.533 | 179.057 | 1.00 | 40.22 | O |
| ATOM | 1115 | N | ASN | F | 125 | 31.818 | 95.291 | 179.027 | 1.00 | 39.98 | N |
| ATOM | 1116 | CA | ASN | F | 125 | 32.217 | 95.091 | 180.430 | 1.00 | 39.41 | C |
| ATOM | 1118 | CB | ASN | F | 125 | 31.788 | 93.702 | 180.912 | 1.00 | 40.85 | C |
| ATOM | 1121 | CG | ASN | F | 125 | 31.936 | 93.513 | 182.423 | 1.00 | 43.60 | C |
| ATOM | 1122 | OD1 | ASN | F | 125 | 32.229 | 94.452 | 183.169 | 1.00 | 45.85 | O |
| ATOM | 1123 | ND2 | ASN | F | 125 | 31.713 | 92.286 | 182.879 | 1.00 | 44.49 | N |
| ATOM | 1126 | C | ASN | F | 125 | 33.741 | 95.244 | 180.534 | 1.00 | 38.97 | C |
| ATOM | 1127 | O | ASN | F | 125 | 34.479 | 94.435 | 179.965 | 1.00 | 37.29 | O |
| ATOM | 1129 | N | PRO | F | 126 | 34.221 | 96.280 | 181.248 | 1.00 | 38.29 | N |
| ATOM | 1130 | CA | PRO | F | 126 | 35.647 | 96.612 | 181.126 | 1.00 | 37.18 | C |
| ATOM | 1132 | CB | PRO | F | 126 | 35.741 | 98.012 | 181.744 | 1.00 | 38.83 | C |
| ATOM | 1135 | CG | PRO | F | 126 | 34.566 | 98.164 | 182.591 | 1.00 | 38.46 | C |
| ATOM | 1138 | CD | PRO | F | 126 | 33.517 | 97.186 | 182.170 | 1.00 | 38.80 | C |
| ATOM | 1141 | C | PRO | F | 126 | 36.608 | 95.660 | 181.830 | 1.00 | 36.04 | C |
| ATOM | 1142 | O | PRO | F | 126 | 36.219 | 94.941 | 182.748 | 1.00 | 35.04 | O |
| ATOM | 1143 | N | ILE | F | 127 | 37.864 | 95.684 | 181.390 | 1.00 | 33.47 | N |
| ATOM | 1144 | CA | ILE | F | 127 | 38.932 | 94.930 | 182.029 | 1.00 | 33.95 | C |
| ATOM | 1146 | CB | ILE | F | 127 | 40.004 | 94.497 | 181.000 | 1.00 | 33.78 | C |
| ATOM | 1148 | CG1 | ILE | F | 127 | 39.375 | 93.605 | 179.916 | 1.00 | 38.57 | C |
| ATOM | 1151 | CD1 | ILE | F | 127 | 40.238 | 93.459 | 178.650 | 1.00 | 41.00 | C |
| ATOM | 1155 | CG2 | ILE | F | 127 | 41.147 | 93.767 | 181.694 | 1.00 | 33.53 | C |
| ATOM | 1159 | C | ILE | F | 127 | 39.573 | 95.795 | 183.116 | 1.00 | 33.36 | C |
| ATOM | 1160 | O | ILE | F | 127 | 40.049 | 96.901 | 182.844 | 1.00 | 31.39 | O |
| ATOM | 1162 | N | SER | F | 128 | 39.576 | 95.286 | 184.342 | 1.00 | 27.99 | N |
| ATOM | 1163 | CA | SER | F | 128 | 40.252 | 95.947 | 185.460 | 1.00 | 33.77 | C |
| ATOM | 1165 | CB | SER | F | 128 | 39.746 | 95.389 | 186.791 | 1.00 | 35.44 | C |
| ATOM | 1168 | OG | SER | F | 128 | 40.492 | 95.881 | 187.895 | 1.00 | 40.27 | O |
| ATOM | 1170 | C | SER | F | 128 | 41.756 | 95.728 | 185.315 | 1.00 | 33.81 | C |
| ATOM | 1171 | O | SER | F | 128 | 42.216 | 94.593 | 185.300 | 1.00 | 31.64 | O |
| ATOM | 1173 | N | ILE | F | 129 | 42.516 | 96.809 | 185.201 | 1.00 | 31.18 | N |
| ATOM | 1174 | CA | ILE | F | 129 | 43.962 | 96.716 | 184.976 | 1.00 | 34.16 | C |
| ATOM | 1176 | CB | ILE | F | 129 | 44.484 | 97.959 | 184.202 | 1.00 | 32.56 | C |
| ATOM | 1178 | CG1 | ILE | F | 129 | 43.879 | 97.975 | 182.797 | 1.00 | 35.35 | C |
| ATOM | 1181 | CD1 | ILE | F | 129 | 44.259 | 99.180 | 181.970 | 1.00 | 39.13 | C |
| ATOM | 1185 | CG2 | ILE | F | 129 | 46.019 | 97.955 | 184.132 | 1.00 | 33.03 | C |
| ATOM | 1189 | C | ILE | F | 129 | 44.718 | 96.556 | 186.300 | 1.00 | 34.77 | C |
| ATOM | 1190 | O | ILE | F | 129 | 44.594 | 97.396 | 187.185 | 1.00 | 36.24 | O |
| ATOM | 1192 | N | PRO | F | 130 | 45.513 | 95.479 | 186.441 | 1.00 | 36.07 | N |
| ATOM | 1193 | CA | PRO | F | 130 | 46.278 | 95.334 | 187.679 | 1.00 | 38.75 | C |
| ATOM | 1195 | CB | PRO | F | 130 | 46.773 | 93.887 | 187.633 | 1.00 | 39.27 | C |
| ATOM | 1198 | CG | PRO | F | 130 | 46.688 | 93.468 | 186.221 | 1.00 | 40.09 | C |
| ATOM | 1201 | CD | PRO | F | 130 | 45.745 | 94.369 | 185.499 | 1.00 | 38.62 | C |
| ATOM | 1204 | C | PRO | F | 130 | 47.452 | 96.306 | 187.764 | 1.00 | 38.29 | C |
| ATOM | 1205 | O | PRO | F | 130 | 48.127 | 96.561 | 186.767 | 1.00 | 36.20 | O |
| ATOM | 1206 | N | MET | F | 131 | 47.667 | 96.856 | 188.952 | 1.00 | 39.61 | N |
| ATOM | 1207 | CA | MET | F | 131 | 48.851 | 97.652 | 189.239 | 1.00 | 44.26 | C |
| ATOM | 1209 | CB | MET | F | 131 | 48.554 | 98.694 | 190.322 | 1.00 | 47.71 | C |
| ATOM | 1212 | CG | MET | F | 131 | 48.597 | 100.106 | 189.813 | 1.00 | 49.46 | C |
| ATOM | 1215 | SD | MET | F | 131 | 48.545 | 101.349 | 191.097 | 1.00 | 48.37 | S |
| ATOM | 1216 | CE | MET | F | 131 | 49.320 | 102.681 | 190.193 | 1.00 | 49.64 | C |
| ATOM | 1220 | C | MET | F | 131 | 49.984 | 96.745 | 189.709 | 1.00 | 45.81 | C |
| ATOM | 1221 | O | MET | F | 131 | 49.734 | 95.736 | 190.369 | 1.00 | 43.21 | O |
| ATOM | 1223 | N | LEU | F | 132 | 51.222 | 97.116 | 189.375 | 1.00 | 48.02 | N |
| ATOM | 1224 | CA | LEU | F | 132 | 52.401 | 96.358 | 189.805 | 1.00 | 49.72 | C |
| ATOM | 1226 | CB | LEU | F | 132 | 53.685 | 96.889 | 189.159 | 1.00 | 50.32 | C |
| ATOM | 1229 | CG | LEU | F | 132 | 53.913 | 96.648 | 187.663 | 1.00 | 51.14 | C |
| ATOM | 1231 | CD1 | LEU | F | 132 | 55.320 | 97.066 | 187.295 | 1.00 | 51.30 | C |
| ATOM | 1235 | CD2 | LEU | F | 132 | 53.688 | 95.202 | 187.285 | 1.00 | 52.46 | C |
| ATOM | 1239 | C | LEU | F | 132 | 52.542 | 96.429 | 191.312 | 1.00 | 51.72 | C |
| ATOM | 1240 | O | LEU | F | 132 | 52.013 | 97.344 | 191.944 | 1.00 | 51.73 | O |
| ATOM | 1242 | N | ASN | F | 133 | 53.283 | 95.471 | 191.875 | 1.00 | 54.23 | N |
| ATOM | 1243 | CA | ASN | F | 133 | 53.499 | 95.377 | 193.324 | 1.00 | 53.52 | C |
| ATOM | 1245 | CB | ASN | F | 133 | 54.385 | 94.172 | 193.661 | 1.00 | 56.50 | C |
| ATOM | 1248 | CG | ASN | F | 133 | 53.841 | 92.869 | 193.094 | 1.00 | 57.08 | C |
| ATOM | 1249 | OD1 | ASN | F | 133 | 52.718 | 92.824 | 192.586 | 1.00 | 58.74 | O |
| ATOM | 1250 | ND2 | ASN | F | 133 | 54.640 | 91.809 | 193.166 | 1.00 | 55.18 | N |
| ATOM | 1253 | C | ASN | F | 133 | 54.121 | 96.653 | 193.880 | 1.00 | 54.30 | C |
| ATOM | 1254 | O | ASN | F | 133 | 54.017 | 96.942 | 195.075 | 1.00 | 53.43 | O |
| ATOM | 1256 | N | ASP | F | 134 | 54.794 | 97.379 | 192.989 | 1.00 | 54.15 | N |
| ATOM | 1257 | CA | ASP | F | 134 | 55.186 | 98.777 | 193.174 | 1.00 | 55.50 | C |
| ATOM | 1259 | CB | ASP | F | 134 | 55.470 | 99.394 | 191.784 | 1.00 | 56.66 | C |
| ATOM | 1262 | CG | ASP | F | 134 | 56.704 | 100.262 | 191.762 | 1.00 | 61.23 | C |
| ATOM | 1263 | OD1 | ASP | F | 134 | 57.701 | 99.916 | 192.433 | 1.00 | 66.25 | O |
| ATOM | 1264 | OD2 | ASP | F | 134 | 56.691 | 101.288 | 191.049 | 1.00 | 64.56 | O |
| ATOM | 1265 | C | ASP | F | 134 | 54.118 | 99.622 | 193.887 | 1.00 | 54.08 | C |
| ATOM | 1266 | O | ASP | F | 134 | 54.436 | 100.428 | 194.757 | 1.00 | 56.90 | O |
| ATOM | 1268 | N | GLY | F | 135 | 52.856 | 99.440 | 193.500 | 1.00 | 53.84 | N |
| ATOM | 1269 | CA | GLY | F | 135 | 51.767 | 100.315 | 193.937 | 1.00 | 51.79 | C |
| ATOM | 1272 | C | GLY | F | 135 | 51.632 | 101.558 | 193.058 | 1.00 | 52.22 | C |
| ATOM | 1273 | O | GLY | F | 135 | 50.600 | 102.230 | 193.062 | 1.00 | 52.19 | O |
| ATOM | 1275 | N | ARG | F | 136 | 52.671 | 101.849 | 192.281 | 1.00 | 50.66 | N |
| ATOM | 1276 | CA | ARG | F | 136 | 52.781 | 103.109 | 191.559 | 1.00 | 47.14 | C |
| ATOM | 1278 | CB | ARG | F | 136 | 54.094 | 103.791 | 191.954 | 1.00 | 48.57 | C |
| ATOM | 1281 | CG | ARG | F | 136 | 54.170 | 104.184 | 193.424 | 1.00 | 49.69 | C |
| ATOM | 1284 | CD | ARG | F | 136 | 53.239 | 105.351 | 193.735 | 1.00 | 48.83 | C |
| ATOM | 1287 | NE | ARG | F | 136 | 53.213 | 105.678 | 195.155 | 1.00 | 48.94 | N |
| ATOM | 1289 | CZ | ARG | F | 136 | 52.465 | 105.070 | 196.075 | 1.00 | 48.61 | C |
| ATOM | 1290 | NH1 | ARG | F | 136 | 51.652 | 104.070 | 195.750 | 1.00 | 47.33 | N |
| ATOM | 1293 | NH2 | ARG | F | 136 | 52.535 | 105.469 | 197.344 | 1.00 | 47.54 | N |
| ATOM | 1296 | C | ARG | F | 136 | 52.742 | 102.947 | 190.048 | 1.00 | 42.89 | C |
| ATOM | 1297 | O | ARG | F | 136 | 52.679 | 103.939 | 189.330 | 1.00 | 36.01 | O |
| ATOM | 1299 | N | ARG | F | 137 | 52.748 | 101.705 | 189.565 | 1.00 | 41.10 | N |
| ATOM | 1300 | CA | ARG | F | 137 | 53.078 | 101.446 | 188.176 | 1.00 | 38.01 | C |
| ATOM | 1302 | CB | ARG | F | 137 | 54.454 | 100.790 | 188.095 | 1.00 | 41.95 | C |
| ATOM | 1305 | CG | ARG | F | 137 | 55.381 | 101.461 | 187.126 | 1.00 | 48.06 | C |
| ATOM | 1308 | CD | ARG | F | 137 | 56.173 | 100.473 | 186.305 | 1.00 | 48.97 | C |
| ATOM | 1311 | NE | ARG | F | 137 | 57.188 | 99.779 | 187.067 | 1.00 | 51.07 | N |
| ATOM | 1313 | CZ | ARG | F | 137 | 58.211 | 99.124 | 186.525 | 1.00 | 47.02 | C |
| ATOM | 1314 | NH1 | ARG | F | 137 | 58.375 | 99.083 | 185.204 | 1.00 | 43.96 | N |
| ATOM | 1317 | NH2 | ARG | F | 137 | 59.070 | 98.518 | 187.320 | 1.00 | 43.53 | N |
| ATOM | 1320 | C | ARG | F | 137 | 52.079 | 100.547 | 187.468 | 1.00 | 32.66 | C |
| ATOM | 1321 | O | ARG | F | 137 | 51.494 | 99.646 | 188.075 | 1.00 | 31.68 | O |
| ATOM | 1323 | N | ILE | F | 138 | 51.915 | 100.790 | 186.173 | 1.00 | 30.74 | N |
| ATOM | 1324 | CA | ILE | F | 138 | 51.198 | 99.876 | 185.295 | 1.00 | 31.14 | C |
| ATOM | 1326 | CB | ILE | F | 138 | 49.959 | 100.545 | 184.687 | 1.00 | 29.86 | C |
| ATOM | 1328 | CG1 | ILE | F | 138 | 48.950 | 100.784 | 185.812 | 1.00 | 32.61 | C |
| ATOM | 1331 | CD1 | ILE | F | 138 | 47.735 | 101.487 | 185.417 | 1.00 | 33.80 | C |
| ATOM | 1335 | CG2 | ILE | F | 138 | 49.359 | 99.687 | 183.561 | 1.00 | 28.66 | C |
| ATOM | 1339 | C | ILE | F | 138 | 52.139 | 99.418 | 184.202 | 1.00 | 31.42 | C |
| ATOM | 1340 | O | ILE | F | 138 | 52.832 | 100.224 | 183.596 | 1.00 | 29.29 | O |
| ATOM | 1342 | N | VAL | F | 139 | 52.181 | 98.109 | 183.992 | 1.00 | 29.60 | N |
| ATOM | 1343 | CA | VAL | F | 139 | 52.813 | 97.501 | 182.823 | 1.00 | 35.14 | C |
| ATOM | 1345 | CB | VAL | F | 139 | 54.195 | 96.897 | 183.144 | 1.00 | 42.11 | C |
| ATOM | 1347 | CG1 | VAL | F | 139 | 54.832 | 96.349 | 181.874 | 1.00 | 44.18 | C |
| ATOM | 1351 | CG2 | VAL | F | 139 | 55.112 | 97.931 | 183.776 | 1.00 | 42.63 | C |
| ATOM | 1355 | C | VAL | F | 139 | 51.865 | 96.384 | 182.392 | 1.00 | 36.13 | C |
| ATOM | 1356 | O | VAL | F | 139 | 51.858 | 95.316 | 182.986 | 1.00 | 42.25 | O |
| ATOM | 1358 | N | PHE | F | 140 | 51.027 | 96.658 | 181.403 | 1.00 | 34.44 | N |
| ATOM | 1359 | CA | PHE | F | 140 | 49.879 | 95.795 | 181.129 | 1.00 | 33.21 | C |
| ATOM | 1361 | CB | PHE | F | 140 | 48.595 | 96.485 | 181.603 | 1.00 | 35.89 | C |
| ATOM | 1364 | CG | PHE | F | 140 | 47.356 | 95.668 | 181.392 | 1.00 | 36.81 | C |
| ATOM | 1365 | CD1 | PHE | F | 140 | 47.108 | 94.560 | 182.184 | 1.00 | 36.71 | C |
| ATOM | 1367 | CE1 | PHE | F | 140 | 45.968 | 93.801 | 182.007 | 1.00 | 37.36 | C |
| ATOM | 1369 | CZ | PHE | F | 140 | 45.069 | 94.136 | 181.031 | 1.00 | 37.64 | C |
| ATOM | 1371 | CE2 | PHE | F | 140 | 45.301 | 95.241 | 180.217 | 1.00 | 39.99 | C |
| ATOM | 1373 | CD2 | PHE | F | 140 | 46.445 | 96.001 | 180.403 | 1.00 | 40.03 | C |
| ATOM | 1375 | C | PHE | F | 140 | 49.762 | 95.448 | 179.655 | 1.00 | 28.19 | C |
| ATOM | 1376 | O | PHE | F | 140 | 49.782 | 96.333 | 178.804 | 1.00 | 28.51 | O |
| ATOM | 1378 | N | THR | F | 141 | 49.629 | 94.159 | 179.364 | 1.00 | 29.73 | N |
| ATOM | 1379 | CA | THR | F | 141 | 49.434 | 93.671 | 178.003 | 1.00 | 31.81 | C |
| ATOM | 1381 | CB | THR | F | 141 | 50.598 | 92.760 | 177.570 | 1.00 | 39.40 | C |
| ATOM | 1383 | OG1 | THR | F | 141 | 51.836 | 93.443 | 177.758 | 1.00 | 39.39 | O |
| ATOM | 1385 | CG2 | THR | F | 141 | 50.452 | 92.352 | 176.104 | 1.00 | 40.26 | C |
| ATOM | 1389 | C | THR | F | 141 | 48.162 | 92.832 | 177.913 | 1.00 | 31.07 | C |
| ATOM | 1390 | O | THR | F | 141 | 47.933 | 91.943 | 178.737 | 1.00 | 33.43 | O |
| ATOM | 1392 | N | VAL | F | 142 | 47.334 | 93.126 | 176.919 | 1.00 | 29.06 | N |
| ATOM | 1393 | CA | VAL | F | 142 | 46.192 | 92.264 | 176.567 | 1.00 | 32.68 | C |
| ATOM | 1395 | CB | VAL | F | 142 | 44.825 | 92.883 | 177.013 | 1.00 | 34.56 | C |
| ATOM | 1397 | CG1 | VAL | F | 142 | 44.595 | 94.208 | 176.401 | 1.00 | 41.46 | C |
| ATOM | 1401 | CG2 | VAL | F | 142 | 43.647 | 91.952 | 176.679 | 1.00 | 39.57 | C |
| ATOM | 1405 | C | VAL | F | 142 | 46.188 | 91.987 | 175.064 | 1.00 | 29.71 | C |
| ATOM | 1406 | O | VAL | F | 142 | 46.550 | 92.841 | 174.271 | 1.00 | 30.24 | O |
| ATOM | 1408 | N | VAL | F | 143 | 45.783 | 90.781 | 174.687 | 1.00 | 30.43 | N |
| ATOM | 1409 | CA | VAL | F | 143 | 45.454 | 90.467 | 173.306 | 1.00 | 26.34 | C |
| ATOM | 1411 | CB | VAL | F | 143 | 46.202 | 89.222 | 172.775 | 1.00 | 28.05 | C |
| ATOM | 1413 | CG1 | VAL | F | 143 | 45.916 | 89.025 | 171.288 | 1.00 | 28.61 | C |
| ATOM | 1417 | CG2 | VAL | F | 143 | 47.688 | 89.340 | 173.025 | 1.00 | 32.10 | C |
| ATOM | 1421 | C | VAL | F | 143 | 43.952 | 90.234 | 173.279 | 1.00 | 28.54 | C |
| ATOM | 1422 | O | VAL | F | 143 | 43.418 | 89.499 | 174.117 | 1.00 | 29.57 | O |
| ATOM | 1424 | N | ALA | F | 144 | 43.283 | 90.873 | 172.332 | 1.00 | 30.16 | N |
| ATOM | 1425 | CA | ALA | F | 144 | 41.828 | 90.853 | 172.252 | 1.00 | 30.65 | C |
| ATOM | 1427 | CB | ALA | F | 144 | 41.260 | 92.067 | 172.987 | 1.00 | 32.27 | C |
| ATOM | 1431 | C | ALA | F | 144 | 41.357 | 90.855 | 170.805 | 1.00 | 29.58 | C |
| ATOM | 1432 | O | ALA | F | 144 | 42.003 | 91.443 | 169.925 | 1.00 | 28.90 | O |
| ATOM | 1434 | N | SER | F | 145 | 40.221 | 90.207 | 170.564 | 1.00 | 29.16 | N |
| ATOM | 1435 | CA | SER | F | 145 | 39.550 | 90.279 | 169.275 | 1.00 | 31.00 | C |
| ATOM | 1437 | CB | SER | F | 145 | 38.658 | 89.055 | 169.043 | 1.00 | 32.81 | C |
| ATOM | 1440 | OG | SER | F | 145 | 38.092 | 89.092 | 167.747 | 1.00 | 36.52 | O |
| ATOM | 1442 | C | SER | F | 145 | 38.699 | 91.549 | 169.243 | 1.00 | 32.33 | C |
| ATOM | 1443 | O | SER | F | 145 | 37.755 | 91.687 | 170.013 | 1.00 | 32.56 | O |
| ATOM | 1445 | N | LEU | F | 146 | 39.062 | 92.478 | 168.368 | 1.00 | 28.60 | N |
| ATOM | 1446 | CA | LEU | F | 146 | 38.328 | 93.716 | 168.190 | 1.00 | 30.06 | C |
| ATOM | 1448 | CB | LEU | F | 146 | 39.243 | 94.926 | 168.354 | 1.00 | 29.79 | C |
| ATOM | 1451 | CG | LEU | F | 146 | 40.044 | 95.014 | 169.659 | 1.00 | 29.76 | C |
| ATOM | 1453 | CD1 | LEU | F | 146 | 40.896 | 96.283 | 169.642 | 1.00 | 30.68 | C |
| ATOM | 1457 | CD2 | LEU | F | 146 | 39.129 | 94.997 | 170.861 | 1.00 | 31.15 | C |
| ATOM | 1461 | C | LEU | F | 146 | 37.665 | 93.748 | 166.820 | 1.00 | 30.65 | C |
| ATOM | 1462 | O | LEU | F | 146 | 38.064 | 93.038 | 165.901 | 1.00 | 30.30 | O |
| ATOM | 1464 | N | ALA | F | 147 | 36.644 | 94.587 | 166.701 | 1.00 | 29.76 | N |
| ATOM | 1465 | CA | ALA | F | 147 | 35.865 | 94.694 | 165.471 | 1.00 | 31.78 | C |
| ATOM | 1467 | CB | ALA | F | 147 | 34.576 | 93.871 | 165.588 | 1.00 | 31.54 | C |
| ATOM | 1471 | C | ALA | F | 147 | 35.533 | 96.141 | 165.164 | 1.00 | 30.56 | C |
| ATOM | 1472 | O | ALA | F | 147 | 35.511 | 96.981 | 166.060 | 1.00 | 32.11 | O |
| ATOM | 1474 | N | PHE | F | 148 | 35.282 | 96.430 | 163.892 | 1.00 | 30.23 | N |
| ATOM | 1475 | CA | PHE | F | 148 | 34.700 | 97.714 | 163.490 | 1.00 | 29.13 | C |
| ATOM | 1477 | CB | PHE | F | 148 | 34.182 | 97.636 | 162.046 | 1.00 | 27.59 | C |
| ATOM | 1480 | CG | PHE | F | 148 | 33.390 | 98.837 | 161.606 | 1.00 | 28.92 | C |
| ATOM | 1481 | CD1 | PHE | F | 148 | 33.914 | 100.119 | 161.730 | 1.00 | 26.93 | C |
| ATOM | 1483 | CE1 | PHE | F | 148 | 33.196 | 101.218 | 161.313 | 1.00 | 27.53 | C |
| ATOM | 1485 | CZ | PHE | F | 148 | 31.946 | 101.063 | 160.757 | 1.00 | 28.80 | C |
| ATOM | 1487 | CE2 | PHE | F | 148 | 31.408 | 99.783 | 160.608 | 1.00 | 29.05 | C |
| ATOM | 1489 | CD2 | PHE | F | 148 | 32.132 | 98.685 | 161.033 | 1.00 | 27.28 | C |
| ATOM | 1491 | C | PHE | F | 148 | 33.573 | 98.112 | 164.445 | 1.00 | 30.68 | C |
| ATOM | 1492 | O | PHE | F | 148 | 32.741 | 97.288 | 164.812 | 1.00 | 32.23 | O |
| ATOM | 1494 | N | LYS | F | 149 | 33.569 | 99.389 | 164.824 | 1.00 | 32.61 | N |
| ATOM | 1495 | CA | LYS | F | 149 | 32.645 | 99.997 | 165.796 | 1.00 | 29.94 | C |
| ATOM | 1497 | CB | LYS | F | 149 | 31.214 | 99.442 | 165.721 | 1.00 | 32.26 | C |
| ATOM | 1500 | CG | LYS | F | 149 | 30.525 | 99.750 | 164.398 | 1.00 | 28.97 | C |
| ATOM | 1503 | CD | LYS | F | 149 | 29.103 | 99.261 | 164.367 | 1.00 | 29.28 | C |
| ATOM | 1506 | CE | LYS | F | 149 | 28.554 | 99.307 | 162.956 | 1.00 | 30.08 | C |
| ATOM | 1509 | NZ | LYS | F | 149 | 27.105 | 99.022 | 162.918 | 1.00 | 27.52 | N |
| ATOM | 1513 | C | LYS | F | 149 | 33.181 | 99.984 | 167.231 | 1.00 | 32.24 | C |
| ATOM | 1514 | O | LYS | F | 149 | 32.651 | 100.676 | 168.093 | 1.00 | 25.91 | O |
| ATOM | 1516 | N | ASP | F | 150 | 34.240 | 99.220 | 167.492 | 1.00 | 32.13 | N |
| ATOM | 1517 | CA | ASP | F | 150 | 34.862 | 99.252 | 168.808 | 1.00 | 32.81 | C |
| ATOM | 1519 | CB | ASP | F | 150 | 35.824 | 98.071 | 169.013 | 1.00 | 32.80 | C |
| ATOM | 1522 | CG | ASP | F | 150 | 35.102 | 96.728 | 169.223 | 1.00 | 35.26 | C |
| ATOM | 1523 | OD1 | ASP | F | 150 | 33.874 | 96.694 | 169.474 | 1.00 | 33.22 | O |
| ATOM | 1524 | OD2 | ASP | F | 150 | 35.785 | 95.687 | 169.149 | 1.00 | 33.07 | O |
| ATOM | 1525 | C | ASP | F | 150 | 35.585 | 100.594 | 168.991 | 1.00 | 32.78 | C |
| ATOM | 1526 | O | ASP | F | 150 | 36.008 | 101.234 | 168.022 | 1.00 | 30.42 | O |
| ATOM | 1528 | N | LYS | F | 151 | 35.683 | 101.032 | 170.241 | 1.00 | 35.98 | N |
| ATOM | 1529 | CA | LYS | F | 151 | 36.393 | 102.260 | 170.594 | 1.00 | 35.80 | C |
| ATOM | 1531 | CB | LYS | F | 151 | 35.408 | 103.430 | 170.742 | 1.00 | 39.98 | C |
| ATOM | 1534 | CG | LYS | F | 151 | 34.879 | 103.951 | 169.400 | 1.00 | 41.76 | C |
| ATOM | 1537 | CD | LYS | F | 151 | 33.683 | 104.869 | 169.554 | 1.00 | 43.66 | C |
| ATOM | 1540 | CE | LYS | F | 151 | 33.134 | 105.292 | 168.194 | 1.00 | 43.97 | C |
| ATOM | 1543 | NZ | LYS | F | 151 | 31.870 | 106.082 | 168.327 | 1.00 | 47.14 | N |
| ATOM | 1547 | C | LYS | F | 151 | 37.137 | 101.983 | 171.895 | 1.00 | 33.57 | C |
| ATOM | 1548 | O | LYS | F | 151 | 36.511 | 101.678 | 172.909 | 1.00 | 33.07 | O |
| ATOM | 1550 | N | VAL | F | 152 | 38.468 | 102.044 | 171.852 | 1.00 | 30.79 | N |
| ATOM | 1551 | CA | VAL | F | 152 | 39.304 | 101.676 | 172.996 | 1.00 | 30.67 | C |
| ATOM | 1553 | CB | VAL | F | 152 | 40.643 | 101.031 | 172.544 | 1.00 | 31.45 | C |
| ATOM | 1555 | CG1 | VAL | F | 152 | 41.529 | 100.687 | 173.750 | 1.00 | 35.17 | C |
| ATOM | 1559 | CG2 | VAL | F | 152 | 40.379 | 99.776 | 171.698 | 1.00 | 34.71 | C |
| ATOM | 1563 | C | VAL | F | 152 | 39.596 | 102.904 | 173.846 | 1.00 | 29.54 | C |
| ATOM | 1564 | O | VAL | F | 152 | 40.146 | 103.885 | 173.348 | 1.00 | 30.16 | O |
| ATOM | 1566 | N | TYR | F | 153 | 39.222 | 102.850 | 175.121 | 1.00 | 29.57 | N |
| ATOM | 1567 | CA | TYR | F | 153 | 39.481 | 103.959 | 176.043 | 1.00 | 30.80 | C |
| ATOM | 1569 | CB | TYR | F | 153 | 38.374 | 105.015 | 175.957 | 1.00 | 31.78 | C |
| ATOM | 1572 | CG | TYR | F | 153 | 37.000 | 104.520 | 176.331 | 1.00 | 31.12 | C |
| ATOM | 1573 | CD1 | TYR | F | 153 | 36.202 | 103.857 | 175.404 | 1.00 | 32.46 | C |
| ATOM | 1575 | CE1 | TYR | F | 153 | 34.931 | 103.397 | 175.748 | 1.00 | 32.60 | C |
| ATOM | 1577 | CZ | TYR | F | 153 | 34.452 | 103.612 | 177.024 | 1.00 | 31.96 | C |
| ATOM | 1578 | OH | TYR | F | 153 | 33.196 | 103.168 | 177.376 | 1.00 | 33.94 | O |
| ATOM | 1580 | CE2 | TYR | F | 153 | 35.227 | 104.277 | 177.956 | 1.00 | 32.37 | C |
| ATOM | 1582 | CD2 | TYR | F | 153 | 36.492 | 104.726 | 177.606 | 1.00 | 30.63 | C |
| ATOM | 1584 | C | TYR | F | 153 | 39.680 | 103.470 | 177.470 | 1.00 | 28.88 | C |
| ATOM | 1585 | O | TYR | F | 153 | 39.434 | 102.303 | 177.780 | 1.00 | 30.97 | O |
| ATOM | 1587 | N | LEU | F | 154 | 40.153 | 104.366 | 178.329 | 1.00 | 27.30 | N |
| ATOM | 1588 | CA | LEU | F | 154 | 40.544 | 104.002 | 179.680 | 1.00 | 29.52 | C |
| ATOM | 1590 | CB | LEU | F | 154 | 42.058 | 104.094 | 179.809 | 1.00 | 31.10 | C |
| ATOM | 1593 | CG | LEU | F | 154 | 42.856 | 103.153 | 178.899 | 1.00 | 34.02 | C |
| ATOM | 1595 | CD1 | LEU | F | 154 | 44.076 | 103.873 | 178.371 | 1.00 | 38.13 | C |
| ATOM | 1599 | CD2 | LEU | F | 154 | 43.238 | 101.881 | 179.632 | 1.00 | 35.46 | C |
| ATOM | 1603 | C | LEU | F | 154 | 39.889 | 104.934 | 180.681 | 1.00 | 28.29 | C |
| ATOM | 1604 | O | LEU | F | 154 | 40.003 | 106.141 | 180.559 | 1.00 | 26.74 | O |
| ATOM | 1606 | N | THR | F | 155 | 39.203 | 104.368 | 181.670 | 1.00 | 27.72 | N |
| ATOM | 1607 | CA | THR | F | 155 | 38.606 | 105.167 | 182.732 | 1.00 | 29.68 | C |
| ATOM | 1609 | CB | THR | F | 155 | 37.128 | 104.794 | 182.988 | 1.00 | 30.42 | C |
| ATOM | 1611 | OG1 | THR | F | 155 | 37.022 | 103.397 | 183.271 | 1.00 | 29.41 | O |
| ATOM | 1613 | CG2 | THR | F | 155 | 36.272 | 105.130 | 181.790 | 1.00 | 31.00 | C |
| ATOM | 1617 | C | THR | F | 155 | 39.388 | 105.003 | 184.027 | 1.00 | 30.72 | C |
| ATOM | 1618 | O | THR | F | 155 | 40.087 | 104.009 | 184.231 | 1.00 | 30.95 | O |
| ATOM | 1620 | N | VAL | F | 156 | 39.281 | 106.008 | 184.885 | 1.00 | 31.97 | N |
| ATOM | 1621 | CA | VAL | F | 156 | 39.828 | 105.960 | 186.227 | 1.00 | 37.73 | C |
| ATOM | 1623 | CB | VAL | F | 156 | 40.626 | 107.230 | 186.541 | 1.00 | 41.50 | C |
| ATOM | 1625 | CG1 | VAL | F | 156 | 41.135 | 107.204 | 187.976 | 1.00 | 42.50 | C |
| ATOM | 1629 | CG2 | VAL | F | 156 | 41.781 | 107.379 | 185.549 | 1.00 | 40.22 | C |
| ATOM | 1633 | C | VAL | F | 156 | 38.648 | 105.842 | 187.188 | 1.00 | 40.99 | C |
| ATOM | 1634 | O | VAL | F | 156 | 37.727 | 106.662 | 187.141 | 1.00 | 39.99 | O |
| ATOM | 1636 | N | ASN | F | 157 | 38.659 | 104.813 | 188.036 | 1.00 | 42.25 | N |
| ATOM | 1637 | CA | ASN | F | 157 | 37.615 | 104.651 | 189.045 | 1.00 | 43.66 | C |
| ATOM | 1639 | CB | ASN | F | 157 | 37.363 | 103.167 | 189.349 | 1.00 | 46.30 | C |
| ATOM | 1642 | CG | ASN | F | 157 | 35.950 | 102.906 | 189.866 | 1.00 | 50.35 | C |
| ATOM | 1643 | OD1 | ASN | F | 157 | 35.192 | 102.129 | 189.276 | 1.00 | 56.96 | O |
| ATOM | 1644 | ND2 | ASN | F | 157 | 35.587 | 103.568 | 190.959 | 1.00 | 50.63 | N |
| ATOM | 1647 | C | ASN | F | 157 | 38.004 | 105.431 | 190.305 | 1.00 | 43.69 | C |
| ATOM | 1648 | O | ASN | F | 157 | 38.716 | 104.920 | 191.173 | 1.00 | 43.76 | O |
| ATOM | 1650 | N | ALA | F | 158 | 37.545 | 106.678 | 190.380 | 1.00 | 44.97 | N |
| ATOM | 1651 | CA | ALA | F | 158 | 37.864 | 107.570 | 191.497 | 1.00 | 45.39 | C |
| ATOM | 1653 | CB | ALA | F | 158 | 39.341 | 107.942 | 191.461 | 1.00 | 46.60 | C |
| ATOM | 1657 | C | ALA | F | 158 | 37.000 | 108.835 | 191.436 | 1.00 | 46.23 | C |
| ATOM | 1658 | O | ALA | F | 158 | 36.401 | 109.119 | 190.396 | 1.00 | 43.53 | O |
| ATOM | 1660 | N | PRO | F | 159 | 36.934 | 109.602 | 192.546 | 1.00 | 47.82 | N |
| ATOM | 1661 | CA | PRO | F | 159 | 36.196 | 110.871 | 192.535 | 1.00 | 48.95 | C |
| ATOM | 1663 | CB | PRO | F | 159 | 36.365 | 111.399 | 193.969 | 1.00 | 50.06 | C |
| ATOM | 1666 | CG | PRO | F | 159 | 36.758 | 110.222 | 194.786 | 1.00 | 49.84 | C |
| ATOM | 1669 | CD | PRO | F | 159 | 37.524 | 109.323 | 193.869 | 1.00 | 49.11 | C |
| ATOM | 1672 | C | PRO | F | 159 | 36.752 | 111.881 | 191.527 | 1.00 | 49.87 | C |
| ATOM | 1673 | O | PRO | F | 159 | 37.933 | 111.823 | 191.180 | 1.00 | 52.64 | O |
| ATOM | 1674 | N | ASP | F | 160 | 35.902 | 112.797 | 191.070 | 1.00 | 49.94 | N |
| ATOM | 1675 | CA | ASP | F | 160 | 36.282 | 113.794 | 190.059 | 1.00 | 51.49 | C |
| ATOM | 1677 | CB | ASP | F | 160 | 35.151 | 114.817 | 189.865 | 1.00 | 54.24 | C |
| ATOM | 1680 | CG | ASP | F | 160 | 33.907 | 114.210 | 189.221 | 1.00 | 55.84 | C |
| ATOM | 1681 | OD1 | ASP | F | 160 | 34.044 | 113.498 | 188.206 | 1.00 | 55.57 | O |
| ATOM | 1682 | OD2 | ASP | F | 160 | 32.791 | 114.455 | 189.730 | 1.00 | 56.57 | O |
| ATOM | 1683 | C | ASP | F | 160 | 37.578 | 114.532 | 190.413 | 1.00 | 51.05 | C |
| ATOM | 1684 | O | ASP | F | 160 | 38.418 | 114.772 | 189.546 | 1.00 | 48.96 | O |
| ATOM | 1686 | N | THR | F | 161 | 37.725 | 114.882 | 191.689 | 1.00 | 51.67 | N |
| ATOM | 1687 | CA | THR | F | 161 | 38.886 | 115.629 | 192.181 | 1.00 | 50.62 | C |
| ATOM | 1689 | CB | THR | F | 161 | 38.745 | 115.919 | 193.686 | 1.00 | 51.31 | C |
| ATOM | 1691 | OG1 | THR | F | 161 | 37.591 | 116.738 | 193.901 | 1.00 | 53.82 | O |
| ATOM | 1693 | CG2 | THR | F | 161 | 39.977 | 116.634 | 194.230 | 1.00 | 52.05 | C |
| ATOM | 1697 | C | THR | F | 161 | 40.210 | 114.903 | 191.947 | 1.00 | 48.52 | C |
| ATOM | 1698 | O | THR | F | 161 | 41.201 | 115.528 | 191.566 | 1.00 | 47.96 | O |
| ATOM | 1700 | N | LEU | F | 162 | 40.225 | 113.597 | 192.199 | 1.00 | 47.49 | N |
| ATOM | 1701 | CA | LEU | F | 162 | 41.423 | 112.786 | 191.999 | 1.00 | 47.25 | C |
| ATOM | 1703 | CB | LEU | F | 162 | 41.178 | 111.336 | 192.444 | 1.00 | 48.04 | C |
| ATOM | 1706 | CG | LEU | F | 162 | 42.362 | 110.349 | 192.397 | 1.00 | 48.96 | C |
| ATOM | 1708 | CD1 | LEU | F | 162 | 42.208 | 109.263 | 193.463 | 1.00 | 50.01 | C |
| ATOM | 1712 | CD2 | LEU | F | 162 | 42.543 | 109.710 | 191.022 | 1.00 | 49.97 | C |
| ATOM | 1716 | C | LEU | F | 162 | 41.872 | 112.836 | 190.536 | 1.00 | 46.49 | C |
| ATOM | 1717 | O | LEU | F | 162 | 43.059 | 113.006 | 190.264 | 1.00 | 44.88 | O |
| ATOM | 1719 | N | CYS | F | 163 | 40.919 | 112.692 | 189.612 | 1.00 | 45.37 | N |
| ATOM | 1720 | CA | CYS | F | 163 | 41.195 | 112.747 | 188.168 | 1.00 | 43.21 | C |
| ATOM | 1722 | CB | CYS | F | 163 | 39.919 | 112.450 | 187.361 | 1.00 | 46.10 | C |
| ATOM | 1725 | SG | CYS | F | 163 | 40.066 | 112.761 | 185.566 | 1.00 | 48.69 | S |
| ATOM | 1727 | C | CYS | F | 163 | 41.788 | 114.087 | 187.726 | 1.00 | 39.68 | C |
| ATOM | 1728 | O | CYS | F | 163 | 42.794 | 114.118 | 187.027 | 1.00 | 36.24 | O |
| ATOM | 1730 | N | GLU | F | 164 | 41.183 | 115.191 | 188.163 | 1.00 | 40.04 | N |
| ATOM | 1731 | CA | GLU | F | 164 | 41.630 | 116.531 | 187.762 | 1.00 | 39.02 | C |
| ATOM | 1733 | CB | GLU | F | 164 | 40.729 | 117.605 | 188.380 | 1.00 | 43.22 | C |
| ATOM | 1736 | CG | GLU | F | 164 | 39.305 | 117.605 | 187.857 | 1.00 | 45.05 | C |
| ATOM | 1739 | CD | GLU | F | 164 | 38.396 | 118.515 | 188.654 | 1.00 | 46.26 | C |
| ATOM | 1740 | OE1 | GLU | F | 164 | 38.880 | 119.557 | 189.151 | 1.00 | 49.45 | O |
| ATOM | 1741 | OE2 | GLU | F | 164 | 37.195 | 118.186 | 188.784 | 1.00 | 50.36 | O |
| ATOM | 1742 | C | GLU | F | 164 | 43.084 | 116.822 | 188.150 | 1.00 | 35.83 | C |
| ATOM | 1743 | O | GLU | F | 164 | 43.780 | 117.563 | 187.454 | 1.00 | 35.73 | O |
| ATOM | 1745 | N | HIS | F | 165 | 43.530 | 116.245 | 189.263 | 1.00 | 32.13 | N |
| ATOM | 1746 | CA | HIS | F | 165 | 44.874 | 116.490 | 189.783 | 1.00 | 32.56 | C |
| ATOM | 1748 | CB | HIS | F | 165 | 44.797 | 116.787 | 191.282 | 1.00 | 33.94 | C |
| ATOM | 1751 | CG | HIS | F | 165 | 43.907 | 117.945 | 191.617 | 1.00 | 32.55 | C |
| ATOM | 1752 | ND1 | HIS | F | 165 | 43.965 | 119.144 | 190.939 | 1.00 | 35.41 | N |
| ATOM | 1754 | CE1 | HIS | F | 165 | 43.068 | 119.975 | 191.441 | 1.00 | 36.93 | C |
| ATOM | 1756 | NE2 | HIS | F | 165 | 42.428 | 119.355 | 192.417 | 1.00 | 37.11 | N |
| ATOM | 1758 | CD2 | HIS | F | 165 | 42.931 | 118.082 | 192.543 | 1.00 | 33.76 | C |
| ATOM | 1760 | C | HIS | F | 165 | 45.847 | 115.333 | 189.525 | 1.00 | 30.15 | C |
| ATOM | 1761 | O | HIS | F | 165 | 47.001 | 115.400 | 189.938 | 1.00 | 30.52 | O |
| ATOM | 1763 | N | LEU | F | 166 | 45.391 | 114.292 | 188.832 | 1.00 | 30.38 | N |
| ATOM | 1764 | CA | LEU | F | 166 | 46.224 | 113.116 | 188.566 | 1.00 | 30.63 | C |
| ATOM | 1766 | CB | LEU | F | 166 | 45.393 | 111.977 | 187.958 | 1.00 | 32.97 | C |
| ATOM | 1769 | CG | LEU | F | 166 | 46.036 | 110.582 | 187.972 | 1.00 | 34.63 | C |
| ATOM | 1771 | CD1 | LEU | F | 166 | 46.438 | 110.173 | 189.391 | 1.00 | 35.80 | C |
| ATOM | 1775 | CD2 | LEU | F | 166 | 45.098 | 109.538 | 187.370 | 1.00 | 35.74 | C |
| ATOM | 1779 | C | LEU | F | 166 | 47.391 | 113.472 | 187.640 | 1.00 | 30.85 | C |
| ATOM | 1780 | O | LEU | F | 166 | 47.195 | 114.091 | 186.585 | 1.00 | 32.27 | O |
| ATOM | 1782 | N | GLN | F | 167 | 48.601 | 113.100 | 188.060 | 1.00 | 26.54 | N |
| ATOM | 1783 | CA | GLN | F | 167 | 49.815 | 113.323 | 187.282 | 1.00 | 27.54 | C |
| ATOM | 1785 | CB | GLN | F | 167 | 50.830 | 114.132 | 188.100 | 1.00 | 26.85 | C |
| ATOM | 1788 | CG | GLN | F | 167 | 50.322 | 115.517 | 188.507 | 1.00 | 29.90 | C |
| ATOM | 1791 | CD | GLN | F | 167 | 51.321 | 116.311 | 189.324 | 1.00 | 31.20 | C |
| ATOM | 1792 | OE1 | GLN | F | 167 | 52.256 | 115.759 | 189.896 | 1.00 | 32.29 | O |
| ATOM | 1793 | NE2 | GLN | F | 167 | 51.107 | 117.617 | 189.406 | 1.00 | 38.56 | N |
| ATOM | 1796 | C | GLN | F | 167 | 50.393 | 111.972 | 186.879 | 1.00 | 30.20 | C |
| ATOM | 1797 | O | GLN | F | 167 | 50.560 | 111.091 | 187.727 | 1.00 | 30.34 | O |
| ATOM | 1799 | N | ILE | F | 168 | 50.666 | 111.797 | 185.586 | 1.00 | 28.45 | N |
| ATOM | 1800 | CA | ILE | F | 168 | 51.175 | 110.528 | 185.059 | 1.00 | 28.43 | C |
| ATOM | 1802 | CB | ILE | F | 168 | 50.156 | 109.874 | 184.084 | 1.00 | 30.51 | C |
| ATOM | 1804 | CG1 | ILE | F | 168 | 48.757 | 109.862 | 184.724 | 1.00 | 33.96 | C |
| ATOM | 1807 | CD1 | ILE | F | 168 | 47.648 | 109.447 | 183.798 | 1.00 | 34.62 | C |
| ATOM | 1811 | CG2 | ILE | F | 168 | 50.607 | 108.464 | 183.683 | 1.00 | 32.63 | C |
| ATOM | 1815 | C | ILE | F | 168 | 52.506 | 110.756 | 184.353 | 1.00 | 28.48 | C |
| ATOM | 1816 | O | ILE | F | 168 | 52.621 | 111.653 | 183.512 | 1.00 | 28.72 | O |
| ATOM | 1818 | N | ASN | F | 169 | 53.514 | 109.966 | 184.732 | 1.00 | 26.67 | N |
| ATOM | 1819 | CA | ASN | F | 169 | 54.827 | 109.959 | 184.082 | 1.00 | 29.36 | C |
| ATOM | 1821 | CB | ASN | F | 169 | 55.959 | 110.040 | 185.115 | 1.00 | 28.75 | C |
| ATOM | 1824 | CG | ASN | F | 169 | 56.247 | 111.456 | 185.586 | 1.00 | 27.48 | C |
| ATOM | 1825 | OD1 | ASN | F | 169 | 55.914 | 112.439 | 184.919 | 1.00 | 24.90 | O |
| ATOM | 1826 | ND2 | ASN | F | 169 | 56.899 | 111.564 | 186.741 | 1.00 | 23.92 | N |
| ATOM | 1829 | C | ASN | F | 169 | 54.981 | 108.655 | 183.302 | 1.00 | 32.98 | C |
| ATOM | 1830 | O | ASN | F | 169 | 54.358 | 107.654 | 183.641 | 1.00 | 32.59 | O |
| ATOM | 1832 | N | ASP | F | 170 | 55.810 | 108.673 | 182.263 | 1.00 | 38.46 | N |
| ATOM | 1833 | CA | ASP | F | 170 | 56.169 | 107.451 | 181.533 | 1.00 | 37.00 | C |
| ATOM | 1835 | CB | ASP | F | 170 | 57.027 | 106.559 | 182.448 | 1.00 | 42.36 | C |
| ATOM | 1838 | CG | ASP | F | 170 | 57.801 | 105.484 | 181.692 | 1.00 | 48.69 | C |
| ATOM | 1839 | OD1 | ASP | F | 170 | 58.112 | 105.670 | 180.493 | 1.00 | 53.95 | O |
| ATOM | 1840 | OD2 | ASP | F | 170 | 58.119 | 104.450 | 182.322 | 1.00 | 54.05 | O |
| ATOM | 1841 | C | ASP | F | 170 | 54.908 | 106.711 | 181.065 | 1.00 | 37.38 | C |
| ATOM | 1842 | O | ASP | F | 170 | 54.829 | 105.482 | 181.153 | 1.00 | 37.13 | O |
| ATOM | 1844 | N | GLY | F | 171 | 53.942 | 107.485 | 180.571 | 1.00 | 32.70 | N |
| ATOM | 1845 | CA | GLY | F | 171 | 52.604 | 107.012 | 180.255 | 1.00 | 35.98 | C |
| ATOM | 1848 | C | GLY | F | 171 | 52.426 | 106.832 | 178.761 | 1.00 | 37.54 | C |
| ATOM | 1849 | O | GLY | F | 171 | 52.657 | 107.762 | 177.987 | 1.00 | 38.58 | O |
| ATOM | 1851 | N | GLU | F | 172 | 51.999 | 105.641 | 178.355 | 1.00 | 31.57 | N |
| ATOM | 1852 | CA | GLU | F | 172 | 51.922 | 105.309 | 176.935 | 1.00 | 31.06 | C |
| ATOM | 1854 | CB | GLU | F | 172 | 53.299 | 104.882 | 176.435 | 1.00 | 30.59 | C |
| ATOM | 1857 | CG | GLU | F | 172 | 53.346 | 104.460 | 174.973 | 1.00 | 30.96 | C |
| ATOM | 1860 | CD | GLU | F | 172 | 54.757 | 104.470 | 174.441 | 1.00 | 35.33 | C |
| ATOM | 1861 | OE1 | GLU | F | 172 | 55.306 | 105.572 | 174.223 | 1.00 | 44.00 | O |
| ATOM | 1862 | OE2 | GLU | F | 172 | 55.329 | 103.396 | 174.250 | 1.00 | 38.83 | O |
| ATOM | 1863 | C | GLU | F | 172 | 50.918 | 104.204 | 176.679 | 1.00 | 29.73 | C |
| ATOM | 1864 | O | GLU | F | 172 | 50.880 | 103.208 | 177.415 | 1.00 | 30.04 | O |
| ATOM | 1866 | N | LEU | F | 173 | 50.100 | 104.406 | 175.646 | 1.00 | 29.14 | N |
| ATOM | 1867 | CA | LEU | F | 173 | 49.150 | 103.408 | 175.174 | 1.00 | 30.88 | C |
| ATOM | 1869 | CB | LEU | F | 173 | 47.724 | 103.964 | 175.181 | 1.00 | 30.65 | C |
| ATOM | 1872 | CG | LEU | F | 173 | 46.644 | 103.054 | 174.569 | 1.00 | 29.85 | C |
| ATOM | 1874 | CD1 | LEU | F | 173 | 46.356 | 101.853 | 175.472 | 1.00 | 29.90 | C |
| ATOM | 1878 | CD2 | LEU | F | 173 | 45.391 | 103.840 | 174.285 | 1.00 | 31.95 | C |
| ATOM | 1882 | C | LEU | F | 173 | 49.532 | 103.014 | 173.754 | 1.00 | 30.02 | C |
| ATOM | 1883 | O | LEU | F | 173 | 49.770 | 103.877 | 172.914 | 1.00 | 29.62 | O |
| ATOM | 1885 | N | ILE | F | 174 | 49.581 | 101.709 | 173.499 | 1.00 | 28.44 | N |
| ATOM | 1886 | CA | ILE | F | 174 | 49.781 | 101.185 | 172.159 | 1.00 | 28.40 | C |
| ATOM | 1888 | CB | ILE | F | 174 | 51.168 | 100.505 | 171.990 | 1.00 | 29.47 | C |
| ATOM | 1890 | CG1 | ILE | F | 174 | 52.271 | 101.556 | 172.024 | 1.00 | 29.90 | C |
| ATOM | 1893 | CD1 | ILE | F | 174 | 53.629 | 101.023 | 172.282 | 1.00 | 30.86 | C |
| ATOM | 1897 | CG2 | ILE | F | 174 | 51.256 | 99.748 | 170.653 | 1.00 | 29.29 | C |
| ATOM | 1901 | C | ILE | F | 174 | 48.668 | 100.202 | 171.829 | 1.00 | 29.87 | C |
| ATOM | 1902 | O | ILE | F | 174 | 48.371 | 99.296 | 172.610 | 1.00 | 28.28 | O |
| ATOM | 1904 | N | VAL | F | 175 | 48.044 | 100.408 | 170.673 | 1.00 | 30.57 | N |
| ATOM | 1905 | CA | VAL | F | 175 | 47.079 | 99.466 | 170.105 | 1.00 | 28.64 | C |
| ATOM | 1907 | CB | VAL | F | 175 | 45.701 | 100.125 | 169.925 | 1.00 | 29.59 | C |
| ATOM | 1909 | CG1 | VAL | F | 175 | 44.688 | 99.126 | 169.364 | 1.00 | 31.05 | C |
| ATOM | 1913 | CG2 | VAL | F | 175 | 45.207 | 100.687 | 171.251 | 1.00 | 31.49 | C |
| ATOM | 1917 | C | VAL | F | 175 | 47.649 | 99.046 | 168.753 | 1.00 | 27.88 | C |
| ATOM | 1918 | O | VAL | F | 175 | 47.976 | 99.900 | 167.934 | 1.00 | 30.69 | O |
| ATOM | 1920 | N | VAL | F | 176 | 47.797 | 97.748 | 168.522 | 1.00 | 28.04 | N |
| ATOM | 1921 | CA | VAL | F | 176 | 48.415 | 97.272 | 167.285 | 1.00 | 28.01 | C |
| ATOM | 1923 | CB | VAL | F | 176 | 49.975 | 97.158 | 167.424 | 1.00 | 31.82 | C |
| ATOM | 1925 | CG1 | VAL | F | 176 | 50.374 | 96.271 | 168.586 | 1.00 | 36.26 | C |
| ATOM | 1929 | CG2 | VAL | F | 176 | 50.619 | 96.693 | 166.131 | 1.00 | 31.64 | C |
| ATOM | 1933 | C | VAL | F | 176 | 47.790 | 95.948 | 166.826 | 1.00 | 29.19 | C |
| ATOM | 1934 | O | VAL | F | 176 | 47.659 | 94.998 | 167.596 | 1.00 | 26.77 | O |
| ATOM | 1936 | N | GLN | F | 177 | 47.420 | 95.929 | 165.550 | 1.00 | 28.91 | N |
| ATOM | 1937 | CA | GLN | F | 177 | 46.748 | 94.815 | 164.912 | 1.00 | 28.80 | C |
| ATOM | 1939 | CB | GLN | F | 177 | 46.121 | 95.319 | 163.616 | 1.00 | 29.08 | C |
| ATOM | 1942 | CG | GLN | F | 177 | 45.243 | 94.336 | 162.888 | 1.00 | 28.32 | C |
| ATOM | 1945 | CD | GLN | F | 177 | 44.764 | 94.877 | 161.563 | 1.00 | 28.02 | C |
| ATOM | 1946 | OE1 | GLN | F | 177 | 45.455 | 95.668 | 160.917 | 1.00 | 29.14 | O |
| ATOM | 1947 | NE2 | GLN | F | 177 | 43.574 | 94.452 | 161.144 | 1.00 | 32.31 | N |
| ATOM | 1950 | C | GLN | F | 177 | 47.737 | 93.705 | 164.620 | 1.00 | 27.77 | C |
| ATOM | 1951 | O | GLN | F | 177 | 48.826 | 93.960 | 164.101 | 1.00 | 28.33 | O |
| ATOM | 1953 | N | LEU | F | 178 | 47.352 | 92.471 | 164.944 | 1.00 | 28.40 | N |
| ATOM | 1954 | CA | LEU | F | 178 | 48.194 | 91.296 | 164.686 | 1.00 | 27.25 | C |
| ATOM | 1956 | CB | LEU | F | 178 | 48.255 | 90.403 | 165.934 | 1.00 | 29.56 | C |
| ATOM | 1959 | CG | LEU | F | 178 | 48.757 | 91.034 | 167.233 | 1.00 | 28.94 | C |
| ATOM | 1961 | CD1 | LEU | F | 178 | 48.762 | 89.994 | 168.350 | 1.00 | 29.61 | C |
| ATOM | 1965 | CD2 | LEU | F | 178 | 50.145 | 91.655 | 167.045 | 1.00 | 30.23 | C |
| ATOM | 1969 | C | LEU | F | 178 | 47.723 | 90.443 | 163.499 | 1.00 | 30.44 | C |
| ATOM | 1970 | O | LEU | F | 178 | 48.526 | 89.733 | 162.889 | 1.00 | 28.99 | O |
| ATOM | 1972 | N | THR | F | 179 | 46.426 | 90.484 | 163.201 | 1.00 | 29.98 | N |
| ATOM | 1973 | CA | THR | F | 179 | 45.847 | 89.685 | 162.126 | 1.00 | 29.50 | C |
| ATOM | 1975 | CB | THR | F | 179 | 45.109 | 88.446 | 162.666 | 1.00 | 28.80 | C |
| ATOM | 1977 | OG1 | THR | F | 179 | 44.073 | 88.860 | 163.567 | 1.00 | 33.38 | O |
| ATOM | 1979 | CG2 | THR | F | 179 | 46.065 | 87.520 | 163.380 | 1.00 | 31.06 | C |
| ATOM | 1983 | C | THR | F | 179 | 44.876 | 90.559 | 161.315 | 1.00 | 31.33 | C |
| ATOM | 1984 | O | THR | F | 179 | 44.242 | 91.477 | 161.873 | 1.00 | 29.47 | O |
| ATOM | 1986 | N | PRO | F | 180 | 44.760 | 90.287 | 160.004 | 1.00 | 33.42 | N |
| ATOM | 1987 | CA | PRO | F | 180 | 43.941 | 91.145 | 159.146 | 1.00 | 32.56 | C |
| ATOM | 1989 | CB | PRO | F | 180 | 44.449 | 90.813 | 157.739 | 1.00 | 35.67 | C |
| ATOM | 1992 | CG | PRO | F | 180 | 44.864 | 89.393 | 157.824 | 1.00 | 35.88 | C |
| ATOM | 1995 | CD | PRO | F | 180 | 45.378 | 89.185 | 159.239 | 1.00 | 34.62 | C |
| ATOM | 1998 | C | PRO | F | 180 | 42.471 | 90.828 | 159.257 | 1.00 | 34.06 | C |
| ATOM | 1999 | O | PRO | F | 180 | 42.090 | 89.770 | 159.753 | 1.00 | 31.06 | O |
| ATOM | 2000 | N | GLY | F | 181 | 41.641 | 91.750 | 158.793 | 1.00 | 33.25 | N |
| ATOM | 2001 | CA | GLY | F | 181 | 40.218 | 91.500 | 158.729 | 1.00 | 32.42 | C |
| ATOM | 2004 | C | GLY | F | 181 | 39.503 | 92.594 | 157.968 | 1.00 | 32.27 | C |
| ATOM | 2005 | O | GLY | F | 181 | 40.134 | 93.483 | 157.391 | 1.00 | 34.40 | O |
| ATOM | 2007 | N | TYR | F | 182 | 38.181 | 92.535 | 158.003 | 1.00 | 31.07 | N |
| ATOM | 2008 | CA | TYR | F | 182 | 37.330 | 93.544 | 157.389 | 1.00 | 30.52 | C |
| ATOM | 2010 | CB | TYR | F | 182 | 35.860 | 93.241 | 157.719 | 1.00 | 27.55 | C |
| ATOM | 2013 | CG | TYR | F | 182 | 34.923 | 94.410 | 157.530 | 1.00 | 26.30 | C |
| ATOM | 2014 | CD1 | TYR | F | 182 | 34.365 | 94.691 | 156.289 | 1.00 | 28.04 | C |
| ATOM | 2016 | CE1 | TYR | F | 182 | 33.514 | 95.779 | 156.117 | 1.00 | 26.36 | C |
| ATOM | 2018 | CZ | TYR | F | 182 | 33.207 | 96.582 | 157.207 | 1.00 | 25.16 | C |
| ATOM | 2019 | OH | TYR | F | 182 | 32.364 | 97.658 | 157.064 | 1.00 | 26.19 | O |
| ATOM | 2021 | CE2 | TYR | F | 182 | 33.742 | 96.316 | 158.443 | 1.00 | 26.20 | C |
| ATOM | 2023 | CD2 | TYR | F | 182 | 34.594 | 95.241 | 158.606 | 1.00 | 28.29 | C |
| ATOM | 2025 | C | TYR | F | 182 | 37.703 | 94.942 | 157.867 | 1.00 | 29.17 | C |
| ATOM | 2026 | O | TYR | F | 182 | 37.928 | 95.147 | 159.059 | 1.00 | 28.86 | O |
| ATOM | 2028 | N | CYS | F | 183 | 37.780 | 95.894 | 156.933 | 1.00 | 29.79 | N |
| ATOM | 2029 | CA | CYS | F | 183 | 37.944 | 97.316 | 157.266 | 1.00 | 31.56 | C |
| ATOM | 2031 | CB | CYS | F | 183 | 39.256 | 97.894 | 156.701 | 1.00 | 37.43 | C |
| ATOM | 2034 | SG | CYS | F | 183 | 40.812 | 97.207 | 157.355 | 1.00 | 41.45 | S |
| ATOM | 2036 | C | CYS | F | 183 | 36.775 | 98.102 | 156.690 | 1.00 | 31.70 | C |
| ATOM | 2037 | O | CYS | F | 183 | 36.415 | 97.927 | 155.525 | 1.00 | 27.60 | O |
| ATOM | 2039 | N | ALA | F | 184 | 36.178 | 98.975 | 157.495 | 1.00 | 32.41 | N |
| ATOM | 2040 | CA | ALA | F | 184 | 35.129 | 99.847 | 156.999 | 1.00 | 32.82 | C |
| ATOM | 2042 | CB | ALA | F | 184 | 34.369 | 100.456 | 158.148 | 1.00 | 30.47 | C |
| ATOM | 2046 | C | ALA | F | 184 | 35.764 | 100.951 | 156.138 | 1.00 | 34.67 | C |
| ATOM | 2047 | O | ALA | F | 184 | 36.684 | 101.615 | 156.594 | 1.00 | 38.14 | O |
| ATOM | 2049 | N | PRO | F | 185 | 35.292 | 101.136 | 154.890 | 1.00 | 38.22 | N |
| ATOM | 2050 | CA | PRO | F | 185 | 35.774 | 102.271 | 154.080 | 1.00 | 44.15 | C |
| ATOM | 2052 | CB | PRO | F | 185 | 34.901 | 102.205 | 152.824 | 1.00 | 43.58 | C |
| ATOM | 2055 | CG | PRO | F | 185 | 34.440 | 100.801 | 152.742 | 1.00 | 42.96 | C |
| ATOM | 2058 | CD | PRO | F | 185 | 34.321 | 100.308 | 154.153 | 1.00 | 40.11 | C |
| ATOM | 2061 | C | PRO | F | 185 | 35.592 | 103.622 | 154.784 | 1.00 | 48.02 | C |
| ATOM | 2062 | O | PRO | F | 185 | 34.635 | 103.793 | 155.544 | 1.00 | 48.82 | O |
| ATOM | 2063 | N | GLU | F | 186 | 36.504 | 104.565 | 154.541 | 1.00 | 53.33 | N |
| ATOM | 2064 | CA | GLU | F | 186 | 36.439 | 105.890 | 155.177 | 1.00 | 53.63 | C |
| ATOM | 2066 | CB | GLU | F | 186 | 37.654 | 106.748 | 154.786 | 1.00 | 56.08 | C |
| ATOM | 2069 | CG | GLU | F | 186 | 37.724 | 108.115 | 155.492 | 1.00 | 57.50 | C |
| ATOM | 2072 | CD | GLU | F | 186 | 38.636 | 109.117 | 154.785 | 1.00 | 59.93 | C |
| ATOM | 2073 | OE1 | GLU | F | 186 | 39.731 | 108.727 | 154.322 | 1.00 | 63.98 | O |
| ATOM | 2074 | OE2 | GLU | F | 186 | 38.256 | 110.305 | 154.699 | 1.00 | 62.77 | O |
| ATOM | 2075 | C | GLU | F | 186 | 35.147 | 106.624 | 154.804 | 1.00 | 53.10 | C |
| ATOM | 2076 | O | GLU | F | 186 | 34.740 | 106.623 | 153.640 | 1.00 | 52.75 | O |
| ATOM | 2078 | N | GLY | F | 187 | 34.510 | 107.236 | 155.803 | 1.00 | 52.60 | N |
| ATOM | 2079 | CA | GLY | F | 187 | 33.316 | 108.063 | 155.594 | 1.00 | 52.92 | C |
| ATOM | 2082 | C | GLY | F | 187 | 32.066 | 107.341 | 155.110 | 1.00 | 53.17 | C |
| ATOM | 2083 | O | GLY | F | 187 | 31.154 | 107.977 | 154.582 | 1.00 | 51.26 | O |
| ATOM | 2085 | N | SER | F | 188 | 32.007 | 106.022 | 155.301 | 1.00 | 53.46 | N |
| ATOM | 2086 | CA | SER | F | 188 | 30.895 | 105.211 | 154.782 | 1.00 | 51.73 | C |
| ATOM | 2088 | CB | SER | F | 188 | 31.437 | 103.989 | 154.029 | 1.00 | 52.30 | C |
| ATOM | 2091 | OG | SER | F | 188 | 32.223 | 103.165 | 154.879 | 1.00 | 54.21 | O |
| ATOM | 2093 | C | SER | F | 188 | 29.890 | 104.763 | 155.857 | 1.00 | 50.13 | C |
| ATOM | 2094 | O | SER | F | 188 | 28.907 | 104.095 | 155.538 | 1.00 | 49.87 | O |
| ATOM | 2096 | N | TYR | F | 189 | 30.127 | 105.123 | 157.118 | 1.00 | 47.59 | N |
| ATOM | 2097 | CA | TYR | F | 189 | 29.200 | 104.766 | 158.194 | 1.00 | 49.50 | C |
| ATOM | 2099 | CB | TYR | F | 189 | 29.617 | 103.441 | 158.861 | 1.00 | 45.16 | C |
| ATOM | 2102 | CG | TYR | F | 189 | 28.726 | 103.056 | 160.031 | 1.00 | 44.75 | C |
| ATOM | 2103 | CD1 | TYR | F | 189 | 27.552 | 102.332 | 159.831 | 1.00 | 44.79 | C |
| ATOM | 2105 | CE1 | TYR | F | 189 | 26.721 | 102.001 | 160.895 | 1.00 | 44.48 | C |
| ATOM | 2107 | CZ | TYR | F | 189 | 27.065 | 102.396 | 162.176 | 1.00 | 43.99 | C |
| ATOM | 2108 | OH | TYR | F | 189 | 26.257 | 102.066 | 163.238 | 1.00 | 43.92 | O |
| ATOM | 2110 | CE2 | TYR | F | 189 | 28.224 | 103.114 | 162.400 | 1.00 | 43.97 | C |
| ATOM | 2112 | CD2 | TYR | F | 189 | 29.044 | 103.441 | 161.331 | 1.00 | 44.52 | C |
| ATOM | 2114 | C | TYR | F | 189 | 29.085 | 105.859 | 159.257 | 1.00 | 50.32 | C |
| ATOM | 2115 | O | TYR | F | 189 | 30.086 | 106.460 | 159.655 | 1.00 | 50.46 | O |
| ATOM | 2117 | N | HIS | F | 190 | 27.856 | 106.092 | 159.717 | 1.00 | 51.52 | N |
| ATOM | 2118 | CA | HIS | F | 190 | 27.591 | 106.921 | 160.895 | 1.00 | 56.05 | C |
| ATOM | 2120 | CB | HIS | F | 190 | 27.156 | 108.332 | 160.485 | 1.00 | 61.41 | C |
| ATOM | 2123 | CG | HIS | F | 190 | 28.291 | 109.208 | 160.050 | 1.00 | 65.11 | C |
| ATOM | 2124 | ND1 | HIS | F | 190 | 28.611 | 109.411 | 158.724 | 1.00 | 66.38 | N |
| ATOM | 2126 | CE1 | HIS | F | 190 | 29.654 | 110.219 | 158.643 | 1.00 | 67.47 | C |
| ATOM | 2128 | NE2 | HIS | F | 190 | 30.024 | 110.545 | 159.869 | 1.00 | 67.73 | N |
| ATOM | 2130 | CD2 | HIS | F | 190 | 29.189 | 109.924 | 160.767 | 1.00 | 66.86 | C |
| ATOM | 2132 | C | HIS | F | 190 | 26.520 | 106.253 | 161.762 | 1.00 | 56.83 | C |
| ATOM | 2133 | O | HIS | F | 190 | 25.545 | 105.699 | 161.242 | 1.00 | 54.36 | O |
| ATOM | 2135 | N | SER | F | 191 | 26.707 | 106.309 | 163.079 | 1.00 | 58.50 | N |
| ATOM | 2136 | CA | SER | F | 191 | 25.825 | 105.617 | 164.023 | 1.00 | 59.71 | C |
| ATOM | 2138 | CB | SER | F | 191 | 26.435 | 105.620 | 165.429 | 1.00 | 61.10 | C |
| ATOM | 2141 | OG | SER | F | 191 | 25.772 | 104.852 | 166.253 | 0.00 | 30.00 | O |
| ATOM | 2143 | C | SER | F | 191 | 24.434 | 106.237 | 164.064 | 1.00 | 61.29 | C |
| ATOM | 2144 | O | SER | F | 191 | 23.469 | 105.642 | 163.583 | 1.00 | 63.22 | O |
| ATOM | 2146 | N | ASP | G | 57 | 8.975 | 62.479 | 192.445 | 1.00 | 63.34 | N |
| ATOM | 2147 | CA | ASP | G | 57 | 9.431 | 61.070 | 192.208 | 1.00 | 61.45 | C |
| ATOM | 2149 | CB | ASP | G | 57 | 10.387 | 60.983 | 190.998 | 1.00 | 64.19 | C |
| ATOM | 2152 | CG | ASP | G | 57 | 11.700 | 61.713 | 191.217 | 1.00 | 67.04 | C |
| ATOM | 2153 | OD1 | ASP | G | 57 | 12.588 | 61.164 | 191.903 | 1.00 | 69.19 | O |
| ATOM | 2154 | OD2 | ASP | G | 57 | 11.854 | 62.832 | 190.684 | 1.00 | 69.86 | O |
| ATOM | 2155 | C | ASP | G | 57 | 10.018 | 60.329 | 193.430 | 1.00 | 59.39 | C |
| ATOM | 2156 | O | ASP | G | 57 | 9.882 | 59.105 | 193.503 | 1.00 | 59.16 | O |
| ATOM | 2160 | N | PRO | G | 58 | 10.672 | 61.042 | 194.380 | 1.00 | 56.44 | N |
| ATOM | 2161 | CA | PRO | G | 58 | 11.139 | 60.298 | 195.559 | 1.00 | 51.46 | C |
| ATOM | 2163 | CB | PRO | G | 58 | 11.890 | 61.356 | 196.381 | 1.00 | 54.42 | C |
| ATOM | 2166 | CG | PRO | G | 58 | 12.185 | 62.454 | 195.430 | 1.00 | 55.83 | C |
| ATOM | 2169 | CD | PRO | G | 58 | 11.043 | 62.466 | 194.472 | 1.00 | 57.57 | C |
| ATOM | 2172 | C | PRO | G | 58 | 9.981 | 59.720 | 196.376 | 1.00 | 42.86 | C |
| ATOM | 2173 | O | PRO | G | 58 | 8.925 | 60.348 | 196.470 | 1.00 | 42.33 | O |
| ATOM | 2174 | N | PRO | G | 59 | 10.171 | 58.525 | 196.958 | 1.00 | 38.76 | N |
| ATOM | 2175 | CA | PRO | G | 59 | 9.080 | 57.899 | 197.706 | 1.00 | 38.38 | C |
| ATOM | 2177 | CB | PRO | G | 59 | 9.602 | 56.486 | 197.970 | 1.00 | 39.72 | C |
| ATOM | 2180 | CG | PRO | G | 59 | 11.085 | 56.616 | 197.944 | 1.00 | 41.19 | C |
| ATOM | 2183 | CD | PRO | G | 59 | 11.393 | 57.701 | 196.960 | 1.00 | 39.53 | C |
| ATOM | 2186 | C | PRO | G | 59 | 8.801 | 58.612 | 199.021 | 1.00 | 31.56 | C |
| ATOM | 2187 | O | PRO | G | 59 | 9.623 | 59.407 | 199.474 | 1.00 | 31.11 | O |
| ATOM | 2188 | N | ILE | G | 60 | 7.661 | 58.308 | 199.639 | 1.00 | 32.04 | N |
| ATOM | 2189 | CA | ILE | G | 60 | 7.366 | 58.811 | 200.970 | 1.00 | 32.85 | C |
| ATOM | 2191 | CB | ILE | G | 60 | 5.966 | 58.349 | 201.478 | 1.00 | 34.99 | C |
| ATOM | 2193 | CG1 | ILE | G | 60 | 4.851 | 58.989 | 200.646 | 1.00 | 36.38 | C |
| ATOM | 2196 | CD1 | ILE | G | 60 | 3.495 | 58.361 | 200.852 | 1.00 | 36.97 | C |
| ATOM | 2200 | CG2 | ILE | G | 60 | 5.771 | 58.700 | 202.951 | 1.00 | 34.30 | C |
| ATOM | 2204 | C | ILE | G | 60 | 8.486 | 58.295 | 201.875 | 1.00 | 32.54 | C |
| ATOM | 2205 | O | ILE | G | 60 | 8.860 | 57.116 | 201.821 | 1.00 | 30.77 | O |
| ATOM | 2207 | N | GLN | G | 61 | 9.039 | 59.192 | 202.683 | 1.00 | 32.33 | N |
| ATOM | 2208 | CA | GLN | G | 61 | 10.200 | 58.874 | 203.502 | 1.00 | 33.23 | C |
| ATOM | 2210 | CB | GLN | G | 61 | 10.947 | 60.155 | 203.876 | 1.00 | 33.19 | C |
| ATOM | 2213 | CG | GLN | G | 61 | 11.450 | 60.920 | 202.664 | 1.00 | 32.42 | C |
| ATOM | 2216 | CD | GLN | G | 61 | 12.626 | 60.243 | 202.013 | 1.00 | 31.12 | C |
| ATOM | 2217 | OE1 | GLN | G | 61 | 13.752 | 60.317 | 202.521 | 1.00 | 30.29 | O |
| ATOM | 2218 | NE2 | GLN | G | 61 | 12.384 | 59.575 | 200.884 | 1.00 | 30.02 | N |
| ATOM | 2221 | C | GLN | G | 61 | 9.751 | 58.139 | 204.750 | 1.00 | 32.25 | C |
| ATOM | 2222 | O | GLN | G | 61 | 8.879 | 58.615 | 205.483 | 1.00 | 33.47 | O |
| ATOM | 2224 | N | ARG | G | 62 | 10.319 | 56.966 | 204.979 | 1.00 | 34.65 | N |
| ATOM | 2225 | CA | ARG | G | 62 | 9.993 | 56.216 | 206.175 | 1.00 | 35.57 | C |
| ATOM | 2227 | CB | ARG | G | 62 | 8.710 | 55.403 | 206.004 | 1.00 | 39.53 | C |
| ATOM | 2230 | CG | ARG | G | 62 | 8.771 | 54.245 | 205.048 | 1.00 | 38.60 | C |
| ATOM | 2233 | CD | ARG | G | 62 | 8.120 | 52.989 | 205.635 | 1.00 | 41.73 | C |
| ATOM | 2236 | NE | ARG | G | 62 | 9.075 | 52.155 | 206.364 | 1.00 | 41.04 | N |
| ATOM | 2238 | CZ | ARG | G | 62 | 8.858 | 50.905 | 206.789 | 1.00 | 41.20 | C |
| ATOM | 2239 | NH1 | ARG | G | 62 | 7.705 | 50.291 | 206.590 | 1.00 | 44.67 | N |
| ATOM | 2242 | NH2 | ARG | G | 62 | 9.823 | 50.250 | 207.427 | 1.00 | 39.78 | N |
| ATOM | 2245 | C | ARG | G | 62 | 11.128 | 55.336 | 206.664 | 1.00 | 32.27 | C |
| ATOM | 2246 | O | ARG | G | 62 | 11.915 | 54.796 | 205.878 | 1.00 | 29.33 | O |
| ATOM | 2248 | N | LEU | G | 63 | 11.196 | 55.218 | 207.984 | 1.00 | 33.39 | N |
| ATOM | 2249 | CA | LEU | G | 63 | 12.178 | 54.374 | 208.641 | 1.00 | 32.48 | C |
| ATOM | 2251 | CB | LEU | G | 63 | 13.462 | 55.142 | 208.964 | 1.00 | 36.37 | C |
| ATOM | 2254 | CG | LEU | G | 63 | 14.504 | 54.229 | 209.640 | 1.00 | 38.06 | C |
| ATOM | 2256 | CD1 | LEU | G | 63 | 15.826 | 54.278 | 208.931 | 1.00 | 42.17 | C |
| ATOM | 2260 | CD2 | LEU | G | 63 | 14.641 | 54.533 | 211.126 | 1.00 | 39.69 | C |
| ATOM | 2264 | C | LEU | G | 63 | 11.599 | 53.802 | 209.919 | 1.00 | 31.87 | C |
| ATOM | 2265 | O | LEU | G | 63 | 10.984 | 54.515 | 210.712 | 1.00 | 29.73 | O |
| ATOM | 2267 | N | ARG | G | 64 | 11.829 | 52.509 | 210.106 | 1.00 | 33.15 | N |
| ATOM | 2268 | CA | ARG | G | 64 | 11.467 | 51.803 | 211.318 | 1.00 | 32.44 | C |
| ATOM | 2270 | CB | ARG | G | 64 | 10.428 | 50.737 | 210.979 | 1.00 | 32.19 | C |
| ATOM | 2273 | CG | ARG | G | 64 | 9.954 | 49.883 | 212.130 | 1.00 | 34.41 | C |
| ATOM | 2276 | CD | ARG | G | 64 | 9.093 | 48.766 | 211.573 | 1.00 | 36.89 | C |
| ATOM | 2279 | NE | ARG | G | 64 | 8.650 | 47.863 | 212.613 | 1.00 | 42.65 | N |
| ATOM | 2281 | CZ | ARG | G | 64 | 8.894 | 46.557 | 212.673 | 1.00 | 40.15 | C |
| ATOM | 2282 | NH1 | ARG | G | 64 | 9.559 | 45.906 | 211.714 | 1.00 | 40.83 | N |
| ATOM | 2285 | NH2 | ARG | G | 64 | 8.425 | 45.885 | 213.709 | 1.00 | 41.56 | N |
| ATOM | 2288 | C | ARG | G | 64 | 12.742 | 51.172 | 211.871 | 1.00 | 30.62 | C |
| ATOM | 2289 | O | ARG | G | 64 | 13.509 | 50.549 | 211.131 | 1.00 | 30.23 | O |
| ATOM | 2291 | N | GLY | G | 65 | 12.968 | 51.355 | 213.167 | 1.00 | 27.63 | N |
| ATOM | 2292 | CA | GLY | G | 65 | 14.164 | 50.850 | 213.822 | 1.00 | 31.87 | C |
| ATOM | 2295 | C | GLY | G | 65 | 13.849 | 50.205 | 215.157 | 1.00 | 31.53 | C |
| ATOM | 2296 | O | GLY | G | 65 | 12.975 | 50.674 | 215.896 | 1.00 | 32.78 | O |
| ATOM | 2298 | N | ALA | G | 66 | 14.558 | 49.123 | 215.463 | 1.00 | 31.27 | N |
| ATOM | 2299 | CA | ALA | G | 66 | 14.474 | 48.496 | 216.775 | 1.00 | 31.67 | C |
| ATOM | 2301 | CB | ALA | G | 66 | 14.890 | 47.037 | 216.698 | 1.00 | 33.23 | C |
| ATOM | 2305 | C | ALA | G | 66 | 15.379 | 49.254 | 217.742 | 1.00 | 34.13 | C |
| ATOM | 2306 | O | ALA | G | 66 | 16.432 | 49.758 | 217.343 | 1.00 | 31.04 | O |
| ATOM | 2308 | N | VAL | G | 67 | 14.958 | 49.330 | 219.005 | 1.00 | 34.89 | N |
| ATOM | 2309 | CA | VAL | G | 67 | 15.756 | 49.935 | 220.075 | 1.00 | 36.20 | C |
| ATOM | 2311 | CB | VAL | G | 67 | 15.114 | 51.260 | 220.587 | 1.00 | 37.26 | C |
| ATOM | 2313 | CG1 | VAL | G | 67 | 16.028 | 51.952 | 221.588 | 1.00 | 39.66 | C |
| ATOM | 2317 | CG2 | VAL | G | 67 | 14.824 | 52.203 | 219.424 | 1.00 | 37.92 | C |
| ATOM | 2321 | C | VAL | G | 67 | 15.892 | 48.902 | 221.212 | 1.00 | 34.56 | C |
| ATOM | 2322 | O | VAL | G | 67 | 14.917 | 48.594 | 221.902 | 1.00 | 31.76 | O |
| ATOM | 2324 | N | THR | G | 68 | 17.090 | 48.345 | 221.377 | 1.00 | 36.57 | N |
| ATOM | 2325 | CA | THR | G | 68 | 17.341 | 47.336 | 222.421 | 1.00 | 37.78 | C |
| ATOM | 2327 | CB | THR | G | 68 | 17.781 | 45.977 | 221.812 | 1.00 | 38.26 | C |
| ATOM | 2329 | OG1 | THR | G | 68 | 19.046 | 46.125 | 221.158 | 1.00 | 38.23 | O |
| ATOM | 2331 | CG2 | THR | G | 68 | 16.752 | 45.470 | 220.807 | 1.00 | 38.50 | C |
| ATOM | 2335 | C | THR | G | 68 | 18.393 | 47.746 | 223.453 | 1.00 | 39.29 | C |
| ATOM | 2336 | O | THR | G | 68 | 18.407 | 47.209 | 224.563 | 1.00 | 40.04 | O |
| ATOM | 2338 | N | ARG | G | 69 | 19.278 | 48.672 | 223.088 | 1.00 | 38.78 | N |
| ATOM | 2339 | CA | ARG | G | 69 | 20.366 | 49.080 | 223.968 | 1.00 | 39.85 | C |
| ATOM | 2341 | CB | ARG | G | 69 | 21.605 | 48.209 | 223.715 | 1.00 | 41.06 | C |
| ATOM | 2344 | CG | ARG | G | 69 | 22.199 | 48.336 | 222.314 | 1.00 | 41.88 | C |
| ATOM | 2347 | CD | ARG | G | 69 | 23.584 | 47.712 | 222.244 | 1.00 | 44.11 | C |
| ATOM | 2350 | NE | ARG | G | 69 | 24.276 | 48.048 | 221.000 | 1.00 | 44.78 | N |
| ATOM | 2352 | CZ | ARG | G | 69 | 24.065 | 47.459 | 219.824 | 1.00 | 47.01 | C |
| ATOM | 2353 | NH1 | ARG | G | 69 | 23.169 | 46.481 | 219.692 | 1.00 | 46.96 | N |
| ATOM | 2356 | NH2 | ARG | G | 69 | 24.761 | 47.853 | 218.763 | 1.00 | 47.96 | N |
| ATOM | 2359 | C | ARG | G | 69 | 20.709 | 50.556 | 223.786 | 1.00 | 37.60 | C |
| ATOM | 2360 | O | ARG | G | 69 | 20.137 | 51.236 | 222.936 | 1.00 | 30.53 | O |
| ATOM | 2362 | N | CYS | G | 70 | 21.642 | 51.034 | 224.605 | 1.00 | 37.33 | N |
| ATOM | 2363 | CA | CYS | G | 70 | 22.145 | 52.401 | 224.542 | 1.00 | 39.49 | C |
| ATOM | 2365 | CB | CYS | G | 70 | 21.894 | 53.104 | 225.888 | 1.00 | 42.65 | C |
| ATOM | 2368 | SG | CYS | G | 70 | 22.723 | 54.713 | 226.170 | 1.00 | 45.86 | S |
| ATOM | 2370 | C | CYS | G | 70 | 23.638 | 52.334 | 224.224 | 1.00 | 39.98 | C |
| ATOM | 2371 | O | CYS | G | 70 | 24.341 | 51.451 | 224.726 | 1.00 | 37.75 | O |
| ATOM | 2373 | N | GLU | G | 71 | 24.115 | 53.254 | 223.388 | 1.00 | 41.30 | N |
| ATOM | 2374 | CA | GLU | G | 71 | 25.536 | 53.318 | 223.035 | 1.00 | 42.86 | C |
| ATOM | 2376 | CB | GLU | G | 71 | 25.799 | 52.553 | 221.732 | 1.00 | 43.82 | C |
| ATOM | 2379 | CG | GLU | G | 71 | 27.233 | 52.043 | 221.601 | 1.00 | 46.31 | C |
| ATOM | 2382 | CD | GLU | G | 71 | 27.545 | 51.473 | 220.225 | 1.00 | 47.91 | C |
| ATOM | 2383 | OE1 | GLU | G | 71 | 26.645 | 50.883 | 219.590 | 1.00 | 48.78 | O |
| ATOM | 2384 | OE2 | GLU | G | 71 | 28.707 | 51.607 | 219.784 | 1.00 | 53.02 | O |
| ATOM | 2385 | C | GLU | G | 71 | 25.988 | 54.776 | 222.901 | 1.00 | 43.03 | C |
| ATOM | 2386 | O | GLU | G | 71 | 25.346 | 55.563 | 222.201 | 1.00 | 43.33 | O |
| ATOM | 2388 | N | ASP | G | 72 | 27.084 | 55.120 | 223.579 | 1.00 | 40.96 | N |
| ATOM | 2389 | CA | ASP | G | 72 | 27.640 | 56.485 | 223.589 | 1.00 | 40.54 | C |
| ATOM | 2391 | CB | ASP | G | 72 | 28.257 | 56.835 | 222.221 | 1.00 | 43.44 | C |
| ATOM | 2394 | CG | ASP | G | 72 | 29.514 | 56.029 | 221.913 | 1.00 | 46.62 | C |
| ATOM | 2395 | OD1 | ASP | G | 72 | 30.129 | 55.471 | 222.847 | 1.00 | 46.67 | O |
| ATOM | 2396 | OD2 | ASP | G | 72 | 29.893 | 55.963 | 220.723 | 1.00 | 49.36 | O |
| ATOM | 2397 | C | ASP | G | 72 | 26.627 | 57.560 | 224.019 | 1.00 | 38.62 | C |
| ATOM | 2398 | O | ASP | G | 72 | 26.569 | 58.648 | 223.438 | 1.00 | 39.45 | O |
| ATOM | 2400 | N | GLY | G | 73 | 25.837 | 57.246 | 225.044 | 1.00 | 35.70 | N |
| ATOM | 2401 | CA | GLY | G | 73 | 24.854 | 58.186 | 225.588 | 1.00 | 34.85 | C |
| ATOM | 2404 | C | GLY | G | 73 | 23.612 | 58.368 | 224.734 | 1.00 | 31.04 | C |
| ATOM | 2405 | O | GLY | G | 73 | 22.847 | 59.312 | 224.941 | 1.00 | 28.92 | O |
| ATOM | 2407 | N | GLN | G | 74 | 23.412 | 57.466 | 223.776 | 1.00 | 30.10 | N |
| ATOM | 2408 | CA | GLN | G | 74 | 22.260 | 57.517 | 222.876 | 1.00 | 32.77 | C |
| ATOM | 2410 | CB | GLN | G | 74 | 22.688 | 57.974 | 221.476 | 1.00 | 33.58 | C |
| ATOM | 2413 | CG | GLN | G | 74 | 23.211 | 59.408 | 221.414 | 1.00 | 34.38 | C |
| ATOM | 2416 | CD | GLN | G | 74 | 23.616 | 59.852 | 220.010 | 1.00 | 35.12 | C |
| ATOM | 2417 | OE1 | GLN | G | 74 | 24.187 | 60.930 | 219.833 | 1.00 | 35.60 | O |
| ATOM | 2418 | NE2 | GLN | G | 74 | 23.324 | 59.027 | 219.011 | 1.00 | 34.58 | N |
| ATOM | 2421 | C | GLN | G | 74 | 21.596 | 56.149 | 222.776 | 1.00 | 32.07 | C |
| ATOM | 2422 | O | GLN | G | 74 | 22.268 | 55.115 | 222.836 | 1.00 | 31.12 | O |
| ATOM | 2424 | N | LEU | G | 75 | 20.275 | 56.146 | 222.640 | 1.00 | 30.91 | N |
| ATOM | 2425 | CA | LEU | G | 75 | 19.551 | 54.924 | 222.313 | 1.00 | 32.20 | C |
| ATOM | 2427 | CB | LEU | G | 75 | 18.038 | 55.165 | 222.307 | 1.00 | 33.14 | C |
| ATOM | 2430 | CG | LEU | G | 75 | 17.418 | 55.612 | 223.637 | 1.00 | 33.79 | C |
| ATOM | 2432 | CD1 | LEU | G | 75 | 15.935 | 55.858 | 223.462 | 1.00 | 35.55 | C |
| ATOM | 2436 | CD2 | LEU | G | 75 | 17.671 | 54.580 | 224.715 | 1.00 | 36.74 | C |
| ATOM | 2440 | C | LEU | G | 75 | 20.024 | 54.458 | 220.939 | 1.00 | 30.90 | C |
| ATOM | 2441 | O | LEU | G | 75 | 20.086 | 55.254 | 219.999 | 1.00 | 30.31 | O |
| ATOM | 2443 | N | PHE | G | 76 | 20.381 | 53.181 | 220.828 | 1.00 | 33.35 | N |
| ATOM | 2444 | CA | PHE | G | 76 | 20.899 | 52.650 | 219.571 | 1.00 | 32.90 | C |
| ATOM | 2446 | CB | PHE | G | 76 | 21.833 | 51.458 | 219.801 | 1.00 | 38.12 | C |
| ATOM | 2449 | CG | PHE | G | 76 | 22.585 | 51.045 | 218.567 | 1.00 | 37.48 | C |
| ATOM | 2450 | CD1 | PHE | G | 76 | 23.706 | 51.753 | 218.156 | 1.00 | 39.99 | C |
| ATOM | 2452 | CE1 | PHE | G | 76 | 24.398 | 51.390 | 217.003 | 1.00 | 40.19 | C |
| ATOM | 2454 | CZ | PHE | G | 76 | 23.961 | 50.316 | 216.244 | 1.00 | 40.18 | C |
| ATOM | 2456 | CE2 | PHE | G | 76 | 22.833 | 49.608 | 216.638 | 1.00 | 40.03 | C |
| ATOM | 2458 | CD2 | PHE | G | 76 | 22.150 | 49.977 | 217.794 | 1.00 | 39.48 | C |
| ATOM | 2460 | C | PHE | G | 76 | 19.745 | 52.257 | 218.644 | 1.00 | 33.13 | C |
| ATOM | 2461 | O | PHE | G | 76 | 18.894 | 51.443 | 219.005 | 1.00 | 33.12 | O |
| ATOM | 2463 | N | ILE | G | 77 | 19.722 | 52.851 | 217.456 | 1.00 | 32.32 | N |
| ATOM | 2464 | CA | ILE | G | 77 | 18.689 | 52.563 | 216.457 | 1.00 | 32.99 | C |
| ATOM | 2466 | CB | ILE | G | 77 | 18.334 | 53.810 | 215.625 | 1.00 | 32.56 | C |
| ATOM | 2468 | CG1 | ILE | G | 77 | 17.933 | 54.973 | 216.535 | 1.00 | 34.51 | C |
| ATOM | 2471 | CD1 | ILE | G | 77 | 18.078 | 56.330 | 215.870 | 1.00 | 36.53 | C |
| ATOM | 2475 | CG2 | ILE | G | 77 | 17.210 | 53.488 | 214.618 | 1.00 | 34.75 | C |
| ATOM | 2479 | C | ILE | G | 77 | 19.180 | 51.477 | 215.507 | 1.00 | 32.47 | C |
| ATOM | 2480 | O | ILE | G | 77 | 20.129 | 51.687 | 214.747 | 1.00 | 32.81 | O |
| ATOM | 2482 | N | SER | G | 78 | 18.532 | 50.318 | 215.560 | 1.00 | 33.80 | N |
| ATOM | 2483 | CA | SER | G | 78 | 18.846 | 49.210 | 214.669 | 1.00 | 35.35 | C |
| ATOM | 2485 | CB | SER | G | 78 | 18.867 | 47.895 | 215.445 | 1.00 | 39.07 | C |
| ATOM | 2488 | OG | SER | G | 78 | 19.058 | 46.799 | 214.573 | 1.00 | 44.44 | O |
| ATOM | 2490 | C | SER | G | 78 | 17.814 | 49.163 | 213.537 | 1.00 | 35.46 | C |
| ATOM | 2491 | O | SER | G | 78 | 16.692 | 48.679 | 213.719 | 1.00 | 33.39 | O |
| ATOM | 2493 | N | SER | G | 79 | 18.210 | 49.674 | 212.374 | 1.00 | 33.37 | N |
| ATOM | 2494 | CA | SER | G | 79 | 17.316 | 49.801 | 211.232 | 1.00 | 35.99 | C |
| ATOM | 2496 | CB | SER | G | 79 | 17.925 | 50.747 | 210.194 | 1.00 | 38.04 | C |
| ATOM | 2499 | OG | SER | G | 79 | 17.029 | 50.975 | 209.122 | 1.00 | 42.21 | O |
| ATOM | 2501 | C | SER | G | 79 | 17.058 | 48.428 | 210.613 | 1.00 | 36.83 | C |
| ATOM | 2502 | O | SER | G | 79 | 17.993 | 47.670 | 210.361 | 1.00 | 36.03 | O |
| ATOM | 2504 | N | TYR | G | 80 | 15.788 | 48.106 | 210.385 | 1.00 | 34.22 | N |
| ATOM | 2505 | CA | TYR | G | 80 | 15.421 | 46.839 | 209.754 | 1.00 | 34.82 | C |
| ATOM | 2507 | CB | TYR | G | 80 | 13.913 | 46.613 | 209.844 | 1.00 | 34.39 | C |
| ATOM | 2510 | CG | TYR | G | 80 | 13.442 | 46.302 | 211.246 | 1.00 | 33.00 | C |
| ATOM | 2511 | CD1 | TYR | G | 80 | 13.630 | 45.039 | 211.793 | 1.00 | 33.92 | C |
| ATOM | 2513 | CE1 | TYR | G | 80 | 13.201 | 44.742 | 213.077 | 1.00 | 33.97 | C |
| ATOM | 2515 | CZ | TYR | G | 80 | 12.583 | 45.715 | 213.835 | 1.00 | 34.47 | C |
| ATOM | 2516 | OH | TYR | G | 80 | 12.165 | 45.407 | 215.107 | 1.00 | 34.94 | O |
| ATOM | 2518 | CE2 | TYR | G | 80 | 12.381 | 46.980 | 213.317 | 1.00 | 33.88 | C |
| ATOM | 2520 | CD2 | TYR | G | 80 | 12.811 | 47.266 | 212.023 | 1.00 | 33.40 | C |
| ATOM | 2522 | C | TYR | G | 80 | 15.877 | 46.789 | 208.295 | 1.00 | 34.25 | C |
| ATOM | 2523 | O | TYR | G | 80 | 15.924 | 47.816 | 207.619 | 1.00 | 36.18 | O |
| ATOM | 2525 | N | LYS | G | 81 | 16.237 | 45.592 | 207.838 | 1.00 | 35.25 | N |
| ATOM | 2526 | CA | LYS | G | 81 | 16.579 | 45.343 | 206.435 | 1.00 | 35.81 | C |
| ATOM | 2528 | CB | LYS | G | 81 | 17.732 | 44.409 | 206.304 | 0.00 | 30.00 | C |
| ATOM | 2531 | CG | LYS | G | 81 | 18.174 | 44.256 | 204.849 | 0.00 | 30.00 | C |
| ATOM | 2534 | CD | LYS | G | 81 | 19.317 | 43.248 | 204.742 | 0.00 | 30.00 | C |
| ATOM | 2537 | CE | LYS | G | 81 | 19.771 | 43.107 | 203.290 | 0.00 | 30.00 | C |
| ATOM | 2540 | NZ | LYS | G | 81 | 20.882 | 42.107 | 203.181 | 0.00 | 30.00 | N |
| ATOM | 2544 | C | LYS | G | 81 | 15.316 | 44.914 | 205.681 | 1.00 | 35.07 | C |
| ATOM | 2545 | O | LYS | G | 81 | 14.890 | 43.759 | 205.776 | 1.00 | 37.10 | O |
| ATOM | 2547 | N | ASN | G | 82 | 14.721 | 45.848 | 204.942 | 1.00 | 32.01 | N |
| ATOM | 2548 | CA | ASN | G | 82 | 13.441 | 45.612 | 204.275 | 1.00 | 31.17 | C |
| ATOM | 2550 | CB | ASN | G | 82 | 12.297 | 45.717 | 205.306 | 1.00 | 34.26 | C |
| ATOM | 2553 | CG | ASN | G | 82 | 10.931 | 45.338 | 204.728 | 1.00 | 35.43 | C |
| ATOM | 2554 | OD1 | ASN | G | 82 | 10.149 | 46.207 | 204.351 | 1.00 | 35.57 | O |
| ATOM | 2555 | ND2 | ASN | G | 82 | 10.645 | 44.042 | 204.664 | 1.00 | 33.34 | N |
| ATOM | 2558 | C | ASN | G | 82 | 13.224 | 46.606 | 203.122 | 1.00 | 31.23 | C |
| ATOM | 2559 | O | ASN | G | 82 | 13.679 | 47.752 | 203.179 | 1.00 | 28.31 | O |
| ATOM | 2561 | N | GLU | G | 83 | 12.511 | 46.151 | 202.091 | 1.00 | 28.49 | N |
| ATOM | 2562 | CA | GLU | G | 83 | 12.200 | 46.954 | 200.896 | 1.00 | 29.72 | C |
| ATOM | 2564 | CB | GLU | G | 83 | 11.380 | 46.112 | 199.896 | 1.00 | 29.27 | C |
| ATOM | 2567 | CG | GLU | G | 83 | 10.005 | 45.669 | 200.428 | 1.00 | 27.52 | C |
| ATOM | 2570 | CD | GLU | G | 83 | 9.242 | 44.789 | 199.452 | 1.00 | 29.63 | C |
| ATOM | 2571 | OE1 | GLU | G | 83 | 9.424 | 44.961 | 198.234 | 1.00 | 28.62 | O |
| ATOM | 2572 | OE2 | GLU | G | 83 | 8.460 | 43.923 | 199.906 | 1.00 | 26.96 | O |
| ATOM | 2573 | C | GLU | G | 83 | 11.458 | 48.267 | 201.182 | 1.00 | 31.17 | C |
| ATOM | 2574 | O | GLU | G | 83 | 11.569 | 49.222 | 200.410 | 1.00 | 31.63 | O |
| ATOM | 2576 | N | TYR | G | 84 | 10.696 | 48.315 | 202.274 | 1.00 | 29.16 | N |
| ATOM | 2577 | CA | TYR | G | 84 | 9.898 | 49.500 | 202.601 | 1.00 | 29.45 | C |
| ATOM | 2579 | CB | TYR | G | 84 | 8.702 | 49.109 | 203.477 | 1.00 | 30.73 | C |
| ATOM | 2582 | CG | TYR | G | 84 | 7.701 | 48.184 | 202.797 | 1.00 | 28.86 | C |
| ATOM | 2583 | CD1 | TYR | G | 84 | 7.252 | 48.437 | 201.502 | 1.00 | 30.14 | C |
| ATOM | 2585 | CE1 | TYR | G | 84 | 6.330 | 47.605 | 200.879 | 1.00 | 29.91 | C |
| ATOM | 2587 | CZ | TYR | G | 84 | 5.831 | 46.507 | 201.556 | 1.00 | 29.47 | C |
| ATOM | 2588 | OH | TYR | G | 84 | 4.919 | 45.678 | 200.932 | 1.00 | 27.45 | O |
| ATOM | 2590 | CE2 | TYR | G | 84 | 6.255 | 46.233 | 202.844 | 1.00 | 30.11 | C |
| ATOM | 2592 | CD2 | TYR | G | 84 | 7.191 | 47.069 | 203.460 | 1.00 | 31.27 | C |
| ATOM | 2594 | C | TYR | G | 84 | 10.679 | 50.637 | 203.266 | 1.00 | 28.70 | C |
| ATOM | 2595 | O | TYR | G | 84 | 10.154 | 51.743 | 203.415 | 1.00 | 30.02 | O |
| ATOM | 2597 | N | GLN | G | 85 | 11.924 | 50.375 | 203.660 | 1.00 | 30.01 | N |
| ATOM | 2598 | CA | GLN | G | 85 | 12.772 | 51.403 | 204.259 | 1.00 | 28.47 | C |
| ATOM | 2600 | CB | GLN | G | 85 | 13.966 | 50.753 | 204.968 | 1.00 | 32.41 | C |
| ATOM | 2603 | CG | GLN | G | 85 | 13.595 | 49.755 | 206.063 | 1.00 | 30.80 | C |
| ATOM | 2606 | CD | GLN | G | 85 | 13.358 | 50.406 | 207.409 | 1.00 | 32.52 | C |
| ATOM | 2607 | OE1 | GLN | G | 85 | 12.573 | 51.339 | 207.524 | 1.00 | 33.54 | O |
| ATOM | 2608 | NE2 | GLN | G | 85 | 14.047 | 49.920 | 208.436 | 1.00 | 35.09 | N |
| ATOM | 2611 | C | GLN | G | 85 | 13.263 | 52.369 | 203.169 | 1.00 | 29.95 | C |
| ATOM | 2612 | O | GLN | G | 85 | 13.874 | 51.940 | 202.189 | 1.00 | 26.27 | O |
| ATOM | 2614 | N | THR | G | 86 | 12.986 | 53.662 | 203.332 | 1.00 | 28.12 | N |
| ATOM | 2615 | CA | THR | G | 86 | 13.379 | 54.674 | 202.339 | 1.00 | 27.66 | C |
| ATOM | 2617 | CB | THR | G | 86 | 12.144 | 55.267 | 201.620 | 1.00 | 26.80 | C |
| ATOM | 2619 | OG1 | THR | G | 86 | 11.261 | 55.852 | 202.578 | 1.00 | 29.71 | O |
| ATOM | 2621 | CG2 | THR | G | 86 | 11.389 | 54.185 | 200.835 | 1.00 | 27.57 | C |
| ATOM | 2625 | C | THR | G | 86 | 14.220 | 55.813 | 202.931 | 1.00 | 31.54 | C |
| ATOM | 2626 | O | THR | G | 86 | 14.463 | 56.812 | 202.256 | 1.00 | 29.35 | O |
| ATOM | 2628 | N | MET | G | 87 | 14.654 | 55.662 | 204.186 | 1.00 | 32.94 | N |
| ATOM | 2629 | CA | MET | G | 87 | 15.596 | 56.589 | 204.820 | 1.00 | 33.98 | C |
| ATOM | 2631 | CB | MET | G | 87 | 14.924 | 57.383 | 205.938 | 1.00 | 34.16 | C |
| ATOM | 2634 | CG | MET | G | 87 | 14.033 | 58.509 | 205.445 | 1.00 | 37.77 | C |
| ATOM | 2637 | SD | MET | G | 87 | 13.464 | 59.574 | 206.794 | 1.00 | 42.70 | S |
| ATOM | 2638 | CE | MET | G | 87 | 12.218 | 58.543 | 207.441 | 1.00 | 32.33 | C |
| ATOM | 2642 | C | MET | G | 87 | 16.772 | 55.813 | 205.395 | 1.00 | 34.94 | C |
| ATOM | 2643 | O | MET | G | 87 | 16.665 | 54.611 | 205.663 | 1.00 | 35.43 | O |
| ATOM | 2645 | N | GLU | G | 88 | 17.884 | 56.521 | 205.585 | 1.00 | 32.93 | N |
| ATOM | 2646 | CA | GLU | G | 88 | 19.118 | 55.946 | 206.108 | 1.00 | 35.69 | C |
| ATOM | 2648 | CB | GLU | G | 88 | 20.309 | 56.425 | 205.272 | 1.00 | 41.28 | C |
| ATOM | 2651 | CG | GLU | G | 88 | 20.350 | 55.844 | 203.859 | 1.00 | 48.44 | C |
| ATOM | 2654 | CD | GLU | G | 88 | 21.501 | 54.877 | 203.650 | 1.00 | 54.05 | C |
| ATOM | 2655 | OE1 | GLU | G | 88 | 22.662 | 55.342 | 203.626 | 1.00 | 56.38 | O |
| ATOM | 2656 | OE2 | GLU | G | 88 | 21.246 | 53.660 | 203.500 | 1.00 | 57.43 | O |
| ATOM | 2657 | C | GLU | G | 88 | 19.338 | 56.338 | 207.565 | 1.00 | 35.58 | C |
| ATOM | 2658 | O | GLU | G | 88 | 18.933 | 57.420 | 207.999 | 1.00 | 31.94 | O |
| ATOM | 2660 | N | VAL | G | 89 | 19.967 | 55.436 | 208.314 | 1.00 | 34.34 | N |
| ATOM | 2661 | CA | VAL | G | 89 | 20.490 | 55.744 | 209.637 | 1.00 | 35.48 | C |
| ATOM | 2663 | CB | VAL | G | 89 | 20.088 | 54.681 | 210.697 | 1.00 | 36.35 | C |
| ATOM | 2665 | CG1 | VAL | G | 89 | 20.855 | 54.884 | 212.008 | 1.00 | 36.55 | C |
| ATOM | 2669 | CG2 | VAL | G | 89 | 18.592 | 54.719 | 210.943 | 1.00 | 38.46 | C |
| ATOM | 2673 | C | VAL | G | 89 | 22.003 | 55.799 | 209.499 | 1.00 | 36.90 | C |
| ATOM | 2674 | O | VAL | G | 89 | 22.610 | 54.860 | 208.982 | 1.00 | 36.19 | O |
| ATOM | 2676 | N | GLN | G | 90 | 22.593 | 56.908 | 209.939 | 1.00 | 37.51 | N |
| ATOM | 2677 | CA | GLN | G | 90 | 24.036 | 57.118 | 209.898 | 1.00 | 38.77 | C |
| ATOM | 2679 | CB | GLN | G | 90 | 24.397 | 58.098 | 208.778 | 1.00 | 40.08 | C |
| ATOM | 2682 | CG | GLN | G | 90 | 25.895 | 58.204 | 208.495 | 1.00 | 44.38 | C |
| ATOM | 2685 | CD | GLN | G | 90 | 26.304 | 59.535 | 207.874 | 1.00 | 46.83 | C |
| ATOM | 2686 | OE1 | GLN | G | 90 | 27.440 | 59.990 | 208.053 | 1.00 | 52.00 | O |
| ATOM | 2687 | NE2 | GLN | G | 90 | 25.385 | 60.165 | 207.145 | 1.00 | 48.23 | N |
| ATOM | 2690 | C | GLN | G | 90 | 24.470 | 57.687 | 211.247 | 1.00 | 35.50 | C |
| ATOM | 2691 | O | GLN | G | 90 | 23.825 | 58.595 | 211.771 | 1.00 | 33.92 | O |
| ATOM | 2693 | N | ASN | G | 91 | 25.553 | 57.152 | 211.807 | 1.00 | 37.28 | N |
| ATOM | 2694 | CA | ASN | G | 91 | 26.051 | 57.579 | 213.117 | 1.00 | 36.91 | C |
| ATOM | 2696 | CB | ASN | G | 91 | 26.722 | 58.955 | 213.015 | 1.00 | 40.43 | C |
| ATOM | 2699 | CG | ASN | G | 91 | 28.016 | 58.919 | 212.215 | 1.00 | 44.77 | C |
| ATOM | 2700 | OD1 | ASN | G | 91 | 28.790 | 57.966 | 212.302 | 1.00 | 46.89 | O |
| ATOM | 2701 | ND2 | ASN | G | 91 | 28.261 | 59.970 | 211.440 | 1.00 | 48.21 | N |
| ATOM | 2704 | C | ASN | G | 91 | 24.945 | 57.585 | 214.176 | 1.00 | 35.82 | C |
| ATOM | 2705 | O | ASN | G | 91 | 24.845 | 58.508 | 214.991 | 1.00 | 34.98 | O |
| ATOM | 2707 | N | ASN | G | 92 | 24.120 | 56.541 | 214.140 | 1.00 | 33.65 | N |
| ATOM | 2708 | CA | ASN | G | 92 | 23.006 | 56.357 | 215.068 | 1.00 | 35.02 | C |
| ATOM | 2710 | CB | ASN | G | 92 | 23.532 | 56.101 | 216.490 | 1.00 | 37.01 | C |
| ATOM | 2713 | CG | ASN | G | 92 | 22.491 | 55.462 | 217.390 | 1.00 | 36.62 | C |
| ATOM | 2714 | OD1 | ASN | G | 92 | 21.617 | 54.732 | 216.922 | 1.00 | 35.62 | O |
| ATOM | 2715 | ND2 | ASN | G | 92 | 22.572 | 55.742 | 218.689 | 1.00 | 36.66 | N |
| ATOM | 2718 | C | ASN | G | 92 | 21.992 | 57.512 | 215.050 | 1.00 | 33.94 | C |
| ATOM | 2719 | O | ASN | G | 92 | 21.452 | 57.903 | 216.087 | 1.00 | 33.96 | O |
| ATOM | 2721 | N | SER | G | 93 | 21.743 | 58.049 | 213.858 | 1.00 | 34.57 | N |
| ATOM | 2722 | CA | SER | G | 93 | 20.694 | 59.048 | 213.656 | 1.00 | 33.69 | C |
| ATOM | 2724 | CB | SER | G | 93 | 21.277 | 60.467 | 213.682 | 1.00 | 35.98 | C |
| ATOM | 2727 | OG | SER | G | 93 | 22.139 | 60.703 | 212.583 | 1.00 | 38.49 | O |
| ATOM | 2729 | C | SER | G | 93 | 19.971 | 58.787 | 212.340 | 1.00 | 33.03 | C |
| ATOM | 2730 | O | SER | G | 93 | 20.555 | 58.249 | 211.402 | 1.00 | 29.97 | O |
| ATOM | 2732 | N | VAL | G | 94 | 18.693 | 59.150 | 212.290 | 1.00 | 32.02 | N |
| ATOM | 2733 | CA | VAL | G | 94 | 17.908 | 59.079 | 211.065 | 1.00 | 32.18 | C |
| ATOM | 2735 | CB | VAL | G | 94 | 16.404 | 58.968 | 211.363 | 1.00 | 31.71 | C |
| ATOM | 2737 | CG1 | VAL | G | 94 | 15.608 | 58.881 | 210.075 | 1.00 | 33.15 | C |
| ATOM | 2741 | CG2 | VAL | G | 94 | 16.131 | 57.746 | 212.258 | 1.00 | 33.24 | C |
| ATOM | 2745 | C | VAL | G | 94 | 18.191 | 60.337 | 210.238 | 1.00 | 32.39 | C |
| ATOM | 2746 | O | VAL | G | 94 | 17.925 | 61.457 | 210.683 | 1.00 | 30.37 | O |
| ATOM | 2748 | N | VAL | G | 95 | 18.734 | 60.130 | 209.042 | 1.00 | 30.33 | N |
| ATOM | 2749 | CA | VAL | G | 95 | 19.131 | 61.218 | 208.151 | 1.00 | 30.86 | C |
| ATOM | 2751 | CB | VAL | G | 95 | 20.213 | 60.748 | 207.160 | 1.00 | 32.28 | C |
| ATOM | 2753 | CG1 | VAL | G | 95 | 20.644 | 61.891 | 206.248 | 1.00 | 32.89 | C |
| ATOM | 2757 | CG2 | VAL | G | 95 | 21.412 | 60.172 | 207.913 | 1.00 | 32.79 | C |
| ATOM | 2761 | C | VAL | G | 95 | 17.929 | 61.738 | 207.366 | 1.00 | 31.94 | C |
| ATOM | 2762 | O | VAL | G | 95 | 17.297 | 60.989 | 206.606 | 1.00 | 32.36 | O |
| ATOM | 2764 | N | ILE | G | 96 | 17.621 | 63.021 | 207.547 | 1.00 | 29.86 | N |
| ATOM | 2765 | CA | ILE | G | 96 | 16.508 | 63.653 | 206.836 | 1.00 | 31.13 | C |
| ATOM | 2767 | CB | ILE | G | 96 | 15.826 | 64.762 | 207.681 | 1.00 | 29.87 | C |
| ATOM | 2769 | CG1 | ILE | G | 96 | 15.391 | 64.232 | 209.059 | 1.00 | 31.88 | C |
| ATOM | 2772 | CD1 | ILE | G | 96 | 14.472 | 63.013 | 209.018 | 1.00 | 31.79 | C |
| ATOM | 2776 | CG2 | ILE | G | 96 | 14.637 | 65.366 | 206.927 | 1.00 | 29.46 | C |
| ATOM | 2780 | C | ILE | G | 96 | 17.026 | 64.236 | 205.519 | 1.00 | 30.74 | C |
| ATOM | 2781 | O | ILE | G | 96 | 17.856 | 65.148 | 205.511 | 1.00 | 28.33 | O |
| ATOM | 2783 | N | LYS | G | 97 | 16.547 | 63.683 | 204.410 | 1.00 | 35.40 | N |
| ATOM | 2784 | CA | LYS | G | 97 | 16.905 | 64.171 | 203.080 | 1.00 | 35.66 | C |
| ATOM | 2786 | CB | LYS | G | 97 | 17.234 | 63.002 | 202.140 | 1.00 | 42.38 | C |
| ATOM | 2789 | CG | LYS | G | 97 | 18.564 | 62.305 | 202.447 | 1.00 | 47.38 | C |
| ATOM | 2792 | CD | LYS | G | 97 | 19.782 | 63.213 | 202.209 | 1.00 | 47.37 | C |
| ATOM | 2795 | CE | LYS | G | 97 | 21.077 | 62.534 | 202.608 | 1.00 | 48.34 | C |
| ATOM | 2798 | NZ | LYS | G | 97 | 22.227 | 63.491 | 202.715 | 1.00 | 49.53 | N |
| ATOM | 2802 | C | LYS | G | 97 | 15.810 | 65.039 | 202.468 | 1.00 | 33.17 | C |
| ATOM | 2803 | O | LYS | G | 97 | 16.101 | 65.844 | 201.593 | 1.00 | 30.00 | O |
| ATOM | 2805 | N | CYS | G | 98 | 14.563 | 64.869 | 202.924 | 1.00 | 32.18 | N |
| ATOM | 2806 | CA | CYS | G | 98 | 13.425 | 65.596 | 202.370 | 1.00 | 31.83 | C |
| ATOM | 2808 | CB | CYS | G | 98 | 12.415 | 64.625 | 201.745 | 1.00 | 34.55 | C |
| ATOM | 2811 | SG | CYS | G | 98 | 13.092 | 63.548 | 200.463 | 1.00 | 34.35 | S |
| ATOM | 2813 | C | CYS | G | 98 | 12.714 | 66.436 | 203.433 | 1.00 | 32.93 | C |
| ATOM | 2814 | O | CYS | G | 98 | 12.455 | 65.960 | 204.545 | 1.00 | 31.21 | O |
| ATOM | 2816 | N | ASP | G | 99 | 12.390 | 67.676 | 203.072 | 1.00 | 29.60 | N |
| ATOM | 2817 | CA | ASP | G | 99 | 11.555 | 68.539 | 203.896 | 1.00 | 28.79 | C |
| ATOM | 2819 | CB | ASP | G | 99 | 11.327 | 69.906 | 203.235 | 1.00 | 29.04 | C |
| ATOM | 2822 | CG | ASP | G | 99 | 12.567 | 70.753 | 203.150 | 1.00 | 31.31 | C |
| ATOM | 2823 | OD1 | ASP | G | 99 | 13.649 | 70.326 | 203.612 | 1.00 | 31.26 | O |
| ATOM | 2824 | OD2 | ASP | G | 99 | 12.433 | 71.872 | 202.615 | 1.00 | 29.27 | O |
| ATOM | 2825 | C | ASP | G | 99 | 10.175 | 67.913 | 204.046 | 1.00 | 31.17 | C |
| ATOM | 2826 | O | ASP | G | 99 | 9.706 | 67.210 | 203.156 | 1.00 | 30.40 | O |
| ATOM | 2828 | N | GLY | G | 100 | 9.506 | 68.229 | 205.149 | 1.00 | 33.55 | N |
| ATOM | 2829 | CA | GLY | G | 100 | 8.118 | 67.840 | 205.330 | 1.00 | 31.26 | C |
| ATOM | 2832 | C | GLY | G | 100 | 7.728 | 67.807 | 206.788 | 1.00 | 31.48 | C |
| ATOM | 2833 | O | GLY | G | 100 | 8.570 | 67.992 | 207.688 | 1.00 | 29.36 | O |
| ATOM | 2835 | N | LEU | G | 101 | 6.439 | 67.595 | 207.021 | 1.00 | 27.92 | N |
| ATOM | 2836 | CA | LEU | G | 101 | 5.975 | 67.217 | 208.342 | 1.00 | 27.87 | C |
| ATOM | 2838 | CB | LEU | G | 101 | 4.511 | 67.589 | 208.559 | 1.00 | 28.54 | C |
| ATOM | 2841 | CG | LEU | G | 101 | 4.152 | 69.060 | 208.410 | 1.00 | 32.99 | C |
| ATOM | 2843 | CD1 | LEU | G | 101 | 2.679 | 69.225 | 208.701 | 1.00 | 35.03 | C |
| ATOM | 2847 | CD2 | LEU | G | 101 | 5.000 | 69.937 | 209.322 | 1.00 | 35.16 | C |
| ATOM | 2851 | C | LEU | G | 101 | 6.146 | 65.714 | 208.460 | 1.00 | 28.81 | C |
| ATOM | 2852 | O | LEU | G | 101 | 5.754 | 64.954 | 207.553 | 1.00 | 30.91 | O |
| ATOM | 2854 | N | TYR | G | 102 | 6.733 | 65.299 | 209.577 | 1.00 | 29.37 | N |
| ATOM | 2855 | CA | TYR | G | 102 | 6.951 | 63.896 | 209.892 | 1.00 | 29.98 | C |
| ATOM | 2857 | CB | TYR | G | 102 | 8.443 | 63.613 | 210.048 | 1.00 | 30.15 | C |
| ATOM | 2860 | CG | TYR | G | 102 | 9.268 | 63.594 | 208.783 | 1.00 | 29.96 | C |
| ATOM | 2861 | CD1 | TYR | G | 102 | 9.575 | 64.765 | 208.103 | 1.00 | 28.76 | C |
| ATOM | 2863 | CE1 | TYR | G | 102 | 10.351 | 64.741 | 206.938 | 1.00 | 30.07 | C |
| ATOM | 2865 | CZ | TYR | G | 102 | 10.844 | 63.537 | 206.475 | 1.00 | 31.46 | C |
| ATOM | 2866 | OH | TYR | G | 102 | 11.632 | 63.485 | 205.346 | 1.00 | 30.76 | O |
| ATOM | 2868 | CE2 | TYR | G | 102 | 10.565 | 62.368 | 207.152 | 1.00 | 30.45 | C |
| ATOM | 2870 | CD2 | TYR | G | 102 | 9.787 | 62.402 | 208.296 | 1.00 | 30.45 | C |
| ATOM | 2872 | C | TYR | G | 102 | 6.308 | 63.581 | 211.232 | 1.00 | 30.82 | C |
| ATOM | 2873 | O | TYR | G | 102 | 6.300 | 64.426 | 212.134 | 1.00 | 30.44 | O |
| ATOM | 2875 | N | ILE | G | 103 | 5.797 | 62.364 | 211.367 | 1.00 | 32.88 | N |
| ATOM | 2876 | CA | ILE | G | 103 | 5.532 | 61.794 | 212.678 | 1.00 | 34.26 | C |
| ATOM | 2878 | CB | ILE | G | 103 | 4.411 | 60.741 | 212.672 | 1.00 | 37.22 | C |
| ATOM | 2880 | CG1 | ILE | G | 103 | 3.112 | 61.338 | 212.156 | 1.00 | 42.68 | C |
| ATOM | 2883 | CD1 | ILE | G | 103 | 1.980 | 60.318 | 212.013 | 1.00 | 43.31 | C |
| ATOM | 2887 | CG2 | ILE | G | 103 | 4.215 | 60.170 | 214.083 | 1.00 | 39.89 | C |
| ATOM | 2891 | C | ILE | G | 103 | 6.784 | 61.092 | 213.162 | 1.00 | 30.82 | C |
| ATOM | 2892 | O | ILE | G | 103 | 7.404 | 60.333 | 212.418 | 1.00 | 31.79 | O |
| ATOM | 2894 | N | ILE | G | 104 | 7.150 | 61.352 | 214.410 | 1.00 | 31.85 | N |
| ATOM | 2895 | CA | ILE | G | 104 | 8.167 | 60.568 | 215.093 | 1.00 | 32.24 | C |
| ATOM | 2897 | CB | ILE | G | 104 | 9.299 | 61.443 | 215.659 | 1.00 | 32.88 | C |
| ATOM | 2899 | CG1 | ILE | G | 104 | 10.040 | 62.161 | 214.521 | 1.00 | 35.22 | C |
| ATOM | 2902 | CD1 | ILE | G | 104 | 9.700 | 63.623 | 214.383 | 1.00 | 38.01 | C |
| ATOM | 2906 | CG2 | ILE | G | 104 | 10.278 | 60.602 | 216.481 | 1.00 | 34.65 | C |
| ATOM | 2910 | C | ILE | G | 104 | 7.443 | 59.800 | 216.194 | 1.00 | 32.66 | C |
| ATOM | 2911 | O | ILE | G | 104 | 6.713 | 60.397 | 216.998 | 1.00 | 32.16 | O |
| ATOM | 2913 | N | TYR | G | 105 | 7.598 | 58.477 | 216.187 | 1.00 | 32.24 | N |
| ATOM | 2914 | CA | TYR | G | 105 | 6.952 | 57.614 | 217.176 | 1.00 | 31.00 | C |
| ATOM | 2916 | CB | TYR | G | 105 | 5.935 | 56.683 | 216.520 | 1.00 | 34.53 | C |
| ATOM | 2919 | CG | TYR | G | 105 | 5.100 | 55.947 | 217.543 | 1.00 | 33.22 | C |
| ATOM | 2920 | CD1 | TYR | G | 105 | 4.099 | 56.609 | 218.235 | 1.00 | 35.90 | C |
| ATOM | 2922 | CE1 | TYR | G | 105 | 3.333 | 55.962 | 219.183 | 1.00 | 37.56 | C |
| ATOM | 2924 | CZ | TYR | G | 105 | 3.569 | 54.633 | 219.466 | 1.00 | 36.48 | C |
| ATOM | 2925 | OH | TYR | G | 105 | 2.775 | 54.008 | 220.413 | 1.00 | 37.56 | O |
| ATOM | 2927 | CE2 | TYR | G | 105 | 4.562 | 53.941 | 218.797 | 1.00 | 33.57 | C |
| ATOM | 2929 | CD2 | TYR | G | 105 | 5.331 | 54.605 | 217.844 | 1.00 | 37.41 | C |
| ATOM | 2931 | C | TYR | G | 105 | 7.996 | 56.785 | 217.909 | 1.00 | 30.39 | C |
| ATOM | 2932 | O | TYR | G | 105 | 8.843 | 56.160 | 217.274 | 1.00 | 29.73 | O |
| ATOM | 2934 | N | LEU | G | 106 | 7.931 | 56.790 | 219.238 | 1.00 | 30.64 | N |
| ATOM | 2935 | CA | LEU | G | 106 | 8.886 | 56.051 | 220.068 | 1.00 | 30.38 | C |
| ATOM | 2937 | CB | LEU | G | 106 | 9.874 | 57.013 | 220.738 | 1.00 | 30.66 | C |
| ATOM | 2940 | CG | LEU | G | 106 | 10.963 | 56.352 | 221.592 | 1.00 | 32.63 | C |
| ATOM | 2942 | CD1 | LEU | G | 106 | 11.849 | 55.457 | 220.731 | 1.00 | 33.08 | C |
| ATOM | 2946 | CD2 | LEU | G | 106 | 11.786 | 57.402 | 222.329 | 1.00 | 34.12 | C |
| ATOM | 2950 | C | LEU | G | 106 | 8.157 | 55.250 | 221.135 | 1.00 | 28.09 | C |
| ATOM | 2951 | O | LEU | G | 106 | 7.270 | 55.771 | 221.814 | 1.00 | 30.21 | O |
| ATOM | 2953 | N | LYS | G | 107 | 8.521 | 53.977 | 221.265 | 1.00 | 31.52 | N |
| ATOM | 2954 | CA | LYS | G | 107 | 8.069 | 53.158 | 222.381 | 1.00 | 32.65 | C |
| ATOM | 2956 | CB | LYS | G | 107 | 6.941 | 52.211 | 221.968 | 1.00 | 35.18 | C |
| ATOM | 2959 | CG | LYS | G | 107 | 7.305 | 51.206 | 220.882 | 1.00 | 38.20 | C |
| ATOM | 2962 | CD | LYS | G | 107 | 6.063 | 50.490 | 220.362 | 1.00 | 39.36 | C |
| ATOM | 2965 | CE | LYS | G | 107 | 5.961 | 49.063 | 220.824 | 1.00 | 42.07 | C |
| ATOM | 2968 | NZ | LYS | G | 107 | 6.641 | 48.156 | 219.853 | 1.00 | 43.82 | N |
| ATOM | 2972 | C | LYS | G | 107 | 9.237 | 52.372 | 222.954 | 1.00 | 31.70 | C |
| ATOM | 2973 | O | LYS | G | 107 | 10.207 | 52.087 | 222.258 | 1.00 | 28.79 | O |
| ATOM | 2975 | N | GLY | G | 108 | 9.133 | 52.029 | 224.228 | 1.00 | 30.03 | N |
| ATOM | 2976 | CA | GLY | G | 108 | 10.175 | 51.247 | 224.889 | 1.00 | 33.43 | C |
| ATOM | 2979 | C | GLY | G | 108 | 9.827 | 50.978 | 226.332 | 1.00 | 32.14 | C |
| ATOM | 2980 | O | GLY | G | 108 | 9.120 | 51.769 | 226.955 | 1.00 | 35.13 | O |
| ATOM | 2982 | N | SER | G | 109 | 10.309 | 49.848 | 226.848 | 1.00 | 34.51 | N |
| ATOM | 2983 | CA | SER | G | 109 | 10.148 | 49.479 | 228.250 | 1.00 | 34.13 | C |
| ATOM | 2985 | CB | SER | G | 109 | 9.357 | 48.178 | 228.374 | 1.00 | 36.14 | C |
| ATOM | 2988 | OG | SER | G | 109 | 8.104 | 48.287 | 227.718 | 1.00 | 38.49 | O |
| ATOM | 2990 | C | SER | G | 109 | 11.524 | 49.311 | 228.888 | 1.00 | 33.56 | C |
| ATOM | 2991 | O | SER | G | 109 | 12.417 | 48.710 | 228.287 | 1.00 | 28.74 | O |
| ATOM | 2993 | N | PHE | G | 110 | 11.678 | 49.842 | 230.101 | 1.00 | 32.10 | N |
| ATOM | 2994 | CA | PHE | G | 110 | 12.960 | 49.864 | 230.807 | 1.00 | 34.97 | C |
| ATOM | 2996 | CB | PHE | G | 110 | 13.438 | 51.308 | 231.002 | 1.00 | 36.89 | C |
| ATOM | 2999 | CG | PHE | G | 110 | 13.644 | 52.067 | 229.717 | 1.00 | 36.45 | C |
| ATOM | 3000 | CD1 | PHE | G | 110 | 12.562 | 52.595 | 229.025 | 1.00 | 37.94 | C |
| ATOM | 3002 | CE1 | PHE | G | 110 | 12.747 | 53.298 | 227.837 | 1.00 | 37.66 | C |
| ATOM | 3004 | CZ | PHE | G | 110 | 14.029 | 53.484 | 227.339 | 1.00 | 37.57 | C |
| ATOM | 3006 | CE2 | PHE | G | 110 | 15.118 | 52.968 | 228.027 | 1.00 | 36.83 | C |
| ATOM | 3008 | CD2 | PHE | G | 110 | 14.921 | 52.264 | 229.208 | 1.00 | 37.66 | C |
| ATOM | 3010 | C | PHE | G | 110 | 12.819 | 49.198 | 232.173 | 1.00 | 36.05 | C |
| ATOM | 3011 | O | PHE | G | 110 | 11.713 | 49.092 | 232.709 | 1.00 | 35.76 | O |
| ATOM | 3013 | N | PHE | G | 111 | 13.944 | 48.758 | 232.731 | 1.00 | 36.58 | N |
| ATOM | 3014 | CA | PHE | G | 111 | 13.968 | 48.157 | 234.062 | 1.00 | 36.34 | C |
| ATOM | 3016 | CB | PHE | G | 111 | 14.953 | 46.981 | 234.103 | 1.00 | 36.17 | C |
| ATOM | 3019 | CG | PHE | G | 111 | 14.643 | 45.884 | 233.114 | 1.00 | 36.28 | C |
| ATOM | 3020 | CD1 | PHE | G | 111 | 13.330 | 45.469 | 232.890 | 1.00 | 36.63 | C |
| ATOM | 3022 | CE1 | PHE | G | 111 | 13.050 | 44.453 | 231.988 | 1.00 | 36.22 | C |
| ATOM | 3024 | CZ | PHE | G | 111 | 14.081 | 43.834 | 231.302 | 1.00 | 36.23 | C |
| ATOM | 3026 | CE2 | PHE | G | 111 | 15.387 | 44.232 | 231.514 | 1.00 | 36.20 | C |
| ATOM | 3028 | CD2 | PHE | G | 111 | 15.666 | 45.251 | 232.419 | 1.00 | 36.23 | C |
| ATOM | 3030 | C | PHE | G | 111 | 14.331 | 49.167 | 235.152 | 1.00 | 37.55 | C |
| ATOM | 3031 | O | PHE | G | 111 | 14.256 | 48.851 | 236.340 | 1.00 | 34.15 | O |
| ATOM | 3033 | N | GLN | G | 112 | 14.727 | 50.373 | 234.746 | 1.00 | 38.25 | N |
| ATOM | 3034 | CA | GLN | G | 112 | 15.070 | 51.434 | 235.688 | 1.00 | 38.92 | C |
| ATOM | 3036 | CB | GLN | G | 112 | 16.580 | 51.453 | 235.965 | 1.00 | 39.42 | C |
| ATOM | 3039 | CG | GLN | G | 112 | 17.468 | 51.552 | 234.724 | 1.00 | 40.30 | C |
| ATOM | 3042 | CD | GLN | G | 112 | 18.945 | 51.348 | 235.041 | 1.00 | 41.41 | C |
| ATOM | 3043 | OE1 | GLN | G | 112 | 19.453 | 51.836 | 236.055 | 1.00 | 41.51 | O |
| ATOM | 3044 | NE2 | GLN | G | 112 | 19.642 | 50.624 | 234.170 | 1.00 | 42.05 | N |
| ATOM | 3047 | C | GLN | G | 112 | 14.606 | 52.790 | 235.172 | 1.00 | 38.86 | C |
| ATOM | 3048 | O | GLN | G | 112 | 14.369 | 52.965 | 233.976 | 1.00 | 37.64 | O |
| ATOM | 3050 | N | GLU | G | 113 | 14.469 | 53.739 | 236.094 | 1.00 | 39.67 | N |
| ATOM | 3051 | CA | GLU | G | 113 | 14.054 | 55.102 | 235.772 | 1.00 | 39.86 | C |
| ATOM | 3053 | CB | GLU | G | 113 | 13.941 | 55.931 | 237.057 | 1.00 | 41.54 | C |
| ATOM | 3056 | CG | GLU | G | 113 | 13.501 | 57.373 | 236.847 | 1.00 | 42.98 | C |
| ATOM | 3059 | CD | GLU | G | 113 | 13.225 | 58.098 | 238.155 | 1.00 | 44.34 | C |
| ATOM | 3060 | OE1 | GLU | G | 113 | 13.950 | 57.858 | 239.147 | 1.00 | 47.27 | O |
| ATOM | 3061 | OE2 | GLU | G | 113 | 12.280 | 58.912 | 238.187 | 1.00 | 47.76 | O |
| ATOM | 3062 | C | GLU | G | 113 | 15.054 | 55.750 | 234.817 | 1.00 | 38.51 | C |
| ATOM | 3063 | O | GLU | G | 113 | 16.262 | 55.737 | 235.068 | 1.00 | 35.69 | O |
| ATOM | 3065 | N | VAL | G | 114 | 14.545 | 56.310 | 233.723 | 1.00 | 39.08 | N |
| ATOM | 3066 | CA | VAL | G | 114 | 15.392 | 56.920 | 232.701 | 1.00 | 39.10 | C |
| ATOM | 3068 | CB | VAL | G | 114 | 15.625 | 55.937 | 231.514 | 1.00 | 39.48 | C |
| ATOM | 3070 | CG1 | VAL | G | 114 | 14.354 | 55.738 | 230.697 | 1.00 | 38.87 | C |
| ATOM | 3074 | CG2 | VAL | G | 114 | 16.760 | 56.417 | 230.624 | 1.00 | 41.42 | C |
| ATOM | 3078 | C | VAL | G | 114 | 14.787 | 58.236 | 232.204 | 1.00 | 38.45 | C |
| ATOM | 3079 | O | VAL | G | 114 | 13.569 | 58.420 | 232.237 | 1.00 | 39.60 | O |
| ATOM | 3081 | N | LYS | G | 115 | 15.657 | 59.148 | 231.772 | 1.00 | 37.36 | N |
| ATOM | 3082 | CA | LYS | G | 115 | 15.258 | 60.408 | 231.148 | 1.00 | 37.42 | C |
| ATOM | 3084 | CB | LYS | G | 115 | 15.832 | 61.608 | 231.915 | 1.00 | 38.31 | C |
| ATOM | 3087 | CG | LYS | G | 115 | 15.189 | 61.853 | 233.278 | 1.00 | 39.01 | C |
| ATOM | 3090 | CD | LYS | G | 115 | 15.756 | 63.093 | 233.963 | 1.00 | 39.29 | C |
| ATOM | 3093 | CE | LYS | G | 115 | 15.161 | 63.281 | 235.355 | 1.00 | 40.26 | C |
| ATOM | 3096 | NZ | LYS | G | 115 | 15.782 | 64.411 | 236.104 | 1.00 | 40.67 | N |
| ATOM | 3100 | C | LYS | G | 115 | 15.773 | 60.407 | 229.713 | 1.00 | 35.77 | C |
| ATOM | 3101 | O | LYS | G | 115 | 16.970 | 60.256 | 229.479 | 1.00 | 36.04 | O |
| ATOM | 3103 | N | ILE | G | 116 | 14.869 | 60.564 | 228.752 | 1.00 | 35.05 | N |
| ATOM | 3104 | CA | ILE | G | 116 | 15.246 | 60.517 | 227.339 | 1.00 | 35.19 | C |
| ATOM | 3106 | CB | ILE | G | 116 | 14.655 | 59.264 | 226.646 | 1.00 | 35.38 | C |
| ATOM | 3108 | CG1 | ILE | G | 116 | 15.273 | 58.008 | 227.281 | 1.00 | 38.41 | C |
| ATOM | 3111 | CD1 | ILE | G | 116 | 14.609 | 56.712 | 226.913 | 1.00 | 39.78 | C |
| ATOM | 3115 | CG2 | ILE | G | 116 | 14.920 | 59.306 | 225.136 | 1.00 | 35.79 | C |
| ATOM | 3119 | C | ILE | G | 116 | 14.863 | 61.810 | 226.615 | 1.00 | 33.36 | C |
| ATOM | 3120 | O | ILE | G | 116 | 13.793 | 62.380 | 226.853 | 1.00 | 32.00 | O |
| ATOM | 3122 | N | ASP | G | 117 | 15.763 | 62.267 | 225.745 | 1.00 | 32.93 | N |
| ATOM | 3123 | CA | ASP | G | 117 | 15.585 | 63.504 | 224.984 | 1.00 | 33.14 | C |
| ATOM | 3125 | CB | ASP | G | 117 | 16.655 | 64.526 | 225.375 | 1.00 | 33.72 | C |
| ATOM | 3128 | CG | ASP | G | 117 | 16.573 | 64.936 | 226.835 | 1.00 | 37.42 | C |
| ATOM | 3129 | OD1 | ASP | G | 117 | 15.516 | 65.447 | 227.255 | 1.00 | 38.72 | O |
| ATOM | 3130 | OD2 | ASP | G | 117 | 17.577 | 64.758 | 227.562 | 1.00 | 40.90 | O |
| ATOM | 3131 | C | ASP | G | 117 | 15.679 | 63.227 | 223.487 | 1.00 | 32.30 | C |
| ATOM | 3132 | O | ASP | G | 117 | 16.507 | 62.426 | 223.057 | 1.00 | 31.68 | O |
| ATOM | 3134 | N | LEU | G | 118 | 14.837 | 63.906 | 222.705 | 1.00 | 31.80 | N |
| ATOM | 3135 | CA | LEU | G | 118 | 14.768 | 63.731 | 221.250 | 1.00 | 32.06 | C |
| ATOM | 3137 | CB | LEU | G | 118 | 13.306 | 63.548 | 220.835 | 1.00 | 30.98 | C |
| ATOM | 3140 | CG | LEU | G | 118 | 12.973 | 63.551 | 219.348 | 1.00 | 31.53 | C |
| ATOM | 3142 | CD1 | LEU | G | 118 | 13.650 | 62.385 | 218.652 | 1.00 | 27.96 | C |
| ATOM | 3146 | CD2 | LEU | G | 118 | 11.469 | 63.488 | 219.169 | 1.00 | 34.35 | C |
| ATOM | 3150 | C | LEU | G | 118 | 15.355 | 64.949 | 220.527 | 1.00 | 31.71 | C |
| ATOM | 3151 | O | LEU | G | 118 | 14.911 | 66.076 | 220.752 | 1.00 | 32.20 | O |
| ATOM | 3153 | N | HIS | G | 119 | 16.343 | 64.707 | 219.666 | 1.00 | 33.18 | N |
| ATOM | 3154 | CA | HIS | G | 119 | 17.034 | 65.757 | 218.913 | 1.00 | 31.88 | C |
| ATOM | 3156 | CB | HIS | G | 119 | 18.553 | 65.622 | 219.064 | 1.00 | 31.06 | C |
| ATOM | 3159 | CG | HIS | G | 119 | 19.036 | 65.693 | 220.477 | 1.00 | 32.32 | C |
| ATOM | 3160 | ND1 | HIS | G | 119 | 18.885 | 64.652 | 221.367 | 1.00 | 32.89 | N |
| ATOM | 3162 | CE1 | HIS | G | 119 | 19.413 | 64.993 | 222.529 | 1.00 | 32.16 | C |
| ATOM | 3164 | NE2 | HIS | G | 119 | 19.907 | 66.212 | 222.422 | 1.00 | 32.84 | N |
| ATOM | 3166 | CD2 | HIS | G | 119 | 19.689 | 66.671 | 221.147 | 1.00 | 30.57 | C |
| ATOM | 3168 | C | HIS | G | 119 | 16.704 | 65.647 | 217.429 | 1.00 | 31.85 | C |
| ATOM | 3169 | O | HIS | G | 119 | 16.567 | 64.544 | 216.903 | 1.00 | 31.96 | O |
| ATOM | 3171 | N | PHE | G | 120 | 16.591 | 66.797 | 216.767 | 1.00 | 32.59 | N |
| ATOM | 3172 | CA | PHE | G | 120 | 16.357 | 66.873 | 215.323 | 1.00 | 34.71 | C |
| ATOM | 3174 | CB | PHE | G | 120 | 15.213 | 67.840 | 215.000 | 1.00 | 37.57 | C |
| ATOM | 3177 | CG | PHE | G | 120 | 13.862 | 67.424 | 215.552 | 1.00 | 36.81 | C |
| ATOM | 3178 | CD1 | PHE | G | 120 | 13.553 | 66.092 | 215.801 | 1.00 | 38.42 | C |
| ATOM | 3180 | CE1 | PHE | G | 120 | 12.307 | 65.734 | 216.296 | 1.00 | 37.91 | C |
| ATOM | 3182 | CZ | PHE | G | 120 | 11.352 | 66.704 | 216.535 | 1.00 | 37.40 | C |
| ATOM | 3184 | CE2 | PHE | G | 120 | 11.638 | 68.027 | 216.278 | 1.00 | 39.44 | C |
| ATOM | 3186 | CD2 | PHE | G | 120 | 12.888 | 68.383 | 215.782 | 1.00 | 38.84 | C |
| ATOM | 3188 | C | PHE | G | 120 | 17.612 | 67.329 | 214.579 | 1.00 | 34.86 | C |
| ATOM | 3189 | O | PHE | G | 120 | 17.694 | 67.207 | 213.357 | 1.00 | 36.36 | O |
| ATOM | 3191 | N | ARG | G | 121 | 18.573 | 67.871 | 215.316 | 1.00 | 36.09 | N |
| ATOM | 3192 | CA | ARG | G | 121 | 19.880 | 68.205 | 214.767 | 1.00 | 37.40 | C |
| ATOM | 3194 | CB | ARG | G | 121 | 19.812 | 69.450 | 213.869 | 1.00 | 37.23 | C |
| ATOM | 3197 | CG | ARG | G | 121 | 20.839 | 69.434 | 212.724 | 1.00 | 37.55 | C |
| ATOM | 3200 | CD | ARG | G | 121 | 20.469 | 70.373 | 211.586 | 1.00 | 38.24 | C |
| ATOM | 3203 | NE | ARG | G | 121 | 20.627 | 71.770 | 211.972 | 1.00 | 38.58 | N |
| ATOM | 3205 | CZ | ARG | G | 121 | 20.694 | 72.789 | 211.119 | 1.00 | 39.83 | C |
| ATOM | 3206 | NH1 | ARG | G | 121 | 20.596 | 72.593 | 209.803 | 1.00 | 38.47 | N |
| ATOM | 3209 | NH2 | ARG | G | 121 | 20.853 | 74.022 | 211.591 | 1.00 | 36.68 | N |
| ATOM | 3212 | C | ARG | G | 121 | 20.848 | 68.415 | 215.922 | 1.00 | 37.87 | C |
| ATOM | 3213 | O | ARG | G | 121 | 20.437 | 68.463 | 217.088 | 1.00 | 33.84 | O |
| ATOM | 3215 | N | GLU | G | 122 | 22.130 | 68.535 | 215.594 | 1.00 | 41.10 | N |
| ATOM | 3216 | CA | GLU | G | 122 | 23.182 | 68.704 | 216.599 | 1.00 | 42.86 | C |
| ATOM | 3218 | CB | GLU | G | 122 | 24.569 | 68.591 | 215.946 | 1.00 | 47.99 | C |
| ATOM | 3221 | CG | GLU | G | 122 | 24.905 | 67.199 | 215.390 | 1.00 | 51.18 | C |
| ATOM | 3224 | CD | GLU | G | 122 | 24.486 | 66.999 | 213.929 | 1.00 | 54.74 | C |
| ATOM | 3225 | OE1 | GLU | G | 122 | 23.344 | 67.365 | 213.560 | 1.00 | 50.63 | O |
| ATOM | 3226 | OE2 | GLU | G | 122 | 25.306 | 66.456 | 213.154 | 1.00 | 58.26 | O |
| ATOM | 3227 | C | GLU | G | 122 | 23.064 | 70.027 | 217.369 | 1.00 | 43.14 | C |
| ATOM | 3228 | O | GLU | G | 122 | 23.489 | 70.113 | 218.522 | 1.00 | 40.39 | O |
| ATOM | 3230 | N | ASP | G | 123 | 22.483 | 71.044 | 216.729 | 1.00 | 43.56 | N |
| ATOM | 3231 | CA | ASP | G | 123 | 22.282 | 72.365 | 217.346 | 1.00 | 44.30 | C |
| ATOM | 3233 | CB | ASP | G | 123 | 22.782 | 73.471 | 216.400 | 1.00 | 44.22 | C |
| ATOM | 3236 | CG | ASP | G | 123 | 22.102 | 73.445 | 215.033 | 1.00 | 43.48 | C |
| ATOM | 3237 | OD1 | ASP | G | 123 | 21.149 | 72.657 | 214.838 | 1.00 | 41.46 | O |
| ATOM | 3238 | OD2 | ASP | G | 123 | 22.531 | 74.218 | 214.148 | 1.00 | 42.03 | O |
| ATOM | 3239 | C | ASP | G | 123 | 20.821 | 72.631 | 217.767 | 1.00 | 45.93 | C |
| ATOM | 3240 | O | ASP | G | 123 | 20.450 | 73.770 | 218.062 | 1.00 | 47.73 | O |
| ATOM | 3242 | N | HIS | G | 124 | 20.014 | 71.570 | 217.802 | 1.00 | 46.41 | N |
| ATOM | 3243 | CA | HIS | G | 124 | 18.601 | 71.634 | 218.186 | 1.00 | 44.93 | C |
| ATOM | 3245 | CB | HIS | G | 124 | 17.850 | 70.482 | 217.502 | 1.00 | 45.45 | C |
| ATOM | 3248 | CG | HIS | G | 124 | 16.412 | 70.342 | 217.901 | 1.00 | 46.19 | C |
| ATOM | 3249 | ND1 | HIS | G | 124 | 15.925 | 69.217 | 218.531 | 1.00 | 45.11 | N |
| ATOM | 3251 | CE1 | HIS | G | 124 | 14.626 | 69.353 | 218.734 | 1.00 | 45.05 | C |
| ATOM | 3253 | NE2 | HIS | G | 124 | 14.251 | 70.527 | 218.259 | 1.00 | 44.42 | N |
| ATOM | 3255 | CD2 | HIS | G | 124 | 15.347 | 71.162 | 217.722 | 1.00 | 46.87 | C |
| ATOM | 3257 | C | HIS | G | 124 | 18.454 | 71.528 | 219.703 | 1.00 | 45.27 | C |
| ATOM | 3258 | O | HIS | G | 124 | 19.131 | 70.718 | 220.344 | 1.00 | 42.61 | O |
| ATOM | 3260 | N | ASN | G | 125 | 17.563 | 72.339 | 220.270 | 1.00 | 44.79 | N |
| ATOM | 3261 | CA | ASN | G | 125 | 17.221 | 72.219 | 221.685 | 1.00 | 45.80 | C |
| ATOM | 3263 | CB | ASN | G | 125 | 16.594 | 73.512 | 222.221 | 1.00 | 49.79 | C |
| ATOM | 3266 | CG | ASN | G | 125 | 17.132 | 73.892 | 223.582 | 1.00 | 53.63 | C |
| ATOM | 3267 | OD1 | ASN | G | 125 | 16.424 | 73.812 | 224.589 | 1.00 | 56.18 | O |
| ATOM | 3268 | ND2 | ASN | G | 125 | 18.402 | 74.294 | 223.626 | 1.00 | 54.31 | N |
| ATOM | 3271 | C | ASN | G | 125 | 16.261 | 71.032 | 221.853 | 1.00 | 42.94 | C |
| ATOM | 3272 | O | ASN | G | 125 | 15.162 | 71.050 | 221.300 | 1.00 | 45.72 | O |
| ATOM | 3274 | N | PRO | G | 126 | 16.664 | 70.006 | 222.627 | 1.00 | 40.72 | N |
| ATOM | 3275 | CA | PRO | G | 126 | 15.965 | 68.721 | 222.554 | 1.00 | 40.30 | C |
| ATOM | 3277 | CB | PRO | G | 126 | 16.906 | 67.755 | 223.296 | 1.00 | 41.72 | C |
| ATOM | 3280 | CG | PRO | G | 126 | 18.069 | 68.555 | 223.755 | 1.00 | 40.81 | C |
| ATOM | 3283 | CD | PRO | G | 126 | 17.731 | 69.988 | 223.641 | 1.00 | 41.40 | C |
| ATOM | 3286 | C | PRO | G | 126 | 14.594 | 68.714 | 223.227 | 1.00 | 39.13 | C |
| ATOM | 3287 | O | PRO | G | 126 | 14.341 | 69.504 | 224.140 | 1.00 | 35.82 | O |
| ATOM | 3288 | N | ILE | G | 127 | 13.725 | 67.815 | 222.770 | 1.00 | 35.49 | N |
| ATOM | 3289 | CA | ILE | G | 127 | 12.432 | 67.593 | 223.403 | 1.00 | 36.73 | C |
| ATOM | 3291 | CB | ILE | G | 127 | 11.385 | 67.089 | 222.384 | 1.00 | 36.04 | C |
| ATOM | 3293 | CG1 | ILE | G | 127 | 11.081 | 68.181 | 221.351 | 1.00 | 38.10 | C |
| ATOM | 3296 | CD1 | ILE | G | 127 | 10.501 | 67.648 | 220.059 | 1.00 | 39.51 | C |
| ATOM | 3300 | CG2 | ILE | G | 127 | 10.101 | 66.663 | 223.089 | 1.00 | 36.84 | C |
| ATOM | 3304 | C | ILE | G | 127 | 12.588 | 66.561 | 224.521 | 1.00 | 34.92 | C |
| ATOM | 3305 | O | ILE | G | 127 | 13.081 | 65.464 | 224.284 | 1.00 | 32.62 | O |
| ATOM | 3307 | N | SER | G | 128 | 12.154 | 66.915 | 225.729 | 1.00 | 34.99 | N |
| ATOM | 3308 | CA | SER | G | 128 | 12.102 | 65.967 | 226.838 | 1.00 | 36.10 | C |
| ATOM | 3310 | CB | SER | G | 128 | 11.972 | 66.703 | 228.174 | 1.00 | 35.99 | C |
| ATOM | 3313 | OG | SER | G | 128 | 11.862 | 65.784 | 229.251 | 1.00 | 35.92 | O |
| ATOM | 3315 | C | SER | G | 128 | 10.910 | 65.038 | 226.642 | 1.00 | 37.07 | C |
| ATOM | 3316 | O | SER | G | 128 | 9.778 | 65.498 | 226.563 | 1.00 | 37.95 | O |
| ATOM | 3318 | N | ILE | G | 129 | 11.165 | 63.736 | 226.561 | 1.00 | 37.63 | N |
| ATOM | 3319 | CA | ILE | G | 129 | 10.102 | 62.758 | 226.323 | 1.00 | 37.49 | C |
| ATOM | 3321 | CB | ILE | G | 129 | 10.658 | 61.488 | 225.637 | 1.00 | 38.10 | C |
| ATOM | 3323 | CG1 | ILE | G | 129 | 11.123 | 61.847 | 224.221 | 1.00 | 38.97 | C |
| ATOM | 3326 | CD1 | ILE | G | 129 | 11.640 | 60.685 | 223.402 | 1.00 | 40.20 | C |
| ATOM | 3330 | CG2 | ILE | G | 129 | 9.597 | 60.371 | 225.604 | 1.00 | 37.97 | C |
| ATOM | 3334 | C | ILE | G | 129 | 9.392 | 62.402 | 227.635 | 1.00 | 37.98 | C |
| ATOM | 3335 | O | ILE | G | 129 | 10.034 | 61.909 | 228.572 | 1.00 | 35.37 | O |
| ATOM | 3337 | N | PRO | G | 130 | 8.070 | 62.665 | 227.719 | 1.00 | 37.06 | N |
| ATOM | 3338 | CA | PRO | G | 130 | 7.338 | 62.271 | 228.933 | 1.00 | 37.60 | C |
| ATOM | 3340 | CB | PRO | G | 130 | 5.905 | 62.761 | 228.669 | 1.00 | 37.90 | C |
| ATOM | 3343 | CG | PRO | G | 130 | 6.012 | 63.740 | 227.553 | 1.00 | 38.22 | C |
| ATOM | 3346 | CD | PRO | G | 130 | 7.192 | 63.336 | 226.744 | 1.00 | 36.72 | C |
| ATOM | 3349 | C | PRO | G | 130 | 7.358 | 60.752 | 229.128 | 1.00 | 37.37 | C |
| ATOM | 3350 | O | PRO | G | 130 | 7.198 | 59.999 | 228.161 | 1.00 | 34.94 | O |
| ATOM | 3351 | N | MET | G | 131 | 7.567 | 60.319 | 230.365 | 1.00 | 35.20 | N |
| ATOM | 3352 | CA | MET | G | 131 | 7.677 | 58.903 | 230.683 | 1.00 | 37.52 | C |
| ATOM | 3354 | CB | MET | G | 131 | 8.933 | 58.654 | 231.515 | 1.00 | 39.48 | C |
| ATOM | 3357 | CG | MET | G | 131 | 10.209 | 59.246 | 230.932 | 1.00 | 41.69 | C |
| ATOM | 3360 | SD | MET | G | 131 | 10.693 | 58.440 | 229.394 | 1.00 | 42.71 | S |
| ATOM | 3361 | CE | MET | G | 131 | 11.223 | 56.849 | 230.036 | 1.00 | 43.15 | C |
| ATOM | 3365 | C | MET | G | 131 | 6.463 | 58.435 | 231.472 | 1.00 | 38.41 | C |
| ATOM | 3366 | O | MET | G | 131 | 5.844 | 59.215 | 232.198 | 1.00 | 38.04 | O |
| ATOM | 3368 | N | LEU | G | 132 | 6.134 | 57.154 | 231.322 | 1.00 | 38.69 | N |
| ATOM | 3369 | CA | LEU | G | 132 | 5.124 | 56.499 | 232.148 | 1.00 | 40.20 | C |
| ATOM | 3371 | CB | LEU | G | 132 | 4.128 | 55.746 | 231.267 | 1.00 | 39.95 | C |
| ATOM | 3374 | CG | LEU | G | 132 | 3.375 | 56.597 | 230.244 | 1.00 | 42.57 | C |
| ATOM | 3376 | CD1 | LEU | G | 132 | 2.560 | 55.710 | 229.312 | 1.00 | 43.03 | C |
| ATOM | 3380 | CD2 | LEU | G | 132 | 2.487 | 57.625 | 230.945 | 1.00 | 43.96 | C |
| ATOM | 3384 | C | LEU | G | 132 | 5.806 | 55.529 | 233.113 | 1.00 | 39.89 | C |
| ATOM | 3385 | O | LEU | G | 132 | 7.007 | 55.270 | 232.995 | 1.00 | 35.31 | O |
| ATOM | 3387 | N | ASN | G | 133 | 5.035 | 55.012 | 234.069 | 1.00 | 41.84 | N |
| ATOM | 3388 | CA | ASN | G | 133 | 5.500 | 53.966 | 234.994 | 1.00 | 43.18 | C |
| ATOM | 3390 | CB | ASN | G | 133 | 5.754 | 52.654 | 234.232 | 1.00 | 45.43 | C |
| ATOM | 3393 | CG | ASN | G | 133 | 4.554 | 52.199 | 233.423 | 1.00 | 47.82 | C |
| ATOM | 3394 | OD1 | ASN | G | 133 | 3.541 | 52.892 | 233.338 | 1.00 | 51.00 | O |
| ATOM | 3395 | ND2 | ASN | G | 133 | 4.667 | 51.022 | 232.819 | 1.00 | 48.46 | N |
| ATOM | 3398 | C | ASN | G | 133 | 6.753 | 54.351 | 235.787 | 1.00 | 42.84 | C |
| ATOM | 3399 | O | ASN | G | 133 | 7.702 | 53.571 | 235.874 | 1.00 | 43.25 | O |
| ATOM | 3401 | N | ASP | G | 134 | 6.743 | 55.550 | 236.366 | 1.00 | 43.66 | N |
| ATOM | 3402 | CA | ASP | G | 134 | 7.888 | 56.080 | 237.125 | 1.00 | 43.77 | C |
| ATOM | 3404 | CB | ASP | G | 134 | 8.054 | 55.337 | 238.461 | 1.00 | 45.66 | C |
| ATOM | 3407 | CG | ASP | G | 134 | 6.892 | 55.570 | 239.415 | 1.00 | 47.30 | C |
| ATOM | 3408 | OD1 | ASP | G | 134 | 6.326 | 56.684 | 239.426 | 1.00 | 48.54 | O |
| ATOM | 3409 | OD2 | ASP | G | 134 | 6.555 | 54.637 | 240.170 | 1.00 | 48.06 | O |
| ATOM | 3410 | C | ASP | G | 134 | 9.198 | 56.043 | 236.321 | 1.00 | 42.84 | C |
| ATOM | 3411 | O | ASP | G | 134 | 10.247 | 55.631 | 236.830 | 1.00 | 40.38 | O |
| ATOM | 3413 | N | GLY | G | 135 | 9.119 | 56.477 | 235.064 | 1.00 | 39.79 | N |
| ATOM | 3414 | CA | GLY | G | 135 | 10.294 | 56.602 | 234.203 | 1.00 | 41.20 | C |
| ATOM | 3417 | C | GLY | G | 135 | 10.740 | 55.332 | 233.493 | 1.00 | 40.43 | C |
| ATOM | 3418 | O | GLY | G | 135 | 11.877 | 55.262 | 233.032 | 1.00 | 41.35 | O |
| ATOM | 3420 | N | ARG | G | 136 | 9.855 | 54.340 | 233.389 | 1.00 | 39.20 | N |
| ATOM | 3421 | CA | ARG | G | 136 | 10.216 | 53.024 | 232.836 | 1.00 | 39.01 | C |
| ATOM | 3423 | CB | ARG | G | 136 | 9.993 | 51.939 | 233.889 | 1.00 | 39.26 | C |
| ATOM | 3426 | CG | ARG | G | 136 | 10.952 | 52.013 | 235.053 | 1.00 | 39.97 | C |
| ATOM | 3429 | CD | ARG | G | 136 | 10.806 | 50.797 | 235.946 | 1.00 | 41.96 | C |
| ATOM | 3432 | NE | ARG | G | 136 | 10.901 | 51.153 | 237.359 | 1.00 | 44.07 | N |
| ATOM | 3434 | CZ | ARG | G | 136 | 10.675 | 50.314 | 238.367 | 1.00 | 44.99 | C |
| ATOM | 3435 | NH1 | ARG | G | 136 | 10.345 | 49.044 | 238.139 | 1.00 | 46.05 | N |
| ATOM | 3438 | NH2 | ARG | G | 136 | 10.781 | 50.749 | 239.615 | 1.00 | 45.05 | N |
| ATOM | 3441 | C | ARG | G | 136 | 9.479 | 52.619 | 231.555 | 1.00 | 37.21 | C |
| ATOM | 3442 | O | ARG | G | 136 | 9.785 | 51.574 | 230.977 | 1.00 | 34.53 | O |
| ATOM | 3444 | N | ARG | G | 137 | 8.505 | 53.411 | 231.121 | 1.00 | 35.93 | N |
| ATOM | 3445 | CA | ARG | G | 137 | 7.820 | 53.132 | 229.862 | 1.00 | 36.46 | C |
| ATOM | 3447 | CB | ARG | G | 137 | 6.458 | 52.479 | 230.104 | 1.00 | 36.52 | C |
| ATOM | 3450 | CG | ARG | G | 137 | 5.773 | 52.060 | 228.815 | 1.00 | 36.06 | C |
| ATOM | 3453 | CD | ARG | G | 137 | 4.401 | 51.482 | 229.045 | 1.00 | 37.15 | C |
| ATOM | 3456 | NE | ARG | G | 137 | 3.816 | 51.074 | 227.772 | 1.00 | 36.40 | N |
| ATOM | 3458 | CZ | ARG | G | 137 | 2.548 | 50.717 | 227.590 | 1.00 | 36.44 | C |
| ATOM | 3459 | NH1 | ARG | G | 137 | 1.689 | 50.688 | 228.603 | 1.00 | 36.26 | N |
| ATOM | 3462 | NH2 | ARG | G | 137 | 2.145 | 50.371 | 226.373 | 1.00 | 36.01 | N |
| ATOM | 3465 | C | ARG | G | 137 | 7.645 | 54.391 | 229.027 | 1.00 | 34.82 | C |
| ATOM | 3466 | O | ARG | G | 137 | 7.272 | 55.445 | 229.545 | 1.00 | 34.17 | O |
| ATOM | 3468 | N | ILE | G | 138 | 7.912 | 54.255 | 227.731 | 1.00 | 34.76 | N |
| ATOM | 3469 | CA | ILE | G | 138 | 7.701 | 55.327 | 226.764 | 1.00 | 35.08 | C |
| ATOM | 3471 | CB | ILE | G | 138 | 8.996 | 55.655 | 225.997 | 1.00 | 35.07 | C |
| ATOM | 3473 | CG1 | ILE | G | 138 | 10.096 | 55.992 | 227.001 | 1.00 | 39.34 | C |
| ATOM | 3476 | CD1 | ILE | G | 138 | 11.350 | 56.555 | 226.395 | 1.00 | 40.25 | C |
| ATOM | 3480 | CG2 | ILE | G | 138 | 8.782 | 56.825 | 225.031 | 1.00 | 35.04 | C |
| ATOM | 3484 | C | ILE | G | 138 | 6.622 | 54.923 | 225.776 | 1.00 | 31.35 | C |
| ATOM | 3485 | O | ILE | G | 138 | 6.633 | 53.814 | 225.244 | 1.00 | 26.44 | O |
| ATOM | 3487 | N | VAL | G | 139 | 5.668 | 55.822 | 225.576 | 1.00 | 31.76 | N |
| ATOM | 3488 | CA | VAL | G | 139 | 4.780 | 55.788 | 224.416 | 1.00 | 34.01 | C |
| ATOM | 3490 | CB | VAL | G | 139 | 3.380 | 55.224 | 224.745 | 1.00 | 35.26 | C |
| ATOM | 3492 | CG1 | VAL | G | 139 | 2.484 | 55.280 | 223.499 | 1.00 | 37.79 | C |
| ATOM | 3496 | CG2 | VAL | G | 139 | 3.481 | 53.806 | 225.272 | 1.00 | 34.86 | C |
| ATOM | 3500 | C | VAL | G | 139 | 4.674 | 57.245 | 223.993 | 1.00 | 34.42 | C |
| ATOM | 3501 | O | VAL | G | 139 | 4.069 | 58.053 | 224.695 | 1.00 | 35.03 | O |
| ATOM | 3503 | N | PHE | G | 140 | 5.302 | 57.595 | 222.877 | 1.00 | 33.64 | N |
| ATOM | 3504 | CA | PHE | G | 140 | 5.494 | 59.004 | 222.549 | 1.00 | 34.03 | C |
| ATOM | 3506 | CB | PHE | G | 140 | 6.897 | 59.450 | 222.966 | 1.00 | 36.15 | C |
| ATOM | 3509 | CG | PHE | G | 140 | 7.190 | 60.887 | 222.658 | 1.00 | 35.11 | C |
| ATOM | 3510 | CD1 | PHE | G | 140 | 6.665 | 61.896 | 223.448 | 1.00 | 38.14 | C |
| ATOM | 3512 | CE1 | PHE | G | 140 | 6.932 | 63.229 | 223.172 | 1.00 | 36.51 | C |
| ATOM | 3514 | CZ | PHE | G | 140 | 7.724 | 63.562 | 222.098 | 1.00 | 37.38 | C |
| ATOM | 3516 | CE2 | PHE | G | 140 | 8.250 | 62.569 | 221.291 | 1.00 | 38.45 | C |
| ATOM | 3518 | CD2 | PHE | G | 140 | 7.980 | 61.234 | 221.571 | 1.00 | 37.69 | C |
| ATOM | 3520 | C | PHE | G | 140 | 5.285 | 59.276 | 221.072 | 1.00 | 33.25 | C |
| ATOM | 3521 | O | PHE | G | 140 | 5.901 | 58.629 | 220.226 | 1.00 | 31.37 | O |
| ATOM | 3523 | N | THR | G | 141 | 4.413 | 60.241 | 220.779 | 1.00 | 34.52 | N |
| ATOM | 3524 | CA | THR | G | 141 | 4.165 | 60.690 | 219.411 | 1.00 | 35.25 | C |
| ATOM | 3526 | CB | THR | G | 141 | 2.701 | 60.449 | 218.987 | 1.00 | 39.10 | C |
| ATOM | 3528 | OG1 | THR | G | 141 | 2.348 | 59.080 | 219.209 | 1.00 | 37.71 | O |
| ATOM | 3530 | CG2 | THR | G | 141 | 2.495 | 60.791 | 217.509 | 1.00 | 39.26 | C |
| ATOM | 3534 | C | THR | G | 141 | 4.450 | 62.186 | 219.308 | 1.00 | 37.64 | C |
| ATOM | 3535 | O | THR | G | 141 | 4.060 | 62.973 | 220.185 | 1.00 | 36.06 | O |
| ATOM | 3537 | N | VAL | G | 142 | 5.158 | 62.562 | 218.248 | 1.00 | 37.01 | N |
| ATOM | 3538 | CA | VAL | G | 142 | 5.305 | 63.961 | 217.870 | 1.00 | 34.85 | C |
| ATOM | 3540 | CB | VAL | G | 142 | 6.660 | 64.574 | 218.350 | 1.00 | 37.60 | C |
| ATOM | 3542 | CG1 | VAL | G | 142 | 7.838 | 63.836 | 217.785 | 1.00 | 37.01 | C |
| ATOM | 3546 | CG2 | VAL | G | 142 | 6.752 | 66.058 | 217.997 | 1.00 | 39.45 | C |
| ATOM | 3550 | C | VAL | G | 142 | 5.151 | 64.096 | 216.358 | 1.00 | 32.27 | C |
| ATOM | 3551 | O | VAL | G | 142 | 5.563 | 63.216 | 215.595 | 1.00 | 31.32 | O |
| ATOM | 3553 | N | VAL | G | 143 | 4.498 | 65.176 | 215.947 | 1.00 | 30.99 | N |
| ATOM | 3554 | CA | VAL | G | 143 | 4.528 | 65.639 | 214.581 | 1.00 | 29.81 | C |
| ATOM | 3556 | CB | VAL | G | 143 | 3.111 | 65.933 | 214.044 | 1.00 | 32.61 | C |
| ATOM | 3558 | CG1 | VAL | G | 143 | 3.166 | 66.272 | 212.558 | 1.00 | 31.79 | C |
| ATOM | 3562 | CG2 | VAL | G | 143 | 2.195 | 64.741 | 214.295 | 1.00 | 31.30 | C |
| ATOM | 3566 | C | VAL | G | 143 | 5.373 | 66.908 | 214.575 | 1.00 | 31.33 | C |
| ATOM | 3567 | O | VAL | G | 143 | 5.189 | 67.783 | 215.430 | 1.00 | 30.24 | O |
| ATOM | 3569 | N | ALA | G | 144 | 6.306 | 66.992 | 213.631 | 1.00 | 31.31 | N |
| ATOM | 3570 | CA | ALA | G | 144 | 7.262 | 68.096 | 213.587 | 1.00 | 33.09 | C |
| ATOM | 3572 | CB | ALA | G | 144 | 8.521 | 67.736 | 214.388 | 1.00 | 33.45 | C |
| ATOM | 3576 | C | ALA | G | 144 | 7.631 | 68.422 | 212.153 | 1.00 | 33.43 | C |
| ATOM | 3577 | O | ALA | G | 144 | 7.631 | 67.545 | 211.286 | 1.00 | 30.07 | O |
| ATOM | 3579 | N | SER | G | 145 | 7.947 | 69.689 | 211.909 | 1.00 | 33.79 | N |
| ATOM | 3580 | CA | SER | G | 145 | 8.485 | 70.106 | 210.628 | 1.00 | 33.60 | C |
| ATOM | 3582 | CB | SER | G | 145 | 8.234 | 71.595 | 210.405 | 1.00 | 35.97 | C |
| ATOM | 3585 | OG | SER | G | 145 | 8.788 | 72.006 | 209.173 | 1.00 | 38.46 | O |
| ATOM | 3587 | C | SER | G | 145 | 9.983 | 69.820 | 210.599 | 1.00 | 33.36 | C |
| ATOM | 3588 | O | SER | G | 145 | 10.740 | 70.369 | 211.396 | 1.00 | 32.20 | O |
| ATOM | 3590 | N | LEU | G | 146 | 10.396 | 68.935 | 209.700 | 1.00 | 33.62 | N |
| ATOM | 3591 | CA | LEU | G | 146 | 11.798 | 68.593 | 209.527 | 1.00 | 32.68 | C |
| ATOM | 3593 | CB | LEU | G | 146 | 11.985 | 67.080 | 209.647 | 1.00 | 32.52 | C |
| ATOM | 3596 | CG | LEU | G | 146 | 11.520 | 66.450 | 210.971 | 1.00 | 31.98 | C |
| ATOM | 3598 | CD1 | LEU | G | 146 | 11.881 | 64.981 | 210.990 | 1.00 | 31.72 | C |
| ATOM | 3602 | CD2 | LEU | G | 146 | 12.147 | 67.156 | 212.167 | 1.00 | 32.69 | C |
| ATOM | 3606 | C | LEU | G | 146 | 12.284 | 69.085 | 208.168 | 1.00 | 32.54 | C |
| ATOM | 3607 | O | LEU | G | 146 | 11.495 | 69.257 | 207.241 | 1.00 | 32.09 | O |
| ATOM | 3609 | N | ALA | G | 147 | 13.586 | 69.319 | 208.059 | 1.00 | 32.14 | N |
| ATOM | 3610 | CA | ALA | G | 147 | 14.184 | 69.774 | 206.810 | 1.00 | 31.97 | C |
| ATOM | 3612 | CB | ALA | G | 147 | 14.456 | 71.275 | 206.878 | 1.00 | 29.54 | C |
| ATOM | 3616 | C | ALA | G | 147 | 15.474 | 69.024 | 206.504 | 1.00 | 32.61 | C |
| ATOM | 3617 | O | ALA | G | 147 | 16.140 | 68.509 | 207.409 | 1.00 | 32.23 | O |
| ATOM | 3619 | N | PHE | G | 148 | 15.825 | 68.977 | 205.220 | 1.00 | 31.06 | N |
| ATOM | 3620 | CA | PHE | G | 148 | 17.142 | 68.515 | 204.785 | 1.00 | 29.92 | C |
| ATOM | 3622 | CB | PHE | G | 148 | 17.399 | 68.980 | 203.339 | 1.00 | 29.49 | C |
| ATOM | 3625 | CG | PHE | G | 148 | 18.801 | 68.741 | 202.854 | 1.00 | 28.01 | C |
| ATOM | 3626 | CD1 | PHE | G | 148 | 19.367 | 67.479 | 202.926 | 1.00 | 28.99 | C |
| ATOM | 3628 | CE1 | PHE | G | 148 | 20.664 | 67.253 | 202.486 | 1.00 | 29.38 | C |
| ATOM | 3630 | CZ | PHE | G | 148 | 21.407 | 68.293 | 201.945 | 1.00 | 30.10 | C |
| ATOM | 3632 | CE2 | PHE | G | 148 | 20.846 | 69.562 | 201.857 | 1.00 | 31.06 | C |
| ATOM | 3634 | CD2 | PHE | G | 148 | 19.548 | 69.778 | 202.309 | 1.00 | 26.97 | C |
| ATOM | 3636 | C | PHE | G | 148 | 18.248 | 69.017 | 205.735 | 1.00 | 29.03 | C |
| ATOM | 3637 | O | PHE | G | 148 | 18.273 | 70.194 | 206.100 | 1.00 | 28.49 | O |
| ATOM | 3639 | N | LYS | G | 149 | 19.137 | 68.097 | 206.121 | 1.00 | 32.45 | N |
| ATOM | 3640 | CA | LYS | G | 149 | 20.263 | 68.316 | 207.057 | 1.00 | 32.23 | C |
| ATOM | 3642 | CB | LYS | G | 149 | 20.903 | 69.703 | 206.926 | 1.00 | 32.52 | C |
| ATOM | 3645 | CG | LYS | G | 149 | 21.538 | 69.953 | 205.571 | 1.00 | 32.22 | C |
| ATOM | 3648 | CD | LYS | G | 149 | 22.185 | 71.313 | 205.516 | 1.00 | 32.59 | C |
| ATOM | 3651 | CE | LYS | G | 149 | 22.654 | 71.641 | 204.113 | 1.00 | 33.38 | C |
| ATOM | 3654 | NZ | LYS | G | 149 | 23.619 | 72.768 | 204.123 | 1.00 | 32.22 | N |
| ATOM | 3658 | C | LYS | G | 149 | 19.921 | 68.009 | 208.523 | 1.00 | 32.79 | C |
| ATOM | 3659 | O | LYS | G | 149 | 20.819 | 67.926 | 209.363 | 1.00 | 29.22 | O |
| ATOM | 3661 | N | ASP | G | 150 | 18.641 | 67.832 | 208.829 | 1.00 | 31.05 | N |
| ATOM | 3662 | CA | ASP | G | 150 | 18.249 | 67.352 | 210.147 | 1.00 | 33.21 | C |
| ATOM | 3664 | CB | ASP | G | 150 | 16.731 | 67.446 | 210.357 | 1.00 | 33.12 | C |
| ATOM | 3667 | CG | ASP | G | 150 | 16.252 | 68.864 | 210.622 | 1.00 | 34.26 | C |
| ATOM | 3668 | OD1 | ASP | G | 150 | 17.089 | 69.761 | 210.864 | 1.00 | 32.00 | O |
| ATOM | 3669 | OD2 | ASP | G | 150 | 15.018 | 69.082 | 210.596 | 1.00 | 33.63 | O |
| ATOM | 3670 | C | ASP | G | 150 | 18.708 | 65.906 | 210.338 | 1.00 | 32.31 | C |
| ATOM | 3671 | O | ASP | G | 150 | 18.809 | 65.126 | 209.381 | 1.00 | 31.03 | O |
| ATOM | 3673 | N | LYS | G | 151 | 18.996 | 65.571 | 211.589 | 1.00 | 32.10 | N |
| ATOM | 3674 | CA | LYS | G | 151 | 19.382 | 64.223 | 211.975 | 1.00 | 34.75 | C |
| ATOM | 3676 | CB | LYS | G | 151 | 20.893 | 64.137 | 212.137 | 1.00 | 37.51 | C |
| ATOM | 3679 | CG | LYS | G | 151 | 21.629 | 64.078 | 210.810 | 1.00 | 39.27 | C |
| ATOM | 3682 | CD | LYS | G | 151 | 23.070 | 64.517 | 210.927 | 1.00 | 41.73 | C |
| ATOM | 3685 | CE | LYS | G | 151 | 23.834 | 64.219 | 209.639 | 1.00 | 43.98 | C |
| ATOM | 3688 | NZ | LYS | G | 151 | 23.008 | 64.469 | 208.419 | 1.00 | 47.72 | N |
| ATOM | 3692 | C | LYS | G | 151 | 18.673 | 63.878 | 213.275 | 1.00 | 32.64 | C |
| ATOM | 3693 | O | LYS | G | 151 | 18.891 | 64.523 | 214.297 | 1.00 | 33.28 | O |
| ATOM | 3695 | N | VAL | G | 152 | 17.814 | 62.868 | 213.228 | 1.00 | 31.61 | N |
| ATOM | 3696 | CA | VAL | G | 152 | 16.975 | 62.528 | 214.373 | 1.00 | 32.20 | C |
| ATOM | 3698 | CB | VAL | G | 152 | 15.583 | 62.046 | 213.928 | 1.00 | 33.20 | C |
| ATOM | 3700 | CG1 | VAL | G | 152 | 14.716 | 61.693 | 215.133 | 1.00 | 33.07 | C |
| ATOM | 3704 | CG2 | VAL | G | 152 | 14.911 | 63.112 | 213.070 | 1.00 | 34.07 | C |
| ATOM | 3708 | C | VAL | G | 152 | 17.651 | 61.477 | 215.250 | 1.00 | 31.20 | C |
| ATOM | 3709 | O | VAL | G | 152 | 17.926 | 60.364 | 214.801 | 1.00 | 28.73 | O |
| ATOM | 3711 | N | TYR | G | 153 | 17.923 | 61.845 | 216.499 | 1.00 | 30.45 | N |
| ATOM | 3712 | CA | TYR | G | 153 | 18.533 | 60.923 | 217.453 | 1.00 | 31.82 | C |
| ATOM | 3714 | CB | TYR | G | 153 | 20.061 | 60.997 | 217.383 | 1.00 | 32.34 | C |
| ATOM | 3717 | CG | TYR | G | 153 | 20.663 | 62.329 | 217.749 | 1.00 | 31.88 | C |
| ATOM | 3718 | CD1 | TYR | G | 153 | 20.720 | 63.369 | 216.823 | 1.00 | 32.51 | C |
| ATOM | 3720 | CE1 | TYR | G | 153 | 21.293 | 64.599 | 217.156 | 1.00 | 31.44 | C |
| ATOM | 3722 | CZ | TYR | G | 153 | 21.821 | 64.785 | 218.420 | 1.00 | 32.42 | C |
| ATOM | 3723 | OH | TYR | G | 153 | 22.389 | 65.987 | 218.761 | 1.00 | 32.82 | O |
| ATOM | 3725 | CE2 | TYR | G | 153 | 21.786 | 63.764 | 219.352 | 1.00 | 33.70 | C |
| ATOM | 3727 | CD2 | TYR | G | 153 | 21.211 | 62.542 | 219.012 | 1.00 | 32.75 | C |
| ATOM | 3729 | C | TYR | G | 153 | 18.037 | 61.132 | 218.880 | 1.00 | 32.10 | C |
| ATOM | 3730 | O | TYR | G | 153 | 17.384 | 62.132 | 219.187 | 1.00 | 32.35 | O |
| ATOM | 3732 | N | LEU | G | 154 | 18.355 | 60.163 | 219.736 | 1.00 | 32.03 | N |
| ATOM | 3733 | CA | LEU | G | 154 | 17.784 | 60.065 | 221.071 | 1.00 | 31.11 | C |
| ATOM | 3735 | CB | LEU | G | 154 | 16.863 | 58.846 | 221.154 | 1.00 | 31.78 | C |
| ATOM | 3738 | CG | LEU | G | 154 | 15.603 | 58.880 | 220.285 | 1.00 | 35.07 | C |
| ATOM | 3740 | CD1 | LEU | G | 154 | 15.169 | 57.469 | 219.907 | 1.00 | 37.39 | C |
| ATOM | 3744 | CD2 | LEU | G | 154 | 14.492 | 59.626 | 220.997 | 1.00 | 34.86 | C |
| ATOM | 3748 | C | LEU | G | 154 | 18.904 | 59.901 | 222.074 | 1.00 | 30.28 | C |
| ATOM | 3749 | O | LEU | G | 154 | 19.659 | 58.935 | 221.991 | 1.00 | 27.43 | O |
| ATOM | 3751 | N | THR | G | 155 | 19.022 | 60.838 | 223.013 | 1.00 | 30.43 | N |
| ATOM | 3752 | CA | THR | G | 155 | 20.010 | 60.716 | 224.079 | 1.00 | 30.24 | C |
| ATOM | 3754 | CB | THR | G | 155 | 20.723 | 62.041 | 224.361 | 1.00 | 30.52 | C |
| ATOM | 3756 | OG1 | THR | G | 155 | 19.757 | 63.025 | 224.738 | 1.00 | 28.71 | O |
| ATOM | 3758 | CG2 | THR | G | 155 | 21.506 | 62.510 | 223.140 | 1.00 | 31.02 | C |
| ATOM | 3762 | C | THR | G | 155 | 19.354 | 60.257 | 225.374 | 1.00 | 31.46 | C |
| ATOM | 3763 | O | THR | G | 155 | 18.166 | 60.494 | 225.603 | 1.00 | 30.61 | O |
| ATOM | 3765 | N | VAL | G | 156 | 20.143 | 59.588 | 226.207 | 1.00 | 32.25 | N |
| ATOM | 3766 | CA | VAL | G | 156 | 19.753 | 59.255 | 227.562 | 1.00 | 38.04 | C |
| ATOM | 3768 | CB | VAL | G | 156 | 20.080 | 57.787 | 227.904 | 1.00 | 39.96 | C |
| ATOM | 3770 | CG1 | VAL | G | 156 | 19.763 | 57.494 | 229.371 | 1.00 | 39.74 | C |
| ATOM | 3774 | CG2 | VAL | G | 156 | 19.324 | 56.840 | 226.974 | 1.00 | 39.26 | C |
| ATOM | 3778 | C | VAL | G | 156 | 20.554 | 60.176 | 228.479 | 1.00 | 40.60 | C |
| ATOM | 3779 | O | VAL | G | 156 | 21.786 | 60.110 | 228.501 | 1.00 | 40.10 | O |
| ATOM | 3781 | N | ASN | G | 157 | 19.861 | 61.045 | 229.211 | 1.00 | 43.59 | N |
| ATOM | 3782 | CA | ASN | G | 157 | 20.518 | 61.919 | 230.182 | 1.00 | 45.58 | C |
| ATOM | 3784 | CB | ASN | G | 157 | 19.619 | 63.115 | 230.526 | 1.00 | 45.61 | C |
| ATOM | 3787 | CG | ASN | G | 157 | 20.333 | 64.179 | 231.354 | 1.00 | 45.86 | C |
| ATOM | 3788 | OD1 | ASN | G | 157 | 21.518 | 64.058 | 231.675 | 1.00 | 46.91 | O |
| ATOM | 3789 | ND2 | ASN | G | 157 | 19.604 | 65.233 | 231.704 | 1.00 | 45.70 | N |
| ATOM | 3792 | C | ASN | G | 157 | 20.880 | 61.113 | 231.435 | 1.00 | 48.32 | C |
| ATOM | 3793 | O | ASN | G | 157 | 20.052 | 60.936 | 232.333 | 1.00 | 50.00 | O |
| ATOM | 3795 | N | ALA | G | 158 | 22.118 | 60.619 | 231.473 | 1.00 | 50.32 | N |
| ATOM | 3796 | CA | ALA | G | 158 | 22.615 | 59.813 | 232.592 | 1.00 | 51.05 | C |
| ATOM | 3798 | CB | ALA | G | 158 | 21.931 | 58.447 | 232.604 | 1.00 | 51.27 | C |
| ATOM | 3802 | C | ALA | G | 158 | 24.135 | 59.638 | 232.502 | 1.00 | 53.29 | C |
| ATOM | 3803 | O | ALA | G | 158 | 24.722 | 59.884 | 231.446 | 1.00 | 54.76 | O |
| ATOM | 3805 | N | PRO | G | 159 | 24.781 | 59.220 | 233.611 | 1.00 | 53.91 | N |
| ATOM | 3806 | CA | PRO | G | 159 | 26.200 | 58.844 | 233.573 | 1.00 | 54.26 | C |
| ATOM | 3808 | CB | PRO | G | 159 | 26.473 | 58.343 | 234.999 | 1.00 | 54.61 | C |
| ATOM | 3811 | CG | PRO | G | 159 | 25.449 | 59.009 | 235.841 | 1.00 | 54.42 | C |
| ATOM | 3814 | CD | PRO | G | 159 | 24.227 | 59.120 | 234.975 | 1.00 | 54.20 | C |
| ATOM | 3817 | C | PRO | G | 159 | 26.516 | 57.744 | 232.551 | 1.00 | 55.54 | C |
| ATOM | 3818 | O | PRO | G | 159 | 25.642 | 56.943 | 232.207 | 1.00 | 55.54 | O |
| ATOM | 3819 | N | ASP | G | 160 | 27.766 | 57.711 | 232.091 | 1.00 | 56.10 | N |
| ATOM | 3820 | CA | ASP | G | 160 | 28.206 | 56.787 | 231.034 | 1.00 | 55.53 | C |
| ATOM | 3822 | CB | ASP | G | 160 | 29.700 | 56.984 | 230.729 | 1.00 | 56.97 | C |
| ATOM | 3825 | CG | ASP | G | 160 | 30.052 | 58.427 | 230.390 | 1.00 | 57.52 | C |
| ATOM | 3826 | OD1 | ASP | G | 160 | 29.480 | 58.974 | 229.425 | 1.00 | 56.60 | O |
| ATOM | 3827 | OD2 | ASP | G | 160 | 30.908 | 59.008 | 231.092 | 1.00 | 58.18 | O |
| ATOM | 3828 | C | ASP | G | 160 | 27.968 | 55.322 | 231.406 | 1.00 | 55.25 | C |
| ATOM | 3829 | O | ASP | G | 160 | 27.533 | 54.527 | 230.573 | 1.00 | 53.83 | O |
| ATOM | 3831 | N | THR | G | 161 | 28.251 | 54.987 | 232.665 | 1.00 | 55.14 | N |
| ATOM | 3832 | CA | THR | G | 161 | 28.188 | 53.611 | 233.169 | 1.00 | 54.26 | C |
| ATOM | 3834 | CB | THR | G | 161 | 28.501 | 53.572 | 234.679 | 1.00 | 55.03 | C |
| ATOM | 3836 | OG1 | THR | G | 161 | 29.751 | 54.226 | 234.926 | 1.00 | 57.09 | O |
| ATOM | 3838 | CG2 | THR | G | 161 | 28.560 | 52.133 | 235.193 | 1.00 | 55.51 | C |
| ATOM | 3842 | C | THR | G | 161 | 26.835 | 52.934 | 232.961 | 1.00 | 52.44 | C |
| ATOM | 3843 | O | THR | G | 161 | 26.767 | 51.788 | 232.515 | 1.00 | 49.87 | O |
| ATOM | 3845 | N | LEU | G | 162 | 25.762 | 53.641 | 233.298 | 1.00 | 51.41 | N |
| ATOM | 3846 | CA | LEU | G | 162 | 24.425 | 53.061 | 233.230 | 1.00 | 51.38 | C |
| ATOM | 3848 | CB | LEU | G | 162 | 23.431 | 53.890 | 234.057 | 1.00 | 53.41 | C |
| ATOM | 3851 | CG | LEU | G | 162 | 23.629 | 53.728 | 235.574 | 1.00 | 55.87 | C |
| ATOM | 3853 | CD1 | LEU | G | 162 | 23.077 | 54.915 | 236.353 | 1.00 | 56.66 | C |
| ATOM | 3857 | CD2 | LEU | G | 162 | 23.014 | 52.424 | 236.080 | 1.00 | 56.38 | C |
| ATOM | 3861 | C | LEU | G | 162 | 23.933 | 52.866 | 231.791 | 1.00 | 49.78 | C |
| ATOM | 3862 | O | LEU | G | 162 | 23.052 | 52.041 | 231.559 | 1.00 | 50.92 | O |
| ATOM | 3864 | N | CYS | G | 163 | 24.511 | 53.597 | 230.832 | 1.00 | 47.92 | N |
| ATOM | 3865 | CA | CYS | G | 163 | 24.146 | 53.436 | 229.416 | 1.00 | 45.83 | C |
| ATOM | 3867 | CB | CYS | G | 163 | 24.712 | 54.569 | 228.546 | 1.00 | 46.95 | C |
| ATOM | 3870 | SG | CYS | G | 163 | 24.632 | 54.253 | 226.742 | 1.00 | 47.29 | S |
| ATOM | 3872 | C | CYS | G | 163 | 24.566 | 52.072 | 228.859 | 1.00 | 42.69 | C |
| ATOM | 3873 | O | CYS | G | 163 | 23.745 | 51.383 | 228.263 | 1.00 | 37.97 | O |
| ATOM | 3875 | N | GLU | G | 164 | 25.820 | 51.660 | 229.066 | 1.00 | 42.98 | N |
| ATOM | 3876 | CA | GLU | G | 164 | 26.260 | 50.347 | 228.548 | 1.00 | 42.18 | C |
| ATOM | 3878 | CB | GLU | G | 164 | 27.789 | 50.153 | 228.592 | 1.00 | 44.40 | C |
| ATOM | 3881 | CG | GLU | G | 164 | 28.499 | 50.581 | 229.869 | 1.00 | 45.32 | C |
| ATOM | 3884 | CD | GLU | G | 164 | 29.337 | 51.842 | 229.693 | 1.00 | 47.32 | C |
| ATOM | 3885 | OE1 | GLU | G | 164 | 28.909 | 52.757 | 228.957 | 1.00 | 48.37 | O |
| ATOM | 3886 | OE2 | GLU | G | 164 | 30.432 | 51.914 | 230.297 | 1.00 | 47.83 | O |
| ATOM | 3887 | C | GLU | G | 164 | 25.545 | 49.157 | 229.203 | 1.00 | 39.22 | C |
| ATOM | 3888 | O | GLU | G | 164 | 25.567 | 48.054 | 228.655 | 1.00 | 40.36 | O |
| ATOM | 3890 | N | HIS | G | 165 | 24.901 | 49.381 | 230.348 | 1.00 | 36.80 | N |
| ATOM | 3891 | CA | HIS | G | 165 | 24.116 | 48.337 | 231.020 | 1.00 | 38.28 | C |
| ATOM | 3893 | CB | HIS | G | 165 | 24.431 | 48.318 | 232.521 | 1.00 | 39.92 | C |
| ATOM | 3896 | CG | HIS | G | 165 | 25.824 | 47.868 | 232.836 | 1.00 | 40.60 | C |
| ATOM | 3897 | ND1 | HIS | G | 165 | 26.273 | 46.595 | 232.558 | 1.00 | 40.60 | N |
| ATOM | 3899 | CE1 | HIS | G | 165 | 27.533 | 46.482 | 232.940 | 1.00 | 42.21 | C |
| ATOM | 3901 | NE2 | HIS | G | 165 | 27.917 | 47.637 | 233.455 | 1.00 | 41.97 | N |
| ATOM | 3903 | CD2 | HIS | G | 165 | 26.867 | 48.520 | 233.404 | 1.00 | 41.10 | C |
| ATOM | 3905 | C | HIS | G | 165 | 22.604 | 48.481 | 230.808 | 1.00 | 36.42 | C |
| ATOM | 3906 | O | HIS | G | 165 | 21.833 | 47.626 | 231.249 | 1.00 | 34.78 | O |
| ATOM | 3908 | N | LEU | G | 166 | 22.181 | 49.548 | 230.133 | 1.00 | 34.82 | N |
| ATOM | 3909 | CA | LEU | G | 166 | 20.759 | 49.793 | 229.898 | 1.00 | 36.67 | C |
| ATOM | 3911 | CB | LEU | G | 166 | 20.535 | 51.199 | 229.327 | 1.00 | 36.79 | C |
| ATOM | 3914 | CG | LEU | G | 166 | 19.118 | 51.763 | 229.470 | 1.00 | 38.00 | C |
| ATOM | 3916 | CD1 | LEU | G | 166 | 18.805 | 52.105 | 230.925 | 1.00 | 39.40 | C |
| ATOM | 3920 | CD2 | LEU | G | 166 | 18.946 | 52.988 | 228.583 | 1.00 | 38.65 | C |
| ATOM | 3924 | C | LEU | G | 166 | 20.186 | 48.747 | 228.937 | 1.00 | 35.83 | C |
| ATOM | 3925 | O | LEU | G | 166 | 20.756 | 48.497 | 227.873 | 1.00 | 35.31 | O |
| ATOM | 3927 | N | GLN | G | 167 | 19.064 | 48.144 | 229.329 | 1.00 | 35.66 | N |
| ATOM | 3928 | CA | GLN | G | 167 | 18.389 | 47.114 | 228.536 | 1.00 | 34.22 | C |
| ATOM | 3930 | CB | GLN | G | 167 | 18.357 | 45.800 | 229.312 | 1.00 | 35.14 | C |
| ATOM | 3933 | CG | GLN | G | 167 | 19.734 | 45.240 | 229.630 | 1.00 | 34.19 | C |
| ATOM | 3936 | CD | GLN | G | 167 | 19.686 | 43.930 | 230.388 | 1.00 | 34.32 | C |
| ATOM | 3937 | OE1 | GLN | G | 167 | 20.611 | 43.122 | 230.300 | 1.00 | 39.47 | O |
| ATOM | 3938 | NE2 | GLN | G | 167 | 18.618 | 43.714 | 231.147 | 1.00 | 30.74 | N |
| ATOM | 3941 | C | GLN | G | 167 | 16.967 | 47.557 | 228.228 | 1.00 | 33.04 | C |
| ATOM | 3942 | O | GLN | G | 167 | 16.232 | 47.960 | 229.131 | 1.00 | 29.93 | O |
| ATOM | 3944 | N | ILE | G | 168 | 16.580 | 47.481 | 226.954 | 1.00 | 33.01 | N |
| ATOM | 3945 | CA | ILE | G | 168 | 15.281 | 47.973 | 226.507 | 1.00 | 33.59 | C |
| ATOM | 3947 | CB | ILE | G | 168 | 15.449 | 49.130 | 225.493 | 1.00 | 34.28 | C |
| ATOM | 3949 | CG1 | ILE | G | 168 | 16.370 | 50.205 | 226.083 | 1.00 | 35.48 | C |
| ATOM | 3952 | CD1 | ILE | G | 168 | 16.866 | 51.213 | 225.100 | 1.00 | 36.50 | C |
| ATOM | 3956 | CG2 | ILE | G | 168 | 14.093 | 49.725 | 225.126 | 1.00 | 34.17 | C |
| ATOM | 3960 | C | ILE | G | 168 | 14.463 | 46.846 | 225.874 | 1.00 | 33.37 | C |
| ATOM | 3961 | O | ILE | G | 168 | 14.953 | 46.136 | 224.990 | 1.00 | 30.67 | O |
| ATOM | 3963 | N | ASN | G | 169 | 13.224 | 46.690 | 226.346 | 1.00 | 31.91 | N |
| ATOM | 3964 | CA | ASN | G | 169 | 12.264 | 45.744 | 225.775 | 1.00 | 32.58 | C |
| ATOM | 3966 | CB | ASN | G | 169 | 11.585 | 44.917 | 226.879 | 1.00 | 31.65 | C |
| ATOM | 3969 | CG | ASN | G | 169 | 12.429 | 43.737 | 227.345 | 1.00 | 31.00 | C |
| ATOM | 3970 | OD1 | ASN | G | 169 | 13.366 | 43.315 | 226.663 | 1.00 | 28.97 | O |
| ATOM | 3971 | ND2 | ASN | G | 169 | 12.086 | 43.187 | 228.508 | 1.00 | 29.46 | N |
| ATOM | 3974 | C | ASN | G | 169 | 11.200 | 46.484 | 224.968 | 1.00 | 32.76 | C |
| ATOM | 3975 | O | ASN | G | 169 | 10.857 | 47.626 | 225.288 | 1.00 | 30.73 | O |
| ATOM | 3977 | N | ASP | G | 170 | 10.689 | 45.830 | 223.925 | 1.00 | 34.94 | N |
| ATOM | 3978 | CA | ASP | G | 170 | 9.603 | 46.377 | 223.102 | 1.00 | 35.82 | C |
| ATOM | 3980 | CB | ASP | G | 170 | 8.287 | 46.337 | 223.896 | 1.00 | 39.67 | C |
| ATOM | 3983 | CG | ASP | G | 170 | 7.046 | 46.466 | 223.018 | 1.00 | 43.95 | C |
| ATOM | 3984 | OD1 | ASP | G | 170 | 7.090 | 46.105 | 221.817 | 1.00 | 47.90 | O |
| ATOM | 3985 | OD2 | ASP | G | 170 | 6.006 | 46.926 | 223.549 | 1.00 | 50.00 | O |
| ATOM | 3986 | C | ASP | G | 170 | 9.932 | 47.803 | 222.637 | 1.00 | 35.76 | C |
| ATOM | 3987 | O | ASP | G | 170 | 9.093 | 48.705 | 222.701 | 1.00 | 37.04 | O |
| ATOM | 3989 | N | GLY | G | 171 | 11.167 | 47.991 | 222.178 | 1.00 | 33.06 | N |
| ATOM | 3990 | CA | GLY | G | 171 | 11.665 | 49.300 | 221.779 | 1.00 | 33.21 | C |
| ATOM | 3993 | C | GLY | G | 171 | 11.612 | 49.488 | 220.277 | 1.00 | 34.16 | C |
| ATOM | 3994 | O | GLY | G | 171 | 12.067 | 48.628 | 219.519 | 1.00 | 32.96 | O |
| ATOM | 3996 | N | GLU | G | 172 | 11.065 | 50.620 | 219.843 | 1.00 | 33.30 | N |
| ATOM | 3997 | CA | GLU | G | 172 | 10.918 | 50.884 | 218.420 | 1.00 | 32.96 | C |
| ATOM | 3999 | CB | GLU | G | 172 | 9.667 | 50.187 | 217.882 | 1.00 | 33.88 | C |
| ATOM | 4002 | CG | GLU | G | 172 | 9.429 | 50.377 | 216.389 | 1.00 | 35.64 | C |
| ATOM | 4005 | CD | GLU | G | 172 | 8.447 | 49.372 | 215.835 | 1.00 | 38.47 | C |
| ATOM | 4006 | OE1 | GLU | G | 172 | 8.860 | 48.215 | 215.606 | 1.00 | 43.84 | O |
| ATOM | 4007 | OE2 | GLU | G | 172 | 7.268 | 49.729 | 215.638 | 1.00 | 38.77 | O |
| ATOM | 4008 | C | GLU | G | 172 | 10.847 | 52.371 | 218.138 | 1.00 | 31.04 | C |
| ATOM | 4009 | O | GLU | G | 172 | 10.158 | 53.107 | 218.846 | 1.00 | 31.70 | O |
| ATOM | 4011 | N | LEU | G | 173 | 11.577 | 52.800 | 217.109 | 1.00 | 30.96 | N |
| ATOM | 4012 | CA | LEU | G | 173 | 11.510 | 54.165 | 216.601 | 1.00 | 30.64 | C |
| ATOM | 4014 | CB | LEU | G | 173 | 12.900 | 54.801 | 216.558 | 1.00 | 31.38 | C |
| ATOM | 4017 | CG | LEU | G | 173 | 12.982 | 56.190 | 215.912 | 1.00 | 30.28 | C |
| ATOM | 4019 | CD1 | LEU | G | 173 | 12.193 | 57.218 | 216.714 | 1.00 | 31.20 | C |
| ATOM | 4023 | CD2 | LEU | G | 173 | 14.419 | 56.620 | 215.755 | 1.00 | 33.38 | C |
| ATOM | 4027 | C | LEU | G | 173 | 10.946 | 54.136 | 215.190 | 1.00 | 30.34 | C |
| ATOM | 4028 | O | LEU | G | 173 | 11.423 | 53.368 | 214.352 | 1.00 | 29.59 | O |
| ATOM | 4030 | N | ILE | G | 174 | 9.930 | 54.960 | 214.936 | 1.00 | 30.68 | N |
| ATOM | 4031 | CA | ILE | G | 174 | 9.421 | 55.164 | 213.583 | 1.00 | 30.14 | C |
| ATOM | 4033 | CB | ILE | G | 174 | 7.974 | 54.624 | 213.388 | 1.00 | 31.16 | C |
| ATOM | 4035 | CG1 | ILE | G | 174 | 7.971 | 53.094 | 213.463 | 1.00 | 33.41 | C |
| ATOM | 4038 | CD1 | ILE | G | 174 | 6.603 | 52.441 | 213.438 | 1.00 | 34.23 | C |
| ATOM | 4042 | CG2 | ILE | G | 174 | 7.411 | 55.088 | 212.039 | 1.00 | 33.28 | C |
| ATOM | 4046 | C | ILE | G | 174 | 9.477 | 56.648 | 213.243 | 1.00 | 30.57 | C |
| ATOM | 4047 | O | ILE | G | 174 | 9.062 | 57.492 | 214.033 | 1.00 | 29.00 | O |
| ATOM | 4049 | N | VAL | G | 175 | 10.009 | 56.944 | 212.063 | 1.00 | 32.41 | N |
| ATOM | 4050 | CA | VAL | G | 175 | 9.982 | 58.277 | 211.482 | 1.00 | 30.75 | C |
| ATOM | 4052 | CB | VAL | G | 175 | 11.408 | 58.864 | 211.352 | 1.00 | 31.43 | C |
| ATOM | 4054 | CG1 | VAL | G | 175 | 11.362 | 60.297 | 210.808 | 1.00 | 33.05 | C |
| ATOM | 4058 | CG2 | VAL | G | 175 | 12.125 | 58.844 | 212.706 | 1.00 | 31.61 | C |
| ATOM | 4062 | C | VAL | G | 175 | 9.321 | 58.146 | 210.108 | 1.00 | 30.19 | C |
| ATOM | 4063 | O | VAL | G | 175 | 9.753 | 57.347 | 209.279 | 1.00 | 32.01 | O |
| ATOM | 4065 | N | VAL | G | 176 | 8.258 | 58.906 | 209.879 | 1.00 | 29.88 | N |
| ATOM | 4066 | CA | VAL | G | 176 | 7.494 | 58.804 | 208.636 | 1.00 | 30.44 | C |
| ATOM | 4068 | CB | VAL | G | 176 | 6.310 | 57.793 | 208.767 | 1.00 | 34.76 | C |
| ATOM | 4070 | CG1 | VAL | G | 176 | 5.398 | 58.144 | 209.933 | 1.00 | 38.16 | C |
| ATOM | 4074 | CG2 | VAL | G | 176 | 5.521 | 57.701 | 207.453 | 1.00 | 32.47 | C |
| ATOM | 4078 | C | VAL | G | 176 | 7.003 | 60.179 | 208.172 | 1.00 | 30.80 | C |
| ATOM | 4079 | O | VAL | G | 176 | 6.432 | 60.942 | 208.952 | 1.00 | 31.78 | O |
| ATOM | 4081 | N | GLN | G | 177 | 7.249 | 60.473 | 206.896 | 1.00 | 29.01 | N |
| ATOM | 4082 | CA | GLN | G | 177 | 6.882 | 61.730 | 206.264 | 1.00 | 28.98 | C |
| ATOM | 4084 | CB | GLN | G | 177 | 7.684 | 61.879 | 204.977 | 1.00 | 29.97 | C |
| ATOM | 4087 | CG | GLN | G | 177 | 7.558 | 63.205 | 204.250 | 1.00 | 30.61 | C |
| ATOM | 4090 | CD | GLN | G | 177 | 8.265 | 63.164 | 202.914 | 1.00 | 31.18 | C |
| ATOM | 4091 | OE1 | GLN | G | 177 | 8.364 | 62.105 | 202.301 | 1.00 | 29.35 | O |
| ATOM | 4092 | NE2 | GLN | G | 177 | 8.760 | 64.312 | 202.454 | 1.00 | 31.97 | N |
| ATOM | 4095 | C | GLN | G | 177 | 5.385 | 61.766 | 205.950 | 1.00 | 32.27 | C |
| ATOM | 4096 | O | GLN | G | 177 | 4.837 | 60.794 | 205.434 | 1.00 | 29.85 | O |
| ATOM | 4098 | N | LEU | G | 178 | 4.749 | 62.891 | 206.280 | 1.00 | 30.33 | N |
| ATOM | 4099 | CA | LEU | G | 178 | 3.310 | 63.121 | 206.087 | 1.00 | 29.78 | C |
| ATOM | 4101 | CB | LEU | G | 178 | 2.713 | 63.750 | 207.357 | 1.00 | 32.52 | C |
| ATOM | 4104 | CG | LEU | G | 178 | 2.081 | 62.851 | 208.423 | 1.00 | 35.40 | C |
| ATOM | 4106 | CD1 | LEU | G | 178 | 2.670 | 61.462 | 208.512 | 1.00 | 36.75 | C |
| ATOM | 4110 | CD2 | LEU | G | 178 | 2.072 | 63.553 | 209.770 | 1.00 | 34.10 | C |
| ATOM | 4114 | C | LEU | G | 178 | 2.988 | 64.031 | 204.899 | 1.00 | 30.40 | C |
| ATOM | 4115 | O | LEU | G | 178 | 1.918 | 63.917 | 204.279 | 1.00 | 26.95 | O |
| ATOM | 4117 | N | THR | G | 179 | 3.884 | 64.965 | 204.613 | 1.00 | 31.34 | N |
| ATOM | 4118 | CA | THR | G | 179 | 3.689 | 65.907 | 203.518 | 1.00 | 30.29 | C |
| ATOM | 4120 | CB | THR | G | 179 | 3.357 | 67.309 | 204.040 | 1.00 | 31.21 | C |
| ATOM | 4122 | OG1 | THR | G | 179 | 4.396 | 67.743 | 204.922 | 1.00 | 31.44 | O |
| ATOM | 4124 | CG2 | THR | G | 179 | 2.026 | 67.310 | 204.782 | 1.00 | 33.31 | C |
| ATOM | 4128 | C | THR | G | 179 | 4.970 | 65.966 | 202.693 | 1.00 | 32.79 | C |
| ATOM | 4129 | O | THR | G | 179 | 6.066 | 65.818 | 203.249 | 1.00 | 33.43 | O |
| ATOM | 4131 | N | PRO | G | 180 | 4.840 | 66.178 | 201.372 | 1.00 | 32.87 | N |
| ATOM | 4132 | CA | PRO | G | 180 | 6.007 | 66.184 | 200.498 | 1.00 | 33.28 | C |
| ATOM | 4134 | CB | PRO | G | 180 | 5.396 | 66.019 | 199.109 | 1.00 | 36.99 | C |
| ATOM | 4137 | CG | PRO | G | 180 | 4.081 | 66.712 | 199.210 | 1.00 | 36.57 | C |
| ATOM | 4140 | CD | PRO | G | 180 | 3.596 | 66.430 | 200.618 | 1.00 | 34.18 | C |
| ATOM | 4143 | C | PRO | G | 180 | 6.763 | 67.497 | 200.592 | 1.00 | 33.62 | C |
| ATOM | 4144 | O | PRO | G | 180 | 6.225 | 68.494 | 201.077 | 1.00 | 32.32 | O |
| ATOM | 4145 | N | GLY | G | 181 | 8.006 | 67.493 | 200.132 | 1.00 | 32.40 | N |
| ATOM | 4146 | CA | GLY | G | 181 | 8.810 | 68.697 | 200.133 | 1.00 | 31.93 | C |
| ATOM | 4149 | C | GLY | G | 181 | 10.079 | 68.511 | 199.346 | 1.00 | 31.94 | C |
| ATOM | 4150 | O | GLY | G | 181 | 10.295 | 67.463 | 198.724 | 1.00 | 30.76 | O |
| ATOM | 4152 | N | TYR | G | 182 | 10.911 | 69.543 | 199.381 | 1.00 | 30.99 | N |
| ATOM | 4153 | CA | TYR | G | 182 | 12.234 | 69.518 | 198.769 | 1.00 | 30.37 | C |
| ATOM | 4155 | CB | TYR | G | 182 | 12.992 | 70.796 | 199.155 | 1.00 | 29.05 | C |
| ATOM | 4158 | CG | TYR | G | 182 | 14.480 | 70.724 | 198.928 | 1.00 | 28.96 | C |
| ATOM | 4159 | CD1 | TYR | G | 182 | 15.034 | 71.046 | 197.688 | 1.00 | 30.22 | C |
| ATOM | 4161 | CE1 | TYR | G | 182 | 16.398 | 70.963 | 197.476 | 1.00 | 28.29 | C |
| ATOM | 4163 | CZ | TYR | G | 182 | 17.230 | 70.559 | 198.510 | 1.00 | 28.04 | C |
| ATOM | 4164 | OH | TYR | G | 182 | 18.592 | 70.473 | 198.316 | 1.00 | 28.02 | O |
| ATOM | 4166 | CE2 | TYR | G | 182 | 16.707 | 70.230 | 199.738 | 1.00 | 28.48 | C |
| ATOM | 4168 | CD2 | TYR | G | 182 | 15.338 | 70.319 | 199.946 | 1.00 | 29.33 | C |
| ATOM | 4170 | C | TYR | G | 182 | 13.014 | 68.289 | 199.223 | 1.00 | 30.19 | C |
| ATOM | 4171 | O | TYR | G | 182 | 13.049 | 67.991 | 200.414 | 1.00 | 28.55 | O |
| ATOM | 4173 | N | CYS | G | 183 | 13.614 | 67.574 | 198.272 | 1.00 | 31.35 | N |
| ATOM | 4174 | CA | CYS | G | 183 | 14.551 | 66.489 | 198.573 | 1.00 | 32.86 | C |
| ATOM | 4176 | CB | CYS | G | 183 | 14.069 | 65.146 | 198.004 | 1.00 | 37.54 | C |
| ATOM | 4179 | SG | CYS | G | 183 | 12.511 | 64.498 | 198.725 | 1.00 | 43.21 | S |
| ATOM | 4181 | C | CYS | G | 183 | 15.902 | 66.830 | 197.973 | 1.00 | 31.59 | C |
| ATOM | 4182 | O | CYS | G | 183 | 15.988 | 67.251 | 196.818 | 1.00 | 29.27 | O |
| ATOM | 4184 | N | ALA | G | 184 | 16.956 | 66.658 | 198.762 | 1.00 | 31.83 | N |
| ATOM | 4185 | CA | ALA | G | 184 | 18.314 | 66.821 | 198.266 | 1.00 | 32.88 | C |
| ATOM | 4187 | CB | ALA | G | 184 | 19.283 | 66.960 | 199.424 | 1.00 | 31.71 | C |
| ATOM | 4191 | C | ALA | G | 184 | 18.655 | 65.590 | 197.432 | 1.00 | 34.21 | C |
| ATOM | 4192 | O | ALA | G | 184 | 18.550 | 64.473 | 197.934 | 1.00 | 35.70 | O |
| ATOM | 4194 | N | PRO | G | 185 | 19.028 | 65.781 | 196.151 | 1.00 | 40.34 | N |
| ATOM | 4195 | CA | PRO | G | 185 | 19.434 | 64.650 | 195.301 | 1.00 | 41.97 | C |
| ATOM | 4197 | CB | PRO | G | 185 | 19.850 | 65.325 | 193.990 | 1.00 | 43.06 | C |
| ATOM | 4200 | CG | PRO | G | 185 | 19.120 | 66.614 | 193.977 | 1.00 | 43.53 | C |
| ATOM | 4203 | CD | PRO | G | 185 | 19.060 | 67.055 | 195.405 | 1.00 | 41.57 | C |
| ATOM | 4206 | C | PRO | G | 185 | 20.605 | 63.847 | 195.870 | 1.00 | 44.95 | C |
| ATOM | 4207 | O | PRO | G | 185 | 21.515 | 64.424 | 196.467 | 1.00 | 45.80 | O |
| ATOM | 4208 | N | GLU | G | 186 | 20.576 | 62.531 | 195.677 | 1.00 | 46.09 | N |
| ATOM | 4209 | CA | GLU | G | 186 | 21.618 | 61.645 | 196.195 | 1.00 | 45.81 | C |
| ATOM | 4211 | CB | GLU | G | 186 | 21.292 | 60.205 | 195.965 | 0.00 | 30.00 | C |
| ATOM | 4214 | CG | GLU | G | 186 | 20.067 | 59.805 | 196.785 | 0.00 | 30.00 | C |
| ATOM | 4217 | CD | GLU | G | 186 | 19.734 | 58.331 | 196.563 | 0.00 | 30.00 | C |
| ATOM | 4218 | OE1 | GLU | G | 186 | 20.305 | 57.716 | 195.662 | 0.00 | 30.00 | O |
| ATOM | 4219 | OE2 | GLU | G | 186 | 18.900 | 57.787 | 197.290 | 0.00 | 30.00 | O |
| ATOM | 4220 | C | GLU | G | 186 | 22.989 | 61.982 | 195.612 | 1.00 | 46.06 | C |
| ATOM | 4221 | O | GLU | G | 186 | 23.131 | 62.118 | 194.396 | 1.00 | 46.72 | O |
| ATOM | 4223 | N | GLY | G | 187 | 23.982 | 62.136 | 196.488 | 1.00 | 46.95 | N |
| ATOM | 4224 | CA | GLY | G | 187 | 25.363 | 62.418 | 196.082 | 1.00 | 47.72 | C |
| ATOM | 4227 | C | GLY | G | 187 | 25.687 | 63.868 | 195.741 | 1.00 | 48.91 | C |
| ATOM | 4228 | O | GLY | G | 187 | 26.772 | 64.152 | 195.228 | 1.00 | 47.34 | O |
| ATOM | 4230 | N | SER | G | 188 | 24.771 | 64.791 | 196.042 | 1.00 | 49.06 | N |
| ATOM | 4231 | CA | SER | G | 188 | 24.924 | 66.200 | 195.645 | 1.00 | 49.26 | C |
| ATOM | 4233 | CB | SER | G | 188 | 23.620 | 66.713 | 195.027 | 1.00 | 49.38 | C |
| ATOM | 4236 | OG | SER | G | 188 | 22.565 | 66.711 | 195.977 | 1.00 | 50.89 | O |
| ATOM | 4238 | C | SER | G | 188 | 25.358 | 67.146 | 196.777 | 1.00 | 49.22 | C |
| ATOM | 4239 | O | SER | G | 188 | 25.658 | 68.317 | 196.518 | 1.00 | 46.47 | O |
| ATOM | 4241 | N | TYR | G | 189 | 25.375 | 66.656 | 198.019 | 1.00 | 50.65 | N |
| ATOM | 4242 | CA | TYR | G | 189 | 25.810 | 67.464 | 199.165 | 1.00 | 51.82 | C |
| ATOM | 4244 | CB | TYR | G | 189 | 24.608 | 68.010 | 199.950 | 1.00 | 50.78 | C |
| ATOM | 4247 | CG | TYR | G | 189 | 25.024 | 68.911 | 201.098 | 1.00 | 50.42 | C |
| ATOM | 4248 | CD1 | TYR | G | 189 | 25.245 | 70.271 | 200.896 | 1.00 | 49.87 | C |
| ATOM | 4250 | CE1 | TYR | G | 189 | 25.651 | 71.096 | 201.937 | 1.00 | 50.54 | C |
| ATOM | 4252 | CZ | TYR | G | 189 | 25.846 | 70.561 | 203.201 | 1.00 | 51.14 | C |
| ATOM | 4253 | OH | TYR | G | 189 | 26.243 | 71.375 | 204.239 | 1.00 | 50.35 | O |
| ATOM | 4255 | CE2 | TYR | G | 189 | 25.635 | 69.211 | 203.427 | 1.00 | 51.13 | C |
| ATOM | 4257 | CD2 | TYR | G | 189 | 25.229 | 68.394 | 202.377 | 1.00 | 50.71 | C |
| ATOM | 4259 | C | TYR | G | 189 | 26.723 | 66.680 | 200.113 | 1.00 | 53.17 | C |
| ATOM | 4260 | O | TYR | G | 189 | 26.475 | 65.507 | 200.397 | 1.00 | 52.31 | O |
| ATOM | 4262 | N | HIS | G | 190 | 27.768 | 67.349 | 200.603 | 1.00 | 54.42 | N |
| ATOM | 4263 | CA | HIS | G | 190 | 28.678 | 66.782 | 201.603 | 1.00 | 56.64 | C |
| ATOM | 4265 | CB | HIS | G | 190 | 29.963 | 66.278 | 200.939 | 1.00 | 60.60 | C |
| ATOM | 4268 | CG | HIS | G | 190 | 29.754 | 65.097 | 200.041 | 1.00 | 62.76 | C |
| ATOM | 4269 | ND1 | HIS | G | 190 | 29.933 | 65.158 | 198.675 | 1.00 | 63.61 | N |
| ATOM | 4271 | CE1 | HIS | G | 190 | 29.676 | 63.974 | 198.147 | 1.00 | 64.44 | C |
| ATOM | 4273 | NE2 | HIS | G | 190 | 29.331 | 63.149 | 199.120 | 1.00 | 64.84 | N |
| ATOM | 4275 | CD2 | HIS | G | 190 | 29.372 | 63.827 | 200.315 | 1.00 | 63.82 | C |
| ATOM | 4277 | C | HIS | G | 190 | 29.018 | 67.813 | 202.683 | 1.00 | 57.39 | C |
| ATOM | 4278 | O | HIS | G | 190 | 29.100 | 69.014 | 202.414 | 1.00 | 57.33 | O |
| ATOM | 4280 | N | LEU | R | 29 | 28.588 | 105.309 | 149.054 | 1.00 | 46.27 | N |
| ATOM | 4281 | CA | LEU | R | 29 | 27.939 | 104.381 | 150.031 | 1.00 | 43.68 | C |
| ATOM | 4283 | CB | LEU | R | 29 | 28.681 | 103.039 | 150.074 | 1.00 | 44.65 | C |
| ATOM | 4286 | CG | LEU | R | 29 | 28.126 | 101.970 | 151.023 | 1.00 | 44.57 | C |
| ATOM | 4288 | CD1 | LEU | R | 29 | 26.685 | 101.634 | 150.677 | 1.00 | 45.42 | C |
| ATOM | 4292 | CD2 | LEU | R | 29 | 28.994 | 100.720 | 150.991 | 1.00 | 43.98 | C |
| ATOM | 4296 | C | LEU | R | 29 | 27.906 | 104.996 | 151.426 | 1.00 | 42.91 | C |
| ATOM | 4297 | O | LEU | R | 29 | 28.923 | 105.499 | 151.908 | 1.00 | 45.02 | O |
| ATOM | 4301 | N | HIS | R | 30 | 26.740 | 104.957 | 152.069 | 1.00 | 42.60 | N |
| ATOM | 4302 | CA | HIS | R | 30 | 26.615 | 105.416 | 153.448 | 1.00 | 41.72 | C |
| ATOM | 4304 | CB | HIS | R | 30 | 26.309 | 106.908 | 153.517 | 1.00 | 46.72 | C |
| ATOM | 4307 | CG | HIS | R | 30 | 26.477 | 107.479 | 154.890 | 1.00 | 48.60 | C |
| ATOM | 4308 | ND1 | HIS | R | 30 | 25.476 | 107.445 | 155.837 | 1.00 | 51.14 | N |
| ATOM | 4310 | CE1 | HIS | R | 30 | 25.911 | 108.002 | 156.953 | 1.00 | 51.61 | C |
| ATOM | 4312 | NE2 | HIS | R | 30 | 27.160 | 108.389 | 156.766 | 1.00 | 51.53 | N |
| ATOM | 4314 | CD2 | HIS | R | 30 | 27.541 | 108.066 | 155.487 | 1.00 | 50.82 | C |
| ATOM | 4316 | C | HIS | R | 30 | 25.559 | 104.640 | 154.227 | 1.00 | 39.91 | C |
| ATOM | 4317 | O | HIS | R | 30 | 24.370 | 104.684 | 153.901 | 1.00 | 38.06 | O |
| ATOM | 4319 | N | CYS | R | 31 | 26.015 | 103.957 | 155.277 | 1.00 | 35.54 | N |
| ATOM | 4320 | CA | CYS | R | 31 | 25.184 | 103.060 | 156.069 | 1.00 | 35.67 | C |
| ATOM | 4322 | CB | CYS | R | 31 | 25.886 | 101.707 | 156.215 | 1.00 | 36.19 | C |
| ATOM | 4325 | SG | CYS | R | 31 | 26.282 | 100.928 | 154.640 | 1.00 | 38.52 | S |
| ATOM | 4327 | C | CYS | R | 31 | 24.912 | 103.647 | 157.447 | 1.00 | 34.00 | C |
| ATOM | 4328 | O | CYS | R | 31 | 25.673 | 104.486 | 157.936 | 1.00 | 34.26 | O |
| ATOM | 4330 | N | VAL | R | 32 | 23.824 | 103.193 | 158.065 | 1.00 | 33.47 | N |
| ATOM | 4331 | CA | VAL | R | 32 | 23.439 | 103.622 | 159.410 | 1.00 | 35.34 | C |
| ATOM | 4333 | CB | VAL | R | 32 | 22.363 | 104.746 | 159.371 | 1.00 | 35.78 | C |
| ATOM | 4335 | CG1 | VAL | R | 32 | 22.912 | 105.984 | 158.679 | 1.00 | 34.85 | C |
| ATOM | 4339 | CG2 | VAL | R | 32 | 21.078 | 104.265 | 158.682 | 1.00 | 35.40 | C |
| ATOM | 4343 | C | VAL | R | 32 | 22.906 | 102.440 | 160.223 | 1.00 | 37.00 | C |
| ATOM | 4344 | O | VAL | R | 32 | 22.592 | 101.388 | 159.669 | 1.00 | 37.17 | O |
| ATOM | 4346 | N | GLY | R | 33 | 22.811 | 102.622 | 161.537 | 1.00 | 36.26 | N |
| ATOM | 4347 | CA | GLY | R | 33 | 22.196 | 101.627 | 162.417 | 1.00 | 36.56 | C |
| ATOM | 4350 | C | GLY | R | 33 | 22.966 | 100.324 | 162.551 | 1.00 | 37.35 | C |
| ATOM | 4351 | O | GLY | R | 33 | 24.193 | 100.327 | 162.676 | 1.00 | 39.86 | O |
| ATOM | 4353 | N | ASP | R | 34 | 22.240 | 99.208 | 162.505 | 1.00 | 34.47 | N |
| ATOM | 4354 | CA | ASP | R | 34 | 22.807 | 97.878 | 162.735 | 1.00 | 33.97 | C |
| ATOM | 4356 | CB | ASP | R | 34 | 21.732 | 96.953 | 163.311 | 1.00 | 37.54 | C |
| ATOM | 4359 | CG | ASP | R | 34 | 21.188 | 97.439 | 164.651 | 1.00 | 40.50 | C |
| ATOM | 4360 | OD1 | ASP | R | 34 | 21.905 | 98.170 | 165.366 | 1.00 | 43.53 | O |
| ATOM | 4361 | OD2 | ASP | R | 34 | 20.041 | 97.081 | 164.991 | 1.00 | 41.97 | O |
| ATOM | 4362 | C | ASP | R | 34 | 23.374 | 97.278 | 161.453 | 1.00 | 31.16 | C |
| ATOM | 4363 | O | ASP | R | 34 | 23.125 | 96.115 | 161.133 | 1.00 | 31.70 | O |
| ATOM | 4365 | N | THR | R | 35 | 24.132 | 98.085 | 160.717 | 1.00 | 29.60 | N |
| ATOM | 4366 | CA | THR | R | 35 | 24.695 | 97.667 | 159.447 | 1.00 | 30.50 | C |
| ATOM | 4368 | CB | THR | R | 35 | 23.928 | 98.297 | 158.254 | 1.00 | 32.36 | C |
| ATOM | 4370 | OG1 | THR | R | 35 | 24.007 | 99.722 | 158.325 | 1.00 | 32.83 | O |
| ATOM | 4372 | CG2 | THR | R | 35 | 22.456 | 97.869 | 158.265 | 1.00 | 35.06 | C |
| ATOM | 4376 | C | THR | R | 35 | 26.173 | 98.031 | 159.345 | 1.00 | 28.90 | C |
| ATOM | 4377 | O | THR | R | 35 | 26.710 | 98.762 | 160.179 | 1.00 | 27.73 | O |
| ATOM | 4379 | N | TYR | R | 36 | 26.828 | 97.484 | 158.332 | 1.00 | 27.80 | N |
| ATOM | 4380 | CA | TYR | R | 36 | 28.199 | 97.850 | 158.024 | 1.00 | 27.30 | C |
| ATOM | 4382 | CB | TYR | R | 36 | 29.187 | 96.882 | 158.693 | 1.00 | 27.22 | C |
| ATOM | 4385 | CG | TYR | R | 36 | 29.044 | 95.435 | 158.274 | 1.00 | 23.58 | C |
| ATOM | 4386 | CD1 | TYR | R | 36 | 28.102 | 94.608 | 158.869 | 1.00 | 24.91 | C |
| ATOM | 4388 | CE1 | TYR | R | 36 | 27.966 | 93.272 | 158.490 | 1.00 | 27.21 | C |
| ATOM | 4390 | CZ | TYR | R | 36 | 28.787 | 92.751 | 157.504 | 1.00 | 28.43 | C |
| ATOM | 4391 | OH | TYR | R | 36 | 28.657 | 91.422 | 157.133 | 1.00 | 27.14 | O |
| ATOM | 4393 | CE2 | TYR | R | 36 | 29.726 | 93.558 | 156.891 | 1.00 | 28.23 | C |
| ATOM | 4395 | CD2 | TYR | R | 36 | 29.850 | 94.899 | 157.281 | 1.00 | 25.45 | C |
| ATOM | 4397 | C | TYR | R | 36 | 28.384 | 97.904 | 156.515 | 1.00 | 27.42 | C |
| ATOM | 4398 | O | TYR | R | 36 | 27.771 | 97.116 | 155.774 | 1.00 | 29.24 | O |
| ATOM | 4400 | N | PRO | R | 37 | 29.198 | 98.861 | 156.042 | 1.00 | 27.68 | N |
| ATOM | 4401 | CA | PRO | R | 37 | 29.415 | 99.039 | 154.620 | 1.00 | 28.95 | C |
| ATOM | 4403 | CB | PRO | R | 37 | 30.003 | 100.447 | 154.541 | 1.00 | 27.74 | C |
| ATOM | 4406 | CG | PRO | R | 37 | 30.698 | 100.626 | 155.827 | 1.00 | 28.32 | C |
| ATOM | 4409 | CD | PRO | R | 37 | 29.945 | 99.850 | 156.842 | 1.00 | 26.68 | C |
| ATOM | 4412 | C | PRO | R | 37 | 30.388 | 98.006 | 154.060 | 1.00 | 32.41 | C |
| ATOM | 4413 | O | PRO | R | 37 | 31.498 | 97.856 | 154.581 | 1.00 | 33.45 | O |
| ATOM | 4414 | N | SER | R | 38 | 29.969 | 97.298 | 153.014 | 1.00 | 30.58 | N |
| ATOM | 4415 | CA | SER | R | 38 | 30.844 | 96.344 | 152.340 | 1.00 | 35.75 | C |
| ATOM | 4417 | CB | SER | R | 38 | 30.987 | 95.070 | 153.185 | 1.00 | 39.20 | C |
| ATOM | 4420 | OG | SER | R | 38 | 31.989 | 94.209 | 152.671 | 1.00 | 39.12 | O |
| ATOM | 4422 | C | SER | R | 38 | 30.311 | 95.996 | 150.955 | 1.00 | 37.47 | C |
| ATOM | 4423 | O | SER | R | 38 | 29.096 | 95.911 | 150.755 | 1.00 | 34.52 | O |
| ATOM | 4425 | N | ASN | R | 39 | 31.221 | 95.817 | 149.998 | 1.00 | 40.27 | N |
| ATOM | 4426 | CA | ASN | R | 39 | 30.860 | 95.366 | 148.651 | 1.00 | 41.01 | C |
| ATOM | 4428 | CB | ASN | R | 39 | 30.486 | 93.875 | 148.686 | 1.00 | 44.45 | C |
| ATOM | 4431 | CG | ASN | R | 39 | 31.670 | 92.987 | 149.027 | 1.00 | 47.41 | C |
| ATOM | 4432 | OD1 | ASN | R | 39 | 32.530 | 92.735 | 148.182 | 1.00 | 51.95 | O |
| ATOM | 4433 | ND2 | ASN | R | 39 | 31.722 | 92.510 | 150.268 | 1.00 | 45.84 | N |
| ATOM | 4436 | C | ASN | R | 39 | 29.727 | 96.196 | 148.025 | 1.00 | 41.85 | C |
| ATOM | 4437 | O | ASN | R | 39 | 28.777 | 95.649 | 147.451 | 1.00 | 43.29 | O |
| ATOM | 4439 | N | ASP | R | 40 | 29.835 | 97.516 | 148.161 | 1.00 | 40.41 | N |
| ATOM | 4440 | CA | ASP | R | 40 | 28.860 | 98.475 | 147.615 | 1.00 | 41.55 | C |
| ATOM | 4442 | CB | ASP | R | 40 | 28.826 | 98.401 | 146.079 | 1.00 | 48.28 | C |
| ATOM | 4445 | CG | ASP | R | 40 | 30.151 | 98.785 | 145.438 | 1.00 | 53.53 | C |
| ATOM | 4446 | OD1 | ASP | R | 40 | 30.987 | 99.439 | 146.100 | 1.00 | 56.57 | O |
| ATOM | 4447 | OD2 | ASP | R | 40 | 30.348 | 98.444 | 144.251 | 1.00 | 59.35 | O |
| ATOM | 4448 | C | ASP | R | 40 | 27.436 | 98.356 | 148.174 | 1.00 | 38.17 | C |
| ATOM | 4449 | O | ASP | R | 40 | 26.471 | 98.685 | 147.488 | 1.00 | 38.74 | O |
| ATOM | 4451 | N | ARG | R | 41 | 27.302 | 97.904 | 149.417 | 1.00 | 37.16 | N |
| ATOM | 4452 | CA | ARG | R | 41 | 26.000 | 97.869 | 150.081 | 1.00 | 34.97 | C |
| ATOM | 4454 | CB | ARG | R | 41 | 25.182 | 96.659 | 149.612 | 1.00 | 37.69 | C |
| ATOM | 4457 | CG | ARG | R | 41 | 25.683 | 95.311 | 150.106 | 1.00 | 36.81 | C |
| ATOM | 4460 | CD | ARG | R | 41 | 24.910 | 94.184 | 149.448 | 1.00 | 38.16 | C |
| ATOM | 4463 | NE | ARG | R | 41 | 25.298 | 92.871 | 149.966 | 1.00 | 37.15 | N |
| ATOM | 4465 | CZ | ARG | R | 41 | 24.749 | 92.270 | 151.021 | 1.00 | 37.68 | C |
| ATOM | 4466 | NH1 | ARG | R | 41 | 23.776 | 92.849 | 151.717 | 1.00 | 36.39 | N |
| ATOM | 4469 | NH2 | ARG | R | 41 | 25.186 | 91.075 | 151.391 | 1.00 | 37.68 | N |
| ATOM | 4472 | C | ARG | R | 41 | 26.130 | 97.866 | 151.598 | 1.00 | 33.22 | C |
| ATOM | 4473 | O | ARG | R | 41 | 27.238 | 97.797 | 152.144 | 1.00 | 28.29 | O |
| ATOM | 4475 | N | CYS | R | 42 | 24.990 | 97.957 | 152.276 | 1.00 | 31.23 | N |
| ATOM | 4476 | CA | CYS | R | 42 | 24.958 | 97.939 | 153.731 | 1.00 | 33.69 | C |
| ATOM | 4478 | CB | CYS | R | 42 | 23.982 | 98.989 | 154.251 | 1.00 | 35.92 | C |
| ATOM | 4481 | SG | CYS | R | 42 | 24.452 | 100.661 | 153.756 | 1.00 | 38.34 | S |
| ATOM | 4483 | C | CYS | R | 42 | 24.582 | 96.551 | 154.229 | 1.00 | 33.48 | C |
| ATOM | 4484 | O | CYS | R | 42 | 23.430 | 96.146 | 154.149 | 1.00 | 31.75 | O |
| ATOM | 4486 | N | CYS | R | 43 | 25.571 | 95.821 | 154.734 | 1.00 | 32.23 | N |
| ATOM | 4487 | CA | CYS | R | 43 | 25.347 | 94.466 | 155.217 | 1.00 | 31.50 | C |
| ATOM | 4489 | CB | CYS | R | 43 | 26.625 | 93.643 | 155.087 | 1.00 | 33.21 | C |
| ATOM | 4492 | SG | CYS | R | 43 | 27.180 | 93.422 | 153.366 | 1.00 | 35.12 | S |
| ATOM | 4494 | C | CYS | R | 43 | 24.825 | 94.514 | 156.655 | 1.00 | 29.59 | C |
| ATOM | 4495 | O | CYS | R | 43 | 25.019 | 95.500 | 157.358 | 1.00 | 31.21 | O |
| ATOM | 4497 | N | HIS | R | 44 | 24.140 | 93.457 | 157.077 | 1.00 | 32.52 | N |
| ATOM | 4498 | CA | HIS | R | 44 | 23.461 | 93.440 | 158.368 | 1.00 | 31.47 | C |
| ATOM | 4500 | CB | HIS | R | 44 | 22.077 | 92.812 | 158.221 | 1.00 | 36.17 | C |
| ATOM | 4503 | CG | HIS | R | 44 | 21.191 | 93.552 | 157.268 | 1.00 | 36.62 | C |
| ATOM | 4504 | ND1 | HIS | R | 44 | 20.285 | 94.503 | 157.681 | 1.00 | 41.14 | N |
| ATOM | 4506 | CE1 | HIS | R | 44 | 19.660 | 94.999 | 156.628 | 1.00 | 39.90 | C |
| ATOM | 4508 | NE2 | HIS | R | 44 | 20.139 | 94.414 | 155.545 | 1.00 | 39.22 | N |
| ATOM | 4510 | CD2 | HIS | R | 44 | 21.102 | 93.509 | 155.918 | 1.00 | 38.43 | C |
| ATOM | 4512 | C | HIS | R | 44 | 24.267 | 92.713 | 159.434 | 1.00 | 28.73 | C |
| ATOM | 4513 | O | HIS | R | 44 | 24.907 | 91.709 | 159.162 | 1.00 | 31.01 | O |
| ATOM | 4515 | N | GLU | R | 45 | 24.237 | 93.245 | 160.649 | 1.00 | 31.29 | N |
| ATOM | 4516 | CA | GLU | R | 45 | 24.874 | 92.599 | 161.790 | 1.00 | 32.85 | C |
| ATOM | 4518 | CB | GLU | R | 45 | 25.197 | 93.627 | 162.862 | 1.00 | 34.07 | C |
| ATOM | 4521 | CG | GLU | R | 45 | 26.243 | 94.638 | 162.401 | 1.00 | 36.23 | C |
| ATOM | 4524 | CD | GLU | R | 45 | 26.469 | 95.761 | 163.388 | 1.00 | 38.00 | C |
| ATOM | 4525 | OE1 | GLU | R | 45 | 25.552 | 96.094 | 164.153 | 1.00 | 42.91 | O |
| ATOM | 4526 | OE2 | GLU | R | 45 | 27.570 | 96.332 | 163.386 | 1.00 | 49.67 | O |
| ATOM | 4527 | C | GLU | R | 45 | 23.931 | 91.523 | 162.311 | 1.00 | 32.49 | C |
| ATOM | 4528 | O | GLU | R | 45 | 22.776 | 91.466 | 161.902 | 1.00 | 33.12 | O |
| ATOM | 4530 | N | CYS | R | 46 | 24.433 | 90.655 | 163.183 | 1.00 | 30.35 | N |
| ATOM | 4531 | CA | CYS | R | 46 | 23.642 | 89.541 | 163.700 | 1.00 | 33.51 | C |
| ATOM | 4533 | CB | CYS | R | 46 | 24.561 | 88.392 | 164.120 | 1.00 | 34.70 | C |
| ATOM | 4536 | SG | CYS | R | 46 | 25.551 | 87.714 | 162.761 | 1.00 | 37.69 | S |
| ATOM | 4538 | C | CYS | R | 46 | 22.763 | 89.970 | 164.876 | 1.00 | 34.47 | C |
| ATOM | 4539 | O | CYS | R | 46 | 23.211 | 90.698 | 165.753 | 1.00 | 37.61 | O |
| ATOM | 4541 | N | ARG | R | 47 | 21.518 | 89.502 | 164.882 | 1.00 | 33.33 | N |
| ATOM | 4542 | CA | ARG | R | 47 | 20.566 | 89.806 | 165.947 | 1.00 | 36.56 | C |
| ATOM | 4544 | CB | ARG | R | 47 | 19.155 | 89.391 | 165.529 | 1.00 | 40.56 | C |
| ATOM | 4547 | CG | ARG | R | 47 | 18.625 | 90.019 | 164.241 | 1.00 | 45.79 | C |
| ATOM | 4550 | CD | ARG | R | 47 | 17.216 | 89.491 | 163.939 | 1.00 | 49.44 | C |
| ATOM | 4553 | NE | ARG | R | 47 | 17.026 | 89.162 | 162.526 | 1.00 | 53.43 | N |
| ATOM | 4555 | CZ | ARG | R | 47 | 16.643 | 90.018 | 161.579 | 1.00 | 56.07 | C |
| ATOM | 4556 | NH1 | ARG | R | 47 | 16.391 | 91.295 | 161.861 | 1.00 | 57.99 | N |
| ATOM | 4559 | NH2 | ARG | R | 47 | 16.512 | 89.591 | 160.326 | 1.00 | 57.41 | N |
| ATOM | 4562 | C | ARG | R | 47 | 20.920 | 89.072 | 167.248 | 1.00 | 34.49 | C |
| ATOM | 4563 | O | ARG | R | 47 | 21.683 | 88.102 | 167.236 | 1.00 | 31.54 | O |
| ATOM | 4565 | N | PRO | R | 48 | 20.348 | 89.522 | 168.381 | 1.00 | 35.95 | N |
| ATOM | 4566 | CA | PRO | R | 48 | 20.495 | 88.774 | 169.630 | 1.00 | 33.16 | C |
| ATOM | 4568 | CB | PRO | R | 48 | 19.507 | 89.478 | 170.568 | 1.00 | 34.82 | C |
| ATOM | 4571 | CG | PRO | R | 48 | 19.479 | 90.886 | 170.064 | 1.00 | 35.62 | C |
| ATOM | 4574 | CD | PRO | R | 48 | 19.551 | 90.752 | 168.574 | 1.00 | 35.51 | C |
| ATOM | 4577 | C | PRO | R | 48 | 20.138 | 87.298 | 169.466 | 1.00 | 30.90 | C |
| ATOM | 4578 | O | PRO | R | 48 | 19.191 | 86.971 | 168.751 | 1.00 | 30.07 | O |
| ATOM | 4579 | N | GLY | R | 49 | 20.893 | 86.420 | 170.119 | 1.00 | 28.21 | N |
| ATOM | 4580 | CA | GLY | R | 49 | 20.691 | 84.978 | 169.995 | 1.00 | 29.71 | C |
| ATOM | 4583 | C | GLY | R | 49 | 21.464 | 84.319 | 168.867 | 1.00 | 30.51 | C |
| ATOM | 4584 | O | GLY | R | 49 | 21.421 | 83.094 | 168.713 | 1.00 | 29.20 | O |
| ATOM | 4586 | N | ASN | R | 50 | 22.143 | 85.141 | 168.070 | 1.00 | 30.48 | N |
| ATOM | 4587 | CA | ASN | R | 50 | 23.013 | 84.698 | 166.987 | 1.00 | 29.74 | C |
| ATOM | 4589 | CB | ASN | R | 50 | 22.533 | 85.232 | 165.630 | 1.00 | 29.26 | C |
| ATOM | 4592 | CG | ASN | R | 50 | 21.296 | 84.539 | 165.102 | 1.00 | 28.97 | C |
| ATOM | 4593 | OD1 | ASN | R | 50 | 21.000 | 83.389 | 165.431 | 1.00 | 28.47 | O |
| ATOM | 4594 | ND2 | ASN | R | 50 | 20.567 | 85.247 | 164.235 | 1.00 | 29.84 | N |
| ATOM | 4597 | C | ASN | R | 50 | 24.416 | 85.239 | 167.207 | 1.00 | 30.15 | C |
| ATOM | 4598 | O | ASN | R | 50 | 24.603 | 86.270 | 167.872 | 1.00 | 30.74 | O |
| ATOM | 4600 | N | GLY | R | 51 | 25.395 | 84.544 | 166.630 | 1.00 | 30.31 | N |
| ATOM | 4601 | CA | GLY | R | 51 | 26.769 | 85.018 | 166.555 | 1.00 | 29.72 | C |
| ATOM | 4604 | C | GLY | R | 51 | 27.240 | 85.102 | 165.115 | 1.00 | 29.68 | C |
| ATOM | 4605 | O | GLY | R | 51 | 26.781 | 84.344 | 164.259 | 1.00 | 29.73 | O |
| ATOM | 4607 | N | MET | R | 52 | 28.157 | 86.026 | 164.828 | 1.00 | 29.58 | N |
| ATOM | 4608 | CA | MET | R | 52 | 28.641 | 86.191 | 163.456 | 1.00 | 31.58 | C |
| ATOM | 4610 | CB | MET | R | 52 | 29.138 | 87.618 | 163.166 | 1.00 | 32.45 | C |
| ATOM | 4613 | CG | MET | R | 52 | 29.282 | 87.921 | 161.627 | 1.00 | 31.49 | C |
| ATOM | 4616 | SD | MET | R | 52 | 29.766 | 89.608 | 161.254 | 1.00 | 38.14 | S |
| ATOM | 4617 | CE | MET | R | 52 | 28.199 | 90.458 | 161.291 | 1.00 | 36.96 | C |
| ATOM | 4621 | C | MET | R | 52 | 29.738 | 85.176 | 163.150 | 1.00 | 31.29 | C |
| ATOM | 4622 | O | MET | R | 52 | 30.729 | 85.044 | 163.888 | 1.00 | 30.38 | O |
| ATOM | 4624 | N | VAL | R | 53 | 29.534 | 84.443 | 162.062 | 1.00 | 30.33 | N |
| ATOM | 4625 | CA | VAL | R | 53 | 30.502 | 83.461 | 161.584 | 1.00 | 28.02 | C |
| ATOM | 4627 | CB | VAL | R | 53 | 29.782 | 82.200 | 161.056 | 1.00 | 27.53 | C |
| ATOM | 4629 | CG1 | VAL | R | 53 | 30.769 | 81.189 | 160.484 | 1.00 | 30.81 | C |
| ATOM | 4633 | CG2 | VAL | R | 53 | 28.948 | 81.562 | 162.171 | 1.00 | 31.04 | C |
| ATOM | 4637 | C | VAL | R | 53 | 31.373 | 84.102 | 160.504 | 1.00 | 27.05 | C |
| ATOM | 4638 | O | VAL | R | 53 | 32.594 | 83.917 | 160.480 | 1.00 | 27.90 | O |
| ATOM | 4640 | N | SER | R | 54 | 30.750 | 84.861 | 159.608 | 1.00 | 27.21 | N |
| ATOM | 4641 | CA | SER | R | 54 | 31.496 | 85.538 | 158.547 | 1.00 | 25.29 | C |
| ATOM | 4643 | CB | SER | R | 54 | 31.765 | 84.587 | 157.376 | 1.00 | 27.92 | C |
| ATOM | 4646 | OG | SER | R | 54 | 30.563 | 84.038 | 156.859 | 1.00 | 28.05 | O |
| ATOM | 4648 | C | SER | R | 54 | 30.764 | 86.763 | 158.045 | 1.00 | 28.58 | C |
| ATOM | 4649 | O | SER | R | 54 | 29.541 | 86.755 | 157.910 | 1.00 | 28.69 | O |
| ATOM | 4651 | N | ARG | R | 55 | 31.521 | 87.818 | 157.761 | 1.00 | 29.09 | N |
| ATOM | 4652 | CA | ARG | R | 55 | 30.963 | 89.014 | 157.143 | 1.00 | 26.28 | C |
| ATOM | 4654 | CB | ARG | R | 55 | 31.944 | 90.172 | 157.236 | 1.00 | 27.77 | C |
| ATOM | 4657 | CG | ARG | R | 55 | 32.141 | 90.668 | 158.657 | 1.00 | 30.02 | C |
| ATOM | 4660 | CD | ARG | R | 55 | 32.594 | 92.065 | 158.635 | 1.00 | 28.05 | C |
| ATOM | 4663 | NE | ARG | R | 55 | 32.967 | 92.588 | 159.941 | 1.00 | 29.72 | N |
| ATOM | 4665 | CZ | ARG | R | 55 | 32.216 | 93.380 | 160.705 | 1.00 | 27.14 | C |
| ATOM | 4666 | NH1 | ARG | R | 55 | 30.988 | 93.720 | 160.354 | 1.00 | 28.65 | N |
| ATOM | 4669 | NH2 | ARG | R | 55 | 32.703 | 93.824 | 161.854 | 1.00 | 25.33 | N |
| ATOM | 4672 | C | ARG | R | 55 | 30.604 | 88.741 | 155.697 | 1.00 | 29.87 | C |
| ATOM | 4673 | O | ARG | R | 55 | 31.092 | 87.781 | 155.100 | 1.00 | 31.83 | O |
| ATOM | 4675 | N | CYS | R | 56 | 29.728 | 89.577 | 155.144 | 1.00 | 29.35 | N |
| ATOM | 4676 | CA | CYS | R | 56 | 29.326 | 89.455 | 153.755 | 1.00 | 29.68 | C |
| ATOM | 4678 | CB | CYS | R | 56 | 28.263 | 90.513 | 153.389 | 1.00 | 31.88 | C |
| ATOM | 4681 | SG | CYS | R | 56 | 28.858 | 92.233 | 153.470 | 1.00 | 37.15 | S |
| ATOM | 4683 | C | CYS | R | 56 | 30.555 | 89.575 | 152.853 | 1.00 | 32.70 | C |
| ATOM | 4684 | O | CYS | R | 56 | 31.551 | 90.218 | 153.207 | 1.00 | 30.66 | O |
| ATOM | 4686 | N | SER | R | 57 | 30.497 | 88.907 | 151.709 | 1.00 | 30.96 | N |
| ATOM | 4687 | CA | SER | R | 57 | 31.517 | 89.043 | 150.677 | 1.00 | 33.44 | C |
| ATOM | 4689 | CB | SER | R | 57 | 32.301 | 87.740 | 150.532 | 1.00 | 33.76 | C |
| ATOM | 4692 | OG | SER | R | 57 | 31.528 | 86.758 | 149.855 | 1.00 | 32.09 | O |
| ATOM | 4694 | C | SER | R | 57 | 30.812 | 89.396 | 149.369 | 1.00 | 35.67 | C |
| ATOM | 4695 | O | SER | R | 57 | 29.593 | 89.605 | 149.341 | 1.00 | 34.42 | O |
| ATOM | 4697 | N | ARG | R | 58 | 31.577 | 89.474 | 148.290 | 1.00 | 34.86 | N |
| ATOM | 4698 | CA | ARG | R | 58 | 31.010 | 89.749 | 146.975 | 1.00 | 37.51 | C |
| ATOM | 4700 | CB | ARG | R | 58 | 32.118 | 89.970 | 145.933 | 1.00 | 43.12 | C |
| ATOM | 4703 | CG | ARG | R | 58 | 33.115 | 88.819 | 145.791 | 1.00 | 49.66 | C |
| ATOM | 4706 | CD | ARG | R | 58 | 34.555 | 89.314 | 145.917 | 1.00 | 55.38 | C |
| ATOM | 4709 | NE | ARG | R | 58 | 34.797 | 89.923 | 147.231 | 1.00 | 58.71 | N |
| ATOM | 4711 | CZ | ARG | R | 58 | 35.092 | 89.260 | 148.350 | 1.00 | 56.44 | C |
| ATOM | 4712 | NH1 | ARG | R | 58 | 35.201 | 87.933 | 148.352 | 1.00 | 57.41 | N |
| ATOM | 4715 | NH2 | ARG | R | 58 | 35.284 | 89.934 | 149.482 | 1.00 | 55.87 | N |
| ATOM | 4718 | C | ARG | R | 58 | 30.041 | 88.648 | 146.523 | 1.00 | 35.92 | C |
| ATOM | 4719 | O | ARG | R | 58 | 29.094 | 88.929 | 145.809 | 1.00 | 36.92 | O |
| ATOM | 4721 | N | SER | R | 59 | 30.260 | 87.411 | 146.966 | 1.00 | 33.51 | N |
| ATOM | 4722 | CA | SER | R | 59 | 29.434 | 86.287 | 146.527 | 1.00 | 36.40 | C |
| ATOM | 4724 | CB | SER | R | 59 | 30.325 | 85.191 | 145.944 | 1.00 | 38.81 | C |
| ATOM | 4727 | OG | SER | R | 59 | 31.178 | 84.651 | 146.931 | 1.00 | 39.42 | O |
| ATOM | 4729 | C | SER | R | 59 | 28.538 | 85.682 | 147.621 | 1.00 | 33.02 | C |
| ATOM | 4730 | O | SER | R | 59 | 27.709 | 84.828 | 147.322 | 1.00 | 32.07 | O |
| ATOM | 4732 | N | GLN | R | 60 | 28.689 | 86.127 | 148.866 | 1.00 | 32.27 | N |
| ATOM | 4733 | CA | GLN | R | 60 | 27.994 | 85.492 | 149.993 | 1.00 | 32.20 | C |
| ATOM | 4735 | CB | GLN | R | 60 | 28.963 | 84.534 | 150.687 | 1.00 | 33.10 | C |
| ATOM | 4738 | CG | GLN | R | 60 | 28.325 | 83.506 | 151.575 | 1.00 | 35.02 | C |
| ATOM | 4741 | CD | GLN | R | 60 | 29.343 | 82.551 | 152.165 | 1.00 | 35.93 | C |
| ATOM | 4742 | OE1 | GLN | R | 60 | 29.443 | 81.401 | 151.742 | 1.00 | 40.27 | O |
| ATOM | 4743 | NE2 | GLN | R | 60 | 30.110 | 83.027 | 153.141 | 1.00 | 29.84 | N |
| ATOM | 4746 | C | GLN | R | 60 | 27.458 | 86.519 | 150.990 | 1.00 | 31.11 | C |
| ATOM | 4747 | O | GLN | R | 60 | 28.160 | 87.468 | 151.343 | 1.00 | 30.64 | O |
| ATOM | 4749 | N | ASN | R | 61 | 26.218 | 86.325 | 151.448 | 1.00 | 28.33 | N |
| ATOM | 4750 | CA | ASN | R | 61 | 25.674 | 87.112 | 152.571 | 1.00 | 28.02 | C |
| ATOM | 4752 | CB | ASN | R | 61 | 24.195 | 86.779 | 152.814 | 1.00 | 25.78 | C |
| ATOM | 4755 | CG | ASN | R | 61 | 23.267 | 87.323 | 151.728 | 1.00 | 26.40 | C |
| ATOM | 4756 | OD1 | ASN | R | 61 | 23.635 | 88.194 | 150.949 | 1.00 | 29.07 | O |
| ATOM | 4757 | ND2 | ASN | R | 61 | 22.051 | 86.819 | 151.697 | 1.00 | 25.95 | N |
| ATOM | 4760 | C | ASN | R | 61 | 26.440 | 86.861 | 153.869 | 1.00 | 29.13 | C |
| ATOM | 4761 | O | ASN | R | 61 | 27.146 | 85.864 | 154.003 | 1.00 | 29.13 | O |
| ATOM | 4763 | N | THR | R | 62 | 26.289 | 87.781 | 154.822 | 1.00 | 27.26 | N |
| ATOM | 4764 | CA | THR | R | 62 | 26.685 | 87.562 | 156.205 | 1.00 | 29.13 | C |
| ATOM | 4766 | CB | THR | R | 62 | 26.100 | 88.659 | 157.118 | 1.00 | 32.11 | C |
| ATOM | 4768 | OG1 | THR | R | 62 | 26.481 | 89.946 | 156.627 | 1.00 | 31.13 | O |
| ATOM | 4770 | CG2 | THR | R | 62 | 26.574 | 88.497 | 158.555 | 1.00 | 31.30 | C |
| ATOM | 4774 | C | THR | R | 62 | 26.145 | 86.224 | 156.693 | 1.00 | 28.13 | C |
| ATOM | 4775 | O | THR | R | 62 | 24.999 | 85.877 | 156.407 | 1.00 | 26.55 | O |
| ATOM | 4777 | N | VAL | R | 63 | 26.969 | 85.479 | 157.418 | 1.00 | 28.36 | N |
| ATOM | 4778 | CA | VAL | R | 63 | 26.536 | 84.225 | 158.037 | 1.00 | 28.90 | C |
| ATOM | 4780 | CB | VAL | R | 63 | 27.490 | 83.053 | 157.700 | 1.00 | 29.70 | C |
| ATOM | 4782 | CG1 | VAL | R | 63 | 27.002 | 81.758 | 158.346 | 1.00 | 30.27 | C |
| ATOM | 4786 | CG2 | VAL | R | 63 | 27.622 | 82.901 | 156.207 | 1.00 | 31.20 | C |
| ATOM | 4790 | C | VAL | R | 63 | 26.476 | 84.395 | 159.549 | 1.00 | 29.89 | C |
| ATOM | 4791 | O | VAL | R | 63 | 27.499 | 84.628 | 160.196 | 1.00 | 26.01 | O |
| ATOM | 4793 | N | CYS | R | 64 | 25.266 | 84.277 | 160.091 | 1.00 | 29.40 | N |
| ATOM | 4794 | CA | CYS | R | 64 | 24.992 | 84.305 | 161.525 | 1.00 | 33.15 | C |
| ATOM | 4796 | CB | CYS | R | 64 | 23.872 | 85.307 | 161.844 | 1.00 | 34.63 | C |
| ATOM | 4799 | SG | CYS | R | 64 | 24.209 | 87.037 | 161.387 | 1.00 | 38.55 | S |
| ATOM | 4801 | C | CYS | R | 64 | 24.535 | 82.905 | 161.931 | 1.00 | 31.30 | C |
| ATOM | 4802 | O | CYS | R | 64 | 23.837 | 82.231 | 161.166 | 1.00 | 30.68 | O |
| ATOM | 4804 | N | ARG | R | 65 | 24.921 | 82.468 | 163.123 | 1.00 | 32.20 | N |
| ATOM | 4805 | CA | ARG | R | 65 | 24.578 | 81.126 | 163.612 | 1.00 | 34.89 | C |
| ATOM | 4807 | CB | ARG | R | 65 | 25.832 | 80.256 | 163.648 | 1.00 | 39.55 | C |
| ATOM | 4810 | CG | ARG | R | 65 | 25.607 | 78.811 | 164.036 | 1.00 | 42.23 | C |
| ATOM | 4813 | CD | ARG | R | 65 | 26.917 | 78.014 | 164.131 | 1.00 | 48.78 | C |
| ATOM | 4816 | NE | ARG | R | 65 | 26.647 | 76.566 | 164.149 | 1.00 | 52.41 | N |
| ATOM | 4818 | CZ | ARG | R | 65 | 26.874 | 75.710 | 163.147 | 1.00 | 54.03 | C |
| ATOM | 4819 | NH1 | ARG | R | 65 | 27.420 | 76.104 | 161.997 | 1.00 | 56.08 | N |
| ATOM | 4822 | NH2 | ARG | R | 65 | 26.568 | 74.426 | 163.307 | 1.00 | 55.34 | N |
| ATOM | 4825 | C | ARG | R | 65 | 23.982 | 81.252 | 165.012 | 1.00 | 30.86 | C |
| ATOM | 4826 | O | ARG | R | 65 | 24.441 | 82.085 | 165.794 | 1.00 | 31.67 | O |
| ATOM | 4828 | N | PRO | R | 66 | 22.986 | 80.416 | 165.351 | 1.00 | 28.23 | N |
| ATOM | 4829 | CA | PRO | R | 66 | 22.412 | 80.507 | 166.697 | 1.00 | 31.49 | C |
| ATOM | 4831 | CB | PRO | R | 66 | 21.349 | 79.405 | 166.717 | 1.00 | 30.68 | C |
| ATOM | 4834 | CG | PRO | R | 66 | 21.157 | 78.988 | 165.344 | 1.00 | 31.07 | C |
| ATOM | 4837 | CD | PRO | R | 66 | 22.357 | 79.347 | 164.553 | 1.00 | 28.62 | C |
| ATOM | 4840 | C | PRO | R | 66 | 23.460 | 80.216 | 167.755 | 1.00 | 30.63 | C |
| ATOM | 4841 | O | PRO | R | 66 | 24.316 | 79.371 | 167.533 | 1.00 | 32.34 | O |
| ATOM | 4842 | N | CYS | R | 67 | 23.387 | 80.895 | 168.896 | 1.00 | 32.88 | N |
| ATOM | 4843 | CA | CYS | R | 67 | 24.317 | 80.630 | 169.985 | 1.00 | 34.81 | C |
| ATOM | 4845 | CB | CYS | R | 67 | 24.110 | 81.610 | 171.140 | 1.00 | 34.33 | C |
| ATOM | 4848 | SG | CYS | R | 67 | 24.385 | 83.324 | 170.714 | 1.00 | 36.42 | S |
| ATOM | 4850 | C | CYS | R | 67 | 24.152 | 79.191 | 170.470 | 1.00 | 34.59 | C |
| ATOM | 4851 | O | CYS | R | 67 | 23.033 | 78.718 | 170.680 | 1.00 | 36.19 | O |
| ATOM | 4853 | N | GLY | R | 68 | 25.274 | 78.499 | 170.625 | 1.00 | 36.01 | N |
| ATOM | 4854 | CA | GLY | R | 68 | 25.265 | 77.120 | 171.099 | 1.00 | 38.18 | C |
| ATOM | 4857 | C | GLY | R | 68 | 25.040 | 77.027 | 172.595 | 1.00 | 38.55 | C |
| ATOM | 4858 | O | GLY | R | 68 | 24.996 | 78.052 | 173.287 | 1.00 | 35.06 | O |
| ATOM | 4860 | N | PRO | R | 69 | 24.893 | 75.793 | 173.111 | 1.00 | 40.65 | N |
| ATOM | 4861 | CA | PRO | R | 69 | 24.681 | 75.597 | 174.547 | 1.00 | 39.03 | C |
| ATOM | 4863 | CB | PRO | R | 69 | 24.688 | 74.070 | 174.704 | 1.00 | 41.74 | C |
| ATOM | 4866 | CG | PRO | R | 69 | 24.354 | 73.537 | 173.355 | 1.00 | 43.58 | C |
| ATOM | 4869 | CD | PRO | R | 69 | 24.910 | 74.516 | 172.371 | 1.00 | 43.21 | C |
| ATOM | 4872 | C | PRO | R | 69 | 25.796 | 76.228 | 175.373 | 1.00 | 33.91 | C |
| ATOM | 4873 | O | PRO | R | 69 | 26.974 | 76.038 | 175.066 | 1.00 | 33.40 | O |
| ATOM | 4874 | N | GLY | R | 70 | 25.423 | 76.991 | 176.391 | 1.00 | 35.04 | N |
| ATOM | 4875 | CA | GLY | R | 70 | 26.398 | 77.686 | 177.239 | 1.00 | 35.67 | C |
| ATOM | 4878 | C | GLY | R | 70 | 26.903 | 79.011 | 176.688 | 1.00 | 31.97 | C |
| ATOM | 4879 | O | GLY | R | 70 | 27.821 | 79.601 | 177.253 | 1.00 | 31.94 | O |
| ATOM | 4881 | N | PHE | R | 71 | 26.297 | 79.494 | 175.602 | 1.00 | 31.75 | N |
| ATOM | 4882 | CA | PHE | R | 71 | 26.686 | 80.767 | 174.986 | 1.00 | 33.00 | C |
| ATOM | 4884 | CB | PHE | R | 71 | 27.453 | 80.537 | 173.674 | 1.00 | 34.75 | C |
| ATOM | 4887 | CG | PHE | R | 71 | 28.827 | 79.957 | 173.865 | 1.00 | 33.67 | C |
| ATOM | 4888 | CD1 | PHE | R | 71 | 28.991 | 78.607 | 174.131 | 1.00 | 36.06 | C |
| ATOM | 4890 | CE1 | PHE | R | 71 | 30.253 | 78.068 | 174.326 | 1.00 | 38.06 | C |
| ATOM | 4892 | CZ | PHE | R | 71 | 31.375 | 78.884 | 174.243 | 1.00 | 38.56 | C |
| ATOM | 4894 | CE2 | PHE | R | 71 | 31.226 | 80.233 | 173.971 | 1.00 | 36.77 | C |
| ATOM | 4896 | CD2 | PHE | R | 71 | 29.956 | 80.761 | 173.782 | 1.00 | 36.48 | C |
| ATOM | 4898 | C | PHE | R | 71 | 25.468 | 81.641 | 174.718 | 1.00 | 33.47 | C |
| ATOM | 4899 | O | PHE | R | 71 | 24.358 | 81.140 | 174.535 | 1.00 | 32.23 | O |
| ATOM | 4901 | N | TYR | R | 72 | 25.691 | 82.952 | 174.691 | 1.00 | 30.53 | N |
| ATOM | 4902 | CA | TYR | R | 72 | 24.621 | 83.925 | 174.509 | 1.00 | 32.63 | C |
| ATOM | 4904 | CB | TYR | R | 72 | 24.071 | 84.381 | 175.873 | 1.00 | 33.63 | C |
| ATOM | 4907 | CG | TYR | R | 72 | 24.929 | 85.427 | 176.533 | 1.00 | 32.43 | C |
| ATOM | 4908 | CD1 | TYR | R | 72 | 26.109 | 85.080 | 177.187 | 1.00 | 32.28 | C |
| ATOM | 4910 | CE1 | TYR | R | 72 | 26.907 | 86.050 | 177.780 | 1.00 | 35.41 | C |
| ATOM | 4912 | CZ | TYR | R | 72 | 26.532 | 87.375 | 177.707 | 1.00 | 33.52 | C |
| ATOM | 4913 | OH | TYR | R | 72 | 27.311 | 88.359 | 178.273 | 1.00 | 35.08 | O |
| ATOM | 4915 | CE2 | TYR | R | 72 | 25.374 | 87.740 | 177.064 | 1.00 | 35.41 | C |
| ATOM | 4917 | CD2 | TYR | R | 72 | 24.574 | 86.773 | 176.487 | 1.00 | 32.63 | C |
| ATOM | 4919 | C | TYR | R | 72 | 25.100 | 85.143 | 173.725 | 1.00 | 31.76 | C |
| ATOM | 4920 | O | TYR | R | 72 | 26.306 | 85.386 | 173.577 | 1.00 | 30.90 | O |
| ATOM | 4922 | N | ASN | R | 73 | 24.130 | 85.884 | 173.208 | 1.00 | 35.11 | N |
| ATOM | 4923 | CA | ASN | R | 73 | 24.365 | 87.176 | 172.593 | 1.00 | 34.31 | C |
| ATOM | 4925 | CB | ASN | R | 73 | 24.593 | 87.063 | 171.086 | 1.00 | 36.39 | C |
| ATOM | 4928 | CG | ASN | R | 73 | 24.882 | 88.413 | 170.448 | 1.00 | 43.42 | C |
| ATOM | 4929 | OD1 | ASN | R | 73 | 25.695 | 89.175 | 170.977 | 1.00 | 53.93 | O |
| ATOM | 4930 | ND2 | ASN | R | 73 | 24.217 | 88.727 | 169.313 | 1.00 | 41.04 | N |
| ATOM | 4933 | C | ASN | R | 73 | 23.159 | 88.047 | 172.845 | 1.00 | 31.14 | C |
| ATOM | 4934 | O | ASN | R | 73 | 22.094 | 87.808 | 172.272 | 1.00 | 31.98 | O |
| ATOM | 4936 | N | ASP | R | 74 | 23.336 | 89.056 | 173.695 | 1.00 | 31.37 | N |
| ATOM | 4937 | CA | ASP | R | 74 | 22.240 | 89.886 | 174.168 | 1.00 | 35.31 | C |
| ATOM | 4939 | CB | ASP | R | 74 | 22.430 | 90.245 | 175.658 | 1.00 | 38.09 | C |
| ATOM | 4942 | CG | ASP | R | 74 | 23.701 | 91.049 | 175.936 | 1.00 | 40.36 | C |
| ATOM | 4943 | OD1 | ASP | R | 74 | 24.747 | 90.846 | 175.282 | 1.00 | 41.90 | O |
| ATOM | 4944 | OD2 | ASP | R | 74 | 23.652 | 91.885 | 176.850 | 1.00 | 47.32 | O |
| ATOM | 4945 | C | ASP | R | 74 | 21.972 | 91.143 | 173.336 | 1.00 | 35.25 | C |
| ATOM | 4946 | O | ASP | R | 74 | 20.925 | 91.767 | 173.495 | 1.00 | 35.55 | O |
| ATOM | 4948 | N | VAL | R | 75 | 22.892 | 91.505 | 172.447 | 1.00 | 33.35 | N |
| ATOM | 4949 | CA | VAL | R | 75 | 22.710 | 92.683 | 171.591 | 1.00 | 37.93 | C |
| ATOM | 4951 | CB | VAL | R | 75 | 23.590 | 93.873 | 172.069 | 1.00 | 41.35 | C |
| ATOM | 4953 | CG1 | VAL | R | 75 | 23.129 | 94.357 | 173.430 | 1.00 | 44.63 | C |
| ATOM | 4957 | CG2 | VAL | R | 75 | 25.061 | 93.484 | 172.110 | 1.00 | 42.94 | C |
| ATOM | 4961 | C | VAL | R | 75 | 23.044 | 92.385 | 170.135 | 1.00 | 36.68 | C |
| ATOM | 4962 | O | VAL | R | 75 | 23.574 | 91.328 | 169.813 | 1.00 | 38.00 | O |
| ATOM | 4964 | N | VAL | R | 76 | 22.739 | 93.333 | 169.256 | 1.00 | 37.93 | N |
| ATOM | 4965 | CA | VAL | R | 76 | 23.156 | 93.235 | 167.858 | 1.00 | 39.65 | C |
| ATOM | 4967 | CB | VAL | R | 76 | 22.598 | 94.412 | 167.027 | 1.00 | 40.06 | C |
| ATOM | 4969 | CG1 | VAL | R | 76 | 23.256 | 94.493 | 165.672 | 1.00 | 41.55 | C |
| ATOM | 4973 | CG2 | VAL | R | 76 | 21.102 | 94.265 | 166.872 | 1.00 | 38.76 | C |
| ATOM | 4977 | C | VAL | R | 76 | 24.683 | 93.208 | 167.839 | 1.00 | 39.10 | C |
| ATOM | 4978 | O | VAL | R | 76 | 25.319 | 93.976 | 168.553 | 1.00 | 38.21 | O |
| ATOM | 4980 | N | SER | R | 77 | 25.268 | 92.320 | 167.034 | 1.00 | 39.48 | N |
| ATOM | 4981 | CA | SER | R | 77 | 26.702 | 92.028 | 167.127 | 1.00 | 38.80 | C |
| ATOM | 4983 | CB | SER | R | 77 | 26.912 | 90.898 | 168.144 | 1.00 | 43.02 | C |
| ATOM | 4986 | OG | SER | R | 77 | 28.226 | 90.383 | 168.092 | 1.00 | 45.90 | O |
| ATOM | 4988 | C | SER | R | 77 | 27.355 | 91.626 | 165.803 | 1.00 | 36.62 | C |
| ATOM | 4989 | O | SER | R | 77 | 26.701 | 91.090 | 164.906 | 1.00 | 34.04 | O |
| ATOM | 4991 | N | SER | R | 78 | 28.659 | 91.875 | 165.713 | 1.00 | 31.97 | N |
| ATOM | 4992 | CA | SER | R | 78 | 29.508 | 91.392 | 164.626 | 1.00 | 35.38 | C |
| ATOM | 4994 | CB | SER | R | 78 | 30.191 | 92.572 | 163.927 | 1.00 | 40.76 | C |
| ATOM | 4997 | OG | SER | R | 78 | 29.260 | 93.326 | 163.181 | 1.00 | 46.19 | O |
| ATOM | 4999 | C | SER | R | 78 | 30.568 | 90.430 | 165.166 | 1.00 | 33.73 | C |
| ATOM | 5000 | O | SER | R | 78 | 31.628 | 90.260 | 164.563 | 1.00 | 35.83 | O |
| ATOM | 5002 | N | LYS | R | 79 | 30.275 | 89.793 | 166.294 | 1.00 | 36.23 | N |
| ATOM | 5003 | CA | LYS | R | 79 | 31.211 | 88.882 | 166.936 | 1.00 | 37.39 | C |
| ATOM | 5005 | CB | LYS | R | 79 | 31.814 | 89.533 | 168.194 | 1.00 | 39.80 | C |
| ATOM | 5008 | CG | LYS | R | 79 | 32.760 | 90.692 | 167.882 | 1.00 | 41.39 | C |
| ATOM | 5011 | CD | LYS | R | 79 | 33.318 | 91.357 | 169.147 | 1.00 | 43.07 | C |
| ATOM | 5014 | CE | LYS | R | 79 | 34.464 | 92.318 | 168.818 | 1.00 | 41.96 | C |
| ATOM | 5017 | NZ | LYS | R | 79 | 34.897 | 93.210 | 169.954 | 1.00 | 42.37 | N |
| ATOM | 5021 | C | LYS | R | 79 | 30.531 | 87.562 | 167.280 | 1.00 | 34.11 | C |
| ATOM | 5022 | O | LYS | R | 79 | 29.305 | 87.479 | 167.298 | 1.00 | 33.57 | O |
| ATOM | 5024 | N | PRO | R | 80 | 31.331 | 86.509 | 167.527 | 1.00 | 35.25 | N |
| ATOM | 5025 | CA | PRO | R | 80 | 30.776 | 85.246 | 168.010 | 1.00 | 36.23 | C |
| ATOM | 5027 | CB | PRO | R | 80 | 32.015 | 84.373 | 168.213 | 1.00 | 37.14 | C |
| ATOM | 5030 | CG | PRO | R | 80 | 33.055 | 84.974 | 167.328 | 1.00 | 39.47 | C |
| ATOM | 5033 | CD | PRO | R | 80 | 32.792 | 86.428 | 167.330 | 1.00 | 37.86 | C |
| ATOM | 5036 | C | PRO | R | 80 | 30.024 | 85.418 | 169.333 | 1.00 | 33.74 | C |
| ATOM | 5037 | O | PRO | R | 80 | 30.226 | 86.405 | 170.033 | 1.00 | 30.78 | O |
| ATOM | 5038 | N | CYS | R | 81 | 29.159 | 84.463 | 169.649 | 1.00 | 33.59 | N |
| ATOM | 5039 | CA | CYS | R | 81 | 28.438 | 84.443 | 170.923 | 1.00 | 35.45 | C |
| ATOM | 5041 | CB | CYS | R | 81 | 27.456 | 83.273 | 170.957 | 1.00 | 37.81 | C |
| ATOM | 5044 | SG | CYS | R | 81 | 26.159 | 83.356 | 169.692 | 1.00 | 39.04 | S |
| ATOM | 5046 | C | CYS | R | 81 | 29.421 | 84.341 | 172.088 | 1.00 | 38.06 | C |
| ATOM | 5047 | O | CYS | R | 81 | 30.541 | 83.870 | 171.912 | 1.00 | 38.03 | O |
| ATOM | 5049 | N | LYS | R | 82 | 28.997 | 84.811 | 173.264 | 1.00 | 34.71 | N |
| ATOM | 5050 | CA | LYS | R | 82 | 29.849 | 84.883 | 174.450 | 1.00 | 34.60 | C |
| ATOM | 5052 | CB | LYS | R | 82 | 29.660 | 86.228 | 175.148 | 1.00 | 36.54 | C |
| ATOM | 5055 | CG | LYS | R | 82 | 29.931 | 87.434 | 174.278 | 1.00 | 39.68 | C |
| ATOM | 5058 | CD | LYS | R | 82 | 29.614 | 88.730 | 175.017 | 1.00 | 43.55 | C |
| ATOM | 5061 | CE | LYS | R | 82 | 28.131 | 89.052 | 174.964 | 1.00 | 43.86 | C |
| ATOM | 5064 | NZ | LYS | R | 82 | 27.823 | 90.344 | 175.657 | 1.00 | 42.03 | N |
| ATOM | 5068 | C | LYS | R | 82 | 29.505 | 83.765 | 175.434 | 1.00 | 33.15 | C |
| ATOM | 5069 | O | LYS | R | 82 | 28.357 | 83.357 | 175.520 | 1.00 | 30.25 | O |
| ATOM | 5071 | N | PRO | R | 83 | 30.495 | 83.277 | 176.196 | 1.00 | 34.94 | N |
| ATOM | 5072 | CA | PRO | R | 83 | 30.161 | 82.222 | 177.138 | 1.00 | 33.35 | C |
| ATOM | 5074 | CB | PRO | R | 83 | 31.522 | 81.699 | 177.606 | 1.00 | 37.58 | C |
| ATOM | 5077 | CG | PRO | R | 83 | 32.521 | 82.701 | 177.201 | 1.00 | 39.69 | C |
| ATOM | 5080 | CD | PRO | R | 83 | 31.911 | 83.675 | 176.259 | 1.00 | 38.22 | C |
| ATOM | 5083 | C | PRO | R | 83 | 29.312 | 82.749 | 178.299 | 1.00 | 32.61 | C |
| ATOM | 5084 | O | PRO | R | 83 | 29.492 | 83.883 | 178.745 | 1.00 | 32.18 | O |
| ATOM | 5085 | N | CYS | R | 84 | 28.366 | 81.942 | 178.761 | 1.00 | 31.26 | N |
| ATOM | 5086 | CA | CYS | R | 84 | 27.512 | 82.343 | 179.876 | 1.00 | 32.99 | C |
| ATOM | 5088 | CB | CYS | R | 84 | 26.368 | 81.357 | 180.045 | 1.00 | 36.52 | C |
| ATOM | 5091 | SG | CYS | R | 84 | 25.289 | 81.326 | 178.581 | 1.00 | 43.19 | S |
| ATOM | 5093 | C | CYS | R | 84 | 28.305 | 82.432 | 181.177 | 1.00 | 32.63 | C |
| ATOM | 5094 | O | CYS | R | 84 | 29.269 | 81.699 | 181.377 | 1.00 | 29.51 | O |
| ATOM | 5096 | N | THR | R | 85 | 27.871 | 83.328 | 182.056 | 1.00 | 30.21 | N |
| ATOM | 5097 | CA | THR | R | 85 | 28.542 | 83.589 | 183.326 | 1.00 | 30.58 | C |
| ATOM | 5099 | CB | THR | R | 85 | 28.244 | 85.034 | 183.784 | 1.00 | 32.58 | C |
| ATOM | 5101 | OG1 | THR | R | 85 | 28.590 | 85.947 | 182.727 | 1.00 | 33.09 | O |
| ATOM | 5103 | CG2 | THR | R | 85 | 29.045 | 85.390 | 185.029 | 1.00 | 34.68 | C |
| ATOM | 5107 | C | THR | R | 85 | 28.098 | 82.633 | 184.435 | 1.00 | 29.84 | C |
| ATOM | 5108 | O | THR | R | 85 | 26.916 | 82.282 | 184.536 | 1.00 | 31.33 | O |
| ATOM | 5110 | N | TRP | R | 86 | 29.048 | 82.234 | 185.273 | 1.00 | 31.33 | N |
| ATOM | 5111 | CA | TRP | R | 86 | 28.755 | 81.560 | 186.539 | 1.00 | 30.53 | C |
| ATOM | 5113 | CB | TRP | R | 86 | 29.928 | 80.687 | 186.974 | 1.00 | 35.45 | C |
| ATOM | 5116 | CG | TRP | R | 86 | 30.353 | 79.627 | 186.023 | 1.00 | 35.47 | C |
| ATOM | 5117 | CD1 | TRP | R | 86 | 31.394 | 79.682 | 185.155 | 1.00 | 36.03 | C |
| ATOM | 5119 | NE1 | TRP | R | 86 | 31.505 | 78.497 | 184.471 | 1.00 | 36.99 | N |
| ATOM | 5121 | CE2 | TRP | R | 86 | 30.526 | 77.644 | 184.901 | 1.00 | 35.80 | C |
| ATOM | 5122 | CD2 | TRP | R | 86 | 29.779 | 78.325 | 185.884 | 1.00 | 35.33 | C |
| ATOM | 5123 | CE3 | TRP | R | 86 | 28.704 | 77.670 | 186.491 | 1.00 | 38.29 | C |
| ATOM | 5125 | CZ3 | TRP | R | 86 | 28.408 | 76.365 | 186.092 | 1.00 | 38.82 | C |
| ATOM | 5127 | CH2 | TRP | R | 86 | 29.167 | 75.720 | 185.102 | 1.00 | 37.07 | C |
| ATOM | 5129 | CZ2 | TRP | R | 86 | 30.227 | 76.339 | 184.497 | 1.00 | 39.25 | C |
| ATOM | 5131 | C | TRP | R | 86 | 28.551 | 82.601 | 187.635 | 1.00 | 28.82 | C |
| ATOM | 5132 | O | TRP | R | 86 | 29.334 | 83.545 | 187.755 | 1.00 | 29.68 | O |
| ATOM | 5134 | N | CYS | R | 87 | 27.538 | 82.406 | 188.472 | 1.00 | 31.45 | N |
| ATOM | 5135 | CA | CYS | R | 87 | 27.371 | 83.250 | 189.653 | 1.00 | 30.36 | C |
| ATOM | 5137 | CB | CYS | R | 87 | 25.967 | 83.080 | 190.248 | 1.00 | 29.93 | C |
| ATOM | 5140 | SG | CYS | R | 87 | 24.587 | 83.214 | 189.068 | 1.00 | 33.16 | S |
| ATOM | 5142 | C | CYS | R | 87 | 28.442 | 82.892 | 190.698 | 1.00 | 29.84 | C |
| ATOM | 5143 | O | CYS | R | 87 | 28.758 | 81.714 | 190.894 | 1.00 | 30.21 | O |
| ATOM | 5145 | N | ASN | R | 88 | 28.999 | 83.902 | 191.363 | 1.00 | 32.19 | N |
| ATOM | 5146 | CA | ASN | R | 88 | 29.993 | 83.680 | 192.423 | 1.00 | 31.85 | C |
| ATOM | 5148 | CB | ASN | R | 88 | 30.908 | 84.900 | 192.582 | 1.00 | 33.21 | C |
| ATOM | 5151 | CG | ASN | R | 88 | 32.073 | 84.651 | 193.539 | 1.00 | 33.14 | C |
| ATOM | 5152 | OD1 | ASN | R | 88 | 32.122 | 83.640 | 194.249 | 1.00 | 37.17 | O |
| ATOM | 5153 | ND2 | ASN | R | 88 | 33.021 | 85.575 | 193.553 | 1.00 | 35.10 | N |
| ATOM | 5156 | C | ASN | R | 88 | 29.308 | 83.333 | 193.748 | 1.00 | 33.56 | C |
| ATOM | 5157 | O | ASN | R | 88 | 29.074 | 84.205 | 194.596 | 1.00 | 33.85 | O |
| ATOM | 5159 | N | LEU | R | 89 | 29.010 | 82.046 | 193.909 | 1.00 | 32.16 | N |
| ATOM | 5160 | CA | LEU | R | 89 | 28.316 | 81.514 | 195.077 | 1.00 | 34.67 | C |
| ATOM | 5162 | CB | LEU | R | 89 | 28.239 | 79.991 | 194.976 | 1.00 | 34.99 | C |
| ATOM | 5165 | CG | LEU | R | 89 | 27.400 | 79.261 | 196.026 | 1.00 | 33.60 | C |
| ATOM | 5167 | CD1 | LEU | R | 89 | 25.897 | 79.385 | 195.721 | 1.00 | 35.01 | C |
| ATOM | 5171 | CD2 | LEU | R | 89 | 27.812 | 77.794 | 196.095 | 1.00 | 34.74 | C |
| ATOM | 5175 | C | LEU | R | 89 | 28.970 | 81.897 | 196.406 | 1.00 | 36.45 | C |
| ATOM | 5176 | O | LEU | R | 89 | 28.287 | 82.344 | 197.326 | 1.00 | 36.03 | O |
| ATOM | 5178 | N | ARG | R | 90 | 30.286 | 81.719 | 196.498 | 1.00 | 39.08 | N |
| ATOM | 5179 | CA | ARG | R | 90 | 31.030 | 81.998 | 197.736 | 1.00 | 41.48 | C |
| ATOM | 5181 | CB | ARG | R | 90 | 32.503 | 81.600 | 197.584 | 1.00 | 47.97 | C |
| ATOM | 5184 | CG | ARG | R | 90 | 32.752 | 80.097 | 197.415 | 1.00 | 50.87 | C |
| ATOM | 5187 | CD | ARG | R | 90 | 34.176 | 79.843 | 196.895 | 1.00 | 52.39 | C |
| ATOM | 5190 | NE | ARG | R | 90 | 34.496 | 78.420 | 196.734 | 1.00 | 54.25 | N |
| ATOM | 5192 | CZ | ARG | R | 90 | 34.223 | 77.680 | 195.656 | 1.00 | 56.23 | C |
| ATOM | 5193 | NH1 | ARG | R | 90 | 33.599 | 78.192 | 194.598 | 1.00 | 55.29 | N |
| ATOM | 5196 | NH2 | ARG | R | 90 | 34.573 | 76.397 | 195.641 | 1.00 | 57.44 | N |
| ATOM | 5199 | C | ARG | R | 90 | 30.941 | 83.461 | 198.187 | 1.00 | 39.02 | C |
| ATOM | 5200 | O | ARG | R | 90 | 31.070 | 83.748 | 199.378 | 1.00 | 36.94 | O |
| ATOM | 5202 | N | SER | R | 91 | 30.729 | 84.381 | 197.246 | 1.00 | 36.53 | N |
| ATOM | 5203 | CA | SER | R | 91 | 30.575 | 85.801 | 197.582 | 1.00 | 34.97 | C |
| ATOM | 5205 | CB | SER | R | 91 | 30.965 | 86.680 | 196.388 | 1.00 | 34.58 | C |
| ATOM | 5208 | OG | SER | R | 91 | 29.942 | 86.723 | 195.406 | 1.00 | 32.03 | O |
| ATOM | 5210 | C | SER | R | 91 | 29.160 | 86.165 | 198.058 | 1.00 | 33.52 | C |
| ATOM | 5211 | O | SER | R | 91 | 28.936 | 87.275 | 198.540 | 1.00 | 34.22 | O |
| ATOM | 5213 | N | GLY | R | 92 | 28.214 | 85.239 | 197.922 | 1.00 | 34.38 | N |
| ATOM | 5214 | CA | GLY | R | 92 | 26.822 | 85.482 | 198.299 | 1.00 | 33.58 | C |
| ATOM | 5217 | C | GLY | R | 92 | 25.847 | 85.553 | 197.135 | 1.00 | 31.97 | C |
| ATOM | 5218 | O | GLY | R | 92 | 24.671 | 85.864 | 197.327 | 1.00 | 28.82 | O |
| ATOM | 5220 | N | SER | R | 93 | 26.329 | 85.273 | 195.929 | 1.00 | 30.57 | N |
| ATOM | 5221 | CA | SER | R | 93 | 25.482 | 85.267 | 194.747 | 1.00 | 31.84 | C |
| ATOM | 5223 | CB | SER | R | 93 | 26.312 | 85.482 | 193.478 | 1.00 | 32.12 | C |
| ATOM | 5226 | OG | SER | R | 93 | 25.476 | 85.572 | 192.346 | 1.00 | 30.24 | O |
| ATOM | 5228 | C | SER | R | 93 | 24.719 | 83.949 | 194.645 | 1.00 | 30.80 | C |
| ATOM | 5229 | O | SER | R | 93 | 25.158 | 82.921 | 195.158 | 1.00 | 32.24 | O |
| ATOM | 5231 | N | GLU | R | 94 | 23.562 | 84.001 | 193.999 | 1.00 | 28.20 | N |
| ATOM | 5232 | CA | GLU | R | 94 | 22.786 | 82.804 | 193.709 | 1.00 | 28.68 | C |
| ATOM | 5234 | CB | GLU | R | 94 | 21.715 | 82.594 | 194.791 | 1.00 | 28.68 | C |
| ATOM | 5237 | CG | GLU | R | 94 | 20.923 | 81.280 | 194.672 | 1.00 | 30.10 | C |
| ATOM | 5240 | CD | GLU | R | 94 | 19.753 | 81.218 | 195.641 | 1.00 | 30.83 | C |
| ATOM | 5241 | OE1 | GLU | R | 94 | 19.957 | 80.879 | 196.827 | 1.00 | 30.39 | O |
| ATOM | 5242 | OE2 | GLU | R | 94 | 18.623 | 81.511 | 195.209 | 1.00 | 31.37 | O |
| ATOM | 5243 | C | GLU | R | 94 | 22.152 | 82.948 | 192.327 | 1.00 | 29.45 | C |
| ATOM | 5244 | O | GLU | R | 94 | 21.613 | 83.999 | 191.988 | 1.00 | 27.09 | O |
| ATOM | 5246 | N | ARG | R | 95 | 22.215 | 81.885 | 191.530 | 1.00 | 30.53 | N |
| ATOM | 5247 | CA | ARG | R | 95 | 21.600 | 81.898 | 190.212 | 1.00 | 31.60 | C |
| ATOM | 5249 | CB | ARG | R | 95 | 22.039 | 80.689 | 189.384 | 1.00 | 31.20 | C |
| ATOM | 5252 | CG | ARG | R | 95 | 21.537 | 80.718 | 187.951 | 1.00 | 33.93 | C |
| ATOM | 5255 | CD | ARG | R | 95 | 22.149 | 79.597 | 187.136 | 1.00 | 36.84 | C |
| ATOM | 5258 | NE | ARG | R | 95 | 21.421 | 78.348 | 187.290 | 1.00 | 41.45 | N |
| ATOM | 5260 | CZ | ARG | R | 95 | 21.904 | 77.141 | 186.994 | 1.00 | 42.48 | C |
| ATOM | 5261 | NH1 | ARG | R | 95 | 23.143 | 76.985 | 186.542 | 1.00 | 46.37 | N |
| ATOM | 5264 | NH2 | ARG | R | 95 | 21.138 | 76.079 | 187.173 | 1.00 | 43.76 | N |
| ATOM | 5267 | C | ARG | R | 95 | 20.093 | 81.904 | 190.341 | 1.00 | 30.92 | C |
| ATOM | 5268 | O | ARG | R | 95 | 19.521 | 81.055 | 191.022 | 1.00 | 29.45 | O |
| ATOM | 5270 | N | LYS | R | 96 | 19.460 | 82.874 | 189.689 | 1.00 | 29.39 | N |
| ATOM | 5271 | CA | LYS | R | 96 | 18.010 | 82.939 | 189.612 | 1.00 | 31.01 | C |
| ATOM | 5273 | CB | LYS | R | 96 | 17.545 | 84.395 | 189.523 | 1.00 | 31.86 | C |
| ATOM | 5276 | CG | LYS | R | 96 | 16.039 | 84.572 | 189.540 | 1.00 | 33.38 | C |
| ATOM | 5279 | CD | LYS | R | 96 | 15.659 | 86.037 | 189.609 | 1.00 | 33.79 | C |
| ATOM | 5282 | CE | LYS | R | 96 | 14.153 | 86.207 | 189.562 | 1.00 | 35.74 | C |
| ATOM | 5285 | NZ | LYS | R | 96 | 13.785 | 87.637 | 189.689 | 1.00 | 38.04 | N |
| ATOM | 5289 | C | LYS | R | 96 | 17.529 | 82.162 | 188.397 | 1.00 | 30.86 | C |
| ATOM | 5290 | O | LYS | R | 96 | 16.619 | 81.335 | 188.509 | 1.00 | 32.68 | O |
| ATOM | 5292 | N | GLN | R | 97 | 18.132 | 82.443 | 187.241 | 1.00 | 31.11 | N |
| ATOM | 5293 | CA | GLN | R | 97 | 17.725 | 81.859 | 185.965 | 1.00 | 35.00 | C |
| ATOM | 5295 | CB | GLN | R | 97 | 16.891 | 82.861 | 185.152 | 1.00 | 39.06 | C |
| ATOM | 5298 | CG | GLN | R | 97 | 15.790 | 83.559 | 185.932 | 1.00 | 44.63 | C |
| ATOM | 5301 | CD | GLN | R | 97 | 15.034 | 84.575 | 185.094 | 1.00 | 47.48 | C |
| ATOM | 5302 | OE1 | GLN | R | 97 | 14.531 | 84.255 | 184.018 | 1.00 | 52.28 | O |
| ATOM | 5303 | NE2 | GLN | R | 97 | 14.949 | 85.810 | 185.589 | 1.00 | 51.58 | N |
| ATOM | 5306 | C | GLN | R | 97 | 18.929 | 81.459 | 185.123 | 1.00 | 34.03 | C |
| ATOM | 5307 | O | GLN | R | 97 | 19.935 | 82.177 | 185.077 | 1.00 | 29.98 | O |
| ATOM | 5309 | N | LEU | R | 98 | 18.798 | 80.330 | 184.428 | 1.00 | 33.04 | N |
| ATOM | 5310 | CA | LEU | R | 98 | 19.792 | 79.884 | 183.454 | 1.00 | 34.97 | C |
| ATOM | 5312 | CB | LEU | R | 98 | 19.383 | 78.541 | 182.840 | 1.00 | 39.35 | C |
| ATOM | 5315 | CG | LEU | R | 98 | 19.609 | 77.284 | 183.679 | 1.00 | 44.11 | C |
| ATOM | 5317 | CD1 | LEU | R | 98 | 18.634 | 76.191 | 183.274 | 1.00 | 46.39 | C |
| ATOM | 5321 | CD2 | LEU | R | 98 | 21.049 | 76.808 | 183.540 | 1.00 | 45.60 | C |
| ATOM | 5325 | C | LEU | R | 98 | 19.942 | 80.895 | 182.323 | 1.00 | 31.37 | C |
| ATOM | 5326 | O | LEU | R | 98 | 18.984 | 81.574 | 181.951 | 1.00 | 27.93 | O |
| ATOM | 5328 | N | CYS | R | 99 | 21.144 | 80.982 | 181.767 | 1.00 | 30.66 | N |
| ATOM | 5329 | CA | CYS | R | 99 | 21.353 | 81.772 | 180.565 | 1.00 | 31.87 | C |
| ATOM | 5331 | CB | CYS | R | 99 | 22.820 | 81.780 | 180.174 | 1.00 | 42.55 | C |
| ATOM | 5334 | SG | CYS | R | 99 | 23.366 | 80.183 | 179.597 | 1.00 | 53.05 | S |
| ATOM | 5336 | C | CYS | R | 99 | 20.547 | 81.206 | 179.404 | 1.00 | 33.99 | C |
| ATOM | 5337 | O | CYS | R | 99 | 20.274 | 80.000 | 179.345 | 1.00 | 31.15 | O |
| ATOM | 5339 | N | THR | R | 100 | 20.145 | 82.094 | 178.502 | 1.00 | 33.12 | N |
| ATOM | 5340 | CA | THR | R | 100 | 19.486 | 81.708 | 177.264 | 1.00 | 34.14 | C |
| ATOM | 5342 | CB | THR | R | 100 | 18.064 | 82.308 | 177.153 | 1.00 | 35.64 | C |
| ATOM | 5344 | OG1 | THR | R | 100 | 18.154 | 83.714 | 176.907 | 1.00 | 33.56 | O |
| ATOM | 5346 | CG2 | THR | R | 100 | 17.259 | 82.061 | 178.425 | 1.00 | 39.29 | C |
| ATOM | 5350 | C | THR | R | 100 | 20.348 | 82.217 | 176.110 | 1.00 | 29.99 | C |
| ATOM | 5351 | O | THR | R | 100 | 21.344 | 82.917 | 176.330 | 1.00 | 30.86 | O |
| ATOM | 5353 | N | ALA | R | 101 | 19.948 | 81.896 | 174.884 | 1.00 | 32.24 | N |
| ATOM | 5354 | CA | ALA | R | 101 | 20.658 | 82.375 | 173.693 | 1.00 | 31.73 | C |
| ATOM | 5356 | CB | ALA | R | 101 | 20.008 | 81.824 | 172.427 | 1.00 | 34.95 | C |
| ATOM | 5360 | C | ALA | R | 101 | 20.716 | 83.899 | 173.637 | 1.00 | 30.60 | C |
| ATOM | 5361 | O | ALA | R | 101 | 21.653 | 84.466 | 173.074 | 1.00 | 28.59 | O |
| ATOM | 5363 | N | THR | R | 102 | 19.724 | 84.558 | 174.235 | 1.00 | 31.23 | N |
| ATOM | 5364 | CA | THR | R | 102 | 19.596 | 86.011 | 174.174 | 1.00 | 30.59 | C |
| ATOM | 5366 | CB | THR | R | 102 | 18.181 | 86.417 | 173.728 | 1.00 | 32.66 | C |
| ATOM | 5368 | OG1 | THR | R | 102 | 17.199 | 85.842 | 174.607 | 1.00 | 32.09 | O |
| ATOM | 5370 | CG2 | THR | R | 102 | 17.917 | 85.947 | 172.313 | 1.00 | 34.21 | C |
| ATOM | 5374 | C | THR | R | 102 | 19.924 | 86.727 | 175.487 | 1.00 | 31.72 | C |
| ATOM | 5375 | O | THR | R | 102 | 19.893 | 87.948 | 175.544 | 1.00 | 30.88 | O |
| ATOM | 5377 | N | GLN | R | 103 | 20.263 | 85.981 | 176.532 | 1.00 | 34.68 | N |
| ATOM | 5378 | CA | GLN | R | 103 | 20.408 | 86.572 | 177.855 | 1.00 | 34.55 | C |
| ATOM | 5380 | CB | GLN | R | 103 | 19.041 | 86.627 | 178.548 | 1.00 | 37.38 | C |
| ATOM | 5383 | CG | GLN | R | 103 | 18.986 | 87.556 | 179.744 | 1.00 | 42.14 | C |
| ATOM | 5386 | CD | GLN | R | 103 | 17.573 | 87.757 | 180.270 | 1.00 | 46.68 | C |
| ATOM | 5387 | OE1 | GLN | R | 103 | 16.780 | 86.809 | 180.357 | 1.00 | 50.40 | O |
| ATOM | 5388 | NE2 | GLN | R | 103 | 17.252 | 88.996 | 180.630 | 1.00 | 52.65 | N |
| ATOM | 5391 | C | GLN | R | 103 | 21.394 | 85.785 | 178.698 | 1.00 | 32.00 | C |
| ATOM | 5392 | O | GLN | R | 103 | 21.285 | 84.569 | 178.825 | 1.00 | 34.25 | O |
| ATOM | 5394 | N | ASP | R | 104 | 22.359 | 86.488 | 179.279 | 1.00 | 32.62 | N |
| ATOM | 5395 | CA | ASP | R | 104 | 23.301 | 85.868 | 180.197 | 1.00 | 31.86 | C |
| ATOM | 5397 | CB | ASP | R | 104 | 24.370 | 86.879 | 180.608 | 1.00 | 34.62 | C |
| ATOM | 5400 | CG | ASP | R | 104 | 25.624 | 86.230 | 181.141 | 1.00 | 31.61 | C |
| ATOM | 5401 | OD1 | ASP | R | 104 | 25.742 | 84.980 | 181.117 | 1.00 | 32.26 | O |
| ATOM | 5402 | OD2 | ASP | R | 104 | 26.501 | 86.987 | 181.591 | 1.00 | 34.77 | O |
| ATOM | 5403 | C | ASP | R | 104 | 22.561 | 85.344 | 181.436 | 1.00 | 30.54 | C |
| ATOM | 5404 | O | ASP | R | 104 | 21.425 | 85.727 | 181.705 | 1.00 | 28.69 | O |
| ATOM | 5406 | N | THR | R | 105 | 23.220 | 84.453 | 182.165 | 1.00 | 30.75 | N |
| ATOM | 5407 | CA | THR | R | 105 | 22.802 | 84.027 | 183.503 | 1.00 | 31.10 | C |
| ATOM | 5409 | CB | THR | R | 105 | 24.009 | 83.395 | 184.222 | 1.00 | 29.90 | C |
| ATOM | 5411 | OG1 | THR | R | 105 | 24.649 | 82.469 | 183.336 | 1.00 | 29.15 | O |
| ATOM | 5413 | CG2 | THR | R | 105 | 23.603 | 82.695 | 185.483 | 1.00 | 30.64 | C |
| ATOM | 5417 | C | THR | R | 105 | 22.296 | 85.187 | 184.361 | 1.00 | 29.39 | C |
| ATOM | 5418 | O | THR | R | 105 | 22.909 | 86.255 | 184.395 | 1.00 | 29.12 | O |
| ATOM | 5420 | N | VAL | R | 106 | 21.175 | 84.978 | 185.055 | 1.00 | 28.36 | N |
| ATOM | 5421 | CA | VAL | R | 106 | 20.680 | 85.958 | 186.009 | 1.00 | 30.23 | C |
| ATOM | 5423 | CB | VAL | R | 106 | 19.147 | 86.100 | 185.962 | 1.00 | 32.86 | C |
| ATOM | 5425 | CG1 | VAL | R | 106 | 18.680 | 87.183 | 186.937 | 1.00 | 34.77 | C |
| ATOM | 5429 | CG2 | VAL | R | 106 | 18.692 | 86.420 | 184.545 | 1.00 | 35.80 | C |
| ATOM | 5433 | C | VAL | R | 106 | 21.137 | 85.538 | 187.404 | 1.00 | 31.04 | C |
| ATOM | 5434 | O | VAL | R | 106 | 20.771 | 84.467 | 187.883 | 1.00 | 28.66 | O |
| ATOM | 5436 | N | CYS | R | 107 | 21.952 | 86.390 | 188.025 | 1.00 | 29.71 | N |
| ATOM | 5437 | CA | CYS | R | 107 | 22.520 | 86.161 | 189.352 | 1.00 | 31.37 | C |
| ATOM | 5439 | CB | CYS | R | 107 | 24.044 | 86.200 | 189.289 | 1.00 | 32.45 | C |
| ATOM | 5442 | SG | CYS | R | 107 | 24.809 | 85.034 | 188.154 | 1.00 | 33.53 | S |
| ATOM | 5444 | C | CYS | R | 107 | 22.066 | 87.275 | 190.278 | 1.00 | 32.20 | C |
| ATOM | 5445 | O | CYS | R | 107 | 22.016 | 88.433 | 189.869 | 1.00 | 31.22 | O |
| ATOM | 5447 | N | ARG | R | 108 | 21.757 | 86.933 | 191.524 | 1.00 | 31.12 | N |
| ATOM | 5448 | CA | ARG | R | 108 | 21.321 | 87.932 | 192.508 | 1.00 | 31.59 | C |
| ATOM | 5450 | CB | ARG | R | 108 | 19.819 | 87.823 | 192.742 | 1.00 | 29.80 | C |
| ATOM | 5453 | CG | ARG | R | 108 | 18.969 | 88.126 | 191.518 | 1.00 | 31.23 | C |
| ATOM | 5456 | CD | ARG | R | 108 | 18.974 | 89.596 | 191.172 | 1.00 | 32.30 | C |
| ATOM | 5459 | NE | ARG | R | 108 | 18.115 | 89.884 | 190.028 | 1.00 | 31.10 | N |
| ATOM | 5461 | CZ | ARG | R | 108 | 18.530 | 90.129 | 188.782 | 1.00 | 36.67 | C |
| ATOM | 5462 | NH1 | ARG | R | 108 | 19.827 | 90.146 | 188.459 | 1.00 | 36.87 | N |
| ATOM | 5465 | NH2 | ARG | R | 108 | 17.625 | 90.374 | 187.839 | 1.00 | 39.52 | N |
| ATOM | 5468 | C | ARG | R | 108 | 22.063 | 87.791 | 193.828 | 1.00 | 29.67 | C |
| ATOM | 5469 | O | ARG | R | 108 | 22.314 | 86.685 | 194.297 | 1.00 | 29.04 | O |
| ATOM | 5471 | N | CYS | R | 109 | 22.402 | 88.925 | 194.430 | 1.00 | 30.97 | N |
| ATOM | 5472 | CA | CYS | R | 109 | 23.082 | 88.936 | 195.716 | 1.00 | 33.71 | C |
| ATOM | 5474 | CB | CYS | R | 109 | 23.827 | 90.261 | 195.915 | 1.00 | 33.71 | C |
| ATOM | 5477 | SG | CYS | R | 109 | 25.179 | 90.492 | 194.745 | 1.00 | 36.63 | S |
| ATOM | 5479 | C | CYS | R | 109 | 22.079 | 88.685 | 196.848 | 1.00 | 34.61 | C |
| ATOM | 5480 | O | CYS | R | 109 | 20.978 | 89.241 | 196.852 | 1.00 | 36.09 | O |
| ATOM | 5482 | N | ARG | R | 110 | 22.465 | 87.818 | 197.783 | 1.00 | 34.30 | N |
| ATOM | 5483 | CA | ARG | R | 110 | 21.628 | 87.475 | 198.940 | 1.00 | 36.90 | C |
| ATOM | 5485 | CB | ARG | R | 110 | 22.131 | 86.187 | 199.616 | 1.00 | 38.35 | C |
| ATOM | 5488 | CG | ARG | R | 110 | 23.456 | 86.323 | 200.363 | 1.00 | 38.87 | C |
| ATOM | 5491 | CD | ARG | R | 110 | 23.868 | 85.038 | 201.086 | 1.00 | 42.34 | C |
| ATOM | 5494 | NE | ARG | R | 110 | 24.918 | 85.330 | 202.067 | 1.00 | 44.29 | N |
| ATOM | 5496 | CZ | ARG | R | 110 | 26.151 | 84.813 | 202.098 | 1.00 | 47.50 | C |
| ATOM | 5497 | NH1 | ARG | R | 110 | 26.565 | 83.903 | 201.217 | 1.00 | 47.46 | N |
| ATOM | 5500 | NH2 | ARG | R | 110 | 26.987 | 85.205 | 203.054 | 1.00 | 49.10 | N |
| ATOM | 5503 | C | ARG | R | 110 | 21.600 | 88.613 | 199.952 | 1.00 | 35.72 | C |
| ATOM | 5504 | O | ARG | R | 110 | 22.377 | 89.578 | 199.851 | 1.00 | 33.85 | O |
| ATOM | 5506 | N | ALA | R | 111 | 20.709 | 88.492 | 200.935 | 1.00 | 34.36 | N |
| ATOM | 5507 | CA | ALA | R | 111 | 20.631 | 89.457 | 202.029 | 1.00 | 34.82 | C |
| ATOM | 5509 | CB | ALA | R | 111 | 19.518 | 89.069 | 203.018 | 1.00 | 34.10 | C |
| ATOM | 5513 | C | ALA | R | 111 | 21.969 | 89.563 | 202.755 | 1.00 | 33.97 | C |
| ATOM | 5514 | O | ALA | R | 111 | 22.663 | 88.565 | 202.948 | 1.00 | 32.06 | O |
| ATOM | 5516 | N | GLY | R | 112 | 22.321 | 90.783 | 203.153 | 1.00 | 33.85 | N |
| ATOM | 5517 | CA | GLY | R | 112 | 23.601 | 91.047 | 203.801 | 1.00 | 34.14 | C |
| ATOM | 5520 | C | GLY | R | 112 | 24.734 | 91.341 | 202.838 | 1.00 | 33.66 | C |
| ATOM | 5521 | O | GLY | R | 112 | 25.848 | 91.648 | 203.271 | 1.00 | 34.08 | O |
| ATOM | 5523 | N | THR | R | 113 | 24.463 | 91.241 | 201.536 | 1.00 | 34.60 | N |
| ATOM | 5524 | CA | THR | R | 113 | 25.455 | 91.534 | 200.507 | 1.00 | 34.09 | C |
| ATOM | 5526 | CB | THR | R | 113 | 26.033 | 90.241 | 199.870 | 1.00 | 35.49 | C |
| ATOM | 5528 | OG1 | THR | R | 113 | 25.050 | 89.613 | 199.038 | 1.00 | 36.76 | O |
| ATOM | 5530 | CG2 | THR | R | 113 | 26.479 | 89.256 | 200.948 | 1.00 | 33.94 | C |
| ATOM | 5534 | C | THR | R | 113 | 24.852 | 92.422 | 199.422 | 1.00 | 33.73 | C |
| ATOM | 5535 | O | THR | R | 113 | 23.630 | 92.542 | 199.315 | 1.00 | 33.16 | O |
| ATOM | 5537 | N | GLN | R | 114 | 25.722 | 93.059 | 198.638 | 1.00 | 34.70 | N |
| ATOM | 5538 | CA | GLN | R | 114 | 25.304 | 93.900 | 197.518 | 1.00 | 34.29 | C |
| ATOM | 5540 | CB | GLN | R | 114 | 25.336 | 95.379 | 197.914 | 1.00 | 33.57 | C |
| ATOM | 5543 | CG | GLN | R | 114 | 26.724 | 95.890 | 198.293 | 1.00 | 33.79 | C |
| ATOM | 5546 | CD | GLN | R | 114 | 26.770 | 97.389 | 198.512 | 1.00 | 36.08 | C |
| ATOM | 5547 | OE1 | GLN | R | 114 | 25.819 | 98.110 | 198.192 | 1.00 | 37.47 | O |
| ATOM | 5548 | NE2 | GLN | R | 114 | 27.882 | 97.868 | 199.067 | 1.00 | 34.57 | N |
| ATOM | 5551 | C | GLN | R | 114 | 26.217 | 93.672 | 196.310 | 1.00 | 34.87 | C |
| ATOM | 5552 | O | GLN | R | 114 | 27.356 | 93.225 | 196.474 | 1.00 | 35.45 | O |
| ATOM | 5554 | N | PRO | R | 115 | 25.726 | 93.986 | 195.096 | 1.00 | 34.42 | N |
| ATOM | 5555 | CA | PRO | R | 115 | 26.553 | 93.834 | 193.894 | 1.00 | 36.54 | C |
| ATOM | 5557 | CB | PRO | R | 115 | 25.613 | 94.271 | 192.760 | 1.00 | 36.34 | C |
| ATOM | 5560 | CG | PRO | R | 115 | 24.247 | 94.168 | 193.318 | 1.00 | 36.76 | C |
| ATOM | 5563 | CD | PRO | R | 115 | 24.380 | 94.487 | 194.768 | 1.00 | 34.77 | C |
| ATOM | 5566 | C | PRO | R | 115 | 27.808 | 94.712 | 193.902 | 1.00 | 36.97 | C |
| ATOM | 5567 | O | PRO | R | 115 | 27.755 | 95.868 | 194.333 | 1.00 | 36.24 | O |
| ATOM | 5568 | N | LEU | R | 116 | 28.921 | 94.155 | 193.431 | 1.00 | 39.67 | N |
| ATOM | 5569 | CA | LEU | R | 116 | 30.180 | 94.897 | 193.267 | 1.00 | 42.63 | C |
| ATOM | 5571 | CB | LEU | R | 116 | 31.372 | 93.928 | 193.214 | 1.00 | 43.90 | C |
| ATOM | 5574 | CG | LEU | R | 116 | 31.930 | 93.472 | 194.567 | 1.00 | 45.30 | C |
| ATOM | 5576 | CD1 | LEU | R | 116 | 32.553 | 92.086 | 194.480 | 1.00 | 46.21 | C |
| ATOM | 5580 | CD2 | LEU | R | 116 | 32.945 | 94.478 | 195.094 | 1.00 | 46.55 | C |
| ATOM | 5584 | C | LEU | R | 116 | 30.208 | 95.796 | 192.020 | 1.00 | 44.97 | C |
| ATOM | 5585 | O | LEU | R | 116 | 30.871 | 96.840 | 192.028 | 1.00 | 43.90 | O |
| ATOM | 5587 | N | ASP | R | 117 | 29.507 | 95.394 | 190.958 | 1.00 | 47.08 | N |
| ATOM | 5588 | CA | ASP | R | 117 | 29.596 | 96.082 | 189.658 | 1.00 | 46.73 | C |
| ATOM | 5590 | CB | ASP | R | 117 | 30.413 | 95.225 | 188.676 | 1.00 | 51.87 | C |
| ATOM | 5593 | CG | ASP | R | 117 | 31.034 | 96.038 | 187.543 | 1.00 | 53.86 | C |
| ATOM | 5594 | OD1 | ASP | R | 117 | 30.803 | 97.265 | 187.465 | 1.00 | 58.67 | O |
| ATOM | 5595 | OD2 | ASP | R | 117 | 31.767 | 95.445 | 186.719 | 1.00 | 59.45 | O |
| ATOM | 5596 | C | ASP | R | 117 | 28.217 | 96.375 | 189.061 | 1.00 | 46.50 | C |
| ATOM | 5597 | O | ASP | R | 117 | 27.221 | 95.742 | 189.428 | 1.00 | 46.99 | O |
| ATOM | 5599 | N | SER | R | 118 | 28.173 | 97.334 | 188.136 | 1.00 | 41.41 | N |
| ATOM | 5600 | CA | SER | R | 118 | 26.935 | 97.710 | 187.447 | 1.00 | 41.35 | C |
| ATOM | 5602 | CB | SER | R | 118 | 26.858 | 99.232 | 187.300 | 1.00 | 42.18 | C |
| ATOM | 5605 | OG | SER | R | 118 | 28.024 | 99.750 | 186.683 | 1.00 | 42.35 | O |
| ATOM | 5607 | C | SER | R | 118 | 26.768 | 97.050 | 186.070 | 1.00 | 40.03 | C |
| ATOM | 5608 | O | SER | R | 118 | 25.680 | 97.095 | 185.496 | 1.00 | 39.62 | O |
| ATOM | 5610 | N | TYR | R | 119 | 27.827 | 96.436 | 185.544 | 1.00 | 39.79 | N |
| ATOM | 5611 | CA | TYR | R | 119 | 27.783 | 95.851 | 184.192 | 1.00 | 38.77 | C |
| ATOM | 5613 | CB | TYR | R | 119 | 29.185 | 95.761 | 183.584 | 1.00 | 36.43 | C |
| ATOM | 5616 | CG | TYR | R | 119 | 29.701 | 97.088 | 183.091 | 1.00 | 35.89 | C |
| ATOM | 5617 | CD1 | TYR | R | 119 | 29.448 | 97.518 | 181.789 | 1.00 | 35.65 | C |
| ATOM | 5619 | CE1 | TYR | R | 119 | 29.920 | 98.744 | 181.333 | 1.00 | 35.02 | C |
| ATOM | 5621 | CZ | TYR | R | 119 | 30.651 | 99.553 | 182.187 | 1.00 | 35.42 | C |
| ATOM | 5622 | OH | TYR | R | 119 | 31.129 | 100.770 | 181.750 | 1.00 | 35.96 | O |
| ATOM | 5624 | CE2 | TYR | R | 119 | 30.910 | 99.145 | 183.485 | 1.00 | 35.55 | C |
| ATOM | 5626 | CD2 | TYR | R | 119 | 30.436 | 97.921 | 183.928 | 1.00 | 36.16 | C |
| ATOM | 5628 | C | TYR | R | 119 | 27.121 | 94.482 | 184.172 | 1.00 | 39.46 | C |
| ATOM | 5629 | O | TYR | R | 119 | 26.368 | 94.168 | 183.247 | 1.00 | 40.37 | O |
| ATOM | 5631 | N | LYS | R | 120 | 27.423 | 93.668 | 185.180 | 1.00 | 37.87 | N |
| ATOM | 5632 | CA | LYS | R | 120 | 26.752 | 92.386 | 185.380 | 1.00 | 38.93 | C |
| ATOM | 5634 | CB | LYS | R | 120 | 27.667 | 91.228 | 184.980 | 1.00 | 40.07 | C |
| ATOM | 5637 | CG | LYS | R | 120 | 27.982 | 91.187 | 183.491 | 1.00 | 40.92 | C |
| ATOM | 5640 | CD | LYS | R | 120 | 28.148 | 89.758 | 182.992 | 1.00 | 41.07 | C |
| ATOM | 5643 | CE | LYS | R | 120 | 28.199 | 89.694 | 181.475 | 1.00 | 40.99 | C |
| ATOM | 5646 | NZ | LYS | R | 120 | 26.841 | 89.625 | 180.866 | 1.00 | 41.79 | N |
| ATOM | 5650 | C | LYS | R | 120 | 26.350 | 92.275 | 186.844 | 1.00 | 37.40 | C |
| ATOM | 5651 | O | LYS | R | 120 | 26.941 | 91.496 | 187.594 | 1.00 | 39.96 | O |
| ATOM | 5653 | N | PRO | R | 121 | 25.343 | 93.065 | 187.260 | 1.00 | 37.59 | N |
| ATOM | 5654 | CA | PRO | R | 121 | 25.026 | 93.137 | 188.687 | 1.00 | 35.09 | C |
| ATOM | 5656 | CB | PRO | R | 121 | 23.927 | 94.209 | 188.778 | 1.00 | 36.47 | C |
| ATOM | 5659 | CG | PRO | R | 121 | 23.677 | 94.698 | 187.412 | 1.00 | 38.26 | C |
| ATOM | 5662 | CD | PRO | R | 121 | 24.449 | 93.893 | 186.431 | 1.00 | 37.76 | C |
| ATOM | 5665 | C | PRO | R | 121 | 24.531 | 91.811 | 189.240 | 1.00 | 33.21 | C |
| ATOM | 5666 | O | PRO | R | 121 | 23.612 | 91.209 | 188.680 | 1.00 | 35.26 | O |
| ATOM | 5667 | N | GLY | R | 122 | 25.156 | 91.352 | 190.320 | 1.00 | 33.34 | N |
| ATOM | 5668 | CA | GLY | R | 122 | 24.719 | 90.144 | 191.017 | 1.00 | 33.71 | C |
| ATOM | 5671 | C | GLY | R | 122 | 25.664 | 88.967 | 190.910 | 1.00 | 33.45 | C |
| ATOM | 5672 | O | GLY | R | 122 | 25.599 | 88.046 | 191.724 | 1.00 | 34.39 | O |
| ATOM | 5674 | N | VAL | R | 123 | 26.538 | 88.983 | 189.904 | 1.00 | 31.72 | N |
| ATOM | 5675 | CA | VAL | R | 123 | 27.501 | 87.900 | 189.708 | 1.00 | 32.92 | C |
| ATOM | 5677 | CB | VAL | R | 123 | 28.280 | 88.061 | 188.389 | 1.00 | 30.25 | C |
| ATOM | 5679 | CG1 | VAL | R | 123 | 29.405 | 87.031 | 188.280 | 1.00 | 31.28 | C |
| ATOM | 5683 | CG2 | VAL | R | 123 | 27.331 | 87.944 | 187.205 | 1.00 | 31.97 | C |
| ATOM | 5687 | C | VAL | R | 123 | 28.462 | 87.823 | 190.892 | 1.00 | 33.11 | C |
| ATOM | 5688 | O | VAL | R | 123 | 28.625 | 86.761 | 191.499 | 1.00 | 30.44 | O |
| ATOM | 5690 | N | ASP | R | 124 | 29.090 | 88.955 | 191.201 | 1.00 | 34.58 | N |
| ATOM | 5691 | CA | ASP | R | 124 | 29.994 | 89.089 | 192.336 | 1.00 | 36.79 | C |
| ATOM | 5693 | CB | ASP | R | 124 | 31.327 | 89.723 | 191.920 | 1.00 | 38.94 | C |
| ATOM | 5696 | CG | ASP | R | 124 | 32.078 | 88.915 | 190.876 | 1.00 | 43.88 | C |
| ATOM | 5697 | OD1 | ASP | R | 124 | 32.304 | 87.705 | 191.081 | 1.00 | 45.60 | O |
| ATOM | 5698 | OD2 | ASP | R | 124 | 32.475 | 89.513 | 189.855 | 1.00 | 47.12 | O |
| ATOM | 5699 | C | ASP | R | 124 | 29.331 | 90.001 | 193.358 | 1.00 | 34.55 | C |
| ATOM | 5700 | O | ASP | R | 124 | 28.842 | 91.082 | 193.011 | 1.00 | 32.54 | O |
| ATOM | 5702 | N | CYS | R | 125 | 29.331 | 89.565 | 194.613 | 1.00 | 35.07 | N |
| ATOM | 5703 | CA | CYS | R | 125 | 28.716 | 90.319 | 195.699 | 1.00 | 34.81 | C |
| ATOM | 5705 | CB | CYS | R | 125 | 27.569 | 89.507 | 196.297 | 1.00 | 33.47 | C |
| ATOM | 5708 | SG | CYS | R | 125 | 26.399 | 88.909 | 195.059 | 1.00 | 33.91 | S |
| ATOM | 5710 | C | CYS | R | 125 | 29.735 | 90.661 | 196.783 | 1.00 | 33.70 | C |
| ATOM | 5711 | O | CYS | R | 125 | 30.794 | 90.046 | 196.862 | 1.00 | 32.65 | O |
| ATOM | 5713 | N | ALA | R | 126 | 29.403 | 91.649 | 197.610 | 1.00 | 35.35 | N |
| ATOM | 5714 | CA | ALA | R | 126 | 30.257 | 92.051 | 198.732 | 1.00 | 36.56 | C |
| ATOM | 5716 | CB | ALA | R | 126 | 31.052 | 93.289 | 198.371 | 1.00 | 37.14 | C |
| ATOM | 5720 | C | ALA | R | 126 | 29.403 | 92.313 | 199.973 | 1.00 | 37.30 | C |
| ATOM | 5721 | O | ALA | R | 126 | 28.288 | 92.823 | 199.848 | 1.00 | 37.10 | O |
| ATOM | 5723 | N | PRO | R | 127 | 29.921 | 91.975 | 201.174 | 1.00 | 37.98 | N |
| ATOM | 5724 | CA | PRO | R | 127 | 29.147 | 92.219 | 202.392 | 1.00 | 38.60 | C |
| ATOM | 5726 | CB | PRO | R | 127 | 30.081 | 91.731 | 203.514 | 1.00 | 39.67 | C |
| ATOM | 5729 | CG | PRO | R | 127 | 31.055 | 90.831 | 202.858 | 1.00 | 40.45 | C |
| ATOM | 5732 | CD | PRO | R | 127 | 31.233 | 91.370 | 201.474 | 1.00 | 39.31 | C |
| ATOM | 5735 | C | PRO | R | 127 | 28.836 | 93.701 | 202.587 | 1.00 | 37.68 | C |
| ATOM | 5736 | O | PRO | R | 127 | 29.652 | 94.553 | 202.216 | 1.00 | 37.62 | O |
| ATOM | 5737 | N | CYS | R | 128 | 27.671 | 94.007 | 203.155 | 1.00 | 35.33 | N |
| ATOM | 5738 | CA | CYS | R | 128 | 27.347 | 95.391 | 203.504 | 1.00 | 38.24 | C |
| ATOM | 5740 | CB | CYS | R | 128 | 25.974 | 95.502 | 204.183 | 1.00 | 39.95 | C |
| ATOM | 5743 | SG | CYS | R | 128 | 24.532 | 95.413 | 203.077 | 1.00 | 42.04 | S |
| ATOM | 5745 | C | CYS | R | 128 | 28.429 | 95.927 | 204.438 | 1.00 | 36.38 | C |
| ATOM | 5746 | O | CYS | R | 128 | 28.931 | 95.185 | 205.285 | 1.00 | 36.04 | O |
| ATOM | 5748 | N | PRO | R | 129 | 28.815 | 97.206 | 204.271 | 1.00 | 37.38 | N |
| ATOM | 5749 | CA | PRO | R | 129 | 29.808 | 97.812 | 205.158 | 1.00 | 38.38 | C |
| ATOM | 5751 | CB | PRO | R | 129 | 30.158 | 99.131 | 204.455 | 1.00 | 38.85 | C |
| ATOM | 5754 | CG | PRO | R | 129 | 28.993 | 99.443 | 203.600 | 1.00 | 38.50 | C |
| ATOM | 5757 | CD | PRO | R | 129 | 28.339 | 98.146 | 203.238 | 1.00 | 38.66 | C |
| ATOM | 5760 | C | PRO | R | 129 | 29.218 | 98.061 | 206.548 | 1.00 | 39.28 | C |
| ATOM | 5761 | O | PRO | R | 129 | 28.021 | 97.828 | 206.742 | 1.00 | 38.85 | O |
| ATOM | 5762 | N | PRO | R | 130 | 30.048 | 98.512 | 207.513 | 1.00 | 39.28 | N |
| ATOM | 5763 | CA | PRO | R | 130 | 29.552 | 98.802 | 208.861 | 1.00 | 39.71 | C |
| ATOM | 5765 | CB | PRO | R | 130 | 30.751 | 99.478 | 209.544 | 1.00 | 38.66 | C |
| ATOM | 5768 | CG | PRO | R | 130 | 31.937 | 98.982 | 208.814 | 1.00 | 39.18 | C |
| ATOM | 5771 | CD | PRO | R | 130 | 31.499 | 98.762 | 207.397 | 1.00 | 39.87 | C |
| ATOM | 5774 | C | PRO | R | 130 | 28.343 | 99.742 | 208.863 | 1.00 | 40.99 | C |
| ATOM | 5775 | O | PRO | R | 130 | 28.358 | 100.765 | 208.176 | 1.00 | 44.25 | O |
| ATOM | 5776 | N | GLY | R | 131 | 27.307 | 99.383 | 209.619 | 1.00 | 38.86 | N |
| ATOM | 5777 | CA | GLY | R | 131 | 26.138 | 100.245 | 209.788 | 1.00 | 36.80 | C |
| ATOM | 5780 | C | GLY | R | 131 | 25.198 | 100.310 | 208.603 | 1.00 | 35.88 | C |
| ATOM | 5781 | O | GLY | R | 131 | 24.395 | 101.241 | 208.498 | 1.00 | 35.38 | O |
| ATOM | 5783 | N | HIS | R | 132 | 25.285 | 99.321 | 207.721 | 1.00 | 35.26 | N |
| ATOM | 5784 | CA | HIS | R | 132 | 24.432 | 99.260 | 206.538 | 1.00 | 36.04 | C |
| ATOM | 5786 | CB | HIS | R | 132 | 25.272 | 99.481 | 205.274 | 1.00 | 36.55 | C |
| ATOM | 5789 | CG | HIS | R | 132 | 25.841 | 100.862 | 205.169 | 1.00 | 38.37 | C |
| ATOM | 5790 | ND1 | HIS | R | 132 | 26.854 | 101.315 | 205.988 | 1.00 | 39.83 | N |
| ATOM | 5792 | CE1 | HIS | R | 132 | 27.143 | 102.566 | 205.677 | 1.00 | 40.38 | C |
| ATOM | 5794 | NE2 | HIS | R | 132 | 26.352 | 102.943 | 204.689 | 1.00 | 40.89 | N |
| ATOM | 5796 | CD2 | HIS | R | 132 | 25.526 | 101.897 | 204.354 | 1.00 | 39.73 | C |
| ATOM | 5798 | C | HIS | R | 132 | 23.685 | 97.932 | 206.476 | 1.00 | 34.75 | C |
| ATOM | 5799 | O | HIS | R | 132 | 24.119 | 96.939 | 207.076 | 1.00 | 34.24 | O |
| ATOM | 5801 | N | PHE | R | 133 | 22.546 | 97.941 | 205.775 | 1.00 | 35.10 | N |
| ATOM | 5802 | CA | PHE | R | 133 | 21.748 | 96.734 | 205.538 | 1.00 | 35.58 | C |
| ATOM | 5804 | CB | PHE | R | 133 | 20.478 | 96.745 | 206.403 | 1.00 | 35.67 | C |
| ATOM | 5807 | CG | PHE | R | 133 | 19.386 | 97.640 | 205.874 | 1.00 | 35.68 | C |
| ATOM | 5808 | CD1 | PHE | R | 133 | 19.420 | 99.012 | 206.105 | 1.00 | 36.17 | C |
| ATOM | 5810 | CE1 | PHE | R | 133 | 18.416 | 99.843 | 205.618 | 1.00 | 35.65 | C |
| ATOM | 5812 | CZ | PHE | R | 133 | 17.366 | 99.303 | 204.889 | 1.00 | 36.03 | C |
| ATOM | 5814 | CE2 | PHE | R | 133 | 17.320 | 97.937 | 204.654 | 1.00 | 35.75 | C |
| ATOM | 5816 | CD2 | PHE | R | 133 | 18.328 | 97.113 | 205.144 | 1.00 | 35.77 | C |
| ATOM | 5818 | C | PHE | R | 133 | 21.345 | 96.601 | 204.071 | 1.00 | 34.57 | C |
| ATOM | 5819 | O | PHE | R | 133 | 21.178 | 97.599 | 203.364 | 1.00 | 31.08 | O |
| ATOM | 5821 | N | SER | R | 134 | 21.174 | 95.356 | 203.631 | 1.00 | 34.02 | N |
| ATOM | 5822 | CA | SER | R | 134 | 20.526 | 95.066 | 202.360 | 1.00 | 35.43 | C |
| ATOM | 5824 | CB | SER | R | 134 | 21.561 | 94.850 | 201.255 | 1.00 | 35.12 | C |
| ATOM | 5827 | OG | SER | R | 134 | 20.925 | 94.484 | 200.040 | 1.00 | 36.26 | O |
| ATOM | 5829 | C | SER | R | 134 | 19.653 | 93.820 | 202.503 | 1.00 | 34.91 | C |
| ATOM | 5830 | O | SER | R | 134 | 20.082 | 92.840 | 203.110 | 1.00 | 33.90 | O |
| ATOM | 5832 | N | PRO | R | 135 | 18.430 | 93.847 | 201.937 | 1.00 | 36.45 | N |
| ATOM | 5833 | CA | PRO | R | 135 | 17.590 | 92.645 | 201.943 | 1.00 | 37.13 | C |
| ATOM | 5835 | CB | PRO | R | 135 | 16.175 | 93.187 | 201.684 | 1.00 | 39.77 | C |
| ATOM | 5838 | CG | PRO | R | 135 | 16.333 | 94.619 | 201.287 | 1.00 | 40.31 | C |
| ATOM | 5841 | CD | PRO | R | 135 | 17.778 | 94.974 | 201.247 | 1.00 | 37.77 | C |
| ATOM | 5844 | C | PRO | R | 135 | 17.968 | 91.627 | 200.863 | 1.00 | 37.11 | C |
| ATOM | 5845 | O | PRO | R | 135 | 17.358 | 90.560 | 200.786 | 1.00 | 36.05 | O |
| ATOM | 5846 | N | GLY | R | 136 | 18.962 | 91.953 | 200.040 | 1.00 | 37.34 | N |
| ATOM | 5847 | CA | GLY | R | 136 | 19.370 | 91.085 | 198.940 | 1.00 | 37.25 | C |
| ATOM | 5850 | C | GLY | R | 136 | 18.628 | 91.440 | 197.667 | 1.00 | 37.44 | C |
| ATOM | 5851 | O | GLY | R | 136 | 18.162 | 92.573 | 197.507 | 1.00 | 34.77 | O |
| ATOM | 5853 | N | ASP | R | 137 | 18.526 | 90.461 | 196.764 | 1.00 | 37.68 | N |
| ATOM | 5854 | CA | ASP | R | 137 | 17.913 | 90.642 | 195.440 | 1.00 | 36.12 | C |
| ATOM | 5856 | CB | ASP | R | 137 | 16.393 | 90.817 | 195.557 | 1.00 | 36.83 | C |
| ATOM | 5859 | CG | ASP | R | 137 | 15.668 | 90.564 | 194.236 | 1.00 | 39.67 | C |
| ATOM | 5860 | OD1 | ASP | R | 137 | 16.147 | 89.733 | 193.433 | 1.00 | 39.04 | O |
| ATOM | 5861 | OD2 | ASP | R | 137 | 14.616 | 91.197 | 194.000 | 1.00 | 41.55 | O |
| ATOM | 5862 | C | ASP | R | 137 | 18.532 | 91.814 | 194.664 | 1.00 | 33.91 | C |
| ATOM | 5863 | O | ASP | R | 137 | 17.844 | 92.508 | 193.913 | 1.00 | 33.24 | O |
| ATOM | 5865 | N | ASN | R | 138 | 19.837 | 92.004 | 194.849 | 1.00 | 34.54 | N |
| ATOM | 5866 | CA | ASN | R | 138 | 20.613 | 93.076 | 194.208 | 1.00 | 34.80 | C |
| ATOM | 5868 | CB | ASN | R | 138 | 20.511 | 93.007 | 192.667 | 1.00 | 33.64 | C |
| ATOM | 5871 | CG | ASN | R | 138 | 21.447 | 91.982 | 192.058 | 1.00 | 32.16 | C |
| ATOM | 5872 | OD1 | ASN | R | 138 | 22.085 | 91.199 | 192.761 | 1.00 | 31.59 | O |
| ATOM | 5873 | ND2 | ASN | R | 138 | 21.539 | 91.991 | 190.736 | 1.00 | 32.02 | N |
| ATOM | 5876 | C | ASN | R | 138 | 20.304 | 94.497 | 194.695 | 1.00 | 35.01 | C |
| ATOM | 5877 | O | ASN | R | 138 | 20.709 | 95.467 | 194.057 | 1.00 | 32.65 | O |
| ATOM | 5879 | N | GLN | R | 139 | 19.610 | 94.633 | 195.822 | 1.00 | 35.28 | N |
| ATOM | 5880 | CA | GLN | R | 139 | 19.386 | 95.958 | 196.397 | 1.00 | 36.66 | C |
| ATOM | 5882 | CB | GLN | R | 139 | 18.236 | 95.950 | 197.411 | 1.00 | 38.13 | C |
| ATOM | 5885 | CG | GLN | R | 139 | 16.866 | 95.917 | 196.748 | 1.00 | 39.52 | C |
| ATOM | 5888 | CD | GLN | R | 139 | 15.723 | 95.933 | 197.738 | 1.00 | 41.82 | C |
| ATOM | 5889 | OE1 | GLN | R | 139 | 15.647 | 96.804 | 198.610 | 1.00 | 41.21 | O |
| ATOM | 5890 | NE2 | GLN | R | 139 | 14.816 | 94.968 | 197.604 | 1.00 | 44.42 | N |
| ATOM | 5893 | C | GLN | R | 139 | 20.683 | 96.465 | 197.028 | 1.00 | 36.29 | C |
| ATOM | 5894 | O | GLN | R | 139 | 21.436 | 95.693 | 197.630 | 1.00 | 34.50 | O |
| ATOM | 5896 | N | ALA | R | 140 | 20.944 | 97.758 | 196.852 | 1.00 | 37.51 | N |
| ATOM | 5897 | CA | ALA | R | 140 | 22.126 | 98.412 | 197.415 | 1.00 | 37.94 | C |
| ATOM | 5899 | CB | ALA | R | 140 | 22.291 | 99.799 | 196.814 | 1.00 | 38.08 | C |
| ATOM | 5903 | C | ALA | R | 140 | 22.026 | 98.511 | 198.934 | 1.00 | 35.87 | C |
| ATOM | 5904 | O | ALA | R | 140 | 20.932 | 98.701 | 199.475 | 1.00 | 34.33 | O |
| ATOM | 5906 | N | CYS | R | 141 | 23.162 | 98.370 | 199.618 | 1.00 | 35.36 | N |
| ATOM | 5907 | CA | CYS | R | 141 | 23.205 | 98.551 | 201.071 | 1.00 | 37.17 | C |
| ATOM | 5909 | CB | CYS | R | 141 | 24.567 | 98.146 | 201.659 | 1.00 | 39.09 | C |
| ATOM | 5912 | SG | CYS | R | 141 | 25.095 | 96.413 | 201.382 | 1.00 | 41.32 | S |
| ATOM | 5914 | C | CYS | R | 141 | 22.910 | 100.022 | 201.391 | 1.00 | 37.26 | C |
| ATOM | 5915 | O | CYS | R | 141 | 23.400 | 100.918 | 200.699 | 1.00 | 35.92 | O |
| ATOM | 5917 | N | LYS | R | 142 | 22.096 | 100.252 | 202.424 | 1.00 | 37.15 | N |
| ATOM | 5918 | CA | LYS | R | 142 | 21.697 | 101.598 | 202.844 | 1.00 | 35.21 | C |
| ATOM | 5920 | CB | LYS | R | 142 | 20.197 | 101.800 | 202.625 | 1.00 | 36.09 | C |
| ATOM | 5923 | CG | LYS | R | 142 | 19.759 | 101.743 | 201.174 | 1.00 | 36.84 | C |
| ATOM | 5926 | CD | LYS | R | 142 | 18.258 | 101.959 | 201.039 | 1.00 | 36.53 | C |
| ATOM | 5929 | CE | LYS | R | 142 | 17.882 | 102.354 | 199.617 | 1.00 | 37.35 | C |
| ATOM | 5932 | NZ | LYS | R | 142 | 16.431 | 102.665 | 199.487 | 1.00 | 37.92 | N |
| ATOM | 5936 | C | LYS | R | 142 | 22.011 | 101.804 | 204.322 | 1.00 | 33.33 | C |
| ATOM | 5937 | O | LYS | R | 142 | 22.080 | 100.830 | 205.074 | 1.00 | 31.02 | O |
| ATOM | 5939 | N | PRO | R | 143 | 22.185 | 103.073 | 204.754 | 1.00 | 32.67 | N |
| ATOM | 5940 | CA | PRO | R | 143 | 22.446 | 103.338 | 206.172 | 1.00 | 33.72 | C |
| ATOM | 5942 | CB | PRO | R | 143 | 22.622 | 104.866 | 206.232 | 1.00 | 32.29 | C |
| ATOM | 5945 | CG | PRO | R | 143 | 22.805 | 105.318 | 204.840 | 1.00 | 32.99 | C |
| ATOM | 5948 | CD | PRO | R | 143 | 22.143 | 104.312 | 203.957 | 1.00 | 32.79 | C |
| ATOM | 5951 | C | PRO | R | 143 | 21.280 | 102.923 | 207.065 | 1.00 | 33.07 | C |
| ATOM | 5952 | O | PRO | R | 143 | 20.124 | 103.103 | 206.683 | 1.00 | 33.29 | O |
| ATOM | 5953 | N | TRP | R | 144 | 21.583 | 102.382 | 208.244 | 1.00 | 35.07 | N |
| ATOM | 5954 | CA | TRP | R | 144 | 20.545 | 102.094 | 209.241 | 1.00 | 36.35 | C |
| ATOM | 5956 | CB | TRP | R | 144 | 21.131 | 101.454 | 210.506 | 1.00 | 39.11 | C |
| ATOM | 5959 | CG | TRP | R | 144 | 21.780 | 100.122 | 210.313 | 1.00 | 39.33 | C |
| ATOM | 5960 | CD1 | TRP | R | 144 | 21.534 | 99.220 | 209.322 | 1.00 | 39.53 | C |
| ATOM | 5962 | NE1 | TRP | R | 144 | 22.318 | 98.106 | 209.490 | 1.00 | 39.66 | N |
| ATOM | 5964 | CE2 | TRP | R | 144 | 23.085 | 98.267 | 210.614 | 1.00 | 39.70 | C |
| ATOM | 5965 | CD2 | TRP | R | 144 | 22.766 | 99.526 | 211.164 | 1.00 | 39.80 | C |
| ATOM | 5966 | CE3 | TRP | R | 144 | 23.417 | 99.939 | 212.335 | 1.00 | 40.29 | C |
| ATOM | 5968 | CZ3 | TRP | R | 144 | 24.354 | 99.090 | 212.912 | 1.00 | 39.93 | C |
| ATOM | 5970 | CH2 | TRP | R | 144 | 24.650 | 97.841 | 212.339 | 1.00 | 39.77 | C |
| ATOM | 5972 | CZ2 | TRP | R | 144 | 24.027 | 97.412 | 211.194 | 1.00 | 39.66 | C |
| ATOM | 5974 | C | TRP | R | 144 | 19.836 | 103.379 | 209.655 | 1.00 | 35.60 | C |
| ATOM | 5975 | O | TRP | R | 144 | 20.467 | 104.432 | 209.776 | 1.00 | 35.43 | O |
| ATOM | 5977 | N | THR | R | 145 | 18.527 | 103.284 | 209.873 | 1.00 | 35.80 | N |
| ATOM | 5978 | CA | THR | R | 145 | 17.755 | 104.383 | 210.445 | 1.00 | 36.17 | C |
| ATOM | 5980 | CB | THR | R | 145 | 16.232 | 104.119 | 210.360 | 1.00 | 35.41 | C |
| ATOM | 5982 | OG1 | THR | R | 145 | 15.851 | 103.943 | 208.990 | 1.00 | 34.30 | O |
| ATOM | 5984 | CG2 | THR | R | 145 | 15.439 | 105.280 | 210.954 | 1.00 | 35.74 | C |
| ATOM | 5988 | C | THR | R | 145 | 18.159 | 104.571 | 211.908 | 1.00 | 36.85 | C |
| ATOM | 5989 | O | THR | R | 145 | 18.229 | 103.603 | 212.672 | 1.00 | 36.30 | O |
| ATOM | 5991 | N | ASN | R | 146 | 18.430 | 105.820 | 212.282 | 1.00 | 37.69 | N |
| ATOM | 5992 | CA | ASN | R | 146 | 18.862 | 106.162 | 213.637 | 1.00 | 37.04 | C |
| ATOM | 5994 | CB | ASN | R | 146 | 19.911 | 107.284 | 213.585 | 1.00 | 38.27 | C |
| ATOM | 5997 | CG | ASN | R | 146 | 20.493 | 107.617 | 214.954 | 1.00 | 38.84 | C |
| ATOM | 5998 | OD1 | ASN | R | 146 | 20.484 | 106.794 | 215.870 | 1.00 | 40.12 | O |
| ATOM | 5999 | ND2 | ASN | R | 146 | 21.010 | 108.832 | 215.092 | 1.00 | 39.29 | N |
| ATOM | 6002 | C | ASN | R | 146 | 17.669 | 106.576 | 214.501 | 1.00 | 36.00 | C |
| ATOM | 6003 | O | ASN | R | 146 | 17.271 | 107.744 | 214.511 | 1.00 | 34.48 | O |
| ATOM | 6005 | N | CYS | R | 147 | 17.103 | 105.603 | 215.216 | 1.00 | 34.89 | N |
| ATOM | 6006 | CA | CYS | R | 147 | 15.955 | 105.838 | 216.098 | 1.00 | 36.41 | C |
| ATOM | 6008 | CB | CYS | R | 147 | 15.513 | 104.530 | 216.759 | 1.00 | 34.05 | C |
| ATOM | 6011 | SG | CYS | R | 147 | 15.236 | 103.175 | 215.598 | 1.00 | 31.53 | S |
| ATOM | 6013 | C | CYS | R | 147 | 16.285 | 106.867 | 217.177 | 1.00 | 36.13 | C |
| ATOM | 6014 | O | CYS | R | 147 | 15.455 | 107.708 | 217.520 | 1.00 | 33.50 | O |
| ATOM | 6016 | N | THR | R | 148 | 17.510 | 106.788 | 217.691 | 1.00 | 38.64 | N |
| ATOM | 6017 | CA | THR | R | 148 | 18.005 | 107.720 | 218.695 | 1.00 | 40.44 | C |
| ATOM | 6019 | CB | THR | R | 148 | 19.060 | 107.051 | 219.600 | 1.00 | 40.73 | C |
| ATOM | 6021 | OG1 | THR | R | 148 | 20.239 | 106.750 | 218.844 | 1.00 | 41.31 | O |
| ATOM | 6023 | CG2 | THR | R | 148 | 18.503 | 105.759 | 220.187 | 1.00 | 40.81 | C |
| ATOM | 6027 | C | THR | R | 148 | 18.562 | 108.950 | 217.970 | 1.00 | 42.88 | C |
| ATOM | 6028 | O | THR | R | 148 | 19.737 | 109.013 | 217.601 | 1.00 | 43.31 | O |
| ATOM | 6030 | N | LEU | R | 149 | 17.685 | 109.928 | 217.779 | 1.00 | 43.91 | N |
| ATOM | 6031 | CA | LEU | R | 149 | 17.925 | 111.043 | 216.866 | 1.00 | 43.30 | C |
| ATOM | 6033 | CB | LEU | R | 149 | 18.763 | 110.526 | 215.571 | 0.00 | 30.00 | C |
| ATOM | 6036 | CG | LEU | R | 149 | 19.176 | 111.728 | 214.724 | 0.00 | 30.00 | C |
| ATOM | 6038 | CD1 | LEU | R | 149 | 19.236 | 112.990 | 215.582 | 0.00 | 30.00 | C |
| ATOM | 6042 | CD2 | LEU | R | 149 | 20.552 | 111.453 | 214.114 | 0.00 | 30.00 | C |
| ATOM | 6046 | C | LEU | R | 149 | 16.570 | 111.613 | 216.445 | 1.00 | 43.50 | C |
| ATOM | 6047 | O | LEU | R | 149 | 16.393 | 112.831 | 216.365 | 1.00 | 42.30 | O |
| ATOM | 6049 | N | ALA | R | 150 | 15.621 | 110.713 | 216.182 | 1.00 | 42.50 | N |
| ATOM | 6050 | CA | ALA | R | 150 | 14.216 | 111.076 | 215.995 | 1.00 | 41.83 | C |
| ATOM | 6052 | CB | ALA | R | 150 | 13.555 | 110.114 | 215.014 | 1.00 | 42.26 | C |
| ATOM | 6056 | C | ALA | R | 150 | 13.448 | 111.086 | 217.326 | 1.00 | 41.20 | C |
| ATOM | 6057 | O | ALA | R | 150 | 12.229 | 111.267 | 217.336 | 1.00 | 41.99 | O |
| ATOM | 6059 | N | GLY | R | 151 | 14.157 | 110.893 | 218.439 | 1.00 | 39.99 | N |
| ATOM | 6060 | CA | GLY | R | 151 | 13.550 | 110.884 | 219.769 | 1.00 | 39.46 | C |
| ATOM | 6063 | C | GLY | R | 151 | 12.732 | 109.636 | 220.055 | 1.00 | 39.07 | C |
| ATOM | 6064 | O | GLY | R | 151 | 11.733 | 109.699 | 220.775 | 1.00 | 38.53 | O |
| ATOM | 6066 | N | LYS | R | 152 | 13.167 | 108.503 | 219.502 | 1.00 | 38.38 | N |
| ATOM | 6067 | CA | LYS | R | 152 | 12.428 | 107.242 | 219.596 | 1.00 | 38.47 | C |
| ATOM | 6069 | CB | LYS | R | 152 | 11.860 | 106.856 | 218.224 | 1.00 | 38.26 | C |
| ATOM | 6072 | CG | LYS | R | 152 | 11.006 | 107.929 | 217.565 | 1.00 | 38.69 | C |
| ATOM | 6075 | CD | LYS | R | 152 | 10.329 | 107.401 | 216.309 | 1.00 | 38.61 | C |
| ATOM | 6078 | CE | LYS | R | 152 | 9.466 | 108.465 | 215.651 | 1.00 | 38.81 | C |
| ATOM | 6081 | NZ | LYS | R | 152 | 8.777 | 107.947 | 214.437 | 1.00 | 38.79 | N |
| ATOM | 6085 | C | LYS | R | 152 | 13.313 | 106.102 | 220.093 | 1.00 | 38.15 | C |
| ATOM | 6086 | O | LYS | R | 152 | 14.511 | 106.065 | 219.803 | 1.00 | 38.22 | O |
| ATOM | 6088 | N | HIS | R | 153 | 12.711 | 105.168 | 220.830 | 1.00 | 36.95 | N |
| ATOM | 6089 | CA | HIS | R | 153 | 13.381 | 103.919 | 221.195 | 1.00 | 35.80 | C |
| ATOM | 6091 | CB | HIS | R | 153 | 12.607 | 103.173 | 222.282 | 1.00 | 33.88 | C |
| ATOM | 6094 | CG | HIS | R | 153 | 12.609 | 103.861 | 223.609 | 1.00 | 33.93 | C |
| ATOM | 6095 | ND1 | HIS | R | 153 | 13.770 | 104.197 | 224.271 | 1.00 | 33.66 | N |
| ATOM | 6097 | CE1 | HIS | R | 153 | 13.466 | 104.784 | 225.416 | 1.00 | 32.96 | C |
| ATOM | 6099 | NE2 | HIS | R | 153 | 12.151 | 104.831 | 225.524 | 1.00 | 32.81 | N |
| ATOM | 6101 | CD2 | HIS | R | 153 | 11.591 | 104.258 | 224.408 | 1.00 | 32.74 | C |
| ATOM | 6103 | C | HIS | R | 153 | 13.507 | 103.008 | 219.978 | 1.00 | 34.67 | C |
| ATOM | 6104 | O | HIS | R | 153 | 12.787 | 103.175 | 218.989 | 1.00 | 34.28 | O |
| ATOM | 6106 | N | THR | R | 154 | 14.415 | 102.040 | 220.073 | 1.00 | 34.00 | N |
| ATOM | 6107 | CA | THR | R | 154 | 14.596 | 101.020 | 219.040 | 1.00 | 36.10 | C |
| ATOM | 6109 | CB | THR | R | 154 | 16.095 | 100.742 | 218.784 | 1.00 | 34.32 | C |
| ATOM | 6111 | OG1 | THR | R | 154 | 16.795 | 101.984 | 218.641 | 1.00 | 33.32 | O |
| ATOM | 6113 | CG2 | THR | R | 154 | 16.292 | 99.911 | 217.521 | 1.00 | 33.62 | C |
| ATOM | 6117 | C | THR | R | 154 | 13.899 | 99.726 | 219.476 | 1.00 | 37.51 | C |
| ATOM | 6118 | O | THR | R | 154 | 14.355 | 99.052 | 220.402 | 1.00 | 38.12 | O |
| ATOM | 6120 | N | LEU | R | 155 | 12.790 | 99.396 | 218.815 | 1.00 | 38.98 | N |
| ATOM | 6121 | CA | LEU | R | 155 | 12.021 | 98.194 | 219.147 | 1.00 | 39.81 | C |
| ATOM | 6123 | CB | LEU | R | 155 | 10.628 | 98.246 | 218.510 | 1.00 | 40.74 | C |
| ATOM | 6126 | CG | LEU | R | 155 | 9.645 | 97.113 | 218.842 | 1.00 | 41.26 | C |
| ATOM | 6128 | CD1 | LEU | R | 155 | 9.705 | 96.691 | 220.315 | 1.00 | 41.60 | C |
| ATOM | 6132 | CD2 | LEU | R | 155 | 8.228 | 97.534 | 218.471 | 1.00 | 41.43 | C |
| ATOM | 6136 | C | LEU | R | 155 | 12.751 | 96.929 | 218.713 | 1.00 | 39.74 | C |
| ATOM | 6137 | O | LEU | R | 155 | 12.812 | 95.960 | 219.468 | 1.00 | 39.19 | O |
| ATOM | 6139 | N | GLN | R | 156 | 13.294 | 96.950 | 217.494 | 1.00 | 41.34 | N |
| ATOM | 6140 | CA | GLN | R | 156 | 14.142 | 95.873 | 216.981 | 1.00 | 41.48 | C |
| ATOM | 6142 | CB | GLN | R | 156 | 13.400 | 95.061 | 215.913 | 1.00 | 42.42 | C |
| ATOM | 6145 | CG | GLN | R | 156 | 14.271 | 94.031 | 215.201 | 1.00 | 44.19 | C |
| ATOM | 6148 | CD | GLN | R | 156 | 13.467 | 92.985 | 214.451 | 1.00 | 44.98 | C |
| ATOM | 6149 | OE1 | GLN | R | 156 | 12.735 | 92.198 | 215.053 | 1.00 | 46.29 | O |
| ATOM | 6150 | NE2 | GLN | R | 156 | 13.614 | 92.960 | 213.131 | 1.00 | 48.64 | N |
| ATOM | 6153 | C | GLN | R | 156 | 15.430 | 96.463 | 216.394 | 1.00 | 40.01 | C |
| ATOM | 6154 | O | GLN | R | 156 | 15.367 | 97.361 | 215.551 | 1.00 | 37.09 | O |
| ATOM | 6156 | N | PRO | R | 157 | 16.604 | 95.965 | 216.834 | 1.00 | 39.73 | N |
| ATOM | 6157 | CA | PRO | R | 157 | 17.857 | 96.476 | 216.273 | 1.00 | 38.78 | C |
| ATOM | 6159 | CB | PRO | R | 157 | 18.945 | 95.755 | 217.086 | 1.00 | 39.02 | C |
| ATOM | 6162 | CG | PRO | R | 157 | 18.262 | 95.133 | 218.237 | 1.00 | 41.17 | C |
| ATOM | 6165 | CD | PRO | R | 157 | 16.839 | 94.925 | 217.851 | 1.00 | 40.99 | C |
| ATOM | 6168 | C | PRO | R | 157 | 18.020 | 96.146 | 214.790 | 1.00 | 37.84 | C |
| ATOM | 6169 | O | PRO | R | 157 | 17.398 | 95.208 | 214.286 | 1.00 | 35.63 | O |
| ATOM | 6170 | N | ALA | R | 158 | 18.859 | 96.917 | 214.106 | 1.00 | 38.62 | N |
| ATOM | 6171 | CA | ALA | R | 158 | 19.191 | 96.644 | 212.715 | 1.00 | 37.47 | C |
| ATOM | 6173 | CB | ALA | R | 158 | 19.820 | 97.871 | 212.066 | 1.00 | 38.14 | C |
| ATOM | 6177 | C | ALA | R | 158 | 20.143 | 95.458 | 212.628 | 1.00 | 37.70 | C |
| ATOM | 6178 | O | ALA | R | 158 | 20.823 | 95.115 | 213.600 | 1.00 | 37.32 | O |
| ATOM | 6180 | N | SER | R | 159 | 20.175 | 94.830 | 211.456 | 1.00 | 37.03 | N |
| ATOM | 6181 | CA | SER | R | 159 | 21.139 | 93.779 | 211.163 | 1.00 | 36.10 | C |
| ATOM | 6183 | CB | SER | R | 159 | 20.460 | 92.406 | 211.204 | 1.00 | 36.14 | C |
| ATOM | 6186 | OG | SER | R | 159 | 19.567 | 92.237 | 210.113 | 1.00 | 34.55 | O |
| ATOM | 6188 | C | SER | R | 159 | 21.738 | 94.046 | 209.787 | 1.00 | 35.53 | C |
| ATOM | 6189 | O | SER | R | 159 | 21.440 | 95.066 | 209.162 | 1.00 | 36.18 | O |
| ATOM | 6191 | N | ASN | R | 160 | 22.583 | 93.134 | 209.319 | 1.00 | 35.47 | N |
| ATOM | 6192 | CA | ASN | R | 160 | 23.112 | 93.212 | 207.955 | 1.00 | 34.24 | C |
| ATOM | 6194 | CB | ASN | R | 160 | 24.248 | 92.197 | 207.754 | 1.00 | 35.91 | C |
| ATOM | 6197 | CG | ASN | R | 160 | 23.792 | 90.754 | 207.929 | 1.00 | 38.15 | C |
| ATOM | 6198 | OD1 | ASN | R | 160 | 22.758 | 90.484 | 208.544 | 1.00 | 41.64 | O |
| ATOM | 6199 | ND2 | ASN | R | 160 | 24.568 | 89.819 | 207.390 | 1.00 | 41.23 | N |
| ATOM | 6202 | C | ASN | R | 160 | 22.020 | 93.024 | 206.890 | 1.00 | 31.94 | C |
| ATOM | 6203 | O | ASN | R | 160 | 22.188 | 93.455 | 205.749 | 1.00 | 32.45 | O |
| ATOM | 6205 | N | SER | R | 161 | 20.904 | 92.399 | 207.276 | 1.00 | 29.55 | N |
| ATOM | 6206 | CA | SER | R | 161 | 19.816 | 92.077 | 206.354 | 1.00 | 30.26 | C |
| ATOM | 6208 | CB | SER | R | 161 | 19.448 | 90.591 | 206.483 | 1.00 | 29.75 | C |
| ATOM | 6211 | OG | SER | R | 161 | 18.950 | 90.285 | 207.779 | 1.00 | 32.29 | O |
| ATOM | 6213 | C | SER | R | 161 | 18.551 | 92.929 | 206.524 | 1.00 | 29.66 | C |
| ATOM | 6214 | O | SER | R | 161 | 17.624 | 92.810 | 205.719 | 1.00 | 27.80 | O |
| ATOM | 6216 | N | SER | R | 162 | 18.495 | 93.773 | 207.558 | 1.00 | 32.80 | N |
| ATOM | 6217 | CA | SER | R | 162 | 17.269 | 94.525 | 207.853 | 1.00 | 31.16 | C |
| ATOM | 6219 | CB | SER | R | 162 | 16.289 | 93.641 | 208.626 | 1.00 | 32.12 | C |
| ATOM | 6222 | OG | SER | R | 162 | 16.816 | 93.267 | 209.888 | 1.00 | 32.67 | O |
| ATOM | 6224 | C | SER | R | 162 | 17.502 | 95.826 | 208.626 | 1.00 | 31.42 | C |
| ATOM | 6225 | O | SER | R | 162 | 18.462 | 95.955 | 209.389 | 1.00 | 29.65 | O |
| ATOM | 6227 | N | ASP | R | 163 | 16.602 | 96.783 | 208.417 | 1.00 | 30.71 | N |
| ATOM | 6228 | CA | ASP | R | 163 | 16.691 | 98.082 | 209.065 | 1.00 | 33.33 | C |
| ATOM | 6230 | CB | ASP | R | 163 | 15.872 | 99.122 | 208.285 | 1.00 | 34.54 | C |
| ATOM | 6233 | CG | ASP | R | 163 | 16.332 | 100.554 | 208.538 | 1.00 | 34.53 | C |
| ATOM | 6234 | OD1 | ASP | R | 163 | 17.245 | 100.772 | 209.365 | 1.00 | 33.92 | O |
| ATOM | 6235 | OD2 | ASP | R | 163 | 15.774 | 101.469 | 207.897 | 1.00 | 36.35 | O |
| ATOM | 6236 | C | ASP | R | 163 | 16.183 | 97.984 | 210.499 | 1.00 | 33.66 | C |
| ATOM | 6237 | O | ASP | R | 163 | 15.443 | 97.057 | 210.847 | 1.00 | 33.38 | O |
| ATOM | 6239 | N | ALA | R | 164 | 16.595 | 98.941 | 211.328 | 1.00 | 35.14 | N |
| ATOM | 6240 | CA | ALA | R | 164 | 16.078 | 99.056 | 212.689 | 1.00 | 34.23 | C |
| ATOM | 6242 | CB | ALA | R | 164 | 16.912 | 100.050 | 213.499 | 1.00 | 34.58 | C |
| ATOM | 6246 | C | ALA | R | 164 | 14.618 | 99.495 | 212.654 | 1.00 | 34.61 | C |
| ATOM | 6247 | O | ALA | R | 164 | 14.200 | 100.210 | 211.737 | 1.00 | 34.60 | O |
| ATOM | 6249 | N | ILE | R | 165 | 13.850 | 99.054 | 213.649 | 1.00 | 33.40 | N |
| ATOM | 6250 | CA | ILE | R | 165 | 12.448 | 99.434 | 213.791 | 1.00 | 34.04 | C |
| ATOM | 6252 | CB | ILE | R | 165 | 11.534 | 98.202 | 213.981 | 1.00 | 34.48 | C |
| ATOM | 6254 | CG1 | ILE | R | 165 | 11.845 | 97.130 | 212.930 | 1.00 | 34.62 | C |
| ATOM | 6257 | CD1 | ILE | R | 165 | 11.003 | 95.880 | 213.056 | 1.00 | 35.48 | C |
| ATOM | 6261 | CG2 | ILE | R | 165 | 10.057 | 98.604 | 213.900 | 1.00 | 34.33 | C |
| ATOM | 6265 | C | ILE | R | 165 | 12.322 | 100.355 | 215.004 | 1.00 | 33.30 | C |
| ATOM | 6266 | O | ILE | R | 165 | 12.823 | 100.038 | 216.084 | 1.00 | 30.10 | O |
| ATOM | 6268 | N | CYS | R | 166 | 11.661 | 101.495 | 214.813 | 1.00 | 34.70 | N |
| ATOM | 6269 | CA | CYS | R | 166 | 11.457 | 102.467 | 215.888 | 1.00 | 35.63 | C |
| ATOM | 6271 | CB | CYS | R | 166 | 11.245 | 103.867 | 215.306 | 1.00 | 37.75 | C |
| ATOM | 6274 | SG | CYS | R | 166 | 12.531 | 104.383 | 214.139 | 1.00 | 40.43 | S |
| ATOM | 6276 | C | CYS | R | 166 | 10.260 | 102.072 | 216.747 | 1.00 | 35.93 | C |
| ATOM | 6277 | O | CYS | R | 166 | 9.226 | 101.648 | 216.230 | 1.00 | 35.11 | O |
| ATOM | 6279 | N | LEU | T | 29 | 26.611 | 67.584 | 190.236 | 1.00 | 45.25 | N |
| ATOM | 6280 | CA | LEU | T | 29 | 26.448 | 68.732 | 191.185 | 1.00 | 45.04 | C |
| ATOM | 6282 | CB | LEU | T | 29 | 24.963 | 69.084 | 191.357 | 1.00 | 44.44 | C |
| ATOM | 6285 | CG | LEU | T | 29 | 24.607 | 70.255 | 192.280 | 1.00 | 44.76 | C |
| ATOM | 6287 | CD1 | LEU | T | 29 | 25.231 | 71.553 | 191.792 | 1.00 | 45.26 | C |
| ATOM | 6291 | CD2 | LEU | T | 29 | 23.092 | 70.390 | 192.386 | 1.00 | 45.14 | C |
| ATOM | 6295 | C | LEU | T | 29 | 27.073 | 68.421 | 192.546 | 1.00 | 44.26 | C |
| ATOM | 6296 | O | LEU | T | 29 | 26.975 | 67.297 | 193.037 | 1.00 | 45.64 | O |
| ATOM | 6300 | N | HIS | T | 30 | 27.719 | 69.422 | 193.137 | 1.00 | 44.57 | N |
| ATOM | 6301 | CA | HIS | T | 30 | 28.322 | 69.299 | 194.462 | 1.00 | 43.88 | C |
| ATOM | 6303 | CB | HIS | T | 30 | 29.793 | 68.900 | 194.339 | 1.00 | 46.90 | C |
| ATOM | 6306 | CG | HIS | T | 30 | 30.511 | 68.812 | 195.651 | 1.00 | 47.92 | C |
| ATOM | 6307 | ND1 | HIS | T | 30 | 31.710 | 69.450 | 195.888 | 1.00 | 48.91 | N |
| ATOM | 6309 | CE1 | HIS | T | 30 | 32.104 | 69.197 | 197.124 | 1.00 | 49.55 | C |
| ATOM | 6311 | NE2 | HIS | T | 30 | 31.201 | 68.424 | 197.699 | 1.00 | 50.77 | N |
| ATOM | 6313 | CD2 | HIS | T | 30 | 30.193 | 68.169 | 196.800 | 1.00 | 49.47 | C |
| ATOM | 6315 | C | HIS | T | 30 | 28.193 | 70.621 | 195.222 | 1.00 | 43.43 | C |
| ATOM | 6316 | O | HIS | T | 30 | 28.754 | 71.638 | 194.807 | 1.00 | 44.57 | O |
| ATOM | 6318 | N | CYS | T | 31 | 27.442 | 70.599 | 196.322 | 1.00 | 39.30 | N |
| ATOM | 6319 | CA | CYS | T | 31 | 27.268 | 71.770 | 197.175 | 1.00 | 38.29 | C |
| ATOM | 6321 | CB | CYS | T | 31 | 25.784 | 72.149 | 197.288 | 1.00 | 34.77 | C |
| ATOM | 6324 | SG | CYS | T | 31 | 24.952 | 72.541 | 195.709 | 1.00 | 32.97 | S |
| ATOM | 6326 | C | CYS | T | 31 | 27.854 | 71.472 | 198.558 | 1.00 | 38.30 | C |
| ATOM | 6327 | O | CYS | T | 31 | 27.789 | 70.339 | 199.039 | 1.00 | 37.06 | O |
| ATOM | 6329 | N | VAL | T | 32 | 28.432 | 72.494 | 199.183 | 1.00 | 39.22 | N |
| ATOM | 6330 | CA | VAL | T | 32 | 29.041 | 72.368 | 200.510 | 1.00 | 39.62 | C |
| ATOM | 6332 | CB | VAL | T | 32 | 30.595 | 72.332 | 200.437 | 1.00 | 40.98 | C |
| ATOM | 6334 | CG1 | VAL | T | 32 | 31.075 | 71.051 | 199.762 | 1.00 | 40.96 | C |
| ATOM | 6338 | CG2 | VAL | T | 32 | 31.147 | 73.564 | 199.715 | 1.00 | 41.68 | C |
| ATOM | 6342 | C | VAL | T | 32 | 28.611 | 73.528 | 201.407 | 1.00 | 40.80 | C |
| ATOM | 6343 | O | VAL | T | 32 | 27.970 | 74.478 | 200.945 | 1.00 | 41.97 | O |
| ATOM | 6345 | N | GLY | T | 33 | 28.962 | 73.433 | 202.688 | 1.00 | 38.89 | N |
| ATOM | 6346 | CA | GLY | T | 33 | 28.724 | 74.509 | 203.643 | 1.00 | 37.81 | C |
| ATOM | 6349 | C | GLY | T | 33 | 27.253 | 74.820 | 203.861 | 1.00 | 37.92 | C |
| ATOM | 6350 | O | GLY | T | 33 | 26.423 | 73.911 | 203.998 | 1.00 | 37.49 | O |
| ATOM | 6352 | N | ASP | T | 34 | 26.926 | 76.110 | 203.879 | 1.00 | 34.95 | N |
| ATOM | 6353 | CA | ASP | T | 34 | 25.574 | 76.563 | 204.203 | 1.00 | 33.25 | C |
| ATOM | 6355 | CB | ASP | T | 34 | 25.639 | 77.883 | 204.982 | 1.00 | 34.65 | C |
| ATOM | 6358 | CG | ASP | T | 34 | 26.369 | 77.744 | 206.318 | 1.00 | 37.07 | C |
| ATOM | 6359 | OD1 | ASP | T | 34 | 26.282 | 76.669 | 206.952 | 1.00 | 35.96 | O |
| ATOM | 6360 | OD2 | ASP | T | 34 | 27.031 | 78.716 | 206.735 | 1.00 | 39.48 | O |
| ATOM | 6361 | C | ASP | T | 34 | 24.719 | 76.717 | 202.945 | 1.00 | 30.58 | C |
| ATOM | 6362 | O | ASP | T | 34 | 23.966 | 77.685 | 202.807 | 1.00 | 29.05 | O |
| ATOM | 6364 | N | THR | T | 35 | 24.834 | 75.751 | 202.036 | 1.00 | 28.25 | N |
| ATOM | 6365 | CA | THR | T | 35 | 24.065 | 75.755 | 200.800 | 1.00 | 30.36 | C |
| ATOM | 6367 | CB | THR | T | 35 | 24.968 | 75.983 | 199.566 | 1.00 | 29.95 | C |
| ATOM | 6369 | OG1 | THR | T | 35 | 25.861 | 74.876 | 199.409 | 1.00 | 30.62 | O |
| ATOM | 6371 | CG2 | THR | T | 35 | 25.776 | 77.280 | 199.704 | 1.00 | 31.09 | C |
| ATOM | 6375 | C | THR | T | 35 | 23.313 | 74.440 | 200.618 | 1.00 | 27.31 | C |
| ATOM | 6376 | O | THR | T | 35 | 23.570 | 73.458 | 201.322 | 1.00 | 26.74 | O |
| ATOM | 6378 | N | TYR | T | 36 | 22.381 | 74.437 | 199.671 | 1.00 | 25.45 | N |
| ATOM | 6379 | CA | TYR | T | 36 | 21.630 | 73.232 | 199.321 | 1.00 | 28.04 | C |
| ATOM | 6381 | CB | TYR | T | 36 | 20.255 | 73.204 | 200.011 | 1.00 | 27.81 | C |
| ATOM | 6384 | CG | TYR | T | 36 | 19.340 | 74.349 | 199.639 | 1.00 | 27.67 | C |
| ATOM | 6385 | CD1 | TYR | T | 36 | 19.443 | 75.576 | 200.277 | 1.00 | 29.75 | C |
| ATOM | 6387 | CE1 | TYR | T | 36 | 18.614 | 76.637 | 199.937 | 1.00 | 29.46 | C |
| ATOM | 6389 | CZ | TYR | T | 36 | 17.673 | 76.474 | 198.948 | 1.00 | 27.01 | C |
| ATOM | 6390 | OH | TYR | T | 36 | 16.863 | 77.531 | 198.621 | 1.00 | 28.38 | O |
| ATOM | 6392 | CE2 | TYR | T | 36 | 17.544 | 75.264 | 198.299 | 1.00 | 26.11 | C |
| ATOM | 6394 | CD2 | TYR | T | 36 | 18.379 | 74.206 | 198.643 | 1.00 | 27.30 | C |
| ATOM | 6396 | C | TYR | T | 36 | 21.502 | 73.118 | 197.799 | 1.00 | 26.04 | C |
| ATOM | 6397 | O | TYR | T | 36 | 21.409 | 74.130 | 197.102 | 1.00 | 25.06 | O |
| ATOM | 6399 | N | PRO | T | 37 | 21.549 | 71.883 | 197.274 | 1.00 | 27.52 | N |
| ATOM | 6400 | CA | PRO | T | 37 | 21.453 | 71.632 | 195.839 | 1.00 | 27.78 | C |
| ATOM | 6402 | CB | PRO | T | 37 | 22.022 | 70.216 | 195.704 | 1.00 | 28.36 | C |
| ATOM | 6405 | CG | PRO | T | 37 | 21.717 | 69.568 | 197.000 | 1.00 | 26.84 | C |
| ATOM | 6408 | CD | PRO | T | 37 | 21.760 | 70.638 | 198.040 | 1.00 | 27.31 | C |
| ATOM | 6411 | C | PRO | T | 37 | 20.025 | 71.695 | 195.289 | 1.00 | 28.89 | C |
| ATOM | 6412 | O | PRO | T | 37 | 19.117 | 71.068 | 195.843 | 1.00 | 28.47 | O |
| ATOM | 6413 | N | SER | T | 38 | 19.835 | 72.441 | 194.204 | 1.00 | 28.14 | N |
| ATOM | 6414 | CA | SER | T | 38 | 18.529 | 72.528 | 193.561 | 1.00 | 30.24 | C |
| ATOM | 6416 | CB | SER | T | 38 | 17.629 | 73.500 | 194.337 | 1.00 | 31.57 | C |
| ATOM | 6419 | OG | SER | T | 38 | 16.276 | 73.400 | 193.930 | 1.00 | 30.17 | O |
| ATOM | 6421 | C | SER | T | 38 | 18.672 | 72.974 | 192.109 | 1.00 | 30.46 | C |
| ATOM | 6422 | O | SER | T | 38 | 19.402 | 73.919 | 191.821 | 1.00 | 29.55 | O |
| ATOM | 6424 | N | ASN | T | 39 | 17.988 | 72.274 | 191.201 | 1.00 | 28.90 | N |
| ATOM | 6425 | CA | ASN | T | 39 | 17.940 | 72.632 | 189.783 | 1.00 | 30.97 | C |
| ATOM | 6427 | CB | ASN | T | 39 | 17.090 | 73.897 | 189.593 | 1.00 | 32.78 | C |
| ATOM | 6430 | CG | ASN | T | 39 | 15.858 | 73.911 | 190.478 | 1.00 | 30.17 | C |
| ATOM | 6431 | OD1 | ASN | T | 39 | 14.903 | 73.160 | 190.256 | 1.00 | 32.83 | O |
| ATOM | 6432 | ND2 | ASN | T | 39 | 15.881 | 74.750 | 191.505 | 1.00 | 25.86 | N |
| ATOM | 6435 | C | ASN | T | 39 | 19.336 | 72.798 | 189.152 | 1.00 | 31.10 | C |
| ATOM | 6436 | O | ASN | T | 39 | 19.575 | 73.724 | 188.380 | 1.00 | 35.41 | O |
| ATOM | 6438 | N | ASP | T | 40 | 20.242 | 71.888 | 189.506 | 1.00 | 32.75 | N |
| ATOM | 6439 | CA | ASP | T | 40 | 21.613 | 71.837 | 188.980 | 1.00 | 32.55 | C |
| ATOM | 6441 | CB | ASP | T | 40 | 21.611 | 71.695 | 187.447 | 1.00 | 36.00 | C |
| ATOM | 6444 | CG | ASP | T | 40 | 22.965 | 71.254 | 186.891 | 1.00 | 39.64 | C |
| ATOM | 6445 | OD1 | ASP | T | 40 | 23.617 | 70.376 | 187.499 | 1.00 | 43.86 | O |
| ATOM | 6446 | OD2 | ASP | T | 40 | 23.381 | 71.788 | 185.838 | 1.00 | 47.17 | O |
| ATOM | 6447 | C | ASP | T | 40 | 22.493 | 73.022 | 189.406 | 1.00 | 31.39 | C |
| ATOM | 6448 | O | ASP | T | 40 | 23.466 | 73.346 | 188.726 | 1.00 | 29.21 | O |
| ATOM | 6450 | N | ARG | T | 41 | 22.152 | 73.658 | 190.526 | 1.00 | 29.86 | N |
| ATOM | 6451 | CA | ARG | T | 41 | 22.999 | 74.704 | 191.112 | 1.00 | 29.44 | C |
| ATOM | 6453 | CB | ARG | T | 41 | 22.717 | 76.087 | 190.481 | 1.00 | 27.78 | C |
| ATOM | 6456 | CG | ARG | T | 41 | 21.272 | 76.583 | 190.522 | 1.00 | 28.43 | C |
| ATOM | 6459 | CD | ARG | T | 41 | 20.885 | 77.182 | 191.874 | 1.00 | 26.43 | C |
| ATOM | 6462 | NE | ARG | T | 41 | 19.823 | 78.181 | 191.763 | 1.00 | 25.92 | N |
| ATOM | 6464 | CZ | ARG | T | 41 | 18.527 | 77.901 | 191.643 | 1.00 | 26.98 | C |
| ATOM | 6465 | NH1 | ARG | T | 41 | 18.111 | 76.647 | 191.629 | 1.00 | 26.55 | N |
| ATOM | 6468 | NH2 | ARG | T | 41 | 17.634 | 78.881 | 191.550 | 1.00 | 27.51 | N |
| ATOM | 6471 | C | ARG | T | 41 | 22.867 | 74.720 | 192.637 | 1.00 | 27.67 | C |
| ATOM | 6472 | O | ARG | T | 41 | 22.093 | 73.950 | 193.211 | 1.00 | 27.15 | O |
| ATOM | 6474 | N | CYS | T | 42 | 23.657 | 75.565 | 193.291 | 1.00 | 27.93 | N |
| ATOM | 6475 | CA | CYS | T | 42 | 23.658 | 75.639 | 194.748 | 1.00 | 28.67 | C |
| ATOM | 6477 | CB | CYS | T | 42 | 25.095 | 75.597 | 195.280 | 1.00 | 29.86 | C |
| ATOM | 6480 | SG | CYS | T | 42 | 26.032 | 74.072 | 194.871 | 1.00 | 32.16 | S |
| ATOM | 6482 | C | CYS | T | 42 | 22.939 | 76.906 | 195.216 | 1.00 | 28.39 | C |
| ATOM | 6483 | O | CYS | T | 42 | 23.104 | 77.984 | 194.635 | 1.00 | 27.40 | O |
| ATOM | 6485 | N | CYS | T | 43 | 22.144 | 76.761 | 196.272 | 1.00 | 28.42 | N |
| ATOM | 6486 | CA | CYS | T | 43 | 21.329 | 77.851 | 196.807 | 1.00 | 28.41 | C |
| ATOM | 6488 | CB | CYS | T | 43 | 19.851 | 77.498 | 196.676 | 1.00 | 33.18 | C |
| ATOM | 6491 | SG | CYS | T | 43 | 19.315 | 77.307 | 194.956 | 1.00 | 34.22 | S |
| ATOM | 6493 | C | CYS | T | 43 | 21.697 | 78.114 | 198.265 | 1.00 | 26.97 | C |
| ATOM | 6494 | O | CYS | T | 43 | 22.092 | 77.201 | 198.987 | 1.00 | 26.47 | O |
| ATOM | 6496 | N | HIS | T | 44 | 21.587 | 79.366 | 198.688 | 1.00 | 27.05 | N |
| ATOM | 6497 | CA | HIS | T | 44 | 21.975 | 79.750 | 200.039 | 1.00 | 28.91 | C |
| ATOM | 6499 | CB | HIS | T | 44 | 22.377 | 81.220 | 200.076 | 1.00 | 27.22 | C |
| ATOM | 6502 | CG | HIS | T | 44 | 23.637 | 81.520 | 199.326 | 1.00 | 24.93 | C |
| ATOM | 6503 | ND1 | HIS | T | 44 | 24.871 | 81.070 | 199.741 | 1.00 | 25.54 | N |
| ATOM | 6505 | CE1 | HIS | T | 44 | 25.798 | 81.491 | 198.899 | 1.00 | 27.28 | C |
| ATOM | 6507 | NE2 | HIS | T | 44 | 25.208 | 82.201 | 197.953 | 1.00 | 25.52 | N |
| ATOM | 6509 | CD2 | HIS | T | 44 | 23.856 | 82.232 | 198.196 | 1.00 | 23.47 | C |
| ATOM | 6511 | C | HIS | T | 44 | 20.857 | 79.501 | 201.056 | 1.00 | 29.95 | C |
| ATOM | 6512 | O | HIS | T | 44 | 19.704 | 79.863 | 200.823 | 1.00 | 29.36 | O |
| ATOM | 6514 | N | GLU | T | 45 | 21.217 | 78.877 | 202.176 | 1.00 | 29.90 | N |
| ATOM | 6515 | CA | GLU | T | 45 | 20.345 | 78.803 | 203.347 | 1.00 | 34.29 | C |
| ATOM | 6517 | CB | GLU | T | 45 | 20.865 | 77.765 | 204.342 | 1.00 | 35.37 | C |
| ATOM | 6520 | CG | GLU | T | 45 | 20.852 | 76.343 | 203.817 | 1.00 | 37.60 | C |
| ATOM | 6523 | CD | GLU | T | 45 | 21.077 | 75.328 | 204.917 | 1.00 | 39.59 | C |
| ATOM | 6524 | OE1 | GLU | T | 45 | 22.219 | 75.234 | 205.416 | 1.00 | 42.58 | O |
| ATOM | 6525 | OE2 | GLU | T | 45 | 20.108 | 74.627 | 205.286 | 1.00 | 43.53 | O |
| ATOM | 6526 | C | GLU | T | 45 | 20.281 | 80.168 | 204.030 | 1.00 | 33.27 | C |
| ATOM | 6527 | O | GLU | T | 45 | 21.146 | 81.018 | 203.813 | 1.00 | 31.18 | O |
| ATOM | 6529 | N | CYS | T | 46 | 19.260 | 80.368 | 204.859 | 1.00 | 33.32 | N |
| ATOM | 6530 | CA | CYS | T | 46 | 19.086 | 81.627 | 205.579 | 1.00 | 37.24 | C |
| ATOM | 6532 | CB | CYS | T | 46 | 17.669 | 81.746 | 206.147 | 1.00 | 37.21 | C |
| ATOM | 6535 | SG | CYS | T | 46 | 16.330 | 81.458 | 204.962 | 1.00 | 40.81 | S |
| ATOM | 6537 | C | CYS | T | 46 | 20.108 | 81.748 | 206.713 | 1.00 | 38.88 | C |
| ATOM | 6538 | O | CYS | T | 46 | 20.474 | 80.755 | 207.346 | 1.00 | 39.39 | O |
| ATOM | 6540 | N | ARG | T | 47 | 20.557 | 82.972 | 206.964 | 1.00 | 41.13 | N |
| ATOM | 6541 | CA | ARG | T | 47 | 21.533 | 83.241 | 208.019 | 1.00 | 42.38 | C |
| ATOM | 6543 | CB | ARG | T | 47 | 22.393 | 84.455 | 207.636 | 1.00 | 49.03 | C |
| ATOM | 6546 | CG | ARG | T | 47 | 23.272 | 84.232 | 206.404 | 1.00 | 51.61 | C |
| ATOM | 6549 | CD | ARG | T | 47 | 24.320 | 83.155 | 206.663 | 1.00 | 55.11 | C |
| ATOM | 6552 | NE | ARG | T | 47 | 25.277 | 83.021 | 205.567 | 1.00 | 56.09 | N |
| ATOM | 6554 | CZ | ARG | T | 47 | 26.427 | 82.349 | 205.640 | 1.00 | 59.65 | C |
| ATOM | 6555 | NH1 | ARG | T | 47 | 26.789 | 81.736 | 206.766 | 1.00 | 60.81 | N |
| ATOM | 6558 | NH2 | ARG | T | 47 | 27.231 | 82.293 | 204.581 | 1.00 | 59.12 | N |
| ATOM | 6561 | C | ARG | T | 47 | 20.811 | 83.483 | 209.344 | 1.00 | 41.54 | C |
| ATOM | 6562 | O | ARG | T | 47 | 19.590 | 83.637 | 209.360 | 1.00 | 38.49 | O |
| ATOM | 6564 | N | PRO | T | 48 | 21.558 | 83.490 | 210.467 | 1.00 | 41.28 | N |
| ATOM | 6565 | CA | PRO | T | 48 | 20.964 | 83.901 | 211.739 | 1.00 | 39.71 | C |
| ATOM | 6567 | CB | PRO | T | 48 | 22.177 | 84.027 | 212.663 | 1.00 | 41.04 | C |
| ATOM | 6570 | CG | PRO | T | 48 | 23.155 | 83.055 | 212.118 | 1.00 | 40.74 | C |
| ATOM | 6573 | CD | PRO | T | 48 | 22.967 | 83.082 | 210.631 | 1.00 | 40.28 | C |
| ATOM | 6576 | C | PRO | T | 48 | 20.227 | 85.235 | 211.624 | 1.00 | 37.36 | C |
| ATOM | 6577 | O | PRO | T | 48 | 20.698 | 86.145 | 210.945 | 1.00 | 33.57 | O |
| ATOM | 6578 | N | GLY | T | 49 | 19.072 | 85.334 | 212.272 | 1.00 | 36.81 | N |
| ATOM | 6579 | CA | GLY | T | 49 | 18.249 | 86.537 | 212.195 | 1.00 | 37.64 | C |
| ATOM | 6582 | C | GLY | T | 49 | 17.273 | 86.537 | 211.036 | 1.00 | 36.52 | C |
| ATOM | 6583 | O | GLY | T | 49 | 16.550 | 87.514 | 210.842 | 1.00 | 36.20 | O |
| ATOM | 6585 | N | ASN | T | 50 | 17.251 | 85.447 | 210.266 | 1.00 | 35.60 | N |
| ATOM | 6586 | CA | ASN | T | 50 | 16.338 | 85.296 | 209.131 | 1.00 | 37.14 | C |
| ATOM | 6588 | CB | ASN | T | 50 | 17.052 | 85.604 | 207.808 | 1.00 | 38.58 | C |
| ATOM | 6591 | CG | ASN | T | 50 | 17.710 | 86.975 | 207.793 | 1.00 | 38.00 | C |
| ATOM | 6592 | OD1 | ASN | T | 50 | 18.847 | 87.132 | 208.237 | 1.00 | 39.14 | O |
| ATOM | 6593 | ND2 | ASN | T | 50 | 17.009 | 87.967 | 207.252 | 1.00 | 37.25 | N |
| ATOM | 6596 | C | ASN | T | 50 | 15.762 | 83.878 | 209.064 | 1.00 | 35.64 | C |
| ATOM | 6597 | O | ASN | T | 50 | 16.420 | 82.918 | 209.467 | 1.00 | 34.45 | O |
| ATOM | 6599 | N | GLY | T | 51 | 14.539 | 83.758 | 208.551 | 1.00 | 35.93 | N |
| ATOM | 6600 | CA | GLY | T | 51 | 13.888 | 82.457 | 208.354 | 1.00 | 34.79 | C |
| ATOM | 6603 | C | GLY | T | 51 | 13.483 | 82.259 | 206.902 | 1.00 | 32.95 | C |
| ATOM | 6604 | O | GLY | T | 51 | 13.227 | 83.229 | 206.192 | 1.00 | 32.70 | O |
| ATOM | 6606 | N | MET | T | 52 | 13.418 | 81.008 | 206.455 | 1.00 | 33.33 | N |
| ATOM | 6607 | CA | MET | T | 52 | 13.084 | 80.732 | 205.052 | 1.00 | 35.61 | C |
| ATOM | 6609 | CB | MET | T | 52 | 13.680 | 79.413 | 204.565 | 1.00 | 39.08 | C |
| ATOM | 6612 | CG | MET | T | 52 | 13.834 | 79.383 | 203.029 | 1.00 | 37.86 | C |
| ATOM | 6615 | SD | MET | T | 52 | 14.739 | 77.961 | 202.481 | 1.00 | 45.92 | S |
| ATOM | 6616 | CE | MET | T | 52 | 16.420 | 78.412 | 202.871 | 1.00 | 40.41 | C |
| ATOM | 6620 | C | MET | T | 52 | 11.587 | 80.729 | 204.781 | 1.00 | 32.52 | C |
| ATOM | 6621 | O | MET | T | 52 | 10.804 | 80.094 | 205.496 | 1.00 | 29.20 | O |
| ATOM | 6623 | N | VAL | T | 53 | 11.209 | 81.443 | 203.725 | 1.00 | 30.65 | N |
| ATOM | 6624 | CA | VAL | T | 53 | 9.843 | 81.453 | 203.219 | 1.00 | 29.99 | C |
| ATOM | 6626 | CB | VAL | T | 53 | 9.471 | 82.846 | 202.684 | 1.00 | 29.17 | C |
| ATOM | 6628 | CG1 | VAL | T | 53 | 8.065 | 82.852 | 202.098 | 1.00 | 29.90 | C |
| ATOM | 6632 | CG2 | VAL | T | 53 | 9.606 | 83.886 | 203.799 | 1.00 | 29.24 | C |
| ATOM | 6636 | C | VAL | T | 53 | 9.708 | 80.423 | 202.100 | 1.00 | 30.26 | C |
| ATOM | 6637 | O | VAL | T | 53 | 8.728 | 79.684 | 202.036 | 1.00 | 27.00 | O |
| ATOM | 6639 | N | SER | T | 54 | 10.698 | 80.375 | 201.216 | 1.00 | 28.75 | N |
| ATOM | 6640 | CA | SER | T | 54 | 10.643 | 79.470 | 200.082 | 1.00 | 28.26 | C |
| ATOM | 6642 | CB | SER | T | 54 | 9.778 | 80.064 | 198.958 | 1.00 | 29.61 | C |
| ATOM | 6645 | OG | SER | T | 54 | 10.352 | 81.247 | 198.429 | 1.00 | 28.78 | O |
| ATOM | 6647 | C | SER | T | 54 | 12.037 | 79.156 | 199.570 | 1.00 | 29.71 | C |
| ATOM | 6648 | O | SER | T | 54 | 12.883 | 80.044 | 199.465 | 1.00 | 28.78 | O |
| ATOM | 6650 | N | ARG | T | 55 | 12.268 | 77.883 | 199.258 | 1.00 | 32.04 | N |
| ATOM | 6651 | CA | ARG | T | 55 | 13.519 | 77.460 | 198.657 | 1.00 | 29.82 | C |
| ATOM | 6653 | CB | ARG | T | 55 | 13.635 | 75.940 | 198.678 | 1.00 | 29.36 | C |
| ATOM | 6656 | CG | ARG | T | 55 | 13.856 | 75.350 | 200.060 | 1.00 | 29.40 | C |
| ATOM | 6659 | CD | ARG | T | 55 | 14.373 | 73.956 | 199.936 | 1.00 | 30.06 | C |
| ATOM | 6662 | NE | ARG | T | 55 | 14.554 | 73.290 | 201.221 | 1.00 | 30.89 | N |
| ATOM | 6664 | CZ | ARG | T | 55 | 15.646 | 73.367 | 201.982 | 1.00 | 30.59 | C |
| ATOM | 6665 | NH1 | ARG | T | 55 | 16.699 | 74.082 | 201.610 | 1.00 | 30.30 | N |
| ATOM | 6668 | NH2 | ARG | T | 55 | 15.689 | 72.694 | 203.125 | 1.00 | 29.56 | N |
| ATOM | 6671 | C | ARG | T | 55 | 13.590 | 77.956 | 197.220 | 1.00 | 28.31 | C |
| ATOM | 6672 | O | ARG | T | 55 | 12.567 | 78.292 | 196.616 | 1.00 | 26.70 | O |
| ATOM | 6674 | N | CYS | T | 56 | 14.800 | 77.999 | 196.674 | 1.00 | 30.25 | N |
| ATOM | 6675 | CA | CYS | T | 56 | 14.984 | 78.367 | 195.277 | 1.00 | 30.06 | C |
| ATOM | 6677 | CB | CYS | T | 56 | 16.476 | 78.454 | 194.923 | 1.00 | 31.60 | C |
| ATOM | 6680 | SG | CYS | T | 56 | 17.323 | 76.877 | 195.055 | 1.00 | 33.41 | S |
| ATOM | 6682 | C | CYS | T | 56 | 14.263 | 77.360 | 194.373 | 1.00 | 31.78 | C |
| ATOM | 6683 | O | CYS | T | 56 | 13.838 | 76.279 | 194.811 | 1.00 | 34.15 | O |
| ATOM | 6685 | N | SER | T | 57 | 14.077 | 77.750 | 193.122 | 1.00 | 31.02 | N |
| ATOM | 6686 | CA | SER | T | 57 | 13.426 | 76.901 | 192.133 | 1.00 | 32.12 | C |
| ATOM | 6688 | CB | SER | T | 57 | 11.931 | 77.217 | 192.061 | 1.00 | 32.64 | C |
| ATOM | 6691 | OG | SER | T | 57 | 11.707 | 78.532 | 191.595 | 1.00 | 33.86 | O |
| ATOM | 6693 | C | SER | T | 57 | 14.126 | 77.125 | 190.796 | 1.00 | 32.36 | C |
| ATOM | 6694 | O | SER | T | 57 | 15.181 | 77.756 | 190.754 | 1.00 | 30.27 | O |
| ATOM | 6696 | N | ARG | T | 58 | 13.567 | 76.592 | 189.716 | 1.00 | 34.30 | N |
| ATOM | 6697 | CA | ARG | T | 58 | 14.218 | 76.670 | 188.414 | 1.00 | 35.58 | C |
| ATOM | 6699 | CB | ARG | T | 58 | 13.404 | 75.912 | 187.357 | 1.00 | 38.66 | C |
| ATOM | 6702 | CG | ARG | T | 58 | 14.107 | 75.847 | 186.009 | 1.00 | 42.37 | C |
| ATOM | 6705 | CD | ARG | T | 58 | 13.631 | 74.685 | 185.151 | 1.00 | 47.41 | C |
| ATOM | 6708 | NE | ARG | T | 58 | 12.320 | 74.925 | 184.554 | 1.00 | 51.07 | N |
| ATOM | 6710 | CZ | ARG | T | 58 | 11.786 | 74.188 | 183.577 | 1.00 | 54.07 | C |
| ATOM | 6711 | NH1 | ARG | T | 58 | 12.443 | 73.146 | 183.066 | 1.00 | 54.39 | N |
| ATOM | 6714 | NH2 | ARG | T | 58 | 10.584 | 74.495 | 183.101 | 1.00 | 53.65 | N |
| ATOM | 6717 | C | ARG | T | 58 | 14.472 | 78.111 | 187.957 | 1.00 | 32.18 | C |
| ATOM | 6718 | O | ARG | T | 58 | 15.482 | 78.383 | 187.312 | 1.00 | 32.39 | O |
| ATOM | 6720 | N | SER | T | 59 | 13.564 | 79.021 | 188.294 | 1.00 | 29.54 | N |
| ATOM | 6721 | CA | SER | T | 59 | 13.653 | 80.415 | 187.843 | 1.00 | 32.97 | C |
| ATOM | 6723 | CB | SER | T | 59 | 12.607 | 80.663 | 186.751 | 1.00 | 38.07 | C |
| ATOM | 6726 | OG | SER | T | 59 | 12.872 | 79.848 | 185.621 | 1.00 | 41.86 | O |
| ATOM | 6728 | C | SER | T | 59 | 13.486 | 81.444 | 188.970 | 1.00 | 29.09 | C |
| ATOM | 6729 | O | SER | T | 59 | 13.271 | 82.626 | 188.700 | 1.00 | 28.44 | O |
| ATOM | 6731 | N | GLN | T | 60 | 13.600 | 81.004 | 190.222 | 1.00 | 30.48 | N |
| ATOM | 6732 | CA | GLN | T | 60 | 13.478 | 81.898 | 191.372 | 1.00 | 29.22 | C |
| ATOM | 6734 | CB | GLN | T | 60 | 12.106 | 81.748 | 192.049 | 1.00 | 33.69 | C |
| ATOM | 6737 | CG | GLN | T | 60 | 10.894 | 81.963 | 191.133 | 1.00 | 37.47 | C |
| ATOM | 6740 | CD | GLN | T | 60 | 10.779 | 83.388 | 190.608 | 1.00 | 40.36 | C |
| ATOM | 6741 | OE1 | GLN | T | 60 | 11.082 | 84.352 | 191.312 | 1.00 | 40.60 | O |
| ATOM | 6742 | NE2 | GLN | T | 60 | 10.332 | 83.523 | 189.362 | 1.00 | 40.94 | N |
| ATOM | 6745 | C | GLN | T | 60 | 14.570 | 81.621 | 192.398 | 1.00 | 26.24 | C |
| ATOM | 6746 | O | GLN | T | 60 | 15.021 | 80.476 | 192.553 | 1.00 | 27.04 | O |
| ATOM | 6748 | N | ASN | T | 61 | 14.978 | 82.679 | 193.095 | 1.00 | 24.72 | N |
| ATOM | 6749 | CA | ASN | T | 61 | 15.935 | 82.593 | 194.194 | 1.00 | 27.28 | C |
| ATOM | 6751 | CB | ASN | T | 61 | 16.594 | 83.958 | 194.457 | 1.00 | 25.19 | C |
| ATOM | 6754 | CG | ASN | T | 61 | 17.578 | 84.365 | 193.366 | 1.00 | 24.89 | C |
| ATOM | 6755 | OD1 | ASN | T | 61 | 17.427 | 85.410 | 192.734 | 1.00 | 22.67 | O |
| ATOM | 6756 | ND2 | ASN | T | 61 | 18.595 | 83.544 | 193.150 | 1.00 | 25.92 | N |
| ATOM | 6759 | C | ASN | T | 61 | 15.275 | 82.131 | 195.489 | 1.00 | 29.63 | C |
| ATOM | 6760 | O | ASN | T | 61 | 14.054 | 82.209 | 195.644 | 1.00 | 26.77 | O |
| ATOM | 6762 | N | THR | T | 62 | 16.104 | 81.666 | 196.418 | 1.00 | 31.32 | N |
| ATOM | 6763 | CA | THR | T | 62 | 15.687 | 81.435 | 197.801 | 1.00 | 30.06 | C |
| ATOM | 6765 | CB | THR | T | 62 | 16.907 | 81.062 | 198.675 | 1.00 | 30.83 | C |
| ATOM | 6767 | OG1 | THR | T | 62 | 17.582 | 79.939 | 198.098 | 1.00 | 30.36 | O |
| ATOM | 6769 | CG2 | THR | T | 62 | 16.489 | 80.730 | 200.105 | 1.00 | 32.37 | C |
| ATOM | 6773 | C | THR | T | 62 | 15.082 | 82.716 | 198.373 | 1.00 | 32.30 | C |
| ATOM | 6774 | O | THR | T | 62 | 15.616 | 83.802 | 198.141 | 1.00 | 27.58 | O |
| ATOM | 6776 | N | VAL | T | 63 | 13.979 | 82.588 | 199.114 | 1.00 | 29.43 | N |
| ATOM | 6777 | CA | VAL | T | 63 | 13.396 | 83.728 | 199.830 | 1.00 | 32.45 | C |
| ATOM | 6779 | CB | VAL | T | 63 | 11.912 | 83.971 | 199.454 | 1.00 | 31.19 | C |
| ATOM | 6781 | CG1 | VAL | T | 63 | 11.348 | 85.161 | 200.222 | 1.00 | 32.49 | C |
| ATOM | 6785 | CG2 | VAL | T | 63 | 11.773 | 84.208 | 197.957 | 1.00 | 31.55 | C |
| ATOM | 6789 | C | VAL | T | 63 | 13.515 | 83.528 | 201.336 | 1.00 | 33.39 | C |
| ATOM | 6790 | O | VAL | T | 63 | 12.864 | 82.647 | 201.916 | 1.00 | 30.03 | O |
| ATOM | 6792 | N | CYS | T | 64 | 14.372 | 84.340 | 201.952 | 1.00 | 34.36 | N |
| ATOM | 6793 | CA | CYS | T | 64 | 14.486 | 84.426 | 203.402 | 1.00 | 37.87 | C |
| ATOM | 6795 | CB | CYS | T | 64 | 15.941 | 84.263 | 203.846 | 1.00 | 39.41 | C |
| ATOM | 6798 | SG | CYS | T | 64 | 16.694 | 82.686 | 203.382 | 1.00 | 41.51 | S |
| ATOM | 6800 | C | CYS | T | 64 | 13.963 | 85.786 | 203.851 | 1.00 | 38.61 | C |
| ATOM | 6801 | O | CYS | T | 64 | 14.037 | 86.764 | 203.104 | 1.00 | 37.49 | O |
| ATOM | 6803 | N | ARG | T | 65 | 13.423 | 85.845 | 205.066 | 1.00 | 38.27 | N |
| ATOM | 6804 | CA | ARG | T | 65 | 12.914 | 87.105 | 205.613 | 1.00 | 41.06 | C |
| ATOM | 6806 | CB | ARG | T | 65 | 11.383 | 87.131 | 205.585 | 1.00 | 47.00 | C |
| ATOM | 6809 | CG | ARG | T | 65 | 10.782 | 87.082 | 204.179 | 1.00 | 50.39 | C |
| ATOM | 6812 | CD | ARG | T | 65 | 10.847 | 88.433 | 203.464 | 1.00 | 52.14 | C |
| ATOM | 6815 | NE | ARG | T | 65 | 10.815 | 88.280 | 202.008 | 1.00 | 51.81 | N |
| ATOM | 6817 | CZ | ARG | T | 65 | 10.379 | 89.199 | 201.142 | 1.00 | 54.95 | C |
| ATOM | 6818 | NH1 | ARG | T | 65 | 9.914 | 90.378 | 201.558 | 1.00 | 56.38 | N |
| ATOM | 6821 | NH2 | ARG | T | 65 | 10.403 | 88.935 | 199.836 | 1.00 | 54.16 | N |
| ATOM | 6824 | C | ARG | T | 65 | 13.421 | 87.308 | 207.038 | 1.00 | 38.65 | C |
| ATOM | 6825 | O | ARG | T | 65 | 13.521 | 86.341 | 207.799 | 1.00 | 36.99 | O |
| ATOM | 6827 | N | PRO | T | 66 | 13.752 | 88.565 | 207.402 | 1.00 | 37.95 | N |
| ATOM | 6828 | CA | PRO | T | 66 | 14.164 | 88.870 | 208.776 | 1.00 | 38.35 | C |
| ATOM | 6830 | CB | PRO | T | 66 | 14.303 | 90.396 | 208.775 | 1.00 | 38.69 | C |
| ATOM | 6833 | CG | PRO | T | 66 | 14.516 | 90.766 | 207.363 | 1.00 | 38.44 | C |
| ATOM | 6836 | CD | PRO | T | 66 | 13.765 | 89.766 | 206.547 | 1.00 | 38.44 | C |
| ATOM | 6839 | C | PRO | T | 66 | 13.113 | 88.439 | 209.795 | 1.00 | 36.37 | C |
| ATOM | 6840 | O | PRO | T | 66 | 11.919 | 88.584 | 209.535 | 1.00 | 34.32 | O |
| ATOM | 6841 | N | CYS | T | 67 | 13.555 | 87.909 | 210.933 | 1.00 | 36.18 | N |
| ATOM | 6842 | CA | CYS | T | 67 | 12.636 | 87.463 | 211.975 | 1.00 | 38.61 | C |
| ATOM | 6844 | CB | CYS | T | 67 | 13.386 | 86.780 | 213.127 | 1.00 | 38.78 | C |
| ATOM | 6847 | SG | CYS | T | 67 | 14.382 | 85.320 | 212.691 | 1.00 | 39.72 | S |
| ATOM | 6849 | C | CYS | T | 67 | 11.856 | 88.662 | 212.512 | 1.00 | 39.70 | C |
| ATOM | 6850 | O | CYS | T | 67 | 12.437 | 89.712 | 212.795 | 1.00 | 40.88 | O |
| ATOM | 6852 | N | GLY | T | 68 | 10.541 | 88.504 | 212.640 | 1.00 | 39.56 | N |
| ATOM | 6853 | CA | GLY | T | 68 | 9.679 | 89.577 | 213.125 | 1.00 | 39.84 | C |
| ATOM | 6856 | C | GLY | T | 68 | 9.769 | 89.713 | 214.632 | 1.00 | 38.36 | C |
| ATOM | 6857 | O | GLY | T | 68 | 10.444 | 88.914 | 215.285 | 1.00 | 37.51 | O |
| ATOM | 6859 | N | PRO | T | 69 | 9.096 | 90.733 | 215.199 | 1.00 | 40.96 | N |
| ATOM | 6860 | CA | PRO | T | 69 | 9.055 | 90.901 | 216.655 | 1.00 | 38.07 | C |
| ATOM | 6862 | CB | PRO | T | 69 | 8.104 | 92.090 | 216.847 | 1.00 | 40.57 | C |
| ATOM | 6865 | CG | PRO | T | 69 | 8.163 | 92.837 | 215.564 | 1.00 | 40.73 | C |
| ATOM | 6868 | CD | PRO | T | 69 | 8.353 | 91.801 | 214.503 | 1.00 | 41.19 | C |
| ATOM | 6871 | C | PRO | T | 69 | 8.527 | 89.659 | 217.378 | 1.00 | 35.48 | C |
| ATOM | 6872 | O | PRO | T | 69 | 7.500 | 89.103 | 216.989 | 1.00 | 35.12 | O |
| ATOM | 6873 | N | GLY | T | 70 | 9.245 | 89.221 | 218.408 | 1.00 | 34.06 | N |
| ATOM | 6874 | CA | GLY | T | 70 | 8.866 | 88.033 | 219.171 | 1.00 | 34.32 | C |
| ATOM | 6877 | C | GLY | T | 70 | 9.373 | 86.721 | 218.588 | 1.00 | 33.68 | C |
| ATOM | 6878 | O | GLY | T | 70 | 9.072 | 85.658 | 219.130 | 1.00 | 29.88 | O |
| ATOM | 6880 | N | PHE | T | 71 | 10.150 | 86.795 | 217.503 | 1.00 | 33.14 | N |
| ATOM | 6881 | CA | PHE | T | 71 | 10.754 | 85.613 | 216.873 | 1.00 | 34.86 | C |
| ATOM | 6883 | CB | PHE | T | 71 | 10.089 | 85.318 | 215.523 | 1.00 | 37.04 | C |
| ATOM | 6886 | CG | PHE | T | 71 | 8.663 | 84.869 | 215.632 | 1.00 | 37.24 | C |
| ATOM | 6887 | CD1 | PHE | T | 71 | 7.637 | 85.795 | 215.789 | 1.00 | 38.30 | C |
| ATOM | 6889 | CE1 | PHE | T | 71 | 6.310 | 85.381 | 215.890 | 1.00 | 38.42 | C |
| ATOM | 6891 | CZ | PHE | T | 71 | 6.002 | 84.028 | 215.830 | 1.00 | 38.39 | C |
| ATOM | 6893 | CE2 | PHE | T | 71 | 7.019 | 83.094 | 215.670 | 1.00 | 37.90 | C |
| ATOM | 6895 | CD2 | PHE | T | 71 | 8.340 | 83.517 | 215.571 | 1.00 | 38.44 | C |
| ATOM | 6897 | C | PHE | T | 71 | 12.254 | 85.804 | 216.651 | 1.00 | 35.17 | C |
| ATOM | 6898 | O | PHE | T | 71 | 12.736 | 86.933 | 216.545 | 1.00 | 34.79 | O |
| ATOM | 6900 | N | TYR | T | 72 | 12.978 | 84.690 | 216.553 | 1.00 | 34.44 | N |
| ATOM | 6901 | CA | TYR | T | 72 | 14.427 | 84.714 | 216.366 | 1.00 | 35.22 | C |
| ATOM | 6903 | CB | TYR | T | 72 | 15.129 | 84.774 | 217.726 | 1.00 | 35.59 | C |
| ATOM | 6906 | CG | TYR | T | 72 | 15.234 | 83.421 | 218.386 | 1.00 | 35.73 | C |
| ATOM | 6907 | CD1 | TYR | T | 72 | 14.143 | 82.864 | 219.050 | 1.00 | 34.26 | C |
| ATOM | 6909 | CE1 | TYR | T | 72 | 14.226 | 81.614 | 219.643 | 1.00 | 35.51 | C |
| ATOM | 6911 | CZ | TYR | T | 72 | 15.406 | 80.898 | 219.566 | 1.00 | 35.51 | C |
| ATOM | 6912 | OH | TYR | T | 72 | 15.493 | 79.660 | 220.157 | 1.00 | 35.96 | O |
| ATOM | 6914 | CE2 | TYR | T | 72 | 16.503 | 81.425 | 218.908 | 1.00 | 36.24 | C |
| ATOM | 6916 | CD2 | TYR | T | 72 | 16.412 | 82.681 | 218.320 | 1.00 | 35.96 | C |
| ATOM | 6918 | C | TYR | T | 72 | 14.938 | 83.492 | 215.601 | 1.00 | 36.85 | C |
| ATOM | 6919 | O | TYR | T | 72 | 14.232 | 82.490 | 215.437 | 1.00 | 34.10 | O |
| ATOM | 6921 | N | ASN | T | 73 | 16.181 | 83.588 | 215.143 | 1.00 | 38.45 | N |
| ATOM | 6922 | CA | ASN | T | 73 | 16.898 | 82.443 | 214.597 | 1.00 | 37.38 | C |
| ATOM | 6924 | CB | ASN | T | 73 | 16.677 | 82.331 | 213.086 | 1.00 | 39.96 | C |
| ATOM | 6927 | CG | ASN | T | 73 | 17.454 | 81.181 | 212.470 | 1.00 | 41.43 | C |
| ATOM | 6928 | OD1 | ASN | T | 73 | 17.481 | 80.074 | 213.015 | 1.00 | 43.13 | O |
| ATOM | 6929 | ND2 | ASN | T | 73 | 18.096 | 81.438 | 211.331 | 1.00 | 42.47 | N |
| ATOM | 6932 | C | ASN | T | 73 | 18.388 | 82.569 | 214.914 | 1.00 | 36.67 | C |
| ATOM | 6933 | O | ASN | T | 73 | 19.042 | 83.500 | 214.453 | 1.00 | 34.57 | O |
| ATOM | 6935 | N | ASP | T | 74 | 18.909 | 81.625 | 215.698 | 1.00 | 35.89 | N |
| ATOM | 6936 | CA | ASP | T | 74 | 20.299 | 81.671 | 216.162 | 1.00 | 36.94 | C |
| ATOM | 6938 | CB | ASP | T | 74 | 20.383 | 81.257 | 217.643 | 1.00 | 35.94 | C |
| ATOM | 6941 | CG | ASP | T | 74 | 19.951 | 79.818 | 217.890 | 1.00 | 36.88 | C |
| ATOM | 6942 | OD1 | ASP | T | 74 | 19.087 | 79.299 | 217.150 | 1.00 | 37.53 | O |
| ATOM | 6943 | OD2 | ASP | T | 74 | 20.465 | 79.208 | 218.851 | 1.00 | 40.14 | O |
| ATOM | 6944 | C | ASP | T | 74 | 21.281 | 80.845 | 215.314 | 1.00 | 36.41 | C |
| ATOM | 6945 | O | ASP | T | 74 | 22.488 | 80.875 | 215.564 | 1.00 | 34.75 | O |
| ATOM | 6947 | N | VAL | T | 75 | 20.773 | 80.135 | 214.309 | 1.00 | 35.84 | N |
| ATOM | 6948 | CA | VAL | T | 75 | 21.609 | 79.282 | 213.455 | 1.00 | 36.04 | C |
| ATOM | 6950 | CB | VAL | T | 75 | 21.427 | 77.777 | 213.796 | 1.00 | 35.40 | C |
| ATOM | 6952 | CG1 | VAL | T | 75 | 21.832 | 77.502 | 215.238 | 1.00 | 36.11 | C |
| ATOM | 6956 | CG2 | VAL | T | 75 | 19.987 | 77.318 | 213.539 | 1.00 | 34.73 | C |
| ATOM | 6960 | C | VAL | T | 75 | 21.303 | 79.487 | 211.974 | 1.00 | 37.17 | C |
| ATOM | 6961 | O | VAL | T | 75 | 20.328 | 80.149 | 211.615 | 1.00 | 39.09 | O |
| ATOM | 6963 | N | VAL | T | 76 | 22.147 | 78.917 | 211.117 | 1.00 | 37.02 | N |
| ATOM | 6964 | CA | VAL | T | 76 | 21.871 | 78.866 | 209.685 | 1.00 | 36.47 | C |
| ATOM | 6966 | CB | VAL | T | 76 | 23.127 | 78.470 | 208.878 | 1.00 | 37.75 | C |
| ATOM | 6968 | CG1 | VAL | T | 76 | 22.768 | 78.137 | 207.428 | 1.00 | 38.65 | C |
| ATOM | 6972 | CG2 | VAL | T | 76 | 24.165 | 79.583 | 208.938 | 1.00 | 38.43 | C |
| ATOM | 6976 | C | VAL | T | 76 | 20.788 | 77.817 | 209.479 | 1.00 | 35.92 | C |
| ATOM | 6977 | O | VAL | T | 76 | 20.914 | 76.704 | 209.986 | 1.00 | 34.52 | O |
| ATOM | 6979 | N | SER | T | 77 | 19.728 | 78.160 | 208.748 | 1.00 | 36.66 | N |
| ATOM | 6980 | CA | SER | T | 77 | 18.611 | 77.231 | 208.587 | 1.00 | 35.83 | C |
| ATOM | 6982 | CB | SER | T | 77 | 17.664 | 77.332 | 209.788 | 1.00 | 38.75 | C |
| ATOM | 6985 | OG | SER | T | 77 | 16.791 | 78.441 | 209.654 | 1.00 | 40.91 | O |
| ATOM | 6987 | C | SER | T | 77 | 17.799 | 77.404 | 207.304 | 1.00 | 33.75 | C |
| ATOM | 6988 | O | SER | T | 77 | 17.921 | 78.394 | 206.578 | 1.00 | 32.41 | O |
| ATOM | 6990 | N | SER | T | 78 | 16.969 | 76.402 | 207.052 | 1.00 | 33.95 | N |
| ATOM | 6991 | CA | SER | T | 78 | 15.994 | 76.420 | 205.972 | 1.00 | 33.65 | C |
| ATOM | 6993 | CB | SER | T | 78 | 16.284 | 75.281 | 205.002 | 1.00 | 35.05 | C |
| ATOM | 6996 | OG | SER | T | 78 | 17.557 | 75.451 | 204.401 | 1.00 | 38.47 | O |
| ATOM | 6998 | C | SER | T | 78 | 14.598 | 76.285 | 206.575 | 1.00 | 30.99 | C |
| ATOM | 6999 | O | SER | T | 78 | 13.702 | 75.690 | 205.975 | 1.00 | 31.02 | O |
| ATOM | 7001 | N | LYS | T | 79 | 14.422 | 76.857 | 207.763 | 1.00 | 30.65 | N |
| ATOM | 7002 | CA | LYS | T | 79 | 13.141 | 76.833 | 208.466 | 1.00 | 32.43 | C |
| ATOM | 7004 | CB | LYS | T | 79 | 13.249 | 75.956 | 209.721 | 1.00 | 34.16 | C |
| ATOM | 7007 | CG | LYS | T | 79 | 13.583 | 74.496 | 209.419 | 1.00 | 33.57 | C |
| ATOM | 7010 | CD | LYS | T | 79 | 13.189 | 73.550 | 210.559 | 1.00 | 35.21 | C |
| ATOM | 7013 | CE | LYS | T | 79 | 13.813 | 72.173 | 210.376 | 1.00 | 34.50 | C |
| ATOM | 7016 | NZ | LYS | T | 79 | 13.631 | 71.272 | 211.550 | 1.00 | 31.45 | N |
| ATOM | 7020 | C | LYS | T | 79 | 12.717 | 78.255 | 208.849 | 1.00 | 32.96 | C |
| ATOM | 7021 | O | LYS | T | 79 | 13.556 | 79.164 | 208.898 | 1.00 | 31.21 | O |
| ATOM | 7023 | N | PRO | T | 80 | 11.412 | 78.464 | 209.110 | 1.00 | 31.23 | N |
| ATOM | 7024 | CA | PRO | T | 80 | 10.959 | 79.749 | 209.653 | 1.00 | 33.72 | C |
| ATOM | 7026 | CB | PRO | T | 80 | 9.453 | 79.530 | 209.870 | 1.00 | 33.31 | C |
| ATOM | 7029 | CG | PRO | T | 80 | 9.094 | 78.437 | 208.948 | 1.00 | 33.07 | C |
| ATOM | 7032 | CD | PRO | T | 80 | 10.286 | 77.538 | 208.901 | 1.00 | 33.53 | C |
| ATOM | 7035 | C | PRO | T | 80 | 11.631 | 80.083 | 210.981 | 1.00 | 31.68 | C |
| ATOM | 7036 | O | PRO | T | 80 | 12.204 | 79.198 | 211.621 | 1.00 | 28.73 | O |
| ATOM | 7037 | N | CYS | T | 81 | 11.566 | 81.348 | 211.385 | 1.00 | 34.79 | N |
| ATOM | 7038 | CA | CYS | T | 81 | 12.065 | 81.765 | 212.701 | 1.00 | 36.06 | C |
| ATOM | 7040 | CB | CYS | T | 81 | 12.071 | 83.289 | 212.828 | 1.00 | 37.56 | C |
| ATOM | 7043 | SG | CYS | T | 81 | 13.132 | 84.102 | 211.616 | 1.00 | 40.87 | S |
| ATOM | 7045 | C | CYS | T | 81 | 11.217 | 81.147 | 213.811 | 1.00 | 37.25 | C |
| ATOM | 7046 | O | CYS | T | 81 | 10.079 | 80.747 | 213.579 | 1.00 | 37.93 | O |
| ATOM | 7048 | N | LYS | T | 82 | 11.789 | 81.071 | 215.010 | 1.00 | 34.02 | N |
| ATOM | 7049 | CA | LYS | T | 82 | 11.169 | 80.393 | 216.146 | 1.00 | 36.28 | C |
| ATOM | 7051 | CB | LYS | T | 82 | 12.188 | 79.464 | 216.812 | 1.00 | 38.79 | C |
| ATOM | 7054 | CG | LYS | T | 82 | 12.826 | 78.461 | 215.859 | 1.00 | 41.73 | C |
| ATOM | 7057 | CD | LYS | T | 82 | 13.999 | 77.738 | 216.504 | 1.00 | 42.70 | C |
| ATOM | 7060 | CE | LYS | T | 82 | 15.266 | 78.581 | 216.485 | 1.00 | 43.74 | C |
| ATOM | 7063 | NZ | LYS | T | 82 | 16.352 | 77.951 | 217.287 | 1.00 | 44.66 | N |
| ATOM | 7067 | C | LYS | T | 82 | 10.682 | 81.431 | 217.158 | 1.00 | 33.84 | C |
| ATOM | 7068 | O | LYS | T | 82 | 11.263 | 82.509 | 217.250 | 1.00 | 32.50 | O |
| ATOM | 7070 | N | PRO | T | 83 | 9.612 | 81.119 | 217.911 | 1.00 | 33.44 | N |
| ATOM | 7071 | CA | PRO | T | 83 | 9.161 | 82.078 | 218.932 | 1.00 | 34.44 | C |
| ATOM | 7073 | CB | PRO | T | 83 | 7.864 | 81.456 | 219.471 | 1.00 | 35.09 | C |
| ATOM | 7076 | CG | PRO | T | 83 | 7.500 | 80.365 | 218.516 | 1.00 | 35.63 | C |
| ATOM | 7079 | CD | PRO | T | 83 | 8.766 | 79.914 | 217.870 | 1.00 | 34.07 | C |
| ATOM | 7082 | C | PRO | T | 83 | 10.180 | 82.255 | 220.060 | 1.00 | 33.45 | C |
| ATOM | 7083 | O | PRO | T | 83 | 10.873 | 81.302 | 220.424 | 1.00 | 31.63 | O |
| ATOM | 7084 | N | CYS | T | 84 | 10.269 | 83.470 | 220.597 | 1.00 | 34.05 | N |
| ATOM | 7085 | CA | CYS | T | 84 | 11.143 | 83.756 | 221.732 | 1.00 | 35.54 | C |
| ATOM | 7087 | CB | CYS | T | 84 | 11.298 | 85.264 | 221.925 | 1.00 | 37.11 | C |
| ATOM | 7090 | SG | CYS | T | 84 | 11.837 | 86.114 | 220.439 | 1.00 | 41.83 | S |
| ATOM | 7092 | C | CYS | T | 84 | 10.589 | 83.149 | 223.019 | 1.00 | 36.56 | C |
| ATOM | 7093 | O | CYS | T | 84 | 9.370 | 83.028 | 223.187 | 1.00 | 34.52 | O |
| ATOM | 7095 | N | THR | T | 85 | 11.493 | 82.795 | 223.928 | 1.00 | 34.32 | N |
| ATOM | 7096 | CA | THR | T | 85 | 11.122 | 82.188 | 225.199 | 1.00 | 35.41 | C |
| ATOM | 7098 | CB | THR | T | 85 | 12.311 | 81.397 | 225.795 | 1.00 | 35.95 | C |
| ATOM | 7100 | OG1 | THR | T | 85 | 12.806 | 80.474 | 224.816 | 1.00 | 34.26 | O |
| ATOM | 7102 | CG2 | THR | T | 85 | 11.896 | 80.630 | 227.044 | 1.00 | 36.36 | C |
| ATOM | 7106 | C | THR | T | 85 | 10.670 | 83.251 | 226.201 | 1.00 | 34.77 | C |
| ATOM | 7107 | O | THR | T | 85 | 11.204 | 84.364 | 226.215 | 1.00 | 35.28 | O |
| ATOM | 7109 | N | TRP | T | 86 | 9.675 | 82.899 | 227.017 | 1.00 | 34.73 | N |
| ATOM | 7110 | CA | TRP | T | 86 | 9.278 | 83.692 | 228.189 | 1.00 | 35.03 | C |
| ATOM | 7112 | CB | TRP | T | 86 | 7.773 | 83.563 | 228.460 | 1.00 | 39.45 | C |
| ATOM | 7115 | CG | TRP | T | 86 | 6.839 | 83.986 | 227.353 | 1.00 | 39.38 | C |
| ATOM | 7116 | CD1 | TRP | T | 86 | 6.281 | 83.183 | 226.398 | 1.00 | 41.17 | C |
| ATOM | 7118 | NE1 | TRP | T | 86 | 5.459 | 83.916 | 225.576 | 1.00 | 41.33 | N |
| ATOM | 7120 | CE2 | TRP | T | 86 | 5.458 | 85.219 | 226.000 | 1.00 | 40.59 | C |
| ATOM | 7121 | CD2 | TRP | T | 86 | 6.313 | 85.301 | 227.122 | 1.00 | 39.70 | C |
| ATOM | 7122 | CE3 | TRP | T | 86 | 6.486 | 86.541 | 227.750 | 1.00 | 40.74 | C |
| ATOM | 7124 | CZ3 | TRP | T | 86 | 5.810 | 87.648 | 227.242 | 1.00 | 40.64 | C |
| ATOM | 7126 | CH2 | TRP | T | 86 | 4.971 | 87.534 | 226.122 | 1.00 | 40.91 | C |
| ATOM | 7128 | CZ2 | TRP | T | 86 | 4.782 | 86.332 | 225.488 | 1.00 | 41.10 | C |
| ATOM | 7130 | C | TRP | T | 86 | 10.018 | 83.179 | 229.430 | 1.00 | 33.20 | C |
| ATOM | 7131 | O | TRP | T | 86 | 10.175 | 81.969 | 229.611 | 1.00 | 30.39 | O |
| ATOM | 7133 | N | CYS | T | 87 | 10.456 | 84.094 | 230.295 | 1.00 | 31.70 | N |
| ATOM | 7134 | CA | CYS | T | 87 | 11.019 | 83.713 | 231.593 | 1.00 | 32.58 | C |
| ATOM | 7136 | CB | CYS | T | 87 | 11.905 | 84.834 | 232.161 | 1.00 | 33.42 | C |
| ATOM | 7139 | SG | CYS | T | 87 | 13.267 | 85.411 | 231.086 | 1.00 | 34.91 | S |
| ATOM | 7141 | C | CYS | T | 87 | 9.880 | 83.413 | 232.569 | 1.00 | 30.26 | C |
| ATOM | 7142 | O | CYS | T | 87 | 8.871 | 84.121 | 232.582 | 1.00 | 29.56 | O |
| ATOM | 7144 | N | ASN | T | 88 | 10.038 | 82.364 | 233.375 | 1.00 | 30.25 | N |
| ATOM | 7145 | CA | ASN | T | 88 | 9.044 | 82.016 | 234.398 | 1.00 | 32.12 | C |
| ATOM | 7147 | CB | ASN | T | 88 | 9.155 | 80.536 | 234.788 | 1.00 | 30.70 | C |
| ATOM | 7150 | CG | ASN | T | 88 | 7.979 | 80.049 | 235.631 | 1.00 | 31.82 | C |
| ATOM | 7151 | OD1 | ASN | T | 88 | 7.840 | 78.849 | 235.864 | 1.00 | 32.91 | O |
| ATOM | 7152 | ND2 | ASN | T | 88 | 7.134 | 80.972 | 236.093 | 1.00 | 33.44 | N |
| ATOM | 7155 | C | ASN | T | 88 | 9.228 | 82.914 | 235.625 | 1.00 | 31.84 | C |
| ATOM | 7156 | O | ASN | T | 88 | 9.939 | 82.557 | 236.567 | 1.00 | 31.33 | O |
| ATOM | 7158 | N | LEU | T | 89 | 8.562 | 84.065 | 235.606 | 1.00 | 33.56 | N |
| ATOM | 7159 | CA | LEU | T | 89 | 8.757 | 85.106 | 236.622 | 1.00 | 35.59 | C |
| ATOM | 7161 | CB | LEU | T | 89 | 8.017 | 86.386 | 236.211 | 1.00 | 37.38 | C |
| ATOM | 7164 | CG | LEU | T | 89 | 8.442 | 87.020 | 234.880 | 1.00 | 39.15 | C |
| ATOM | 7166 | CD1 | LEU | T | 89 | 7.377 | 87.991 | 234.386 | 1.00 | 40.53 | C |
| ATOM | 7170 | CD2 | LEU | T | 89 | 9.791 | 87.712 | 235.004 | 1.00 | 39.68 | C |
| ATOM | 7174 | C | LEU | T | 89 | 8.317 | 84.683 | 238.030 | 1.00 | 36.14 | C |
| ATOM | 7175 | O | LEU | T | 89 | 8.926 | 85.092 | 239.023 | 1.00 | 35.72 | O |
| ATOM | 7177 | N | ARG | T | 90 | 7.267 | 83.868 | 238.107 | 1.00 | 37.32 | N |
| ATOM | 7178 | CA | ARG | T | 90 | 6.726 | 83.407 | 239.387 | 1.00 | 39.48 | C |
| ATOM | 7180 | CB | ARG | T | 90 | 5.344 | 82.768 | 239.192 | 1.00 | 43.48 | C |
| ATOM | 7183 | CG | ARG | T | 90 | 4.303 | 83.716 | 238.607 | 1.00 | 45.53 | C |
| ATOM | 7186 | CD | ARG | T | 90 | 2.931 | 83.057 | 238.448 | 1.00 | 46.70 | C |
| ATOM | 7189 | NE | ARG | T | 90 | 2.192 | 83.619 | 237.311 | 1.00 | 48.12 | N |
| ATOM | 7191 | CZ | ARG | T | 90 | 0.862 | 83.684 | 237.203 | 1.00 | 49.64 | C |
| ATOM | 7192 | NH1 | ARG | T | 90 | 0.063 | 83.228 | 238.167 | 1.00 | 50.29 | N |
| ATOM | 7195 | NH2 | ARG | T | 90 | 0.323 | 84.224 | 236.113 | 1.00 | 49.28 | N |
| ATOM | 7198 | C | ARG | T | 90 | 7.661 | 82.418 | 240.085 | 1.00 | 37.77 | C |
| ATOM | 7199 | O | ARG | T | 90 | 7.713 | 82.372 | 241.317 | 1.00 | 37.13 | O |
| ATOM | 7201 | N | SER | T | 91 | 8.400 | 81.637 | 239.298 | 1.00 | 35.78 | N |
| ATOM | 7202 | CA | SER | T | 91 | 9.329 | 80.646 | 239.843 | 1.00 | 36.49 | C |
| ATOM | 7204 | CB | SER | T | 91 | 9.668 | 79.594 | 238.785 | 1.00 | 36.19 | C |
| ATOM | 7207 | OG | SER | T | 91 | 10.583 | 80.097 | 237.827 | 1.00 | 36.85 | O |
| ATOM | 7209 | C | SER | T | 91 | 10.622 | 81.271 | 240.378 | 1.00 | 36.78 | C |
| ATOM | 7210 | O | SER | T | 91 | 11.348 | 80.633 | 241.144 | 1.00 | 37.36 | O |
| ATOM | 7212 | N | GLY | T | 92 | 10.904 | 82.510 | 239.973 | 1.00 | 36.33 | N |
| ATOM | 7213 | CA | GLY | T | 92 | 12.111 | 83.222 | 240.400 | 1.00 | 35.60 | C |
| ATOM | 7216 | C | GLY | T | 92 | 13.012 | 83.669 | 239.258 | 1.00 | 33.96 | C |
| ATOM | 7217 | O | GLY | T | 92 | 14.000 | 84.364 | 239.483 | 1.00 | 34.12 | O |
| ATOM | 7219 | N | SER | T | 93 | 12.686 | 83.270 | 238.030 | 1.00 | 35.00 | N |
| ATOM | 7220 | CA | SER | T | 93 | 13.448 | 83.691 | 236.851 | 1.00 | 34.55 | C |
| ATOM | 7222 | CB | SER | T | 93 | 12.994 | 82.894 | 235.623 | 1.00 | 36.29 | C |
| ATOM | 7225 | OG | SER | T | 93 | 13.705 | 83.237 | 234.439 | 1.00 | 33.40 | O |
| ATOM | 7227 | C | SER | T | 93 | 13.272 | 85.192 | 236.607 | 1.00 | 35.84 | C |
| ATOM | 7228 | O | SER | T | 93 | 12.232 | 85.767 | 236.937 | 1.00 | 37.17 | O |
| ATOM | 7230 | N | GLU | T | 94 | 14.304 | 85.819 | 236.052 | 1.00 | 34.32 | N |
| ATOM | 7231 | CA | GLU | T | 94 | 14.264 | 87.236 | 235.703 | 1.00 | 36.25 | C |
| ATOM | 7233 | CB | GLU | T | 94 | 15.067 | 88.071 | 236.710 | 1.00 | 34.77 | C |
| ATOM | 7236 | CG | GLU | T | 94 | 14.536 | 88.034 | 238.144 | 1.00 | 35.35 | C |
| ATOM | 7239 | CD | GLU | T | 94 | 15.382 | 88.855 | 239.117 | 1.00 | 35.70 | C |
| ATOM | 7240 | OE1 | GLU | T | 94 | 16.513 | 89.243 | 238.753 | 1.00 | 37.77 | O |
| ATOM | 7241 | OE2 | GLU | T | 94 | 14.917 | 89.110 | 240.249 | 1.00 | 32.14 | O |
| ATOM | 7242 | C | GLU | T | 94 | 14.855 | 87.406 | 234.312 | 1.00 | 37.24 | C |
| ATOM | 7243 | O | GLU | T | 94 | 15.849 | 86.761 | 233.976 | 1.00 | 37.78 | O |
| ATOM | 7245 | N | ARG | T | 95 | 14.241 | 88.266 | 233.503 | 1.00 | 39.70 | N |
| ATOM | 7246 | CA | ARG | T | 95 | 14.757 | 88.565 | 232.171 | 1.00 | 40.26 | C |
| ATOM | 7248 | CB | ARG | T | 95 | 13.684 | 89.229 | 231.308 | 1.00 | 40.83 | C |
| ATOM | 7251 | CG | ARG | T | 95 | 14.102 | 89.421 | 229.853 | 1.00 | 39.88 | C |
| ATOM | 7254 | CD | ARG | T | 95 | 12.904 | 89.670 | 228.955 | 1.00 | 40.88 | C |
| ATOM | 7257 | NE | ARG | T | 95 | 12.235 | 90.934 | 229.254 | 1.00 | 40.49 | N |
| ATOM | 7259 | CZ | ARG | T | 95 | 11.127 | 91.364 | 228.653 | 1.00 | 41.90 | C |
| ATOM | 7260 | NH1 | ARG | T | 95 | 10.542 | 90.638 | 227.703 | 1.00 | 43.10 | N |
| ATOM | 7263 | NH2 | ARG | T | 95 | 10.601 | 92.535 | 229.000 | 1.00 | 42.48 | N |
| ATOM | 7266 | C | ARG | T | 95 | 15.980 | 89.473 | 232.274 | 1.00 | 41.85 | C |
| ATOM | 7267 | O | ARG | T | 95 | 15.905 | 90.563 | 232.846 | 1.00 | 43.55 | O |
| ATOM | 7269 | N | LYS | T | 96 | 17.101 | 89.004 | 231.732 | 1.00 | 42.69 | N |
| ATOM | 7270 | CA | LYS | T | 96 | 18.345 | 89.772 | 231.697 | 1.00 | 43.17 | C |
| ATOM | 7272 | CB | LYS | T | 96 | 19.559 | 88.835 | 231.808 | 1.00 | 44.18 | C |
| ATOM | 7275 | CG | LYS | T | 96 | 20.896 | 89.474 | 231.415 | 1.00 | 44.19 | C |
| ATOM | 7278 | CD | LYS | T | 96 | 22.089 | 88.669 | 231.926 | 1.00 | 44.45 | C |
| ATOM | 7281 | CE | LYS | T | 96 | 23.370 | 89.498 | 231.917 | 1.00 | 45.12 | C |
| ATOM | 7284 | NZ | LYS | T | 96 | 24.388 | 88.985 | 232.881 | 1.00 | 43.79 | N |
| ATOM | 7288 | C | LYS | T | 96 | 18.419 | 90.567 | 230.401 | 1.00 | 42.89 | C |
| ATOM | 7289 | O | LYS | T | 96 | 18.561 | 91.792 | 230.416 | 1.00 | 40.79 | O |
| ATOM | 7291 | N | GLN | T | 97 | 18.322 | 89.850 | 229.284 | 1.00 | 43.76 | N |
| ATOM | 7292 | CA | GLN | T | 97 | 18.467 | 90.440 | 227.959 | 1.00 | 44.97 | C |
| ATOM | 7294 | CB | GLN | T | 97 | 19.736 | 89.909 | 227.280 | 1.00 | 46.31 | C |
| ATOM | 7297 | CG | GLN | T | 97 | 20.527 | 90.976 | 226.524 | 1.00 | 47.95 | C |
| ATOM | 7300 | CD | GLN | T | 97 | 21.634 | 90.401 | 225.648 | 1.00 | 48.46 | C |
| ATOM | 7301 | OE1 | GLN | T | 97 | 21.573 | 89.249 | 225.212 | 1.00 | 49.32 | O |
| ATOM | 7302 | NE2 | GLN | T | 97 | 22.650 | 91.215 | 225.380 | 1.00 | 50.03 | N |
| ATOM | 7305 | C | GLN | T | 97 | 17.244 | 90.110 | 227.111 | 1.00 | 45.40 | C |
| ATOM | 7306 | O | GLN | T | 97 | 16.793 | 88.961 | 227.077 | 1.00 | 43.17 | O |
| ATOM | 7308 | N | LEU | T | 98 | 16.712 | 91.122 | 226.430 | 1.00 | 45.18 | N |
| ATOM | 7309 | CA | LEU | T | 98 | 15.594 | 90.926 | 225.514 | 1.00 | 44.04 | C |
| ATOM | 7311 | CB | LEU | T | 98 | 15.128 | 92.264 | 224.933 | 1.00 | 44.92 | C |
| ATOM | 7314 | CG | LEU | T | 98 | 14.505 | 93.274 | 225.895 | 1.00 | 46.57 | C |
| ATOM | 7316 | CD1 | LEU | T | 98 | 14.330 | 94.618 | 225.203 | 1.00 | 46.23 | C |
| ATOM | 7320 | CD2 | LEU | T | 98 | 13.177 | 92.759 | 226.424 | 1.00 | 47.22 | C |
| ATOM | 7324 | C | LEU | T | 98 | 15.995 | 90.001 | 224.370 | 1.00 | 43.11 | C |
| ATOM | 7325 | O | LEU | T | 98 | 17.174 | 89.903 | 224.011 | 1.00 | 40.49 | O |
| ATOM | 7327 | N | CYS | T | 99 | 15.010 | 89.313 | 223.804 | 1.00 | 42.59 | N |
| ATOM | 7328 | CA | CYS | T | 99 | 15.243 | 88.521 | 222.607 | 1.00 | 42.84 | C |
| ATOM | 7330 | CB | CYS | T | 99 | 14.052 | 87.617 | 222.314 | 1.00 | 42.83 | C |
| ATOM | 7333 | SG | CYS | T | 99 | 14.357 | 86.459 | 220.973 | 1.00 | 45.54 | S |
| ATOM | 7335 | C | CYS | T | 99 | 15.485 | 89.450 | 221.422 | 1.00 | 40.92 | C |
| ATOM | 7336 | O | CYS | T | 99 | 14.837 | 90.495 | 221.299 | 1.00 | 39.42 | O |
| ATOM | 7338 | N | THR | T | 100 | 16.433 | 89.067 | 220.572 | 1.00 | 41.49 | N |
| ATOM | 7339 | CA | THR | T | 100 | 16.690 | 89.758 | 219.305 | 1.00 | 41.88 | C |
| ATOM | 7341 | CB | THR | T | 100 | 18.131 | 90.328 | 219.226 | 1.00 | 43.92 | C |
| ATOM | 7343 | OG1 | THR | T | 100 | 18.332 | 90.961 | 217.952 | 1.00 | 46.89 | O |
| ATOM | 7345 | CG2 | THR | T | 100 | 19.190 | 89.238 | 219.428 | 1.00 | 42.33 | C |
| ATOM | 7349 | C | THR | T | 100 | 16.442 | 88.774 | 218.166 | 1.00 | 41.73 | C |
| ATOM | 7350 | O | THR | T | 100 | 16.012 | 87.650 | 218.405 | 1.00 | 40.39 | O |
| ATOM | 7352 | N | ALA | T | 101 | 16.710 | 89.201 | 216.935 | 1.00 | 41.45 | N |
| ATOM | 7353 | CA | ALA | T | 101 | 16.556 | 88.343 | 215.763 | 1.00 | 40.33 | C |
| ATOM | 7355 | CB | ALA | T | 101 | 16.733 | 89.160 | 214.486 | 1.00 | 41.27 | C |
| ATOM | 7359 | C | ALA | T | 101 | 17.527 | 87.157 | 215.775 | 1.00 | 39.69 | C |
| ATOM | 7360 | O | ALA | T | 101 | 17.175 | 86.064 | 215.338 | 1.00 | 38.12 | O |
| ATOM | 7362 | N | THR | T | 102 | 18.741 | 87.369 | 216.282 | 1.00 | 37.89 | N |
| ATOM | 7363 | CA | THR | T | 102 | 19.786 | 86.338 | 216.258 | 1.00 | 38.56 | C |
| ATOM | 7365 | CB | THR | T | 102 | 21.165 | 86.960 | 215.946 | 1.00 | 37.78 | C |
| ATOM | 7367 | OG1 | THR | T | 102 | 21.515 | 87.903 | 216.970 | 1.00 | 37.14 | O |
| ATOM | 7369 | CG2 | THR | T | 102 | 21.146 | 87.660 | 214.595 | 1.00 | 36.76 | C |
| ATOM | 7373 | C | THR | T | 102 | 19.915 | 85.530 | 217.557 | 1.00 | 38.71 | C |
| ATOM | 7374 | O | THR | T | 102 | 20.687 | 84.573 | 217.620 | 1.00 | 39.91 | O |
| ATOM | 7376 | N | GLN | T | 103 | 19.156 | 85.893 | 218.585 | 1.00 | 39.28 | N |
| ATOM | 7377 | CA | GLN | T | 103 | 19.401 | 85.365 | 219.923 | 1.00 | 39.83 | C |
| ATOM | 7379 | CB | GLN | T | 103 | 20.416 | 86.261 | 220.652 | 1.00 | 41.15 | C |
| ATOM | 7382 | CG | GLN | T | 103 | 21.305 | 85.530 | 221.649 | 1.00 | 42.91 | C |
| ATOM | 7385 | CD | GLN | T | 103 | 22.159 | 86.475 | 222.485 | 1.00 | 43.06 | C |
| ATOM | 7386 | OE1 | GLN | T | 103 | 22.584 | 87.535 | 222.014 | 1.00 | 44.46 | O |
| ATOM | 7387 | NE2 | GLN | T | 103 | 22.417 | 86.092 | 223.730 | 1.00 | 43.42 | N |
| ATOM | 7390 | C | GLN | T | 103 | 18.105 | 85.287 | 220.717 | 1.00 | 38.58 | C |
| ATOM | 7391 | O | GLN | T | 103 | 17.311 | 86.229 | 220.710 | 1.00 | 37.40 | O |
| ATOM | 7393 | N | ASP | T | 104 | 17.898 | 84.164 | 221.402 | 1.00 | 37.64 | N |
| ATOM | 7394 | CA | ASP | T | 104 | 16.727 | 83.989 | 222.260 | 1.00 | 38.66 | C |
| ATOM | 7396 | CB | ASP | T | 104 | 16.528 | 82.504 | 222.606 | 1.00 | 38.21 | C |
| ATOM | 7399 | CG | ASP | T | 104 | 15.114 | 82.190 | 223.089 | 1.00 | 37.12 | C |
| ATOM | 7400 | OD1 | ASP | T | 104 | 14.228 | 83.069 | 223.005 | 1.00 | 33.93 | O |
| ATOM | 7401 | OD2 | ASP | T | 104 | 14.891 | 81.055 | 223.561 | 1.00 | 38.27 | O |
| ATOM | 7402 | C | ASP | T | 104 | 16.856 | 84.814 | 223.546 | 1.00 | 38.35 | C |
| ATOM | 7403 | O | ASP | T | 104 | 17.953 | 85.247 | 223.917 | 1.00 | 35.65 | O |
| ATOM | 7405 | N | THR | T | 105 | 15.719 | 85.032 | 224.207 | 1.00 | 38.28 | N |
| ATOM | 7406 | CA | THR | T | 105 | 15.670 | 85.664 | 225.520 | 1.00 | 39.30 | C |
| ATOM | 7408 | CB | THR | T | 105 | 14.264 | 85.516 | 226.155 | 1.00 | 37.98 | C |
| ATOM | 7410 | OG1 | THR | T | 105 | 13.255 | 85.866 | 225.199 | 1.00 | 34.62 | O |
| ATOM | 7412 | CG2 | THR | T | 105 | 14.127 | 86.400 | 227.393 | 1.00 | 37.85 | C |
| ATOM | 7416 | C | THR | T | 105 | 16.681 | 85.030 | 226.472 | 1.00 | 40.61 | C |
| ATOM | 7417 | O | THR | T | 105 | 16.863 | 83.812 | 226.468 | 1.00 | 40.51 | O |
| ATOM | 7419 | N | VAL | T | 106 | 17.340 | 85.867 | 227.272 | 1.00 | 40.18 | N |
| ATOM | 7420 | CA | VAL | T | 106 | 18.242 | 85.400 | 228.323 | 1.00 | 41.65 | C |
| ATOM | 7422 | CB | VAL | T | 106 | 19.537 | 86.246 | 228.391 | 1.00 | 42.10 | C |
| ATOM | 7424 | CG1 | VAL | T | 106 | 20.465 | 85.725 | 229.485 | 1.00 | 40.85 | C |
| ATOM | 7428 | CG2 | VAL | T | 106 | 20.245 | 86.240 | 227.036 | 1.00 | 42.75 | C |
| ATOM | 7432 | C | VAL | T | 106 | 17.510 | 85.478 | 229.660 | 1.00 | 40.61 | C |
| ATOM | 7433 | O | VAL | T | 106 | 17.122 | 86.560 | 230.099 | 1.00 | 38.96 | O |
| ATOM | 7435 | N | CYS | T | 107 | 17.304 | 84.319 | 230.282 | 1.00 | 41.34 | N |
| ATOM | 7436 | CA | CYS | T | 107 | 16.630 | 84.226 | 231.569 | 1.00 | 43.71 | C |
| ATOM | 7438 | CB | CYS | T | 107 | 15.432 | 83.277 | 231.490 | 1.00 | 40.41 | C |
| ATOM | 7441 | SG | CYS | T | 107 | 14.120 | 83.728 | 230.314 | 1.00 | 35.76 | S |
| ATOM | 7443 | C | CYS | T | 107 | 17.624 | 83.701 | 232.593 | 1.00 | 46.38 | C |
| ATOM | 7444 | O | CYS | T | 107 | 18.380 | 82.774 | 232.308 | 1.00 | 45.77 | O |
| ATOM | 7446 | N | ARG | T | 108 | 17.624 | 84.300 | 233.779 | 1.00 | 49.86 | N |
| ATOM | 7447 | CA | ARG | T | 108 | 18.496 | 83.871 | 234.867 | 1.00 | 52.45 | C |
| ATOM | 7449 | CB | ARG | T | 108 | 19.686 | 84.823 | 235.003 | 1.00 | 54.11 | C |
| ATOM | 7452 | CG | ARG | T | 108 | 20.711 | 84.726 | 233.878 | 1.00 | 54.49 | C |
| ATOM | 7455 | CD | ARG | T | 108 | 21.582 | 83.484 | 234.012 | 1.00 | 55.09 | C |
| ATOM | 7458 | NE | ARG | T | 108 | 22.663 | 83.465 | 233.025 | 1.00 | 55.14 | N |
| ATOM | 7460 | CZ | ARG | T | 108 | 22.551 | 83.041 | 231.765 | 1.00 | 55.49 | C |
| ATOM | 7461 | NH1 | ARG | T | 108 | 21.394 | 82.587 | 231.292 | 1.00 | 55.65 | N |
| ATOM | 7464 | NH2 | ARG | T | 108 | 23.610 | 83.074 | 230.964 | 1.00 | 55.40 | N |
| ATOM | 7467 | C | ARG | T | 108 | 17.706 | 83.825 | 236.169 | 1.00 | 53.32 | C |
| ATOM | 7468 | O | ARG | T | 108 | 16.776 | 84.610 | 236.368 | 1.00 | 51.51 | O |
| ATOM | 7470 | N | CYS | T | 109 | 18.078 | 82.897 | 237.047 | 1.00 | 54.58 | N |
| ATOM | 7471 | CA | CYS | T | 109 | 17.429 | 82.756 | 238.349 | 1.00 | 60.22 | C |
| ATOM | 7473 | CB | CYS | T | 109 | 17.622 | 81.335 | 238.891 | 1.00 | 60.35 | C |
| ATOM | 7476 | SG | CYS | T | 109 | 16.918 | 80.011 | 237.819 | 1.00 | 64.74 | S |
| ATOM | 7478 | C | CYS | T | 109 | 17.975 | 83.795 | 239.336 | 1.00 | 61.34 | C |
| ATOM | 7479 | O | CYS | T | 109 | 19.178 | 84.048 | 239.374 | 1.00 | 62.81 | O |
| ATOM | 7481 | N | ARG | T | 110 | 17.071 | 84.388 | 240.117 | 1.00 | 64.12 | N |
| ATOM | 7482 | CA | ARG | T | 110 | 17.420 | 85.399 | 241.121 | 1.00 | 65.20 | C |
| ATOM | 7484 | CB | ARG | T | 110 | 16.161 | 86.135 | 241.608 | 1.00 | 67.88 | C |
| ATOM | 7487 | CG | ARG | T | 110 | 15.180 | 85.275 | 242.419 | 1.00 | 69.84 | C |
| ATOM | 7490 | CD | ARG | T | 110 | 14.065 | 86.103 | 243.053 | 1.00 | 71.53 | C |
| ATOM | 7493 | NE | ARG | T | 110 | 13.181 | 86.700 | 242.052 | 1.00 | 73.16 | N |
| ATOM | 7495 | CZ | ARG | T | 110 | 11.989 | 87.238 | 242.309 | 1.00 | 73.85 | C |
| ATOM | 7496 | NH1 | ARG | T | 110 | 11.495 | 87.258 | 243.544 | 1.00 | 74.50 | N |
| ATOM | 7499 | NH2 | ARG | T | 110 | 11.275 | 87.755 | 241.313 | 1.00 | 73.76 | N |
| ATOM | 7502 | C | ARG | T | 110 | 18.134 | 84.770 | 242.310 | 1.00 | 64.78 | C |
| ATOM | 7503 | O | ARG | T | 110 | 18.246 | 83.543 | 242.403 | 1.00 | 65.69 | O |
| ATOM | 7505 | N | ALA | T | 111 | 18.608 | 85.621 | 243.218 | 1.00 | 62.71 | N |
| ATOM | 7506 | CA | ALA | T | 111 | 19.181 | 85.153 | 244.467 | 1.00 | 62.56 | C |
| ATOM | 7508 | CB | ALA | T | 111 | 19.792 | 86.321 | 245.257 | 1.00 | 63.12 | C |
| ATOM | 7512 | C | ALA | T | 111 | 18.078 | 84.458 | 245.264 | 1.00 | 59.72 | C |
| ATOM | 7513 | O | ALA | T | 111 | 16.909 | 84.867 | 245.211 | 1.00 | 57.89 | O |
| ATOM | 7515 | N | GLY | T | 112 | 18.445 | 83.377 | 245.953 | 1.00 | 58.06 | N |
| ATOM | 7516 | CA | GLY | T | 112 | 17.490 | 82.596 | 246.741 | 1.00 | 58.62 | C |
| ATOM | 7519 | C | GLY | T | 112 | 16.776 | 81.503 | 245.963 | 1.00 | 56.88 | C |
| ATOM | 7520 | O | GLY | T | 112 | 15.934 | 80.793 | 246.517 | 1.00 | 52.57 | O |
| ATOM | 7522 | N | THR | T | 113 | 17.102 | 81.366 | 244.679 | 1.00 | 56.21 | N |
| ATOM | 7523 | CA | THR | T | 113 | 16.545 | 80.298 | 243.848 | 1.00 | 57.52 | C |
| ATOM | 7525 | CB | THR | T | 113 | 15.354 | 80.792 | 242.979 | 1.00 | 54.77 | C |
| ATOM | 7527 | OG1 | THR | T | 113 | 15.844 | 81.558 | 241.871 | 1.00 | 49.72 | O |
| ATOM | 7529 | CG2 | THR | T | 113 | 14.384 | 81.651 | 243.798 | 1.00 | 54.74 | C |
| ATOM | 7533 | C | THR | T | 113 | 17.627 | 79.689 | 242.948 | 1.00 | 57.81 | C |
| ATOM | 7534 | O | THR | T | 113 | 18.711 | 80.255 | 242.783 | 1.00 | 57.08 | O |
| ATOM | 7536 | N | GLN | T | 114 | 17.310 | 78.534 | 242.367 | 1.00 | 58.56 | N |
| ATOM | 7537 | CA | GLN | T | 114 | 18.266 | 77.741 | 241.596 | 1.00 | 58.71 | C |
| ATOM | 7539 | CB | GLN | T | 114 | 18.820 | 76.634 | 242.493 | 1.00 | 59.14 | C |
| ATOM | 7542 | CG | GLN | T | 114 | 19.925 | 75.787 | 241.881 | 1.00 | 59.82 | C |
| ATOM | 7545 | CD | GLN | T | 114 | 20.364 | 74.649 | 242.791 | 1.00 | 59.58 | C |
| ATOM | 7546 | OE1 | GLN | T | 114 | 19.730 | 74.366 | 243.808 | 1.00 | 57.96 | O |
| ATOM | 7547 | NE2 | GLN | T | 114 | 21.452 | 73.985 | 242.420 | 1.00 | 61.17 | N |
| ATOM | 7550 | C | GLN | T | 114 | 17.573 | 77.123 | 240.367 | 1.00 | 57.16 | C |
| ATOM | 7551 | O | GLN | T | 114 | 16.429 | 76.676 | 240.470 | 1.00 | 51.14 | O |
| ATOM | 7553 | N | PRO | T | 115 | 18.262 | 77.086 | 239.205 | 1.00 | 58.01 | N |
| ATOM | 7554 | CA | PRO | T | 115 | 17.615 | 76.551 | 237.992 | 1.00 | 61.65 | C |
| ATOM | 7556 | CB | PRO | T | 115 | 18.651 | 76.802 | 236.885 | 1.00 | 60.59 | C |
| ATOM | 7559 | CG | PRO | T | 115 | 19.653 | 77.728 | 237.458 | 1.00 | 60.48 | C |
| ATOM | 7562 | CD | PRO | T | 115 | 19.643 | 77.524 | 238.935 | 1.00 | 59.21 | C |
| ATOM | 7565 | C | PRO | T | 115 | 17.283 | 75.058 | 238.061 | 1.00 | 61.12 | C |
| ATOM | 7566 | O | PRO | T | 115 | 17.877 | 74.323 | 238.851 | 1.00 | 58.11 | O |
| ATOM | 7567 | N | LEU | T | 116 | 16.345 | 74.625 | 237.222 | 1.00 | 66.00 | N |
| ATOM | 7568 | CA | LEU | T | 116 | 15.934 | 73.222 | 237.168 | 1.00 | 68.42 | C |
| ATOM | 7570 | CB | LEU | T | 116 | 14.414 | 73.112 | 236.985 | 1.00 | 69.89 | C |
| ATOM | 7573 | CG | LEU | T | 116 | 13.526 | 73.548 | 238.154 | 1.00 | 70.59 | C |
| ATOM | 7575 | CD1 | LEU | T | 116 | 12.062 | 73.497 | 237.746 | 1.00 | 70.65 | C |
| ATOM | 7579 | CD2 | LEU | T | 116 | 13.769 | 72.684 | 239.385 | 1.00 | 71.19 | C |
| ATOM | 7583 | C | LEU | T | 116 | 16.643 | 72.463 | 236.046 | 1.00 | 70.72 | C |
| ATOM | 7584 | O | LEU | T | 116 | 17.484 | 71.601 | 236.306 | 1.00 | 71.34 | O |
| ATOM | 7586 | N | ASP | T | 117 | 16.307 | 72.801 | 234.803 | 1.00 | 72.90 | N |
| ATOM | 7587 | CA | ASP | T | 117 | 16.713 | 72.013 | 233.633 | 1.00 | 72.94 | C |
| ATOM | 7589 | CB | ASP | T | 117 | 15.759 | 72.286 | 232.464 | 1.00 | 73.78 | C |
| ATOM | 7592 | CG | ASP | T | 117 | 14.332 | 71.850 | 232.758 | 1.00 | 74.03 | C |
| ATOM | 7593 | OD1 | ASP | T | 117 | 13.958 | 71.773 | 233.948 | 1.00 | 72.64 | O |
| ATOM | 7594 | OD2 | ASP | T | 117 | 13.582 | 71.586 | 231.792 | 1.00 | 74.25 | O |
| ATOM | 7595 | C | ASP | T | 117 | 18.163 | 72.269 | 233.209 | 1.00 | 74.00 | C |
| ATOM | 7596 | O | ASP | T | 117 | 18.838 | 73.139 | 233.765 | 1.00 | 74.71 | O |
| ATOM | 7598 | N | SER | T | 118 | 18.626 | 71.502 | 232.221 | 1.00 | 74.96 | N |
| ATOM | 7599 | CA | SER | T | 118 | 20.026 | 71.523 | 231.783 | 1.00 | 75.09 | C |
| ATOM | 7601 | CB | SER | T | 118 | 20.546 | 70.088 | 231.649 | 1.00 | 76.20 | C |
| ATOM | 7604 | OG | SER | T | 118 | 21.942 | 70.065 | 231.401 | 1.00 | 76.88 | O |
| ATOM | 7606 | C | SER | T | 118 | 20.243 | 72.268 | 230.462 | 1.00 | 75.03 | C |
| ATOM | 7607 | O | SER | T | 118 | 21.176 | 73.063 | 230.345 | 1.00 | 75.10 | O |
| ATOM | 7609 | N | TYR | T | 119 | 19.393 | 72.003 | 229.470 | 1.00 | 74.86 | N |
| ATOM | 7610 | CA | TYR | T | 119 | 19.572 | 72.567 | 228.123 | 1.00 | 74.21 | C |
| ATOM | 7612 | CB | TYR | T | 119 | 18.791 | 71.750 | 227.082 | 1.00 | 74.15 | C |
| ATOM | 7615 | CG | TYR | T | 119 | 19.421 | 70.411 | 226.754 | 1.00 | 73.91 | C |
| ATOM | 7616 | CD1 | TYR | T | 119 | 20.400 | 70.304 | 225.769 | 1.00 | 74.16 | C |
| ATOM | 7618 | CE1 | TYR | T | 119 | 20.984 | 69.078 | 225.460 | 1.00 | 74.21 | C |
| ATOM | 7620 | CZ | TYR | T | 119 | 20.589 | 67.940 | 226.142 | 1.00 | 74.17 | C |
| ATOM | 7621 | OH | TYR | T | 119 | 21.164 | 66.726 | 225.837 | 1.00 | 73.91 | O |
| ATOM | 7623 | CE2 | TYR | T | 119 | 19.617 | 68.020 | 227.126 | 1.00 | 74.07 | C |
| ATOM | 7625 | CD2 | TYR | T | 119 | 19.038 | 69.252 | 227.425 | 1.00 | 73.82 | C |
| ATOM | 7627 | C | TYR | T | 119 | 19.199 | 74.053 | 228.016 | 1.00 | 73.95 | C |
| ATOM | 7628 | O | TYR | T | 119 | 19.701 | 74.751 | 227.132 | 1.00 | 73.99 | O |
| ATOM | 7630 | N | LYS | T | 120 | 18.325 | 74.528 | 228.905 | 1.00 | 73.13 | N |
| ATOM | 7631 | CA | LYS | T | 120 | 17.924 | 75.940 | 228.938 | 1.00 | 71.96 | C |
| ATOM | 7633 | CB | LYS | T | 120 | 16.663 | 76.173 | 228.129 | 0.00 | 30.00 | C |
| ATOM | 7636 | CG | LYS | T | 120 | 15.543 | 75.418 | 228.842 | 0.00 | 30.00 | C |
| ATOM | 7639 | CD | LYS | T | 120 | 14.210 | 75.643 | 228.130 | 0.00 | 30.00 | C |
| ATOM | 7642 | CE | LYS | T | 120 | 13.091 | 74.888 | 228.842 | 0.00 | 30.00 | C |
| ATOM | 7645 | NZ | LYS | T | 120 | 11.782 | 75.109 | 228.144 | 0.00 | 30.00 | N |
| ATOM | 7649 | C | LYS | T | 120 | 17.922 | 76.490 | 230.367 | 1.00 | 70.28 | C |
| ATOM | 7650 | O | LYS | T | 120 | 16.863 | 76.813 | 230.910 | 1.00 | 66.83 | O |
| ATOM | 7652 | N | PRO | T | 121 | 19.114 | 76.617 | 230.974 | 1.00 | 70.53 | N |
| ATOM | 7653 | CA | PRO | T | 121 | 19.212 | 76.957 | 232.394 | 1.00 | 71.04 | C |
| ATOM | 7655 | CB | PRO | T | 121 | 20.677 | 76.650 | 232.724 | 1.00 | 71.38 | C |
| ATOM | 7658 | CG | PRO | T | 121 | 21.400 | 76.840 | 231.441 | 1.00 | 71.37 | C |
| ATOM | 7661 | CD | PRO | T | 121 | 20.441 | 76.465 | 230.347 | 1.00 | 70.93 | C |
| ATOM | 7664 | C | PRO | T | 121 | 18.886 | 78.420 | 232.699 | 1.00 | 69.78 | C |
| ATOM | 7665 | O | PRO | T | 121 | 19.455 | 79.321 | 232.079 | 1.00 | 67.67 | O |
| ATOM | 7666 | N | GLY | T | 122 | 17.969 | 78.638 | 233.643 | 1.00 | 70.28 | N |
| ATOM | 7667 | CA | GLY | T | 122 | 17.609 | 79.982 | 234.106 | 1.00 | 70.66 | C |
| ATOM | 7670 | C | GLY | T | 122 | 16.169 | 80.406 | 233.819 | 1.00 | 70.33 | C |
| ATOM | 7671 | O | GLY | T | 122 | 15.729 | 81.465 | 234.253 | 1.00 | 76.91 | O |
| ATOM | 7673 | N | VAL | T | 123 | 15.423 | 79.597 | 233.069 | 1.00 | 73.90 | N |
| ATOM | 7674 | CA | VAL | T | 123 | 14.031 | 79.930 | 232.730 | 1.00 | 70.39 | C |
| ATOM | 7676 | CB | VAL | T | 123 | 13.601 | 79.268 | 231.411 | 1.00 | 75.51 | C |
| ATOM | 7678 | CG1 | VAL | T | 123 | 12.109 | 79.526 | 231.132 | 1.00 | 72.57 | C |
| ATOM | 7682 | CG2 | VAL | T | 123 | 14.469 | 79.761 | 230.266 | 1.00 | 75.53 | C |
| ATOM | 7686 | C | VAL | T | 123 | 13.056 | 79.481 | 233.813 | 1.00 | 70.92 | C |
| ATOM | 7687 | O | VAL | T | 123 | 12.151 | 80.242 | 234.231 | 1.00 | 82.85 | O |
| ATOM | 7689 | N | ASP | T | 124 | 13.207 | 78.201 | 234.225 | 1.00 | 65.64 | N |
| ATOM | 7690 | CA | ASP | T | 124 | 12.446 | 77.637 | 235.339 | 1.00 | 65.06 | C |
| ATOM | 7692 | CB | ASP | T | 124 | 11.883 | 76.263 | 234.969 | 1.00 | 65.30 | C |
| ATOM | 7695 | CG | ASP | T | 124 | 11.045 | 76.295 | 233.709 | 1.00 | 63.98 | C |
| ATOM | 7696 | OD1 | ASP | T | 124 | 10.148 | 77.159 | 233.606 | 1.00 | 61.67 | O |
| ATOM | 7697 | OD2 | ASP | T | 124 | 11.283 | 75.449 | 232.823 | 1.00 | 66.17 | O |
| ATOM | 7698 | C | ASP | T | 124 | 13.366 | 77.492 | 236.545 | 1.00 | 63.63 | C |
| ATOM | 7699 | O | ASP | T | 124 | 14.407 | 76.830 | 236.462 | 1.00 | 63.46 | O |
| ATOM | 7701 | N | CYS | T | 125 | 12.983 | 78.106 | 237.662 | 1.00 | 61.40 | N |
| ATOM | 7702 | CA | CYS | T | 125 | 13.804 | 78.092 | 238.869 | 1.00 | 59.40 | C |
| ATOM | 7704 | CB | CYS | T | 125 | 14.245 | 79.518 | 239.202 | 1.00 | 58.64 | C |
| ATOM | 7707 | SG | CYS | T | 125 | 14.918 | 80.393 | 237.749 | 1.00 | 58.68 | S |
| ATOM | 7709 | C | CYS | T | 125 | 13.060 | 77.452 | 240.040 | 1.00 | 57.27 | C |
| ATOM | 7710 | O | CYS | T | 125 | 11.850 | 77.228 | 239.972 | 1.00 | 53.38 | O |
| ATOM | 7712 | N | ALA | T | 126 | 13.799 | 77.151 | 241.106 | 1.00 | 56.21 | N |
| ATOM | 7713 | CA | ALA | T | 126 | 13.234 | 76.533 | 242.304 | 1.00 | 55.90 | C |
| ATOM | 7715 | CB | ALA | T | 126 | 13.377 | 75.023 | 242.232 | 1.00 | 56.15 | C |
| ATOM | 7719 | C | ALA | T | 126 | 13.935 | 77.077 | 243.553 | 1.00 | 53.90 | C |
| ATOM | 7720 | O | ALA | T | 126 | 15.137 | 77.346 | 243.509 | 1.00 | 49.05 | O |
| ATOM | 7722 | N | PRO | T | 127 | 13.194 | 77.228 | 244.670 | 1.00 | 54.33 | N |
| ATOM | 7723 | CA | PRO | T | 127 | 13.775 | 77.830 | 245.880 | 1.00 | 54.43 | C |
| ATOM | 7725 | CB | PRO | T | 127 | 12.589 | 77.881 | 246.857 | 1.00 | 55.09 | C |
| ATOM | 7728 | CG | PRO | T | 127 | 11.369 | 77.694 | 246.025 | 1.00 | 55.15 | C |
| ATOM | 7731 | CD | PRO | T | 127 | 11.784 | 76.847 | 244.877 | 1.00 | 54.67 | C |
| ATOM | 7734 | C | PRO | T | 127 | 14.921 | 77.021 | 246.496 | 1.00 | 53.26 | C |
| ATOM | 7735 | O | PRO | T | 127 | 14.995 | 75.808 | 246.306 | 1.00 | 52.32 | O |
| ATOM | 7736 | N | CYS | T | 128 | 15.804 | 77.699 | 247.228 | 1.00 | 54.27 | N |
| ATOM | 7737 | CA | CYS | T | 128 | 16.865 | 77.026 | 247.979 | 1.00 | 53.86 | C |
| ATOM | 7739 | CB | CYS | T | 128 | 17.809 | 78.040 | 248.633 | 1.00 | 58.25 | C |
| ATOM | 7742 | SG | CYS | T | 128 | 18.741 | 79.095 | 247.503 | 1.00 | 63.91 | S |
| ATOM | 7744 | C | CYS | T | 128 | 16.247 | 76.162 | 249.077 | 1.00 | 51.31 | C |
| ATOM | 7745 | O | CYS | T | 128 | 15.248 | 76.562 | 249.682 | 1.00 | 48.54 | O |
| ATOM | 7747 | N | PRO | T | 129 | 16.826 | 74.973 | 249.337 | 1.00 | 49.59 | N |
| ATOM | 7748 | CA | PRO | T | 129 | 16.378 | 74.206 | 250.499 | 1.00 | 48.98 | C |
| ATOM | 7750 | CB | PRO | T | 129 | 17.129 | 72.871 | 250.363 | 1.00 | 49.02 | C |
| ATOM | 7753 | CG | PRO | T | 129 | 17.633 | 72.836 | 248.958 | 1.00 | 51.39 | C |
| ATOM | 7756 | CD | PRO | T | 129 | 17.874 | 74.262 | 248.587 | 1.00 | 51.44 | C |
| ATOM | 7759 | C | PRO | T | 129 | 16.756 | 74.910 | 251.808 | 1.00 | 46.65 | C |
| ATOM | 7760 | O | PRO | T | 129 | 17.568 | 75.839 | 251.788 | 1.00 | 43.55 | O |
| ATOM | 7761 | N | PRO | T | 130 | 16.170 | 74.476 | 252.939 | 1.00 | 46.60 | N |
| ATOM | 7762 | CA | PRO | T | 130 | 16.508 | 75.078 | 254.229 | 1.00 | 46.26 | C |
| ATOM | 7764 | CB | PRO | T | 130 | 15.761 | 74.195 | 255.236 | 1.00 | 45.70 | C |
| ATOM | 7767 | CG | PRO | T | 130 | 14.645 | 73.602 | 254.471 | 1.00 | 45.90 | C |
| ATOM | 7770 | CD | PRO | T | 130 | 15.160 | 73.411 | 253.078 | 1.00 | 46.51 | C |
| ATOM | 7773 | C | PRO | T | 130 | 18.011 | 75.060 | 254.512 | 1.00 | 46.44 | C |
| ATOM | 7774 | O | PRO | T | 130 | 18.652 | 74.021 | 254.354 | 1.00 | 44.65 | O |
| ATOM | 7775 | N | GLY | T | 131 | 18.561 | 76.205 | 254.916 | 1.00 | 46.37 | N |
| ATOM | 7776 | CA | GLY | T | 131 | 19.978 | 76.310 | 255.265 | 1.00 | 46.88 | C |
| ATOM | 7779 | C | GLY | T | 131 | 20.933 | 76.459 | 254.095 | 1.00 | 47.32 | C |
| ATOM | 7780 | O | GLY | T | 131 | 22.137 | 76.228 | 254.242 | 1.00 | 46.24 | O |
| ATOM | 7782 | N | HIS | T | 132 | 20.400 | 76.847 | 252.938 | 1.00 | 48.02 | N |
| ATOM | 7783 | CA | HIS | T | 132 | 21.206 | 77.061 | 251.739 | 1.00 | 51.04 | C |
| ATOM | 7785 | CB | HIS | T | 132 | 20.825 | 76.058 | 250.647 | 1.00 | 51.16 | C |
| ATOM | 7788 | CG | HIS | T | 132 | 21.330 | 74.669 | 250.891 | 1.00 | 52.13 | C |
| ATOM | 7789 | ND1 | HIS | T | 132 | 22.572 | 74.413 | 251.425 | 1.00 | 52.59 | N |
| ATOM | 7791 | CE1 | HIS | T | 132 | 22.755 | 73.108 | 251.511 | 1.00 | 51.95 | C |
| ATOM | 7793 | NE2 | HIS | T | 132 | 21.679 | 72.506 | 251.041 | 1.00 | 52.08 | N |
| ATOM | 7795 | CD2 | HIS | T | 132 | 20.777 | 73.460 | 250.639 | 1.00 | 53.04 | C |
| ATOM | 7797 | C | HIS | T | 132 | 21.027 | 78.478 | 251.207 | 1.00 | 53.10 | C |
| ATOM | 7798 | O | HIS | T | 132 | 20.027 | 79.138 | 251.499 | 1.00 | 55.47 | O |
| ATOM | 7800 | N | PHE | T | 133 | 22.004 | 78.937 | 250.427 | 1.00 | 54.32 | N |
| ATOM | 7801 | CA | PHE | T | 133 | 21.909 | 80.223 | 249.738 | 1.00 | 54.73 | C |
| ATOM | 7803 | CB | PHE | T | 133 | 22.795 | 81.273 | 250.410 | 1.00 | 54.22 | C |
| ATOM | 7806 | CG | PHE | T | 133 | 24.262 | 81.140 | 250.090 | 1.00 | 54.14 | C |
| ATOM | 7807 | CD1 | PHE | T | 133 | 25.046 | 80.191 | 250.737 | 1.00 | 54.02 | C |
| ATOM | 7809 | CE1 | PHE | T | 133 | 26.400 | 80.072 | 250.453 | 1.00 | 54.02 | C |
| ATOM | 7811 | CZ | PHE | T | 133 | 26.987 | 80.912 | 249.518 | 1.00 | 54.16 | C |
| ATOM | 7813 | CE2 | PHE | T | 133 | 26.217 | 81.870 | 248.872 | 1.00 | 54.19 | C |
| ATOM | 7815 | CD2 | PHE | T | 133 | 24.863 | 81.981 | 249.161 | 1.00 | 53.70 | C |
| ATOM | 7817 | C | PHE | T | 133 | 22.293 | 80.118 | 248.265 | 1.00 | 55.86 | C |
| ATOM | 7818 | O | PHE | T | 133 | 22.947 | 79.164 | 247.844 | 1.00 | 53.63 | O |
| ATOM | 7820 | N | SER | T | 134 | 21.875 | 81.121 | 247.499 | 1.00 | 57.55 | N |
| ATOM | 7821 | CA | SER | T | 134 | 22.341 | 81.317 | 246.134 | 1.00 | 58.72 | C |
| ATOM | 7823 | CB | SER | T | 134 | 21.440 | 80.578 | 245.147 | 1.00 | 59.31 | C |
| ATOM | 7826 | OG | SER | T | 134 | 21.910 | 80.734 | 243.819 | 1.00 | 59.68 | O |
| ATOM | 7828 | C | SER | T | 134 | 22.340 | 82.813 | 245.821 | 1.00 | 60.73 | C |
| ATOM | 7829 | O | SER | T | 134 | 21.340 | 83.484 | 246.065 | 1.00 | 62.90 | O |
| ATOM | 7831 | N | PRO | T | 135 | 23.461 | 83.347 | 245.298 | 1.00 | 61.16 | N |
| ATOM | 7832 | CA | PRO | T | 135 | 23.454 | 84.736 | 244.826 | 1.00 | 60.99 | C |
| ATOM | 7834 | CB | PRO | T | 135 | 24.934 | 85.024 | 244.525 | 1.00 | 61.31 | C |
| ATOM | 7837 | CG | PRO | T | 135 | 25.702 | 83.909 | 245.148 | 1.00 | 61.14 | C |
| ATOM | 7840 | CD | PRO | T | 135 | 24.790 | 82.732 | 245.142 | 1.00 | 60.96 | C |
| ATOM | 7843 | C | PRO | T | 135 | 22.618 | 84.959 | 243.565 | 1.00 | 62.40 | C |
| ATOM | 7844 | O | PRO | T | 135 | 22.329 | 86.109 | 243.228 | 1.00 | 64.14 | O |
| ATOM | 7845 | N | GLY | T | 136 | 22.246 | 83.874 | 242.881 | 1.00 | 60.60 | N |
| ATOM | 7846 | CA | GLY | T | 136 | 21.503 | 83.945 | 241.629 | 1.00 | 60.94 | C |
| ATOM | 7849 | C | GLY | T | 136 | 22.429 | 83.760 | 240.443 | 1.00 | 60.01 | C |
| ATOM | 7850 | O | GLY | T | 136 | 23.541 | 83.244 | 240.589 | 1.00 | 61.56 | O |
| ATOM | 7852 | N | ASP | T | 137 | 21.968 | 84.195 | 239.273 | 1.00 | 58.33 | N |
| ATOM | 7853 | CA | ASP | T | 137 | 22.708 | 84.051 | 238.016 | 1.00 | 57.86 | C |
| ATOM | 7855 | CB | ASP | T | 137 | 23.980 | 84.913 | 238.041 | 1.00 | 58.76 | C |
| ATOM | 7858 | CG | ASP | T | 137 | 24.520 | 85.214 | 236.649 | 1.00 | 58.58 | C |
| ATOM | 7859 | OD1 | ASP | T | 137 | 23.715 | 85.397 | 235.712 | 1.00 | 59.25 | O |
| ATOM | 7860 | OD2 | ASP | T | 137 | 25.758 | 85.280 | 236.497 | 1.00 | 58.95 | O |
| ATOM | 7861 | C | ASP | T | 137 | 23.043 | 82.582 | 237.722 | 1.00 | 56.32 | C |
| ATOM | 7862 | O | ASP | T | 137 | 24.155 | 82.255 | 237.301 | 1.00 | 56.11 | O |
| ATOM | 7864 | N | ASN | T | 138 | 22.072 | 81.703 | 237.963 | 1.00 | 54.25 | N |
| ATOM | 7865 | CA | ASN | T | 138 | 22.222 | 80.277 | 237.674 | 1.00 | 55.50 | C |
| ATOM | 7867 | CB | ASN | T | 138 | 22.449 | 79.665 | 236.347 | 0.00 | 19.08 | C |
| ATOM | 7870 | CG | ASN | T | 138 | 21.185 | 79.614 | 235.513 | 0.00 | 19.54 | C |
| ATOM | 7871 | OD1 | ASN | T | 138 | 20.104 | 79.980 | 235.967 | 0.00 | 18.47 | O |
| ATOM | 7872 | ND2 | ASN | T | 138 | 21.321 | 79.148 | 234.282 | 0.00 | 17.88 | N |
| ATOM | 7875 | C | ASN | T | 138 | 23.160 | 79.506 | 238.605 | 1.00 | 55.03 | C |
| ATOM | 7876 | O | ASN | T | 138 | 23.496 | 78.353 | 238.327 | 1.00 | 56.92 | O |
| ATOM | 7878 | N | GLN | T | 139 | 23.572 | 80.140 | 239.704 | 1.00 | 54.40 | N |
| ATOM | 7879 | CA | GLN | T | 139 | 24.492 | 79.529 | 240.655 | 1.00 | 52.22 | C |
| ATOM | 7881 | CB | GLN | T | 139 | 25.334 | 80.398 | 241.549 | 0.00 | 22.61 | C |
| ATOM | 7884 | CG | GLN | T | 139 | 26.540 | 80.987 | 240.839 | 0.00 | 24.97 | C |
| ATOM | 7887 | CD | GLN | T | 139 | 27.338 | 81.922 | 241.714 | 0.00 | 29.00 | C |
| ATOM | 7888 | OE1 | GLN | T | 139 | 27.730 | 81.563 | 242.823 | 0.00 | 27.17 | O |
| ATOM | 7889 | NE2 | GLN | T | 139 | 27.584 | 83.125 | 241.217 | 0.00 | 31.84 | N |
| ATOM | 7892 | C | GLN | T | 139 | 23.758 | 78.508 | 241.514 | 1.00 | 51.18 | C |
| ATOM | 7893 | O | GLN | T | 139 | 22.632 | 78.757 | 241.956 | 1.00 | 50.76 | O |
| ATOM | 7895 | N | ALA | T | 140 | 24.397 | 77.361 | 241.737 | 1.00 | 50.20 | N |
| ATOM | 7896 | CA | ALA | T | 140 | 23.818 | 76.292 | 242.550 | 1.00 | 48.41 | C |
| ATOM | 7898 | CB | ALA | T | 140 | 24.588 | 74.989 | 242.344 | 1.00 | 48.95 | C |
| ATOM | 7902 | C | ALA | T | 140 | 23.797 | 76.669 | 244.031 | 1.00 | 45.99 | C |
| ATOM | 7903 | O | ALA | T | 140 | 24.607 | 77.480 | 244.488 | 1.00 | 45.62 | O |
| ATOM | 7905 | N | CYS | T | 141 | 22.866 | 76.071 | 244.771 | 1.00 | 43.97 | N |
| ATOM | 7906 | CA | CYS | T | 141 | 22.720 | 76.325 | 246.199 | 1.00 | 43.13 | C |
| ATOM | 7908 | CB | CYS | T | 141 | 21.355 | 75.840 | 246.689 | 1.00 | 43.11 | C |
| ATOM | 7911 | SG | CYS | T | 141 | 19.951 | 76.627 | 245.880 | 1.00 | 42.95 | S |
| ATOM | 7913 | C | CYS | T | 141 | 23.824 | 75.629 | 246.989 | 1.00 | 41.36 | C |
| ATOM | 7914 | O | CYS | T | 141 | 24.165 | 74.481 | 246.704 | 1.00 | 39.71 | O |
| ATOM | 7916 | N | LYS | T | 142 | 24.369 | 76.331 | 247.982 | 1.00 | 39.74 | N |
| ATOM | 7917 | CA | LYS | T | 142 | 25.455 | 75.812 | 248.814 | 1.00 | 39.27 | C |
| ATOM | 7919 | CB | LYS | T | 142 | 26.747 | 76.582 | 248.535 | 1.00 | 39.48 | C |
| ATOM | 7922 | CG | LYS | T | 142 | 27.247 | 76.458 | 247.107 | 1.00 | 39.49 | C |
| ATOM | 7925 | CD | LYS | T | 142 | 28.626 | 77.081 | 246.933 | 1.00 | 39.47 | C |
| ATOM | 7928 | CE | LYS | T | 142 | 29.273 | 76.624 | 245.629 | 1.00 | 40.05 | C |
| ATOM | 7931 | NZ | LYS | T | 142 | 30.651 | 77.165 | 245.448 | 1.00 | 40.15 | N |
| ATOM | 7935 | C | LYS | T | 142 | 25.103 | 75.940 | 250.297 | 1.00 | 37.92 | C |
| ATOM | 7936 | O | LYS | T | 142 | 24.443 | 76.905 | 250.686 | 1.00 | 35.12 | O |
| ATOM | 7938 | N | PRO | T | 143 | 25.538 | 74.968 | 251.130 | 1.00 | 37.46 | N |
| ATOM | 7939 | CA | PRO | T | 143 | 25.329 | 75.046 | 252.583 | 1.00 | 36.92 | C |
| ATOM | 7941 | CB | PRO | T | 143 | 26.148 | 73.868 | 253.130 | 1.00 | 37.18 | C |
| ATOM | 7944 | CG | PRO | T | 143 | 26.333 | 72.945 | 252.005 | 1.00 | 37.15 | C |
| ATOM | 7947 | CD | PRO | T | 143 | 26.240 | 73.733 | 250.737 | 1.00 | 37.49 | C |
| ATOM | 7950 | C | PRO | T | 143 | 25.837 | 76.354 | 253.178 | 1.00 | 36.09 | C |
| ATOM | 7951 | O | PRO | T | 143 | 26.872 | 76.863 | 252.743 | 1.00 | 36.46 | O |
| ATOM | 7952 | N | TRP | T | 144 | 25.109 | 76.900 | 254.151 | 1.00 | 35.32 | N |
| ATOM | 7953 | CA | TRP | T | 144 | 25.581 | 78.082 | 254.869 | 1.00 | 34.63 | C |
| ATOM | 7955 | CB | TRP | T | 144 | 24.540 | 78.594 | 255.873 | 1.00 | 32.92 | C |
| ATOM | 7958 | CG | TRP | T | 144 | 23.335 | 79.229 | 255.262 | 1.00 | 32.22 | C |
| ATOM | 7959 | CD1 | TRP | T | 144 | 23.246 | 79.818 | 254.033 | 1.00 | 32.25 | C |
| ATOM | 7961 | NE1 | TRP | T | 144 | 21.978 | 80.301 | 253.831 | 1.00 | 32.06 | N |
| ATOM | 7963 | CE2 | TRP | T | 144 | 21.221 | 80.043 | 254.943 | 1.00 | 31.60 | C |
| ATOM | 7964 | CD2 | TRP | T | 144 | 22.047 | 79.371 | 255.869 | 1.00 | 31.87 | C |
| ATOM | 7965 | CE3 | TRP | T | 144 | 21.509 | 78.995 | 257.105 | 1.00 | 32.00 | C |
| ATOM | 7967 | CZ3 | TRP | T | 144 | 20.175 | 79.294 | 257.372 | 1.00 | 32.25 | C |
| ATOM | 7969 | CH2 | TRP | T | 144 | 19.379 | 79.961 | 256.427 | 1.00 | 31.91 | C |
| ATOM | 7971 | CZ2 | TRP | T | 144 | 19.883 | 80.344 | 255.212 | 1.00 | 32.37 | C |
| ATOM | 7973 | C | TRP | T | 144 | 26.861 | 77.752 | 255.620 | 1.00 | 33.69 | C |
| ATOM | 7974 | O | TRP | T | 144 | 26.984 | 76.676 | 256.209 | 1.00 | 31.62 | O |
| ATOM | 7976 | N | THR | T | 145 | 27.813 | 78.679 | 255.584 | 1.00 | 35.15 | N |
| ATOM | 7977 | CA | THR | T | 145 | 29.010 | 78.576 | 256.403 | 1.00 | 36.27 | C |
| ATOM | 7979 | CB | THR | T | 145 | 30.050 | 79.655 | 256.031 | 1.00 | 37.26 | C |
| ATOM | 7981 | OG1 | THR | T | 145 | 30.229 | 79.682 | 254.610 | 1.00 | 38.70 | O |
| ATOM | 7983 | CG2 | THR | T | 145 | 31.391 | 79.373 | 256.703 | 1.00 | 37.30 | C |
| ATOM | 7987 | C | THR | T | 145 | 28.598 | 78.752 | 257.863 | 1.00 | 36.05 | C |
| ATOM | 7988 | O | THR | T | 145 | 28.028 | 79.779 | 258.226 | 1.00 | 35.93 | O |
| ATOM | 7990 | N | ASN | T | 146 | 28.864 | 77.735 | 258.679 | 1.00 | 36.96 | N |
| ATOM | 7991 | CA | ASN | T | 146 | 28.566 | 77.779 | 260.107 | 1.00 | 37.64 | C |
| ATOM | 7993 | CB | ASN | T | 146 | 28.317 | 76.363 | 260.644 | 1.00 | 37.11 | C |
| ATOM | 7996 | CG | ASN | T | 146 | 27.552 | 76.357 | 261.959 | 1.00 | 36.74 | C |
| ATOM | 7997 | OD1 | ASN | T | 146 | 27.968 | 76.976 | 262.937 | 1.00 | 35.84 | O |
| ATOM | 7998 | ND2 | ASN | T | 146 | 26.431 | 75.643 | 261.988 | 1.00 | 36.65 | N |
| ATOM | 8001 | C | ASN | T | 146 | 29.718 | 78.456 | 260.852 | 1.00 | 38.34 | C |
| ATOM | 8002 | O | ASN | T | 146 | 30.793 | 77.874 | 261.019 | 1.00 | 37.86 | O |
| ATOM | 8004 | N | CYS | T | 147 | 29.489 | 79.694 | 261.281 | 1.00 | 39.56 | N |
| ATOM | 8005 | CA | CYS | T | 147 | 30.499 | 80.472 | 262.004 | 1.00 | 40.67 | C |
| ATOM | 8007 | CB | CYS | T | 147 | 30.038 | 81.924 | 262.153 | 1.00 | 40.90 | C |
| ATOM | 8010 | SG | CYS | T | 147 | 29.125 | 82.554 | 260.724 | 1.00 | 41.14 | S |
| ATOM | 8012 | C | CYS | T | 147 | 30.785 | 79.881 | 263.384 | 1.00 | 40.94 | C |
| ATOM | 8013 | O | CYS | T | 147 | 31.906 | 79.976 | 263.889 | 1.00 | 40.28 | O |
| ATOM | 8015 | N | THR | T | 148 | 29.764 | 79.264 | 263.978 | 1.00 | 42.22 | N |
| ATOM | 8016 | CA | THR | T | 148 | 29.858 | 78.687 | 265.316 | 1.00 | 43.09 | C |
| ATOM | 8018 | CB | THR | T | 148 | 28.500 | 78.748 | 266.054 | 1.00 | 42.86 | C |
| ATOM | 8020 | OG1 | THR | T | 148 | 27.573 | 77.832 | 265.460 | 1.00 | 44.06 | O |
| ATOM | 8022 | CG2 | THR | T | 148 | 27.921 | 80.155 | 265.986 | 1.00 | 41.95 | C |
| ATOM | 8026 | C | THR | T | 148 | 30.363 | 77.247 | 265.205 | 1.00 | 44.36 | C |
| ATOM | 8027 | O | THR | T | 148 | 29.627 | 76.283 | 265.431 | 1.00 | 44.99 | O |
| ATOM | 8029 | N | LEU | T | 149 | 31.635 | 77.129 | 264.841 | 1.00 | 44.84 | N |
| ATOM | 8030 | CA | LEU | T | 149 | 32.287 | 75.839 | 264.625 | 1.00 | 44.50 | C |
| ATOM | 8032 | CB | LEU | T | 149 | 31.800 | 75.193 | 263.323 | 1.00 | 44.78 | C |
| ATOM | 8035 | CG | LEU | T | 149 | 31.976 | 73.673 | 263.232 | 1.00 | 44.91 | C |
| ATOM | 8037 | CD1 | LEU | T | 149 | 30.909 | 72.958 | 264.051 | 1.00 | 45.01 | C |
| ATOM | 8041 | CD2 | LEU | T | 149 | 31.934 | 73.206 | 261.784 | 1.00 | 44.87 | C |
| ATOM | 8045 | C | LEU | T | 149 | 33.802 | 76.039 | 264.581 | 1.00 | 44.60 | C |
| ATOM | 8046 | O | LEU | T | 149 | 34.550 | 75.334 | 265.261 | 1.00 | 45.41 | O |
| ATOM | 8048 | N | ALA | T | 150 | 34.240 | 77.005 | 263.773 | 1.00 | 43.99 | N |
| ATOM | 8049 | CA | ALA | T | 150 | 35.644 | 77.404 | 263.711 | 1.00 | 44.19 | C |
| ATOM | 8051 | CB | ALA | T | 150 | 35.858 | 78.391 | 262.572 | 1.00 | 44.09 | C |
| ATOM | 8055 | C | ALA | T | 150 | 36.103 | 78.019 | 265.031 | 1.00 | 43.90 | C |
| ATOM | 8056 | O | ALA | T | 150 | 35.400 | 78.839 | 265.623 | 1.00 | 43.66 | O |
| ATOM | 8058 | N | THR | T | 154 | 29.358 | 85.819 | 264.050 | 1.00 | 26.31 | N |
| ATOM | 8059 | CA | THR | T | 154 | 28.553 | 86.131 | 262.873 | 1.00 | 28.57 | C |
| ATOM | 8061 | CB | THR | T | 154 | 27.227 | 85.324 | 262.872 | 1.00 | 29.03 | C |
| ATOM | 8063 | OG1 | THR | T | 154 | 27.507 | 83.927 | 263.035 | 1.00 | 28.77 | O |
| ATOM | 8065 | CG2 | THR | T | 154 | 26.464 | 85.533 | 261.571 | 1.00 | 28.18 | C |
| ATOM | 8069 | C | THR | T | 154 | 28.242 | 87.629 | 262.785 | 1.00 | 28.99 | C |
| ATOM | 8070 | O | THR | T | 154 | 27.356 | 88.127 | 263.481 | 1.00 | 30.37 | O |
| ATOM | 8072 | N | LEU | T | 155 | 28.979 | 88.339 | 261.932 | 1.00 | 28.93 | N |
| ATOM | 8073 | CA | LEU | T | 155 | 28.745 | 89.765 | 261.697 | 1.00 | 28.73 | C |
| ATOM | 8075 | CB | LEU | T | 155 | 29.808 | 90.503 | 261.060 | 0.00 | 30.00 | C |
| ATOM | 8078 | CG | LEU | T | 155 | 31.053 | 90.502 | 261.946 | 0.00 | 30.00 | C |
| ATOM | 8080 | CD1 | LEU | T | 155 | 32.251 | 91.061 | 261.181 | 0.00 | 30.00 | C |
| ATOM | 8084 | CD2 | LEU | T | 155 | 30.783 | 91.360 | 263.183 | 0.00 | 30.00 | C |
| ATOM | 8088 | C | LEU | T | 155 | 27.414 | 89.993 | 260.979 | 1.00 | 28.71 | C |
| ATOM | 8089 | O | LEU | T | 155 | 26.642 | 90.877 | 261.356 | 1.00 | 27.81 | O |
| ATOM | 8091 | N | GLN | T | 156 | 27.165 | 89.194 | 259.940 | 1.00 | 28.97 | N |
| ATOM | 8092 | CA | GLN | T | 156 | 25.882 | 89.189 | 259.233 | 1.00 | 29.48 | C |
| ATOM | 8094 | CB | GLN | T | 156 | 26.016 | 89.825 | 257.844 | 1.00 | 30.26 | C |
| ATOM | 8097 | CG | GLN | T | 156 | 25.588 | 91.288 | 257.802 | 1.00 | 30.94 | C |
| ATOM | 8100 | CD | GLN | T | 156 | 25.891 | 91.961 | 256.475 | 1.00 | 31.09 | C |
| ATOM | 8101 | OE1 | GLN | T | 156 | 25.928 | 91.313 | 255.427 | 1.00 | 32.88 | O |
| ATOM | 8102 | NE2 | GLN | T | 156 | 26.103 | 93.273 | 256.514 | 1.00 | 31.03 | N |
| ATOM | 8105 | C | GLN | T | 156 | 25.349 | 87.761 | 259.110 | 1.00 | 28.77 | C |
| ATOM | 8106 | O | GLN | T | 156 | 26.090 | 86.858 | 258.716 | 1.00 | 28.10 | O |
| ATOM | 8108 | N | PRO | T | 157 | 24.063 | 87.551 | 259.448 | 1.00 | 28.11 | N |
| ATOM | 8109 | CA | PRO | T | 157 | 23.491 | 86.211 | 259.369 | 1.00 | 27.91 | C |
| ATOM | 8111 | CB | PRO | T | 157 | 22.158 | 86.352 | 260.111 | 1.00 | 27.94 | C |
| ATOM | 8114 | CG | PRO | T | 157 | 21.790 | 87.778 | 259.966 | 1.00 | 28.02 | C |
| ATOM | 8117 | CD | PRO | T | 157 | 23.077 | 88.546 | 259.916 | 1.00 | 28.09 | C |
| ATOM | 8120 | C | PRO | T | 157 | 23.264 | 85.771 | 257.925 | 1.00 | 27.51 | C |
| ATOM | 8121 | O | PRO | T | 157 | 23.246 | 86.606 | 257.016 | 1.00 | 27.87 | O |
| ATOM | 8122 | N | ALA | T | 158 | 23.106 | 84.468 | 257.718 | 1.00 | 26.53 | N |
| ATOM | 8123 | CA | ALA | T | 158 | 22.831 | 83.940 | 256.385 | 1.00 | 27.12 | C |
| ATOM | 8125 | CB | ALA | T | 158 | 23.194 | 82.464 | 256.312 | 1.00 | 27.87 | C |
| ATOM | 8129 | C | ALA | T | 158 | 21.366 | 84.143 | 256.005 | 1.00 | 26.16 | C |
| ATOM | 8130 | O | ALA | T | 158 | 20.499 | 84.269 | 256.874 | 1.00 | 24.55 | O |
| ATOM | 8132 | N | SER | T | 159 | 21.111 | 84.183 | 254.699 | 1.00 | 26.14 | N |
| ATOM | 8133 | CA | SER | T | 159 | 19.755 | 84.261 | 254.144 | 1.00 | 25.62 | C |
| ATOM | 8135 | CB | SER | T | 159 | 19.394 | 85.710 | 253.817 | 1.00 | 26.16 | C |
| ATOM | 8138 | OG | SER | T | 159 | 20.210 | 86.216 | 252.772 | 1.00 | 25.57 | O |
| ATOM | 8140 | C | SER | T | 159 | 19.699 | 83.421 | 252.875 | 1.00 | 25.53 | C |
| ATOM | 8141 | O | SER | T | 159 | 20.713 | 82.851 | 252.472 | 1.00 | 23.78 | O |
| ATOM | 8143 | N | ASN | T | 160 | 18.528 | 83.349 | 252.243 | 1.00 | 25.37 | N |
| ATOM | 8144 | CA | ASN | T | 160 | 18.393 | 82.630 | 250.967 | 1.00 | 26.15 | C |
| ATOM | 8146 | CB | ASN | T | 160 | 16.922 | 82.562 | 250.500 | 1.00 | 25.49 | C |
| ATOM | 8149 | CG | ASN | T | 160 | 16.272 | 83.940 | 250.323 | 1.00 | 25.82 | C |
| ATOM | 8150 | OD1 | ASN | T | 160 | 16.900 | 84.979 | 250.538 | 1.00 | 27.51 | O |
| ATOM | 8151 | ND2 | ASN | T | 160 | 14.999 | 83.943 | 249.940 | 1.00 | 23.50 | N |
| ATOM | 8154 | C | ASN | T | 160 | 19.286 | 83.204 | 249.859 | 1.00 | 26.63 | C |
| ATOM | 8155 | O | ASN | T | 160 | 19.781 | 82.461 | 249.013 | 1.00 | 24.30 | O |
| ATOM | 8157 | N | SER | T | 161 | 19.507 | 84.519 | 249.891 | 1.00 | 27.47 | N |
| ATOM | 8158 | CA | SER | T | 161 | 20.220 | 85.227 | 248.821 | 1.00 | 28.36 | C |
| ATOM | 8160 | CB | SER | T | 161 | 19.539 | 86.575 | 248.552 | 1.00 | 28.25 | C |
| ATOM | 8163 | OG | SER | T | 161 | 19.372 | 87.313 | 249.748 | 1.00 | 29.60 | O |
| ATOM | 8165 | C | SER | T | 161 | 21.722 | 85.449 | 249.070 | 1.00 | 28.99 | C |
| ATOM | 8166 | O | SER | T | 161 | 22.440 | 85.855 | 248.153 | 1.00 | 29.56 | O |
| ATOM | 8168 | N | SER | T | 162 | 22.198 | 85.197 | 250.290 | 1.00 | 29.81 | N |
| ATOM | 8169 | CA | SER | T | 162 | 23.605 | 85.456 | 250.627 | 1.00 | 30.17 | C |
| ATOM | 8171 | CB | SER | T | 162 | 23.823 | 86.952 | 250.891 | 1.00 | 30.17 | C |
| ATOM | 8174 | OG | SER | T | 162 | 23.028 | 87.410 | 251.973 | 1.00 | 29.77 | O |
| ATOM | 8176 | C | SER | T | 162 | 24.114 | 84.646 | 251.823 | 1.00 | 30.42 | C |
| ATOM | 8177 | O | SER | T | 162 | 23.342 | 84.238 | 252.695 | 1.00 | 30.44 | O |
| ATOM | 8179 | N | ASP | T | 163 | 25.427 | 84.431 | 251.847 | 1.00 | 30.81 | N |
| ATOM | 8180 | CA | ASP | T | 163 | 26.085 | 83.661 | 252.900 | 1.00 | 31.76 | C |
| ATOM | 8182 | CB | ASP | T | 163 | 27.407 | 83.084 | 252.373 | 1.00 | 30.27 | C |
| ATOM | 8185 | CG | ASP | T | 163 | 27.931 | 81.924 | 253.213 | 1.00 | 30.14 | C |
| ATOM | 8186 | OD1 | ASP | T | 163 | 27.215 | 81.439 | 254.116 | 1.00 | 29.23 | O |
| ATOM | 8187 | OD2 | ASP | T | 163 | 29.072 | 81.487 | 252.959 | 1.00 | 30.39 | O |
| ATOM | 8188 | C | ASP | T | 163 | 26.355 | 84.549 | 254.116 | 1.00 | 32.78 | C |
| ATOM | 8189 | O | ASP | T | 163 | 26.437 | 85.775 | 253.996 | 1.00 | 32.94 | O |
| ATOM | 8191 | N | ALA | T | 164 | 26.489 | 83.922 | 255.282 | 1.00 | 33.65 | N |
| ATOM | 8192 | CA | ALA | T | 164 | 26.854 | 84.635 | 256.507 | 1.00 | 35.56 | C |
| ATOM | 8194 | CB | ALA | T | 164 | 26.635 | 83.744 | 257.726 | 1.00 | 35.68 | C |
| ATOM | 8198 | C | ALA | T | 164 | 28.308 | 85.109 | 256.455 | 1.00 | 36.88 | C |
| ATOM | 8199 | O | ALA | T | 164 | 29.162 | 84.450 | 255.851 | 1.00 | 36.68 | O |
| ATOM | 8201 | N | ILE | T | 165 | 28.573 | 86.252 | 257.087 | 1.00 | 37.68 | N |
| ATOM | 8202 | CA | ILE | T | 165 | 29.922 | 86.816 | 257.181 | 1.00 | 37.89 | C |
| ATOM | 8204 | CB | ILE | T | 165 | 29.958 | 88.295 | 256.711 | 1.00 | 37.93 | C |
| ATOM | 8206 | CG1 | ILE | T | 165 | 29.415 | 88.416 | 255.281 | 1.00 | 38.14 | C |
| ATOM | 8209 | CD1 | ILE | T | 165 | 29.190 | 89.845 | 254.816 | 1.00 | 38.14 | C |
| ATOM | 8213 | CG2 | ILE | T | 165 | 31.384 | 88.852 | 256.786 | 1.00 | 37.67 | C |
| ATOM | 8217 | C | ILE | T | 165 | 30.405 | 86.719 | 258.629 | 1.00 | 38.11 | C |
| ATOM | 8218 | O | ILE | T | 165 | 29.613 | 86.850 | 259.564 | 1.00 | 37.30 | O |
| ATOM | 8220 | N | CYS | T | 166 | 31.706 | 86.493 | 258.805 | 1.00 | 38.75 | N |
| ATOM | 8221 | CA | CYS | T | 166 | 32.300 | 86.302 | 260.129 | 1.00 | 39.81 | C |
| ATOM | 8223 | CB | CYS | T | 166 | 33.033 | 84.964 | 260.181 | 1.00 | 41.59 | C |
| ATOM | 8226 | SG | CYS | T | 166 | 32.030 | 83.571 | 259.616 | 1.00 | 44.81 | S |
| ATOM | 8228 | C | CYS | T | 166 | 33.262 | 87.434 | 260.487 | 1.00 | 39.70 | C |
| ATOM | 8229 | O | CYS | T | 166 | 33.429 | 88.388 | 259.725 | 1.00 | 39.41 | O |
| ATOM | 8231 | S | SO4 | W | 101 | 23.436 | 90.519 | 154.786 | 1.00 | 34.98 | S |
| ATOM | 8232 | O1 | SO4 | W | 101 | 24.221 | 91.111 | 155.861 | 1.00 | 35.51 | O |
| ATOM | 8233 | O2 | SO4 | W | 101 | 22.405 | 89.675 | 155.374 | 1.00 | 39.55 | O |
| ATOM | 8234 | O3 | SO4 | W | 101 | 24.308 | 89.740 | 153.943 | 1.00 | 27.98 | O |
| ATOM | 8235 | O4 | SO4 | W | 101 | 22.813 | 91.573 | 153.998 | 1.00 | 37.81 | O |
| ATOM | 8236 | O | HOH | W | 102 | 54.770 | 107.250 | 167.229 | 1.00 | 18.07 | O |
| ATOM | 8239 | O | HOH | W | 103 | 25.783 | 79.731 | 188.012 | 1.00 | 19.03 | O |
| ATOM | 8242 | O | HOH | W | 104 | 51.166 | 92.972 | 163.241 | 1.00 | 19.81 | O |
| ATOM | 8245 | O | HOH | W | 105 | 10.348 | 47.286 | 208.660 | 1.00 | 20.45 | O |
| ATOM | 8248 | O | HOH | W | 106 | 52.836 | 101.667 | 160.751 | 1.00 | 28.06 | O |
| ATOM | 8251 | O | HOH | W | 107 | 41.023 | 104.540 | 163.225 | 1.00 | 22.36 | O |
| ATOM | 8254 | O | HOH | W | 108 | 20.822 | 87.787 | 162.673 | 1.00 | 28.40 | O |
| ATOM | 8257 | O | HOH | W | 109 | 41.446 | 99.657 | 162.968 | 1.00 | 22.67 | O |
| ATOM | 8260 | O | HOH | W | 110 | 57.746 | 103.147 | 172.937 | 1.00 | 24.63 | O |
| ATOM | 8263 | O | HOH | W | 111 | 19.283 | 81.375 | 168.922 | 1.00 | 25.86 | O |
| ATOM | 8266 | O | HOH | W | 112 | 29.754 | 85.296 | 154.615 | 1.00 | 21.18 | O |
| ATOM | 8269 | O | HOH | W | 113 | 23.323 | 79.718 | 183.259 | 1.00 | 27.29 | O |
| ATOM | 8272 | O | HOH | W | 114 | 20.378 | 79.074 | 170.287 | 1.00 | 29.25 | O |
| ATOM | 8275 | O | HOH | W | 115 | 54.352 | 108.045 | 175.112 | 1.00 | 27.64 | O |
| ATOM | 8278 | O | HOH | W | 116 | −0.552 | 62.997 | 204.601 | 1.00 | 28.66 | O |
| ATOM | 8281 | O | HOH | W | 117 | 42.770 | 94.541 | 158.217 | 1.00 | 25.15 | O |
| ATOM | 8284 | O | HOH | W | 118 | 50.058 | 91.424 | 160.988 | 1.00 | 27.15 | O |
| ATOM | 8287 | O | HOH | W | 119 | 51.488 | 99.804 | 159.890 | 1.00 | 25.07 | O |
| ATOM | 8290 | O | HOH | W | 120 | 22.574 | 87.045 | 156.033 | 1.00 | 27.43 | O |
| ATOM | 8293 | O | HOH | W | 121 | 51.410 | 97.977 | 157.195 | 1.00 | 30.46 | O |
| ATOM | 8296 | O | HOH | W | 122 | 19.365 | 85.192 | 205.100 | 1.00 | 34.57 | O |
| ATOM | 8299 | O | HOH | W | 123 | 42.099 | 103.383 | 160.947 | 1.00 | 33.36 | O |
| ATOM | 8302 | O | HOH | W | 124 | 26.180 | 76.822 | 192.140 | 1.00 | 36.64 | O |
| ATOM | 8305 | O | HOH | W | 125 | 14.124 | 62.157 | 204.327 | 1.00 | 25.15 | O |
| ATOM | 8308 | O | HOH | W | 126 | 20.504 | 81.744 | 163.170 | 1.00 | 29.14 | O |
| ATOM | 8311 | O | HOH | W | 127 | 57.631 | 109.466 | 173.328 | 1.00 | 35.01 | O |
| ATOM | 8314 | O | HOH | W | 128 | 27.008 | 87.281 | 169.355 | 1.00 | 40.26 | O |
| ATOM | 8317 | O | HOH | W | 129 | 7.234 | 54.883 | 201.343 | 1.00 | 26.63 | O |
| ATOM | 8320 | O | HOH | W | 130 | 33.734 | 90.129 | 162.692 | 1.00 | 34.98 | O |
| ATOM | 8323 | O | HOH | W | 131 | 40.788 | 107.004 | 177.231 | 1.00 | 28.36 | O |
| ATOM | 8326 | O | HOH | W | 132 | 56.672 | 115.058 | 167.550 | 1.00 | 24.24 | O |
| ATOM | 8329 | O | HOH | W | 133 | 9.350 | 64.811 | 199.526 | 1.00 | 28.91 | O |
| ATOM | 8332 | O | HOH | W | 134 | 23.728 | 79.566 | 192.471 | 1.00 | 27.79 | O |
| ATOM | 8335 | O | HOH | W | 135 | 31.380 | 95.653 | 163.805 | 1.00 | 34.18 | O |
| ATOM | 8338 | O | HOH | W | 136 | 41.350 | 88.796 | 175.647 | 1.00 | 36.12 | O |
| ATOM | 8341 | O | HOH | W | 137 | 58.583 | 108.754 | 154.669 | 1.00 | 28.17 | O |
| ATOM | 8344 | O | HOH | W | 138 | 44.907 | 114.900 | 185.320 | 1.00 | 31.04 | O |
| ATOM | 8347 | O | HOH | W | 139 | 30.997 | 81.340 | 156.779 | 1.00 | 28.28 | O |
| ATOM | 8350 | O | HOH | W | 140 | 57.251 | 102.563 | 164.819 | 1.00 | 32.60 | O |
| ATOM | 8353 | O | HOH | W | 141 | 6.053 | 56.262 | 198.535 | 1.00 | 36.10 | O |
| ATOM | 8356 | O | HOH | W | 142 | 19.841 | 57.781 | 218.581 | 1.00 | 33.06 | O |
| ATOM | 8359 | O | HOH | W | 143 | 18.890 | 84.265 | 181.651 | 1.00 | 29.85 | O |
| ATOM | 8362 | O | HOH | W | 144 | 32.412 | 92.346 | 154.587 | 1.00 | 27.81 | O |
| ATOM | 8365 | O | HOH | W | 145 | 18.219 | 78.677 | 188.227 | 1.00 | 31.99 | O |
| ATOM | 8368 | O | HOH | W | 146 | 55.308 | 101.162 | 183.952 | 1.00 | 26.44 | O |
| ATOM | 8371 | O | HOH | W | 147 | 41.971 | 87.983 | 161.987 | 1.00 | 37.04 | O |
| ATOM | 8374 | O | HOH | W | 148 | 32.137 | 96.404 | 167.481 | 1.00 | 34.32 | O |
| ATOM | 8377 | O | HOH | W | 149 | 49.848 | 110.144 | 190.055 | 1.00 | 30.80 | O |
| ATOM | 8380 | O | HOH | W | 150 | 55.676 | 103.630 | 183.521 | 1.00 | 41.02 | O |
| ATOM | 8383 | O | HOH | W | 151 | 22.226 | 89.380 | 179.229 | 1.00 | 31.03 | O |
| ATOM | 8386 | O | HOH | W | 152 | 42.770 | 98.607 | 188.639 | 1.00 | 30.68 | O |
| ATOM | 8389 | O | HOH | W | 153 | 57.466 | 98.957 | 179.802 | 1.00 | 36.15 | O |
| ATOM | 8392 | O | HOH | W | 154 | 5.028 | 70.021 | 204.005 | 1.00 | 44.13 | O |
| ATOM | 8395 | O | HOH | W | 155 | 59.808 | 105.621 | 157.158 | 1.00 | 27.98 | O |
| ATOM | 8398 | O | HOH | W | 156 | 44.458 | 103.036 | 158.185 | 1.00 | 28.58 | O |
| ATOM | 8401 | O | HOH | W | 157 | 10.245 | 83.113 | 209.503 | 1.00 | 36.42 | O |
| ATOM | 8404 | O | HOH | W | 158 | 44.142 | 110.937 | 165.761 | 1.00 | 33.37 | O |
| ATOM | 8407 | O | HOH | W | 159 | 7.849 | 52.554 | 201.971 | 1.00 | 33.60 | O |
| ATOM | 8410 | O | HOH | W | 160 | 2.099 | 61.550 | 202.373 | 1.00 | 30.01 | O |
| ATOM | 8413 | O | HOH | W | 161 | 38.439 | 92.459 | 184.732 | 1.00 | 32.69 | O |
| ATOM | 8416 | O | HOH | W | 162 | 22.985 | 83.629 | 158.326 | 1.00 | 32.76 | O |
| ATOM | 8419 | O | HOH | W | 163 | 31.849 | 80.837 | 194.285 | 1.00 | 37.65 | O |
| ATOM | 8422 | O | HOH | W | 164 | 18.779 | 72.676 | 203.893 | 1.00 | 47.73 | O |
| ATOM | 8425 | O | HOH | W | 165 | 40.121 | 98.726 | 188.070 | 1.00 | 38.05 | O |
| ATOM | 8428 | O | HOH | W | 166 | 13.071 | 68.459 | 195.474 | 1.00 | 36.16 | O |
| ATOM | 8431 | O | HOH | W | 167 | 17.861 | 59.243 | 204.496 | 1.00 | 30.52 | O |
| ATOM | 8434 | O | HOH | W | 168 | 5.250 | 48.682 | 206.105 | 1.00 | 38.61 | O |
| ATOM | 8437 | O | HOH | W | 169 | 6.131 | 44.590 | 198.767 | 1.00 | 29.12 | O |
| ATOM | 8440 | O | HOH | W | 170 | 52.913 | 93.472 | 180.211 | 1.00 | 43.87 | O |
| ATOM | 8443 | O | HOH | W | 171 | 42.906 | 94.798 | 188.940 | 1.00 | 37.25 | O |
| ATOM | 8446 | O | HOH | W | 172 | 14.637 | 74.108 | 196.014 | 1.00 | 36.33 | O |
| ATOM | 8449 | O | HOH | W | 173 | 18.074 | 71.254 | 208.715 | 1.00 | 38.78 | O |
| ATOM | 8452 | O | HOH | W | 174 | 41.404 | 105.303 | 190.918 | 1.00 | 33.98 | O |
| ATOM | 8455 | O | HOH | W | 175 | 20.566 | 95.115 | 160.531 | 1.00 | 49.06 | O |
| ATOM | 8458 | O | HOH | W | 176 | 16.270 | 59.196 | 202.257 | 1.00 | 31.78 | O |
| ATOM | 8461 | O | HOH | W | 177 | 16.598 | 87.359 | 194.605 | 1.00 | 39.68 | O |
| ATOM | 8464 | O | HOH | W | 178 | 16.183 | 78.973 | 184.744 | 1.00 | 31.10 | O |
| ATOM | 8467 | O | HOH | W | 179 | 11.788 | 81.011 | 196.129 | 1.00 | 30.47 | O |
| ATOM | 8470 | O | HOH | W | 180 | 56.277 | 113.757 | 165.505 | 1.00 | 34.03 | O |
| ATOM | 8473 | O | HOH | W | 181 | 27.916 | 79.665 | 170.035 | 1.00 | 37.82 | O |
| ATOM | 8476 | O | HOH | W | 182 | 40.430 | 112.933 | 171.470 | 1.00 | 35.13 | O |
| ATOM | 8479 | O | HOH | W | 183 | 30.586 | 90.200 | 179.381 | 1.00 | 63.17 | O |
| ATOM | 8482 | O | HOH | W | 185 | 37.611 | 94.988 | 154.085 | 1.00 | 32.89 | O |
| ATOM | 8485 | O | HOH | W | 186 | 22.506 | 79.494 | 175.747 | 1.00 | 39.27 | O |
| ATOM | 8488 | O | HOH | W | 187 | 48.583 | 105.727 | 197.523 | 1.00 | 52.75 | O |
| ATOM | 8491 | O | HOH | W | 188 | 8.750 | 62.137 | 199.368 | 1.00 | 41.55 | O |
| ATOM | 8494 | O | HOH | W | 189 | 32.671 | 95.088 | 171.436 | 1.00 | 45.11 | O |
| ATOM | 8497 | O | HOH | W | 190 | 11.463 | 74.312 | 189.893 | 1.00 | 41.06 | O |
| ATOM | 8500 | O | HOH | W | 191 | 35.124 | 102.009 | 165.479 | 1.00 | 34.47 | O |
| ATOM | 8503 | O | HOH | W | 193 | 21.896 | 92.799 | 197.298 | 1.00 | 40.92 | O |
| ATOM | 8506 | O | HOH | W | 194 | 7.314 | 49.390 | 225.196 | 1.00 | 43.66 | O |
| ATOM | 8509 | O | HOH | W | 195 | 32.179 | 103.988 | 179.845 | 1.00 | 39.44 | O |
| ATOM | 8512 | O | HOH | W | 196 | 9.993 | 43.825 | 214.884 | 1.00 | 39.92 | O |
| ATOM | 8515 | O | HOH | W | 197 | 52.368 | 90.695 | 164.264 | 0.33 | 24.43 | O |
| ATOM | 8518 | O | HOH | W | 198 | 0.002 | 60.464 | 205.635 | 0.33 | 23.11 | O |
| ATOM | 8521 | O | HOH | W | 199 | 52.367 | 90.695 | 159.819 | 0.33 | 28.10 | O |
| ATOM | 8524 | O | HOH | W | 200 | 46.307 | 100.028 | 187.959 | 1.00 | 45.36 | O |
| ATOM | 8527 | O | HOH | W | 201 | 12.425 | 74.380 | 204.026 | 1.00 | 34.45 | O |
| ATOM | 8530 | O | HOH | W | 202 | 59.165 | 108.588 | 161.177 | 1.00 | 31.50 | O |
| ATOM | 8533 | O | HOH | W | 203 | 58.724 | 108.163 | 175.517 | 1.00 | 40.30 | O |
| ATOM | 8536 | O | HOH | W | 204 | 47.895 | 91.860 | 159.377 | 1.00 | 37.40 | O |
| ATOM | 8539 | O | HOH | W | 205 | 29.501 | 79.120 | 157.361 | 1.00 | 39.50 | O |
| ATOM | 8542 | O | HOH | W | 206 | 35.079 | 91.104 | 155.012 | 1.00 | 29.96 | O |
| ATOM | 8545 | O | HOH | W | 207 | 22.495 | 77.617 | 177.306 | 1.00 | 50.19 | O |
| ATOM | 8548 | O | HOH | W | 208 | 8.337 | 43.284 | 202.618 | 1.00 | 36.59 | O |
| ATOM | 8551 | O | HOH | W | 209 | 5.442 | 47.969 | 214.237 | 1.00 | 35.58 | O |
| ATOM | 8554 | O | HOH | W | 210 | 0.833 | 65.609 | 197.403 | 1.00 | 40.43 | O |
| ATOM | 8557 | O | HOH | W | 211 | 20.164 | 65.215 | 206.995 | 1.00 | 43.89 | O |
| ATOM | 8560 | O | HOH | W | 212 | 20.074 | 59.716 | 203.316 | 1.00 | 39.93 | O |
| ATOM | 8563 | O | HOH | W | 213 | 27.842 | 92.430 | 190.949 | 1.00 | 36.03 | O |
| ATOM | 8566 | O | HOH | W | 214 | 18.241 | 87.493 | 196.699 | 1.00 | 39.37 | O |
| ATOM | 8569 | O | HOH | W | 215 | 20.219 | 83.549 | 198.012 | 1.00 | 33.33 | O |
| ATOM | 8572 | O | HOH | W | 216 | 33.512 | 107.423 | 176.531 | 1.00 | 44.90 | O |
| ATOM | 8575 | O | HOH | W | 217 | 14.404 | 57.827 | 199.595 | 1.00 | 31.83 | O |
| ATOM | 8578 | O | HOH | W | 218 | 45.797 | 107.926 | 160.727 | 1.00 | 47.97 | O |
| ATOM | 8581 | O | HOH | W | 219 | 2.345 | 56.205 | 234.550 | 1.00 | 42.91 | O |
| ATOM | 8584 | O | HOH | W | 220 | 37.657 | 90.553 | 165.261 | 1.00 | 40.73 | O |
| ATOM | 8587 | O | HOH | W | 221 | 52.278 | 95.771 | 156.150 | 1.00 | 36.34 | O |
| ATOM | 8590 | O | HOH | W | 222 | 27.043 | 89.635 | 149.322 | 1.00 | 46.63 | O |
| ATOM | 8593 | O | HOH | W | 223 | 11.645 | 43.133 | 223.170 | 1.00 | 38.24 | O |
| ATOM | 8596 | O | HOH | W | 224 | 5.644 | 51.309 | 225.407 | 1.00 | 35.87 | O |
| ATOM | 8599 | O | HOH | W | 225 | 11.107 | 47.101 | 216.620 | 1.00 | 38.48 | O |
| ATOM | 8602 | O | HOH | W | 226 | 36.037 | 108.104 | 175.082 | 1.00 | 41.68 | O |
| ATOM | 8605 | O | HOH | W | 227 | 31.887 | 81.919 | 181.843 | 1.00 | 37.49 | O |
| ATOM | 8608 | O | HOH | W | 228 | 24.793 | 86.884 | 148.285 | 1.00 | 33.64 | O |
| ATOM | 8611 | O | HOH | W | 230 | 13.481 | 46.239 | 222.019 | 1.00 | 41.95 | O |
| ATOM | 8614 | O | HOH | W | 232 | 53.369 | 111.775 | 157.875 | 1.00 | 39.60 | O |
| ATOM | 8617 | O | HOH | W | 235 | 41.200 | 87.982 | 157.255 | 1.00 | 44.43 | O |
| ATOM | 8620 | O | HOH | W | 236 | 16.612 | 60.703 | 199.715 | 1.00 | 49.63 | O |
| ATOM | 8623 | O | HOH | W | 238 | 29.873 | 95.897 | 162.001 | 1.00 | 37.66 | O |
| ATOM | 8626 | O | HOH | W | 239 | 32.258 | 100.048 | 178.534 | 1.00 | 38.86 | O |
| ATOM | 8629 | O | HOH | W | 241 | 46.356 | 104.752 | 156.721 | 1.00 | 45.69 | O |
| ATOM | 8632 | O | HOH | W | 242 | 37.136 | 102.994 | 186.046 | 1.00 | 62.70 | O |
| ATOM | 8635 | O | HOH | W | 243 | 48.815 | 112.303 | 191.146 | 1.00 | 42.28 | O |
| ATOM | 8638 | O | HOH | W | 244 | 49.861 | 111.078 | 162.348 | 1.00 | 37.82 | O |
| ATOM | 8641 | O | HOH | W | 245 | 15.180 | 71.707 | 187.444 | 1.00 | 48.45 | O |
| ATOM | 8644 | O | HOH | W | 246 | 12.453 | 62.230 | 229.870 | 1.00 | 39.77 | O |
| ATOM | 8647 | O | HOH | W | 247 | 51.667 | 112.487 | 159.789 | 1.00 | 47.05 | O |
| ATOM | 8650 | O | HOH | W | 248 | 49.601 | 109.381 | 160.322 | 1.00 | 42.54 | O |
| ATOM | 8653 | O | HOH | W | 249 | 43.031 | 106.103 | 160.220 | 1.00 | 46.86 | O |
| ATOM | 8656 | O | HOH | W | 250 | 39.012 | 105.745 | 161.946 | 1.00 | 35.16 | O |
| ATOM | 8659 | O | HOH | W | 251 | 40.527 | 106.556 | 159.585 | 1.00 | 52.88 | O |
| ATOM | 8662 | O | HOH | W | 252 | 36.784 | 103.741 | 161.256 | 1.00 | 47.49 | O |
| ATOM | 8665 | O | HOH | W | 253 | 31.541 | 112.141 | 168.878 | 1.00 | 60.98 | O |
| ATOM | 8668 | O | HOH | W | 254 | 57.441 | 100.204 | 182.407 | 1.00 | 35.79 | O |
| ATOM | 8671 | O | HOH | W | 255 | 58.924 | 102.200 | 183.026 | 1.00 | 46.18 | O |
| ATOM | 8674 | O | HOH | W | 256 | 53.323 | 115.388 | 192.659 | 1.00 | 47.09 | O |
| ATOM | 8677 | O | HOH | W | 257 | 31.090 | 92.765 | 177.359 | 1.00 | 55.44 | O |
| ATOM | 8680 | O | HOH | W | 258 | 57.593 | 105.693 | 175.608 | 1.00 | 42.87 | O |
| ATOM | 8683 | O | HOH | W | 259 | 37.393 | 92.396 | 153.871 | 1.00 | 46.96 | O |
| ATOM | 8686 | O | HOH | W | 260 | 23.675 | 61.131 | 226.789 | 1.00 | 51.61 | O |
| ATOM | 8689 | O | HOH | W | 261 | 17.634 | 52.195 | 206.674 | 1.00 | 44.40 | O |
| ATOM | 8692 | O | HOH | W | 262 | 20.260 | 52.708 | 207.200 | 1.00 | 37.56 | O |
| ATOM | 8695 | O | HOH | W | 263 | 14.074 | 64.076 | 229.512 | 1.00 | 44.70 | O |
| ATOM | 8698 | O | HOH | W | 264 | 9.538 | 65.041 | 230.251 | 1.00 | 49.75 | O |
| ATOM | 8701 | O | HOH | W | 265 | 3.028 | 61.888 | 222.574 | 1.00 | 36.43 | O |
| ATOM | 8704 | O | HOH | W | 266 | 10.023 | 71.460 | 206.573 | 1.00 | 47.90 | O |
| ATOM | 8707 | O | HOH | W | 267 | 23.371 | 68.283 | 210.126 | 1.00 | 50.35 | O |
| ATOM | 8710 | O | HOH | W | 268 | 22.870 | 49.531 | 226.677 | 1.00 | 43.65 | O |
| ATOM | 8713 | O | HOH | W | 269 | 31.904 | 99.251 | 149.957 | 1.00 | 52.04 | O |
| ATOM | 8716 | O | HOH | W | 270 | 32.801 | 84.650 | 150.732 | 1.00 | 48.20 | O |
| ATOM | 8719 | O | HOH | W | 271 | 33.440 | 84.497 | 153.363 | 1.00 | 49.57 | O |
| ATOM | 8722 | O | HOH | W | 272 | 24.654 | 76.881 | 167.263 | 1.00 | 40.34 | O |
| ATOM | 8725 | O | HOH | W | 273 | 27.291 | 80.204 | 167.477 | 1.00 | 44.53 | O |
| ATOM | 8728 | O | HOH | W | 275 | 20.277 | 85.533 | 196.267 | 1.00 | 41.93 | O |
| ATOM | 8731 | O | HOH | W | 276 | 18.279 | 78.264 | 178.803 | 1.00 | 41.09 | O |
| ATOM | 8734 | O | HOH | W | 277 | 17.797 | 79.835 | 174.353 | 1.00 | 39.48 | O |
| ATOM | 8737 | O | HOH | W | 278 | 21.201 | 88.456 | 182.588 | 1.00 | 42.99 | O |
| ATOM | 8740 | O | HOH | W | 279 | 23.028 | 88.833 | 186.953 | 1.00 | 33.82 | O |
| ATOM | 8743 | O | HOH | W | 280 | 25.349 | 87.591 | 184.298 | 1.00 | 40.23 | O |
| ATOM | 8746 | O | HOH | W | 281 | 29.258 | 91.370 | 189.205 | 1.00 | 35.91 | O |
| ATOM | 8749 | O | HOH | W | 282 | 25.496 | 80.174 | 202.241 | 1.00 | 46.81 | O |
| ATOM | 8752 | O | HOH | W | 283 | 20.164 | 69.464 | 191.339 | 1.00 | 46.09 | O |
| ATOM | 8755 | O | HOH | W | 284 | 26.981 | 74.919 | 190.142 | 1.00 | 53.98 | O |
| ATOM | 8758 | O | HOH | W | 285 | 8.072 | 82.895 | 198.325 | 1.00 | 37.18 | O |
| ATOM | 8761 | O | HOH | W | 286 | 10.593 | 78.310 | 189.044 | 1.00 | 46.87 | O |

TABLE 10

| | | |
|---|---|---|
| HEADER | ---- | XX-XXX-XX xxxx |
| COMPND | --- | |

| REMARK | 3 | | |
|---|---|---|---|
| REMARK | 3 | REFINEMENT. | |
| REMARK | 3 | PROGRAM | : REFMAC 5.2.0005 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.41 |
| REMARK | 3 | RESOLUTION RANGE LOW | (ANGSTROMS) | : 30.00 |
| REMARK | 3 | DATA CUTOFF | (SIGMA(F)) | : NONE |
| REMARK | 3 | COMPLETENESS FOR RANGE | (%) | : 99.86 |
| REMARK | 3 | NUMBER OF REFLECTIONS | | : 19882 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.21248 |
| REMARK | 3 | R VALUE | (WORKING SET) | : 0.20825 |

| | | | | |
|---|---|---|---|---|
| REMARK | 3 | FREE R VALUE | | : 0.25110 |
| REMARK | 3 | FREE R VALUE TEST SET SIZE | (%) | : 10.0 |
| REMARK | 3 | FREE R VALUE TEST SET COUNT | | : 2221 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : 25 |
| REMARK | 3 | BIN RESOLUTION RANGE HIGH | | : 2.406 |
| REMARK | 3 | BIN RESOLUTION RANGE LOW | | : 2.456 |
| REMARK | 3 | REFLECTION IN BIN | (WORKING SET) | : 1144 |
| REMARK | 3 | BIN COMPLETENESS | (WORKING + TEST) (%) | : 99.92 |
| REMARK | 3 | BIN R VALUE | (WORKING SET) | : 0.276 |

| | | | |
|---|---|---|---|
| REMARK | 3 | BIN FREE R VALUE SET COUNT | : 133 |
| REMARK | 3 | BIN FREE R VALUE | : 0.357 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS : 2175 | | |
| REMARK | 3 | | | |
| REMARK | 3 | B VALUES. | | |
| REMARK | 3 | FROM WILSON PLOT | (A**2) : | NULL |
| REMARK | 3 | MEAN B VALUE | (OVERALL, A**2) : | 38.975 |

| REMARK | 3 | OVERALL ANISOTROPIC B VALUE. | | |
|---|---|---|---|---|
| REMARK | 3 | B11 (A**2) : 1.91 | | |
| REMARK | 3 | B22 (A**2) : 1.91 | | |
| REMARK | 3 | B33 (A**2) : −2.87 | | |
| REMARK | 3 | B12 (A**2) : 0.96 | | |
| REMARK | 3 | B13 (A**2) : 0.00 | | |
| REMARK | 3 | B23 (A**2) : 0.00 | | |
| REMARK | 3 | | | |
| REMARK | 3 | ESTIMATED OVERALL COORDINATE ERROR. | | |
| REMARK | 3 | ESU BASED ON R VALUE | (A): | 0.229 |
| REMARK | 3 | ESU BASED ON FREE R VALUE | (A): | 0.207 |
| REMARK | 3 | ESU BASED ON MAXIMUM LIKELIHOOD | (A): | 0.136 |
| REMARK | 3 | ESU FOR B VALUES BASED ON MAXIMUM LIKELIHOOD | (A**2): | 11.211 |
| REMARK | 3 | | | |

| | | | |
|---|---|---|---|
| REMARK | 3 | CORRELATION COEFFICIENTS. | |
| REMARK | 3 | CORRELATION COEFFICIENT FO-FC | : 0.941 |
| REMARK | 3 | CORRELATION COEFFICIENT FO-FC FREE | : 0.913 |
| REMARK | 3 | | |

| | | | | | | |
|---|---|---|---|---|---|---|
| REMARK | 3 | RMS DEVIATIONS FROM IDEAL VALUES | | COUNT | RMS | WEIGHT |
| REMARK | 3 | BOND LENGTHS REFINED ATOMS | (A): | 2143 | ; 0.007 | ; 0.022 |
| REMARK | 3 | BOND LENGTHS OTHERS | (A): | 1831 | ; 0.001 | ; 0.020 |
| REMARK | 3 | BOND ANGLES REFINED ATOMS | (DEGREES): | 2910 | ; 1.109 | ; 1.974 |
| REMARK | 3 | BOND ANGLES OTHERS | (DEGREES): | 4310 | ; 0.694 | ; 3.000 |
| REMARK | 3 | TORSION ANGLES, PERIOD 1 | (DEGREES): | 264 | ; 5.866 | ; 5.000 |
| REMARK | 3 | TORSION ANGLES, PERIOD 2 | (DEGREES): | 96 | ;35.790 | ;25.104 |
| REMARK | 3 | TORSION ANGLES, PERIOD 3 | (DEGREES): | 360 | ;14.019 | ;15.000 |
| REMARK | 3 | TORSION ANGLES, PERIOD 4 | (DEGREES): | 10 | ;13.332 | ;15.000 |
| REMARK | 3 | CHIRAL-CENTER RESTRAINTS | (A**3): | 320 | ; 0.067 | ; 0.200 |
| REMARK | 3 | GENERAL PLANES REFINED ATOMS | (A): | 2359 | ; 0.003 | ; 0.020 |
| REMARK | 3 | GENERAL PLANES OTHERS | (A): | 393 | ; 0.001 | ; 0.020 |
| REMARK | 3 | NON-BONDED CONTACTS REFINED ATOMS | (A): | 343 | ; 0.172 | ; 0.200 |
| REMARK | 3 | NON-BONDED CONTACTS OTHERS | (A): | 1692 | ; 0.168 | ; 0.200 |
| REMARK | 3 | NON-BONDED TORSION REFINED ATOMS | (A): | 976 | ; 0.170 | ; 0.200 |
| REMARK | 3 | NON-BONDED TORSION OTHERS | (A): | 1284 | ; 0.080 | ; 0.200 |
| REMARK | 3 | H-BOND (X...Y) REFINED ATOMS | (A): | 84 | ; 0.155 | ; 0.200 |
| REMARK | 3 | SYMMETRY VDW REFINED ATOMS | (A): | 9 | ; 0.124 | ; 0.200 |
| REMARK | 3 | SYMMETRY VDW OTHERS | (A): | 47 | ; 0.158 | ; 0.200 |
| REMARK | 3 | SYMMETRY H-BOND REFINED ATOMS | (A): | 12 | ; 0.328 | ; 0.200 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 1708 | ; 2.242 | ; 2.500 |
| REMARK | 3 | MAIN-CHAIN BOND OTHER ATOMS | (A**2): | 537 | ; 0.368 | ; 2.500 |
| REMARK | 3 | MAIN-CHAIN ANGLE REFINED ATOMS | (A**2): | 2155 | ; 2.877 | ; 5.000 |
| REMARK | 3 | SIDE-CHAIN BOND REFINED ATOMS | (A**2): | 938 | ; 1.909 | ; 2.500 |
| REMARK | 3 | SIDE-CHAIN ANGLE REFINED ATOMS | (A**2): | 755 | ; 2.852 | ; 5.000 |
| REMARK | 3 | | | | | |

| REMARK | 3 | NCS RESTRAINTS STATISTICS | | |
|---|---|---|---|---|
| REMARK | 3 | NUMBER OF NCS GROUPS: NULL | | |
| REMARK | 3 | | | |
| REMARK | 3 | | | |
| REMARK | 3 | TLS DETAILS | | |
| REMARK | 3 | NUMBER OF TLS GROUPS : 4 | | |
| REMARK | 3 | ATOM RECORD CONTAINS RESIDUAL B FACTORS ONLY | | |
| REMARK | 3 | | | |
| REMARK | 3 | TLS GROUP : 1 | | |
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | F 58 | F 200 |

| REMARK | 3 | ORIGIN FOR THE GROUP (A): −7.8785 50.6023 72.1994 | | | |
|---|---|---|---|---|---|
| REMARK | 3 | T TENSOR | | | |
| REMARK | 3 | T11: | −0.1062 T22: | −0.1850 | |
| REMARK | 3 | T33: | −0.0686 T12: | 0.0015 | |
| REMARK | 3 | T13: | 0.0020 T23: | −0.0049 | |
| REMARK | 3 | L TENSOR | | | |
| REMARK | 3 | L11: | 3.8381 L22: | 2.1796 | |
| REMARK | 3 | L33: | 4.3252 L12: | −0.8241 | |
| REMARK | 3 | L13: | −2.1881 L23: | 0.5823 | |
| REMARK | 3 | S TENSOR | | | |
| REMARK | 3 | S11: | −0.2276 S12: | −0.0799 S13: | −0.4435 |
| REMARK | 3 | S21: | 0.1453 S22: | 0.0664 S23: | 0.2325 |
| REMARK | 3 | S31: | 0.4190 S32: | −0.0636 S33: | 0.1612 |
| REMARK | 3 | | | | |

| REMARK | 3 | TLS GROUP : 2 | | |
|---|---|---|---|---|
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | R 29 | R 108 |

| REMARK | 3 | ORIGIN FOR THE GROUP (A): 13.7010 41.2586 66.6269 | | | |
|---|---|---|---|---|---|
| REMARK | 3 | T TENSOR | | | |
| REMARK | 3 | T11: | −0.0682 T22: | −0.1533 | |
| REMARK | 3 | T33: | −0.0925 T12: | 0.0389 | |
| REMARK | 3 | T13: | 0.0388 T23: | 0.0193 | |
| REMARK | 3 | L TENSOR | | | |
| REMARK | 3 | L11: | 1.9807 L22: | 2.6417 | |
| REMARK | 3 | L33: | 11.0623 L12: | −0.2957 | |
| REMARK | 3 | L13: | 2.3891 L23: | 1.0837 | |
| REMARK | 3 | S TENSOR | | | |
| REMARK | 3 | S11: | 0.1067 S12: | 0.2259 S13: | −0.2228 |
| REMARK | 3 | S21: | −0.2702 S22: | −0.2205 S23: | −0.0924 |
| REMARK | 3 | S31: | 0.1546 S32: | 0.3839 S33: | 0.1138 |
| REMARK | 3 | | | | |

| REMARK | 3 | TLS GROUP : 3 | | |
|---|---|---|---|---|
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | R 109 | R 140 |

| REMARK | 3 | ORIGIN FOR THE GROUP (A): 11.6952 43.2929 95.7162 | | | |
|---|---|---|---|---|---|
| REMARK | 3 | T TENSOR | | | |
| REMARK | 3 | T11: | 0.1892 T22: | −0.1001 | |
| REMARK | 3 | T33: | −0.0550 T12: | 0.0496 | |
| REMARK | 3 | T13: | 0.0457 T23: | 0.0097 | |
| REMARK | 3 | L TENSOR | | | |
| REMARK | 3 | L11: | 3.7438 L22: | 10.2790 | |
| REMARK | 3 | L33: | 17.8915 L12: | 3.7855 | |
| REMARK | 3 | L13: | −2.0543 L23: | −12.4767 | |
| REMARK | 3 | S TENSOR | | | |
| REMARK | 3 | S11: | 0.3741 S12: | 0.4377 S13: | 0.5374 |
| REMARK | 3 | S21: | 1.1423 S22: | 0.1023 S23: | 0.4310 |
| REMARK | 3 | S31: | −1.4776 S32: | −0.6530 S33: | −0.4764 |
| REMARK | 3 | | | | |

| REMARK | 3 | TLS GROUP : 4 | | |
|---|---|---|---|---|
| REMARK | 3 | NUMBER OF COMPONENTS GROUP : 1 | | |
| REMARK | 3 | COMPONENTS | C SSSEQI TO | C SSSEQI |
| REMARK | 3 | RESIDUE RANGE : | R 141 | R 200 |

| REMARK | 3 | ORIGIN FOR THE GROUP (A): 8.7716 33.7127 113.0385 | | | |
|---|---|---|---|---|---|
| REMARK | 3 | T TENSOR | | | |
| REMARK | 3 | T11: | 0.3210 T22: | 0.1111 | |
| REMARK | 3 | T33: | −0.1255 T12: | −0.0831 | |
| REMARK | 3 | T13: | 0.0000 T23: | −0.0175 | |
| REMARK | 3 | L TENSOR | | | |
| REMARK | 3 | L11: | 12.7405 L22: | 12.4875 | |
| REMARK | 3 | L33: | 34.5604 L12: | 2.0421 | |
| REMARK | 3 | L13: | −12.3548 L23: | −14.4159 | |
| REMARK | 3 | S TENSOR | | | |
| REMARK | 3 | S11: | 0.1582 S12: | −1.6300 S13: | −0.6502 |
| REMARK | 3 | S21: | 1.3331 S22: | −0.4932 S23: | 0.1028 |
| REMARK | 3 | S31: | −1.0266 S32: | 1.5312 S33: | 0.3350 |
| REMARK | 3 | | | | |
| REMARK | 3 | | | | |

| | | | |
|---|---|---|---|
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS : 1.40 | |
| REMARK | 3 | ION PROBE RADIUS : 0.80 | |
| REMARK | 3 | SHRINKAGE RADIUS : 0.80 | |
| REMARK | 3 | | |
| REMARK | 3 | OTHER REFINEMENT REMARKS: | |
| REMARK | 3 | HYDROGENS HAVE BEEN ADDED IN THE RIDING POSITIONS | |
| REMARK | 3 | | |
| SSBOND | 1 | CYS F 97 CYS F 181 | |
| LINK | | C1 NAG F 200 1.439 ND2 ASN F 152 | NAG- |
| ASN | | | |

| | | | | | |
|---|---|---|---|---|---|
| SSBOND | 2 | CYS R | 31 | CYS R | 42 |
| SSBOND | 3 | CYS R | 43 | CYS R | 56 |
| SSBOND | 4 | CYS R | 46 | CYS R | 64 |
| SSBOND | 5 | CYS R | 67 | CYS R | 81 |
| SSBOND | 6 | CYS R | 84 | CYS R | 99 |
| SSBOND | 7 | CYS R | 87 | CYS R | 107 |
| SSBOND | 8 | CYS R | 109 | CYS R | 125 |
| SSBOND | 9 | CYS R | 128 | CYS R | 141 |
| SSBOND | 10 | CYS R | 147 | CYS R | 166 |

| | | |
|---|---|---|
| LINK | C1 NAG R 200 1.439 ND2 ASN R 160 | NAG- |
| ASN | | |

| | | |
|---|---|---|
| MODRES | NAG F 200 NAG-b-D | RENAME |
| MODRES | NAG R 200 NAG-b-D | RENAME |

| | |
|---|---|
| CRYST1 | 111.903 111.903 233.238 90.00 90.00 120.00 H 3 2 |

| | | | | |
|---|---|---|---|---|
| SCALE1 | 0.008936 | 0.005159 | 0.000000 | 0.00000 |
| SCALE2 | 0.000000 | 0.010319 | 0.000000 | 0.00000 |
| SCALE3 | 0.000000 | 0.000000 | 0.004287 | 0.00000 |

| | | | | | | | | | | | |
|---|---|---|---|---|---|---|---|---|---|---|---|
| ATOM | 1 | N | ARG | F | 58 | −8.066 | 59.372 | 53.307 | 1.00 | 58.49 | N |
| ATOM | 2 | CA | ARG | F | 58 | −6.756 | 58.742 | 52.936 | 1.00 | 56.65 | C |
| ATOM | 4 | CB | ARG | F | 58 | −6.019 | 59.599 | 51.900 | 1.00 | 58.42 | C |
| ATOM | 7 | CG | ARG | F | 58 | −4.970 | 58.832 | 51.105 | 1.00 | 61.43 | C |
| ATOM | 10 | CD | ARG | F | 58 | −4.191 | 59.757 | 50.175 | 1.00 | 64.49 | C |
| ATOM | 13 | NE | ARG | F | 58 | −5.012 | 60.284 | 49.084 | 1.00 | 65.39 | N |
| ATOM | 15 | CZ | ARG | F | 58 | −4.701 | 61.350 | 48.342 | 1.00 | 68.08 | C |
| ATOM | 16 | NH1 | ARG | F | 58 | −3.580 | 62.036 | 48.560 | 1.00 | 68.21 | N |
| ATOM | 19 | NH2 | ARG | F | 58 | −5.522 | 61.739 | 47.369 | 1.00 | 68.35 | N |
| ATOM | 22 | C | ARG | F | 58 | −5.858 | 58.516 | 54.153 | 1.00 | 51.17 | C |
| ATOM | 23 | O | ARG | F | 58 | −5.102 | 57.545 | 54.196 | 1.00 | 50.16 | O |
| ATOM | 27 | N | ILE | F | 59 | −5.934 | 59.422 | 55.128 | 1.00 | 47.64 | N |
| ATOM | 28 | CA | ILE | F | 59 | −5.190 | 59.288 | 56.381 | 1.00 | 47.31 | C |
| ATOM | 30 | CB | ILE | F | 59 | −5.233 | 60.597 | 57.225 | 1.00 | 47.48 | C |
| ATOM | 32 | CG1 | ILE | F | 59 | −4.504 | 61.735 | 56.493 | 1.00 | 49.70 | C |
| ATOM | 35 | CD1 | ILE | F | 59 | −3.009 | 61.526 | 56.283 | 1.00 | 50.48 | C |
| ATOM | 39 | CG2 | ILE | F | 59 | −4.638 | 60.392 | 58.617 | 1.00 | 46.65 | C |
| ATOM | 43 | C | ILE | F | 59 | −5.754 | 58.132 | 57.198 | 1.00 | 42.96 | C |
| ATOM | 44 | O | ILE | F | 59 | −6.961 | 58.015 | 57.362 | 1.00 | 42.46 | O |
| ATOM | 46 | N | GLN | F | 60 | −4.863 | 57.277 | 57.695 | 1.00 | 42.46 | N |
| ATOM | 47 | CA | GLN | F | 60 | −5.253 | 56.142 | 58.520 | 1.00 | 40.15 | C |
| ATOM | 49 | CB | GLN | F | 60 | −4.151 | 55.082 | 58.527 | 1.00 | 37.56 | C |
| ATOM | 52 | CG | GLN | F | 60 | −3.888 | 54.446 | 57.174 | 1.00 | 37.51 | C |
| ATOM | 55 | CD | GLN | F | 60 | −4.732 | 53.211 | 56.889 | 1.00 | 36.17 | C |
| ATOM | 56 | OE1 | GLN | F | 60 | −5.080 | 52.450 | 57.797 | 1.00 | 35.68 | O |
| ATOM | 57 | NE2 | GLN | F | 60 | −5.032 | 52.987 | 55.610 | 1.00 | 32.67 | N |
| ATOM | 60 | C | GLN | F | 60 | −5.557 | 56.621 | 59.940 | 1.00 | 39.06 | C |
| ATOM | 61 | O | GLN | F | 60 | −4.647 | 56.944 | 60.714 | 1.00 | 34.97 | O |
| ATOM | 63 | N | SER | F | 61 | −6.847 | 56.693 | 60.262 | 1.00 | 38.09 | N |
| ATOM | 64 | CA | SER | F | 61 | −7.284 | 57.127 | 61.585 | 1.00 | 38.03 | C |
| ATOM | 66 | CB | SER | F | 61 | −7.350 | 58.659 | 61.669 | 1.00 | 36.83 | C |
| ATOM | 69 | OG | SER | F | 61 | −8.551 | 59.159 | 61.115 | 1.00 | 36.77 | O |
| ATOM | 71 | C | SER | F | 61 | −8.634 | 56.525 | 61.947 | 1.00 | 37.56 | C |
| ATOM | 72 | O | SER | F | 61 | −9.398 | 56.108 | 61.072 | 1.00 | 38.68 | O |
| ATOM | 74 | N | ILE | F | 62 | −8.911 | 56.471 | 63.247 | 1.00 | 37.77 | N |
| ATOM | 75 | CA | ILE | F | 62 | −10.161 | 55.912 | 63.744 | 1.00 | 39.42 | C |
| ATOM | 77 | CB | ILE | F | 62 | −10.074 | 54.360 | 63.840 | 1.00 | 39.72 | C |
| ATOM | 79 | CG1 | ILE | F | 62 | −11.465 | 53.740 | 64.025 | 1.00 | 41.38 | C |
| ATOM | 82 | CD1 | ILE | F | 62 | −11.472 | 52.234 | 63.905 | 1.00 | 40.54 | C |
| ATOM | 86 | CG2 | ILE | F | 62 | −9.144 | 53.920 | 64.956 | 1.00 | 39.35 | C |
| ATOM | 90 | C | ILE | F | 62 | −10.530 | 56.498 | 65.106 | 1.00 | 40.39 | C |
| ATOM | 91 | O | ILE | F | 62 | −9.652 | 56.785 | 65.926 | 1.00 | 39.04 | O |
| ATOM | 93 | N | LYS | F | 63 | −11.833 | 56.680 | 65.319 | 1.00 | 41.91 | N |
| ATOM | 94 | CA | LYS | F | 63 | −12.407 | 56.956 | 66.643 | 1.00 | 41.77 | C |
| ATOM | 96 | CB | LYS | F | 63 | −13.314 | 58.175 | 66.592 | 1.00 | 42.73 | C |
| ATOM | 99 | CG | LYS | F | 63 | −13.748 | 58.699 | 67.951 | 1.00 | 45.72 | C |
| ATOM | 102 | CD | LYS | F | 63 | −14.860 | 59.732 | 67.772 | 1.00 | 46.90 | C |
| ATOM | 105 | CE | LYS | F | 63 | −14.845 | 60.802 | 68.856 | 1.00 | 49.93 | C |
| ATOM | 108 | NZ | LYS | F | 63 | −15.995 | 61.747 | 68.699 | 1.00 | 51.44 | N |
| ATOM | 112 | C | LYS | F | 63 | −13.214 | 55.728 | 67.095 | 1.00 | 40.14 | C |
| ATOM | 113 | O | LYS | F | 63 | −14.175 | 55.325 | 66.438 | 1.00 | 39.40 | O |
| ATOM | 115 | N | VAL | F | 64 | −12.799 | 55.137 | 68.207 | 1.00 | 38.33 | N |
| ATOM | 116 | CA | VAL | F | 64 | −13.477 | 53.995 | 68.804 | 1.00 | 40.38 | C |
| ATOM | 118 | CB | VAL | F | 64 | −12.462 | 52.848 | 69.111 | 1.00 | 41.99 | C |
| ATOM | 120 | CG1 | VAL | F | 64 | −13.034 | 51.833 | 70.096 | 1.00 | 41.32 | C |
| ATOM | 124 | CG2 | VAL | F | 64 | −12.029 | 52.168 | 67.820 | 1.00 | 42.76 | C |
| ATOM | 128 | C | VAL | F | 64 | −14.183 | 54.424 | 70.094 | 1.00 | 37.52 | C |
| ATOM | 129 | O | VAL | F | 64 | −13.645 | 55.208 | 70.881 | 1.00 | 36.68 | O |
| ATOM | 131 | N | GLN | F | 65 | −15.389 | 53.907 | 70.297 | 1.00 | 36.59 | N |
| ATOM | 132 | CA | GLN | F | 65 | −16.088 | 54.045 | 71.576 | 1.00 | 39.52 | C |
| ATOM | 134 | CB | GLN | F | 65 | −17.436 | 54.755 | 71.387 | 1.00 | 37.77 | C |
| ATOM | 137 | CG | GLN | F | 65 | −17.252 | 56.218 | 71.016 | 1.00 | 37.67 | C |
| ATOM | 140 | CD | GLN | F | 65 | −18.518 | 56.920 | 70.597 | 1.00 | 37.85 | C |
| ATOM | 141 | OE1 | GLN | F | 65 | −19.499 | 56.299 | 70.188 | 1.00 | 38.69 | O |
| ATOM | 142 | NE2 | GLN | F | 65 | −18.494 | 58.236 | 70.678 | 1.00 | 36.91 | N |
| ATOM | 145 | C | GLN | F | 65 | −16.280 | 52.674 | 72.198 | 1.00 | 40.08 | C |
| ATOM | 146 | O | GLN | F | 65 | −16.169 | 51.651 | 71.519 | 1.00 | 41.65 | O |
| ATOM | 148 | N | PHE | F | 66 | −16.537 | 52.664 | 73.499 | 1.00 | 40.06 | N |
| ATOM | 149 | CA | PHE | F | 66 | −16.828 | 51.433 | 74.222 | 1.00 | 39.35 | C |
| ATOM | 151 | CB | PHE | F | 66 | −15.537 | 50.727 | 74.659 | 1.00 | 38.78 | C |
| ATOM | 154 | CG | PHE | F | 66 | −14.565 | 51.609 | 75.405 | 1.00 | 38.58 | C |
| ATOM | 155 | CD1 | PHE | F | 66 | −13.628 | 52.369 | 74.721 | 1.00 | 38.38 | C |
| ATOM | 157 | CE1 | PHE | F | 66 | −12.731 | 53.176 | 75.417 | 1.00 | 39.28 | C |
| ATOM | 159 | CZ | PHE | F | 66 | −12.765 | 53.218 | 76.822 | 1.00 | 37.27 | C |
| ATOM | 161 | CE2 | PHE | F | 66 | −13.682 | 52.461 | 77.503 | 1.00 | 37.24 | C |
| ATOM | 163 | CD2 | PHE | F | 66 | −14.577 | 51.659 | 76.800 | 1.00 | 39.05 | C |
| ATOM | 165 | C | PHE | F | 66 | −17.737 | 51.737 | 75.413 | 1.00 | 41.76 | C |
| ATOM | 166 | O | PHE | F | 66 | −17.486 | 52.678 | 76.179 | 1.00 | 40.06 | O |
| ATOM | 168 | N | THR | F | 67 | −18.822 | 50.970 | 75.522 | 1.00 | 42.72 | N |
| ATOM | 169 | CA | THR | F | 67 | −19.793 | 51.128 | 76.606 | 1.00 | 43.44 | C |
| ATOM | 171 | CB | THR | F | 67 | −21.075 | 51.850 | 76.116 | 1.00 | 44.64 | C |
| ATOM | 173 | OG1 | THR | F | 67 | −21.825 | 50.992 | 75.244 | 1.00 | 45.40 | O |
| ATOM | 175 | CG2 | THR | F | 67 | −20.720 | 53.126 | 75.364 | 1.00 | 47.40 | C |
| ATOM | 179 | C | THR | F | 67 | −20.168 | 49.786 | 77.257 | 1.00 | 44.06 | C |
| ATOM | 180 | O | THR | F | 67 | −20.254 | 49.695 | 78.481 | 1.00 | 44.75 | O |
| ATOM | 182 | N | GLU | F | 68 | −20.387 | 48.756 | 76.443 | 1.00 | 44.26 | N |
| ATOM | 183 | CA | GLU | F | 68 | −20.831 | 47.455 | 76.936 | 1.00 | 45.71 | C |
| ATOM | 185 | CB | GLU | F | 68 | −21.557 | 46.701 | 75.820 | 1.00 | 46.87 | C |
| ATOM | 188 | CG | GLU | F | 68 | −22.364 | 45.498 | 76.290 | 1.00 | 48.57 | C |
| ATOM | 191 | CD | GLU | F | 68 | −22.823 | 44.591 | 75.152 | 1.00 | 50.54 | C |
| ATOM | 192 | OE1 | GLU | F | 68 | −22.578 | 44.919 | 73.972 | 1.00 | 51.01 | O |
| ATOM | 193 | OE2 | GLU | F | 68 | −23.433 | 43.538 | 75.448 | 1.00 | 54.16 | O |
| ATOM | 194 | C | GLU | F | 68 | −19.655 | 46.615 | 77.454 | 1.00 | 45.18 | C |
| ATOM | 195 | O | GLU | F | 68 | −18.704 | 46.364 | 76.728 | 1.00 | 45.34 | O |
| ATOM | 197 | N | TYR | F | 69 | −19.739 | 46.181 | 78.708 | 1.00 | 44.43 | N |
| ATOM | 198 | CA | TYR | F | 69 | −18.733 | 45.318 | 79.321 | 1.00 | 46.97 | C |
| ATOM | 200 | CB | TYR | F | 69 | −18.407 | 45.824 | 80.729 | 1.00 | 43.39 | C |
| ATOM | 203 | CG | TYR | F | 69 | −17.190 | 45.189 | 81.380 | 1.00 | 43.27 | C |
| ATOM | 204 | CD1 | TYR | F | 69 | −15.902 | 45.527 | 80.973 | 1.00 | 43.51 | C |
| ATOM | 206 | CE1 | TYR | F | 69 | −14.782 | 44.962 | 81.577 | 1.00 | 43.05 | C |
| ATOM | 208 | CZ | TYR | F | 69 | −14.948 | 44.058 | 82.612 | 1.00 | 42.56 | C |
| ATOM | 209 | OH | TYR | F | 69 | −13.847 | 43.495 | 83.217 | 1.00 | 43.08 | O |
| ATOM | 211 | CE2 | TYR | F | 69 | −16.215 | 43.709 | 83.036 | 1.00 | 42.19 | C |
| ATOM | 213 | CD2 | TYR | F | 69 | −17.327 | 44.276 | 82.424 | 1.00 | 43.05 | C |
| ATOM | 215 | C | TYR | F | 69 | −19.260 | 43.883 | 79.383 | 1.00 | 48.46 | C |
| ATOM | 216 | O | TYR | F | 69 | −20.242 | 43.603 | 80.073 | 1.00 | 45.35 | O |
| ATOM | 218 | N | LYS | F | 70 | −18.611 | 42.986 | 78.645 | 1.00 | 52.61 | N |
| ATOM | 219 | CA | LYS | F | 70 | −18.967 | 41.568 | 78.642 | 1.00 | 56.94 | C |
| ATOM | 221 | CB | LYS | F | 70 | −18.932 | 41.012 | 77.219 | 1.00 | 58.20 | C |
| ATOM | 224 | CG | LYS | F | 70 | −20.142 | 41.396 | 76.375 | 1.00 | 60.12 | C |
| ATOM | 227 | CD | LYS | F | 70 | −20.294 | 40.472 | 75.169 | 1.00 | 61.22 | C |
| ATOM | 230 | CE | LYS | F | 70 | −20.930 | 39.132 | 75.552 | 1.00 | 63.13 | C |
| ATOM | 233 | NZ | LYS | F | 70 | −20.662 | 38.073 | 74.533 | 1.00 | 62.18 | N |
| ATOM | 237 | C | LYS | F | 70 | −18.018 | 40.780 | 79.543 | 1.00 | 59.04 | C |
| ATOM | 238 | O | LYS | F | 70 | −16.821 | 41.067 | 79.593 | 1.00 | 57.20 | O |
| ATOM | 240 | N | LYS | F | 71 | −18.564 | 39.781 | 80.239 | 1.00 | 64.32 | N |
| ATOM | 241 | CA | LYS | F | 71 | −17.832 | 39.027 | 81.268 | 1.00 | 65.32 | C |
| ATOM | 243 | CB | LYS | F | 71 | −18.672 | 37.837 | 81.756 | 1.00 | 67.52 | C |
| ATOM | 246 | CG | LYS | F | 71 | −18.043 | 37.043 | 82.907 | 1.00 | 67.88 | C |
| ATOM | 249 | CD | LYS | F | 71 | −18.702 | 35.678 | 83.091 | 1.00 | 68.02 | C |
| ATOM | 252 | CE | LYS | F | 71 | −17.885 | 34.788 | 84.019 | 1.00 | 68.00 | C |
| ATOM | 255 | NZ | LYS | F | 71 | −18.663 | 33.604 | 84.481 | 1.00 | 67.74 | N |
| ATOM | 259 | C | LYS | F | 71 | −16.457 | 38.542 | 80.797 | 1.00 | 67.15 | C |
| ATOM | 260 | O | LYS | F | 71 | −15.427 | 38.988 | 81.314 | 1.00 | 68.33 | O |
| ATOM | 262 | N | GLU | F | 72 | −16.442 | 37.643 | 79.816 | 1.00 | 66.64 | N |
| ATOM | 263 | CA | GLU | F | 72 | −15.192 | 37.049 | 79.335 | 1.00 | 66.21 | C |
| ATOM | 265 | CB | GLU | F | 72 | −15.304 | 35.518 | 79.319 | 1.00 | 68.61 | C |
| ATOM | 268 | CG | GLU | F | 72 | −15.722 | 34.931 | 80.671 | 1.00 | 69.08 | C |
| ATOM | 271 | CD | GLU | F | 72 | −15.503 | 33.433 | 80.769 | 1.00 | 69.95 | C |
| ATOM | 272 | OE1 | GLU | F | 72 | −14.520 | 32.930 | 80.181 | 1.00 | 72.23 | O |
| ATOM | 273 | OE2 | GLU | F | 72 | −16.310 | 32.758 | 81.447 | 1.00 | 70.17 | O |
| ATOM | 274 | C | GLU | F | 72 | −14.804 | 37.596 | 77.961 | 1.00 | 64.57 | C |
| ATOM | 275 | O | GLU | F | 72 | −14.531 | 36.845 | 77.023 | 1.00 | 65.25 | O |
| ATOM | 277 | N | LYS | F | 73 | −14.778 | 38.920 | 77.864 | 1.00 | 62.67 | N |
| ATOM | 278 | CA | LYS | F | 73 | −14.353 | 39.619 | 76.647 | 1.00 | 59.75 | C |
| ATOM | 280 | CB | LYS | F | 73 | −15.445 | 39.537 | 75.567 | 1.00 | 62.78 | C |
| ATOM | 283 | CG | LYS | F | 73 | −14.977 | 39.916 | 74.149 | 1.00 | 63.42 | C |
| ATOM | 286 | CD | LYS | F | 73 | −15.953 | 39.408 | 73.078 | 1.00 | 63.02 | C |
| ATOM | 289 | CE | LYS | F | 73 | −15.671 | 40.002 | 71.693 | 1.00 | 63.65 | C |
| ATOM | 292 | NZ | LYS | F | 73 | −14.435 | 39.461 | 71.037 | 1.00 | 63.28 | N |
| ATOM | 296 | C | LYS | F | 73 | −13.979 | 41.084 | 76.924 | 1.00 | 55.49 | C |
| ATOM | 297 | O | LYS | F | 73 | −13.140 | 41.653 | 76.222 | 1.00 | 53.60 | O |
| ATOM | 299 | N | GLY | F | 74 | −14.607 | 41.689 | 77.935 | 1.00 | 51.53 | N |
| ATOM | 300 | CA | GLY | F | 74 | −14.312 | 43.066 | 78.339 | 1.00 | 47.77 | C |
| ATOM | 303 | C | GLY | F | 74 | −15.177 | 44.063 | 77.595 | 1.00 | 40.13 | C |
| ATOM | 304 | O | GLY | F | 74 | −16.209 | 43.704 | 77.041 | 1.00 | 36.92 | O |
| ATOM | 306 | N | PHE | F | 75 | −14.752 | 45.321 | 77.584 | 1.00 | 41.78 | N |
| ATOM | 307 | CA | PHE | F | 75 | −15.457 | 46.371 | 76.841 | 1.00 | 41.77 | C |
| ATOM | 309 | CB | PHE | F | 75 | −14.864 | 47.754 | 77.132 | 1.00 | 38.29 | C |
| ATOM | 312 | CG | PHE | F | 75 | −15.282 | 48.322 | 78.464 | 1.00 | 38.25 | C |
| ATOM | 313 | CD1 | PHE | F | 75 | −16.576 | 48.796 | 78.657 | 1.00 | 38.72 | C |
| ATOM | 315 | CE1 | PHE | F | 75 | −16.974 | 49.315 | 79.887 | 1.00 | 38.08 | C |
| ATOM | 317 | CZ | PHE | F | 75 | −16.075 | 49.357 | 80.937 | 1.00 | 38.52 | C |
| ATOM | 319 | CE2 | PHE | F | 75 | −14.778 | 48.883 | 80.758 | 1.00 | 37.82 | C |
| ATOM | 321 | CD2 | PHE | F | 75 | −14.391 | 48.371 | 79.526 | 1.00 | 37.77 | C |
| ATOM | 323 | C | PHE | F | 75 | −15.469 | 46.096 | 75.333 | 1.00 | 41.91 | C |
| ATOM | 324 | O | PHE | F | 75 | −14.445 | 45.734 | 74.737 | 1.00 | 40.98 | O |
| ATOM | 326 | N | ILE | F | 76 | −16.645 | 46.261 | 74.735 | 1.00 | 42.39 | N |
| ATOM | 327 | CA | ILE | F | 76 | −16.841 | 46.017 | 73.308 | 1.00 | 41.90 | C |
| ATOM | 329 | CB | ILE | F | 76 | −18.297 | 45.575 | 73.000 | 1.00 | 41.54 | C |
| ATOM | 331 | CG1 | ILE | F | 76 | −18.687 | 44.355 | 73.843 | 1.00 | 41.53 | C |
| ATOM | 334 | CD1 | ILE | F | 76 | −17.820 | 43.138 | 73.635 | 1.00 | 42.80 | C |
| ATOM | 338 | CG2 | ILE | F | 76 | −18.478 | 45.281 | 71.511 | 1.00 | 41.15 | C |
| ATOM | 342 | C | ILE | F | 76 | −16.507 | 47.294 | 72.545 | 1.00 | 39.95 | C |
| ATOM | 343 | O | ILE | F | 76 | −17.090 | 48.345 | 72.813 | 1.00 | 38.39 | O |
| ATOM | 345 | N | LEU | F | 77 | −15.564 | 47.196 | 71.608 | 1.00 | 38.25 | N |
| ATOM | 346 | CA | LEU | F | 77 | −15.127 | 48.347 | 70.824 | 1.00 | 38.89 | C |
| ATOM | 348 | CB | LEU | F | 77 | −13.690 | 48.160 | 70.327 | 1.00 | 38.78 | C |
| ATOM | 351 | CG | LEU | F | 77 | −12.561 | 47.944 | 71.341 | 1.00 | 35.86 | C |
| ATOM | 353 | CD1 | LEU | F | 77 | −11.226 | 48.028 | 70.622 | 1.00 | 35.83 | C |
| ATOM | 357 | CD2 | LEU | F | 77 | −12.611 | 48.940 | 72.499 | 1.00 | 34.56 | C |
| ATOM | 361 | C | LEU | F | 77 | −16.045 | 48.568 | 69.627 | 1.00 | 39.58 | C |
| ATOM | 362 | O | LEU | F | 77 | −16.250 | 47.665 | 68.820 | 1.00 | 39.30 | O |
| ATOM | 364 | N | THR | F | 78 | −16.584 | 49.783 | 69.521 | 1.00 | 42.21 | N |
| ATOM | 365 | CA | THR | F | 78 | −17.463 | 50.178 | 68.422 | 1.00 | 40.01 | C |
| ATOM | 367 | CB | THR | F | 78 | −18.889 | 50.526 | 68.940 | 1.00 | 39.74 | C |
| ATOM | 369 | OG1 | THR | F | 78 | −18.830 | 51.653 | 69.823 | 1.00 | 41.89 | O |
| ATOM | 371 | CG2 | THR | F | 78 | −19.497 | 49.343 | 69.697 | 1.00 | 37.63 | C |
| ATOM | 375 | C | THR | F | 78 | −16.869 | 51.370 | 67.653 | 1.00 | 38.43 | C |
| ATOM | 376 | O | THR | F | 78 | −16.140 | 52.179 | 68.213 | 1.00 | 36.20 | O |
| ATOM | 378 | N | SER | F | 79 | −17.165 | 51.436 | 66.358 | 1.00 | 41.65 | N |
| ATOM | 379 | CA | SER | F | 79 | −16.787 | 52.557 | 65.502 | 1.00 | 44.07 | C |
| ATOM | 381 | CB | SER | F | 79 | −15.357 | 52.401 | 64.995 | 1.00 | 44.28 | C |
| ATOM | 384 | OG | SER | F | 79 | −15.222 | 51.262 | 64.172 | 1.00 | 44.96 | O |
| ATOM | 386 | C | SER | F | 79 | −17.746 | 52.615 | 64.323 | 1.00 | 47.37 | C |
| ATOM | 387 | O | SER | F | 79 | −18.565 | 51.716 | 64.150 | 1.00 | 46.09 | O |
| ATOM | 389 | N | GLN | F | 80 | −17.641 | 53.671 | 63.515 | 1.00 | 51.57 | N |
| ATOM | 390 | CA | GLN | F | 80 | −18.480 | 53.820 | 62.316 | 1.00 | 49.85 | C |
| ATOM | 392 | CB | GLN | F | 80 | −18.670 | 55.296 | 61.965 | 1.00 | 49.14 | C |
| ATOM | 395 | CG | GLN | F | 80 | −19.511 | 55.527 | 60.715 | 1.00 | 51.31 | C |
| ATOM | 398 | CD | GLN | F | 80 | −19.700 | 56.996 | 60.402 | 1.00 | 52.92 | C |
| ATOM | 399 | OE1 | GLN | F | 80 | −20.142 | 57.771 | 61.257 | 1.00 | 56.02 | O |
| ATOM | 400 | NE2 | GLN | F | 80 | −19.375 | 57.389 | 59.168 | 1.00 | 50.15 | N |
| ATOM | 403 | C | GLN | F | 80 | −17.862 | 53.077 | 61.128 | 1.00 | 48.98 | C |
| ATOM | 404 | O | GLN | F | 80 | −16.691 | 53.274 | 60.808 | 1.00 | 46.25 | O |
| ATOM | 406 | N | LYS | F | 81 | −18.669 | 52.250 | 60.464 | 1.00 | 50.59 | N |
| ATOM | 407 | CA | LYS | F | 81 | −18.194 | 51.402 | 59.364 | 1.00 | 52.32 | C |
| ATOM | 409 | CB | LYS | F | 81 | −18.972 | 50.077 | 59.338 | 1.00 | 57.01 | C |
| ATOM | 412 | CG | LYS | F | 81 | −19.090 | 49.381 | 60.706 | 1.00 | 59.64 | C |
| ATOM | 415 | CD | LYS | F | 81 | −17.738 | 49.227 | 61.407 | 1.00 | 59.75 | C |
| ATOM | 418 | CE | LYS | F | 81 | −17.887 | 48.643 | 62.811 | 1.00 | 59.83 | C |
| ATOM | 421 | NZ | LYS | F | 81 | −16.628 | 48.734 | 63.622 | 1.00 | 58.60 | N |
| ATOM | 425 | C | LYS | F | 81 | −18.279 | 52.082 | 57.995 | 1.00 | 50.75 | C |
| ATOM | 426 | O | LYS | F | 81 | −17.569 | 51.691 | 57.068 | 1.00 | 49.69 | O |
| ATOM | 428 | N | GLU | F | 82 | −19.147 | 53.084 | 57.868 | 1.00 | 49.62 | N |
| ATOM | 429 | CA | GLU | F | 82 | −19.250 | 53.857 | 56.635 | 1.00 | 49.12 | C |
| ATOM | 431 | CB | GLU | F | 82 | −20.528 | 54.700 | 56.608 | 1.00 | 49.43 | C |
| ATOM | 434 | CG | GLU | F | 82 | −21.813 | 53.899 | 56.476 | 1.00 | 52.96 | C |
| ATOM | 437 | CD | GLU | F | 82 | −22.457 | 53.566 | 57.809 | 1.00 | 54.62 | C |
| ATOM | 438 | OE1 | GLU | F | 82 | −23.699 | 53.455 | 57.830 | 1.00 | 54.41 | O |
| ATOM | 439 | OE2 | GLU | F | 82 | −21.737 | 53.423 | 58.827 | 1.00 | 55.37 | O |
| ATOM | 440 | C | GLU | F | 82 | −18.042 | 54.775 | 56.477 | 1.00 | 45.82 | C |
| ATOM | 441 | O | GLU | F | 82 | −17.730 | 55.555 | 57.374 | 1.00 | 43.51 | O |
| ATOM | 443 | N | ASP | F | 83 | −17.397 | 54.687 | 55.316 | 1.00 | 47.86 | N |
| ATOM | 444 | CA | ASP | F | 83 | −16.183 | 55.449 | 54.991 | 1.00 | 50.62 | C |
| ATOM | 446 | CB | ASP | F | 83 | −16.423 | 56.968 | 55.056 | 1.00 | 54.79 | C |
| ATOM | 449 | CG | ASP | F | 83 | −17.686 | 57.403 | 54.334 | 1.00 | 58.50 | C |
| ATOM | 450 | OD1 | ASP | F | 83 | −17.959 | 56.888 | 53.227 | 1.00 | 58.88 | O |
| ATOM | 451 | OD2 | ASP | F | 83 | −18.398 | 58.276 | 54.881 | 1.00 | 60.11 | O |
| ATOM | 452 | C | ASP | F | 83 | −14.993 | 55.086 | 55.885 | 1.00 | 47.45 | C |
| ATOM | 453 | O | ASP | F | 83 | −14.049 | 55.869 | 55.998 | 1.00 | 48.00 | O |
| ATOM | 455 | N | GLU | F | 84 | −15.020 | 53.904 | 56.498 | 1.00 | 45.77 | N |
| ATOM | 456 | CA | GLU | F | 84 | −13.957 | 53.517 | 57.423 | 1.00 | 48.89 | C |
| ATOM | 458 | CB | GLU | F | 84 | −14.390 | 52.343 | 58.313 | 1.00 | 49.19 | C |
| ATOM | 461 | CG | GLU | F | 84 | −14.338 | 50.968 | 57.658 | 1.00 | 49.79 | C |
| ATOM | 464 | CD | GLU | F | 84 | −14.811 | 49.859 | 58.575 | 1.00 | 50.13 | C |
| ATOM | 465 | OE1 | GLU | F | 84 | −14.868 | 50.077 | 59.806 | 1.00 | 49.46 | O |
| ATOM | 466 | OE2 | GLU | F | 84 | −15.116 | 48.759 | 58.061 | 1.00 | 53.55 | O |
| ATOM | 467 | C | GLU | F | 84 | −12.672 | 53.182 | 56.667 | 1.00 | 48.42 | C |
| ATOM | 468 | O | GLU | F | 84 | −12.712 | 52.620 | 55.574 | 1.00 | 47.87 | O |
| ATOM | 470 | N | ILE | F | 85 | −11.537 | 53.553 | 57.248 | 1.00 | 50.15 | N |
| ATOM | 471 | CA | ILE | F | 85 | −10.228 | 53.205 | 56.681 | 1.00 | 50.05 | C |
| ATOM | 473 | CB | ILE | F | 85 | −9.362 | 54.469 | 56.397 | 1.00 | 53.68 | C |
| ATOM | 475 | CG1 | ILE | F | 85 | −8.097 | 54.080 | 55.634 | 1.00 | 56.23 | C |
| ATOM | 478 | CD1 | ILE | F | 85 | −7.302 | 55.253 | 55.122 | 1.00 | 56.60 | C |
| ATOM | 482 | CG2 | ILE | F | 85 | −9.000 | 55.203 | 57.674 | 1.00 | 55.72 | C |
| ATOM | 486 | C | ILE | F | 85 | −9.505 | 52.204 | 57.585 | 1.00 | 47.36 | C |
| ATOM | 487 | O | ILE | F | 85 | −8.795 | 51.316 | 57.097 | 1.00 | 47.31 | O |
| ATOM | 489 | N | MET | F | 86 | −9.696 | 52.356 | 58.895 | 1.00 | 45.05 | N |
| ATOM | 490 | CA | MET | F | 86 | −9.250 | 51.385 | 59.887 | 1.00 | 44.95 | C |
| ATOM | 492 | CB | MET | F | 86 | −8.426 | 52.063 | 60.988 | 1.00 | 40.72 | C |
| ATOM | 495 | CG | MET | F | 86 | −7.153 | 52.745 | 60.496 | 1.00 | 41.06 | C |
| ATOM | 498 | SD | MET | F | 86 | −6.251 | 53.638 | 61.782 | 1.00 | 41.36 | S |
| ATOM | 499 | CE | MET | F | 86 | −5.497 | 52.293 | 62.677 | 1.00 | 44.06 | C |
| ATOM | 503 | C | MET | F | 86 | −10.475 | 50.685 | 60.489 | 1.00 | 45.01 | C |
| ATOM | 504 | O | MET | F | 86 | −11.491 | 51.318 | 60.779 | 1.00 | 42.88 | O |
| ATOM | 506 | N | LYS | F | 87 | −10.356 | 49.374 | 60.673 | 1.00 | 45.98 | N |
| ATOM | 507 | CA | LYS | F | 87 | −11.472 | 48.529 | 61.089 | 1.00 | 47.85 | C |
| ATOM | 509 | CB | LYS | F | 87 | −11.612 | 47.347 | 60.125 | 1.00 | 48.40 | C |
| ATOM | 512 | CG | LYS | F | 87 | −11.612 | 47.755 | 58.668 | 1.00 | 51.87 | C |
| ATOM | 515 | CD | LYS | F | 87 | −12.057 | 46.624 | 57.759 | 1.00 | 53.25 | C |
| ATOM | 518 | CE | LYS | F | 87 | −12.035 | 47.053 | 56.289 | 1.00 | 54.37 | C |
| ATOM | 521 | NZ | LYS | F | 87 | −12.567 | 45.984 | 55.380 | 1.00 | 53.08 | N |
| ATOM | 525 | C | LYS | F | 87 | −11.291 | 47.999 | 62.511 | 1.00 | 43.85 | C |
| ATOM | 526 | O | LYS | F | 87 | −10.167 | 47.914 | 63.022 | 1.00 | 35.73 | O |
| ATOM | 528 | N | VAL | F | 88 | −12.415 | 47.656 | 63.136 | 1.00 | 42.45 | N |
| ATOM | 529 | CA | VAL | F | 88 | −12.418 | 46.890 | 64.374 | 1.00 | 44.24 | C |
| ATOM | 531 | CB | VAL | F | 88 | −13.441 | 47.432 | 65.404 | 1.00 | 44.22 | C |
| ATOM | 533 | CG1 | VAL | F | 88 | −13.551 | 46.495 | 66.613 | 1.00 | 43.36 | C |
| ATOM | 537 | CG2 | VAL | F | 88 | −13.045 | 48.838 | 65.852 | 1.00 | 45.09 | C |
| ATOM | 541 | C | VAL | F | 88 | −12.736 | 45.439 | 64.010 | 1.00 | 41.47 | C |
| ATOM | 542 | O | VAL | F | 88 | −13.716 | 45.162 | 63.321 | 1.00 | 39.18 | O |
| ATOM | 544 | N | GLN | F | 89 | −11.888 | 44.528 | 64.470 | 1.00 | 41.70 | N |
| ATOM | 545 | CA | GLN | F | 89 | −12.020 | 43.114 | 64.168 | 1.00 | 43.90 | C |
| ATOM | 547 | CB | GLN | F | 89 | −11.235 | 42.785 | 62.891 | 1.00 | 45.86 | C |
| ATOM | 550 | CG | GLN | F | 89 | −11.924 | 41.790 | 61.981 | 1.00 | 50.07 | C |
| ATOM | 553 | CD | GLN | F | 89 | −11.014 | 41.245 | 60.887 | 1.00 | 52.30 | C |
| ATOM | 554 | OE1 | GLN | F | 89 | −10.088 | 41.923 | 60.424 | 1.00 | 53.74 | O |
| ATOM | 555 | NE2 | GLN | F | 89 | −11.284 | 40.009 | 60.461 | 1.00 | 56.24 | N |
| ATOM | 558 | C | GLN | F | 89 | −11.480 | 42.326 | 65.364 | 1.00 | 40.92 | C |
| ATOM | 559 | O | GLN | F | 89 | −10.417 | 42.655 | 65.886 | 1.00 | 40.25 | O |
| ATOM | 561 | N | ASP | F | 90 | −12.216 | 41.304 | 65.795 | 1.00 | 40.89 | N |
| ATOM | 562 | CA | ASP | F | 90 | −11.901 | 40.547 | 67.017 | 1.00 | 43.03 | C |
| ATOM | 564 | CB | ASP | F | 90 | −10.654 | 39.672 | 66.826 | 1.00 | 49.16 | C |
| ATOM | 567 | CG | ASP | F | 90 | −10.765 | 38.737 | 65.631 | 1.00 | 52.07 | C |
| ATOM | 568 | OD1 | ASP | F | 90 | −11.844 | 38.145 | 65.423 | 1.00 | 51.49 | O |
| ATOM | 569 | OD2 | ASP | F | 90 | −9.761 | 38.592 | 64.903 | 1.00 | 57.57 | O |
| ATOM | 570 | C | ASP | F | 90 | −11.721 | 41.469 | 68.228 | 1.00 | 41.24 | C |
| ATOM | 571 | O | ASP | F | 90 | −10.806 | 41.281 | 69.035 | 1.00 | 40.11 | O |
| ATOM | 573 | N | ASN | F | 91 | −12.608 | 42.458 | 68.334 | 1.00 | 39.16 | N |
| ATOM | 574 | CA | ASN | F | 91 | −12.598 | 43.458 | 69.405 | 1.00 | 38.71 | C |
| ATOM | 576 | CB | ASN | F | 91 | −12.992 | 42.814 | 70.738 | 1.00 | 39.20 | C |
| ATOM | 579 | CG | ASN | F | 91 | −13.626 | 43.805 | 71.697 | 1.00 | 39.68 | C |
| ATOM | 580 | OD1 | ASN | F | 91 | −14.369 | 44.691 | 71.283 | 1.00 | 40.03 | O |
| ATOM | 581 | ND2 | ASN | F | 91 | −13.338 | 43.657 | 72.982 | 1.00 | 39.93 | N |
| ATOM | 584 | C | ASN | F | 91 | −11.277 | 44.219 | 69.545 | 1.00 | 39.16 | C |
| ATOM | 585 | O | ASN | F | 91 | −10.863 | 44.589 | 70.650 | 1.00 | 36.99 | O |
| ATOM | 587 | N | SER | F | 92 | −10.627 | 44.468 | 68.413 | 1.00 | 41.70 | N |
| ATOM | 588 | CA | SER | F | 92 | −9.360 | 45.187 | 68.392 | 1.00 | 39.32 | C |
| ATOM | 590 | CB | SER | F | 92 | −8.202 | 44.198 | 68.523 | 1.00 | 39.89 | C |
| ATOM | 593 | OG | SER | F | 92 | −8.016 | 43.467 | 67.331 | 1.00 | 42.53 | O |
| ATOM | 595 | C | SER | F | 92 | −9.233 | 46.012 | 67.109 | 1.00 | 38.46 | C |
| ATOM | 596 | O | SER | F | 92 | −9.865 | 45.707 | 66.106 | 1.00 | 38.35 | O |
| ATOM | 598 | N | VAL | F | 93 | −8.420 | 47.063 | 67.165 | 1.00 | 39.14 | N |
| ATOM | 599 | CA | VAL | F | 93 | −8.184 | 47.940 | 66.025 | 1.00 | 37.98 | C |
| ATOM | 601 | CB | VAL | F | 93 | −7.822 | 49.370 | 66.493 | 1.00 | 38.25 | C |
| ATOM | 603 | CG1 | VAL | F | 93 | −7.409 | 50.241 | 65.305 | 1.00 | 38.90 | C |
| ATOM | 607 | CG2 | VAL | F | 93 | −8.988 | 49.998 | 67.258 | 1.00 | 37.55 | C |
| ATOM | 611 | C | VAL | F | 93 | −7.041 | 47.400 | 65.157 | 1.00 | 36.72 | C |
| ATOM | 612 | O | VAL | F | 93 | −5.888 | 47.333 | 65.595 | 1.00 | 34.73 | O |
| ATOM | 614 | N | ILE | F | 94 | −7.363 | 47.064 | 63.912 | 1.00 | 36.49 | N |
| ATOM | 615 | CA | ILE | F | 94 | −6.402 | 46.467 | 62.989 | 1.00 | 37.34 | C |
| ATOM | 617 | CB | ILE | F | 94 | −7.128 | 45.646 | 61.883 | 1.00 | 38.13 | C |
| ATOM | 619 | CG1 | ILE | F | 94 | −8.108 | 44.639 | 62.507 | 1.00 | 38.13 | C |
| ATOM | 622 | CD1 | ILE | F | 94 | −7.483 | 43.674 | 63.488 | 1.00 | 37.39 | C |
| ATOM | 626 | CG2 | ILE | F | 94 | −6.124 | 44.918 | 60.971 | 1.00 | 36.93 | C |
| ATOM | 630 | C | ILE | F | 94 | −5.492 | 47.544 | 62.370 | 1.00 | 37.38 | C |
| ATOM | 631 | O | ILE | F | 94 | −5.963 | 48.471 | 61.720 | 1.00 | 42.04 | O |
| ATOM | 633 | N | ILE | F | 95 | −4.187 | 47.405 | 62.584 | 1.00 | 36.11 | N |
| ATOM | 634 | CA | ILE | F | 95 | −3.196 | 48.312 | 62.011 | 1.00 | 37.69 | C |
| ATOM | 636 | CB | ILE | F | 95 | −2.009 | 48.496 | 62.970 | 1.00 | 41.60 | C |
| ATOM | 638 | CG1 | ILE | F | 95 | −2.483 | 49.305 | 64.188 | 1.00 | 45.37 | C |
| ATOM | 641 | CD1 | ILE | F | 95 | −1.769 | 48.947 | 65.412 | 1.00 | 50.43 | C |
| ATOM | 645 | CG2 | ILE | F | 95 | −0.837 | 49.218 | 62.282 | 1.00 | 38.97 | C |
| ATOM | 649 | C | ILE | F | 95 | −2.738 | 47.841 | 60.627 | 1.00 | 37.92 | C |
| ATOM | 650 | O | ILE | F | 95 | −2.242 | 46.729 | 60.466 | 1.00 | 37.73 | O |
| ATOM | 652 | N | ASN | F | 96 | −2.945 | 48.714 | 59.640 | 1.00 | 37.94 | N |
| ATOM | 653 | CA | ASN | F | 96 | −2.704 | 48.430 | 58.232 | 1.00 | 36.20 | C |
| ATOM | 655 | CB | ASN | F | 96 | −3.769 | 49.120 | 57.372 | 1.00 | 37.20 | C |
| ATOM | 658 | CG | ASN | F | 96 | −5.179 | 48.753 | 57.783 | 1.00 | 35.98 | C |
| ATOM | 659 | OD1 | ASN | F | 96 | −5.471 | 47.584 | 58.027 | 1.00 | 37.56 | O |
| ATOM | 660 | ND2 | ASN | F | 96 | −6.063 | 49.751 | 57.859 | 1.00 | 29.93 | N |
| ATOM | 663 | C | ASN | F | 96 | −1.354 | 48.941 | 57.781 | 1.00 | 35.53 | C |
| ATOM | 664 | O | ASN | F | 96 | −0.721 | 48.330 | 56.916 | 1.00 | 35.15 | O |
| ATOM | 666 | N | CYS | F | 97 | −0.933 | 50.070 | 58.362 | 1.00 | 37.19 | N |
| ATOM | 667 | CA | CYS | F | 97 | 0.253 | 50.802 | 57.914 | 1.00 | 38.30 | C |
| ATOM | 669 | CB | CYS | F | 97 | −0.138 | 52.180 | 57.367 | 1.00 | 36.39 | C |
| ATOM | 672 | SG | CYS | F | 97 | −1.366 | 52.119 | 56.061 | 1.00 | 38.99 | S |
| ATOM | 674 | C | CYS | F | 97 | 1.275 | 50.982 | 59.029 | 1.00 | 36.30 | C |
| ATOM | 675 | O | CYS | F | 97 | 0.921 | 51.304 | 60.164 | 1.00 | 33.31 | O |
| ATOM | 677 | N | ASP | F | 98 | 2.542 | 50.782 | 58.680 | 1.00 | 35.28 | N |
| ATOM | 678 | CA | ASP | F | 98 | 3.647 | 50.977 | 59.602 | 1.00 | 35.43 | C |
| ATOM | 680 | CB | ASP | F | 98 | 4.959 | 50.469 | 58.998 | 1.00 | 34.00 | C |
| ATOM | 683 | CG | ASP | F | 98 | 4.960 | 48.984 | 58.754 | 1.00 | 33.68 | C |
| ATOM | 684 | OD1 | ASP | F | 98 | 3.993 | 48.306 | 59.155 | 1.00 | 32.61 | O |
| ATOM | 685 | OD2 | ASP | F | 98 | 5.944 | 48.495 | 58.159 | 1.00 | 32.93 | O |
| ATOM | 686 | C | ASP | F | 98 | 3.822 | 52.449 | 59.921 | 1.00 | 35.77 | C |
| ATOM | 687 | O | ASP | F | 98 | 3.575 | 53.320 | 59.083 | 1.00 | 35.55 | O |
| ATOM | 689 | N | GLY | F | 99 | 4.263 | 52.723 | 61.140 | 1.00 | 37.33 | N |
| ATOM | 690 | CA | GLY | F | 99 | 4.666 | 54.072 | 61.507 | 1.00 | 35.62 | C |
| ATOM | 693 | C | GLY | F | 99 | 4.248 | 54.419 | 62.908 | 1.00 | 35.92 | C |
| ATOM | 694 | O | GLY | F | 99 | 3.818 | 53.555 | 63.690 | 1.00 | 33.73 | O |
| ATOM | 696 | N | PHE | F | 100 | 4.388 | 55.701 | 63.216 | 1.00 | 35.54 | N |
| ATOM | 697 | CA | PHE | F | 100 | 3.990 | 56.237 | 64.497 | 1.00 | 35.24 | C |
| ATOM | 699 | CB | PHE | F | 100 | 4.676 | 57.581 | 64.769 | 1.00 | 34.17 | C |
| ATOM | 702 | CG | PHE | F | 100 | 6.170 | 57.500 | 64.854 | 1.00 | 31.96 | C |
| ATOM | 703 | CD1 | PHE | F | 100 | 6.782 | 56.746 | 65.835 | 1.00 | 30.75 | C |
| ATOM | 705 | CE1 | PHE | F | 100 | 8.168 | 56.684 | 65.916 | 1.00 | 33.50 | C |
| ATOM | 707 | CZ | PHE | F | 100 | 8.946 | 57.392 | 65.011 | 1.00 | 33.89 | C |
| ATOM | 709 | CE2 | PHE | F | 100 | 8.344 | 58.150 | 64.033 | 1.00 | 32.40 | C |
| ATOM | 711 | CD2 | PHE | F | 100 | 6.966 | 58.206 | 63.960 | 1.00 | 33.75 | C |
| ATOM | 713 | C | PHE | F | 100 | 2.489 | 56.437 | 64.480 | 1.00 | 35.94 | C |
| ATOM | 714 | O | PHE | F | 100 | 1.905 | 56.782 | 63.443 | 1.00 | 38.22 | O |
| ATOM | 716 | N | TYR | F | 101 | 1.871 | 56.209 | 65.634 | 1.00 | 36.53 | N |
| ATOM | 717 | CA | TYR | F | 101 | 0.453 | 56.455 | 65.817 | 1.00 | 36.10 | C |
| ATOM | 719 | CB | TYR | F | 101 | −0.314 | 55.133 | 65.959 | 1.00 | 36.31 | C |
| ATOM | 722 | CG | TYR | F | 101 | −0.537 | 54.455 | 64.627 | 1.00 | 34.69 | C |
| ATOM | 723 | CD1 | TYR | F | 101 | 0.461 | 53.693 | 64.036 | 1.00 | 35.56 | C |
| ATOM | 725 | CE1 | TYR | F | 101 | 0.262 | 53.081 | 62.799 | 1.00 | 36.62 | C |
| ATOM | 727 | CZ | TYR | F | 101 | −0.939 | 53.253 | 62.147 | 1.00 | 36.30 | C |
| ATOM | 728 | OH | TYR | F | 101 | −1.163 | 52.672 | 60.918 | 1.00 | 35.42 | O |
| ATOM | 730 | CE2 | TYR | F | 101 | −1.937 | 54.018 | 62.723 | 1.00 | 37.30 | C |
| ATOM | 732 | CD2 | TYR | F | 101 | −1.732 | 54.614 | 63.943 | 1.00 | 33.54 | C |
| ATOM | 734 | C | TYR | F | 101 | 0.269 | 57.321 | 67.042 | 1.00 | 37.21 | C |
| ATOM | 735 | O | TYR | F | 101 | 0.746 | 56.979 | 68.121 | 1.00 | 36.84 | O |
| ATOM | 737 | N | LEU | F | 102 | −0.397 | 58.456 | 66.850 | 1.00 | 36.20 | N |
| ATOM | 738 | CA | LEU | F | 102 | −0.878 | 59.280 | 67.945 | 1.00 | 36.19 | C |
| ATOM | 740 | CB | LEU | F | 102 | −1.182 | 60.687 | 67.440 | 1.00 | 34.28 | C |
| ATOM | 743 | CG | LEU | F | 102 | −1.729 | 61.717 | 68.426 | 1.00 | 37.33 | C |
| ATOM | 745 | CD1 | LEU | F | 102 | −0.829 | 61.894 | 69.663 | 1.00 | 37.81 | C |
| ATOM | 749 | CD2 | LEU | F | 102 | −1.894 | 63.031 | 67.690 | 1.00 | 35.94 | C |
| ATOM | 753 | C | LEU | F | 102 | −2.136 | 58.628 | 68.511 | 1.00 | 38.38 | C |
| ATOM | 754 | O | LEU | F | 102 | −3.129 | 58.478 | 67.796 | 1.00 | 36.62 | O |
| ATOM | 756 | N | ILE | F | 103 | −2.086 | 58.249 | 69.792 | 1.00 | 38.83 | N |
| ATOM | 757 | CA | ILE | F | 103 | −3.161 | 57.489 | 70.432 | 1.00 | 37.78 | C |
| ATOM | 759 | CB | ILE | F | 103 | −2.675 | 56.087 | 70.880 | 1.00 | 39.67 | C |
| ATOM | 761 | CG1 | ILE | F | 103 | −2.124 | 55.306 | 69.683 | 1.00 | 40.77 | C |
| ATOM | 764 | CD1 | ILE | F | 103 | −1.476 | 54.005 | 70.036 | 1.00 | 41.44 | C |
| ATOM | 768 | CG2 | ILE | F | 103 | −3.812 | 55.314 | 71.550 | 1.00 | 39.60 | C |
| ATOM | 772 | C | ILE | F | 103 | −3.704 | 58.242 | 71.639 | 1.00 | 38.09 | C |
| ATOM | 773 | O | ILE | F | 103 | −2.935 | 58.646 | 72.509 | 1.00 | 38.29 | O |
| ATOM | 775 | N | SER | F | 104 | −5.029 | 58.424 | 71.672 | 1.00 | 40.41 | N |
| ATOM | 776 | CA | SER | F | 104 | −5.748 | 59.048 | 72.787 | 1.00 | 38.58 | C |
| ATOM | 778 | CB | SER | F | 104 | −6.564 | 60.250 | 72.276 | 1.00 | 38.71 | C |
| ATOM | 781 | OG | SER | F | 104 | −7.335 | 60.875 | 73.301 | 1.00 | 35.24 | O |
| ATOM | 783 | C | SER | F | 104 | −6.670 | 57.990 | 73.435 | 1.00 | 39.78 | C |
| ATOM | 784 | O | SER | F | 104 | −7.412 | 57.302 | 72.734 | 1.00 | 35.29 | O |
| ATOM | 786 | N | LEU | F | 105 | −6.592 | 57.849 | 74.760 | 1.00 | 41.17 | N |
| ATOM | 787 | CA | LEU | F | 105 | −7.488 | 56.967 | 75.524 | 1.00 | 41.22 | C |
| ATOM | 789 | CB | LEU | F | 105 | −6.713 | 55.790 | 76.116 | 1.00 | 42.77 | C |
| ATOM | 792 | CG | LEU | F | 105 | −7.493 | 54.724 | 76.900 | 1.00 | 42.85 | C |
| ATOM | 794 | CD1 | LEU | F | 105 | −8.654 | 54.127 | 76.107 | 1.00 | 43.00 | C |
| ATOM | 798 | CD2 | LEU | F | 105 | −6.535 | 53.622 | 77.365 | 1.00 | 44.02 | C |
| ATOM | 802 | C | LEU | F | 105 | −8.168 | 57.763 | 76.638 | 1.00 | 39.88 | C |
| ATOM | 803 | O | LEU | F | 105 | −7.499 | 58.360 | 77.473 | 1.00 | 36.24 | O |
| ATOM | 805 | N | LYS | F | 106 | −9.499 | 57.781 | 76.628 | 1.00 | 42.59 | N |
| ATOM | 806 | CA | LYS | F | 106 | −10.289 | 58.513 | 77.623 | 1.00 | 42.33 | C |
| ATOM | 808 | CB | LYS | F | 106 | −10.931 | 59.759 | 76.995 | 1.00 | 43.09 | C |
| ATOM | 811 | CG | LYS | F | 106 | −9.931 | 60.881 | 76.719 | 1.00 | 46.44 | C |
| ATOM | 814 | CD | LYS | F | 106 | −10.416 | 61.893 | 75.690 | 1.00 | 45.30 | C |
| ATOM | 817 | CE | LYS | F | 106 | −11.369 | 62.911 | 76.287 | 1.00 | 48.33 | C |
| ATOM | 820 | NZ | LYS | F | 106 | −12.022 | 63.736 | 75.226 | 1.00 | 46.34 | N |
| ATOM | 824 | C | LYS | F | 106 | −11.372 | 57.607 | 78.190 | 1.00 | 41.92 | C |
| ATOM | 825 | O | LYS | F | 106 | −11.877 | 56.726 | 77.502 | 1.00 | 42.17 | O |
| ATOM | 827 | N | GLY | F | 107 | −11.744 | 57.844 | 79.441 | 1.00 | 42.97 | N |
| ATOM | 828 | CA | GLY | F | 107 | −12.864 | 57.131 | 80.041 | 1.00 | 41.64 | C |
| ATOM | 831 | C | GLY | F | 107 | −12.995 | 57.396 | 81.521 | 1.00 | 40.55 | C |
| ATOM | 832 | O | GLY | F | 107 | −11.992 | 57.536 | 82.223 | 1.00 | 37.90 | O |
| ATOM | 834 | N | TYR | F | 108 | −14.243 | 57.444 | 81.987 | 1.00 | 41.84 | N |
| ATOM | 835 | CA | TYR | F | 108 | −14.570 | 57.697 | 83.392 | 1.00 | 42.31 | C |
| ATOM | 837 | CB | TYR | F | 108 | −15.287 | 59.059 | 83.532 | 1.00 | 49.74 | C |
| ATOM | 840 | CG | TYR | F | 108 | −14.381 | 60.155 | 83.004 | 1.00 | 51.76 | C |
| ATOM | 841 | CD1 | TYR | F | 108 | −14.291 | 60.408 | 81.634 | 1.00 | 52.64 | C |
| ATOM | 843 | CE1 | TYR | F | 108 | −13.411 | 61.360 | 81.121 | 1.00 | 52.72 | C |
| ATOM | 845 | CZ | TYR | F | 108 | −12.591 | 62.071 | 81.979 | 1.00 | 53.84 | C |
| ATOM | 846 | OH | TYR | F | 108 | −11.732 | 63.016 | 81.452 | 1.00 | 53.30 | O |
| ATOM | 848 | CE2 | TYR | F | 108 | −12.647 | 61.839 | 83.352 | 1.00 | 53.56 | C |
| ATOM | 850 | CD2 | TYR | F | 108 | −13.535 | 60.868 | 83.857 | 1.00 | 54.68 | C |
| ATOM | 852 | C | TYR | F | 108 | −15.374 | 56.495 | 83.900 | 1.00 | 38.85 | C |
| ATOM | 853 | O | TYR | F | 108 | −16.499 | 56.270 | 83.475 | 1.00 | 36.85 | O |
| ATOM | 855 | N | PHE | F | 109 | −14.757 | 55.707 | 84.780 | 1.00 | 38.98 | N |
| ATOM | 856 | CA | PHE | F | 109 | −15.309 | 54.424 | 85.226 | 1.00 | 37.99 | C |
| ATOM | 858 | CB | PHE | F | 109 | −14.192 | 53.386 | 85.335 | 1.00 | 37.00 | C |
| ATOM | 861 | CG | PHE | F | 109 | −13.470 | 53.155 | 84.041 | 1.00 | 37.21 | C |
| ATOM | 862 | CD1 | PHE | F | 109 | −12.345 | 53.907 | 83.716 | 1.00 | 36.22 | C |
| ATOM | 864 | CE1 | PHE | F | 109 | −11.690 | 53.714 | 82.505 | 1.00 | 38.76 | C |
| ATOM | 866 | CZ | PHE | F | 109 | −12.158 | 52.756 | 81.603 | 1.00 | 37.56 | C |
| ATOM | 868 | CE2 | PHE | F | 109 | −13.278 | 52.004 | 81.918 | 1.00 | 37.37 | C |
| ATOM | 870 | CD2 | PHE | F | 109 | −13.931 | 52.206 | 83.132 | 1.00 | 37.27 | C |
| ATOM | 872 | C | PHE | F | 109 | −16.016 | 54.585 | 86.554 | 1.00 | 36.21 | C |
| ATOM | 873 | O | PHE | F | 109 | −15.605 | 55.405 | 87.365 | 1.00 | 37.93 | O |
| ATOM | 875 | N | SER | F | 110 | −17.071 | 53.795 | 86.768 | 1.00 | 35.35 | N |
| ATOM | 876 | CA | SER | F | 110 | −17.954 | 53.941 | 87.937 | 1.00 | 36.90 | C |
| ATOM | 878 | CB | SER | F | 110 | −19.313 | 53.286 | 87.670 | 1.00 | 37.72 | C |
| ATOM | 881 | OG | SER | F | 110 | −19.175 | 51.901 | 87.439 | 1.00 | 36.46 | O |
| ATOM | 883 | C | SER | F | 110 | −17.366 | 53.358 | 89.213 | 1.00 | 37.81 | C |
| ATOM | 884 | O | SER | F | 110 | −17.838 | 53.654 | 90.312 | 1.00 | 35.71 | O |
| ATOM | 886 | N | GLN | F | 111 | −16.366 | 52.496 | 89.059 | 1.00 | 37.46 | N |
| ATOM | 887 | CA | GLN | F | 111 | −15.585 | 52.011 | 90.183 | 1.00 | 37.79 | C |
| ATOM | 889 | CB | GLN | F | 111 | −16.162 | 50.700 | 90.726 | 1.00 | 39.00 | C |
| ATOM | 892 | CG | GLN | F | 111 | −16.070 | 49.519 | 89.764 | 1.00 | 39.83 | C |
| ATOM | 895 | CD | GLN | F | 111 | −16.944 | 48.355 | 90.181 | 1.00 | 39.53 | C |
| ATOM | 896 | OE1 | GLN | F | 111 | −17.981 | 48.544 | 90.817 | 1.00 | 42.69 | O |
| ATOM | 897 | NE2 | GLN | F | 111 | −16.532 | 47.142 | 89.820 | 1.00 | 37.36 | N |
| ATOM | 900 | C | GLN | F | 111 | −14.141 | 51.843 | 89.734 | 1.00 | 38.94 | C |
| ATOM | 901 | O | GLN | F | 111 | −13.800 | 52.072 | 88.568 | 1.00 | 38.33 | O |
| ATOM | 903 | N | GLU | F | 112 | −13.294 | 51.457 | 90.675 | 1.00 | 37.98 | N |
| ATOM | 904 | CA | GLU | F | 112 | −11.861 | 51.410 | 90.446 | 1.00 | 39.02 | C |
| ATOM | 906 | CB | GLU | F | 112 | −11.161 | 51.134 | 91.774 | 1.00 | 40.41 | C |
| ATOM | 909 | CG | GLU | F | 112 | −9.764 | 51.648 | 91.876 | 1.00 | 42.75 | C |
| ATOM | 912 | CD | GLU | F | 112 | −9.270 | 51.633 | 93.301 | 1.00 | 43.50 | C |
| ATOM | 913 | OE1 | GLU | F | 112 | −8.564 | 50.670 | 93.664 | 1.00 | 48.34 | O |
| ATOM | 914 | OE2 | GLU | F | 112 | −9.613 | 52.568 | 94.060 | 1.00 | 43.35 | O |
| ATOM | 915 | C | GLU | F | 112 | −11.522 | 50.337 | 89.412 | 1.00 | 38.71 | C |
| ATOM | 916 | O | GLU | F | 112 | −12.122 | 49.262 | 89.416 | 1.00 | 37.43 | O |
| ATOM | 918 | N | VAL | F | 113 | −10.582 | 50.643 | 88.516 | 1.00 | 38.71 | N |
| ATOM | 919 | CA | VAL | F | 113 | −10.156 | 49.694 | 87.485 | 1.00 | 38.20 | C |
| ATOM | 921 | CB | VAL | F | 113 | −10.784 | 49.994 | 86.094 | 1.00 | 37.92 | C |
| ATOM | 923 | CG1 | VAL | F | 113 | −12.295 | 49.971 | 86.161 | 1.00 | 39.45 | C |
| ATOM | 927 | CG2 | VAL | F | 113 | −10.290 | 51.326 | 85.528 | 1.00 | 39.38 | C |
| ATOM | 931 | C | VAL | F | 113 | −8.641 | 49.665 | 87.318 | 1.00 | 38.63 | C |
| ATOM | 932 | O | VAL | F | 113 | −7.935 | 50.583 | 87.740 | 1.00 | 37.29 | O |
| ATOM | 934 | N | ASP | F | 114 | −8.163 | 48.570 | 86.730 | 1.00 | 39.69 | N |
| ATOM | 935 | CA | ASP | F | 114 | −6.838 | 48.504 | 86.123 | 1.00 | 40.13 | C |
| ATOM | 937 | CB | ASP | F | 114 | −6.134 | 47.189 | 86.455 | 1.00 | 39.72 | C |
| ATOM | 940 | CG | ASP | F | 114 | −5.701 | 47.110 | 87.894 | 1.00 | 40.27 | C |
| ATOM | 941 | OD1 | ASP | F | 114 | −5.822 | 48.123 | 88.611 | 1.00 | 42.53 | O |
| ATOM | 942 | OD2 | ASP | F | 114 | −5.245 | 46.029 | 88.314 | 1.00 | 40.84 | O |
| ATOM | 943 | C | ASP | F | 114 | −7.067 | 48.582 | 84.633 | 1.00 | 40.15 | C |
| ATOM | 944 | O | ASP | F | 114 | −7.960 | 47.913 | 84.116 | 1.00 | 38.30 | O |
| ATOM | 946 | N | ILE | F | 115 | −6.291 | 49.414 | 83.945 | 1.00 | 40.64 | N |
| ATOM | 947 | CA | ILE | F | 115 | −6.479 | 49.596 | 82.508 | 1.00 | 41.32 | C |
| ATOM | 949 | CB | ILE | F | 115 | −7.289 | 50.884 | 82.180 | 1.00 | 43.28 | C |
| ATOM | 951 | CG1 | ILE | F | 115 | −7.383 | 51.090 | 80.663 | 1.00 | 42.87 | C |
| ATOM | 954 | CD1 | ILE | F | 115 | −8.538 | 51.958 | 80.253 | 1.00 | 44.46 | C |
| ATOM | 958 | CG2 | ILE | F | 115 | −6.692 | 52.113 | 82.856 | 1.00 | 45.70 | C |
| ATOM | 962 | C | ILE | F | 115 | −5.157 | 49.556 | 81.738 | 1.00 | 40.93 | C |
| ATOM | 963 | O | ILE | F | 115 | −4.181 | 50.205 | 82.111 | 1.00 | 41.08 | O |
| ATOM | 965 | N | SER | F | 116 | −5.143 | 48.778 | 80.662 | 1.00 | 39.89 | N |
| ATOM | 966 | CA | SER | F | 116 | −3.997 | 48.724 | 79.772 | 1.00 | 38.85 | C |
| ATOM | 968 | CB | SER | F | 116 | −3.106 | 47.527 | 80.109 | 1.00 | 36.55 | C |
| ATOM | 971 | OG | SER | F | 116 | −3.864 | 46.411 | 80.529 | 1.00 | 37.81 | O |
| ATOM | 973 | C | SER | F | 116 | −4.408 | 48.712 | 78.295 | 1.00 | 37.29 | C |
| ATOM | 974 | O | SER | F | 116 | −5.486 | 48.246 | 77.935 | 1.00 | 34.01 | O |
| ATOM | 976 | N | LEU | F | 117 | −3.535 | 49.261 | 77.458 | 1.00 | 37.98 | N |
| ATOM | 977 | CA | LEU | F | 117 | −3.716 | 49.258 | 76.014 | 1.00 | 39.62 | C |
| ATOM | 979 | CB | LEU | F | 117 | −3.660 | 50.695 | 75.479 | 1.00 | 41.92 | C |
| ATOM | 982 | CG | LEU | F | 117 | −4.284 | 51.013 | 74.120 | 1.00 | 43.27 | C |
| ATOM | 984 | CD1 | LEU | F | 117 | −5.782 | 50.687 | 74.089 | 1.00 | 41.42 | C |
| ATOM | 988 | CD2 | LEU | F | 117 | −4.039 | 52.488 | 73.789 | 1.00 | 43.71 | C |
| ATOM | 992 | C | LEU | F | 117 | −2.587 | 48.425 | 75.446 | 1.00 | 37.92 | C |
| ATOM | 993 | O | LEU | F | 117 | −1.433 | 48.630 | 75.816 | 1.00 | 38.84 | O |
| ATOM | 995 | N | HIS | F | 118 | −2.929 | 47.466 | 74.581 | 1.00 | 39.17 | N |
| ATOM | 996 | CA | HIS | F | 118 | −1.974 | 46.498 | 74.030 | 1.00 | 37.52 | C |
| ATOM | 998 | CB | HIS | F | 118 | −2.358 | 45.086 | 74.458 | 1.00 | 37.14 | C |
| ATOM | 1001 | CG | HIS | F | 118 | −2.369 | 44.867 | 75.938 | 1.00 | 33.64 | C |
| ATOM | 1002 | ND1 | HIS | F | 118 | −3.399 | 45.290 | 76.747 | 1.00 | 34.43 | N |
| ATOM | 1004 | CE1 | HIS | F | 118 | −3.140 | 44.944 | 77.995 | 1.00 | 34.60 | C |
| ATOM | 1006 | NE2 | HIS | F | 118 | −1.987 | 44.306 | 78.023 | 1.00 | 33.08 | N |
| ATOM | 1008 | CD2 | HIS | F | 118 | −1.485 | 44.242 | 76.748 | 1.00 | 34.88 | C |
| ATOM | 1010 | C | HIS | F | 118 | −1.980 | 46.517 | 72.508 | 1.00 | 37.57 | C |
| ATOM | 1011 | O | HIS | F | 118 | −2.991 | 46.822 | 71.905 | 1.00 | 39.18 | O |
| ATOM | 1013 | N | TYR | F | 119 | −0.854 | 46.157 | 71.901 | 1.00 | 41.91 | N |
| ATOM | 1014 | CA | TYR | F | 119 | −0.730 | 46.035 | 70.437 | 1.00 | 41.70 | C |
| ATOM | 1016 | CB | TYR | F | 119 | 0.334 | 46.999 | 69.902 | 1.00 | 42.07 | C |
| ATOM | 1019 | CG | TYR | F | 119 | 1.752 | 46.676 | 70.331 | 1.00 | 39.49 | C |
| ATOM | 1020 | CD1 | TYR | F | 119 | 2.236 | 47.099 | 71.561 | 1.00 | 38.88 | C |
| ATOM | 1022 | CE1 | TYR | F | 119 | 3.531 | 46.818 | 71.953 | 1.00 | 42.24 | C |
| ATOM | 1024 | CZ | TYR | F | 119 | 4.356 | 46.097 | 71.113 | 1.00 | 42.41 | C |
| ATOM | 1025 | OH | TYR | F | 119 | 5.641 | 45.809 | 71.501 | 1.00 | 43.65 | O |
| ATOM | 1027 | CE2 | TYR | F | 119 | 3.899 | 45.667 | 69.885 | 1.00 | 40.62 | C |
| ATOM | 1029 | CD2 | TYR | F | 119 | 2.604 | 45.965 | 69.499 | 1.00 | 39.35 | C |
| ATOM | 1031 | C | TYR | F | 119 | −0.407 | 44.616 | 69.964 | 1.00 | 41.14 | C |
| ATOM | 1032 | O | TYR | F | 119 | −0.639 | 44.288 | 68.805 | 1.00 | 42.99 | O |
| ATOM | 1034 | N | GLN | F | 120 | 0.164 | 43.799 | 70.845 | 1.00 | 45.37 | N |
| ATOM | 1035 | CA | GLN | F | 120 | 0.428 | 42.384 | 70.565 | 1.00 | 47.68 | C |
| ATOM | 1037 | CB | GLN | F | 120 | 1.875 | 42.174 | 70.091 | 1.00 | 48.74 | C |
| ATOM | 1040 | CG | GLN | F | 120 | 2.049 | 41.196 | 68.896 | 1.00 | 51.02 | C |
| ATOM | 1043 | CD | GLN | F | 120 | 2.153 | 41.914 | 67.553 | 1.00 | 52.54 | C |
| ATOM | 1044 | OE1 | GLN | F | 120 | 1.498 | 41.550 | 66.582 | 1.00 | 47.23 | O |
| ATOM | 1045 | NE2 | GLN | F | 120 | 2.976 | 42.953 | 67.504 | 1.00 | 58.26 | N |
| ATOM | 1048 | C | GLN | F | 120 | 0.172 | 41.608 | 71.856 | 1.00 | 48.98 | C |
| ATOM | 1049 | O | GLN | F | 120 | 0.377 | 42.142 | 72.947 | 1.00 | 48.93 | O |
| ATOM | 1051 | N | LYS | F | 121 | −0.260 | 40.354 | 71.719 | 1.00 | 52.71 | N |
| ATOM | 1052 | CA | LYS | F | 121 | −0.744 | 39.533 | 72.843 | 1.00 | 53.36 | C |
| ATOM | 1054 | CB | LYS | F | 121 | −1.189 | 38.149 | 72.338 | 1.00 | 59.67 | C |
| ATOM | 1057 | CG | LYS | F | 121 | −2.603 | 38.120 | 71.754 | 1.00 | 63.44 | C |
| ATOM | 1060 | CD | LYS | F | 121 | −3.672 | 38.041 | 72.848 | 1.00 | 64.88 | C |
| ATOM | 1063 | CE | LYS | F | 121 | −5.090 | 38.125 | 72.279 | 1.00 | 64.69 | C |
| ATOM | 1066 | NZ | LYS | F | 121 | −5.622 | 39.524 | 72.239 | 1.00 | 63.41 | N |
| ATOM | 1070 | C | LYS | F | 121 | 0.226 | 39.344 | 74.013 | 1.00 | 52.18 | C |
| ATOM | 1071 | O | LYS | F | 121 | −0.175 | 39.461 | 75.173 | 1.00 | 53.77 | O |
| ATOM | 1073 | N | ASP | F | 122 | 1.485 | 39.047 | 73.710 | 1.00 | 50.49 | N |
| ATOM | 1074 | CA | ASP | F | 122 | 2.464 | 38.666 | 74.734 | 1.00 | 51.93 | C |
| ATOM | 1076 | CB | ASP | F | 122 | 3.144 | 37.359 | 74.315 | 1.00 | 54.64 | C |
| ATOM | 1079 | CG | ASP | F | 122 | 2.177 | 36.183 | 74.287 | 1.00 | 57.10 | C |
| ATOM | 1080 | OD1 | ASP | F | 122 | 1.529 | 35.923 | 75.331 | 1.00 | 56.65 | O |
| ATOM | 1081 | OD2 | ASP | F | 122 | 2.069 | 35.525 | 73.224 | 1.00 | 56.29 | O |
| ATOM | 1082 | C | ASP | F | 122 | 3.512 | 39.753 | 74.991 | 1.00 | 49.61 | C |
| ATOM | 1083 | O | ASP | F | 122 | 4.520 | 39.525 | 75.663 | 1.00 | 48.31 | O |
| ATOM | 1085 | N | GLU | F | 123 | 3.240 | 40.943 | 74.475 | 1.00 | 47.41 | N |
| ATOM | 1086 | CA | GLU | F | 123 | 4.182 | 42.045 | 74.485 | 1.00 | 47.94 | C |
| ATOM | 1088 | CB | GLU | F | 123 | 4.167 | 42.707 | 73.097 | 1.00 | 48.69 | C |
| ATOM | 1091 | CG | GLU | F | 123 | 5.534 | 42.988 | 72.481 | 1.00 | 50.68 | C |
| ATOM | 1094 | CD | GLU | F | 123 | 6.401 | 41.761 | 72.274 | 1.00 | 50.52 | C |
| ATOM | 1095 | OE1 | GLU | F | 123 | 5.875 | 40.640 | 72.100 | 1.00 | 55.88 | O |
| ATOM | 1096 | OE2 | GLU | F | 123 | 7.633 | 41.925 | 72.271 | 1.00 | 47.66 | O |
| ATOM | 1097 | C | GLU | F | 123 | 3.760 | 43.026 | 75.586 | 1.00 | 47.13 | C |
| ATOM | 1098 | O | GLU | F | 123 | 2.609 | 43.003 | 76.042 | 1.00 | 46.43 | O |
| ATOM | 1100 | N | GLU | F | 124 | 4.684 | 43.878 | 76.023 | 1.00 | 47.45 | N |
| ATOM | 1101 | CA | GLU | F | 124 | 4.361 | 44.872 | 77.043 | 1.00 | 48.57 | C |
| ATOM | 1103 | CB | GLU | F | 124 | 5.613 | 45.628 | 77.511 | 1.00 | 54.98 | C |
| ATOM | 1106 | CG | GLU | F | 124 | 6.636 | 44.755 | 78.274 | 1.00 | 59.64 | C |
| ATOM | 1109 | CD | GLU | F | 124 | 6.022 | 44.011 | 79.451 | 1.00 | 61.36 | C |
| ATOM | 1110 | OE1 | GLU | F | 124 | 5.454 | 44.669 | 80.352 | 1.00 | 62.77 | O |
| ATOM | 1111 | OE2 | GLU | F | 124 | 6.098 | 42.764 | 79.466 | 1.00 | 61.77 | O |
| ATOM | 1112 | C | GLU | F | 124 | 3.328 | 45.854 | 76.493 | 1.00 | 46.56 | C |
| ATOM | 1113 | O | GLU | F | 124 | 3.401 | 46.231 | 75.327 | 1.00 | 43.72 | O |
| ATOM | 1115 | N | PRO | F | 125 | 2.348 | 46.252 | 77.326 | 1.00 | 43.58 | N |
| ATOM | 1116 | CA | PRO | F | 125 | 1.350 | 47.188 | 76.831 | 1.00 | 44.45 | C |
| ATOM | 1118 | CB | PRO | F | 125 | 0.372 | 47.347 | 78.013 | 1.00 | 45.55 | C |
| ATOM | 1121 | CG | PRO | F | 125 | 1.063 | 46.811 | 79.199 | 1.00 | 45.26 | C |
| ATOM | 1124 | CD | PRO | F | 125 | 2.110 | 45.855 | 78.726 | 1.00 | 43.87 | C |
| ATOM | 1127 | C | PRO | F | 125 | 1.949 | 48.541 | 76.431 | 1.00 | 42.36 | C |
| ATOM | 1128 | O | PRO | F | 125 | 3.025 | 48.918 | 76.901 | 1.00 | 40.37 | O |
| ATOM | 1129 | N | LEU | F | 126 | 1.240 | 49.235 | 75.550 | 1.00 | 42.49 | N |
| ATOM | 1130 | CA | LEU | F | 126 | 1.529 | 50.614 | 75.188 | 1.00 | 45.49 | C |
| ATOM | 1132 | CB | LEU | F | 126 | 0.563 | 51.080 | 74.084 | 1.00 | 42.67 | C |
| ATOM | 1135 | CG | LEU | F | 126 | 0.571 | 50.298 | 72.775 | 1.00 | 42.55 | C |
| ATOM | 1137 | CD1 | LEU | F | 126 | −0.505 | 50.815 | 71.832 | 1.00 | 42.26 | C |
| ATOM | 1141 | CD2 | LEU | F | 126 | 1.951 | 50.366 | 72.134 | 1.00 | 41.34 | C |
| ATOM | 1145 | C | LEU | F | 126 | 1.335 | 51.532 | 76.389 | 1.00 | 46.32 | C |
| ATOM | 1146 | O | LEU | F | 126 | 2.053 | 52.507 | 76.558 | 1.00 | 48.60 | O |
| ATOM | 1148 | N | PHE | F | 127 | 0.336 | 51.200 | 77.200 | 1.00 | 46.43 | N |
| ATOM | 1149 | CA | PHE | F | 127 | −0.155 | 52.052 | 78.265 | 1.00 | 45.87 | C |
| ATOM | 1151 | CB | PHE | F | 127 | −1.286 | 52.924 | 77.726 | 1.00 | 44.94 | C |
| ATOM | 1154 | CG | PHE | F | 127 | −1.910 | 53.816 | 78.758 | 1.00 | 44.97 | C |
| ATOM | 1155 | CD1 | PHE | F | 127 | −1.283 | 54.994 | 79.139 | 1.00 | 45.31 | C |
| ATOM | 1157 | CE1 | PHE | F | 127 | −1.856 | 55.828 | 80.091 | 1.00 | 45.56 | C |
| ATOM | 1159 | CZ | PHE | F | 127 | −3.058 | 55.480 | 80.677 | 1.00 | 45.67 | C |
| ATOM | 1161 | CE2 | PHE | F | 127 | −3.694 | 54.297 | 80.313 | 1.00 | 45.30 | C |
| ATOM | 1163 | CD2 | PHE | F | 127 | −3.120 | 53.477 | 79.352 | 1.00 | 44.56 | C |
| ATOM | 1165 | C | PHE | F | 127 | −0.684 | 51.168 | 79.384 | 1.00 | 45.96 | C |
| ATOM | 1166 | O | PHE | F | 127 | −1.339 | 50.169 | 79.120 | 1.00 | 48.34 | O |
| ATOM | 1168 | N | GLN | F | 128 | −0.418 | 51.550 | 80.630 | 1.00 | 49.16 | N |
| ATOM | 1169 | CA | GLN | F | 128 | −0.879 | 50.780 | 81.778 | 1.00 | 48.72 | C |
| ATOM | 1171 | CB | GLN | F | 128 | 0.161 | 49.710 | 82.103 | 1.00 | 49.23 | C |
| ATOM | 1174 | CG | GLN | F | 128 | −0.332 | 48.626 | 83.031 | 1.00 | 53.05 | C |
| ATOM | 1177 | CD | GLN | F | 128 | 0.641 | 47.463 | 83.141 | 1.00 | 54.97 | C |
| ATOM | 1178 | OE1 | GLN | F | 128 | 1.758 | 47.504 | 82.606 | 1.00 | 55.66 | O |
| ATOM | 1179 | NE2 | GLN | F | 128 | 0.218 | 46.414 | 83.834 | 1.00 | 57.59 | N |
| ATOM | 1182 | C | GLN | F | 128 | −1.136 | 51.670 | 83.004 | 1.00 | 47.48 | C |
| ATOM | 1183 | O | GLN | F | 128 | −0.282 | 52.468 | 83.388 | 1.00 | 48.54 | O |
| ATOM | 1185 | N | LEU | F | 129 | −2.320 | 51.532 | 83.601 | 1.00 | 46.10 | N |
| ATOM | 1186 | CA | LEU | F | 129 | −2.646 | 52.191 | 84.875 | 1.00 | 45.55 | C |
| ATOM | 1188 | CB | LEU | F | 129 | −3.575 | 53.398 | 84.668 | 1.00 | 45.68 | C |
| ATOM | 1191 | CG | LEU | F | 129 | −2.927 | 54.673 | 84.116 | 1.00 | 46.58 | C |
| ATOM | 1193 | CD1 | LEU | F | 129 | −3.965 | 55.760 | 83.896 | 1.00 | 46.33 | C |
| ATOM | 1197 | CD2 | LEU | F | 129 | −1.816 | 55.165 | 85.048 | 1.00 | 47.46 | C |
| ATOM | 1201 | C | LEU | F | 129 | −3.307 | 51.200 | 85.820 | 1.00 | 43.47 | C |
| ATOM | 1202 | O | LEU | F | 129 | −4.074 | 50.336 | 85.383 | 1.00 | 42.34 | O |
| ATOM | 1204 | N | LYS | F | 130 | −3.013 | 51.337 | 87.112 | 1.00 | 40.57 | N |
| ATOM | 1205 | CA | LYS | F | 130 | −3.625 | 50.499 | 88.133 | 1.00 | 43.94 | C |
| ATOM | 1207 | CB | LYS | F | 130 | −2.562 | 49.645 | 88.830 | 1.00 | 47.05 | C |
| ATOM | 1210 | CG | LYS | F | 130 | −1.999 | 48.539 | 87.939 | 1.00 | 49.58 | C |
| ATOM | 1213 | CD | LYS | F | 130 | −0.890 | 47.769 | 88.638 | 1.00 | 50.12 | C |
| ATOM | 1216 | CE | LYS | F | 130 | −0.477 | 46.521 | 87.859 | 1.00 | 51.10 | C |
| ATOM | 1219 | NZ | LYS | F | 130 | 0.240 | 45.545 | 88.751 | 1.00 | 50.89 | N |
| ATOM | 1223 | C | LYS | F | 130 | −4.389 | 51.332 | 89.157 | 1.00 | 42.18 | C |
| ATOM | 1224 | O | LYS | F | 130 | −3.917 | 52.385 | 89.596 | 1.00 | 41.30 | O |
| ATOM | 1226 | N | LYS | F | 131 | −5.576 | 50.847 | 89.520 | 1.00 | 40.68 | N |
| ATOM | 1227 | CA | LYS | F | 131 | −6.397 | 51.439 | 90.574 | 1.00 | 42.56 | C |
| ATOM | 1229 | CB | LYS | F | 131 | −5.719 | 51.276 | 91.942 | 1.00 | 43.61 | C |
| ATOM | 1232 | CG | LYS | F | 131 | −5.399 | 49.836 | 92.285 | 1.00 | 45.79 | C |
| ATOM | 1235 | CD | LYS | F | 131 | −5.033 | 49.681 | 93.750 | 1.00 | 46.38 | C |
| ATOM | 1238 | CE | LYS | F | 131 | −5.089 | 48.229 | 94.196 | 1.00 | 46.61 | C |
| ATOM | 1241 | NZ | LYS | F | 131 | −5.130 | 48.129 | 95.683 | 1.00 | 46.77 | N |
| ATOM | 1245 | C | LYS | F | 131 | −6.749 | 52.904 | 90.305 | 1.00 | 39.02 | C |
| ATOM | 1246 | O | LYS | F | 131 | −6.492 | 53.786 | 91.126 | 1.00 | 38.41 | O |
| ATOM | 1248 | N | VAL | F | 132 | −7.339 | 53.144 | 89.143 | 1.00 | 40.86 | N |
| ATOM | 1249 | CA | VAL | F | 132 | −7.779 | 54.482 | 88.742 | 1.00 | 40.13 | C |
| ATOM | 1251 | CB | VAL | F | 132 | −7.082 | 54.956 | 87.435 | 1.00 | 39.54 | C |
| ATOM | 1253 | CG1 | VAL | F | 132 | −5.594 | 55.190 | 87.668 | 1.00 | 39.44 | C |
| ATOM | 1257 | CG2 | VAL | F | 132 | −7.291 | 53.963 | 86.304 | 1.00 | 40.16 | C |
| ATOM | 1261 | C | VAL | F | 132 | −9.297 | 54.509 | 88.557 | 1.00 | 38.68 | C |
| ATOM | 1262 | O | VAL | F | 132 | −9.935 | 53.473 | 88.348 | 1.00 | 36.99 | O |
| ATOM | 1264 | N | ARG | F | 133 | −9.872 | 55.700 | 88.637 | 1.00 | 39.05 | N |
| ATOM | 1265 | CA | ARG | F | 133 | −11.295 | 55.882 | 88.363 | 1.00 | 38.67 | C |
| ATOM | 1267 | CB | ARG | F | 133 | −11.913 | 56.786 | 89.421 | 1.00 | 34.90 | C |
| ATOM | 1270 | CG | ARG | F | 133 | −12.085 | 56.109 | 90.780 | 1.00 | 33.77 | C |
| ATOM | 1273 | CD | ARG | F | 133 | −13.156 | 55.024 | 90.739 | 1.00 | 35.64 | C |
| ATOM | 1276 | NE | ARG | F | 133 | −14.408 | 55.539 | 90.186 | 1.00 | 37.54 | N |
| ATOM | 1278 | CZ | ARG | F | 133 | −15.308 | 56.252 | 90.863 | 1.00 | 39.09 | C |
| ATOM | 1279 | NH1 | ARG | F | 133 | −15.130 | 56.536 | 92.151 | 1.00 | 39.19 | N |
| ATOM | 1282 | NH2 | ARG | F | 133 | −16.405 | 56.680 | 90.250 | 1.00 | 39.25 | N |
| ATOM | 1285 | C | ARG | F | 133 | −11.525 | 56.441 | 86.959 | 1.00 | 40.17 | C |
| ATOM | 1286 | O | ARG | F | 133 | −12.657 | 56.462 | 86.468 | 1.00 | 41.54 | O |
| ATOM | 1288 | N | SER | F | 134 | −10.449 | 56.883 | 86.314 | 1.00 | 39.98 | N |
| ATOM | 1289 | CA | SER | F | 134 | −10.539 | 57.460 | 84.984 | 1.00 | 41.63 | C |
| ATOM | 1291 | CB | SER | F | 134 | −11.059 | 58.903 | 85.051 | 1.00 | 43.44 | C |
| ATOM | 1294 | OG | SER | F | 134 | −10.106 | 59.781 | 85.621 | 1.00 | 42.63 | O |
| ATOM | 1296 | C | SER | F | 134 | −9.199 | 57.436 | 84.255 | 1.00 | 42.21 | C |
| ATOM | 1297 | O | SER | F | 134 | −8.142 | 57.201 | 84.849 | 1.00 | 39.06 | O |
| ATOM | 1299 | N | VAL | F | 135 | −9.278 | 57.677 | 82.951 | 1.00 | 43.98 | N |
| ATOM | 1300 | CA | VAL | F | 135 | −8.116 | 57.758 | 82.079 | 1.00 | 40.67 | C |
| ATOM | 1302 | CB | VAL | F | 135 | −7.868 | 56.423 | 81.297 | 1.00 | 41.31 | C |
| ATOM | 1304 | CG1 | VAL | F | 135 | −9.018 | 56.068 | 80.390 | 1.00 | 40.29 | C |
| ATOM | 1308 | CG2 | VAL | F | 135 | −6.575 | 56.489 | 80.488 | 1.00 | 45.11 | C |
| ATOM | 1312 | C | VAL | F | 135 | −8.331 | 58.931 | 81.136 | 1.00 | 38.56 | C |
| ATOM | 1313 | O | VAL | F | 135 | −9.449 | 59.180 | 80.691 | 1.00 | 36.27 | O |
| ATOM | 1315 | N | ASN | F | 136 | −7.265 | 59.679 | 80.883 | 1.00 | 41.87 | N |
| ATOM | 1316 | CA | ASN | F | 136 | −7.268 | 60.750 | 79.889 | 1.00 | 41.04 | C |
| ATOM | 1318 | CB | ASN | F | 136 | −7.867 | 62.038 | 80.469 | 1.00 | 43.93 | C |
| ATOM | 1321 | CG | ASN | F | 136 | −7.996 | 63.163 | 79.429 | 1.00 | 43.18 | C |
| ATOM | 1322 | OD1 | ASN | F | 136 | −8.179 | 64.316 | 79.785 | 1.00 | 45.54 | O |
| ATOM | 1323 | ND2 | ASN | F | 136 | −7.904 | 62.822 | 78.157 | 1.00 | 44.48 | N |
| ATOM | 1326 | C | ASN | F | 136 | −5.835 | 60.969 | 79.441 | 1.00 | 41.32 | C |
| ATOM | 1327 | O | ASN | F | 136 | −5.124 | 61.836 | 79.961 | 1.00 | 38.87 | O |
| ATOM | 1329 | N | SER | F | 137 | −5.409 | 60.148 | 78.485 | 1.00 | 43.08 | N |
| ATOM | 1330 | CA | SER | F | 137 | −4.001 | 60.064 | 78.132 | 1.00 | 42.60 | C |
| ATOM | 1332 | CB | SER | F | 137 | −3.387 | 58.785 | 78.718 | 1.00 | 46.32 | C |
| ATOM | 1335 | OG | SER | F | 137 | −3.283 | 58.886 | 80.138 | 1.00 | 48.33 | O |
| ATOM | 1337 | C | SER | F | 137 | −3.781 | 60.130 | 76.626 | 1.00 | 40.64 | C |
| ATOM | 1338 | O | SER | F | 137 | −4.557 | 59.597 | 75.838 | 1.00 | 36.35 | O |
| ATOM | 1340 | N | LEU | F | 138 | −2.708 | 60.814 | 76.254 | 1.00 | 40.13 | N |
| ATOM | 1341 | CA | LEU | F | 138 | −2.285 | 60.944 | 74.880 | 1.00 | 38.99 | C |
| ATOM | 1343 | CB | LEU | F | 138 | −2.327 | 62.424 | 74.494 | 1.00 | 39.95 | C |
| ATOM | 1346 | CG | LEU | F | 138 | −2.316 | 62.793 | 73.010 | 1.00 | 43.27 | C |
| ATOM | 1348 | CD1 | LEU | F | 138 | −3.502 | 62.182 | 72.287 | 1.00 | 43.69 | C |
| ATOM | 1352 | CD2 | LEU | F | 138 | −2.301 | 64.324 | 72.851 | 1.00 | 41.17 | C |
| ATOM | 1356 | C | LEU | F | 138 | −0.865 | 60.389 | 74.805 | 1.00 | 36.23 | C |
| ATOM | 1357 | O | LEU | F | 138 | −0.046 | 60.679 | 75.662 | 1.00 | 34.37 | O |
| ATOM | 1359 | N | MET | F | 139 | −0.575 | 59.569 | 73.801 | 1.00 | 37.81 | N |
| ATOM | 1360 | CA | MET | F | 139 | 0.755 | 58.980 | 73.669 | 1.00 | 36.78 | C |
| ATOM | 1362 | CB | MET | F | 139 | 0.812 | 57.656 | 74.441 | 1.00 | 37.47 | C |
| ATOM | 1365 | CG | MET | F | 139 | 0.100 | 56.496 | 73.740 | 1.00 | 39.18 | C |
| ATOM | 1368 | SD | MET | F | 139 | −0.755 | 55.350 | 74.824 | 1.00 | 42.74 | S |
| ATOM | 1369 | CE | MET | F | 139 | −2.048 | 56.414 | 75.490 | 1.00 | 43.52 | C |
| ATOM | 1373 | C | MET | F | 139 | 1.095 | 58.762 | 72.195 | 1.00 | 35.47 | C |
| ATOM | 1374 | O | MET | F | 139 | 0.226 | 58.820 | 71.341 | 1.00 | 38.62 | O |
| ATOM | 1376 | N | VAL | F | 140 | 2.369 | 58.521 | 71.909 | 1.00 | 36.73 | N |
| ATOM | 1377 | CA | VAL | F | 140 | 2.809 | 58.131 | 70.571 | 1.00 | 36.17 | C |
| ATOM | 1379 | CB | VAL | F | 140 | 3.755 | 59.190 | 69.932 | 1.00 | 32.97 | C |
| ATOM | 1381 | CG1 | VAL | F | 140 | 4.422 | 58.646 | 68.685 | 1.00 | 30.38 | C |
| ATOM | 1385 | CG2 | VAL | F | 140 | 2.973 | 60.453 | 69.600 | 1.00 | 35.43 | C |
| ATOM | 1389 | C | VAL | F | 140 | 3.520 | 56.787 | 70.656 | 1.00 | 37.01 | C |
| ATOM | 1390 | O | VAL | F | 140 | 4.381 | 56.588 | 71.520 | 1.00 | 35.32 | O |
| ATOM | 1392 | N | ALA | F | 141 | 3.160 | 55.884 | 69.744 | 1.00 | 36.58 | N |
| ATOM | 1393 | CA | ALA | F | 141 | 3.733 | 54.547 | 69.678 | 1.00 | 36.38 | C |
| ATOM | 1395 | CB | ALA | F | 141 | 2.747 | 53.540 | 70.243 | 1.00 | 38.09 | C |
| ATOM | 1399 | C | ALA | F | 141 | 4.094 | 54.181 | 68.237 | 1.00 | 36.59 | C |
| ATOM | 1400 | O | ALA | F | 141 | 3.416 | 54.581 | 67.300 | 1.00 | 37.75 | O |
| ATOM | 1402 | N | SER | F | 142 | 5.173 | 53.422 | 68.081 | 1.00 | 36.48 | N |
| ATOM | 1403 | CA | SER | F | 142 | 5.560 | 52.834 | 66.802 | 1.00 | 36.74 | C |
| ATOM | 1405 | CB | SER | F | 142 | 7.075 | 52.623 | 66.753 | 1.00 | 32.96 | C |
| ATOM | 1408 | OG | SER | F | 142 | 7.747 | 53.749 | 67.271 | 1.00 | 39.45 | O |
| ATOM | 1410 | C | SER | F | 142 | 4.874 | 51.484 | 66.607 | 1.00 | 37.19 | C |
| ATOM | 1411 | O | SER | F | 142 | 5.135 | 50.532 | 67.343 | 1.00 | 41.66 | O |
| ATOM | 1413 | N | LEU | F | 143 | 4.014 | 51.402 | 65.604 | 1.00 | 36.82 | N |
| ATOM | 1414 | CA | LEU | F | 143 | 3.284 | 50.173 | 65.306 | 1.00 | 36.42 | C |
| ATOM | 1416 | CB | LEU | F | 143 | 1.786 | 50.401 | 65.506 | 1.00 | 38.11 | C |
| ATOM | 1419 | CG | LEU | F | 143 | 1.369 | 50.986 | 66.870 | 1.00 | 36.43 | C |
| ATOM | 1421 | CD1 | LEU | F | 143 | −0.098 | 51.315 | 66.908 | 1.00 | 38.19 | C |
| ATOM | 1425 | CD2 | LEU | F | 143 | 1.720 | 50.036 | 68.003 | 1.00 | 38.28 | C |
| ATOM | 1429 | C | LEU | F | 143 | 3.589 | 49.718 | 63.876 | 1.00 | 36.46 | C |
| ATOM | 1430 | O | LEU | F | 143 | 4.092 | 50.491 | 63.060 | 1.00 | 38.32 | O |
| ATOM | 1432 | N | THR | F | 144 | 3.320 | 48.447 | 63.601 | 1.00 | 36.76 | N |
| ATOM | 1433 | CA | THR | F | 144 | 3.549 | 47.855 | 62.284 | 1.00 | 36.34 | C |
| ATOM | 1435 | CB | THR | F | 144 | 4.875 | 47.053 | 62.225 | 1.00 | 36.33 | C |
| ATOM | 1437 | OG1 | THR | F | 144 | 4.854 | 45.997 | 63.190 | 1.00 | 36.43 | O |
| ATOM | 1439 | CG2 | THR | F | 144 | 6.069 | 47.950 | 62.491 | 1.00 | 37.74 | C |
| ATOM | 1443 | C | THR | F | 144 | 2.398 | 46.930 | 61.911 | 1.00 | 33.62 | C |
| ATOM | 1444 | O | THR | F | 144 | 1.684 | 46.450 | 62.779 | 1.00 | 30.82 | O |
| ATOM | 1446 | N | TYR | F | 145 | 2.223 | 46.686 | 60.615 | 1.00 | 37.69 | N |
| ATOM | 1447 | CA | TYR | F | 145 | 1.212 | 45.747 | 60.139 | 1.00 | 37.21 | C |
| ATOM | 1449 | CB | TYR | F | 145 | 1.340 | 45.503 | 58.625 | 1.00 | 37.14 | C |
| ATOM | 1452 | CG | TYR | F | 145 | 0.451 | 44.380 | 58.137 | 1.00 | 36.40 | C |
| ATOM | 1453 | CD1 | TYR | F | 145 | −0.930 | 44.537 | 58.087 | 1.00 | 37.63 | C |
| ATOM | 1455 | CE1 | TYR | F | 145 | −1.756 | 43.503 | 57.670 | 1.00 | 36.79 | C |
| ATOM | 1457 | CZ | TYR | F | 145 | −1.203 | 42.299 | 57.304 | 1.00 | 38.34 | C |
| ATOM | 1458 | OH | TYR | F | 145 | −2.016 | 41.277 | 56.877 | 1.00 | 39.10 | O |
| ATOM | 1460 | CE2 | TYR | F | 145 | 0.169 | 42.115 | 57.350 | 1.00 | 37.84 | C |
| ATOM | 1462 | CD2 | TYR | F | 145 | 0.984 | 43.152 | 57.769 | 1.00 | 34.93 | C |
| ATOM | 1464 | C | TYR | F | 145 | 1.351 | 44.436 | 60.909 | 1.00 | 37.43 | C |
| ATOM | 1465 | O | TYR | F | 145 | 2.464 | 43.995 | 61.155 | 1.00 | 39.31 | O |
| ATOM | 1467 | N | LYS | F | 146 | 0.206 | 43.846 | 61.271 | 1.00 | 40.41 | N |
| ATOM | 1468 | CA | LYS | F | 146 | 0.059 | 42.650 | 62.138 | 1.00 | 37.17 | C |
| ATOM | 1470 | CB | LYS | F | 146 | 1.202 | 41.629 | 62.006 | 1.00 | 36.70 | C |
| ATOM | 1473 | CG | LYS | F | 146 | 1.119 | 40.784 | 60.755 | 1.00 | 37.51 | C |
| ATOM | 1476 | CD | LYS | F | 146 | 1.879 | 39.483 | 60.902 | 1.00 | 37.94 | C |
| ATOM | 1479 | CE | LYS | F | 146 | 1.818 | 38.677 | 59.624 | 1.00 | 36.62 | C |
| ATOM | 1482 | NZ | LYS | F | 146 | 2.600 | 37.414 | 59.693 | 1.00 | 35.64 | N |
| ATOM | 1486 | C | LYS | F | 146 | −0.182 | 42.984 | 63.614 | 1.00 | 37.43 | C |
| ATOM | 1487 | O | LYS | F | 146 | −0.666 | 42.136 | 64.367 | 1.00 | 38.06 | O |
| ATOM | 1489 | N | ASP | F | 147 | 0.154 | 44.205 | 64.030 | 1.00 | 37.60 | N |
| ATOM | 1490 | CA | ASP | F | 147 | −0.247 | 44.694 | 65.348 | 1.00 | 36.69 | C |
| ATOM | 1492 | CB | ASP | F | 147 | 0.412 | 46.035 | 65.681 | 1.00 | 35.65 | C |
| ATOM | 1495 | CG | ASP | F | 147 | 1.911 | 45.929 | 65.911 | 1.00 | 34.67 | C |
| ATOM | 1496 | OD1 | ASP | F | 147 | 2.466 | 44.815 | 65.977 | 1.00 | 33.49 | O |
| ATOM | 1497 | OD2 | ASP | F | 147 | 2.540 | 46.993 | 66.040 | 1.00 | 35.12 | O |
| ATOM | 1498 | C | ASP | F | 147 | −1.760 | 44.880 | 65.365 | 1.00 | 38.67 | C |
| ATOM | 1499 | O | ASP | F | 147 | −2.370 | 45.224 | 64.344 | 1.00 | 38.17 | O |
| ATOM | 1501 | N | LYS | F | 148 | −2.356 | 44.642 | 66.529 | 1.00 | 38.74 | N |
| ATOM | 1502 | CA | LYS | F | 148 | −3.790 | 44.832 | 66.739 | 1.00 | 38.13 | C |
| ATOM | 1504 | CB | LYS | F | 148 | −4.547 | 43.502 | 66.641 | 1.00 | 37.69 | C |
| ATOM | 1507 | CG | LYS | F | 148 | −4.341 | 42.742 | 65.330 | 1.00 | 39.03 | C |
| ATOM | 1510 | CD | LYS | F | 148 | −4.873 | 41.323 | 65.431 | 1.00 | 39.83 | C |
| ATOM | 1513 | CE | LYS | F | 148 | −4.769 | 40.593 | 64.124 | 1.00 | 40.49 | C |
| ATOM | 1516 | NZ | LYS | F | 148 | −5.369 | 39.231 | 64.215 | 1.00 | 43.46 | N |
| ATOM | 1520 | C | LYS | F | 148 | −3.969 | 45.447 | 68.121 | 1.00 | 36.95 | C |
| ATOM | 1521 | O | LYS | F | 148 | −3.579 | 44.858 | 69.127 | 1.00 | 35.25 | O |
| ATOM | 1523 | N | VAL | F | 149 | −4.551 | 46.639 | 68.161 | 1.00 | 39.11 | N |
| ATOM | 1524 | CA | VAL | F | 149 | −4.649 | 47.409 | 69.397 | 1.00 | 38.45 | C |
| ATOM | 1526 | CB | VAL | F | 149 | −4.502 | 48.918 | 69.128 | 1.00 | 38.42 | C |
| ATOM | 1528 | CG1 | VAL | F | 149 | −4.780 | 49.738 | 70.376 | 1.00 | 42.66 | C |
| ATOM | 1532 | CG2 | VAL | F | 149 | −3.111 | 49.207 | 68.631 | 1.00 | 39.71 | C |
| ATOM | 1536 | C | VAL | F | 149 | −5.956 | 47.088 | 70.105 | 1.00 | 38.69 | C |
| ATOM | 1537 | O | VAL | F | 149 | −7.040 | 47.227 | 69.543 | 1.00 | 41.58 | O |
| ATOM | 1539 | N | TYR | F | 150 | −5.840 | 46.615 | 71.334 | 1.00 | 37.40 | N |
| ATOM | 1540 | CA | TYR | F | 150 | −7.002 | 46.252 | 72.105 | 1.00 | 38.89 | C |
| ATOM | 1542 | CB | TYR | F | 150 | −7.223 | 44.735 | 72.104 | 1.00 | 35.93 | C |
| ATOM | 1545 | CG | TYR | F | 150 | −6.168 | 43.914 | 72.811 | 1.00 | 36.31 | C |
| ATOM | 1546 | CD1 | TYR | F | 150 | −5.059 | 43.424 | 72.123 | 1.00 | 37.01 | C |
| ATOM | 1548 | CE1 | TYR | F | 150 | −4.092 | 42.659 | 72.770 | 1.00 | 35.19 | C |
| ATOM | 1550 | CZ | TYR | F | 150 | −4.242 | 42.367 | 74.106 | 1.00 | 34.95 | C |
| ATOM | 1551 | OH | TYR | F | 150 | −3.295 | 41.608 | 74.744 | 1.00 | 38.19 | O |
| ATOM | 1553 | CE2 | TYR | F | 150 | −5.339 | 42.834 | 74.808 | 1.00 | 35.82 | C |
| ATOM | 1555 | CD2 | TYR | F | 150 | −6.294 | 43.598 | 74.159 | 1.00 | 35.91 | C |
| ATOM | 1557 | C | TYR | F | 150 | −6.888 | 46.801 | 73.513 | 1.00 | 39.95 | C |
| ATOM | 1558 | O | TYR | F | 150 | −5.802 | 47.163 | 73.975 | 1.00 | 36.11 | O |
| ATOM | 1560 | N | LEU | F | 151 | −8.042 | 46.873 | 74.164 | 1.00 | 41.73 | N |
| ATOM | 1561 | CA | LEU | F | 151 | −8.178 | 47.454 | 75.480 | 1.00 | 42.48 | C |
| ATOM | 1563 | CB | LEU | F | 151 | −9.397 | 48.366 | 75.482 | 1.00 | 43.47 | C |
| ATOM | 1566 | CG | LEU | F | 151 | −9.685 | 49.212 | 76.709 | 1.00 | 44.88 | C |
| ATOM | 1568 | CD1 | LEU | F | 151 | −8.617 | 50.259 | 76.916 | 1.00 | 45.64 | C |
| ATOM | 1572 | CD2 | LEU | F | 151 | −11.046 | 49.844 | 76.526 | 1.00 | 47.81 | C |
| ATOM | 1576 | C | LEU | F | 151 | −8.339 | 46.330 | 76.496 | 1.00 | 42.46 | C |
| ATOM | 1577 | O | LEU | F | 151 | −8.945 | 45.299 | 76.214 | 1.00 | 43.13 | O |
| ATOM | 1579 | N | ASN | F | 152 | −7.790 | 46.543 | 77.680 | 1.00 | 44.63 | N |
| ATOM | 1580 | CA | ASN | F | 152 | −7.792 | 45.551 | 78.741 | 1.00 | 45.55 | C |
| ATOM | 1582 | CB | ASN | F | 152 | −6.417 | 44.872 | 78.772 | 1.00 | 49.23 | C |
| ATOM | 1585 | CG | ASN | F | 152 | −6.305 | 43.747 | 79.791 | 1.00 | 50.43 | C |
| ATOM | 1586 | OD1 | ASN | F | 152 | −7.179 | 43.539 | 80.636 | 1.00 | 53.00 | O |
| ATOM | 1587 | ND2 | ASN | F | 152 | −5.196 | 43.010 | 79.704 | 1.00 | 56.41 | N |
| ATOM | 1590 | C | ASN | F | 152 | −8.086 | 46.305 | 80.032 | 1.00 | 44.78 | C |
| ATOM | 1591 | O | ASN | F | 152 | −7.204 | 46.957 | 80.584 | 1.00 | 43.50 | O |
| ATOM | 1593 | N | VAL | F | 153 | −9.341 | 46.256 | 80.476 | 1.00 | 44.89 | N |
| ATOM | 1594 | CA | VAL | F | 153 | −9.713 | 46.780 | 81.793 | 1.00 | 44.78 | C |
| ATOM | 1596 | CB | VAL | F | 153 | −10.563 | 48.112 | 81.731 | 1.00 | 47.24 | C |
| ATOM | 1598 | CG1 | VAL | F | 153 | −11.030 | 48.431 | 80.319 | 1.00 | 50.93 | C |
| ATOM | 1602 | CG2 | VAL | F | 153 | −11.723 | 48.123 | 82.726 | 1.00 | 44.92 | C |
| ATOM | 1606 | C | VAL | F | 153 | −10.329 | 45.696 | 82.693 | 1.00 | 44.28 | C |
| ATOM | 1607 | O | VAL | F | 153 | −11.247 | 44.969 | 82.303 | 1.00 | 44.32 | O |
| ATOM | 1609 | N | THR | F | 154 | −9.769 | 45.581 | 83.892 | 1.00 | 39.88 | N |
| ATOM | 1610 | CA | THR | F | 154 | −10.224 | 44.631 | 84.880 | 1.00 | 42.98 | C |
| ATOM | 1612 | CB | THR | F | 154 | −9.121 | 43.605 | 85.219 | 1.00 | 45.60 | C |
| ATOM | 1614 | OG1 | THR | F | 154 | −7.945 | 44.290 | 85.665 | 1.00 | 46.84 | O |
| ATOM | 1616 | CG2 | THR | F | 154 | −8.779 | 42.767 | 83.992 | 1.00 | 45.83 | C |
| ATOM | 1620 | C | THR | F | 154 | −10.649 | 45.385 | 86.137 | 1.00 | 40.52 | C |
| ATOM | 1621 | O | THR | F | 154 | −10.124 | 46.460 | 86.449 | 1.00 | 35.32 | O |
| ATOM | 1623 | N | THR | F | 155 | −11.612 | 44.812 | 86.847 | 1.00 | 39.29 | N |
| ATOM | 1624 | CA | THR | F | 155 | −12.223 | 45.485 | 87.980 | 1.00 | 39.79 | C |
| ATOM | 1626 | CB | THR | F | 155 | −13.408 | 46.378 | 87.513 | 1.00 | 38.52 | C |
| ATOM | 1628 | OG1 | THR | F | 155 | −13.757 | 47.310 | 88.542 | 1.00 | 39.43 | O |
| ATOM | 1630 | CG2 | THR | F | 155 | −14.631 | 45.544 | 87.130 | 1.00 | 38.27 | C |
| ATOM | 1634 | C | THR | F | 155 | −12.676 | 44.445 | 88.987 | 1.00 | 39.31 | C |
| ATOM | 1635 | O | THR | F | 155 | −12.491 | 43.246 | 88.776 | 1.00 | 38.45 | O |
| ATOM | 1637 | N | ASP | F | 156 | −13.250 | 44.916 | 90.087 | 1.00 | 42.41 | N |
| ATOM | 1638 | CA | ASP | F | 156 | −13.821 | 44.043 | 91.103 | 1.00 | 41.26 | C |
| ATOM | 1640 | CB | ASP | F | 156 | −14.253 | 44.887 | 92.307 | 1.00 | 43.55 | C |
| ATOM | 1643 | CG | ASP | F | 156 | −14.673 | 44.060 | 93.504 | 1.00 | 43.54 | C |
| ATOM | 1644 | OD1 | ASP | F | 156 | −14.681 | 42.814 | 93.430 | 1.00 | 45.44 | O |
| ATOM | 1645 | OD2 | ASP | F | 156 | −14.997 | 44.681 | 94.536 | 1.00 | 44.53 | O |
| ATOM | 1646 | C | ASP | F | 156 | −15.009 | 43.317 | 90.476 | 1.00 | 40.85 | C |
| ATOM | 1647 | O | ASP | F | 156 | −16.057 | 43.918 | 90.246 | 1.00 | 39.23 | O |
| ATOM | 1649 | N | ASN | F | 157 | −14.827 | 42.029 | 90.182 | 1.00 | 42.16 | N |
| ATOM | 1650 | CA | ASN | F | 157 | −15.862 | 41.213 | 89.532 | 1.00 | 42.08 | C |
| ATOM | 1652 | CB | ASN | F | 157 | −15.254 | 39.916 | 88.978 | 1.00 | 42.43 | C |
| ATOM | 1655 | CG | ASN | F | 157 | −14.400 | 40.148 | 87.735 | 1.00 | 42.91 | C |
| ATOM | 1656 | OD1 | ASN | F | 157 | −14.785 | 40.886 | 86.826 | 1.00 | 41.58 | O |
| ATOM | 1657 | ND2 | ASN | F | 157 | −13.244 | 39.496 | 87.685 | 1.00 | 42.16 | N |
| ATOM | 1660 | C | ASN | F | 157 | −17.053 | 40.885 | 90.438 | 1.00 | 40.86 | C |
| ATOM | 1661 | O | ASN | F | 157 | −18.027 | 40.276 | 89.986 | 1.00 | 37.98 | O |
| ATOM | 1663 | N | THR | F | 158 | −16.964 | 41.277 | 91.709 | 1.00 | 43.69 | N |
| ATOM | 1664 | CA | THR | F | 158 | −18.089 | 41.193 | 92.644 | 1.00 | 43.92 | C |
| ATOM | 1666 | CB | THR | F | 158 | −17.623 | 41.457 | 94.094 | 1.00 | 43.82 | C |
| ATOM | 1668 | OG1 | THR | F | 158 | −16.826 | 40.354 | 94.541 | 1.00 | 43.15 | O |
| ATOM | 1670 | CG2 | THR | F | 158 | −18.805 | 41.635 | 95.043 | 1.00 | 45.34 | C |
| ATOM | 1674 | C | THR | F | 158 | −19.193 | 42.185 | 92.277 | 1.00 | 45.75 | C |
| ATOM | 1675 | O | THR | F | 158 | −20.370 | 41.824 | 92.270 | 1.00 | 46.49 | O |
| ATOM | 1677 | N | SER | F | 159 | −18.811 | 43.430 | 91.986 | 1.00 | 46.33 | N |
| ATOM | 1678 | CA | SER | F | 159 | −19.776 | 44.466 | 91.613 | 1.00 | 45.13 | C |
| ATOM | 1680 | CB | SER | F | 159 | −19.561 | 45.742 | 92.433 | 1.00 | 46.86 | C |
| ATOM | 1683 | OG | SER | F | 159 | −18.232 | 46.204 | 92.326 | 1.00 | 48.59 | O |
| ATOM | 1685 | C | SER | F | 159 | −19.713 | 44.770 | 90.119 | 1.00 | 43.03 | C |
| ATOM | 1686 | O | SER | F | 159 | −18.957 | 45.631 | 89.675 | 1.00 | 43.47 | O |
| ATOM | 1688 | N | LEU | F | 160 | −20.515 | 44.038 | 89.354 | 1.00 | 42.68 | N |
| ATOM | 1689 | CA | LEU | F | 160 | −20.684 | 44.283 | 87.929 | 1.00 | 42.18 | C |
| ATOM | 1691 | CB | LEU | F | 160 | −20.339 | 43.012 | 87.145 | 1.00 | 41.14 | C |
| ATOM | 1694 | CG | LEU | F | 160 | −18.884 | 42.539 | 87.262 | 1.00 | 41.56 | C |
| ATOM | 1696 | CD1 | LEU | F | 160 | −18.774 | 41.078 | 86.874 | 1.00 | 43.58 | C |
| ATOM | 1700 | CD2 | LEU | F | 160 | −17.935 | 43.389 | 86.416 | 1.00 | 42.66 | C |
| ATOM | 1704 | C | LEU | F | 160 | −22.109 | 44.746 | 87.592 | 1.00 | 41.83 | C |
| ATOM | 1705 | O | LEU | F | 160 | −22.399 | 45.043 | 86.432 | 1.00 | 42.81 | O |
| ATOM | 1707 | N | ASP | F | 161 | −22.983 | 44.832 | 88.597 | 1.00 | 40.24 | N |
| ATOM | 1708 | CA | ASP | F | 161 | −24.390 | 45.183 | 88.377 | 1.00 | 42.77 | C |
| ATOM | 1710 | CB | ASP | F | 161 | −25.193 | 45.077 | 89.687 | 1.00 | 46.10 | C |
| ATOM | 1713 | CG | ASP | F | 161 | −25.356 | 43.641 | 90.161 | 1.00 | 50.56 | C |
| ATOM | 1714 | OD1 | ASP | F | 161 | −24.516 | 42.787 | 89.791 | 1.00 | 52.09 | O |
| ATOM | 1715 | OD2 | ASP | F | 161 | −26.325 | 43.363 | 90.905 | 1.00 | 53.72 | O |
| ATOM | 1716 | C | ASP | F | 161 | −24.573 | 46.577 | 87.767 | 1.00 | 42.75 | C |
| ATOM | 1717 | O | ASP | F | 161 | −25.483 | 46.787 | 86.963 | 1.00 | 42.99 | O |
| ATOM | 1719 | N | ASP | F | 162 | −23.721 | 47.525 | 88.154 | 1.00 | 41.00 | N |
| ATOM | 1720 | CA | ASP | F | 162 | −23.784 | 48.884 | 87.613 | 1.00 | 40.35 | C |
| ATOM | 1722 | CB | ASP | F | 162 | −24.499 | 49.812 | 88.605 | 1.00 | 40.18 | C |
| ATOM | 1725 | CG | ASP | F | 162 | −24.884 | 51.150 | 87.991 | 1.00 | 41.36 | C |
| ATOM | 1726 | OD1 | ASP | F | 162 | −25.686 | 51.170 | 87.029 | 1.00 | 39.61 | O |
| ATOM | 1727 | OD2 | ASP | F | 162 | −24.385 | 52.185 | 88.479 | 1.00 | 41.96 | O |
| ATOM | 1728 | C | ASP | F | 162 | −22.378 | 49.394 | 87.279 | 1.00 | 40.42 | C |
| ATOM | 1729 | O | ASP | F | 162 | −22.045 | 50.557 | 87.526 | 1.00 | 43.19 | O |
| ATOM | 1731 | N | PHE | F | 163 | −21.556 | 48.508 | 86.719 | 1.00 | 37.13 | N |
| ATOM | 1732 | CA | PHE | F | 163 | −20.229 | 48.878 | 86.254 | 1.00 | 36.99 | C |
| ATOM | 1734 | CB | PHE | F | 163 | −19.283 | 47.669 | 86.224 | 1.00 | 34.56 | C |
| ATOM | 1737 | CG | PHE | F | 163 | −17.904 | 47.997 | 85.704 | 1.00 | 33.92 | C |
| ATOM | 1738 | CD1 | PHE | F | 163 | −17.172 | 49.047 | 86.252 | 1.00 | 32.93 | C |
| ATOM | 1740 | CE1 | PHE | F | 163 | −15.905 | 49.364 | 85.767 | 1.00 | 34.65 | C |
| ATOM | 1742 | CZ | PHE | F | 163 | −15.354 | 48.616 | 84.735 | 1.00 | 34.07 | C |
| ATOM | 1744 | CE2 | PHE | F | 163 | −16.073 | 47.569 | 84.183 | 1.00 | 32.90 | C |
| ATOM | 1746 | CD2 | PHE | F | 163 | −17.339 | 47.262 | 84.667 | 1.00 | 33.44 | C |
| ATOM | 1748 | C | PHE | F | 163 | −20.342 | 49.483 | 84.862 | 1.00 | 36.95 | C |
| ATOM | 1749 | O | PHE | F | 163 | −20.815 | 48.832 | 83.933 | 1.00 | 36.94 | O |
| ATOM | 1751 | N | HIS | F | 164 | −19.906 | 50.730 | 84.718 | 1.00 | 37.09 | N |
| ATOM | 1752 | CA | HIS | F | 164 | −20.038 | 51.435 | 83.450 | 1.00 | 35.77 | C |
| ATOM | 1754 | CB | HIS | F | 164 | −21.398 | 52.147 | 83.361 | 1.00 | 32.87 | C |
| ATOM | 1757 | CG | HIS | F | 164 | −22.555 | 51.212 | 83.173 | 1.00 | 30.54 | C |
| ATOM | 1758 | ND1 | HIS | F | 164 | −22.752 | 50.495 | 82.013 | 1.00 | 29.35 | N |
| ATOM | 1760 | CE1 | HIS | F | 164 | −23.833 | 49.747 | 82.137 | 1.00 | 28.11 | C |
| ATOM | 1762 | NE2 | HIS | F | 164 | −24.342 | 49.947 | 83.338 | 1.00 | 26.02 | N |
| ATOM | 1764 | CD2 | HIS | F | 164 | −23.558 | 50.854 | 84.010 | 1.00 | 28.88 | C |
| ATOM | 1766 | C | HIS | F | 164 | −18.915 | 52.430 | 83.246 | 1.00 | 37.17 | C |
| ATOM | 1767 | O | HIS | F | 164 | −18.243 | 52.840 | 84.189 | 1.00 | 35.87 | O |
| ATOM | 1769 | N | VAL | F | 165 | −18.723 | 52.799 | 81.987 | 1.00 | 38.34 | N |
| ATOM | 1770 | CA | VAL | F | 165 | −17.775 | 53.829 | 81.609 | 1.00 | 36.95 | C |
| ATOM | 1772 | CB | VAL | F | 165 | −16.621 | 53.260 | 80.730 | 1.00 | 36.32 | C |
| ATOM | 1774 | CG1 | VAL | F | 165 | −17.134 | 52.759 | 79.379 | 1.00 | 38.21 | C |
| ATOM | 1778 | CG2 | VAL | F | 165 | −15.526 | 54.301 | 80.532 | 1.00 | 36.28 | C |
| ATOM | 1782 | C | VAL | F | 165 | −18.536 | 54.943 | 80.885 | 1.00 | 38.26 | C |
| ATOM | 1783 | O | VAL | F | 165 | −19.403 | 54.676 | 80.036 | 1.00 | 38.14 | O |
| ATOM | 1785 | N | ASN | F | 166 | −18.238 | 56.186 | 81.260 | 1.00 | 35.73 | N |
| ATOM | 1786 | CA | ASN | F | 166 | −18.718 | 57.348 | 80.533 | 1.00 | 35.61 | C |
| ATOM | 1788 | CB | ASN | F | 166 | −19.349 | 58.362 | 81.481 | 1.00 | 33.13 | C |
| ATOM | 1791 | CG | ASN | F | 166 | −20.704 | 57.934 | 81.985 | 1.00 | 31.58 | C |
| ATOM | 1792 | OD1 | ASN | F | 166 | −21.551 | 57.464 | 81.223 | 1.00 | 27.56 | O |
| ATOM | 1793 | ND2 | ASN | F | 166 | −20.930 | 58.120 | 83.284 | 1.00 | 31.50 | N |
| ATOM | 1796 | C | ASN | F | 166 | −17.565 | 58.007 | 79.780 | 1.00 | 34.95 | C |
| ATOM | 1797 | O | ASN | F | 166 | −16.458 | 58.128 | 80.303 | 1.00 | 36.96 | O |
| ATOM | 1799 | N | GLY | F | 167 | −17.834 | 58.434 | 78.552 | 1.00 | 34.63 | N |
| ATOM | 1800 | CA | GLY | F | 167 | −16.824 | 59.098 | 77.727 | 1.00 | 35.89 | C |
| ATOM | 1803 | C | GLY | F | 167 | −15.706 | 58.170 | 77.284 | 1.00 | 35.95 | C |
| ATOM | 1804 | O | GLY | F | 167 | −14.596 | 58.626 | 77.007 | 1.00 | 41.18 | O |
| ATOM | 1806 | N | GLY | F | 168 | −16.003 | 56.872 | 77.223 | 1.00 | 34.60 | N |
| ATOM | 1807 | CA | GLY | F | 168 | −15.056 | 55.859 | 76.777 | 1.00 | 36.43 | C |
| ATOM | 1810 | C | GLY | F | 168 | −14.712 | 56.046 | 75.312 | 1.00 | 37.81 | C |
| ATOM | 1811 | O | GLY | F | 168 | −15.562 | 55.877 | 74.445 | 1.00 | 39.12 | O |
| ATOM | 1813 | N | GLU | F | 169 | −13.456 | 56.389 | 75.042 | 1.00 | 40.78 | N |
| ATOM | 1814 | CA | GLU | F | 169 | −13.035 | 56.812 | 73.715 | 1.00 | 38.12 | C |
| ATOM | 1816 | CB | GLU | F | 169 | −13.228 | 58.315 | 73.584 | 1.00 | 41.40 | C |
| ATOM | 1819 | CG | GLU | F | 169 | −12.997 | 58.888 | 72.190 | 1.00 | 41.79 | C |
| ATOM | 1822 | CD | GLU | F | 169 | −13.021 | 60.393 | 72.219 | 1.00 | 42.87 | C |
| ATOM | 1823 | OE1 | GLU | F | 169 | −11.976 | 61.005 | 72.540 | 1.00 | 40.82 | O |
| ATOM | 1824 | OE2 | GLU | F | 169 | −14.095 | 60.961 | 71.944 | 1.00 | 46.26 | O |
| ATOM | 1825 | C | GLU | F | 169 | −11.579 | 56.465 | 73.457 | 1.00 | 36.78 | C |
| ATOM | 1826 | O | GLU | F | 169 | −10.705 | 56.803 | 74.259 | 1.00 | 36.62 | O |
| ATOM | 1828 | N | LEU | F | 170 | −11.341 | 55.779 | 72.337 | 1.00 | 35.81 | N |
| ATOM | 1829 | CA | LEU | F | 170 | −10.003 | 55.439 | 71.876 | 1.00 | 35.89 | C |
| ATOM | 1831 | CB | LEU | F | 170 | −9.784 | 53.925 | 71.935 | 1.00 | 37.28 | C |
| ATOM | 1834 | CG | LEU | F | 170 | −8.571 | 53.317 | 71.213 | 1.00 | 36.19 | C |
| ATOM | 1836 | CD1 | LEU | F | 170 | −7.275 | 53.769 | 71.847 | 1.00 | 36.63 | C |
| ATOM | 1840 | CD2 | LEU | F | 170 | −8.662 | 51.791 | 71.212 | 1.00 | 36.34 | C |
| ATOM | 1844 | C | LEU | F | 170 | −9.822 | 55.954 | 70.445 | 1.00 | 37.75 | C |
| ATOM | 1845 | O | LEU | F | 170 | −10.513 | 55.510 | 69.514 | 1.00 | 34.03 | O |
| ATOM | 1847 | N | ILE | F | 171 | −8.901 | 56.905 | 70.283 | 1.00 | 36.62 | N |
| ATOM | 1848 | CA | ILE | F | 171 | −8.543 | 57.421 | 68.971 | 1.00 | 37.41 | C |
| ATOM | 1850 | CB | ILE | F | 171 | −8.624 | 58.960 | 68.946 | 1.00 | 35.58 | C |
| ATOM | 1852 | CG1 | ILE | F | 171 | −10.070 | 59.424 | 69.136 | 1.00 | 35.28 | C |
| ATOM | 1855 | CD1 | ILE | F | 171 | −10.200 | 60.938 | 69.389 | 1.00 | 35.66 | C |
| ATOM | 1859 | CG2 | ILE | F | 171 | −8.030 | 59.525 | 67.639 | 1.00 | 36.02 | C |
| ATOM | 1863 | C | ILE | F | 171 | −7.124 | 56.961 | 68.597 | 1.00 | 36.06 | C |
| ATOM | 1864 | O | ILE | F | 171 | −6.227 | 56.987 | 69.434 | 1.00 | 38.72 | O |
| ATOM | 1866 | N | LEU | F | 172 | −6.939 | 56.507 | 67.357 | 1.00 | 36.84 | N |
| ATOM | 1867 | CA | LEU | F | 172 | −5.587 | 56.336 | 66.788 | 1.00 | 37.28 | C |
| ATOM | 1869 | CB | LEU | F | 172 | −5.221 | 54.879 | 66.506 | 1.00 | 39.72 | C |
| ATOM | 1872 | CG | LEU | F | 172 | −5.737 | 53.585 | 67.146 | 1.00 | 41.24 | C |
| ATOM | 1874 | CD1 | LEU | F | 172 | −4.560 | 52.624 | 67.209 | 1.00 | 42.11 | C |
| ATOM | 1878 | CD2 | LEU | F | 172 | −6.362 | 53.698 | 68.492 | 1.00 | 44.22 | C |
| ATOM | 1882 | C | LEU | F | 172 | −5.440 | 57.134 | 65.485 | 1.00 | 37.58 | C |
| ATOM | 1883 | O | LEU | F | 172 | −6.347 | 57.145 | 64.641 | 1.00 | 35.52 | O |
| ATOM | 1885 | N | ILE | F | 173 | −4.302 | 57.808 | 65.331 | 1.00 | 37.82 | N |
| ATOM | 1886 | CA | ILE | F | 173 | −4.026 | 58.607 | 64.131 | 1.00 | 36.23 | C |
| ATOM | 1888 | CB | ILE | F | 173 | −4.186 | 60.120 | 64.388 | 1.00 | 36.94 | C |
| ATOM | 1890 | CG1 | ILE | F | 173 | −5.550 | 60.438 | 65.020 | 1.00 | 37.84 | C |
| ATOM | 1893 | CD1 | ILE | F | 173 | −5.674 | 61.864 | 65.563 | 1.00 | 36.14 | C |
| ATOM | 1897 | CG2 | ILE | F | 173 | −4.006 | 60.898 | 63.099 | 1.00 | 34.14 | C |
| ATOM | 1901 | C | ILE | F | 173 | −2.608 | 58.346 | 63.629 | 1.00 | 36.89 | C |
| ATOM | 1902 | O | ILE | F | 173 | −1.628 | 58.716 | 64.283 | 1.00 | 35.93 | O |
| ATOM | 1904 | N | HIS | F | 174 | −2.516 | 57.702 | 62.465 | 1.00 | 38.49 | N |
| ATOM | 1905 | CA | HIS | F | 174 | −1.249 | 57.450 | 61.779 | 1.00 | 37.86 | C |
| ATOM | 1907 | CB | HIS | F | 174 | −1.535 | 56.725 | 60.462 | 1.00 | 39.54 | C |
| ATOM | 1910 | CG | HIS | F | 174 | −0.330 | 56.154 | 59.783 | 1.00 | 39.08 | C |
| ATOM | 1911 | ND1 | HIS | F | 174 | −0.203 | 56.120 | 58.409 | 1.00 | 39.03 | N |
| ATOM | 1913 | CE1 | HIS | F | 174 | 0.937 | 55.536 | 58.088 | 1.00 | 37.60 | C |
| ATOM | 1915 | NE2 | HIS | F | 174 | 1.555 | 55.192 | 59.204 | 1.00 | 37.87 | N |
| ATOM | 1917 | CD2 | HIS | F | 174 | 0.781 | 55.561 | 60.278 | 1.00 | 36.83 | C |
| ATOM | 1919 | C | HIS | F | 174 | −0.531 | 58.767 | 61.516 | 1.00 | 37.68 | C |
| ATOM | 1920 | O | HIS | F | 174 | −1.162 | 59.757 | 61.129 | 1.00 | 35.00 | O |
| ATOM | 1922 | N | GLN | F | 175 | 0.783 | 58.783 | 61.740 | 1.00 | 37.16 | N |
| ATOM | 1923 | CA | GLN | F | 175 | 1.583 | 59.997 | 61.547 | 1.00 | 37.10 | C |
| ATOM | 1925 | CB | GLN | F | 175 | 2.348 | 60.347 | 62.831 | 1.00 | 38.15 | C |
| ATOM | 1928 | CG | GLN | F | 175 | 1.469 | 60.622 | 64.042 | 1.00 | 37.55 | C |
| ATOM | 1931 | CD | GLN | F | 175 | 0.620 | 61.860 | 63.880 | 1.00 | 36.83 | C |
| ATOM | 1932 | OE1 | GLN | F | 175 | 1.133 | 62.945 | 63.572 | 1.00 | 37.36 | O |
| ATOM | 1933 | NE2 | GLN | F | 175 | −0.682 | 61.716 | 64.098 | 1.00 | 31.75 | N |
| ATOM | 1936 | C | GLN | F | 175 | 2.565 | 59.911 | 60.379 | 1.00 | 35.45 | C |
| ATOM | 1937 | O | GLN | F | 175 | 3.261 | 60.884 | 60.086 | 1.00 | 34.80 | O |
| ATOM | 1939 | N | ASN | F | 176 | 2.592 | 58.773 | 59.692 | 1.00 | 39.05 | N |
| ATOM | 1940 | CA | ASN | F | 176 | 3.566 | 58.528 | 58.629 | 1.00 | 37.50 | C |
| ATOM | 1942 | CB | ASN | F | 176 | 4.561 | 57.468 | 59.086 | 1.00 | 33.92 | C |
| ATOM | 1945 | CG | ASN | F | 176 | 5.372 | 57.922 | 60.246 | 1.00 | 32.87 | C |
| ATOM | 1946 | OD1 | ASN | F | 176 | 4.998 | 57.691 | 61.387 | 1.00 | 33.38 | O |
| ATOM | 1947 | ND2 | ASN | F | 176 | 6.477 | 58.618 | 59.970 | 1.00 | 32.08 | N |
| ATOM | 1950 | C | ASN | F | 176 | 2.956 | 58.090 | 57.314 | 1.00 | 36.45 | C |
| ATOM | 1951 | O | ASN | F | 176 | 3.328 | 57.043 | 56.796 | 1.00 | 33.58 | O |
| ATOM | 1953 | N | PRO | F | 177 | 2.049 | 58.906 | 56.744 | 1.00 | 39.03 | N |
| ATOM | 1954 | CA | PRO | F | 177 | 1.533 | 58.596 | 55.410 | 1.00 | 39.64 | C |
| ATOM | 1956 | CB | PRO | F | 177 | 0.744 | 59.865 | 55.020 | 1.00 | 40.97 | C |
| ATOM | 1959 | CG | PRO | F | 177 | 1.093 | 60.907 | 56.034 | 1.00 | 39.58 | C |
| ATOM | 1962 | CD | PRO | F | 177 | 1.469 | 60.151 | 57.277 | 1.00 | 40.02 | C |
| ATOM | 1965 | C | PRO | F | 177 | 2.655 | 58.327 | 54.402 | 1.00 | 39.95 | C |
| ATOM | 1966 | O | PRO | F | 177 | 3.664 | 59.034 | 54.399 | 1.00 | 43.14 | O |
| ATOM | 1967 | N | GLY | F | 178 | 2.473 | 57.306 | 53.572 | 1.00 | 40.05 | N |
| ATOM | 1968 | CA | GLY | F | 178 | 3.455 | 56.923 | 52.559 | 1.00 | 43.05 | C |
| ATOM | 1971 | C | GLY | F | 178 | 4.278 | 55.699 | 52.937 | 1.00 | 44.93 | C |
| ATOM | 1972 | O | GLY | F | 178 | 5.001 | 55.143 | 52.101 | 1.00 | 43.29 | O |
| ATOM | 1974 | N | GLU | F | 179 | 4.168 | 55.279 | 54.196 | 1.00 | 44.71 | N |
| ATOM | 1975 | CA | GLU | F | 179 | 4.911 | 54.127 | 54.701 | 1.00 | 44.56 | C |
| ATOM | 1977 | CB | GLU | F | 179 | 5.015 | 54.196 | 56.236 | 1.00 | 50.41 | C |
| ATOM | 1980 | CG | GLU | F | 179 | 6.416 | 53.983 | 56.791 | 1.00 | 59.02 | C |
| ATOM | 1983 | CD | GLU | F | 179 | 7.362 | 55.145 | 56.496 | 1.00 | 63.76 | C |
| ATOM | 1984 | OE1 | GLU | F | 179 | 7.121 | 56.260 | 57.000 | 1.00 | 70.53 | O |
| ATOM | 1985 | OE2 | GLU | F | 179 | 8.359 | 54.943 | 55.770 | 1.00 | 64.40 | O |
| ATOM | 1986 | C | GLU | F | 179 | 4.213 | 52.838 | 54.236 | 1.00 | 42.08 | C |
| ATOM | 1987 | O | GLU | F | 179 | 3.153 | 52.891 | 53.614 | 1.00 | 43.52 | O |
| ATOM | 1989 | N | PHE | F | 180 | 4.810 | 51.686 | 54.523 | 1.00 | 40.75 | N |
| ATOM | 1990 | CA | PHE | F | 180 | 4.231 | 50.397 | 54.128 | 1.00 | 39.16 | C |
| ATOM | 1992 | CB | PHE | F | 180 | 5.093 | 49.238 | 54.631 | 1.00 | 37.33 | C |
| ATOM | 1995 | CG | PHE | F | 180 | 4.485 | 47.882 | 54.372 | 1.00 | 38.10 | C |
| ATOM | 1996 | CD1 | PHE | F | 180 | 4.597 | 47.286 | 53.121 | 1.00 | 35.59 | C |
| ATOM | 1998 | CE1 | PHE | F | 180 | 4.025 | 46.047 | 52.876 | 1.00 | 36.56 | C |
| ATOM | 2000 | CZ | PHE | F | 180 | 3.325 | 45.394 | 53.881 | 1.00 | 35.30 | C |
| ATOM | 2002 | CE2 | PHE | F | 180 | 3.196 | 45.980 | 55.123 | 1.00 | 34.78 | C |
| ATOM | 2004 | CD2 | PHE | F | 180 | 3.776 | 47.217 | 55.368 | 1.00 | 34.91 | C |
| ATOM | 2006 | C | PHE | F | 180 | 2.811 | 50.201 | 54.661 | 1.00 | 39.57 | C |
| ATOM | 2007 | O | PHE | F | 180 | 2.558 | 50.418 | 55.849 | 1.00 | 39.17 | O |
| ATOM | 2009 | N | CYS | F | 181 | 1.900 | 49.788 | 53.776 | 1.00 | 36.56 | N |
| ATOM | 2010 | CA | CYS | F | 181 | 0.534 | 49.443 | 54.152 | 1.00 | 39.15 | C |
| ATOM | 2012 | CB | CYS | F | 181 | −0.468 | 50.493 | 53.654 | 1.00 | 40.67 | C |
| ATOM | 2015 | SG | CYS | F | 181 | −0.275 | 52.133 | 54.349 | 1.00 | 42.33 | S |
| ATOM | 2017 | C | CYS | F | 181 | 0.134 | 48.104 | 53.569 | 1.00 | 38.47 | C |
| ATOM | 2018 | O | CYS | F | 181 | 0.686 | 47.674 | 52.561 | 1.00 | 39.25 | O |
| ATOM | 2020 | N | VAL | F | 182 | −0.822 | 47.446 | 54.219 | 1.00 | 39.80 | N |
| ATOM | 2021 | CA | VAL | F | 182 | −1.576 | 46.361 | 53.594 | 1.00 | 42.69 | C |
| ATOM | 2023 | CB | VAL | F | 182 | −1.490 | 45.034 | 54.378 | 1.00 | 42.73 | C |
| ATOM | 2025 | CG1 | VAL | F | 182 | −2.483 | 44.019 | 53.821 | 1.00 | 43.26 | C |
| ATOM | 2029 | CG2 | VAL | F | 182 | −0.069 | 44.471 | 54.330 | 1.00 | 43.17 | C |
| ATOM | 2033 | C | VAL | F | 182 | −3.017 | 46.845 | 53.515 | 1.00 | 43.74 | C |
| ATOM | 2034 | O | VAL | F | 182 | −3.704 | 46.965 | 54.531 | 1.00 | 41.25 | O |
| ATOM | 2036 | N | LEU | F | 183 | −3.456 | 47.144 | 52.300 | 1.00 | 47.76 | N |
| ATOM | 2037 | CA | LEU | F | 183 | −4.773 | 47.717 | 52.073 | 1.00 | 51.36 | C |
| ATOM | 2039 | CB | LEU | F | 183 | −4.670 | 48.895 | 51.089 | 1.00 | 53.23 | C |
| ATOM | 2042 | CG | LEU | F | 183 | −3.793 | 50.065 | 51.577 | 1.00 | 53.38 | C |
| ATOM | 2044 | CD1 | LEU | F | 183 | −3.572 | 51.095 | 50.473 | 1.00 | 53.94 | C |
| ATOM | 2048 | CD2 | LEU | F | 183 | −4.386 | 50.730 | 52.816 | 1.00 | 51.39 | C |
| ATOM | 2052 | C | LEU | F | 183 | −5.745 | 46.650 | 51.569 | 1.00 | 53.53 | C |
| ATOM | 2053 | O | LEU | F | 183 | −5.385 | 45.743 | 50.815 | 1.00 | 51.68 | O |
| ATOM | 2055 | OXT | LEU | F | 183 | −6.922 | 46.671 | 51.934 | 1.00 | 57.05 | O |
| ATOM | 2056 | C1 | NAG | F | 200 | −4.868 | 42.259 | 80.901 | 1.00 | 61.02 | C |
| ATOM | 2059 | C2 | NAG | F | 200 | −3.908 | 41.258 | 80.253 | 1.00 | 62.06 | C |
| ATOM | 2061 | N2 | NAG | F | 200 | −4.564 | 40.490 | 79.199 | 1.00 | 61.70 | N |
| ATOM | 2063 | C7 | NAG | F | 200 | −4.235 | 40.539 | 77.902 | 1.00 | 63.68 | C |
| ATOM | 2064 | O7 | NAG | F | 200 | −5.075 | 40.461 | 77.010 | 1.00 | 63.74 | O |
| ATOM | 2065 | C8 | NAG | F | 200 | −2.786 | 40.658 | 77.524 | 1.00 | 65.63 | C |
| ATOM | 2069 | C3 | NAG | F | 200 | −3.357 | 40.322 | 81.330 | 1.00 | 64.43 | C |
| ATOM | 2071 | O3 | NAG | F | 200 | −2.371 | 39.458 | 80.797 | 1.00 | 62.44 | O |
| ATOM | 2073 | C4 | NAG | F | 200 | −2.765 | 41.109 | 82.503 | 1.00 | 66.42 | C |
| ATOM | 2075 | O4 | NAG | F | 200 | −2.493 | 40.232 | 83.582 | 1.00 | 67.74 | O |
| ATOM | 2077 | C5 | NAG | F | 200 | −3.676 | 42.255 | 82.966 | 1.00 | 65.41 | C |
| ATOM | 2079 | C6 | NAG | F | 200 | −2.932 | 43.179 | 83.936 | 1.00 | 65.22 | C |
| ATOM | 2082 | O6 | NAG | F | 200 | −3.776 | 43.621 | 84.979 | 1.00 | 64.02 | O |
| ATOM | 2084 | O5 | NAG | F | 200 | −4.131 | 43.011 | 81.852 | 1.00 | 62.60 | O |
| ATOM | 2085 | N | LEU | R | 29 | −4.590 | 37.427 | 47.176 | 1.00 | 68.49 | N |
| ATOM | 2086 | CA | LEU | R | 29 | −3.361 | 37.040 | 47.937 | 1.00 | 67.62 | C |
| ATOM | 2088 | CB | LEU | R | 29 | −2.271 | 38.107 | 47.782 | 1.00 | 67.85 | C |
| ATOM | 2091 | CG | LEU | R | 29 | −0.922 | 37.802 | 48.442 | 1.00 | 67.30 | C |
| ATOM | 2093 | CD1 | LEU | R | 29 | −0.334 | 36.496 | 47.905 | 1.00 | 66.86 | C |
| ATOM | 2097 | CD2 | LEU | R | 29 | 0.035 | 38.961 | 48.226 | 1.00 | 67.01 | C |
| ATOM | 2101 | C | LEU | R | 29 | −3.658 | 36.840 | 49.420 | 1.00 | 65.77 | C |
| ATOM | 2102 | O | LEU | R | 29 | −4.113 | 37.763 | 50.094 | 1.00 | 65.27 | O |
| ATOM | 2106 | N | HIS | R | 30 | −3.383 | 35.637 | 49.920 | 1.00 | 64.96 | N |
| ATOM | 2107 | CA | HIS | R | 30 | −3.589 | 35.318 | 51.331 | 1.00 | 64.21 | C |
| ATOM | 2109 | CB | HIS | R | 30 | −4.815 | 34.416 | 51.504 | 1.00 | 68.47 | C |
| ATOM | 2112 | CG | HIS | R | 30 | −5.786 | 34.914 | 52.529 | 1.00 | 71.12 | C |
| ATOM | 2113 | ND1 | HIS | R | 30 | −6.976 | 35.521 | 52.190 | 1.00 | 72.21 | N |
| ATOM | 2115 | CE1 | HIS | R | 30 | −7.622 | 35.863 | 53.291 | 1.00 | 72.83 | C |
| ATOM | 2117 | NE2 | HIS | R | 30 | −6.891 | 35.505 | 54.332 | 1.00 | 73.27 | N |
| ATOM | 2119 | CD2 | HIS | R | 30 | −5.736 | 34.910 | 53.883 | 1.00 | 72.51 | C |
| ATOM | 2121 | C | HIS | R | 30 | −2.336 | 34.650 | 51.896 | 1.00 | 62.07 | C |
| ATOM | 2122 | O | HIS | R | 30 | −2.014 | 33.510 | 51.546 | 1.00 | 60.28 | O |
| ATOM | 2124 | N | CYS | R | 31 | −1.632 | 35.379 | 52.761 | 1.00 | 59.01 | N |
| ATOM | 2125 | CA | CYS | R | 31 | −0.369 | 34.927 | 53.333 | 1.00 | 56.06 | C |
| ATOM | 2127 | CB | CYS | R | 31 | 0.576 | 36.111 | 53.532 | 1.00 | 49.48 | C |
| ATOM | 2130 | SG | CYS | R | 31 | 1.007 | 36.978 | 52.023 | 1.00 | 41.00 | S |
| ATOM | 2132 | C | CYS | R | 31 | −0.614 | 34.257 | 54.672 | 1.00 | 57.87 | C |
| ATOM | 2133 | O | CYS | R | 31 | −1.389 | 34.762 | 55.489 | 1.00 | 60.13 | O |
| ATOM | 2135 | N | VAL | R | 32 | 0.058 | 33.131 | 54.901 | 1.00 | 56.89 | N |
| ATOM | 2136 | CA | VAL | R | 32 | −0.114 | 32.369 | 56.136 | 1.00 | 54.65 | C |
| ATOM | 2138 | CB | VAL | R | 32 | −0.649 | 30.941 | 55.867 | 1.00 | 56.56 | C |
| ATOM | 2140 | CG1 | VAL | R | 32 | −2.115 | 31.001 | 55.461 | 1.00 | 57.47 | C |
| ATOM | 2144 | CG2 | VAL | R | 32 | 0.185 | 30.221 | 54.803 | 1.00 | 57.11 | C |
| ATOM | 2148 | C | VAL | R | 32 | 1.185 | 32.284 | 56.925 | 1.00 | 53.50 | C |
| ATOM | 2149 | O | VAL | R | 32 | 2.280 | 32.297 | 56.355 | 1.00 | 51.88 | O |
| ATOM | 2151 | N | GLY | R | 33 | 1.047 | 32.197 | 58.244 | 1.00 | 51.87 | N |
| ATOM | 2152 | CA | GLY | R | 33 | 2.192 | 32.066 | 59.129 | 1.00 | 49.61 | C |
| ATOM | 2155 | C | GLY | R | 33 | 2.946 | 33.371 | 59.273 | 1.00 | 48.76 | C |
| ATOM | 2156 | O | GLY | R | 33 | 2.339 | 34.428 | 59.475 | 1.00 | 50.34 | O |
| ATOM | 2158 | N | ASP | R | 34 | 4.270 | 33.295 | 59.145 | 1.00 | 45.33 | N |
| ATOM | 2159 | CA | ASP | R | 34 | 5.159 | 34.417 | 59.450 | 1.00 | 41.78 | C |
| ATOM | 2161 | CB | ASP | R | 34 | 6.370 | 33.902 | 60.212 | 1.00 | 39.78 | C |
| ATOM | 2164 | CG | ASP | R | 34 | 5.988 | 33.233 | 61.517 | 1.00 | 41.19 | C |
| ATOM | 2165 | OD1 | ASP | R | 34 | 5.015 | 33.661 | 62.170 | 1.00 | 45.29 | O |
| ATOM | 2166 | OD2 | ASP | R | 34 | 6.672 | 32.279 | 61.907 | 1.00 | 41.33 | O |
| ATOM | 2167 | C | ASP | R | 34 | 5.586 | 35.181 | 58.195 | 1.00 | 40.52 | C |
| ATOM | 2168 | O | ASP | R | 34 | 6.772 | 35.414 | 57.954 | 1.00 | 38.72 | O |
| ATOM | 2170 | N | THR | R | 35 | 4.585 | 35.574 | 57.413 | 1.00 | 40.50 | N |
| ATOM | 2171 | CA | THR | R | 35 | 4.774 | 36.348 | 56.205 | 1.00 | 37.48 | C |
| ATOM | 2173 | CB | THR | R | 35 | 4.564 | 35.477 | 54.934 | 1.00 | 37.55 | C |
| ATOM | 2175 | OG1 | THR | R | 35 | 3.261 | 34.884 | 54.956 | 1.00 | 36.83 | O |
| ATOM | 2177 | CG2 | THR | R | 35 | 5.606 | 34.368 | 54.845 | 1.00 | 37.16 | C |
| ATOM | 2181 | C | THR | R | 35 | 3.781 | 37.513 | 56.202 | 1.00 | 37.67 | C |
| ATOM | 2182 | O | THR | R | 35 | 2.865 | 37.564 | 57.029 | 1.00 | 34.55 | O |
| ATOM | 2184 | N | TYR | R | 36 | 3.983 | 38.453 | 55.283 | 1.00 | 37.27 | N |
| ATOM | 2185 | CA | TYR | R | 36 | 3.035 | 39.543 | 55.051 | 1.00 | 36.18 | C |
| ATOM | 2187 | CB | TYR | R | 36 | 3.475 | 40.827 | 55.757 | 1.00 | 34.24 | C |
| ATOM | 2190 | CG | TYR | R | 36 | 4.856 | 41.318 | 55.379 | 1.00 | 33.51 | C |
| ATOM | 2191 | CD1 | TYR | R | 36 | 5.037 | 42.233 | 54.347 | 1.00 | 33.30 | C |
| ATOM | 2193 | CE1 | TYR | R | 36 | 6.306 | 42.684 | 54.001 | 1.00 | 33.44 | C |
| ATOM | 2195 | CZ | TYR | R | 36 | 7.409 | 42.230 | 54.702 | 1.00 | 33.51 | C |
| ATOM | 2196 | OH | TYR | R | 36 | 8.668 | 42.676 | 54.380 | 1.00 | 32.68 | O |
| ATOM | 2198 | CE2 | TYR | R | 36 | 7.255 | 41.328 | 55.736 | 1.00 | 34.65 | C |
| ATOM | 2200 | CD2 | TYR | R | 36 | 5.984 | 40.874 | 56.068 | 1.00 | 34.20 | C |
| ATOM | 2202 | C | TYR | R | 36 | 2.921 | 39.805 | 53.563 | 1.00 | 35.86 | C |
| ATOM | 2203 | O | TYR | R | 36 | 3.893 | 39.604 | 52.824 | 1.00 | 35.42 | O |
| ATOM | 2205 | N | PRO | R | 37 | 1.738 | 40.265 | 53.116 | 1.00 | 35.59 | N |
| ATOM | 2206 | CA | PRO | R | 37 | 1.528 | 40.542 | 51.702 | 1.00 | 35.78 | C |
| ATOM | 2208 | CB | PRO | R | 37 | −0.001 | 40.624 | 51.582 | 1.00 | 34.57 | C |
| ATOM | 2211 | CG | PRO | R | 37 | −0.476 | 41.014 | 52.909 | 1.00 | 33.07 | C |
| ATOM | 2214 | CD | PRO | R | 37 | 0.531 | 40.536 | 53.917 | 1.00 | 34.42 | C |
| ATOM | 2217 | C | PRO | R | 37 | 2.187 | 41.839 | 51.240 | 1.00 | 34.31 | C |
| ATOM | 2218 | O | PRO | R | 37 | 1.989 | 42.881 | 51.852 | 1.00 | 35.70 | O |
| ATOM | 2219 | N | SER | R | 38 | 2.968 | 41.751 | 50.168 | 1.00 | 37.15 | N |
| ATOM | 2220 | CA | SER | R | 38 | 3.566 | 42.911 | 49.519 | 1.00 | 39.54 | C |
| ATOM | 2222 | CB | SER | R | 38 | 4.827 | 43.353 | 50.258 | 1.00 | 40.87 | C |
| ATOM | 2225 | OG | SER | R | 38 | 5.424 | 44.482 | 49.637 | 1.00 | 39.37 | O |
| ATOM | 2227 | C | SER | R | 38 | 3.921 | 42.549 | 48.085 | 1.00 | 42.65 | C |
| ATOM | 2228 | O | SER | R | 38 | 4.465 | 41.476 | 47.832 | 1.00 | 44.60 | O |
| ATOM | 2230 | N | ASN | R | 39 | 3.600 | 43.438 | 47.148 | 1.00 | 46.63 | N |
| ATOM | 2231 | CA | ASN | R | 39 | 3.961 | 43.251 | 45.745 | 1.00 | 47.22 | C |
| ATOM | 2233 | CB | ASN | R | 39 | 5.493 | 43.324 | 45.583 | 1.00 | 49.37 | C |
| ATOM | 2236 | CG | ASN | R | 39 | 6.074 | 44.656 | 46.046 | 1.00 | 50.52 | C |
| ATOM | 2237 | OD1 | ASN | R | 39 | 6.702 | 44.747 | 47.109 | 1.00 | 50.59 | O |
| ATOM | 2238 | ND2 | ASN | R | 39 | 5.867 | 45.698 | 45.246 | 1.00 | 50.40 | N |
| ATOM | 2241 | C | ASN | R | 39 | 3.422 | 41.932 | 45.170 | 1.00 | 48.67 | C |
| ATOM | 2242 | O | ASN | R | 39 | 4.160 | 41.181 | 44.525 | 1.00 | 51.24 | O |
| ATOM | 2244 | N | ASP | R | 40 | 2.139 | 41.657 | 45.414 | 1.00 | 47.61 | N |
| ATOM | 2245 | CA | ASP | R | 40 | 1.489 | 40.413 | 44.958 | 1.00 | 49.01 | C |
| ATOM | 2247 | CB | ASP | R | 40 | 1.292 | 40.426 | 43.432 | 1.00 | 50.99 | C |
| ATOM | 2250 | CG | ASP | R | 40 | 0.543 | 41.660 | 42.938 | 1.00 | 54.43 | C |
| ATOM | 2251 | OD1 | ASP | R | 40 | 0.115 | 42.490 | 43.771 | 1.00 | 55.76 | O |
| ATOM | 2252 | OD2 | ASP | R | 40 | 0.380 | 41.798 | 41.702 | 1.00 | 56.84 | O |
| ATOM | 2253 | C | ASP | R | 40 | 2.264 | 39.150 | 45.378 | 1.00 | 48.84 | C |
| ATOM | 2254 | O | ASP | R | 40 | 2.331 | 38.172 | 44.638 | 1.00 | 49.49 | O |
| ATOM | 2256 | N | ARG | R | 41 | 2.839 | 39.180 | 46.576 | 1.00 | 49.68 | N |
| ATOM | 2257 | CA | ARG | R | 41 | 3.682 | 38.097 | 47.071 | 1.00 | 47.71 | C |
| ATOM | 2259 | CB | ARG | R | 41 | 5.094 | 38.224 | 46.477 | 1.00 | 49.94 | C |
| ATOM | 2262 | CG | ARG | R | 41 | 6.223 | 37.532 | 47.262 | 1.00 | 50.35 | C |
| ATOM | 2265 | CD | ARG | R | 41 | 7.584 | 37.754 | 46.617 | 1.00 | 50.24 | C |
| ATOM | 2268 | NE | ARG | R | 41 | 7.944 | 39.176 | 46.579 | 1.00 | 51.57 | N |
| ATOM | 2270 | CZ | ARG | R | 41 | 9.027 | 39.724 | 47.131 | 1.00 | 49.92 | C |
| ATOM | 2271 | NH1 | ARG | R | 41 | 9.925 | 38.999 | 47.779 | 1.00 | 50.09 | N |
| ATOM | 2274 | NH2 | ARG | R | 41 | 9.224 | 41.022 | 47.013 | 1.00 | 50.40 | N |
| ATOM | 2277 | C | ARG | R | 41 | 3.718 | 38.158 | 48.593 | 1.00 | 45.51 | C |
| ATOM | 2278 | O | ARG | R | 41 | 3.420 | 39.195 | 49.176 | 1.00 | 43.63 | O |
| ATOM | 2280 | N | CYS | R | 42 | 4.074 | 37.037 | 49.219 | 1.00 | 42.92 | N |
| ATOM | 2281 | CA | CYS | R | 42 | 4.185 | 36.937 | 50.663 | 1.00 | 40.88 | C |
| ATOM | 2283 | CB | CYS | R | 42 | 3.560 | 35.624 | 51.132 | 1.00 | 40.54 | C |
| ATOM | 2286 | SG | CYS | R | 42 | 1.775 | 35.525 | 50.830 | 1.00 | 41.29 | S |
| ATOM | 2288 | C | CYS | R | 42 | 5.642 | 37.026 | 51.127 | 1.00 | 41.63 | C |
| ATOM | 2289 | O | CYS | R | 42 | 6.403 | 36.069 | 50.966 | 1.00 | 42.56 | O |
| ATOM | 2291 | N | CYS | R | 43 | 6.016 | 38.172 | 51.709 | 1.00 | 39.71 | N |
| ATOM | 2292 | CA | CYS | R | 43 | 7.374 | 38.396 | 52.222 | 1.00 | 37.60 | C |
| ATOM | 2294 | CB | CYS | R | 43 | 7.764 | 39.863 | 52.084 | 1.00 | 39.25 | C |
| ATOM | 2297 | SG | CYS | R | 43 | 7.924 | 40.486 | 50.392 | 1.00 | 40.36 | S |
| ATOM | 2299 | C | CYS | R | 43 | 7.488 | 37.980 | 53.691 | 1.00 | 37.12 | C |
| ATOM | 2300 | O | CYS | R | 43 | 6.491 | 37.916 | 54.401 | 1.00 | 37.00 | O |
| ATOM | 2302 | N | HIS | R | 44 | 8.710 | 37.730 | 54.153 | 1.00 | 34.87 | N |
| ATOM | 2303 | CA | HIS | R | 44 | 8.914 | 37.096 | 55.451 | 1.00 | 35.08 | C |
| ATOM | 2305 | CB | HIS | R | 44 | 9.999 | 36.015 | 55.365 | 1.00 | 32.44 | C |
| ATOM | 2308 | CG | HIS | R | 44 | 9.522 | 34.738 | 54.748 | 1.00 | 32.05 | C |
| ATOM | 2309 | ND1 | HIS | R | 44 | 9.460 | 34.546 | 53.387 | 1.00 | 31.26 | N |
| ATOM | 2311 | CE1 | HIS | R | 44 | 8.996 | 33.335 | 53.134 | 1.00 | 33.71 | C |
| ATOM | 2313 | NE2 | HIS | R | 44 | 8.746 | 32.736 | 54.284 | 1.00 | 33.11 | N |
| ATOM | 2315 | CD2 | HIS | R | 44 | 9.062 | 33.594 | 55.310 | 1.00 | 34.02 | C |
| ATOM | 2317 | C | HIS | R | 44 | 9.250 | 38.087 | 56.558 | 1.00 | 39.44 | C |
| ATOM | 2318 | O | HIS | R | 44 | 10.018 | 39.037 | 56.371 | 1.00 | 38.28 | O |
| ATOM | 2320 | N | GLU | R | 45 | 8.655 | 37.858 | 57.722 | 1.00 | 39.77 | N |
| ATOM | 2321 | CA | GLU | R | 45 | 8.992 | 38.633 | 58.893 | 1.00 | 40.74 | C |
| ATOM | 2323 | CB | GLU | R | 45 | 7.917 | 38.476 | 59.962 | 1.00 | 43.89 | C |
| ATOM | 2326 | CG | GLU | R | 45 | 6.616 | 39.132 | 59.542 | 1.00 | 47.80 | C |
| ATOM | 2329 | CD | GLU | R | 45 | 5.579 | 39.056 | 60.612 | 1.00 | 52.76 | C |
| ATOM | 2330 | OE1 | GLU | R | 45 | 5.041 | 37.939 | 60.817 | 1.00 | 53.75 | O |
| ATOM | 2331 | OE2 | GLU | R | 45 | 5.319 | 40.110 | 61.260 | 1.00 | 62.14 | O |
| ATOM | 2332 | C | GLU | R | 45 | 10.362 | 38.229 | 59.409 | 1.00 | 37.15 | C |
| ATOM | 2333 | O | GLU | R | 45 | 10.914 | 37.181 | 59.040 | 1.00 | 32.72 | O |
| ATOM | 2335 | N | CYS | R | 46 | 10.930 | 39.097 | 60.231 | 1.00 | 37.98 | N |
| ATOM | 2336 | CA | CYS | R | 46 | 12.261 | 38.865 | 60.750 | 1.00 | 39.28 | C |
| ATOM | 2338 | CB | CYS | R | 46 | 12.904 | 40.171 | 61.238 | 1.00 | 41.93 | C |
| ATOM | 2341 | SG | CYS | R | 46 | 13.212 | 41.364 | 59.891 | 1.00 | 40.71 | S |
| ATOM | 2343 | C | CYS | R | 46 | 12.170 | 37.838 | 61.850 | 1.00 | 37.19 | C |
| ATOM | 2344 | O | CYS | R | 46 | 11.108 | 37.639 | 62.445 | 1.00 | 36.08 | O |
| ATOM | 2346 | N | ARG | R | 47 | 13.299 | 37.185 | 62.091 | 1.00 | 37.01 | N |
| ATOM | 2347 | CA | ARG | R | 47 | 13.401 | 36.094 | 63.044 | 1.00 | 37.38 | C |
| ATOM | 2349 | CB | ARG | R | 47 | 14.290 | 35.012 | 62.437 | 1.00 | 36.98 | C |
| ATOM | 2352 | CG | ARG | R | 47 | 13.595 | 34.301 | 61.297 | 1.00 | 35.26 | C |
| ATOM | 2355 | CD | ARG | R | 47 | 12.657 | 33.302 | 61.899 | 1.00 | 37.58 | C |
| ATOM | 2358 | NE | ARG | R | 47 | 11.292 | 33.386 | 61.439 | 1.00 | 34.84 | N |
| ATOM | 2360 | CZ | ARG | R | 47 | 10.336 | 32.565 | 61.852 | 1.00 | 34.86 | C |
| ATOM | 2361 | NH1 | ARG | R | 47 | 10.581 | 31.608 | 62.734 | 1.00 | 35.21 | N |
| ATOM | 2364 | NH2 | ARG | R | 47 | 9.117 | 32.689 | 61.371 | 1.00 | 38.90 | N |
| ATOM | 2367 | C | ARG | R | 47 | 13.994 | 36.583 | 64.354 | 1.00 | 37.63 | C |
| ATOM | 2368 | O | ARG | R | 47 | 14.623 | 37.635 | 64.383 | 1.00 | 39.92 | O |
| ATOM | 2370 | N | PRO | R | 48 | 13.798 | 35.818 | 65.449 | 1.00 | 38.02 | N |
| ATOM | 2371 | CA | PRO | R | 48 | 14.403 | 36.164 | 66.730 | 1.00 | 37.12 | C |
| ATOM | 2373 | CB | PRO | R | 48 | 14.069 | 34.952 | 67.604 | 1.00 | 38.82 | C |
| ATOM | 2376 | CG | PRO | R | 48 | 12.812 | 34.431 | 67.026 | 1.00 | 37.34 | C |
| ATOM | 2379 | CD | PRO | R | 48 | 12.985 | 34.592 | 65.557 | 1.00 | 37.25 | C |
| ATOM | 2382 | C | PRO | R | 48 | 15.908 | 36.353 | 66.606 | 1.00 | 36.34 | C |
| ATOM | 2383 | O | PRO | R | 48 | 16.554 | 35.631 | 65.849 | 1.00 | 39.58 | O |
| ATOM | 2384 | N | GLY | R | 49 | 16.451 | 37.329 | 67.330 | 1.00 | 36.52 | N |
| ATOM | 2385 | CA | GLY | R | 49 | 17.856 | 37.708 | 67.204 | 1.00 | 36.12 | C |
| ATOM | 2388 | C | GLY | R | 49 | 18.134 | 38.623 | 66.020 | 1.00 | 38.32 | C |
| ATOM | 2389 | O | GLY | R | 49 | 19.301 | 38.891 | 65.692 | 1.00 | 35.18 | O |
| ATOM | 2391 | N | ASN | R | 50 | 17.067 | 39.087 | 65.369 | 1.00 | 37.15 | N |
| ATOM | 2392 | CA | ASN | R | 50 | 17.171 | 40.012 | 64.246 | 1.00 | 37.85 | C |
| ATOM | 2394 | CB | ASN | R | 50 | 17.044 | 39.295 | 62.892 | 1.00 | 35.71 | C |
| ATOM | 2397 | CG | ASN | R | 50 | 18.068 | 38.192 | 62.700 | 1.00 | 34.18 | C |
| ATOM | 2398 | OD1 | ASN | R | 50 | 19.134 | 38.404 | 62.109 | 1.00 | 32.50 | O |
| ATOM | 2399 | ND2 | ASN | R | 50 | 17.741 | 36.998 | 63.180 | 1.00 | 32.35 | N |
| ATOM | 2402 | C | ASN | R | 50 | 16.098 | 41.097 | 64.334 | 1.00 | 38.00 | C |
| ATOM | 2403 | O | ASN | R | 50 | 15.043 | 40.900 | 64.939 | 1.00 | 39.01 | O |
| ATOM | 2405 | N | GLY | R | 51 | 16.383 | 42.236 | 63.713 | 1.00 | 37.06 | N |
| ATOM | 2406 | CA | GLY | R | 51 | 15.444 | 43.341 | 63.642 | 1.00 | 37.50 | C |
| ATOM | 2409 | C | GLY | R | 51 | 15.237 | 43.761 | 62.207 | 1.00 | 36.01 | C |
| ATOM | 2410 | O | GLY | R | 51 | 16.136 | 43.624 | 61.375 | 1.00 | 33.03 | O |
| ATOM | 2412 | N | MET | R | 52 | 14.043 | 44.275 | 61.920 | 1.00 | 36.80 | N |
| ATOM | 2413 | CA | MET | R | 52 | 13.700 | 44.719 | 60.584 | 1.00 | 35.50 | C |
| ATOM | 2415 | CB | MET | R | 52 | 12.192 | 44.681 | 60.354 | 1.00 | 36.97 | C |
| ATOM | 2418 | CG | MET | R | 52 | 11.828 | 44.725 | 58.868 | 1.00 | 36.34 | C |
| ATOM | 2421 | SD | MET | R | 52 | 10.079 | 44.914 | 58.530 | 1.00 | 35.02 | S |
| ATOM | 2422 | CE | MET | R | 52 | 9.826 | 46.642 | 58.945 | 1.00 | 35.14 | C |
| ATOM | 2426 | C | MET | R | 52 | 14.219 | 46.119 | 60.332 | 1.00 | 35.44 | C |
| ATOM | 2427 | O | MET | R | 52 | 13.903 | 47.047 | 61.072 | 1.00 | 36.41 | O |
| ATOM | 2429 | N | VAL | R | 53 | 15.021 | 46.241 | 59.274 | 1.00 | 36.38 | N |
| ATOM | 2430 | CA | VAL | R | 53 | 15.573 | 47.499 | 58.816 | 1.00 | 35.10 | C |
| ATOM | 2432 | CB | VAL | R | 53 | 16.958 | 47.269 | 58.135 | 1.00 | 37.13 | C |
| ATOM | 2434 | CG1 | VAL | R | 53 | 17.490 | 48.557 | 57.495 | 1.00 | 36.05 | C |
| ATOM | 2438 | CG2 | VAL | R | 53 | 17.966 | 46.694 | 59.139 | 1.00 | 37.55 | C |
| ATOM | 2442 | C | VAL | R | 53 | 14.595 | 48.128 | 57.818 | 1.00 | 36.17 | C |
| ATOM | 2443 | O | VAL | R | 53 | 14.332 | 49.330 | 57.867 | 1.00 | 30.66 | O |
| ATOM | 2445 | N | SER | R | 54 | 14.084 | 47.303 | 56.902 | 1.00 | 36.58 | N |
| ATOM | 2446 | CA | SER | R | 54 | 13.094 | 47.740 | 55.933 | 1.00 | 35.77 | C |
| ATOM | 2448 | CB | SER | R | 54 | 13.765 | 48.460 | 54.751 | 1.00 | 37.90 | C |
| ATOM | 2451 | OG | SER | R | 54 | 14.694 | 47.635 | 54.073 | 1.00 | 38.42 | O |
| ATOM | 2453 | C | SER | R | 54 | 12.262 | 46.564 | 55.443 | 1.00 | 36.21 | C |
| ATOM | 2454 | O | SER | R | 54 | 12.689 | 45.417 | 55.510 | 1.00 | 39.97 | O |
| ATOM | 2456 | N | ARG | R | 55 | 11.061 | 46.868 | 54.962 | 1.00 | 38.21 | N |
| ATOM | 2457 | CA | ARG | R | 55 | 10.186 | 45.883 | 54.329 | 1.00 | 37.59 | C |
| ATOM | 2459 | CB | ARG | R | 55 | 8.799 | 46.499 | 54.069 | 1.00 | 38.54 | C |
| ATOM | 2462 | CG | ARG | R | 55 | 7.976 | 46.824 | 55.321 | 1.00 | 37.24 | C |
| ATOM | 2465 | CD | ARG | R | 55 | 7.220 | 45.602 | 55.823 | 1.00 | 38.17 | C |
| ATOM | 2468 | NE | ARG | R | 55 | 6.392 | 45.896 | 56.992 | 1.00 | 38.99 | N |
| ATOM | 2470 | CZ | ARG | R | 55 | 5.861 | 44.982 | 57.805 | 1.00 | 38.46 | C |
| ATOM | 2471 | NH1 | ARG | R | 55 | 6.051 | 43.691 | 57.600 | 1.00 | 40.31 | N |
| ATOM | 2474 | NH2 | ARG | R | 55 | 5.136 | 45.363 | 58.844 | 1.00 | 39.89 | N |
| ATOM | 2477 | C | ARG | R | 55 | 10.781 | 45.421 | 53.003 | 1.00 | 38.59 | C |
| ATOM | 2478 | O | ARG | R | 55 | 11.657 | 46.070 | 52.435 | 1.00 | 40.77 | O |
| ATOM | 2480 | N | CYS | R | 56 | 10.271 | 44.308 | 52.495 | 1.00 | 38.66 | N |
| ATOM | 2481 | CA | CYS | R | 56 | 10.747 | 43.752 | 51.243 | 1.00 | 40.38 | C |
| ATOM | 2483 | CB | CYS | R | 56 | 10.118 | 42.386 | 51.012 | 1.00 | 39.15 | C |
| ATOM | 2486 | SG | CYS | R | 56 | 8.367 | 42.445 | 50.673 | 1.00 | 37.51 | S |
| ATOM | 2488 | C | CYS | R | 56 | 10.444 | 44.655 | 50.043 | 1.00 | 43.13 | C |
| ATOM | 2489 | O | CYS | R | 56 | 9.464 | 45.403 | 50.040 | 1.00 | 42.16 | O |
| ATOM | 2491 | N | SER | R | 57 | 11.307 | 44.565 | 49.033 | 1.00 | 45.93 | N |
| ATOM | 2492 | CA | SER | R | 57 | 11.159 | 45.303 | 47.775 | 1.00 | 46.60 | C |
| ATOM | 2494 | CB | SER | R | 57 | 12.531 | 45.759 | 47.280 | 1.00 | 44.57 | C |
| ATOM | 2497 | OG | SER | R | 57 | 13.351 | 44.642 | 46.982 | 1.00 | 38.85 | O |
| ATOM | 2499 | C | SER | R | 57 | 10.497 | 44.391 | 46.741 | 1.00 | 48.81 | C |
| ATOM | 2500 | O | SER | R | 57 | 9.982 | 43.330 | 47.084 | 1.00 | 47.48 | O |
| ATOM | 2502 | N | ARG | R | 58 | 10.497 | 44.803 | 45.480 | 1.00 | 50.54 | N |
| ATOM | 2503 | CA | ARG | R | 58 | 9.923 | 43.981 | 44.419 | 1.00 | 51.31 | C |
| ATOM | 2505 | CB | ARG | R | 58 | 9.904 | 44.750 | 43.110 | 1.00 | 54.51 | C |
| ATOM | 2508 | CG | ARG | R | 58 | 9.005 | 45.972 | 43.048 | 1.00 | 57.59 | C |
| ATOM | 2511 | CD | ARG | R | 58 | 9.139 | 46.667 | 41.686 | 1.00 | 58.60 | C |
| ATOM | 2514 | NE | ARG | R | 58 | 8.803 | 45.769 | 40.578 | 1.00 | 61.06 | N |
| ATOM | 2516 | CZ | ARG | R | 58 | 9.648 | 44.929 | 39.970 | 1.00 | 63.31 | C |
| ATOM | 2517 | NH1 | ARG | R | 58 | 10.929 | 44.834 | 40.336 | 1.00 | 63.39 | N |
| ATOM | 2520 | NH2 | ARG | R | 58 | 9.206 | 44.165 | 38.978 | 1.00 | 63.48 | N |
| ATOM | 2523 | C | ARG | R | 58 | 10.707 | 42.691 | 44.181 | 1.00 | 49.28 | C |
| ATOM | 2524 | O | ARG | R | 58 | 10.136 | 41.686 | 43.757 | 1.00 | 51.57 | O |
| ATOM | 2526 | N | SER | R | 59 | 12.015 | 42.736 | 44.422 | 1.00 | 46.83 | N |
| ATOM | 2527 | CA | SER | R | 59 | 12.913 | 41.628 | 44.097 | 1.00 | 49.60 | C |
| ATOM | 2529 | CB | SER | R | 59 | 14.040 | 42.145 | 43.211 | 1.00 | 53.62 | C |
| ATOM | 2532 | OG | SER | R | 59 | 14.675 | 43.263 | 43.816 | 1.00 | 57.66 | O |
| ATOM | 2534 | C | SER | R | 59 | 13.530 | 40.945 | 45.314 | 1.00 | 47.99 | C |
| ATOM | 2535 | O | SER | R | 59 | 13.820 | 39.749 | 45.271 | 1.00 | 48.35 | O |
| ATOM | 2537 | N | GLN | R | 60 | 13.754 | 41.712 | 46.376 | 1.00 | 45.52 | N |
| ATOM | 2538 | CA | GLN | R | 60 | 14.411 | 41.216 | 47.581 | 1.00 | 47.39 | C |
| ATOM | 2540 | CB | GLN | R | 60 | 15.410 | 42.254 | 48.115 | 1.00 | 50.40 | C |
| ATOM | 2543 | CG | GLN | R | 60 | 16.318 | 42.891 | 47.055 | 1.00 | 53.71 | C |
| ATOM | 2546 | CD | GLN | R | 60 | 17.219 | 41.884 | 46.356 | 1.00 | 55.99 | C |
| ATOM | 2547 | OE1 | GLN | R | 60 | 17.641 | 40.889 | 46.952 | 1.00 | 55.32 | O |
| ATOM | 2548 | NE2 | GLN | R | 60 | 17.529 | 42.145 | 45.086 | 1.00 | 58.04 | N |
| ATOM | 2551 | C | GLN | R | 60 | 13.376 | 40.914 | 48.659 | 1.00 | 46.37 | C |
| ATOM | 2552 | O | GLN | R | 60 | 12.260 | 41.435 | 48.634 | 1.00 | 44.21 | O |
| ATOM | 2554 | N | ASN | R | 61 | 13.760 | 40.069 | 49.608 | 1.00 | 47.09 | N |
| ATOM | 2555 | CA | ASN | R | 61 | 12.932 | 39.792 | 50.771 | 1.00 | 45.68 | C |
| ATOM | 2557 | CB | ASN | R | 61 | 13.236 | 38.391 | 51.331 | 1.00 | 47.62 | C |
| ATOM | 2560 | CG | ASN | R | 61 | 12.043 | 37.767 | 52.076 | 1.00 | 48.04 | C |
| ATOM | 2561 | OD1 | ASN | R | 61 | 11.186 | 38.471 | 52.610 | 1.00 | 44.26 | O |
| ATOM | 2562 | ND2 | ASN | R | 61 | 11.991 | 36.434 | 52.100 | 1.00 | 46.39 | N |
| ATOM | 2565 | C | ASN | R | 61 | 13.205 | 40.876 | 51.812 | 1.00 | 43.93 | C |
| ATOM | 2566 | O | ASN | R | 61 | 14.088 | 41.717 | 51.636 | 1.00 | 43.05 | O |
| ATOM | 2568 | N | THR | R | 62 | 12.429 | 40.851 | 52.886 | 1.00 | 41.64 | N |
| ATOM | 2569 | CA | THR | R | 62 | 12.619 | 41.730 | 54.033 | 1.00 | 40.65 | C |
| ATOM | 2571 | CB | THR | R | 62 | 11.822 | 41.178 | 55.233 | 1.00 | 38.78 | C |
| ATOM | 2573 | OG1 | THR | R | 62 | 10.498 | 40.832 | 54.799 | 1.00 | 37.94 | O |
| ATOM | 2575 | CG2 | THR | R | 62 | 11.741 | 42.188 | 56.358 | 1.00 | 39.65 | C |
| ATOM | 2579 | C | THR | R | 62 | 14.092 | 41.864 | 54.436 | 1.00 | 40.08 | C |
| ATOM | 2580 | O | THR | R | 62 | 14.813 | 40.869 | 54.502 | 1.00 | 42.46 | O |
| ATOM | 2582 | N | VAL | R | 63 | 14.533 | 43.092 | 54.706 | 1.00 | 39.61 | N |
| ATOM | 2583 | CA | VAL | R | 63 | 15.892 | 43.318 | 55.199 | 1.00 | 39.02 | C |
| ATOM | 2585 | CB | VAL | R | 63 | 16.489 | 44.656 | 54.708 | 1.00 | 38.60 | C |
| ATOM | 2587 | CG1 | VAL | R | 63 | 17.942 | 44.796 | 55.166 | 1.00 | 37.56 | C |
| ATOM | 2591 | CG2 | VAL | R | 63 | 16.409 | 44.743 | 53.186 | 1.00 | 36.99 | C |
| ATOM | 2595 | C | VAL | R | 63 | 15.911 | 43.238 | 56.726 | 1.00 | 38.44 | C |
| ATOM | 2596 | O | VAL | R | 63 | 15.432 | 44.146 | 57.424 | 1.00 | 35.43 | O |
| ATOM | 2598 | N | CYS | R | 64 | 16.453 | 42.122 | 57.218 | 1.00 | 39.13 | N |
| ATOM | 2599 | CA | CYS | R | 64 | 16.574 | 41.829 | 58.642 | 1.00 | 39.53 | C |
| ATOM | 2601 | CB | CYS | R | 64 | 15.975 | 40.462 | 58.962 | 1.00 | 37.79 | C |
| ATOM | 2604 | SG | CYS | R | 64 | 14.252 | 40.293 | 58.509 | 1.00 | 37.27 | S |
| ATOM | 2606 | C | CYS | R | 64 | 18.042 | 41.821 | 59.033 | 1.00 | 40.16 | C |
| ATOM | 2607 | O | CYS | R | 64 | 18.874 | 41.248 | 58.333 | 1.00 | 39.46 | O |
| ATOM | 2609 | N | ARG | R | 65 | 18.357 | 42.456 | 60.155 | 1.00 | 41.99 | N |
| ATOM | 2610 | CA | ARG | R | 65 | 19.727 | 42.558 | 60.605 | 1.00 | 42.37 | C |
| ATOM | 2612 | CB | ARG | R | 65 | 20.187 | 44.015 | 60.589 | 1.00 | 45.50 | C |
| ATOM | 2615 | CG | ARG | R | 65 | 21.606 | 44.201 | 61.124 | 1.00 | 49.03 | C |
| ATOM | 2618 | CD | ARG | R | 65 | 22.307 | 45.373 | 60.488 | 1.00 | 53.50 | C |
| ATOM | 2621 | NE | ARG | R | 65 | 21.671 | 46.643 | 60.830 | 1.00 | 58.11 | N |
| ATOM | 2623 | CZ | ARG | R | 65 | 22.051 | 47.824 | 60.345 | 1.00 | 59.54 | C |
| ATOM | 2624 | NH1 | ARG | R | 65 | 23.072 | 47.908 | 59.494 | 1.00 | 58.91 | N |
| ATOM | 2627 | NH2 | ARG | R | 65 | 21.406 | 48.927 | 60.712 | 1.00 | 60.05 | N |
| ATOM | 2630 | C | ARG | R | 65 | 19.857 | 41.966 | 62.002 | 1.00 | 39.31 | C |
| ATOM | 2631 | O | ARG | R | 65 | 19.013 | 42.229 | 62.867 | 1.00 | 37.03 | O |
| ATOM | 2633 | N | PRO | R | 66 | 20.895 | 41.138 | 62.220 | 1.00 | 35.57 | N |
| ATOM | 2634 | CA | PRO | R | 66 | 21.207 | 40.670 | 63.570 | 1.00 | 39.27 | C |
| ATOM | 2636 | CB | PRO | R | 66 | 22.576 | 40.000 | 63.406 | 1.00 | 36.92 | C |
| ATOM | 2639 | CG | PRO | R | 66 | 22.631 | 39.579 | 62.001 | 1.00 | 37.87 | C |
| ATOM | 2642 | CD | PRO | R | 66 | 21.805 | 40.560 | 61.214 | 1.00 | 37.99 | C |
| ATOM | 2645 | C | PRO | R | 66 | 21.287 | 41.811 | 64.586 | 1.00 | 37.84 | C |
| ATOM | 2646 | O | PRO | R | 66 | 21.848 | 42.866 | 64.293 | 1.00 | 37.06 | O |
| ATOM | 2647 | N | CYS | R | 67 | 20.709 | 41.601 | 65.762 | 1.00 | 40.43 | N |
| ATOM | 2648 | CA | CYS | R | 67 | 20.768 | 42.590 | 66.831 | 1.00 | 42.28 | C |
| ATOM | 2650 | CB | CYS | R | 67 | 19.914 | 42.148 | 68.022 | 1.00 | 41.98 | C |
| ATOM | 2653 | SG | CYS | R | 67 | 18.151 | 41.931 | 67.648 | 1.00 | 42.25 | S |
| ATOM | 2655 | C | CYS | R | 67 | 22.213 | 42.792 | 67.272 | 1.00 | 43.23 | C |
| ATOM | 2656 | O | CYS | R | 67 | 22.924 | 41.824 | 67.554 | 1.00 | 45.65 | O |
| ATOM | 2658 | N | GLY | R | 68 | 22.648 | 44.049 | 67.309 | 1.00 | 42.90 | N |
| ATOM | 2659 | CA | GLY | R | 68 | 24.007 | 44.377 | 67.724 | 1.00 | 43.36 | C |
| ATOM | 2662 | C | GLY | R | 68 | 24.134 | 44.378 | 69.236 | 1.00 | 43.50 | C |
| ATOM | 2663 | O | GLY | R | 68 | 23.136 | 44.240 | 69.937 | 1.00 | 44.94 | O |
| ATOM | 2665 | N | PRO | R | 69 | 25.365 | 44.553 | 69.750 | 1.00 | 45.11 | N |
| ATOM | 2666 | CA | PRO | R | 69 | 25.591 | 44.593 | 71.196 | 1.00 | 44.75 | C |
| ATOM | 2668 | CB | PRO | R | 69 | 27.060 | 45.024 | 71.311 | 1.00 | 45.27 | C |
| ATOM | 2671 | CG | PRO | R | 69 | 27.683 | 44.596 | 70.029 | 1.00 | 45.52 | C |
| ATOM | 2674 | CD | PRO | R | 69 | 26.620 | 44.739 | 68.990 | 1.00 | 45.92 | C |
| ATOM | 2677 | C | PRO | R | 69 | 24.677 | 45.598 | 71.902 | 1.00 | 45.05 | C |
| ATOM | 2678 | O | PRO | R | 69 | 24.506 | 46.724 | 71.427 | 1.00 | 44.17 | O |
| ATOM | 2679 | N | GLY | R | 70 | 24.085 | 45.177 | 73.016 | 1.00 | 44.10 | N |
| ATOM | 2680 | CA | GLY | R | 70 | 23.156 | 46.014 | 73.765 | 1.00 | 42.38 | C |
| ATOM | 2683 | C | GLY | R | 70 | 21.715 | 45.915 | 73.301 | 1.00 | 42.40 | C |
| ATOM | 2684 | O | GLY | R | 70 | 20.842 | 46.581 | 73.866 | 1.00 | 43.15 | O |
| ATOM | 2686 | N | PHE | R | 71 | 21.455 | 45.074 | 72.295 | 1.00 | 42.17 | N |
| ATOM | 2687 | CA | PHE | R | 71 | 20.109 | 44.915 | 71.726 | 1.00 | 40.60 | C |
| ATOM | 2689 | CB | PHE | R | 71 | 20.020 | 45.575 | 70.346 | 1.00 | 42.37 | C |
| ATOM | 2692 | CG | PHE | R | 71 | 20.181 | 47.059 | 70.384 | 1.00 | 42.12 | C |
| ATOM | 2693 | CD1 | PHE | R | 71 | 21.442 | 47.631 | 70.333 | 1.00 | 42.57 | C |
| ATOM | 2695 | CE1 | PHE | R | 71 | 21.599 | 49.006 | 70.378 | 1.00 | 43.91 | C |
| ATOM | 2697 | CZ | PHE | R | 71 | 20.480 | 49.827 | 70.483 | 1.00 | 44.13 | C |
| ATOM | 2699 | CE2 | PHE | R | 71 | 19.215 | 49.262 | 70.536 | 1.00 | 43.36 | C |
| ATOM | 2701 | CD2 | PHE | R | 71 | 19.071 | 47.886 | 70.488 | 1.00 | 42.20 | C |
| ATOM | 2703 | C | PHE | R | 71 | 19.715 | 43.455 | 71.594 | 1.00 | 37.90 | C |
| ATOM | 2704 | O | PHE | R | 71 | 20.569 | 42.582 | 71.480 | 1.00 | 34.21 | O |
| ATOM | 2706 | N | TYR | R | 72 | 18.407 | 43.209 | 71.589 | 1.00 | 39.91 | N |
| ATOM | 2707 | CA | TYR | R | 72 | 17.867 | 41.863 | 71.481 | 1.00 | 40.10 | C |
| ATOM | 2709 | CB | TYR | R | 72 | 17.738 | 41.224 | 72.874 | 1.00 | 38.83 | C |
| ATOM | 2712 | CG | TYR | R | 72 | 16.519 | 41.685 | 73.627 | 1.00 | 39.35 | C |
| ATOM | 2713 | CD1 | TYR | R | 72 | 15.357 | 40.926 | 73.636 | 1.00 | 36.50 | C |
| ATOM | 2715 | CE1 | TYR | R | 72 | 14.230 | 41.357 | 74.293 | 1.00 | 36.62 | C |
| ATOM | 2717 | CZ | TYR | R | 72 | 14.244 | 42.563 | 74.958 | 1.00 | 38.29 | C |
| ATOM | 2718 | OH | TYR | R | 72 | 13.119 | 42.979 | 75.632 | 1.00 | 41.35 | O |
| ATOM | 2720 | CE2 | TYR | R | 72 | 15.378 | 43.345 | 74.967 | 1.00 | 38.36 | C |
| ATOM | 2722 | CD2 | TYR | R | 72 | 16.509 | 42.908 | 74.294 | 1.00 | 40.79 | C |
| ATOM | 2724 | C | TYR | R | 72 | 16.501 | 41.887 | 70.796 | 1.00 | 40.05 | C |
| ATOM | 2725 | O | TYR | R | 72 | 15.868 | 42.929 | 70.681 | 1.00 | 39.45 | O |
| ATOM | 2727 | N | ASN | R | 73 | 16.064 | 40.721 | 70.338 | 1.00 | 42.29 | N |
| ATOM | 2728 | CA | ASN | R | 73 | 14.709 | 40.543 | 69.839 | 1.00 | 40.30 | C |
| ATOM | 2730 | CB | ASN | R | 73 | 14.601 | 40.917 | 68.353 | 1.00 | 42.10 | C |
| ATOM | 2733 | CG | ASN | R | 73 | 13.202 | 41.378 | 67.969 | 1.00 | 41.89 | C |
| ATOM | 2734 | OD1 | ASN | R | 73 | 12.225 | 41.007 | 68.611 | 1.00 | 44.85 | O |
| ATOM | 2735 | ND2 | ASN | R | 73 | 13.105 | 42.203 | 66.929 | 1.00 | 42.23 | N |
| ATOM | 2738 | C | ASN | R | 73 | 14.305 | 39.097 | 70.077 | 1.00 | 40.66 | C |
| ATOM | 2739 | O | ASN | R | 73 | 14.922 | 38.175 | 69.536 | 1.00 | 42.11 | O |
| ATOM | 2741 | N | ASP | R | 74 | 13.285 | 38.915 | 70.912 | 1.00 | 38.25 | N |
| ATOM | 2742 | CA | ASP | R | 74 | 12.869 | 37.591 | 71.388 | 1.00 | 38.21 | C |
| ATOM | 2744 | CB | ASP | R | 74 | 12.357 | 37.677 | 72.849 | 1.00 | 39.35 | C |
| ATOM | 2747 | CG | ASP | R | 74 | 11.301 | 38.777 | 73.062 | 1.00 | 39.21 | C |
| ATOM | 2748 | OD1 | ASP | R | 74 | 11.281 | 39.752 | 72.297 | 1.00 | 39.10 | O |
| ATOM | 2749 | OD2 | ASP | R | 74 | 10.488 | 38.669 | 74.002 | 1.00 | 41.38 | O |
| ATOM | 2750 | C | ASP | R | 74 | 11.814 | 36.909 | 70.509 | 1.00 | 37.47 | C |
| ATOM | 2751 | O | ASP | R | 74 | 11.521 | 35.736 | 70.709 | 1.00 | 40.58 | O |
| ATOM | 2753 | N | VAL | R | 75 | 11.261 | 37.634 | 69.539 | 1.00 | 36.04 | N |
| ATOM | 2754 | CA | VAL | R | 75 | 10.142 | 37.141 | 68.736 | 1.00 | 35.19 | C |
| ATOM | 2756 | CB | VAL | R | 75 | 8.791 | 37.773 | 69.170 | 1.00 | 34.59 | C |
| ATOM | 2758 | CG1 | VAL | R | 75 | 8.432 | 37.340 | 70.576 | 1.00 | 34.10 | C |
| ATOM | 2762 | CG2 | VAL | R | 75 | 8.828 | 39.308 | 69.065 | 1.00 | 34.70 | C |
| ATOM | 2766 | C | VAL | R | 75 | 10.313 | 37.398 | 67.241 | 1.00 | 37.15 | C |
| ATOM | 2767 | O | VAL | R | 75 | 11.150 | 38.199 | 66.813 | 1.00 | 39.08 | O |
| ATOM | 2769 | N | VAL | R | 76 | 9.500 | 36.684 | 66.467 | 1.00 | 39.52 | N |
| ATOM | 2770 | CA | VAL | R | 76 | 9.297 | 36.941 | 65.047 | 1.00 | 36.31 | C |
| ATOM | 2772 | CB | VAL | R | 76 | 8.362 | 35.876 | 64.423 | 1.00 | 33.40 | C |
| ATOM | 2774 | CG1 | VAL | R | 76 | 8.030 | 36.208 | 62.969 | 1.00 | 35.65 | C |
| ATOM | 2778 | CG2 | VAL | R | 76 | 8.980 | 34.484 | 64.534 | 1.00 | 30.29 | C |
| ATOM | 2782 | C | VAL | R | 76 | 8.642 | 38.313 | 64.973 | 1.00 | 40.52 | C |
| ATOM | 2783 | O | VAL | R | 76 | 7.681 | 38.576 | 65.700 | 1.00 | 40.07 | O |
| ATOM | 2785 | N | SER | R | 77 | 9.161 | 39.199 | 64.123 | 1.00 | 40.74 | N |
| ATOM | 2786 | CA | SER | R | 77 | 8.670 | 40.571 | 64.109 | 1.00 | 37.38 | C |
| ATOM | 2788 | CB | SER | R | 77 | 9.270 | 41.348 | 65.289 | 1.00 | 39.69 | C |
| ATOM | 2791 | OG | SER | R | 77 | 10.562 | 41.835 | 64.986 | 1.00 | 40.87 | O |
| ATOM | 2793 | C | SER | R | 77 | 8.946 | 41.337 | 62.819 | 1.00 | 37.15 | C |
| ATOM | 2794 | O | SER | R | 77 | 9.726 | 40.906 | 61.969 | 1.00 | 39.88 | O |
| ATOM | 2796 | N | SER | R | 78 | 8.279 | 42.484 | 62.702 | 1.00 | 33.97 | N |
| ATOM | 2797 | CA | SER | R | 78 | 8.552 | 43.470 | 61.675 | 1.00 | 35.67 | C |
| ATOM | 2799 | CB | SER | R | 78 | 7.309 | 43.689 | 60.810 | 1.00 | 35.00 | C |
| ATOM | 2802 | OG | SER | R | 78 | 6.189 | 44.071 | 61.590 | 1.00 | 36.65 | O |
| ATOM | 2804 | C | SER | R | 78 | 9.012 | 44.776 | 62.342 | 1.00 | 36.19 | C |
| ATOM | 2805 | O | SER | R | 78 | 8.732 | 45.867 | 61.859 | 1.00 | 38.90 | O |
| ATOM | 2807 | N | LYS | R | 79 | 9.753 | 44.636 | 63.439 | 1.00 | 38.16 | N |
| ATOM | 2808 | CA | LYS | R | 79 | 10.214 | 45.760 | 64.247 | 1.00 | 42.15 | C |
| ATOM | 2810 | CB | LYS | R | 79 | 9.505 | 45.742 | 65.608 | 1.00 | 41.48 | C |
| ATOM | 2813 | CG | LYS | R | 79 | 8.005 | 45.920 | 65.516 | 1.00 | 41.85 | C |
| ATOM | 2816 | CD | LYS | R | 79 | 7.421 | 46.437 | 66.820 | 1.00 | 42.48 | C |
| ATOM | 2819 | CE | LYS | R | 79 | 6.063 | 47.071 | 66.604 | 1.00 | 41.42 | C |
| ATOM | 2822 | NZ | LYS | R | 79 | 5.121 | 46.109 | 65.995 | 1.00 | 39.74 | N |
| ATOM | 2826 | C | LYS | R | 79 | 11.736 | 45.692 | 64.462 | 1.00 | 43.20 | C |
| ATOM | 2827 | O | LYS | R | 79 | 12.333 | 44.623 | 64.335 | 1.00 | 41.52 | O |
| ATOM | 2829 | N | PRO | R | 80 | 12.366 | 46.835 | 64.788 | 1.00 | 45.21 | N |
| ATOM | 2830 | CA | PRO | R | 80 | 13.787 | 46.812 | 65.140 | 1.00 | 45.44 | C |
| ATOM | 2832 | CB | PRO | R | 80 | 14.164 | 48.303 | 65.212 | 1.00 | 44.79 | C |
| ATOM | 2835 | CG | PRO | R | 80 | 12.897 | 49.017 | 65.452 | 1.00 | 45.90 | C |
| ATOM | 2838 | CD | PRO | R | 80 | 11.806 | 48.200 | 64.829 | 1.00 | 47.40 | C |
| ATOM | 2841 | C | PRO | R | 80 | 14.064 | 46.114 | 66.474 | 1.00 | 46.92 | C |
| ATOM | 2842 | O | PRO | R | 80 | 13.146 | 45.875 | 67.265 | 1.00 | 50.69 | O |
| ATOM | 2843 | N | CYS | R | 81 | 15.327 | 45.777 | 66.708 | 1.00 | 45.70 | N |
| ATOM | 2844 | CA | CYS | R | 81 | 15.733 | 45.164 | 67.968 | 1.00 | 45.17 | C |
| ATOM | 2846 | CB | CYS | R | 81 | 17.214 | 44.809 | 67.927 | 1.00 | 44.25 | C |
| ATOM | 2849 | SG | CYS | R | 81 | 17.635 | 43.633 | 66.631 | 1.00 | 42.68 | S |
| ATOM | 2851 | C | CYS | R | 81 | 15.451 | 46.081 | 69.159 | 1.00 | 43.72 | C |
| ATOM | 2852 | O | CYS | R | 81 | 15.483 | 47.302 | 69.032 | 1.00 | 42.70 | O |
| ATOM | 2854 | N | LYS | R | 82 | 15.162 | 45.468 | 70.304 | 1.00 | 46.15 | N |
| ATOM | 2855 | CA | LYS | R | 82 | 14.873 | 46.185 | 71.546 | 1.00 | 46.61 | C |
| ATOM | 2857 | CB | LYS | R | 82 | 13.909 | 45.363 | 72.409 | 1.00 | 47.58 | C |
| ATOM | 2860 | CG | LYS | R | 82 | 12.515 | 45.109 | 71.817 | 1.00 | 46.05 | C |
| ATOM | 2863 | CD | LYS | R | 82 | 12.001 | 43.745 | 72.291 | 1.00 | 46.55 | C |
| ATOM | 2866 | CE | LYS | R | 82 | 10.516 | 43.531 | 72.066 | 1.00 | 46.68 | C |
| ATOM | 2869 | NZ | LYS | R | 82 | 10.133 | 42.105 | 72.377 | 1.00 | 45.06 | N |
| ATOM | 2873 | C | LYS | R | 82 | 16.171 | 46.411 | 72.332 | 1.00 | 46.68 | C |
| ATOM | 2874 | O | LYS | R | 82 | 17.091 | 45.594 | 72.260 | 1.00 | 42.48 | O |
| ATOM | 2876 | N | PRO | R | 83 | 16.254 | 47.506 | 73.103 | 1.00 | 49.15 | N |
| ATOM | 2877 | CA | PRO | R | 83 | 17.467 | 47.685 | 73.908 | 1.00 | 47.77 | C |
| ATOM | 2879 | CB | PRO | R | 83 | 17.429 | 49.172 | 74.302 | 1.00 | 49.53 | C |
| ATOM | 2882 | CG | PRO | R | 83 | 16.221 | 49.757 | 73.599 | 1.00 | 52.66 | C |
| ATOM | 2885 | CD | PRO | R | 83 | 15.310 | 48.614 | 73.300 | 1.00 | 52.40 | C |
| ATOM | 2888 | C | PRO | R | 83 | 17.460 | 46.800 | 75.155 | 1.00 | 44.49 | C |
| ATOM | 2889 | O | PRO | R | 83 | 16.410 | 46.593 | 75.767 | 1.00 | 38.12 | O |
| ATOM | 2890 | N | CYS | R | 84 | 18.631 | 46.288 | 75.516 | 1.00 | 44.36 | N |
| ATOM | 2891 | CA | CYS | R | 84 | 18.766 | 45.441 | 76.692 | 1.00 | 44.02 | C |
| ATOM | 2893 | CB | CYS | R | 84 | 20.160 | 44.816 | 76.748 | 1.00 | 46.59 | C |
| ATOM | 2896 | SG | CYS | R | 84 | 20.503 | 43.692 | 75.365 | 1.00 | 48.61 | S |
| ATOM | 2898 | C | CYS | R | 84 | 18.492 | 46.234 | 77.960 | 1.00 | 40.25 | C |
| ATOM | 2899 | O | CYS | R | 84 | 18.765 | 47.432 | 78.036 | 1.00 | 38.27 | O |
| ATOM | 2901 | N | THR | R | 85 | 17.927 | 45.551 | 78.947 | 1.00 | 40.84 | N |
| ATOM | 2902 | CA | THR | R | 85 | 17.519 | 46.169 | 80.197 | 1.00 | 40.07 | C |
| ATOM | 2904 | CB | THR | R | 85 | 16.345 | 45.381 | 80.822 | 1.00 | 41.59 | C |
| ATOM | 2906 | OG1 | THR | R | 85 | 15.223 | 45.417 | 79.932 | 1.00 | 44.86 | O |
| ATOM | 2908 | CG2 | THR | R | 85 | 15.940 | 45.956 | 82.169 | 1.00 | 41.37 | C |
| ATOM | 2912 | C | THR | R | 85 | 18.680 | 46.166 | 81.171 | 1.00 | 39.20 | C |
| ATOM | 2913 | O | THR | R | 85 | 19.388 | 45.175 | 81.281 | 1.00 | 40.74 | O |
| ATOM | 2915 | N | TRP | R | 86 | 18.863 | 47.277 | 81.878 | 1.00 | 40.33 | N |
| ATOM | 2916 | CA | TRP | R | 86 | 19.834 | 47.364 | 82.966 | 1.00 | 37.17 | C |
| ATOM | 2918 | CB | TRP | R | 86 | 20.589 | 48.694 | 82.913 | 1.00 | 34.79 | C |
| ATOM | 2921 | CG | TRP | R | 86 | 21.508 | 48.798 | 81.750 | 1.00 | 33.82 | C |
| ATOM | 2922 | CD1 | TRP | R | 86 | 21.207 | 49.282 | 80.516 | 1.00 | 34.21 | C |
| ATOM | 2924 | NE1 | TRP | R | 86 | 22.313 | 49.219 | 79.697 | 1.00 | 34.87 | N |
| ATOM | 2926 | CE2 | TRP | R | 86 | 23.356 | 48.676 | 80.401 | 1.00 | 37.09 | C |
| ATOM | 2927 | CD2 | TRP | R | 86 | 22.884 | 48.394 | 81.702 | 1.00 | 33.40 | C |
| ATOM | 2928 | CE3 | TRP | R | 86 | 23.765 | 47.831 | 82.635 | 1.00 | 33.66 | C |
| ATOM | 2930 | CZ3 | TRP | R | 86 | 25.065 | 47.562 | 82.246 | 1.00 | 33.79 | C |
| ATOM | 2932 | CH2 | TRP | R | 86 | 25.510 | 47.854 | 80.939 | 1.00 | 34.60 | C |
| ATOM | 2934 | CZ2 | TRP | R | 86 | 24.674 | 48.411 | 80.006 | 1.00 | 35.25 | C |
| ATOM | 2936 | C | TRP | R | 86 | 19.132 | 47.223 | 84.303 | 1.00 | 37.98 | C |
| ATOM | 2937 | O | TRP | R | 86 | 18.145 | 47.915 | 84.578 | 1.00 | 39.84 | O |
| ATOM | 2939 | N | CYS | R | 87 | 19.643 | 46.318 | 85.132 | 1.00 | 41.68 | N |
| ATOM | 2940 | CA | CYS | R | 87 | 19.132 | 46.127 | 86.484 | 1.00 | 37.84 | C |
| ATOM | 2942 | CB | CYS | R | 87 | 19.639 | 44.803 | 87.066 | 1.00 | 36.52 | C |
| ATOM | 2945 | SG | CYS | R | 87 | 19.458 | 43.326 | 86.002 | 1.00 | 38.06 | S |
| ATOM | 2947 | C | CYS | R | 87 | 19.601 | 47.302 | 87.342 | 1.00 | 36.84 | C |
| ATOM | 2948 | O | CYS | R | 87 | 20.706 | 47.813 | 87.140 | 1.00 | 36.37 | O |
| ATOM | 2950 | N | ASN | R | 88 | 18.759 | 47.743 | 88.274 | 1.00 | 37.04 | N |
| ATOM | 2951 | CA | ASN | R | 88 | 19.134 | 48.795 | 89.225 | 1.00 | 37.77 | C |
| ATOM | 2953 | CB | ASN | R | 88 | 17.908 | 49.629 | 89.625 | 1.00 | 38.23 | C |
| ATOM | 2956 | CG | ASN | R | 88 | 18.278 | 50.949 | 90.311 | 1.00 | 37.84 | C |
| ATOM | 2957 | OD1 | ASN | R | 88 | 19.341 | 51.083 | 90.911 | 1.00 | 39.59 | O |
| ATOM | 2958 | ND2 | ASN | R | 88 | 17.386 | 51.925 | 90.223 | 1.00 | 37.33 | N |
| ATOM | 2961 | C | ASN | R | 88 | 19.807 | 48.165 | 90.447 | 1.00 | 39.72 | C |
| ATOM | 2962 | O | ASN | R | 88 | 19.165 | 47.889 | 91.460 | 1.00 | 40.55 | O |
| ATOM | 2964 | N | LEU | R | 89 | 21.114 | 47.949 | 90.331 | 1.00 | 41.13 | N |
| ATOM | 2965 | CA | LEU | R | 89 | 21.906 | 47.253 | 91.345 | 1.00 | 39.41 | C |
| ATOM | 2967 | CB | LEU | R | 89 | 23.370 | 47.177 | 90.903 | 1.00 | 40.73 | C |
| ATOM | 2970 | CG | LEU | R | 89 | 23.661 | 46.576 | 89.524 | 1.00 | 41.79 | C |
| ATOM | 2972 | CD1 | LEU | R | 89 | 25.159 | 46.615 | 89.245 | 1.00 | 40.59 | C |
| ATOM | 2976 | CD2 | LEU | R | 89 | 23.118 | 45.149 | 89.411 | 1.00 | 42.79 | C |
| ATOM | 2980 | C | LEU | R | 89 | 21.841 | 47.895 | 92.730 | 1.00 | 41.24 | C |
| ATOM | 2981 | O | LEU | R | 89 | 21.783 | 47.194 | 93.745 | 1.00 | 41.16 | O |
| ATOM | 2983 | N | ARG | R | 90 | 21.866 | 49.223 | 92.768 | 1.00 | 41.88 | N |
| ATOM | 2984 | CA | ARG | R | 90 | 21.842 | 49.970 | 94.029 | 1.00 | 40.28 | C |
| ATOM | 2986 | CB | ARG | R | 90 | 22.157 | 51.355 | 93.876 | 0.00 | 30.00 | C |
| ATOM | 2989 | CG | ARG | R | 90 | 22.292 | 52.046 | 95.234 | 0.00 | 30.00 | C |
| ATOM | 2992 | CD | ARG | R | 90 | 23.534 | 51.545 | 95.969 | 0.00 | 30.00 | C |
| ATOM | 2995 | NE | ARG | R | 90 | 23.664 | 52.208 | 97.274 | 0.00 | 30.00 | N |
| ATOM | 2997 | CZ | ARG | R | 90 | 24.681 | 51.939 | 98.115 | 0.00 | 30.00 | C |
| ATOM | 2998 | NH1 | ARG | R | 90 | 25.632 | 51.045 | 97.803 | 0.00 | 30.00 | N |
| ATOM | 3001 | NH2 | ARG | R | 90 | 24.745 | 52.580 | 99.296 | 0.00 | 30.00 | N |
| ATOM | 3004 | C | ARG | R | 90 | 20.605 | 49.634 | 94.858 | 1.00 | 39.42 | C |
| ATOM | 3005 | O | ARG | R | 90 | 20.684 | 49.511 | 96.081 | 1.00 | 42.72 | O |
| ATOM | 3007 | N | SER | R | 91 | 19.474 | 49.464 | 94.179 | 1.00 | 37.94 | N |
| ATOM | 3008 | CA | SER | R | 91 | 18.204 | 49.148 | 94.832 | 1.00 | 38.19 | C |
| ATOM | 3010 | CB | SER | R | 91 | 17.049 | 49.421 | 93.863 | 1.00 | 35.54 | C |
| ATOM | 3013 | OG | SER | R | 91 | 17.054 | 48.532 | 92.762 | 1.00 | 35.81 | O |
| ATOM | 3015 | C | SER | R | 91 | 18.103 | 47.708 | 95.396 | 1.00 | 39.30 | C |
| ATOM | 3016 | O | SER | R | 91 | 17.198 | 47.418 | 96.179 | 1.00 | 37.67 | O |
| ATOM | 3018 | N | GLY | R | 92 | 19.038 | 46.832 | 95.019 | 1.00 | 39.53 | N |
| ATOM | 3019 | CA | GLY | R | 92 | 19.028 | 45.419 | 95.434 | 1.00 | 38.40 | C |
| ATOM | 3022 | C | GLY | R | 92 | 18.712 | 44.451 | 94.294 | 1.00 | 38.13 | C |
| ATOM | 3023 | O | GLY | R | 92 | 18.579 | 43.242 | 94.517 | 1.00 | 39.48 | O |
| ATOM | 3025 | N | SER | R | 93 | 18.592 | 44.983 | 93.078 | 1.00 | 34.70 | N |
| ATOM | 3026 | CA | SER | R | 93 | 18.355 | 44.186 | 91.871 | 1.00 | 39.05 | C |
| ATOM | 3028 | CB | SER | R | 93 | 17.880 | 45.088 | 90.716 | 1.00 | 40.03 | C |
| ATOM | 3031 | OG | SER | R | 93 | 17.480 | 44.340 | 89.579 | 1.00 | 39.62 | O |
| ATOM | 3033 | C | SER | R | 93 | 19.640 | 43.482 | 91.467 | 1.00 | 38.65 | C |
| ATOM | 3034 | O | SER | R | 93 | 20.710 | 44.077 | 91.474 | 1.00 | 37.25 | O |
| ATOM | 3036 | N | GLU | R | 94 | 19.524 | 42.212 | 91.108 | 1.00 | 42.11 | N |
| ATOM | 3037 | CA | GLU | R | 94 | 20.674 | 41.405 | 90.726 | 1.00 | 42.41 | C |
| ATOM | 3039 | CB | GLU | R | 94 | 20.764 | 40.214 | 91.674 | 1.00 | 45.66 | C |
| ATOM | 3042 | CG | GLU | R | 94 | 21.798 | 39.157 | 91.322 | 1.00 | 48.13 | C |
| ATOM | 3045 | CD | GLU | R | 94 | 21.755 | 37.984 | 92.285 | 1.00 | 51.61 | C |
| ATOM | 3046 | OE1 | GLU | R | 94 | 21.047 | 38.094 | 93.315 | 1.00 | 54.93 | O |
| ATOM | 3047 | OE2 | GLU | R | 94 | 22.422 | 36.960 | 92.010 | 1.00 | 53.86 | O |
| ATOM | 3048 | C | GLU | R | 94 | 20.522 | 40.935 | 89.286 | 1.00 | 38.20 | C |
| ATOM | 3049 | O | GLU | R | 94 | 19.449 | 40.483 | 88.891 | 1.00 | 33.53 | O |
| ATOM | 3051 | N | ARG | R | 95 | 21.591 | 41.039 | 88.502 | 1.00 | 38.29 | N |
| ATOM | 3052 | CA | ARG | R | 95 | 21.564 | 40.549 | 87.131 | 1.00 | 39.34 | C |
| ATOM | 3054 | CB | ARG | R | 95 | 22.578 | 41.266 | 86.248 | 1.00 | 39.19 | C |
| ATOM | 3057 | CG | ARG | R | 95 | 22.535 | 40.782 | 84.795 | 1.00 | 40.92 | C |
| ATOM | 3060 | CD | ARG | R | 95 | 23.322 | 41.684 | 83.869 | 1.00 | 41.14 | C |
| ATOM | 3063 | NE | ARG | R | 95 | 24.733 | 41.735 | 84.242 | 1.00 | 41.41 | N |
| ATOM | 3065 | CZ | ARG | R | 95 | 25.646 | 42.496 | 83.643 | 1.00 | 43.25 | C |
| ATOM | 3066 | NH1 | ARG | R | 95 | 25.316 | 43.283 | 82.617 | 1.00 | 45.91 | N |
| ATOM | 3069 | NH2 | ARG | R | 95 | 26.901 | 42.462 | 84.065 | 1.00 | 41.35 | N |
| ATOM | 3072 | C | ARG | R | 95 | 21.850 | 39.063 | 87.131 | 1.00 | 39.90 | C |
| ATOM | 3073 | O | ARG | R | 95 | 22.992 | 38.645 | 87.330 | 1.00 | 41.04 | O |
| ATOM | 3075 | N | LYS | R | 96 | 20.810 | 38.270 | 86.899 | 1.00 | 40.23 | N |
| ATOM | 3076 | CA | LYS | R | 96 | 20.924 | 36.815 | 86.923 | 1.00 | 41.08 | C |
| ATOM | 3078 | CB | LYS | R | 96 | 19.590 | 36.195 | 87.342 | 1.00 | 40.24 | C |
| ATOM | 3081 | CG | LYS | R | 96 | 19.157 | 36.575 | 88.754 | 1.00 | 40.56 | C |
| ATOM | 3084 | CD | LYS | R | 96 | 20.021 | 35.928 | 89.844 | 1.00 | 40.66 | C |
| ATOM | 3087 | CE | LYS | R | 96 | 19.837 | 34.409 | 89.889 | 1.00 | 42.22 | C |
| ATOM | 3090 | NZ | LYS | R | 96 | 20.235 | 33.829 | 91.212 | 1.00 | 42.40 | N |
| ATOM | 3094 | C | LYS | R | 96 | 21.378 | 36.242 | 85.583 | 1.00 | 41.82 | C |
| ATOM | 3095 | O | LYS | R | 96 | 21.974 | 35.175 | 85.542 | 1.00 | 40.66 | O |
| ATOM | 3097 | N | GLN | R | 97 | 21.085 | 36.943 | 84.488 | 1.00 | 45.91 | N |
| ATOM | 3098 | CA | GLN | R | 97 | 21.542 | 36.531 | 83.159 | 1.00 | 46.83 | C |
| ATOM | 3100 | CB | GLN | R | 97 | 20.480 | 35.700 | 82.438 | 1.00 | 50.23 | C |
| ATOM | 3103 | CG | GLN | R | 97 | 20.107 | 34.413 | 83.121 | 1.00 | 55.48 | C |
| ATOM | 3106 | CD | GLN | R | 97 | 19.104 | 33.609 | 82.323 | 1.00 | 57.47 | C |
| ATOM | 3107 | OE1 | GLN | R | 97 | 19.115 | 33.630 | 81.088 | 1.00 | 60.38 | O |
| ATOM | 3108 | NE2 | GLN | R | 97 | 18.232 | 32.880 | 83.027 | 1.00 | 61.22 | N |
| ATOM | 3111 | C | GLN | R | 97 | 21.859 | 37.733 | 82.286 | 1.00 | 43.79 | C |
| ATOM | 3112 | O | GLN | R | 97 | 21.193 | 38.758 | 82.366 | 1.00 | 39.90 | O |
| ATOM | 3114 | N | LEU | R | 98 | 22.863 | 37.571 | 81.431 | 1.00 | 42.86 | N |
| ATOM | 3115 | CA | LEU | R | 98 | 23.208 | 38.563 | 80.436 | 1.00 | 42.17 | C |
| ATOM | 3117 | CB | LEU | R | 98 | 24.602 | 38.280 | 79.863 | 1.00 | 42.73 | C |
| ATOM | 3120 | CG | LEU | R | 98 | 25.797 | 38.426 | 80.811 | 1.00 | 43.29 | C |
| ATOM | 3122 | CD1 | LEU | R | 98 | 27.077 | 38.025 | 80.096 | 1.00 | 43.48 | C |
| ATOM | 3126 | CD2 | LEU | R | 98 | 25.902 | 39.846 | 81.343 | 1.00 | 43.89 | C |
| ATOM | 3130 | C | LEU | R | 98 | 22.183 | 38.572 | 79.307 | 1.00 | 41.40 | C |
| ATOM | 3131 | O | LEU | R | 98 | 21.573 | 37.555 | 78.981 | 1.00 | 42.32 | O |
| ATOM | 3133 | N | CYS | R | 99 | 21.991 | 39.748 | 78.732 | 1.00 | 42.90 | N |
| ATOM | 3134 | CA | CYS | R | 99 | 21.200 | 39.915 | 77.531 | 1.00 | 41.33 | C |
| ATOM | 3136 | CB | CYS | R | 99 | 21.099 | 41.405 | 77.192 | 1.00 | 43.88 | C |
| ATOM | 3139 | SG | CYS | R | 99 | 19.831 | 41.900 | 76.004 | 1.00 | 46.86 | S |
| ATOM | 3141 | C | CYS | R | 99 | 21.936 | 39.192 | 76.423 | 1.00 | 40.18 | C |
| ATOM | 3142 | O | CYS | R | 99 | 23.168 | 39.224 | 76.373 | 1.00 | 38.68 | O |
| ATOM | 3144 | N | THR | R | 100 | 21.185 | 38.507 | 75.567 | 1.00 | 39.85 | N |
| ATOM | 3145 | CA | THR | R | 100 | 21.717 | 37.978 | 74.321 | 1.00 | 41.13 | C |
| ATOM | 3147 | CB | THR | R | 100 | 21.639 | 36.446 | 74.287 | 1.00 | 40.68 | C |
| ATOM | 3149 | OG1 | THR | R | 100 | 20.269 | 36.046 | 74.265 | 1.00 | 38.28 | O |
| ATOM | 3151 | CG2 | THR | R | 100 | 22.326 | 35.845 | 75.502 | 1.00 | 39.23 | C |
| ATOM | 3155 | C | THR | R | 100 | 20.910 | 38.555 | 73.156 | 1.00 | 40.69 | C |
| ATOM | 3156 | O | THR | R | 100 | 19.959 | 39.303 | 73.362 | 1.00 | 38.84 | O |
| ATOM | 3158 | N | ALA | R | 101 | 21.287 | 38.201 | 71.935 | 1.00 | 40.91 | N |
| ATOM | 3159 | CA | ALA | R | 101 | 20.567 | 38.653 | 70.750 | 1.00 | 41.08 | C |
| ATOM | 3161 | CB | ALA | R | 101 | 21.245 | 38.108 | 69.482 | 1.00 | 38.94 | C |
| ATOM | 3165 | C | ALA | R | 101 | 19.079 | 38.268 | 70.775 | 1.00 | 40.12 | C |
| ATOM | 3166 | O | ALA | R | 101 | 18.261 | 38.955 | 70.180 | 1.00 | 42.14 | O |
| ATOM | 3168 | N | THR | R | 102 | 18.733 | 37.189 | 71.475 | 1.00 | 41.49 | N |
| ATOM | 3169 | CA | THR | R | 102 | 17.372 | 36.650 | 71.464 | 1.00 | 43.28 | C |
| ATOM | 3171 | CB | THR | R | 102 | 17.384 | 35.158 | 71.096 | 1.00 | 42.17 | C |
| ATOM | 3173 | OG1 | THR | R | 102 | 18.099 | 34.431 | 72.100 | 1.00 | 44.61 | O |
| ATOM | 3175 | CG2 | THR | R | 102 | 18.049 | 34.943 | 69.736 | 1.00 | 40.58 | C |
| ATOM | 3179 | C | THR | R | 102 | 16.587 | 36.809 | 72.779 | 1.00 | 44.45 | C |
| ATOM | 3180 | O | THR | R | 102 | 15.405 | 36.456 | 72.834 | 1.00 | 42.84 | O |
| ATOM | 3182 | N | GLN | R | 103 | 17.223 | 37.322 | 73.832 | 1.00 | 45.06 | N |
| ATOM | 3183 | CA | GLN | R | 103 | 16.511 | 37.571 | 75.095 | 1.00 | 46.85 | C |
| ATOM | 3185 | CB | GLN | R | 103 | 16.426 | 36.294 | 75.937 | 1.00 | 48.63 | C |
| ATOM | 3188 | CG | GLN | R | 103 | 17.758 | 35.661 | 76.194 | 1.00 | 52.01 | C |
| ATOM | 3191 | CD | GLN | R | 103 | 17.647 | 34.242 | 76.709 | 1.00 | 53.48 | C |
| ATOM | 3192 | OE1 | GLN | R | 103 | 16.977 | 33.987 | 77.712 | 1.00 | 56.52 | O |
| ATOM | 3193 | NE2 | GLN | R | 103 | 18.322 | 33.311 | 76.033 | 1.00 | 52.37 | N |
| ATOM | 3196 | C | GLN | R | 103 | 17.106 | 38.703 | 75.928 | 1.00 | 44.63 | C |
| ATOM | 3197 | O | GLN | R | 103 | 18.293 | 39.006 | 75.843 | 1.00 | 49.16 | O |
| ATOM | 3199 | N | ASP | R | 104 | 16.251 | 39.318 | 76.735 | 1.00 | 40.34 | N |
| ATOM | 3200 | CA | ASP | R | 104 | 16.640 | 40.436 | 77.569 | 1.00 | 41.70 | C |
| ATOM | 3202 | CB | ASP | R | 104 | 15.391 | 41.205 | 78.034 | 1.00 | 40.47 | C |
| ATOM | 3205 | CG | ASP | R | 104 | 15.689 | 42.645 | 78.441 | 1.00 | 41.52 | C |
| ATOM | 3206 | OD1 | ASP | R | 104 | 16.836 | 43.119 | 78.290 | 1.00 | 39.21 | O |
| ATOM | 3207 | OD2 | ASP | R | 104 | 14.754 | 43.313 | 78.915 | 1.00 | 40.42 | O |
| ATOM | 3208 | C | ASP | R | 104 | 17.426 | 39.951 | 78.789 | 1.00 | 41.49 | C |
| ATOM | 3209 | O | ASP | R | 104 | 17.373 | 38.779 | 79.176 | 1.00 | 40.19 | O |
| ATOM | 3211 | N | THR | R | 105 | 18.167 | 40.883 | 79.375 | 1.00 | 42.77 | N |
| ATOM | 3212 | CA | THR | R | 105 | 18.724 | 40.742 | 80.711 | 1.00 | 40.70 | C |
| ATOM | 3214 | CB | THR | R | 105 | 19.139 | 42.127 | 81.222 | 1.00 | 38.83 | C |
| ATOM | 3216 | OG1 | THR | R | 105 | 19.813 | 42.831 | 80.164 | 1.00 | 33.82 | O |
| ATOM | 3218 | CG2 | THR | R | 105 | 20.033 | 42.025 | 82.446 | 1.00 | 38.20 | C |
| ATOM | 3222 | C | THR | R | 105 | 17.656 | 40.169 | 81.635 | 1.00 | 41.30 | C |
| ATOM | 3223 | O | THR | R | 105 | 16.480 | 40.478 | 81.478 | 1.00 | 41.61 | O |
| ATOM | 3225 | N | VAL | R | 106 | 18.054 | 39.314 | 82.573 | 1.00 | 42.93 | N |
| ATOM | 3226 | CA | VAL | R | 106 | 17.146 | 38.854 | 83.623 | 1.00 | 40.98 | C |
| ATOM | 3228 | CB | VAL | R | 106 | 17.104 | 37.312 | 83.756 | 1.00 | 41.63 | C |
| ATOM | 3230 | CG1 | VAL | R | 106 | 16.229 | 36.902 | 84.936 | 1.00 | 42.13 | C |
| ATOM | 3234 | CG2 | VAL | R | 106 | 16.577 | 36.674 | 82.471 | 1.00 | 41.41 | C |
| ATOM | 3238 | C | VAL | R | 106 | 17.605 | 39.502 | 84.924 | 1.00 | 41.13 | C |
| ATOM | 3239 | O | VAL | R | 106 | 18.742 | 39.310 | 85.346 | 1.00 | 38.65 | O |
| ATOM | 3241 | N | CYS | R | 107 | 16.713 | 40.300 | 85.514 | 1.00 | 43.12 | N |
| ATOM | 3242 | CA | CYS | R | 107 | 16.957 | 41.042 | 86.745 | 1.00 | 37.37 | C |
| ATOM | 3244 | CB | CYS | R | 107 | 16.609 | 42.515 | 86.551 | 1.00 | 38.65 | C |
| ATOM | 3247 | SG | CYS | R | 107 | 17.545 | 43.356 | 85.281 | 1.00 | 38.44 | S |
| ATOM | 3249 | C | CYS | R | 107 | 16.049 | 40.474 | 87.813 | 1.00 | 36.44 | C |
| ATOM | 3250 | O | CYS | R | 107 | 14.868 | 40.263 | 87.557 | 1.00 | 37.02 | O |
| ATOM | 3252 | N | ARG | R | 108 | 16.589 | 40.232 | 89.006 | 1.00 | 36.58 | N |
| ATOM | 3253 | CA | ARG | R | 108 | 15.811 | 39.681 | 90.104 | 1.00 | 35.09 | C |
| ATOM | 3255 | CB | ARG | R | 108 | 16.038 | 38.173 | 90.210 | 1.00 | 35.87 | C |
| ATOM | 3258 | CG | ARG | R | 108 | 15.418 | 37.360 | 89.072 | 1.00 | 36.29 | C |
| ATOM | 3261 | CD | ARG | R | 108 | 13.894 | 37.296 | 89.175 | 1.00 | 35.60 | C |
| ATOM | 3264 | NE | ARG | R | 108 | 13.317 | 36.495 | 88.094 | 1.00 | 32.85 | N |
| ATOM | 3266 | CZ | ARG | R | 108 | 12.989 | 36.961 | 86.892 | 1.00 | 33.36 | C |
| ATOM | 3267 | NH1 | ARG | R | 108 | 13.158 | 38.239 | 86.584 | 1.00 | 32.00 | N |
| ATOM | 3270 | NH2 | ARG | R | 108 | 12.484 | 36.139 | 85.982 | 1.00 | 35.99 | N |
| ATOM | 3273 | C | ARG | R | 108 | 16.172 | 40.366 | 91.409 | 1.00 | 36.22 | C |
| ATOM | 3274 | O | ARG | R | 108 | 17.340 | 40.626 | 91.676 | 1.00 | 38.57 | O |
| ATOM | 3276 | N | CYS | R | 109 | 15.163 | 40.668 | 92.221 | 1.00 | 38.83 | N |
| ATOM | 3277 | CA | CYS | R | 109 | 15.389 | 41.334 | 93.501 | 1.00 | 38.85 | C |
| ATOM | 3279 | CB | CYS | R | 109 | 14.102 | 41.976 | 94.006 | 1.00 | 38.93 | C |
| ATOM | 3282 | SG | CYS | R | 109 | 13.525 | 43.340 | 92.990 | 1.00 | 40.16 | S |
| ATOM | 3284 | C | CYS | R | 109 | 15.935 | 40.350 | 94.533 | 1.00 | 36.92 | C |
| ATOM | 3285 | O | CYS | R | 109 | 15.488 | 39.213 | 94.608 | 1.00 | 38.83 | O |
| ATOM | 3287 | N | ARG | R | 110 | 16.906 | 40.800 | 95.319 | 1.00 | 40.22 | N |
| ATOM | 3288 | CA | ARG | R | 110 | 17.593 | 39.936 | 96.283 | 1.00 | 41.24 | C |
| ATOM | 3290 | CB | ARG | R | 110 | 18.930 | 40.553 | 96.704 | 1.00 | 43.64 | C |
| ATOM | 3293 | CG | ARG | R | 110 | 19.979 | 40.567 | 95.603 | 1.00 | 46.93 | C |
| ATOM | 3296 | CD | ARG | R | 110 | 21.185 | 41.406 | 96.013 | 1.00 | 48.16 | C |
| ATOM | 3299 | NE | ARG | R | 110 | 22.151 | 41.592 | 94.925 | 1.00 | 48.54 | N |
| ATOM | 3301 | CZ | ARG | R | 110 | 23.066 | 40.696 | 94.547 | 1.00 | 51.36 | C |
| ATOM | 3302 | NH1 | ARG | R | 110 | 23.160 | 39.508 | 95.145 | 1.00 | 51.80 | N |
| ATOM | 3305 | NH2 | ARG | R | 110 | 23.899 | 40.987 | 93.554 | 1.00 | 51.84 | N |
| ATOM | 3308 | C | ARG | R | 110 | 16.749 | 39.676 | 97.523 | 1.00 | 39.27 | C |
| ATOM | 3309 | O | ARG | R | 110 | 15.732 | 40.332 | 97.754 | 1.00 | 39.00 | O |
| ATOM | 3311 | N | ALA | R | 111 | 17.188 | 38.707 | 98.318 | 1.00 | 38.68 | N |
| ATOM | 3312 | CA | ALA | R | 111 | 16.599 | 38.453 | 99.621 | 1.00 | 38.43 | C |
| ATOM | 3314 | CB | ALA | R | 111 | 17.378 | 37.359 | 100.348 | 1.00 | 39.35 | C |
| ATOM | 3318 | C | ALA | R | 111 | 16.602 | 39.739 | 100.435 | 1.00 | 37.64 | C |
| ATOM | 3319 | O | ALA | R | 111 | 17.577 | 40.498 | 100.400 | 1.00 | 39.53 | O |
| ATOM | 3321 | N | GLY | R | 112 | 15.503 | 39.987 | 101.146 | 1.00 | 35.96 | N |
| ATOM | 3322 | CA | GLY | R | 112 | 15.348 | 41.196 | 101.952 | 1.00 | 37.19 | C |
| ATOM | 3325 | C | GLY | R | 112 | 14.764 | 42.373 | 101.197 | 1.00 | 38.74 | C |
| ATOM | 3326 | O | GLY | R | 112 | 14.507 | 43.424 | 101.784 | 1.00 | 40.74 | O |
| ATOM | 3328 | N | THR | R | 113 | 14.568 | 42.209 | 99.891 | 1.00 | 41.61 | N |
| ATOM | 3329 | CA | THR | R | 113 | 13.972 | 43.247 | 99.062 | 1.00 | 40.63 | C |
| ATOM | 3331 | CB | THR | R | 113 | 15.011 | 43.897 | 98.105 | 1.00 | 39.77 | C |
| ATOM | 3333 | OG1 | THR | R | 113 | 15.276 | 43.027 | 96.997 | 1.00 | 36.97 | O |
| ATOM | 3335 | CG2 | THR | R | 113 | 16.313 | 44.197 | 98.836 | 1.00 | 40.04 | C |
| ATOM | 3339 | C | THR | R | 113 | 12.836 | 42.646 | 98.244 | 1.00 | 41.92 | C |
| ATOM | 3340 | O | THR | R | 113 | 12.714 | 41.423 | 98.136 | 1.00 | 39.87 | O |
| ATOM | 3342 | N | GLN | R | 114 | 12.004 | 43.522 | 97.688 | 1.00 | 42.78 | N |
| ATOM | 3343 | CA | GLN | R | 114 | 10.927 | 43.126 | 96.789 | 1.00 | 41.54 | C |
| ATOM | 3345 | CB | GLN | R | 114 | 9.579 | 43.144 | 97.518 | 1.00 | 41.16 | C |
| ATOM | 3348 | CG | GLN | R | 114 | 9.171 | 44.517 | 98.056 | 1.00 | 41.55 | C |
| ATOM | 3351 | CD | GLN | R | 114 | 7.796 | 44.526 | 98.725 | 1.00 | 42.44 | C |
| ATOM | 3352 | OE1 | GLN | R | 114 | 7.060 | 43.538 | 98.689 | 1.00 | 41.58 | O |
| ATOM | 3353 | NE2 | GLN | R | 114 | 7.450 | 45.655 | 99.338 | 1.00 | 39.24 | N |
| ATOM | 3356 | C | GLN | R | 114 | 10.874 | 44.079 | 95.597 | 1.00 | 41.33 | C |
| ATOM | 3357 | O | GLN | R | 114 | 11.330 | 45.228 | 95.694 | 1.00 | 38.51 | O |
| ATOM | 3359 | N | PRO | R | 115 | 10.295 | 43.617 | 94.473 | 1.00 | 42.37 | N |
| ATOM | 3360 | CA | PRO | R | 115 | 10.124 | 44.496 | 93.311 | 1.00 | 42.04 | C |
| ATOM | 3362 | CB | PRO | R | 115 | 9.512 | 43.574 | 92.249 | 1.00 | 39.19 | C |
| ATOM | 3365 | CG | PRO | R | 115 | 9.654 | 42.211 | 92.747 | 1.00 | 39.88 | C |
| ATOM | 3368 | CD | PRO | R | 115 | 9.768 | 42.264 | 94.225 | 1.00 | 39.73 | C |
| ATOM | 3371 | C | PRO | R | 115 | 9.178 | 45.672 | 93.566 | 1.00 | 43.79 | C |
| ATOM | 3372 | O | PRO | R | 115 | 8.096 | 45.482 | 94.118 | 1.00 | 44.59 | O |
| ATOM | 3373 | N | LEU | R | 116 | 9.596 | 46.872 | 93.170 | 1.00 | 45.69 | N |
| ATOM | 3374 | CA | LEU | R | 116 | 8.696 | 48.021 | 93.096 | 1.00 | 47.91 | C |
| ATOM | 3376 | CB | LEU | R | 116 | 9.474 | 49.330 | 93.267 | 1.00 | 49.98 | C |
| ATOM | 3379 | CG | LEU | R | 116 | 10.102 | 49.611 | 94.636 | 1.00 | 50.77 | C |
| ATOM | 3381 | CD1 | LEU | R | 116 | 11.060 | 50.801 | 94.540 | 1.00 | 50.97 | C |
| ATOM | 3385 | CD2 | LEU | R | 116 | 9.019 | 49.859 | 95.684 | 1.00 | 51.64 | C |
| ATOM | 3389 | C | LEU | R | 116 | 8.011 | 48.014 | 91.734 | 1.00 | 49.03 | C |
| ATOM | 3390 | O | LEU | R | 116 | 8.396 | 47.250 | 90.852 | 1.00 | 50.67 | O |
| ATOM | 3392 | N | ASP | R | 117 | 6.990 | 48.852 | 91.565 | 1.00 | 51.13 | N |
| ATOM | 3393 | CA | ASP | R | 117 | 6.452 | 49.125 | 90.230 | 1.00 | 51.41 | C |
| ATOM | 3395 | CB | ASP | R | 117 | 5.222 | 50.037 | 90.278 | 1.00 | 53.66 | C |
| ATOM | 3398 | CG | ASP | R | 117 | 3.976 | 49.318 | 90.774 | 1.00 | 56.60 | C |
| ATOM | 3399 | OD1 | ASP | R | 117 | 4.101 | 48.212 | 91.347 | 1.00 | 57.65 | O |
| ATOM | 3400 | OD2 | ASP | R | 117 | 2.866 | 49.869 | 90.596 | 1.00 | 59.57 | O |
| ATOM | 3401 | C | ASP | R | 117 | 7.575 | 49.778 | 89.440 | 1.00 | 52.66 | C |
| ATOM | 3402 | O | ASP | R | 117 | 8.174 | 50.758 | 89.887 | 1.00 | 55.22 | O |
| ATOM | 3404 | N | SER | R | 118 | 7.869 | 49.213 | 88.275 | 1.00 | 51.03 | N |
| ATOM | 3405 | CA | SER | R | 118 | 9.129 | 49.474 | 87.600 | 1.00 | 46.65 | C |
| ATOM | 3407 | CB | SER | R | 118 | 10.174 | 48.480 | 88.116 | 1.00 | 46.26 | C |
| ATOM | 3410 | OG | SER | R | 118 | 11.447 | 48.708 | 87.546 | 1.00 | 47.94 | O |
| ATOM | 3412 | C | SER | R | 118 | 8.983 | 49.342 | 86.091 | 1.00 | 45.14 | C |
| ATOM | 3551 | O | CYS | R | 128 | 11.804 | 46.143 | 104.955 | 1.00 | 41.17 | O |
| ATOM | 3553 | N | PRO | R | 129 | 9.614 | 46.350 | 104.385 | 1.00 | 41.88 | N |
| ATOM | 3554 | CA | PRO | R | 129 | 9.280 | 47.143 | 105.567 | 1.00 | 40.30 | C |
| ATOM | 3556 | CB | PRO | R | 129 | 7.758 | 47.296 | 105.472 | 1.00 | 40.62 | C |
| ATOM | 3559 | CG | PRO | R | 129 | 7.430 | 47.108 | 104.048 | 1.00 | 40.80 | C |
| ATOM | 3562 | CD | PRO | R | 129 | 8.457 | 46.188 | 103.483 | 1.00 | 40.98 | C |
| ATOM | 3565 | C | PRO | R | 129 | 9.647 | 46.427 | 106.872 | 1.00 | 40.12 | C |
| ATOM | 3566 | O | PRO | R | 129 | 9.878 | 45.210 | 106.864 | 1.00 | 38.40 | O |
| ATOM | 3567 | N | PRO | R | 130 | 9.685 | 47.170 | 107.994 | 1.00 | 38.10 | N |
| ATOM | 3568 | CA | PRO | R | 130 | 9.993 | 46.551 | 109.282 | 1.00 | 38.42 | C |
| ATOM | 3570 | CB | PRO | R | 130 | 9.680 | 47.662 | 110.287 | 1.00 | 38.90 | C |
| ATOM | 3573 | CG | PRO | R | 130 | 9.841 | 48.919 | 109.518 | 1.00 | 38.43 | C |
| ATOM | 3576 | CD | PRO | R | 130 | 9.420 | 48.613 | 108.123 | 1.00 | 37.59 | C |
| ATOM | 3579 | C | PRO | R | 130 | 9.115 | 45.332 | 109.556 | 1.00 | 36.74 | C |
| ATOM | 3580 | O | PRO | R | 130 | 7.926 | 45.345 | 109.230 | 1.00 | 37.44 | O |
| ATOM | 3581 | N | GLY | R | 131 | 9.699 | 44.286 | 110.128 | 1.00 | 35.10 | N |
| ATOM | 3582 | CA | GLY | R | 131 | 8.946 | 43.080 | 110.461 | 1.00 | 35.95 | C |
| ATOM | 3585 | C | GLY | R | 131 | 8.398 | 42.299 | 109.274 | 1.00 | 37.04 | C |
| ATOM | 3586 | O | GLY | R | 131 | 7.451 | 41.524 | 109.428 | 1.00 | 37.03 | O |
| ATOM | 3588 | N | HIS | R | 132 | 8.985 | 42.502 | 108.091 | 1.00 | 37.84 | N |
| ATOM | 3589 | CA | HIS | R | 132 | 8.619 | 41.751 | 106.884 | 1.00 | 37.23 | C |
| ATOM | 3591 | CB | HIS | R | 132 | 8.063 | 42.679 | 105.795 | 1.00 | 35.37 | C |
| ATOM | 3594 | CG | HIS | R | 132 | 6.665 | 43.145 | 106.053 | 1.00 | 34.92 | C |
| ATOM | 3595 | ND1 | HIS | R | 132 | 6.337 | 43.972 | 107.106 | 1.00 | 37.51 | N |
| ATOM | 3597 | CE1 | HIS | R | 132 | 5.038 | 44.215 | 107.084 | 1.00 | 37.56 | C |
| ATOM | 3599 | NE2 | HIS | R | 132 | 4.513 | 43.576 | 106.053 | 1.00 | 37.50 | N |
| ATOM | 3601 | CD2 | HIS | R | 132 | 5.510 | 42.903 | 105.390 | 1.00 | 34.76 | C |
| ATOM | 3603 | C | HIS | R | 132 | 9.835 | 41.009 | 106.352 | 1.00 | 38.32 | C |
| ATOM | 3604 | O | HIS | R | 132 | 10.972 | 41.410 | 106.606 | 1.00 | 39.01 | O |
| ATOM | 3606 | N | PHE | R | 133 | 9.588 | 39.924 | 105.622 | 1.00 | 39.09 | N |
| ATOM | 3607 | CA | PHE | R | 133 | 10.663 | 39.142 | 105.008 | 1.00 | 37.71 | C |
| ATOM | 3609 | CB | PHE | R | 133 | 10.876 | 37.817 | 105.761 | 1.00 | 35.63 | C |
| ATOM | 3612 | CG | PHE | R | 133 | 9.915 | 36.719 | 105.364 | 1.00 | 34.86 | C |
| ATOM | 3613 | CD1 | PHE | R | 133 | 8.625 | 36.691 | 105.872 | 1.00 | 34.36 | C |
| ATOM | 3615 | CE1 | PHE | R | 133 | 7.738 | 35.680 | 105.506 | 1.00 | 36.41 | C |
| ATOM | 3617 | CZ | PHE | R | 133 | 8.144 | 34.688 | 104.625 | 1.00 | 33.54 | C |
| ATOM | 3619 | CE2 | PHE | R | 133 | 9.433 | 34.704 | 104.117 | 1.00 | 32.03 | C |
| ATOM | 3621 | CD2 | PHE | R | 133 | 10.309 | 35.713 | 104.482 | 1.00 | 33.23 | C |
| ATOM | 3623 | C | PHE | R | 133 | 10.375 | 38.857 | 103.541 | 1.00 | 38.63 | C |
| ATOM | 3624 | O | PHE | R | 133 | 9.219 | 38.806 | 103.128 | 1.00 | 38.95 | O |
| ATOM | 3626 | N | SER | R | 134 | 11.442 | 38.686 | 102.765 | 1.00 | 39.15 | N |
| ATOM | 3627 | CA | SER | R | 134 | 11.364 | 38.102 | 101.433 | 1.00 | 38.45 | C |
| ATOM | 3629 | CB | SER | R | 134 | 11.173 | 39.160 | 100.353 | 1.00 | 37.72 | C |
| ATOM | 3632 | OG | SER | R | 134 | 11.217 | 38.544 | 99.069 | 1.00 | 36.41 | O |
| ATOM | 3634 | C | SER | R | 134 | 12.654 | 37.345 | 101.142 | 1.00 | 39.67 | C |
| ATOM | 3635 | O | SER | R | 134 | 13.742 | 37.894 | 101.330 | 1.00 | 38.74 | O |
| ATOM | 3637 | N | PRO | R | 135 | 12.539 | 36.091 | 100.666 | 1.00 | 38.81 | N |
| ATOM | 3638 | CA | PRO | R | 135 | 13.725 | 35.318 | 100.289 | 1.00 | 41.45 | C |
| ATOM | 3640 | CB | PRO | R | 135 | 13.214 | 33.872 | 100.269 | 1.00 | 39.21 | C |
| ATOM | 3643 | CG | PRO | R | 135 | 11.758 | 33.967 | 100.070 | 1.00 | 38.65 | C |
| ATOM | 3646 | CD | PRO | R | 135 | 11.293 | 35.326 | 100.478 | 1.00 | 38.21 | C |
| ATOM | 3649 | C | PRO | R | 135 | 14.342 | 35.701 | 98.929 | 1.00 | 41.66 | C |
| ATOM | 3650 | O | PRO | R | 135 | 15.348 | 35.116 | 98.522 | 1.00 | 41.87 | O |
| ATOM | 3651 | N | GLY | R | 136 | 13.747 | 36.669 | 98.243 | 1.00 | 41.50 | N |
| ATOM | 3652 | CA | GLY | R | 136 | 14.290 | 37.167 | 96.993 | 1.00 | 39.28 | C |
| ATOM | 3655 | C | GLY | R | 136 | 13.666 | 36.483 | 95.802 | 1.00 | 38.37 | C |
| ATOM | 3656 | O | GLY | R | 136 | 12.591 | 35.890 | 95.908 | 1.00 | 37.47 | O |
| ATOM | 3658 | N | ASP | R | 137 | 14.364 | 36.577 | 94.671 | 1.00 | 36.94 | N |
| ATOM | 3659 | CA | ASP | R | 137 | 13.882 | 36.129 | 93.372 | 1.00 | 37.31 | C |
| ATOM | 3661 | CB | ASP | R | 137 | 13.929 | 34.606 | 93.257 | 1.00 | 37.83 | C |
| ATOM | 3664 | CG | ASP | R | 137 | 14.088 | 34.134 | 91.825 | 1.00 | 36.77 | C |
| ATOM | 3665 | OD1 | ASP | R | 137 | 14.909 | 34.715 | 91.078 | 1.00 | 39.27 | O |
| ATOM | 3666 | OD2 | ASP | R | 137 | 13.402 | 33.172 | 91.436 | 1.00 | 36.00 | O |
| ATOM | 3667 | C | ASP | R | 137 | 12.490 | 36.668 | 93.063 | 1.00 | 36.94 | C |
| ATOM | 3668 | O | ASP | R | 137 | 11.603 | 35.930 | 92.633 | 1.00 | 37.76 | O |
| ATOM | 3670 | N | ASN | R | 138 | 12.330 | 37.971 | 93.287 | 1.00 | 36.22 | N |
| ATOM | 3671 | CA | ASN | R | 138 | 11.114 | 38.731 | 92.956 | 1.00 | 38.59 | C |
| ATOM | 3673 | CB | ASN | R | 138 | 10.785 | 38.627 | 91.455 | 1.00 | 37.92 | C |
| ATOM | 3676 | CG | ASN | R | 138 | 11.579 | 39.607 | 90.620 | 1.00 | 36.63 | C |
| ATOM | 3677 | OD1 | ASN | R | 138 | 12.536 | 40.229 | 91.097 | 1.00 | 34.49 | O |
| ATOM | 3678 | ND2 | ASN | R | 138 | 11.185 | 39.754 | 89.364 | 1.00 | 35.73 | N |
| ATOM | 3681 | C | ASN | R | 138 | 9.877 | 38.446 | 93.806 | 1.00 | 39.02 | C |
| ATOM | 3682 | O | ASN | R | 138 | 8.805 | 38.999 | 93.545 | 1.00 | 39.34 | O |
| ATOM | 3684 | N | GLN | R | 139 | 10.029 | 37.629 | 94.845 | 1.00 | 41.14 | N |
| ATOM | 3685 | CA | GLN | R | 139 | 8.921 | 37.344 | 95.754 | 1.00 | 39.94 | C |
| ATOM | 3687 | CB | GLN | R | 139 | 9.258 | 36.158 | 96.667 | 1.00 | 39.35 | C |
| ATOM | 3690 | CG | GLN | R | 139 | 9.219 | 34.817 | 95.956 | 1.00 | 40.12 | C |
| ATOM | 3693 | CD | GLN | R | 139 | 9.736 | 33.681 | 96.817 | 1.00 | 42.94 | C |
| ATOM | 3694 | OE1 | GLN | R | 139 | 8.956 | 32.922 | 97.405 | 1.00 | 44.07 | O |
| ATOM | 3695 | NE2 | GLN | R | 139 | 11.058 | 33.558 | 96.898 | 1.00 | 42.85 | N |
| ATOM | 3698 | C | GLN | R | 139 | 8.605 | 38.597 | 96.571 | 1.00 | 38.77 | C |
| ATOM | 3699 | O | GLN | R | 139 | 9.513 | 39.320 | 96.977 | 1.00 | 40.35 | O |
| ATOM | 3701 | N | ALA | R | 140 | 7.317 | 38.859 | 96.785 | 1.00 | 41.14 | N |
| ATOM | 3702 | CA | ALA | R | 140 | 6.877 | 40.005 | 97.582 | 1.00 | 40.41 | C |
| ATOM | 3704 | CB | ALA | R | 140 | 5.379 | 40.214 | 97.412 | 1.00 | 41.01 | C |
| ATOM | 3708 | C | ALA | R | 140 | 7.207 | 39.787 | 99.055 | 1.00 | 40.86 | C |
| ATOM | 3709 | O | ALA | R | 140 | 7.257 | 38.651 | 99.517 | 1.00 | 43.34 | O |
| ATOM | 3711 | N | CYS | R | 141 | 7.432 | 40.877 | 99.785 | 1.00 | 40.40 | N |
| ATOM | 3712 | CA | CYS | R | 141 | 7.644 | 40.810 | 101.227 | 1.00 | 40.41 | C |
| ATOM | 3714 | CB | CYS | R | 141 | 8.141 | 42.157 | 101.766 | 1.00 | 41.14 | C |
| ATOM | 3717 | SG | CYS | R | 141 | 9.691 | 42.763 | 101.034 | 1.00 | 39.82 | S |
| ATOM | 3719 | C | CYS | R | 141 | 6.345 | 40.405 | 101.935 | 1.00 | 41.01 | C |
| ATOM | 3720 | O | CYS | R | 141 | 5.258 | 40.740 | 101.466 | 1.00 | 41.62 | O |
| ATOM | 3722 | N | LYS | R | 142 | 6.461 | 39.679 | 103.048 | 1.00 | 40.34 | N |
| ATOM | 3723 | CA | LYS | R | 142 | 5.297 | 39.248 | 103.834 | 1.00 | 41.12 | C |
| ATOM | 3725 | CB | LYS | R | 142 | 4.986 | 37.766 | 103.587 | 1.00 | 43.48 | C |
| ATOM | 3728 | CG | LYS | R | 142 | 4.707 | 37.397 | 102.133 | 1.00 | 44.52 | C |
| ATOM | 3731 | CD | LYS | R | 142 | 3.355 | 37.905 | 101.638 | 1.00 | 45.78 | C |
| ATOM | 3734 | CE | LYS | R | 142 | 3.233 | 37.756 | 100.120 | 1.00 | 45.68 | C |
| ATOM | 3737 | NZ | LYS | R | 142 | 1.826 | 37.613 | 99.656 | 1.00 | 44.97 | N |
| ATOM | 3741 | C | LYS | R | 142 | 5.550 | 39.472 | 105.329 | 1.00 | 41.97 | C |
| ATOM | 3742 | O | LYS | R | 142 | 6.702 | 39.453 | 105.766 | 1.00 | 43.45 | O |
| ATOM | 3744 | N | PRO | R | 143 | 4.478 | 39.683 | 106.120 | 1.00 | 40.81 | N |
| ATOM | 3745 | CA | PRO | R | 143 | 4.662 | 39.890 | 107.564 | 1.00 | 42.56 | C |
| ATOM | 3747 | CB | PRO | R | 143 | 3.236 | 40.159 | 108.083 | 1.00 | 41.51 | C |
| ATOM | 3750 | CG | PRO | R | 143 | 2.411 | 40.449 | 106.892 | 1.00 | 42.05 | C |
| ATOM | 3753 | CD | PRO | R | 143 | 3.059 | 39.753 | 105.731 | 1.00 | 42.03 | C |
| ATOM | 3756 | C | PRO | R | 143 | 5.250 | 38.670 | 108.271 | 1.00 | 44.65 | C |
| ATOM | 3757 | O | PRO | R | 143 | 4.960 | 37.533 | 107.886 | 1.00 | 46.62 | O |
| ATOM | 3758 | N | TRP | R | 144 | 6.069 | 38.905 | 109.295 | 1.00 | 44.51 | N |
| ATOM | 3759 | CA | TRP | R | 144 | 6.599 | 37.812 | 110.093 | 1.00 | 42.77 | C |
| ATOM | 3761 | CB | TRP | R | 144 | 7.599 | 38.303 | 111.145 | 1.00 | 42.23 | C |
| ATOM | 3764 | CG | TRP | R | 144 | 8.873 | 38.894 | 110.618 | 1.00 | 42.65 | C |
| ATOM | 3765 | CD1 | TRP | R | 144 | 9.416 | 38.723 | 109.373 | 1.00 | 43.34 | C |
| ATOM | 3767 | NE1 | TRP | R | 144 | 10.603 | 39.413 | 109.278 | 1.00 | 43.26 | N |
| ATOM | 3769 | CE2 | TRP | R | 144 | 10.859 | 40.028 | 110.476 | 1.00 | 40.70 | C |
| ATOM | 3770 | CD2 | TRP | R | 144 | 9.794 | 39.717 | 111.347 | 1.00 | 41.04 | C |
| ATOM | 3771 | CE3 | TRP | R | 144 | 9.814 | 40.232 | 112.651 | 1.00 | 41.45 | C |
| ATOM | 3773 | CZ3 | TRP | R | 144 | 10.885 | 41.035 | 113.038 | 1.00 | 41.91 | C |
| ATOM | 3775 | CH2 | TRP | R | 144 | 11.929 | 41.325 | 112.148 | 1.00 | 42.29 | C |
| ATOM | 3777 | CZ2 | TRP | R | 144 | 11.935 | 40.832 | 110.866 | 1.00 | 41.85 | C |
| ATOM | 3779 | C | TRP | R | 144 | 5.446 | 37.132 | 110.817 | 1.00 | 44.64 | C |
| ATOM | 3780 | O | TRP | R | 144 | 4.491 | 37.793 | 111.247 | 1.00 | 42.53 | O |
| ATOM | 3782 | N | THR | R | 145 | 5.534 | 35.812 | 110.950 | 1.00 | 44.76 | N |
| ATOM | 3783 | CA | THR | R | 145 | 4.658 | 35.084 | 111.856 | 1.00 | 43.71 | C |
| ATOM | 3785 | CB | THR | R | 145 | 4.771 | 33.560 | 111.660 | 1.00 | 44.06 | C |
| ATOM | 3787 | OG1 | THR | R | 145 | 4.455 | 33.229 | 110.299 | 1.00 | 47.36 | O |
| ATOM | 3789 | CG2 | THR | R | 145 | 3.818 | 32.817 | 112.592 | 1.00 | 43.31 | C |
| ATOM | 3793 | C | THR | R | 145 | 5.052 | 35.453 | 113.286 | 1.00 | 43.22 | C |
| ATOM | 3794 | O | THR | R | 145 | 6.211 | 35.290 | 113.680 | 1.00 | 41.08 | O |
| ATOM | 3796 | N | ASN | R | 146 | 4.091 | 35.983 | 114.039 | 1.00 | 42.64 | N |
| ATOM | 3797 | CA | ASN | R | 146 | 4.267 | 36.224 | 115.463 | 1.00 | 43.24 | C |
| ATOM | 3799 | CB | ASN | R | 146 | 3.344 | 37.359 | 115.937 | 1.00 | 44.73 | C |
| ATOM | 3802 | CG | ASN | R | 146 | 3.729 | 37.905 | 117.307 | 1.00 | 44.33 | C |
| ATOM | 3803 | OD1 | ASN | R | 146 | 4.306 | 37.203 | 118.140 | 1.00 | 44.55 | O |
| ATOM | 3804 | ND2 | ASN | R | 146 | 3.401 | 39.170 | 117.544 | 1.00 | 45.73 | N |
| ATOM | 3807 | C | ASN | R | 146 | 3.961 | 34.922 | 116.206 | 1.00 | 42.81 | C |
| ATOM | 3808 | O | ASN | R | 146 | 2.796 | 34.558 | 116.385 | 1.00 | 40.36 | O |
| ATOM | 3810 | N | CYS | R | 147 | 5.014 | 34.221 | 116.621 | 1.00 | 42.12 | N |
| ATOM | 3811 | CA | CYS | R | 147 | 4.869 | 32.937 | 117.307 | 1.00 | 40.42 | C |
| ATOM | 3813 | CB | CYS | R | 147 | 6.236 | 32.282 | 117.509 | 1.00 | 38.67 | C |
| ATOM | 3816 | SG | CYS | R | 147 | 7.079 | 31.813 | 115.981 | 1.00 | 34.09 | S |
| ATOM | 3818 | C | CYS | R | 147 | 4.161 | 33.068 | 118.652 | 1.00 | 41.13 | C |
| ATOM | 3819 | O | CYS | R | 147 | 3.353 | 32.207 | 119.005 | 1.00 | 41.35 | O |
| ATOM | 3821 | N | THR | R | 148 | 4.467 | 34.138 | 119.391 | 1.00 | 40.48 | N |
| ATOM | 3822 | CA | THR | R | 148 | 3.833 | 34.416 | 120.689 | 1.00 | 40.71 | C |
| ATOM | 3824 | CB | THR | R | 148 | 4.416 | 35.695 | 121.350 | 1.00 | 41.10 | C |
| ATOM | 3826 | OG1 | THR | R | 148 | 5.816 | 35.518 | 121.596 | 1.00 | 42.62 | O |
| ATOM | 3828 | CG2 | THR | R | 148 | 3.719 | 36.005 | 122.670 | 1.00 | 40.79 | C |
| ATOM | 3832 | C | THR | R | 148 | 2.316 | 34.579 | 120.576 | 1.00 | 39.73 | C |
| ATOM | 3833 | O | THR | R | 148 | 1.569 | 34.029 | 121.387 | 1.00 | 38.53 | O |
| ATOM | 3835 | N | LEU | R | 149 | 1.877 | 35.334 | 119.572 | 1.00 | 40.44 | N |
| ATOM | 3836 | CA | LEU | R | 149 | 0.453 | 35.614 | 119.349 | 1.00 | 41.77 | C |
| ATOM | 3838 | CB | LEU | R | 149 | 0.271 | 36.504 | 118.108 | 1.00 | 41.66 | C |
| ATOM | 3841 | CG | LEU | R | 149 | −1.106 | 37.140 | 117.886 | 1.00 | 41.83 | C |
| ATOM | 3843 | CD1 | LEU | R | 149 | −1.391 | 38.205 | 118.937 | 1.00 | 41.84 | C |
| ATOM | 3847 | CD2 | LEU | R | 149 | −1.200 | 37.735 | 116.488 | 1.00 | 42.65 | C |
| ATOM | 3851 | C | LEU | R | 149 | −0.374 | 34.335 | 119.196 | 1.00 | 41.84 | C |
| ATOM | 3852 | O | LEU | R | 149 | −1.528 | 34.285 | 119.628 | 1.00 | 41.45 | O |
| ATOM | 3854 | N | ALA | R | 150 | 0.223 | 33.308 | 118.591 | 1.00 | 42.38 | N |
| ATOM | 3855 | CA | ALA | R | 150 | −0.442 | 32.019 | 118.390 | 1.00 | 43.11 | C |
| ATOM | 3857 | CB | ALA | R | 150 | −0.041 | 31.431 | 117.042 | 1.00 | 44.51 | C |
| ATOM | 3861 | C | ALA | R | 150 | −0.159 | 31.013 | 119.513 | 1.00 | 44.05 | C |
| ATOM | 3862 | O | ALA | R | 150 | −0.392 | 29.817 | 119.340 | 1.00 | 46.97 | O |
| ATOM | 3864 | N | GLY | R | 151 | 0.330 | 31.491 | 120.657 | 1.00 | 43.36 | N |
| ATOM | 3865 | CA | GLY | R | 151 | 0.579 | 30.632 | 121.818 | 1.00 | 42.94 | C |
| ATOM | 3868 | C | GLY | R | 151 | 1.696 | 29.626 | 121.610 | 1.00 | 43.06 | C |
| ATOM | 3869 | O | GLY | R | 151 | 1.607 | 28.490 | 122.076 | 1.00 | 42.12 | O |
| ATOM | 3871 | N | LYS | R | 152 | 2.749 | 30.051 | 120.912 | 1.00 | 43.51 | N |
| ATOM | 3872 | CA | LYS | R | 152 | 3.898 | 29.196 | 120.600 | 1.00 | 43.33 | C |
| ATOM | 3874 | CB | LYS | R | 152 | 3.900 | 28.834 | 119.109 | 1.00 | 43.47 | C |
| ATOM | 3877 | CG | LYS | R | 152 | 2.754 | 27.931 | 118.679 | 1.00 | 43.78 | C |
| ATOM | 3880 | CD | LYS | R | 152 | 2.758 | 27.715 | 117.175 | 1.00 | 43.72 | C |
| ATOM | 3883 | CE | LYS | R | 152 | 1.692 | 26.720 | 116.739 | 1.00 | 44.14 | C |
| ATOM | 3886 | NZ | LYS | R | 152 | 1.904 | 26.267 | 115.331 | 1.00 | 44.91 | N |
| ATOM | 3890 | C | LYS | R | 152 | 5.209 | 29.898 | 120.952 | 1.00 | 43.03 | C |
| ATOM | 3891 | O | LYS | R | 152 | 5.271 | 31.128 | 121.010 | 1.00 | 42.11 | O |
| ATOM | 3893 | N | HIS | R | 153 | 6.253 | 29.109 | 121.191 | 1.00 | 43.26 | N |
| ATOM | 3894 | CA | HIS | R | 153 | 7.599 | 29.653 | 121.369 | 1.00 | 43.07 | C |
| ATOM | 3896 | CB | HIS | R | 153 | 8.438 | 28.769 | 122.301 | 1.00 | 43.57 | C |
| ATOM | 3899 | CG | HIS | R | 153 | 8.086 | 28.917 | 123.750 | 1.00 | 44.38 | C |
| ATOM | 3900 | ND1 | HIS | R | 153 | 8.256 | 30.099 | 124.439 | 1.00 | 44.13 | N |
| ATOM | 3902 | CE1 | HIS | R | 153 | 7.869 | 29.935 | 125.692 | 1.00 | 45.41 | C |
| ATOM | 3904 | NE2 | HIS | R | 153 | 7.455 | 28.689 | 125.842 | 1.00 | 44.67 | N |
| ATOM | 3906 | CD2 | HIS | R | 153 | 7.581 | 28.031 | 124.643 | 1.00 | 45.16 | C |
| ATOM | 3908 | C | HIS | R | 153 | 8.270 | 29.792 | 120.008 | 1.00 | 41.54 | C |
| ATOM | 3909 | O | HIS | R | 153 | 7.887 | 29.121 | 119.050 | 1.00 | 40.10 | O |
| ATOM | 3911 | N | THR | R | 154 | 9.267 | 30.669 | 119.932 | 1.00 | 41.95 | N |
| ATOM | 3912 | CA | THR | R | 154 | 10.006 | 30.898 | 118.695 | 1.00 | 42.63 | C |
| ATOM | 3914 | CB | THR | R | 154 | 10.419 | 32.381 | 118.553 | 1.00 | 42.68 | C |
| ATOM | 3916 | OG1 | THR | R | 154 | 9.249 | 33.209 | 118.572 | 1.00 | 42.70 | O |
| ATOM | 3918 | CG2 | THR | R | 154 | 11.178 | 32.620 | 117.245 | 1.00 | 42.48 | C |
| ATOM | 3922 | C | THR | R | 154 | 11.254 | 30.016 | 118.629 | 1.00 | 42.39 | C |
| ATOM | 3923 | O | THR | R | 154 | 12.181 | 30.176 | 119.423 | 1.00 | 38.85 | O |
| ATOM | 3925 | N | LEU | R | 155 | 11.268 | 29.096 | 117.667 | 1.00 | 44.83 | N |
| ATOM | 3926 | CA | LEU | R | 155 | 12.427 | 28.240 | 117.408 | 1.00 | 46.06 | C |
| ATOM | 3928 | CB | LEU | R | 155 | 12.023 | 27.046 | 116.527 | 1.00 | 47.02 | C |
| ATOM | 3931 | CG | LEU | R | 155 | 12.654 | 25.694 | 116.855 | 1.00 | 47.55 | C |
| ATOM | 3933 | CD1 | LEU | R | 155 | 12.021 | 25.138 | 118.108 | 1.00 | 47.32 | C |
| ATOM | 3937 | CD2 | LEU | R | 155 | 12.494 | 24.717 | 115.690 | 1.00 | 47.77 | C |
| ATOM | 3941 | C | LEU | R | 155 | 13.528 | 29.054 | 116.716 | 1.00 | 46.82 | C |
| ATOM | 3942 | O | LEU | R | 155 | 14.638 | 29.180 | 117.240 | 1.00 | 45.93 | O |
| ATOM | 3944 | N | GLN | R | 156 | 13.192 | 29.609 | 115.546 | 1.00 | 46.72 | N |
| ATOM | 3945 | CA | GLN | R | 156 | 14.112 | 30.408 | 114.729 | 1.00 | 46.66 | C |
| ATOM | 3947 | CB | GLN | R | 156 | 14.175 | 29.864 | 113.294 | 1.00 | 47.19 | C |
| ATOM | 3950 | CG | GLN | R | 156 | 14.437 | 28.359 | 113.168 | 1.00 | 47.78 | C |
| ATOM | 3953 | CD | GLN | R | 156 | 15.911 | 28.004 | 113.106 | 1.00 | 47.55 | C |
| ATOM | 3954 | OE1 | GLN | R | 156 | 16.652 | 28.518 | 112.265 | 1.00 | 47.82 | O |
| ATOM | 3955 | NE2 | GLN | R | 156 | 16.339 | 27.103 | 113.984 | 1.00 | 46.18 | N |
| ATOM | 3958 | C | GLN | R | 156 | 13.614 | 31.853 | 114.677 | 1.00 | 46.30 | C |
| ATOM | 3959 | O | GLN | R | 156 | 12.457 | 32.087 | 114.334 | 1.00 | 45.60 | O |
| ATOM | 3961 | N | PRO | R | 157 | 14.467 | 32.828 | 115.044 | 1.00 | 47.61 | N |
| ATOM | 3962 | CA | PRO | R | 157 | 14.087 | 34.227 | 114.837 | 1.00 | 46.46 | C |
| ATOM | 3964 | CB | PRO | R | 157 | 15.285 | 35.009 | 115.396 | 1.00 | 47.25 | C |
| ATOM | 3967 | CG | PRO | R | 157 | 15.985 | 34.064 | 116.298 | 1.00 | 48.32 | C |
| ATOM | 3970 | CD | PRO | R | 157 | 15.779 | 32.709 | 115.706 | 1.00 | 48.43 | C |
| ATOM | 3973 | C | PRO | R | 157 | 13.864 | 34.579 | 113.360 | 1.00 | 47.30 | C |
| ATOM | 3974 | O | PRO | R | 157 | 14.564 | 34.067 | 112.476 | 1.00 | 47.60 | O |
| ATOM | 3975 | N | ALA | R | 158 | 12.889 | 35.447 | 113.108 | 1.00 | 45.44 | N |
| ATOM | 3976 | CA | ALA | R | 158 | 12.627 | 35.945 | 111.765 | 1.00 | 43.40 | C |
| ATOM | 3978 | CB | ALA | R | 158 | 11.289 | 36.666 | 111.718 | 1.00 | 40.55 | C |
| ATOM | 3982 | C | ALA | R | 158 | 13.751 | 36.885 | 111.338 | 1.00 | 44.62 | C |
| ATOM | 3983 | O | ALA | R | 158 | 14.354 | 37.564 | 112.179 | 1.00 | 47.19 | O |
| ATOM | 3985 | N | SER | R | 159 | 14.042 | 36.899 | 110.039 | 1.00 | 42.68 | N |
| ATOM | 3986 | CA | SER | R | 159 | 15.047 | 37.805 | 109.467 | 1.00 | 42.78 | C |
| ATOM | 3988 | CB | SER | R | 159 | 16.297 | 37.028 | 109.031 | 1.00 | 40.46 | C |
| ATOM | 3991 | OG | SER | R | 159 | 16.044 | 36.225 | 107.887 | 1.00 | 38.69 | O |
| ATOM | 3993 | C | SER | R | 159 | 14.417 | 38.527 | 108.284 | 1.00 | 43.06 | C |
| ATOM | 3994 | O | SER | R | 159 | 13.224 | 38.370 | 108.035 | 1.00 | 45.34 | O |
| ATOM | 3996 | N | ASN | R | 160 | 15.201 | 39.324 | 107.560 | 1.00 | 44.08 | N |
| ATOM | 3997 | CA | ASN | R | 160 | 14.692 | 39.973 | 106.342 | 1.00 | 43.32 | C |
| ATOM | 3999 | CB | ASN | R | 160 | 15.569 | 41.169 | 105.920 | 1.00 | 44.24 | C |
| ATOM | 4002 | CG | ASN | R | 160 | 16.989 | 40.773 | 105.504 | 1.00 | 45.15 | C |
| ATOM | 4003 | OD1 | ASN | R | 160 | 17.238 | 39.658 | 105.034 | 1.00 | 44.63 | O |
| ATOM | 4004 | ND2 | ASN | R | 160 | 17.923 | 41.718 | 105.657 | 1.00 | 45.43 | N |
| ATOM | 4007 | C | ASN | R | 160 | 14.488 | 38.990 | 105.175 | 1.00 | 41.69 | C |
| ATOM | 4008 | O | ASN | R | 160 | 13.734 | 39.279 | 104.251 | 1.00 | 40.07 | O |
| ATOM | 4010 | N | SER | R | 161 | 15.156 | 37.836 | 105.237 | 1.00 | 41.10 | N |
| ATOM | 4011 | CA | SER | R | 161 | 15.111 | 36.834 | 104.170 | 1.00 | 40.06 | C |
| ATOM | 4013 | CB | SER | R | 161 | 16.536 | 36.395 | 103.792 | 1.00 | 40.18 | C |
| ATOM | 4016 | OG | SER | R | 161 | 17.204 | 35.759 | 104.869 | 1.00 | 39.54 | O |
| ATOM | 4018 | C | SER | R | 161 | 14.258 | 35.610 | 104.507 | 1.00 | 38.95 | C |
| ATOM | 4019 | O | SER | R | 161 | 13.970 | 34.798 | 103.630 | 1.00 | 44.04 | O |
| ATOM | 4021 | N | SER | R | 162 | 13.857 | 35.464 | 105.765 | 1.00 | 39.95 | N |
| ATOM | 4022 | CA | SER | R | 162 | 13.050 | 34.315 | 106.166 | 1.00 | 39.08 | C |
| ATOM | 4024 | CB | SER | R | 162 | 13.942 | 33.106 | 106.465 | 1.00 | 39.18 | C |
| ATOM | 4027 | OG | SER | R | 162 | 14.923 | 33.423 | 107.434 | 1.00 | 40.71 | O |
| ATOM | 4029 | C | SER | R | 162 | 12.155 | 34.608 | 107.361 | 1.00 | 38.75 | C |
| ATOM | 4030 | O | SER | R | 162 | 12.400 | 35.528 | 108.136 | 1.00 | 39.38 | O |
| ATOM | 4032 | N | ASP | R | 163 | 11.108 | 33.802 | 107.489 | 1.00 | 41.71 | N |
| ATOM | 4033 | CA | ASP | R | 163 | 10.136 | 33.934 | 108.566 | 1.00 | 41.19 | C |
| ATOM | 4035 | CB | ASP | R | 163 | 8.796 | 33.331 | 108.125 | 1.00 | 41.36 | C |
| ATOM | 4038 | CG | ASP | R | 163 | 7.620 | 33.843 | 108.929 | 1.00 | 43.13 | C |
| ATOM | 4039 | OD1 | ASP | R | 163 | 7.793 | 34.771 | 109.750 | 1.00 | 44.94 | O |
| ATOM | 4040 | OD2 | ASP | R | 163 | 6.507 | 33.309 | 108.747 | 1.00 | 43.35 | O |
| ATOM | 4041 | C | ASP | R | 163 | 10.646 | 33.207 | 109.801 | 1.00 | 42.61 | C |
| ATOM | 4042 | O | ASP | R | 163 | 11.543 | 32.355 | 109.711 | 1.00 | 42.49 | O |
| ATOM | 4044 | N | ALA | R | 164 | 10.082 | 33.561 | 110.954 | 1.00 | 42.18 | N |
| ATOM | 4045 | CA | ALA | R | 164 | 10.307 | 32.814 | 112.184 | 1.00 | 41.34 | C |
| ATOM | 4047 | CB | ALA | R | 164 | 9.727 | 33.561 | 113.388 | 1.00 | 39.51 | C |
| ATOM | 4051 | C | ALA | R | 164 | 9.680 | 31.426 | 112.078 | 1.00 | 39.87 | C |
| ATOM | 4052 | O | ALA | R | 164 | 8.655 | 31.238 | 111.420 | 1.00 | 38.54 | O |
| ATOM | 4054 | N | ILE | R | 165 | 10.309 | 30.452 | 112.723 | 1.00 | 42.44 | N |
| ATOM | 4055 | CA | ILE | R | 165 | 9.721 | 29.122 | 112.851 | 1.00 | 42.23 | C |
| ATOM | 4057 | CB | ILE | R | 165 | 10.682 | 28.013 | 112.357 | 1.00 | 42.14 | C |
| ATOM | 4059 | CG1 | ILE | R | 165 | 11.005 | 28.246 | 110.870 | 1.00 | 42.01 | C |
| ATOM | 4062 | CD1 | ILE | R | 165 | 11.934 | 27.228 | 110.243 | 1.00 | 40.75 | C |
| ATOM | 4066 | CG2 | ILE | R | 165 | 10.057 | 26.631 | 112.573 | 1.00 | 42.08 | C |
| ATOM | 4070 | C | ILE | R | 165 | 9.320 | 28.935 | 114.313 | 1.00 | 41.00 | C |
| ATOM | 4071 | O | ILE | R | 165 | 10.107 | 29.222 | 115.216 | 1.00 | 38.53 | O |
| ATOM | 4073 | N | CYS | R | 166 | 8.078 | 28.498 | 114.529 | 1.00 | 41.63 | N |
| ATOM | 4074 | CA | CYS | R | 166 | 7.542 | 28.271 | 115.874 | 1.00 | 40.25 | C |
| ATOM | 4076 | CB | CYS | R | 166 | 6.136 | 28.874 | 116.010 | 1.00 | 36.60 | C |
| ATOM | 4079 | SG | CYS | R | 166 | 5.823 | 30.442 | 115.133 | 1.00 | 35.73 | S |
| ATOM | 4081 | C | CYS | R | 166 | 7.472 | 26.773 | 116.179 | 1.00 | 40.69 | C |
| ATOM | 4082 | O | CYS | R | 166 | 7.720 | 25.938 | 115.310 | 1.00 | 40.17 | O |
| ATOM | 4084 | N | GLU | R | 167 | 7.135 | 26.451 | 117.426 | 1.00 | 44.50 | N |
| ATOM | 4085 | CA | GLU | R | 167 | 6.860 | 25.077 | 117.849 | 1.00 | 44.93 | C |
| ATOM | 4087 | CB | GLU | R | 167 | 8.151 | 24.372 | 118.282 | 1.00 | 45.04 | C |
| ATOM | 4090 | CG | GLU | R | 167 | 7.972 | 22.906 | 118.691 | 1.00 | 44.95 | C |
| ATOM | 4093 | CD | GLU | R | 167 | 9.225 | 22.293 | 119.289 | 1.00 | 45.34 | C |
| ATOM | 4094 | OE1 | GLU | R | 167 | 10.329 | 22.577 | 118.776 | 1.00 | 40.92 | O |
| ATOM | 4095 | OE2 | GLU | R | 167 | 9.102 | 21.517 | 120.273 | 1.00 | 47.99 | O |
| ATOM | 4096 | C | GLU | R | 167 | 5.865 | 25.121 | 119.004 | 1.00 | 47.65 | C |
| ATOM | 4097 | O | GLU | R | 167 | 5.782 | 26.127 | 119.711 | 1.00 | 49.69 | O |
| ATOM | 4099 | N | ASP | R | 168 | 5.114 | 24.035 | 119.192 | 1.00 | 49.16 | N |
| ATOM | 4100 | CA | ASP | R | 168 | 4.136 | 23.945 | 120.280 | 1.00 | 48.85 | C |
| ATOM | 4102 | CB | ASP | R | 168 | 3.139 | 22.811 | 120.018 | 1.00 | 50.72 | C |
| ATOM | 4105 | CG | ASP | R | 168 | 2.174 | 23.131 | 118.886 | 1.00 | 52.34 | C |
| ATOM | 4106 | OD1 | ASP | R | 168 | 2.637 | 23.492 | 117.784 | 1.00 | 53.71 | O |
| ATOM | 4107 | OD2 | ASP | R | 168 | 0.949 | 23.020 | 119.096 | 1.00 | 54.20 | O |
| ATOM | 4108 | C | ASP | R | 168 | 4.808 | 23.753 | 121.639 | 1.00 | 49.72 | C |
| ATOM | 4109 | O | ASP | R | 168 | 5.737 | 22.957 | 121.781 | 1.00 | 50.73 | O |
| ATOM | 4111 | C1 | NAG | R | 200 | 19.280 | 41.261 | 105.448 | 1.00 | 49.92 | C |
| ATOM | 4114 | C2 | NAG | R | 200 | 19.879 | 42.650 | 105.162 | 1.00 | 50.89 | C |
| ATOM | 4116 | N2 | NAG | R | 200 | 19.329 | 43.233 | 103.933 | 1.00 | 48.22 | N |
| ATOM | 4118 | C7 | NAG | R | 200 | 19.970 | 43.309 | 102.766 | 1.00 | 49.18 | C |
| ATOM | 4119 | O7 | NAG | R | 200 | 21.108 | 43.764 | 102.636 | 1.00 | 51.17 | O |
| ATOM | 4120 | C8 | NAG | R | 200 | 19.219 | 42.833 | 101.558 | 1.00 | 48.74 | C |
| ATOM | 4124 | C3 | NAG | R | 200 | 21.414 | 42.622 | 105.228 | 1.00 | 52.36 | C |
| ATOM | 4126 | O3 | NAG | R | 200 | 21.973 | 43.919 | 105.134 | 1.00 | 52.44 | O |
| ATOM | 4128 | C4 | NAG | R | 200 | 21.881 | 41.968 | 106.522 | 1.00 | 53.18 | C |
| ATOM | 4130 | O4 | NAG | R | 200 | 23.290 | 41.862 | 106.507 | 1.00 | 55.04 | O |
| ATOM | 4132 | C5 | NAG | R | 200 | 21.228 | 40.595 | 106.668 | 1.00 | 52.74 | C |
| ATOM | 4134 | C6 | NAG | R | 200 | 21.645 | 39.856 | 107.944 | 1.00 | 52.41 | C |
| ATOM | 4137 | O6 | NAG | R | 200 | 22.679 | 40.524 | 108.636 | 1.00 | 51.52 | O |
| ATOM | 4139 | O5 | NAG | R | 200 | 19.817 | 40.741 | 106.654 | 1.00 | 52.42 | O |
| ATOM | 4140 | O | HOH | W | 1 | 0.003 | 64.605 | 62.043 | 0.33 | 24.72 | O |
| ATOM | 4143 | O | HOH | W | 2 | −9.605 | 59.559 | 73.200 | 1.00 | 26.68 | O |
| ATOM | 4146 | O | HOH | W | 3 | −8.140 | 58.184 | 89.102 | 1.00 | 31.94 | O |
| ATOM | 4149 | O | HOH | W | 4 | −7.063 | 63.539 | 74.010 | 1.00 | 25.66 | O |
| ATOM | 4152 | O | HOH | W | 5 | −4.723 | 59.253 | 82.512 | 1.00 | 37.75 | O |
| ATOM | 4155 | O | HOH | W | 6 | −3.376 | 51.621 | 59.948 | 1.00 | 30.82 | O |
| ATOM | 4158 | O | HOH | W | 7 | −21.301 | 47.919 | 89.797 | 1.00 | 45.00 | O |
| ATOM | 4161 | O | HOH | W | 8 | 17.454 | 35.093 | 91.847 | 1.00 | 36.98 | O |
| ATOM | 4164 | O | HOH | W | 9 | −11.544 | 54.737 | 60.091 | 1.00 | 35.28 | O |
| ATOM | 4167 | O | HOH | W | 10 | 11.517 | 34.054 | 89.321 | 1.00 | 38.62 | O |
| ATOM | 4170 | O | HOH | W | 11 | 15.538 | 37.329 | 60.314 | 1.00 | 40.16 | O |
| ATOM | 4173 | O | HOH | W | 12 | −2.071 | 57.563 | 57.100 | 1.00 | 30.60 | O |
| ATOM | 4176 | O | HOH | W | 13 | −14.156 | 51.778 | 61.686 | 1.00 | 43.45 | O |
| ATOM | 4179 | O | HOH | W | 14 | 9.646 | 34.836 | 59.126 | 1.00 | 30.31 | O |
| ATOM | 4182 | O | HOH | W | 15 | 21.142 | 46.286 | 66.627 | 1.00 | 46.45 | O |
| ATOM | 4185 | O | HOH | W | 16 | −10.386 | 45.662 | 73.129 | 1.00 | 33.78 | O |
| ATOM | 4188 | O | HOH | W | 17 | −16.511 | 59.613 | 72.079 | 1.00 | 31.04 | O |
| ATOM | 4191 | O | HOH | W | 18 | 23.071 | 41.631 | 72.861 | 1.00 | 44.59 | O |
| ATOM | 4194 | O | HOH | W | 19 | 16.837 | 33.637 | 100.166 | 1.00 | 41.27 | O |
| ATOM | 4197 | O | HOH | W | 20 | −14.217 | 62.821 | 74.196 | 1.00 | 32.88 | O |
| ATOM | 4200 | O | HOH | W | 21 | −14.670 | 40.569 | 64.129 | 1.00 | 41.83 | O |
| ATOM | 4203 | O | HOH | W | 22 | −18.656 | 59.058 | 85.064 | 1.00 | 49.03 | O |
| ATOM | 4206 | O | HOH | W | 23 | 15.806 | 33.463 | 88.496 | 1.00 | 37.42 | O |
| ATOM | 4209 | O | HOH | W | 25 | 12.189 | 39.742 | 64.884 | 1.00 | 45.26 | O |
| ATOM | 4212 | O | HOH | W | 26 | −8.792 | 47.345 | 90.359 | 1.00 | 46.94 | O |
| ATOM | 4215 | O | HOH | W | 27 | −2.885 | 42.079 | 69.492 | 1.00 | 30.23 | O |
| ATOM | 4218 | O | HOH | W | 28 | 6.726 | 51.739 | 63.231 | 1.00 | 36.36 | O |
| ATOM | 4221 | O | HOH | W | 29 | −2.489 | 44.580 | 61.826 | 1.00 | 31.20 | O |
| ATOM | 4224 | O | HOH | W | 30 | 3.671 | 43.621 | 63.911 | 1.00 | 29.09 | O |
| ATOM | 4227 | O | HOH | W | 31 | 6.639 | 55.364 | 72.156 | 1.00 | 26.88 | O |
| ATOM | 4230 | O | HOH | W | 32 | −20.935 | 57.981 | 68.149 | 1.00 | 56.53 | O |
| ATOM | 4233 | O | HOH | W | 33 | −5.708 | 40.662 | 69.409 | 1.00 | 40.78 | O |
| ATOM | 4236 | O | HOH | W | 34 | −18.463 | 55.317 | 77.193 | 1.00 | 33.89 | O |
| ATOM | 4239 | O | HOH | W | 35 | 24.111 | 41.732 | 89.693 | 1.00 | 41.89 | O |
| ATOM | 4242 | O | HOH | W | 37 | −7.192 | 51.146 | 54.815 | 1.00 | 42.35 | O |
| ATOM | 4245 | O | HOH | W | 38 | 7.353 | 30.448 | 54.395 | 1.00 | 36.83 | O |
| ATOM | 4248 | O | HOH | W | 39 | −1.856 | 47.579 | 49.816 | 1.00 | 49.91 | O |
| ATOM | 4251 | O | HOH | W | 40 | 1.022 | 45.075 | 74.032 | 1.00 | 31.65 | O |
| ATOM | 4254 | O | HOH | W | 41 | −13.675 | 56.719 | 63.083 | 1.00 | 30.98 | O |
| ATOM | 4257 | O | HOH | W | 42 | 4.184 | 56.101 | 74.625 | 1.00 | 37.55 | O |
| ATOM | 4260 | O | HOH | W | 43 | 6.021 | 49.653 | 70.667 | 1.00 | 45.68 | O |
| ATOM | 4263 | O | HOH | W | 44 | 10.849 | 37.414 | 49.625 | 1.00 | 46.96 | O |
| ATOM | 4266 | O | HOH | W | 45 | 7.908 | 39.387 | 73.942 | 1.00 | 47.08 | O |
| ATOM | 4269 | O | HOH | W | 46 | 17.232 | 35.188 | 60.295 | 1.00 | 37.63 | O |
| ATOM | 4272 | O | HOH | W | 47 | 7.163 | 43.073 | 82.513 | 1.00 | 45.08 | O |
| ATOM | 4275 | O | HOH | W | 48 | 7.348 | 53.460 | 70.376 | 1.00 | 39.99 | O |
| ATOM | 4278 | O | HOH | W | 49 | −22.392 | 53.200 | 86.801 | 1.00 | 35.43 | O |
| ATOM | 4281 | O | HOH | W | 50 | −15.839 | 55.952 | 64.302 | 1.00 | 36.96 | O |
| ATOM | 4284 | O | HOH | W | 51 | −1.641 | 62.755 | 78.227 | 1.00 | 46.06 | O |
| ATOM | 4287 | O | HOH | W | 52 | 18.525 | 50.453 | 86.037 | 1.00 | 38.49 | O |
| ATOM | 4290 | O | HOH | W | 53 | −20.729 | 51.556 | 80.247 | 1.00 | 36.50 | O |
| ATOM | 4293 | O | HOH | W | 54 | 9.596 | 48.525 | 62.029 | 1.00 | 35.06 | O |
| ATOM | 4296 | O | HOH | W | 55 | 21.406 | 37.333 | 65.920 | 1.00 | 37.87 | O |
| ATOM | 4299 | O | HOH | W | 56 | 5.766 | 51.736 | 71.089 | 1.00 | 49.98 | O |
| ATOM | 4302 | O | HOH | W | 58 | 10.804 | 31.713 | 105.358 | 1.00 | 42.64 | O |
| ATOM | 4305 | O | HOH | W | 59 | −4.355 | 45.593 | 56.651 | 1.00 | 42.50 | O |
| ATOM | 4308 | O | HOH | W | 61 | −27.365 | 49.332 | 86.072 | 1.00 | 36.03 | O |
| ATOM | 4311 | O | HOH | W | 62 | −18.249 | 58.520 | 67.105 | 1.00 | 40.31 | O |
| ATOM | 4314 | O | HOH | W | 65 | 13.450 | 36.678 | 57.611 | 1.00 | 37.99 | O |
| ATOM | 4317 | O | HOH | W | 67 | 22.014 | 45.061 | 84.064 | 1.00 | 34.03 | O |
| ATOM | 4320 | O | HOH | W | 68 | −26.144 | 48.249 | 84.250 | 1.00 | 31.91 | O |
| ATOM | 4323 | O | HOH | W | 70 | 9.027 | 35.753 | 50.589 | 1.00 | 49.17 | O |
| ATOM | 4326 | O | HOH | W | 71 | 13.615 | 37.382 | 54.874 | 1.00 | 43.95 | O |
| ATOM | 4329 | O | HOH | W | 72 | −15.933 | 56.390 | 59.505 | 1.00 | 42.61 | O |
| ATOM | 4332 | O | HOH | W | 73 | −19.841 | 50.436 | 89.715 | 1.00 | 42.47 | O |
| ATOM | 4335 | O | HOH | W | 74 | −19.837 | 48.747 | 73.481 | 1.00 | 41.87 | O |
| ATOM | 4338 | O | HOH | W | 76 | 1.873 | 53.977 | 81.007 | 1.00 | 42.67 | O |
| ATOM | 4341 | O | HOH | W | 77 | −11.971 | 45.719 | 75.394 | 1.00 | 41.21 | O |
| ATOM | 4344 | O | HOH | W | 78 | −1.236 | 62.638 | 60.793 | 1.00 | 39.38 | O |
| ATOM | 4347 | O | HOH | W | 79 | 17.280 | 46.618 | 64.398 | 1.00 | 36.58 | O |
| ATOM | 4350 | O | HOH | W | 80 | 17.929 | 49.358 | 66.877 | 1.00 | 62.41 | O |
| ATOM | 4353 | O | HOH | W | 81 | 22.466 | 51.650 | 91.599 | 1.00 | 71.88 | O |
| ATOM | 4356 | O | HOH | W | 82 | 19.089 | 36.624 | 79.306 | 1.00 | 44.88 | O |
| ATOM | 4359 | O | HOH | W | 83 | 13.496 | 38.415 | 76.625 | 1.00 | 38.69 | O |
| ATOM | 4362 | O | HOH | W | 84 | 13.005 | 35.737 | 76.139 | 1.00 | 45.40 | O |
| ATOM | 4365 | O | HOH | W | 85 | 7.425 | 45.305 | 89.345 | 1.00 | 45.36 | O |
| ATOM | 4368 | O | HOH | W | 86 | 13.958 | 41.378 | 84.076 | 1.00 | 42.15 | O |
| ATOM | 4371 | O | HOH | W | 87 | 12.886 | 31.526 | 103.743 | 1.00 | 47.81 | O |
| ATOM | 4374 | O | HOH | W | 88 | 7.844 | 35.098 | 92.726 | 1.00 | 49.59 | O |
| ATOM | 4377 | O | HOH | W | 89 | −18.830 | 48.613 | 65.744 | 1.00 | 52.05 | O |
| ATOM | 4380 | O | HOH | W | 90 | −14.910 | 48.132 | 61.613 | 1.00 | 47.14 | O |
| ATOM | 4383 | O | HOH | W | 91 | −13.808 | 51.473 | 93.782 | 1.00 | 49.41 | O |
| ATOM | 4386 | O | HOH | W | 92 | −18.040 | 53.288 | 92.965 | 1.00 | 47.90 | O |
| ATOM | 4389 | O | HOH | W | 93 | −14.768 | 58.203 | 87.005 | 1.00 | 34.70 | O |
| ATOM | 4392 | O | HOH | W | 94 | 2.625 | 54.056 | 73.796 | 1.00 | 38.73 | O |
| ATOM | 4395 | O | HOH | W | 95 | −26.673 | 53.342 | 85.864 | 1.00 | 39.34 | O |
| ATOM | 4398 | O | HOH | W | 96 | −22.448 | 52.552 | 89.977 | 1.00 | 57.76 | O |

1-20. (canceled)

21. A method for obtaining structural information about a molecule or molecular complex comprising application of at least a portion of the mOX40L structure coordinates listed in Table 8 or 9 to an X-ray diffraction pattern of the molecule or molecular complex's crystal structure to generate a three-dimensional electron density map of at least a portion of the molecule or molecular complex.

22. (canceled)

23. A computer-assisted method for identifying an agent that modulates mOX40L activity comprising:
(a) providing a computer modeling application with a set of structure coordinates of Table 8 or 9 defining at least a portion of a mOX40L binding site;
(b) providing the computer modeling application with a set of structure coordinates for a test agent; and
(c) modeling the structure of (a) complexed with (b) to determine if the test agent associates with the mOX40L binding site and optionally determining if the test agent modulates binding or activity of mOX40L.

24. (canceled)

25. A method of identifying a molecule that mimics mOX40L comprising:
a) searching a molecular structure database with the structural coordinates of Table 8 or 9; and
b) selecting a molecule from the database that mimics the structural coordinates of the mOX40L and optionally determining if the molecule modulates binding or activity of mOX40L.

26-44. (canceled)

45. A method for obtaining structural information about a molecule or molecular complex comprising application of at least a portion of the hOX40L structure coordinates listed in Table 10 to an X-ray diffraction pattern of the molecule or molecular complex's crystal structure to generate a three-dimensional electron density map of at least a portion of the molecule or molecular complex.

46. (canceled)

47. A computer-assisted method for identifying an agent that modulates hOX40L activity comprising:
(a) providing a computer modeling application with a set of structure coordinates of Table 10 defining at least a portion of a hOX40L binding site;
(b) providing the computer modeling application with a set of structure coordinates for a test agent; and
(c) modeling the structure of (a) complexed with (b) to determine if the test agent associates with the hOX40L binding site and optionally determining if the test agent modulates binding or activity of hOX40L.

48. (canceled)

49. A method of identifying a molecule that mimics hOX40L comprising:
a) searching a molecular structure database with the structural coordinates of Table 10; and
b) selecting a molecule from the database that mimics the structural coordinates of the hOX40L and optionally determining if the molecule modulates binding or activity of hOX40L.

50-67. (canceled)

68. A method for obtaining structural information about a molecule or molecular complex comprising application of at least a portion of the hOX40 receptor structure coordinates listed in Table 9 or 10 to an X-ray diffraction pattern of the molecule or molecular complex's crystal structure to generate a three-dimensional electron density map of at least a portion of the molecule or molecular complex.

69. (canceled)

70. A computer-assisted method for identifying an agent that modulates hOX40 receptor activity comprising:
(a) providing a computer modeling application with a set of structure coordinates of Table 9 or 10 defining at least a portion of a hOX40 receptor binding site for mOX40L or hOX40L;
(b) providing the computer modeling application with a set of structure coordinates for a test agent; and
(c) modeling the structure of (a) complexed with (b) to determine if the test agent associates with the hOX40 receptor binding site and optionally determining if the test agent modulates binding or activity of hOX40 receptor.

71. (canceled)

72. A method of identifying a molecule that mimics the hOX40 receptor comprising:
a) searching a molecular structure database with the structural coordinates of Table 9 or 10; and
b) selecting a molecule from the database that mimics the structural coordinates of the hOX40 receptor and optionally determining if the molecule modulates binding or activity of hOX40 receptor.

73-89. (canceled)

90. A crystal comprising a OX40L,
wherein the OX40L comprises a polypeptide having at least 90% sequence identity to a polypeptide selected from the group consisting of: a polypeptide comprising the amino acid sequence of amino acids 51-183 of SEQ ID NO:3, and a polypeptide comprising the amino acid sequence of amino acids 51-198 of SEQ ID NO:1, and
wherein the polypeptide has at least one of the following characteristics: binds to a hOX40 receptor or a hOX40 receptor ligand binding fragment thereof, and activates a hOX40 receptor or a hOX40 receptor ligand binding fragment thereof.

91. The crystal of claim 90, wherein the OX40L comprises a polypeptide comprising the amino acid sequence of amino acids 51-198 of SEQ ID NO:1, and wherein the crystal has a space group symmetry of P6₃ and a unit cell having the dimensions of a=b and are about 74 Å, and c is about 48 Å.

92. The crystal of claim 90, wherein the OX40L comprises a polypeptide comprising the amino acid sequence of amino acids 51-183 of SEQ ID NO:3, wherein SEQ ID NO: 3 has the following mutations: N90D and N114D.

93. The crystal of claim 90, further comprising a ligand, wherein the ligand is complexed with the OX40L, wherein the OX40L comprises a polypeptide comprising the amino acid sequence of amino acids 51-198 of SEQ ID NO:1, and wherein the ligand comprises a polypeptide comprising the amino acid sequence of amino acids 29-170 of SEQ ID NO:2, wherein the crystal has a space group symmetry of R32 and comprises a unit cell having the dimensions of a=b and are about 105 Å, and c is about 478 Å.

94. The crystal of claim 90, further comprising a ligand, wherein the ligand is complexed with the OX40L, wherein the OX40L comprises a polypeptide comprising the amino acid sequence of amino acids 51-183 of SEQ ID NO:3, wherein SEQ ID NO: 3 has the following mutations: N90D and N114D, and wherein the ligand comprises a polypeptide comprising the amino acid sequence of amino acids 29-170 of SEQ ID NO:2, and wherein the crystal has a space group symmetry of R32 and a unit cell having the dimensions of a=b and are about 112 Å, and c is about 233 Å.

95. A crystal comprising a hOX40 receptor, wherein the hOX40 receptor comprises a polypeptide having at least 90% sequence identity to a polypeptide comprising the amino acid sequence of amino acids 29-170 of SEQ ID NO:2, and wherein the polypeptide has at least one of the following characteristics: binds to a hOX40L or a hOX40L receptor binding fragment thereof.

96. A computer-implemented method for causing a display of a graphical three-dimensional representation of the structure of at least a portion of a crystal comprising an OX40L or structural homolog thereof, wherein the method comprises:
causing said display of said graphical three-dimensional representation by a computer system programmed with instructions for transforming structure coordinates into said graphical three-dimensional representation of said structure and for displaying said graphical three-dimensional representation,
wherein said graphical three-dimensional representation is generated by transforming said structure coordinates into said graphical three-dimensional representation of said structure,
wherein said structure coordinates comprise structure coordinates of the backbone atoms of the at least a portion of the crystal, and
wherein the OX40L is a mOX40L or a hOX40L.

97. A computer-implemented method for causing a display of a graphical three-dimensional representation of the structure of at least a portion of a crystal comprising a hOX40 receptor or structural homolog thereof, wherein the method comprises:
causing said display of said graphical three-dimensional representation by a computer system programmed with instructions for transforming structure coordinates into said graphical three-dimensional representation of said structure and for displaying said graphical three-dimensional representation,
wherein said graphical three-dimensional representation is generated by transforming said structure coordinates into said graphical three-dimensional representation of said structure,
wherein said structure coordinates comprise structure coordinates of the backbone atoms of the at least a portion of the crystal.

98. A machine-readable data storage medium comprising a data storage material encoded with machine-readable instructions for:
(a) transforming data into a graphical three-dimensional representation of the structure of at least a portion of a crystal comprising an OX40L or structural homolog thereof, wherein the OX40L is a mOX40L or a hOX40L; and
(b) causing the display of said graphical three-dimensional representation.

99. A machine-readable data storage medium comprising a data storage material encoded with machine-readable instructions for:
(a) transforming data into a graphical three-dimensional representation of the structure of at least a portion of a crystal comprising a hOX40 receptor or structural homolog thereof; and
(b) causing the display of said graphical three-dimensional representation.

100. A computer system for displaying a three-dimensional graphical representation of the structure of at least a portion of a crystal comprising an OX40L or structural homolog thereof, comprising:
(a) a machine-readable data storage medium comprising a data storage material encoded with machine-readable data, wherein said data comprise structure coordinates of the backbone atoms of the amino acids defining at least a portion of the crystal, wherein the OX40L is a mOX40L or a hOX40L;
(b) a working memory;
(c) a central processing unit coupled to said working memory and to said machine-readable data storage medium for processing said machine-readable data into said three-dimensional graphical representation; and
(d) a display coupled to said central processing unit for displaying said three-dimensional graphical representation.

101. A computer system for displaying a three-dimensional graphical representation of the structure of at least a portion of a crystal comprising a hOX40 receptor or structural homolog thereof, comprising:
(a) a machine-readable data storage medium comprising a data storage material encoded with machine-readable data, wherein said data comprise structure coordinates of the backbone atoms of the amino acids defining at least a portion of the crystal;
(b) a working memory;
(c) a central processing unit coupled to said working memory and to said machine-readable data storage medium for processing said machine-readable data into said three-dimensional graphical representation; and
(d) a display coupled to said central processing unit for displaying said three-dimensional graphical representation.

102. A polypeptide comprising the amino acid sequence starting at any one of amino acids 51 to amino acid residue 65 and ending at any one of amino acids 180 to 183 of SEQ ID NO:3, not including the polypeptide having the amino acid sequence of SEQ ID NO:1 or SEQ ID NO:3, and wherein the polypeptide binds a hOX40 receptor or a hOX40 receptor ligand binding fragment thereof.
